SEQUENCE LISTING (16 sequences)

SEQ ID NO: 1 - DNA, 24 bp, Artificial Sequence
  Synthetic Polynucleotide
atcaacgggactttccaaaatgtc

SEQ ID NO: 2 - DNA, 40 bp, Artificial Sequence
  Synthetic Polynucleotide
tttttttctagactataaccatgtaagaaagctttcttta

SEQ ID NO: 3 - DNA, 20 bp, Artificial Sequence
  Synthetic Polynucleotide
gagttagccctgcgtagcca

SEQ ID NO: 4 - DNA, 20 bp, Artificial Sequence
  Synthetic Polynucleotide
ctgccgaagcagttcttgct

SEQ ID NO: 5 - DNA, 34 bp, Artificial Sequence
  Synthetic Polynucleotide
ttttttgtcgaccagaattgatctaccatggact

SEQ ID NO: 6 - DNA, 37 bp, Artificial Sequence
  Synthetic Polynucleotide
tttttttctagactagctttctttatctagcttggtg

SEQ ID NO: 7 - DNA, 42 bp, Artificial Sequence
  Synthetic Polynucleotide
tttttttctagactataaccatgtaagaaagctttctgggct

SEQ ID NO: 8 - DNA, 27 bp, Artificial Sequence
  Synthetic Polynucleotide
tccccaatgagtgaatatatgatcaac

SEQ ID NO: 9 - DNA, 24 bp, Artificial Sequence
  Synthetic Polynucleotide
gcggtttattcggtatacaaatcg

SEQ ID NO: 10 - DNA, 59 bp, Artificial Sequence
  Synthetic Polynucleotide
ggggacaagtttgtacaaaaaagcaggcttcgccaccatggagcaaaagctcatctcag

SEQ ID NO: 11 - DNA, 55 bp, Artificial Sequence
  Synthetic Polynucleotide
ggggaccactttgtacaagaaagctgggtcagtcctttacaagcctgtaaatatg

SEQ ID NO: 12 - DNA, 20 bp, Artificial Sequence
  Synthetic Polynucleotide
taataggccagtactgatgc

SEQ ID NO: 13 - DNA, 20 bp, Artificial Sequence
  Synthetic Polynucleotide
gaatgagctcgaacatgctg

SEQ ID NO: 14 - DNA, 200 bp, Artificial Sequence
  Synthetic Polynucleotide
gctgagggggcagtccagtaggctctgggcaaacaggtcagcagagagcaagctcccgggttgggtcaccggctccccatcctctggttggaacacatcatcctccagctcctccacacactgagatggctcagcgtaatctggtacgtcgtatgggtacatctctcctgtgagggggcaacgcaggcatctgggctgct

SEQ ID NO: 15 - DNA, 20 bp, Artificial Sequence
  Synthetic Polynucleotide
agaagggaaggggagtcctt

SEQ ID NO: 16 - DNA, 25 bp, Artificial Sequence
  Synthetic Polynucleotide
cgtaatctggtacgtcgtatgggta

(19) United States
(12) Patent Application Publication
(10) Pub. No.: US 2023/0192607 A1
(43) Pub. Date: Jun. 22, 2023

(54) TRANSCRIPTIONAL ENHANCED ASSOCIATE DOMAIN (TEAD) TRANSCRIPTION FACTOR INHIBITORS AND USES THEREOF

(71) Applicant: Dana-Farber Cancer Institute, Inc., Boston, MA (US)

(72) Inventors: Nathanael S. Gray, Stanford, CA (US); Tinghu Zhang, Brookline, MA (US); Jianwei Che, Sharon, MA (US); Nicholas Paul Kwiatkowski, Brookline, MA (US); Mengyang Fan, Allston, MA (US); Wenchao LU, Brookline, MA (US)

(73) Assignee: Dana-Farber Cancer Institute, Inc., Boston, MA (US)

(21) Appl. No.: 17/789,023

(22) PCT Filed: Dec. 23, 2020
(86) PCT No.: PCT/US2020/066811

§ 371 Date: Jun. 24, 2022

Related U.S. Application Data
(60) Provisional application No. 62/953,381, filed on Dec. 24, 2019.

(51) Int. Cl. C07D 207/14; C07C 311/46; C07C 237/42; C07C 237/44; C07C 237/16; A61P 35/00

(52) U.S. Cl. CPC C07D 207/14; A61P 35/00; C07C 237/16; C07C 237/42; C07C 237/44; C07C 311/46

(57) ABSTRACT

Provided herein are compounds of (I-A), (I-B), or (II), and pharmaceutically acceptable salts, solvates, hydrates, poly-morphs, co-crystals, tautomers, stereoisomers, isotopically labeled derivatives, and prodrugs thereof. Also provided are methods, uses, and kits involving the inventive compounds and pharmaceutical compositions thereof for treating and/or preventing diseases (e.g., proliferative diseases (e.g., cancers), inflammatory diseases (e.g., fibrosis), autoimmune diseases (e.g., sclerosis)) in a subject. Provided are methods of inhibiting the activity of a transcription factor (e.g., TEAD, such as TEAD1, TEAD2, TEAD3, TEAD4) and/or inhibiting the transcription of a gene (e.g., a gene controlled or regulated by a transcription factor (e.g., TEAD)) in a subject.

### RELATED APPLICATIONS

This application claims priority under 35 U.S.C. § 119(e) to U.S. Provisional Application, U.S.S.N. 62/953,381, filed Dec. 24, 2019, which is incorporated herein by reference.

### BACKGROUND OF THE INVENTION

The Hippo signaling pathway plays key roles in organ size control and tumor suppression. YAP and transcriptional enhanced associate domain (TEAD) are major effectors of the Hippo signaling pathway. Signal transduction involves a core kinase cascade, leading to YAP (Yes1-associated protein)/TAZ (transcriptional co-activator with PDZ-binding motif) phosphorylation. Physiological or pathological inactivation leads to dephosphorylation and nuclear accumulation. Nuclear YAP/TAZ binds to TEADs to mediate target gene expression. The TEAD-YAP complex regulates organ development and amplification of oncogenic factors in many cancers (e.g., sarcoma, lung cancer, thyroid cancer, skin cancer, ovarian cancer, colorectal cancer, prostate cancer, pancreatic cancer, esophageal cancer, liver cancer, breast cancer). Several genes in the Hippo signaling pathway have been identified as tumor suppressors, and mutations in these genes have been associated with different human cancers. Additionally, elevated YAP levels have been associated with certain human cancers.

The attachment of the fatty acid palmitate to cysteine residues regulates protein trafficking, membrane localization, and signaling activities. TEAD transcription factors have been found to possess intrinsic palmitoylating enzyme-like activity and undergo autopalmitoylation. The TEAD transcription factors serve as canonical partners for the Hippo pathway effector YAP, which has been associated with resistance to targeted therapy in several contexts, including resistance to epidermal growth factor receptor (EGFR) tyrosine kinase inhibitors (TKI’s) in EGFR-mutant NSCLC (Chaib et al., 2017; Hsu et al., 2016). EGFR tyrosine kinase inhibitors (TKI’s) are the standard of care for patients with advanced EGFR mutant non-small cell lung cancer (NSCLC) (Mok et al., 2009; Rosell et al., 2012; Soria et al., 2018). However, within months to a few years, acquired resistance mechanisms inevitably develop, limiting the clinical efficacy of EGFR inhibitor treatment (Cortot and Jänne, 2014). In most cases, drug resistance to EGFR-targeted therapy arises after a dramatic initial clinical response followed by an extended time in a non-proliferative minimal residual disease (MRD), or dormant, state, with subsequent gradual emergence and growth of a drug resistant tumor. Prior preclinical studies suggest that following EGFR TKI treatment, EGFR mutant tumor cells can enter a drug tolerant state, reminiscent of dormancy in patients, allowing cells to evade apoptosis and survive under drug treatment (Hata et al., 2016; Sharma et al., 2010). Over time, the drug tolerant cells can acquire drug resistance through either mutational or non-mutational mechanisms (Hata et al., 2016). While it has been proposed that the establishment of this state is largely stochastic and dictated mostly by epigenetic mechanisms (Guler et al., 2017; Sharma et al., 2010), the mechanistic bases of how cancer cells evade the initial apoptosis in response to drug treatment - the absolute requirement to enter the drug tolerant state - or maintain tolerance in the presence of drug treatment are poorly understood.

Previous work demonstrated that despite sustained EGFR inhibition following EGFR TKI treatment of EGFR-mutant cells, reactivation of ERK ½ occurs within just a few days (Ercan et al., 2012; Tricker et al., 2015). Concomitant inhibition of MEK effectively prevents reactivation of ERK ½, results in a greater initial apoptotic response, and leads to a more durable tumor control in vitro and in vivo than single agent EGFR inhibition (Ercan et al., 2012; Tricker et al., 2015). The EGFR (osimertinib) and MEK (selumetinib) inhibitor combination has been studied in patients resistant to prior EGFR TKI’s and is also under evaluation as an initial therapy for advanced EGFR-mutant NSCLC in a phase II clinical trial (NCT03392246; Ramalingam et al., 2019). However, even with this combination, acquired resistance still develops either through bypass of EGFR/MEK inhibition, or by unknown mechanisms that do not involve reactivation of EGFR downstream signaling (Tricker et al., 2015). Due to the key regulatory functions of transcription factors TEAD, YAP, and the TEAD-YAP complex in development, cell growth and proliferation, tissue homeostasis, and regeneration, it is important to develop modulators of the activity of these transcription factors (e.g., TEAD, YAP), including selective modulators (e.g., selective inhibitors), for use as research tools as well as therapeutic agents in the treatment of various diseases associated with these transcription factors. It is also important to develop therapeutic agents for treating proliferative diseases associated with these transcription factors (e.g., TEAD, YAP) that may be resistant to certain anti-proliferative agents (e.g., cancers resistant to inhibitors of EGFR and/or MEK) via combination therapy using modulators of the transcription factors TEAD, YAP, EGFR, and/or MEK.

### SUMMARY OF THE INVENTION

This disclosure is based in part on the discovery that eradicating tumor dormancy that develops following oncogene-targeted therapy, including after epidermal growth factor receptor (EGFR) tyrosine kinase inhibitor (TKI) treatment of a cancer, for example, lung cancer (e.g., EGFR-mutant non-small cell lung cancer (NSCLC)), is an attractive therapeutic strategy. However, the mechanisms governing the establishment of tumor dormancy are poorly understood. It was recently observed that the blockade of ERK½ reactivation following EGFR TKI treatment by combined EGFR/MEK inhibition uncovers cells that survive by entering a senescence-like dormant state, characterized by extensive epigenetic remodeling and high YAP/TEAD activity. YAP/TEAD trigger an epithelial-to-mesenchymal transition (EMT) program and engage the EMT transcription factor SLUG to directly repress pro-apoptotic BMF, limiting drug-induced apoptosis. Pharmacological co-inhibition of YAP or TEAD, or genetic deletion of YAP1, all deplete dormant cells by enhancing EGFR/MEK inhibitor-induced apoptosis. Thus, YAP activation can promote the survival of EGFR-mutant NSCLC cells in the chronic absence of EGFR signaling. Eradicating this surviving cell population, for example, by inhibiting TEAD and/or YAP, enhances the efficacy of targeted therapies which could ultimately lead to prolonged treatment responses in cancer patients.

Described herein are TEAD inhibitor compounds of Formulae (I-A), (I-B), and (II), and pharmaceutically acceptable salts, co-crystals, tautomers, stereoisomers, solvates, hydrates, polymorphs, isotopically enriched derivatives, and prodrugs thereof, and mixtures thereof.

The compounds of Formulae (I-A), (I-B), and (II), and pharmaceutically acceptable salts, co-crystals, tautomers, stereoisomers, solvates, hydrates, polymorphs, isotopically enriched derivatives, prodrugs, and compositions thereof, may inhibit the activity of a transcription factor (e.g., TEAD) in a biological sample or subject. In certain embodiments, the transcription factor is a transcriptional enhanced associate domain (TEAD). In certain embodiments, the compound of Formula (I-A), (I-B), or (II) is selective for a specific TEAD (e.g., TEAD1, TEAD2, TEAD3, TEAD4) compared to other TEADs. Described herein are methods of using the inventive compounds, and pharmaceutically acceptable salts, co-crystals, tautomers, stereoisomers, solvates, hydrates, polymorphs, isotopically enriched derivatives, prodrugs, and compositions thereof, to study the inhibition of a transcription factor (e.g., TEAD, such as TEAD1, TEAD2, TEAD3, TEAD4).,The inventive compounds described herein may also be used as therapeutics for the prevention and/or treatment of diseases associated with the overexpression and/or aberrant (e.g., increased or unwanted) activity of a transcription factor (e.g., TEAD, such as TEAD1, TEAD2, TEAD3, TEAD4). The compounds described herein may be useful in treating and/or preventing a disease or condition, e.g., in treating and/or preventing a disease (e.g., proliferative disease (e.g., cancer, benign neoplasm), inflammatory disease (e.g., fibrosis), autoimmune disease (e.g., sclerosis)), in a subject in need thereof. For treating and/or preventing a disease described herein (e.g., proliferative disease (e.g., cancer, benign neoplasm, for example, a cancer resistant to a modulator of another transcription factor (e.g., YAP, EGFR, MEK)), inflammatory disease (e.g., fibrosis), autoimmune disease (e.g., sclerosis)), the compounds described herein may be optionally administered in combination with an additional pharmaceutical agent, for example, modulators of other transcription factors (e.g., YAP, EGFR, MEK), for inhibiting a transcription factor (e.g., TEAD, such as TEAD1, TEAD2, TEAD3, TEAD4) in a subject and/or biological sample, and for inhibiting the transcription of a gene (e.g., a gene controlled or regulated by a transcription factor (e.g., TEAD, such as TEAD1, TEAD2, TEAD3, TEAD4)) in a subject and/or biological sample. Also provided are uses, pharmaceutical compositions, and kits including a compound described herein.

In one aspect, the present disclosure provides compounds of Formula (I-A):

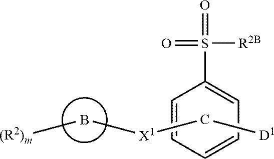

and pharmaceutically acceptable salts, co-crystals, tautomers, stereoisomers, solvates, hydrates, polymorphs, isotopically enriched derivatives, and prodrugs thereof, wherein R², R^{2B}, X¹, Ring B, m, and D¹ are as defined herein. D¹ is a warhead which in some embodiments binds a TEAD (e.g., TEAD1, TEAD2, TEAD3, TEAD4). In certain embodiments, the warhead non-covalently binds to a TEAD, e.g., TEAD1, TEAD2, TEAD3, TEAD4. In certain embodiments, the warhead covalently binds to a TEAD, e.g., TEAD1, TEAD2, TEAD3, TEAD4.

Exemplary compounds of Formula (I-A), include, but are not limited to:

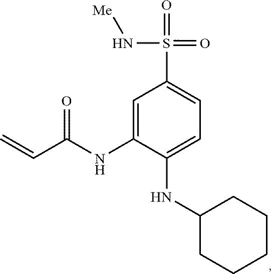

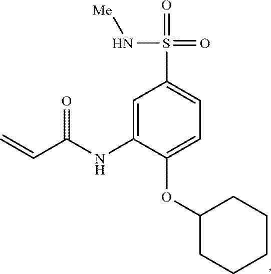

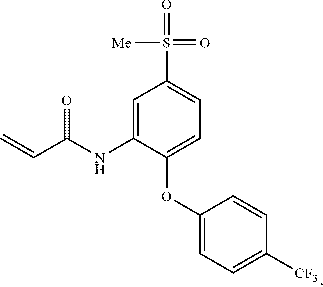

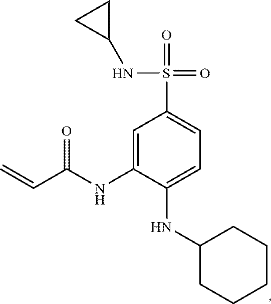

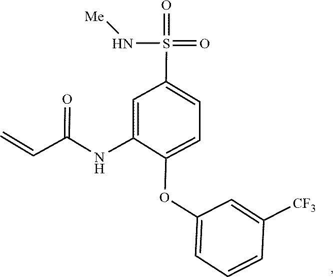

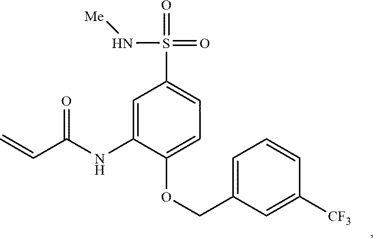

and pharmaceutically acceptable salts, co-crystals, tautomers, stereoisomers, solvates, hydrates, polymorphs, isotopically enriched derivatives, and prodrugs thereof.

Exemplary compounds of Formula (I-A), include, but are not limited to:

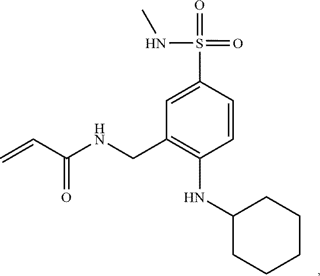

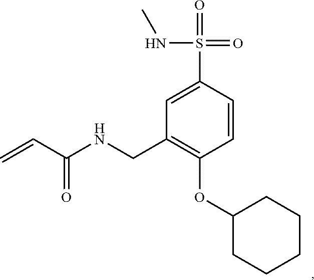

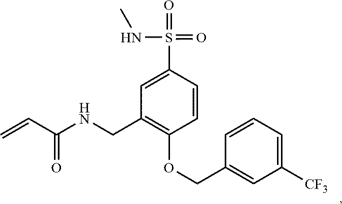

and pharmaceutically acceptable salts, co-crystals, tautomers, stereoisomers, solvates, hydrates, polymorphs, isotopically enriched derivatives, and prodrugs thereof.

In one aspect, the present disclosure provides compounds of Formula (I-B):

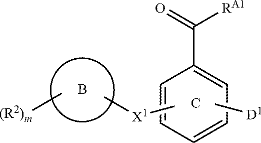

and pharmaceutically acceptable salts, co-crystals, tautomers, stereoisomers, solvates, hydrates, polymorphs, isotopically enriched derivatives, and prodrugs thereof, wherein R², R^{A1}, X¹, Ring B, m, and D¹ are as defined herein. D¹ is a warhead which in some embodiments binds a TEAD (e.g., TEAD1, TEAD2, TEAD3, TEAD4). In certain embodiments, the warhead non-covalently binds to a TEAD, e.g., TEAD1, TEAD2, TEAD3, TEAD4. In certain embodiments, the warhead covalently binds to a TEAD, e.g., TEAD1, TEAD2, TEAD3, TEAD4. In certain embodiments, the warhead non-covalently binds to a TEAD, e.g., TEAD1, TEAD2, TEAD3, TEAD4.

Exemplary compounds of Formula (I-B), include, but are not limited to:

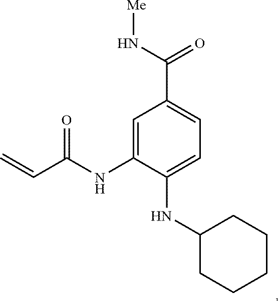

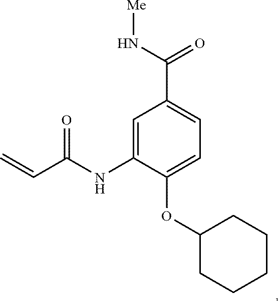

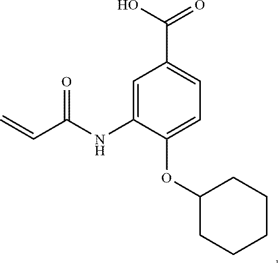

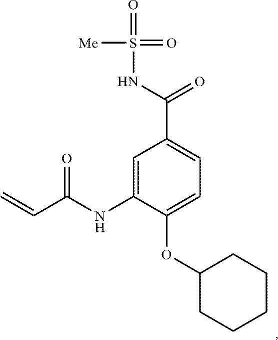

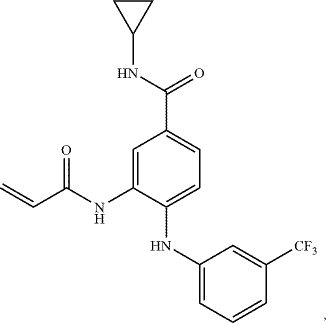

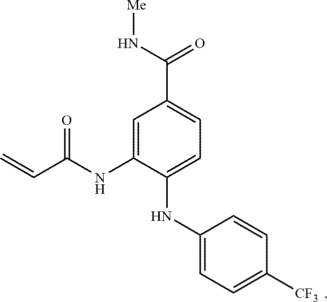

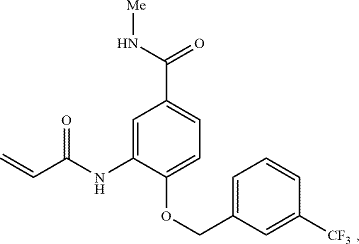

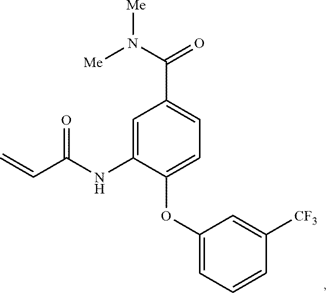

and pharmaceutically acceptable salts, co-crystals, tautomers, stereoisomers, solvates, hydrates, polymorphs, isotopically enriched derivatives, and prodrugs thereof.

Exemplary compounds of Formula (I-B), include, but are not limited to:

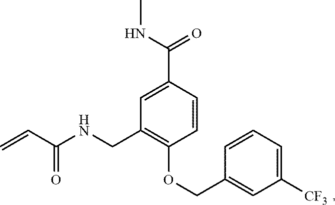

and pharmaceutically acceptable salts, co-crystals, tautomers, stereoisomers, solvates, hydrates, polymorphs, isotopically enriched derivatives, and prodrugs thereof.

In certain embodiments, the compound of Formula (I-B) is not of formula:

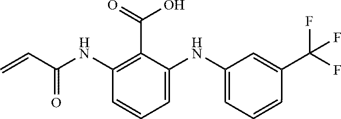 [SMILES: C=CC(=O)Nc1cccc(Nc2cccc(C(F)(F)F)c2)c1C(=O)O]

or

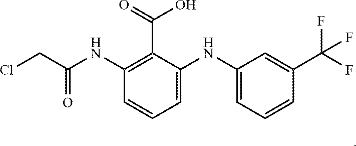

In one aspect, the present disclosure provides compounds of Formula (II):

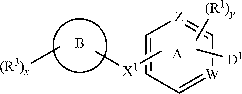

and pharmaceutically acceptable salts, co-crystals, tautomers, stereoisomers, solvates, hydrates, polymorphs, isotopically enriched derivatives, and prodrugs thereof, wherein R¹, R², X¹, Ring B, W, Z, x, y, and D¹ are as defined herein. D¹ is a warhead which in some embodiments binds a TEAD (e.g., TEAD1, TEAD2, TEAD3, TEAD4). In certain embodiments, the warhead non-covalently binds to a TEAD, e.g., TEAD1, TEAD2, TEAD3, TEAD4. In certain embodiments, the warhead covalently binds to a TEAD, e.g., TEAD1, TEAD2, TEAD3, TEAD4.

Exemplary compounds of Formula (II), include, but are not limited to:

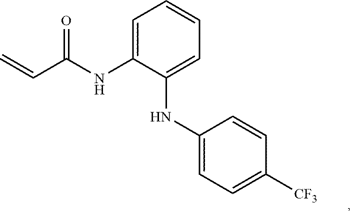

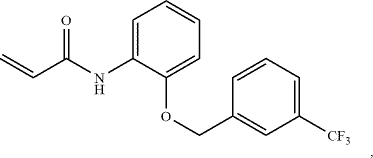

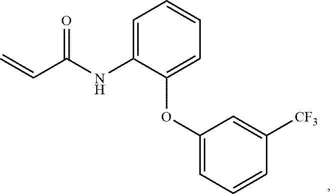

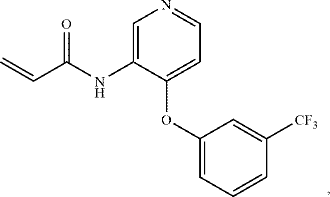

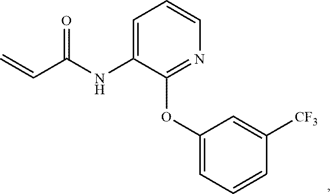

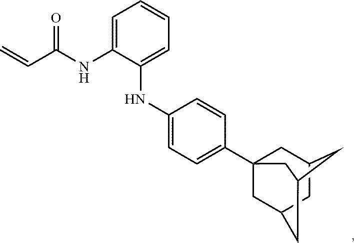

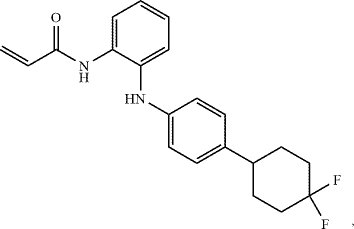

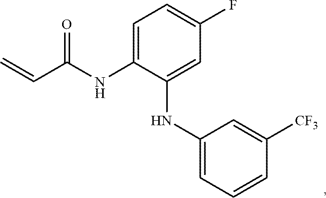

and pharmaceutically acceptable salts, co-crystals, tautomers, stereoisomers, solvates, hydrates, polymorphs, isotopically enriched derivatives, and prodrugs thereof.

In another aspect, the present disclosure provides pharmaceutical compositions including a compound described herein, and optionally a pharmaceutically acceptable excipient. In certain embodiments, the pharmaceutical compositions described herein include a therapeutically or prophylactically effective amount of a compound described herein. The pharmaceutical composition may be useful for treating and/or preventing a disease (e.g., a proliferative disease, inflammatory disease, autoimmune disease) in a subject in need thereof, inhibiting the activity of a transcription factor (e.g., TEAD, such as TEAD1, TEAD2, TEAD3, TEAD4), or inhibiting the transcription of a gene (e.g., a gene controlled or regulated by a transcription factor (e.g., TEAD, such as TEAD1, TEAD2, TEAD3, TEAD4) in a subject, and/or biological sample (e.g., tissue, cell). In certain embodiments, the proliferative disease is cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma). In certain embodiments, the cancer is a sarcoma (e.g., Kaposi’s sarcoma). In certain embodiments, the cancer is a carcinoma. In certain embodiments, the cancer is lung cancer (e.g., non-small cell lung cancer, mesothelioma). In certain embodiments, the cancer has a mutation in a gene of the Hippo signaling pathway. In certain embodiments, the cancer has a mutation in EGFR. In certain embodiments, the cancer has a mutation in MEK. In certain embodiments, the cancer is an EGFR-mutant non-small cell lung cancer. In certain embodiments, the cancer is resistant to certain anti-proliferative agents (e.g., cancers resistant to inhibitors of EGFR and/or MEK). In certain embodiments, the cancer is resistant to inhibitors of EGFR (e.g., osimertinib) and/or inhibitors of MEK (e.g., trametinib). In certain embodiments, the cancer is resistant to tyrosine kinase inhibitors (TKI’s). In certain embodiments, the disease is an inflammatory disease (e.g., fibrosis). In certain embodiments, the disease is an autoimmune disease (e.g., sclerosis).

In another aspect, described herein are methods for treating and/or preventing a disease (e.g., a proliferative disease, inflammatory disease, autoimmune disease) using a compound described herein, which may be optionally administered in combination with an additional pharmaceutical agent, for example, modulators of other transcription factors (e.g., YAP, EGFR, MEK). Exemplary proliferative diseases which may be treated include diseases associated with the overexpression or the increased activity of a TEAD, e.g., a proliferative disease, such as cancer, or a cancer resistant to a modulator (e.g., inhibitor) of another transcription factor (e.g., YAP, EGFR, MEK). In certain embodiments, the cancer is selected from the group consisting of sarcoma, lung cancer, thyroid cancer, breast cancer, liver cancer, pancreatic cancer, gastric cancer, ovarian cancer, colon cancer, colorectal cancer, skin cancer, esophageal cancer; carcinoma. In certain embodiments, the cancer is lung cancer (e.g., non-small cell lung cancer, mesothelioma). In certain embodiments, the cancer is a sarcoma (e.g., Kaposi’s sarcoma). In certain embodiments, the disease is an inflammatory disease (e.g., fibrosis). In certain embodiments, the disease is an autoimmune disease (e.g., sclerosis).

Another aspect relates to methods of inhibiting the activity of a transcription factor (e.g., TEAD, such as TEAD1, TEAD2, TEAD3, TEAD4) using a compound described herein in a biological sample (e.g., cell, tissue), which may be optionally administered in combination with an additional pharmaceutical agent, for example, modulators of other transcription factors (e.g., YAP, EGFR, MEK). In another aspect, described herein are methods of inhibiting the activity of a transcription factor (e.g., TEAD, such as TEAD1, TEAD2, TEAD3, TEAD4) using a compound described herein in a subject. In certain embodiments, the method involves the inhibition of TEAD (e.g., TEAD2). Another aspect relates to methods of inhibiting the transcription of a gene (e.g., a gene controlled or regulated by a transcription factor (e.g., TEAD, such as TEAD1, TEAD2, TEAD3, TEAD4))) using a compound described herein, which may be optionally administered in combination with an additional pharmaceutical agent, for example, modulators of other transcription factors (e.g., YAP, EGFR, MEK).

Described herein are methods for administering to a subject in need thereof an effective amount of a compound, or pharmaceutical composition thereof, as described herein, which may be optionally administered in combination with an additional pharmaceutical agent, for example, modulators of other transcription factors (e.g., YAP, EGFR, MEK). Also described are methods for contacting a biological sample (e.g., tissue, cell) with an effective amount of a compound, or pharmaceutical composition thereof, as described herein, which may be optionally administered in combination with an additional pharmaceutical agent, for example, modulators of other transcription factors (e.g., YAP, EGFR, MEK). In certain embodiments, a method described herein further includes administering to the subject an additional pharmaceutical agent. In certain embodiments, a method described herein further includes contacting the biological sample (e.g., tissue, cell) with an additional pharmaceutical agent (e.g., an anti-proliferative agent). In certain embodiments, the additional pharmaceutical agent is a modulator of another transcription factor (e.g., YAP, EGFR, MEK). In certain embodiments, the additional pharmaceutical agent is a transcription inhibitor (e.g., an inhibitor of EGFR and/or MEK). In certain embodiments, the additional pharmaceutical agent is a kinase inhibitor. In certain embodiments, the additional pharmaceutical agent is an agent for treating lung cancer (e.g., non-small cell lung cancer (NSCLC)).

In yet another aspect, the present disclosure provides compounds of Formulae (I-A), (I-B), and (II), and pharmaceutically acceptable salts, solvates, hydrates, polymorphs, co-crystals, tautomers, stereoisomers, isotopically labeled derivatives, prodrugs, and compositions thereof, which may be optionally administered in combination with an additional pharmaceutical agent, for example, modulators of other transcription factors (e.g., YAP, EGFR, MEK), for use in the treatment of a disease (e.g., a proliferative disease, inflammatory disease, autoimmune disease) in a subject. In yet another aspect, the present disclosure provides compounds of Formulae (I-A), (I-B), and (II), and pharmaceutically acceptable salts, solvates, hydrates, polymorphs, co-crystals, tautomers, stereoisomers, isotopically labeled derivatives, prodrugs, and compositions thereof, which may be optionally administered in combination with an additional pharmaceutical agent, for example, modulators of other transcription factors (e.g., YAP, EGFR, MEK), for use in inhibiting the activity of a transcription factor (e.g., TEAD, such as TEAD1, TEAD2, TEAD3, TEAD4)), or inhibiting the transcription of a gene (e.g., a gene controlled or regulated by a transcription factor (e.g., TEAD, such as TEAD1, TEAD2, TEAD3, TEAD4))) in a subject and/or biological sample (e.g., tissue, cell).

Another aspect of the present disclosure relates to kits comprising a container with a compound, or pharmaceutical composition thereof, as described herein. The kits described herein may include a single dose or multiple doses of the compound or pharmaceutical composition. The kits may be useful in a method of the disclosure. In certain embodiments, the kit further includes instructions for using the compound or pharmaceutical composition. A kit described herein may also include information (e.g. prescribing information) as required by a regulatory agency, such as the U.S. Food and Drug Administration (FDA).

The details of one or more embodiments of the invention are set forth herein. Other features, objects, and advantages of the invention will be apparent from the Detailed Description, Examples, Figures, and Claims.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, aredescribed in Thomas Sorrell, Organic Chemistry, University Science Books, Sausalito, 1999;Smith and March, March’s Advanced Organic Chemistry, 5^{th} Edition, John Wiley & Sons,Inc., New York, 2001; Larock, Comprehensive Organic Transformations, VCH Publishers,Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The invention additionally encompasses compounds described herein as individual isomers substantially free of other isomers, and alternatively, as mixtures of various isomers.

When a range of values is listed, it is intended to encompass each value and sub-range within the range. For example, “C₁₋₆” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

A “hydrocarbon chain” refers to a substituted or unsubstituted divalent alkyl, alkenyl, or alkynyl group. A hydrocarbon chain includes at least one chain, each node (“carbon unit”) of which including at least one carbon atom, between the two radicals of the hydrocarbon chain; (2) optionally one or more hydrogen atoms on the chain(s) of carbon atoms; and (3) optionally one or more substituents (“non-chain substituents,” which are not hydrogen) on the chain(s) of carbon atoms. A chain of carbon atoms consists of consecutively connected carbon atoms (“chain atoms”) and does not include hydrogen atoms or heteroatoms. However, a non-chain substituent of a hydrocarbon chain may include any atoms, including hydrogen atoms, carbon atoms, and heteroatoms. For example, hydrocarbon chain —C^{A}H(C^{B}H₂C^{C}H₃)— includes only one carbon unit C^{A}. For example, hydrocarbon chain —C^{A}H(C^{B}H₂C^{C}H₃)— includes only one carbon unit C^{A}, one hydrogen atom on C^{A}, and non-chain substituent —(C^{B}H₂C^{C}H₃). The term “Cₓ hydrocarbon chain,” wherein x is a positive integer, refers to a hydrocarbon chain that includes x number of carbon unit(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

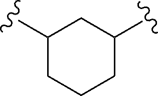

is a C₃ hydrocarbon chain. When a range of values is used, e.g., a C₁₋₆ hydrocarbon chain, the meaning of the range is as described herein. For example, a C₃₋₁₀ hydrocarbon chain refers to a hydrocarbon chain where the number of chain atoms of the shortest chain of carbon atoms immediately between the two radicals of the hydrocarbon chain is 3, 4, 5, 6, 7, 8, 9, or 10. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of a unsubstituted and unsaturated hydrocarbon chain. In certain embodiments, the hydrocarbon chain is unsubstituted (e.g., —C≡C— or —(CH₂)₄—). In certain embodiments, the hydrocarbon chain is substituted (e.g., —CH(C₂H₅)— and —CF₂—). Any two substituents on the hydrocarbon chain may be joined to form an optionally substituted carbocyclyl, optionally substituted heterocyclyl, optionally substituted aryl, or optionally substituted heteroaryl ring. For instance,

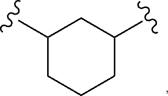

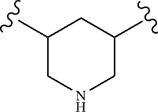

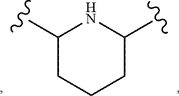

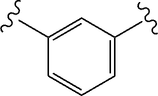

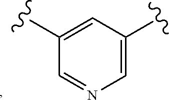

, and

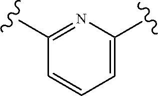

are all examples of a hydrocarbon chain. In contrast, in certain embodiments

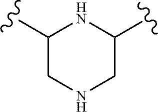

and

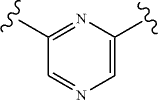

are not within the scope of the hydrocarbon chains described herein. When a chain atom of a Cₓ hydrocarbon chain is replaced with a heteroatom, the resulting group is referred to as a Cₓ hydrocarbon chain wherein a chain atom is replaced with a heteroatom, as opposed to a Cₓ₋₁ hydrocarbon chain. For example,

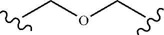

is a C₃ hydrocarbon chain wherein one chain atom is replaced with an oxygen atom.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Other examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₅) and the like. Further examples of alkyl groups include n-heptyl (C₇), n-octyl (C₅), n-dodecyl (C₁₂), and the like. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₀ alkyl. In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2-20 carbon atoms (“C₂₋₂₀ alkenyl”), 2 to 12 carbon atoms (“C₂₋₁₂ alkenyl”), 2-10 carbon atoms (“C₂₋₁₀ alkenyl”), 2-8 carbon atoms (“C₂₋₈ alkenyl”), 2-7 carbon atoms (“C₂₋₇ alkenyl”), or 2-6 carbon atoms (“C₂₋₆ alkenyl”). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms(“C₂₋₄ alkenyl”). In some embodiments, an alkenyl group has 2 to 3 carbon atoms(“C₂₋₃ alkenyl”). In some embodiments, an alkenyl group has 2 to 4 carbon atoms(“C₂₋₄ alkenyl”). In some embodiments, an alkenyl group has 1 to 3 carbon atoms (“C₂₋₃ alkenyl”). In some embodiments, an alkenyl group has 1 to 2 carbon atoms (“C₁₋₂ alkenyl”). In some embodiments, an alkenyl group has 1 carbon atoms (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

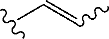

) may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). In some embodiments, an alkynyl group has 1-2 carbon atoms (“C₁₋₂ alkynyl”). In some embodiments, an alkynyl group has 1 carbon atom (“C₁ alkynyl”). In some embodiments, an alkynyl group has 2-20 carbon atoms (“C₂₋₁₂ alkynyl”), 2 to 12 carbon atoms (“C₂₋₁₂ alkynyl”), 2-10 carbon atoms (“C₂₋₁₀ alkynyl”), 2-8 carbon atoms (“C₂₋₈ alkynyl”), 2-7 carbon atoms (“C₂₋₇ alkynyl”), or 2-6 carbon atoms (“C₂₋₆ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 10 ring carbon atoms (“C₅-In some embodiments, a carbocyclyl group has 5 to 6 ring carbon atoms (“C₅₋₆ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”) and can be saturated or can be partially unsaturated. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl group is an unsubstituted C₃₋₁₄ carbocyclyl. In certain embodiments, the carbocyclyl group is a substituted C₃₋₁₄ carbocyclyl. In certain embodiments, the carbocyclyl includes 0, 1, or 2C═C double bonds in the carbocyclic ring system, as valency permits.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. In certain embodiments, the carbocyclyl group is an unsubstituted C₃₋₁₄ carbocyclyl. In certain embodiments, the carbocyclyl group is a substituted C₃₋₁₄ carbocyclyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from the group consisting of nitrogen, oxygen, sulfur, boron, phosphorus, and silicon (“3-10 membered heterocyclyl”). Heterocyclyl″ or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from the group consisting of nitrogen, oxygen, sulfur, boron, phosphorus, and silicon (“5-10 membered heterocyclyl”). In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms , wherein each heteroatom is independently selected from the group consisting of nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from the group consisting of nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing 2 heteroatoms include dioxolanyl, oxathiolanyl and dithiolanyl. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from the group consisting of nitrogen, oxygen, and sulfur (“5-10 membered heteroaryl”). The term “heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from the group consisting of nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from the group consisting of nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from the group consisting of nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of alkyl and heteroaryl and refers to an optionally substituted alkyl group substituted by an optionally substituted heteroaryl group.

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined herein. Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups are further referred to using the suffix -ene, e.g., alkylene, alkenylene, alkynylene, carbocyclylene, heterocyclylene, arylene, and heteroarylene.

A group is optionally substituted unless expressly provided otherwise. The term “optionally substituted” refers to substituted or unsubstituted. Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})2, —N(R^{bb})2, —N(R^{bb})₃⁺X^{—}, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})2, —P(═O)(OR^{cc})2, —OP(═O)(R^{aa})₂, —OP(═O)(OR^{cc})2, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X^{—}, —P(OR^{cc})₃⁺X^{—}, —P(R^{cc})₄, —P(OR^{cc})₄, —OP(R^{cc})₂, —OP(R^{cc})₃⁺X^{—}, —OP(OR^{cc})₂, —OP(OR^{cc})₃⁺X^{—}, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X^{—}, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂,—NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C_{6-1O} aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
   each instance of R^{gg} is, independently, halogen, —CN, —NO2, —N₃, —SO₂H, —SO₃H, —OH, —OC_{1—6} alkyl, —ON(C_{1—6} alkyl)₂, —N(C_{1—6} alkyl)₂, —N(C_{1—6} alkyl)₃⁺X^{—}, —NH(C_{1—6} alkyl)₂⁺X^{—}, —NH₂(C_{1—6} alkyl) ⁺X^{—}, -NH₃⁺X^{—}, —N(OC_{1—6} alkyl)(C_{1—6} alkyl), —N(OH)(C_{1—6} alkyl), —NH(OH), —SH, —SC_{1—6} alkyl, —SS(C_{1—6} alkyl), —C(═O)(C_{1—6} alkyl), —CO₂H, —CO₂(C_{1—6} alkyl), —OC(═O)(C_{1—6} alkyl), —OCO₂(C_{1—6} alkyl), —C(═O)NH₂, —C(═O)N(C_{1—6} alkyl)₂, —OC(═O)NH(C_{1—6} alkyl), —NHC(═O)( C_{1—6} alkyl), —N(C_{1—6} alkyl)C(═O)( C_{1—6} alkyl), —NHCO₂(C_{1—6} alkyl), —NHC(═O)N(C_{1—6} alkyl)₂, —NHC(═O)NH(C_{1—6} alkyl), —NHC(═O)NH₂, —C(═NH)O(C_{1—6} alkyl), —OC(═NH)(C_{1—6} alkyl), —OC(═NH)OC_{1—6} alkyl, —C(═NH)N(C_{1—6} alkyl)₂, —C(═NH)NH(C_{1—6} alkyl), —C(═NH)NH₂, —OC(═NH)N(C_{1—6} alkyl)₂, —OC(NH)NH(C_{1—6} alkyl), —OC(NH)NH₂, —NHC(NH)N(C_{1—6} alkyl)₂, —NHC(═NH)NH₂, —NHSO₂(C_{1—6} alkyl), —SO₂N(C_{1—6} alkyl)₂, —SO₂NH(C_{1—6} alkyl), —SO₂NH₂, —SO₂C₁_{—6} alkyl, —SO₂OC_{1—6} alkyl, —OSO₂C₁_{—6} alkyl, —SOC_{1—6} alkyl, —Si(C_{1—6} alkyl)₃, —OSi(C_{1—6} alkyl)₃—C(═S)N(C_{1—6} alkyl)₂, C(═S)NH(C_{1—6} alkyl), C(═S)NH₂, —C(═O)S(C_{1—6} alkyl), —C(═S)SC_{1—6} alkyl, —SC(═S)SC_{1—6} alkyl, —P(═O)(OC_{1—6} alkyl)₂, —P(═O)(C_{1—6} alkyl)₂, —OP(═O)(C_{1—6} alkyl)₂, —OP(═O)(OC_{1—6} alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF6⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO2, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

“Halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di- aliphaticamino, mono- or di- heteroaliphaticamino, mono- or di- alkylamino, mono- or di- heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkoxy” or “alkoxyl” refers to a radical of the formula: -O-alkyl.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, — SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide. For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-dit-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate. In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′ -phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) ₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4′′-tris(benzoyloxyphenyl)methyl, 3-(imidazol-l-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4′′-tris(benzoyloxyphenyl)methyl, 4,4′-Dimethoxy-3‴-[N-(imidazolylmethyl) ]trityl Ether (IDTr-OR), 4,4′-Dimethoxy-3‴-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻—P,(OR^{cc})₂—P(OR^{cc})₃⁺X⁻, —P(═O)(R^{aa}₂, —P(═O)(OR^{cc})₂, and —P(═O)(N(R^{bb}) ₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

As used herein, a “leaving group” (LG) is an art-understood term referring to a molecular fragment that departs with a pair of electrons in a heterolytic bond cleavage, wherein the molecular fragment is an anion or neutral molecule. A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), or trifluoromethanesulfonate (triflate, —OTf). In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, amines, ammonia, alcohols, ether moieties, sulfur-containing moieties, thioether moieties, zinc halides, magnesium moieties, diazonium salts, and copper moieties. Other non-limiting examples of leaving groups are water, ammonia, alcohols, ether moieties, thioether moieties, zinc halides, magnesium moieties, diazonium salts, and copper moieties.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I-A), (I-B), or (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (-)-isomers respectively). A chiral compound can exist as either individual enantiomer or as a mixture thereof. A mixture containing equal proportions of the enantiomers is called a “racemic mixture.”

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “polymorphs” refers to a crystalline form of a compound (or a salt, hydrate, or solvate thereof) in a particular crystal packing arrangement. The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds, including derivatives of the compounds of Formulae (I-A), (I-B), and (II), which have cleavable groups and become by solvolysis or under physiological conditions the compounds of Formulae (I-A), (I-B), and (II) which are pharmaceutically active in vivo. Such examples include, but are not limited to, ester derivatives and the like. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see Bundgard, H., Design of Prodrugs, pp. 7-9, 21-24, Elsevier, Amsterdam 1985). Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. A non-human animal may be a transgenic animal.

The terms “administer,” “administering,” or “administration” refer to implanting, absorbing, ingesting, injecting, inhaling, or otherwise introducing an inventive compound or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The term “prevent,” “preventing,” or “prevention” refers to a prophylactic treatment of a subject who does not have and did not have a disease but is at risk of developing the disease or is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

The term “inhibition,” “inhibiting,” “inhibit,” or “inhibitor” refer to the ability of a compound to reduce, slow, halt, or prevent activity of a particular biological process (e.g., a transcription factor) in a cell relative to vehicle.

An “effective amount” of a compound of Formula (I-A), (I-B), or (II) refers to an amount sufficient to elicit the desired biological response, i.e., treating the condition, for example, inhibiting TEAD. As will be appreciated by those of ordinary skill in this art, the effective amount of a compound of Formula (I-A), (I-B), or (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I-A), (I-B), or (II) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding a transcription factor (e.g., TEAD, such as TEAD1, TEAD2, TEAD3, TEAD4) and/or inhibiting the transcription factor (e.g., TEAD, such as TEAD1, TEAD2, TEAD3, TEAD4). In certain embodiments, a prophylactically effective amount is an amount sufficient for binding a transcription factor (e.g., TEAD, such as TEAD1, TEAD2, TEAD3, TEAD4) and/or inhibiting the transcription factor (e.g., TEAD, such as TEAD1, TEAD2, TEAD3, TEAD4). In certain embodiments, a prophylactically effective amount is an amount sufficient for preventing a disease and/or condition (e.g., proliferative disease, inflammatory disease, autoimmune disease). In certain embodiments, a prophylactically effective amount is an amount sufficient for binding a transcription factor (e.g., TEAD, such as TEAD1, TEAD2, TEAD3, TEAD4), and treating and/or preventing a disease and/or condition (e.g., proliferative disease, inflammatory disease, autoimmune disease). In certain embodiments, a prophylactically effective amount is an amount sufficient for binding a transcription factor (e.g., TEAD, such as TEAD1, TEAD2, TEAD3, TEAD4) and/or inhibiting the transcription factor (e.g., TEAD, such as TEAD1, TEAD2, TEAD3, TEAD4).

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes, such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, lymphoma, non-Hodgkin’s lymphoma, Waldenstrom macroglobulinemia, MYD88-mutated Waldenstrom macroglobulinemia, activated B-cell diffuse large B-cell lymphoma, leukemia, sarcoma, lung cancer, thyroid cancer, breast cancer, liver cancer, pancreatic cancer, gastric cancer, ovarian cancer, colon cancer, colorectal cancer, skin cancer, esophageal cancer, and carcinoma. Exemplary proliferative diseases include cancers (i.e., “malignant neoplasms,” , sarcoma, lung cancer, thyroid cancer, breast cancer, liver cancer, pancreatic cancer, gastric cancer, ovarian cancer, colon cancer, colorectal cancer, skin cancer, esophageal cancer; carcinoma), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor’s neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary original tumor to another organ or tissue and is typically identifiable by the presence of a “secondary tumor” or “secondary cell mass” of the tissue type of the primary original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman’s Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi’s sarcoma, multiple idiopathic hemorrhagic sarcoma); endometrial cancer (e.g., uterine cancer, uterine sarcoma); esophageal cancer (e.g., adenocarcinoma of the esophagus, Barrett’s adenocarcinoma); Ewing’s sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemialsmall lymphocytic lymphoma (CLL/SLL), MYD88-mutated Waldenstrom macroglobulinemia, activated B-cell (ABC) diffuse large B-cell lymphoma, mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström’s macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphomalleukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms’ tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g.,bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget’s disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget’s disease of the vulva).

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren’s syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto’s thyroiditis, Graves’ disease, Goodpasture’s disease, mixed connective tissue disease, sclerosing cholangitis, inflammatory bowel disease, Crohn’s disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener’s granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation. In certain embodiments, the inflammatory disorder is fibrosis, and the fibrosis is idiopathic pulmonary fibrosis, liver cirrhosis, cystic fibrosis, systemic sclerosis, progressive kidney disease, or cardiovascular fibrosis.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture’s disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture’s syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener’s granulomatosis, microscopic polyangiitis), uveitis, Sjogren’s syndrome, Crohn’s disease, Reiter’s syndrome, ankylosing spondylitis, Lyme disease, Guillain-Barré syndrome, Hashimoto’s thyroiditis, and cardiomyopathy. In certain embodiments, the autoimmune disorder is sclerosis. In certain embodiments, the sclerosis is systemic sclerosis (scleroderma) or multiple sclerosis.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

A “transcription factor” is a type of protein that is involved in the process of transcribing DNA into RNA, and/or modulating the transcription of one or more genes. Transcription factors can work independently or with other proteins in a complex to either stimulate or repress transcription. Transcription factors contain at least one DNA-binding domain that give them the ability to bind to specific sequences of DNA. Other proteins such as coactivators, chromatin remodelers, histone acetyltransferases, histone deacetylases, kinases, and methylases are also essential to gene regulation, but lack DNA-binding domains, and therefore are not transcription factors. These exemplary human transcription factors include, but are not limited to, YAP, EGFR, MEK, TEAD (e.g., TEAD1, TEAD2, TEAD3, TEAD4), AC008770.3, AC023509.3, AC092835.1, AC138696.1, ADNP, ADNP2, AEBP1, AEBP2, AHCTF1, AHDC1, AHR, AHRR, AIRE, AKAP8, AKAP8L, AKNA, ALX1, ALX3, ALX4, ANHX, ANKZF1, AR, ARGFX, ARHGAP35, ARID2, ARID3A, ARID3B, ARID3C, ARID5A, ARID5B, ARNT, ARNT2, ARNTL, ARNTL2, ARX, ASCL1, ASCL2, ASCL3, ASCL4, ASCL5, ASH1L, ATF1, ATF2, ATF3, ATF4, ATF5, ATF6, ATF6B, ATF7, ATMIN, ATOH1, ATOH7, ATOH8, BACH1, BACH2, BARHL1, BARHL2, BARX1, BARX2, BATF, BATF2, BATF3, BAZ2A, BAZ2B, BBX, BCL11A, BCL11B, BCL6, BCL6B, BHLHA15, BHLHA9, BHLHE22, BHLHE23, BHLHE40, BHLHE41, BNC1, BNC2, BORCS-MEF2B, BPTF, BRF2, BSX, C11orf95, CAMTA1, CAMTA2, CARF, CASZ1, CBX2, CC2D1A, CCDC169-SOHLH2, CCDC17, CDC5L, CDX1, CDX2, CDX4, CEBPA, CEBPB, CEBPD, CEBPE, CEBPG, CEBPZ, CENPA, CENPB, CENPBD1, CENPS, CENPT, CENPX, CGGBP1, CHAMP1, CHCHD3, CIC, CLOCK, CPEB1, CPXCR1, CREB1, CREB3, CREB3L1, CREB3L2, CREB3L3, CREB3L4, CREB5, CREBL2, CREBZF, CREM, CRX, CSRNP1, CSRNP2, CSRNP3, CTCF, CTCFL, CUX1, CUX2, CXXC1, CXXC4, CXXC5, DACH1, DACH2, DBP, DBX1, DBX2, DDIT3, DEAF1, DLX1, DLX2, DLX3, DLX4, DLX5, DLX6, DMBX1, DMRT1, DMRT2, DMRT3, DMRTA1, DMRTA2, DMRTB1, DMRTC2, DMTF1, DNMT1, DNTTIP1, DOT1L, DPF1, DPF3, DPRX, DR1, DRAP1, DRGX, DUX1, DUX3, DUX4, DUXA, DZIP1, E2F1, E2F2, E2F3, E2F4, E2F5, E2F6, E2F7, E2F8, E4F1, EBF1, EBF2, EBF3, EBF4, EEA1, EGR1, EGR2, EGR3, EGR4, EHF, ELF1, ELF2, ELF3, ELF4, ELF5, ELK1, ELK3, ELK4, EMX1, EMX2, EN1, EN2, EOMES, EPAS1, ERF, ERG, ESR1, ESR2, ESRRA, ESRRB, ESRRG, ESX1, ETS1, ETS2, ETV1, ETV2, ETV3, ETV3L, ETV4, ETV5, ETV6, ETV7, EVX1, EVX2, FAM170A, FAM200B, FBXL19, FERD3L, FEV, FEZF1, FEZF2, FIGLA, FIZ1, FLI1, FLYWCH1, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXM1, FOXN1, FOXN2, FOXN3, FOXN4, FOXO1, FOX03, FOXO4, FOXO6, FOXP1, FOXP2, FOXP3, FOXP4, FOXQ1, FOXR1, FOXR2, FOXS1, GABPA, GATA1, GATA2, GATA3, GATA4, GATA5, GATA6, GATAD2A, GATAD2B, GBX1, GBX2, GCM1, GCM2, GFI1, GFI1B, GLI1, GLI2, GLI3, GLI4, GLIS1, GLIS2, GLIS3, GLMP, GLYR1, GMEB1, GMEB2, GPBP1, GPBP1L1, GRHL1, GRHL2, GRHL3, GSC, GSC2, GSX1, GSX2, GTF2B, GTF2I, GTF2IRD1, GTF2IRD2, GTF2IRD2B, GTF3A, GZF1, HAND1, HAND2, HBP1, HDX, HELT, HES1, HES2, HES3, HES4, HES5, HES6, HES7, HESX1, HEY1, HEY2, HEYL, HHEX, HIC1, HIC2, HIF1A, HIF3A, HINFP, HIVEP1, HIVEP2, HIVEP3, HKR1, HLF, HLX, HMBOX1, HMG20A, HMG20B, HMGA1, HMGA2, HMGN3, HMX1, HMX2, HMX3, HNF1A, HNF1B, HNF4A, HNF4G, HOMEZ, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HSF1, HSF2, HSF4, HSF5, HSFX1, HSFX2, HSFY1, HSFY2, IKZF1, IKZF2, IKZF3, IKZF4, IKZF5, INSM1, INSM2, IRF1, IRF2, IRF3, IRF4, IRF5, IRF6, IRF7, IRF8, IRF9, IRX1, IRX2, IRX3, IRX4, IRX5, IRX6, ISL1, ISL2, ISX, JAZF1, JDP2, JRK, JRKL, JUN, JUNB, JUND, KAT7, KCMF1, KCNIP3, KDM2A, KDM2B, KDM5B, KIN, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KMT2B, L3MBTL1, L3MBTL3, L3MBTL4, LBX1, LBX2, LCOR, LCORL, LEF1, LEUTX, LHX1, LHX2, LHX3, LHX4, LHX5, LHX6, LHX8, LHX9, LIN28A, LIN28B, LIN54, LMX1A, LMX1B, LTF, LYL1, MAF, MAFA, MAFB, MAFF, MAFG, MAFK, MAX, MAZ, MBD1, MBD2, MBD3, MBD4, MBD6, MBNL2, MECOM, MECP2, MEF2A, MEF2B, MEF2C, MEF2D, MEIS1, MEIS2, MEIS3, MEOX1, MEOX2, MESP1, MESP2, MGA, MITF, MIXL1, MKX, MLX, MLXIP, MLXIPL, MNT, MNX1, MSANTD1, MSANTD3, MSANTD4, MSC, MSGN1, MSX1, MSX2, MTERF1, MTERF2, MTERF3, MTERF4, MTF1, MTF2, MXD1, MXD3, MXD4, MXI1, MYB, MYBL1, MYBL2, MYC, MYCL, MYCN, MYF5, MYF6, MYNN, MYOD1, MYOG, MYPOP, MYRF, MYRFL, MYSM1, MYT1, MYT1L, MZF1, NACC2, NAIF1, NANOG, NANOGNB, NANOGP8, NCOA1, NCOA2, NCOA3, NEUROD1, NEUROD2, NEUROD4, NEUROD6, NEUROG1, NEUROG2, NEUROG3, NFAT5, NFATC1, NFATC2, NFATC3, NFATC4, NFE2, NFE2L1, NFE2L2, NFE2L3, NFE4, NFIA, NFIB, NFIC, NFIL3, NFIX, NFKB1, NFKB2, NFX1, NFXL1, NFYA, NFYB, NFYC, NHLH1, NHLH2, NKRF, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NME2, NOBOX, NOTO, NPAS1, NPAS2, NPAS3, NPAS4, NR0B1, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRF1, NRL, OLIG1, OLIG2, OLIG3, ONECUT1, ONECUT2, ONECUT3, OSR1, OSR2, OTP, OTX1, OTX2, OVOL1, OVOL2, OVOL3, PA2G4, PATZ1, PAX1, PAX2, PAX3, PAX4, PAX5, PAX6, PAX7, PAX8, PAX9, PBX1, PBX2, PBX3, PBX4, PCGF2, PCGF6, PDX1, PEG3, PGR, PHF1, PHF19, PHF20, PHF21A, PHOX2A, PHOX2B, PIN1, PITX1, PITX2, PITX3, PKNOX1, PKNOX2, PLAG1, PLAGL1, PLAGL2, PLSCR1, POGK, POU1F1, POU2AF1, POU2F1, POU2F2, POU2F3, POU3F1, POU3F2, POU3F3, POU3F4, POU4F1, POU4F2, POU4F3, POU5Fl, POU5F1B, POU5F2, POU6F1, POU6F2, PPARA, PPARD, PPARG, PRDM1, PRDM10, PRDM12, PRDM13, PRDM14, PRDM15, PRDM16, PRDM2, PRDM4, PRDM5, PRDM6, PRDM8, PRDM9, PREB, PRMT3, PROP1, PROX1, PROX2, PRR12, PRRX1, PRRX2, PTF1A, PURA, PURB, PURG, RAG1, RARA, RARB, RARG, RAX, RAX2, RBAK, RBCK1, RBPJ, RBPJL, RBSN, REL, RELA, RELB, REPIN1, REST, REXO4, RFX1, RFX2, RFX3, RFX4, RFX5, RFX6, RFX7, RFX8, RHOXF1, RHOXF2, RHOXF2B, RLF, RORA, RORB, RORC, RREB1, RUNX1, RUNX2, RUNX3, RXRA, RXRB, RXRG, SAFB, SAFB2, SALL1, SALL2, SALL3, SALL4, SATB1, SATB2, SCMH1, SCML4, SCRT1, SCRT2, SCX, SEBOX, SETBP1, SETDB1, SETDB2, SGSM2, SHOX, SHOX2, SIM1, SIM2, SIX1, SIX2, SIX3, SIX4, SIX5, SIX6, SKI, SKIL, SKOR1, SKOR2, SLC2A4RG, SMAD1, SMAD3, SMAD4, SMAD5, SMAD9, SMYD3, SNAI1, SNAI2, SNAI3, SNAPC2, SNAPC4, SNAPC5, SOHLH1, SOHLH2, SON, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPDEF, SPEN, SPI1, SPIB, SPIC, SPZ1, SRCAP, SREBF1, SREBF2, SRF, SRY, ST18, STAT1, STAT2, STAT3, STAT4, STAT5A, STA5B, STT6, T, TAL1, TAL2, TBP, TBPL1, TBPL2, TBR1, TBX1, TBX10, TBX15, TBX18, TBX19, TBX2, TBX20, TBX21, TBX22, TBX3, TBX4, TBX5, TBX6, TCF12, TCF15, TCF20, TCF21, TCF23, TCF24, TCF3, TCF4, TCF7, TCF7L1, TCF7L2, TCFL5, TEAD1, TEAD2, TEAD3, TEAD4, TEF, TERB1, TERF1, TERF2, TET1, TET2, TET3, TFAP2A, TFAP2B, TFAP2C, TFAP2D, TFAP2E, TFAP4, TFCP2, TFCP2L1, TFDP1, TFDP2, TFDP3, TFE3, TFEB, TFEC, TGIF1, TGIF2, TGIF2LX, TGIF2LY, THAP1, THAP10, THAP11, THAP12, THAP2, THAP3, THAP4, THAP5, THAP6, THAP7, THAP8, THAP9, THRA, THRB, THYN1, TIGD1, TIGD2, TIGD3, TIGD4, TIGD5, TIGD6, TIGD7, TLX1, TLX2, TLX3, TMF1, TOPORS, TP53, TP63, TP73, TPRX1, TRAFD1, TRERF1, TRPS1, TSC22D1, TSHZ1, TSHZ2, TSHZ3, TTF1, TWIST1, TWIST, UBP1, UNCX, USF1, USF2, USF3, VAX1, VAX2, VDR, VENTX, VEZF1, VSX1, VSX2, WIZ, WT1, XBP1, XPA, YBX1, YBX2, YBX3, YY1, YY2, ZBED1, ZBED2, ZBED3, ZBED4, ZBED5, ZBED6, ZBED9, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB9, ZC3H8, ZEB1, ZEB2, ZFAT, ZFHX2, ZFHX3, ZFHX4, ZFP1, ZFP14, ZFP2, ZFP28, ZFP3, ZFP30, ZFP37, ZFP41, ZFP42, ZFP57, ZFP62, ZFP64, ZFP69, ZFP69B, ZFP82, ZFP90, ZFP91, ZFP92, ZFPM1, ZFPM2, ZFX, ZFY, ZGLP1, ZGPAT, ZHX1, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT4, ZNF10, ZNF100, ZNF101, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, NF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF721, ZNF724, ZNF726, ZNF727, ZNF728, ZNF729, ZNF730, ZNF732, ZNF735, ZNF736, ZNF737, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF821, ZNF823, ZNF827, ZNF829, ZNF83, ZNF830, ZNF831, ZNF835, ZNF836, ZNF837, ZNF84, ZNF841, ZNF843, ZNF844, ZNF845, ZNF846, ZNF85, ZNF850, ZNF852, ZNF853, ZNF860, ZNF865, ZNF878, ZNF879, ZNF880, ZNF883, ZNF888, ZNF891, ZNF90, ZNF91, ZNF92, ZNF93, ZNF98, ZNF99, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZUFSP, ZXDA, ZXDB, ZXDC, and ZZZ3.

The term “TEAD” refers to transcriptional enhanced associate domain (TEAD) transcription factors. TEADs are primary transcription factors for the Yes-associated protein (YAP)/PDZ-binding domain (TAZ) transcription coactivators of the Hippo signaling pathway. Examples of TEADs include, but are not limited to, TEAD1, TEAD2, TEAD3, and TEAD4. For TEAD2, exemplary NCBI sequences from GenBank are: NM_001256660.2 (Homo sapiens) and NM_001256659.2 (Homo sapiens). Exemplary genes controlled or regulated by TEADs include, but are not limited to, TGF, CYR61, WNT5A/B, DKK1, TGFB2, BMP4, AREG, EGFR, PD-L1, MYC, LATS2, amino acid transporters SLC38A1/SLC7A5, and glucose transporter GLUT3. TEADs bind to DNA sequences including, but not limited to, MCAT DNA sequences, and the 5′-GGAATG-3′ consensus sequence.

### BRIEF DESCRIPTION OF THE DRAWINGS

FIGS. 1A-1K show combined EGFR/MEK inhibition promotes a senescence-like dormant state. FIG. 1A shows confluency percentage over time, showing the proliferation of PC-9 cells treated with DMSO, 100 nM osimertinib (O) alone or in combination with 30 nM trametinib (T), where the combined treatment with osimertinib and trametinib is “OT.” Upon treatment under the conditions discussed above for FIG. 1A, FIG. 1B shows images of control cells (at 1 week) or dormant PC-9 cells (at 15 weeks). Scale bar, 200 µm. FIG. 1C shows cells were treated as in FIG. 1A for 6 weeks followed by drug washout. FIG. 1D shows western blot analysis of EGFR downstream signaling following treatment with OT for indicated times or 21 days followed by drug washout (rebound). FIG. 1E shows percentage barcodes shared among replicates following indicated treatments in barcoded PC-9 cells. FIG. 1F shows relative abundance of individual barcodes. Shared and unique indicate barcodes shared by >2 or ≤2 replicates, respectively. In FIG. 1F, shared data is shown in darker grey; unique data is shown in lighter grey. FIG. 1G shows Gene Set Enrichment Analysis (GSEA) of Hallmark gene sets comparing dormant cells versus (vs.) DMSO-treated control cells. Normalized Enrichment Scores (NES) for gene sets with FDR<0.1 in at least two cell lines are shown. FIG. 1H shows senescence-associated β-galactosidase (SA-β-gal) staining of cells treated as indicated for 10 days. Scale bar, 100 µm. FIG. 1I shows quantification of the cells in FIG. 1H. FIG. 1J shows GSEA of senescence signature comparing dormant, OT-treated PC-9 cells vs. control cells. FIG. 1K shows immunofluorescence (IF) staining for H3K9Me³ in control cells or dormant cells treated with OT for 10 days. Scale bar, 20 µm. Mean ± SEM are shown in all plots except, as in FIG. 1I, where mean ± SD are shown. ANOVA, as in FIG. 1I, or t-test, as in FIG. 1K, were used for statistical analyses. ***, P<0.001; **, P<0.01. See also FIGS. 8A-8C, FIGS. 9A-9B, and FIGS. 10A-10D.

FIGS. 2A-2J show the establishment of cell dormancy following EGFR/MEK inhibition is critically dependent on activation of YAP/TEAD. FIG. 2A shows principal component analysis of ATAC-seq data from cells treated as indicated for two weeks. FIG. 2B shows ATAC-seq signal intensities centered on up-regulated (UP) or down-regulated (DOWN) peaks in dormant, osimertinib and trametinib (OT)-treated cells vs. DMSO-treated control cells. FIG. 2C shows analysis for enriched transcription factor motifs, FIG. 2D shows GSEA of YAP/TEAD signature (Zhang et al., 2009; see References below), and FIG. 2E on the left shows: ATAC-seq signal intensities centered on up-regulated (UP) or down-regulated (DOWN) peaks in OT-treated vs. osimertinib-treated cells. FIG. 2E on the right shows: Analysis for transcription factor motifs enriched in up-regulated peaks. FIG. 2F shows QPCR analysis of YAP target gene expression. FIG. 2G shows regrowth of EGFR-mutant NSCLC cells after washout following a three-week treatment with the indicated drug combinations. FIG. 2H shows Western blot analysis of YAP protein levels in YAP1 knock-out (KO) and control (CTRL) cells. FIG. 2I shows confluency over time, showing the proliferation of cells in FIG. 2H treated as indicated for 21 days, followed by drug washout. FIG. 2J shows the tumor volume in mice bearing CTRL or YAP1 KO cell xenograft tumors were treated with vehicle or OT followed by treatment cessation and follow-up. Data with 6/8 live mice per group are plotted. Right: tumor volumes at time of regrowth, indicated by an arrow. Mean ± SEM are shown in all plots except in FIG. 2F, where mean ± SD are shown. ANOVA was used for statistical analyses in all but FIG. 2J, where t-test was used. ***, P<0.001; *, P<0.05. See also FIGS. 11A-11E.

FIGS. 3A-3J show YAP activation is necessary for cancer cell viability upon combined EGFR/MEK inhibition. FIG. 3A shows normalized YAP activity following indicated treatments in PC-9 cells transduced with a fluorescent YAP/Hippo pathway reporter (PC-9 YAP reporter cells). FIG. 3B shows IF staining for YAP nuclear localization following the indicated treatments. FIG. 3C shows normalized YAP activity and apoptosis in PC-9 YAP reporter cells treated with osimertinib and trametinib (OT). FIG. 3D shows analysis of overlap between YAP^{high} cells (red) and apoptotic cells (green) after 80 h of treatment in PC-9 YAP reporter cells. FIG. 3E shows apoptosis in PC-9 cells treated with the indicated drugs or drug combinations. FIG. 3F shows apoptosis in EGFR-mutant NSCLC cells treated as indicated. Peak apoptosis values over 72 hours are shown. FIG. 3G shows apoptosis in YAP1 knock-out (KO) or control (CTRL) cells treated as indicated. FIG. 3H on the left shows: Western blot analysis of YAP protein levels in YAP1 KO cells transduced with wild-type YAP1. FIG. 3H on the right shows: cells treated with OT and analyzed as in FIG. 2G. Only data from drug-treated cells is shown. FIG. 3I shows proportions of YAP^{high} cells in PC-9 YAP reporter cell populations treated as indicated. FIG. 3J shows different means for EGFR-mutant NSCLC cells to avoid apoptosis following EGFR inhibition. Mean ± SEM are shown in all plots except as in FIG. 3I, where SD is shown. ANOVA was used for statistical analyses in all but FIG. 3D, where Fisher’s exact test was used. ***, P<0.001. See also FIG. 12.

FIGS. 4A-4I show YAP-high, senescence-like dormant state also occurs in vivo. FIG. 4A shows growth curves of the tumor volumes for PC-9 xenograft tumors harvested for single-cell RNA-sequencing (scRNA-seq) and immunohistochemistry (IHC). FIG. 4B shows a fluorescence-activated cell sorting (FACS) sorting scheme of live and dead cells used to obtain scRNA-seq samples from the dissociated xenograft tumors. FIG. 4C shows YAP, EMT and Fridman senescence signature enrichment in single cells from the xenograft tumors. FIGS. 4D-4E show IHC staining for YAP in the xenograft tumors, as in FIG. 4E, or in residual tumors from EGFR^{L858/T790M} mice following 2-week treatment with vehicle or osimertinib. FIG. 4F shows quantification of FIG. 4D and FIG. 4F. FIG. 4G shows quantification of infiltrating T-cells in the same tumors as in FIG. 4E based in CD4/CD8 IHC. FIGS. 4H-4I show IHC staining for YAP and pERK in WZ4002- or WZ4002/trametinib-resistant tumors from EGFR^{L858/T790M} mice, as in FIG. 4H, or in a residual tumor of an EGFR-mutant NSCLC patient following treatment with osimertinib/selumetinib for 11 months as in FIG. 4I. Kolmogorov-Smirnov Test, as in FIG. 4C, ANOVA, as in FIG. 4F when more than two groups and FIG. 4H, or t-test, as in FIG. 2F when two groups, FIG. 4G, or FIG. 4I, were used for statistical analyses. ***, P<0.001; **, P<0.01; n.s., not significant. See also FIGS. 13A-13C.

FIGS. 5A-5I show YAP mediates the evasion of apoptosis by repressing the induction of pro-apoptotic BMF. FIG. 5A shows Western blot analysis of EGFR downstream signaling in the indicated proteins (YAP, pEGFR, EGFR, pAKT, pERK, ERK, pS6, S6, BIM, tubulin) following 24 hour treatment with osimertinib, trametinib, or the combination of osimertinib and trametinib (OT) as indicated, in PC-9 cells and HCC4006. FIG. 5B shows RNA-seq samples used in FIG. 5C. FIG. 5C shows the expression of genes regulating apoptosis in OT-treated YAP1 KO cells vs. OT-treated CTRL cells. Colors indicate log2 fold change values with p<0.001. FIG. 5D shows the QPCR analysis of BMF expression in CTRL or YAP1 KO cells treated as indicated for 24 hours in vitro or 3 days in vivo. FIG. 5E shows schematic representation of the endogenous BMF locus in PC-9 HA-BMF cells. FIG. 5F shows Western blot analysis of BMF, BIM, and YAP expression in PC-9 HA-BMF cells transfected with non-targeting (NT) or YAP siRNA and treated as indicated for 24 hours. FIG. 5G shows QPCR analysis of BMF expression in CTRL or YAP1 KO cells transduced as indicated, and following treatment with either DMSO or OT for 24 hours. FIG. 5H shows peak apoptosis over 72 hour treatment in PC-9 and HCC4006 cells transfected with NT or BMF siRNA. FIG. 5I shows the mechanism of YAP/TEAD-mediated suppression of apoptosis in EGFR-mutant NSCLC cells following EGFR/MEK inhibition. Mean ± SD are shown in all plots except FIG. 5H, where mean ± SEM is shown. ANOVA was used for statistical analyses. ***, P<0.001; **, P<0.01; n.s., not significant (P>0.05). See also FIGS. 14A-14G.

FIGS. 6A-6I show YAP represses BMF induction by engaging EMT transcription factor SLUG. FIG. 6A shows GSEA of EMT signature in YAP1 knock-out (KO) vs. control cells treated with osimertinib and trametinib (OT) for 24 hours. FIG. 6B shows QPCR analysis of EMT transcription factor expression in untreated EGFR-mutant NSCLC cells. FIG. 6C sows co-immunoprecipitation analysis of the interaction between YAP, TEAD, and SLUG in PC-9 cells following treatment with DMSO or OT for 48 h. FIG. 6D shows Western blot analysis of YAP and SLUG protein levels in PC-9 or HCC4006 cells transfected with non-targeting (NT), YAP or SLUG siRNA. FIG. 6E show QPCR analysis of BMF expression in cells in FIG. 6D following 24 hour treatment with DMSO or OT. FIG. 6F shows apoptosis in cells in FIG. 6D following treatment with DMSO or OT. FIG. 6G shows number of peaks called by MACS2 (FDR<0.01). FIG. 6H shows ChIP-seq signal traces in BMF locus. H3K27Ac was used to identify enhancer regions. FIG. 6I shows the mechanism by which YAP/TEAD/SLUG complex represses BMF expression upon combined EGFR/MEK inhibition. Mean ± SD, as in FIG. 6E, or mean ± SEM, as in FIG. 6F, are shown. ANOVA was used for statistical analyses. ***, P<0.001; **, P<0.01.

FIGS. 7A-7I show the development of novel covalent TEAD inhibitors to target YAP dependency upon combined EGFR/MEK inhibition. FIG. 7A shows YAP1 mutants and viability used in the rescue experiment in FIG. 7B. FIG. 7B shows viability (Cell Titer Glo) of CTRL cells or PC-9 YAP1 KO cells transduced with YAP1 mutants, as in FIG. 7A, following 72 hour treatment with osimertinib and trametinib (OT). FIG. 7C on the top shows: the structure of compound MYF-01-37. FIG. 7C on the bottom shows: MYF-01-37 binding to the palmitoylation pocket in TEAD1 based on molecular docking. The cysteine 359 targeted by MYF-01-37 is indicated. FIG. 7D shows the effect of MYF-01-37 or the corresponding reversible control on YAP/TEAD interaction. FIG. 7E on the left shows: Western bot analysis of the expression of myc-tagged TEAD1 in PC-9 cells transduced as indicated. FIG. 7E on the right shows: QPCR analysis of CTGF expression after 24 hour treatment with compound XAV939 or MYF-01-37 in the transduced PC-9 cells. The structure of compound XAV939 is

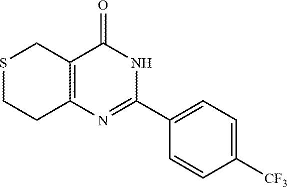 [SMILES: O=c1[nH]c(-c2ccc(C(F)(F)F)cc2)nc2c1CSCC2]

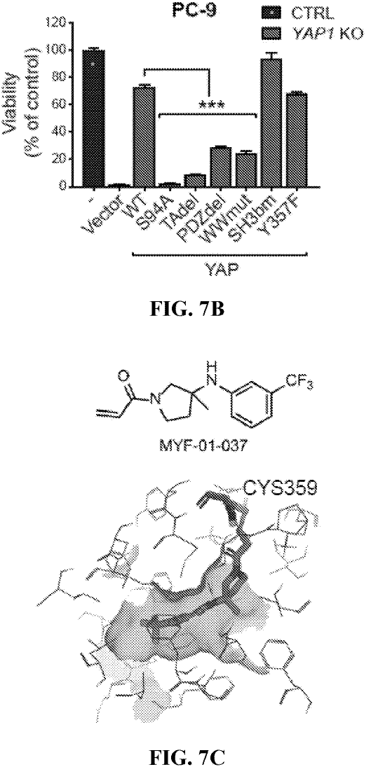

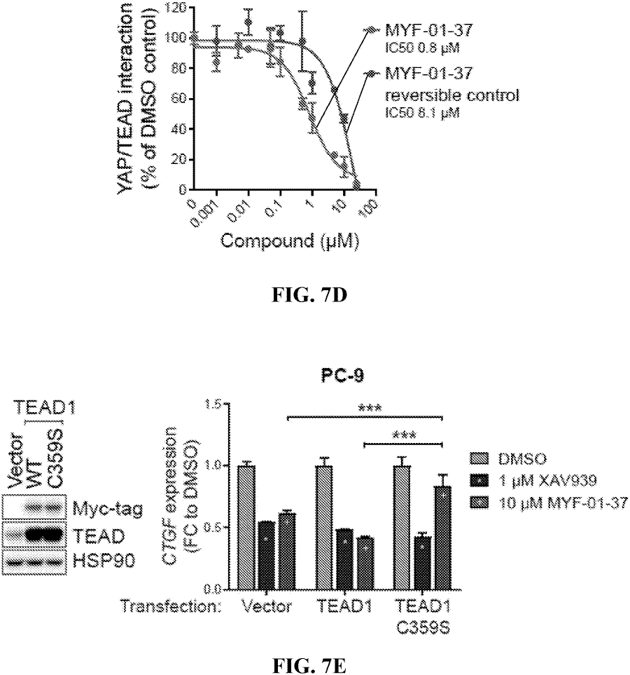

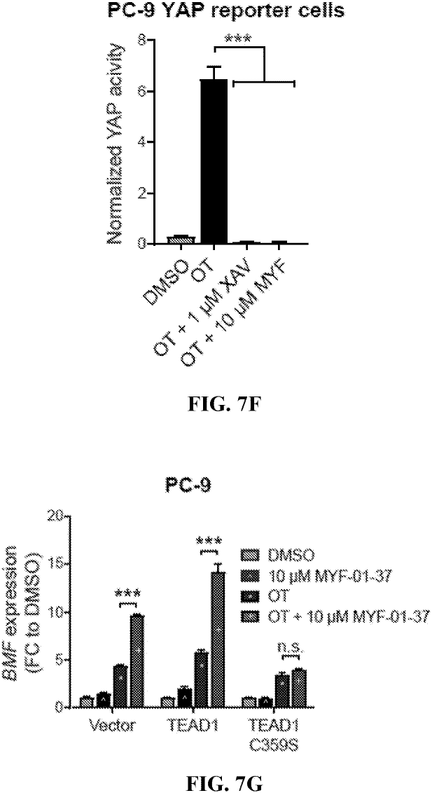

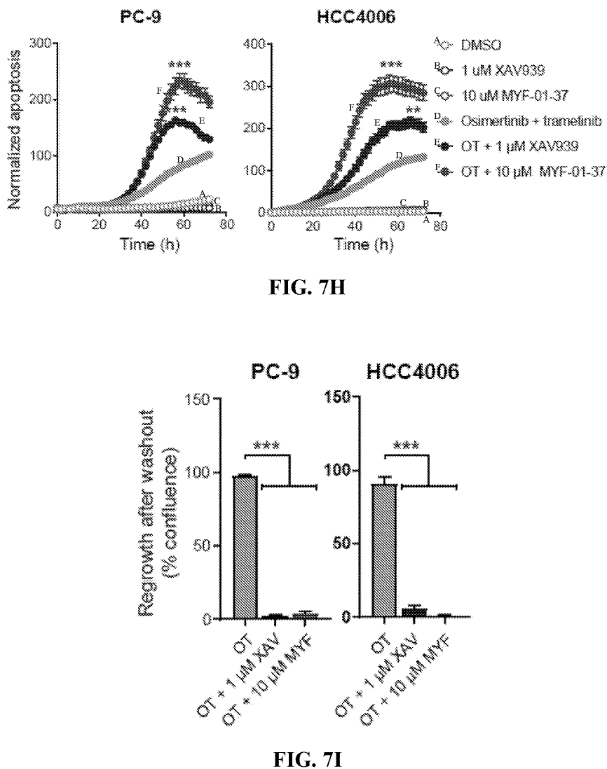

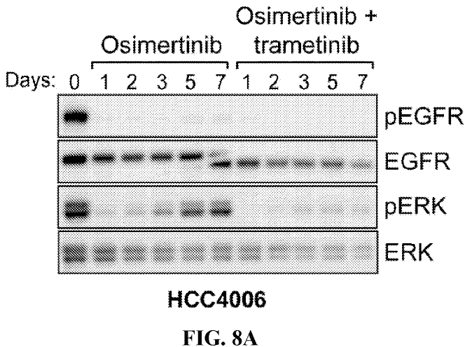

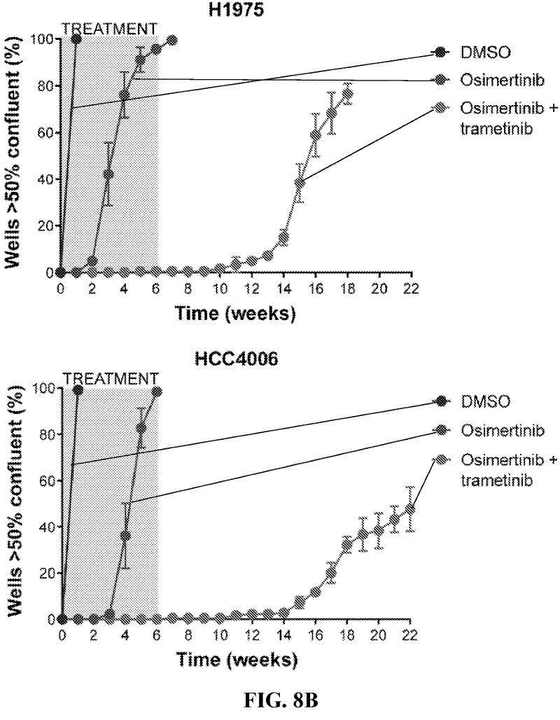

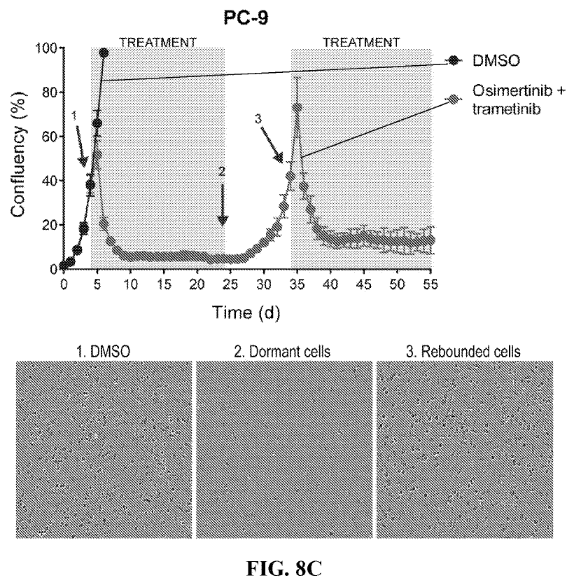

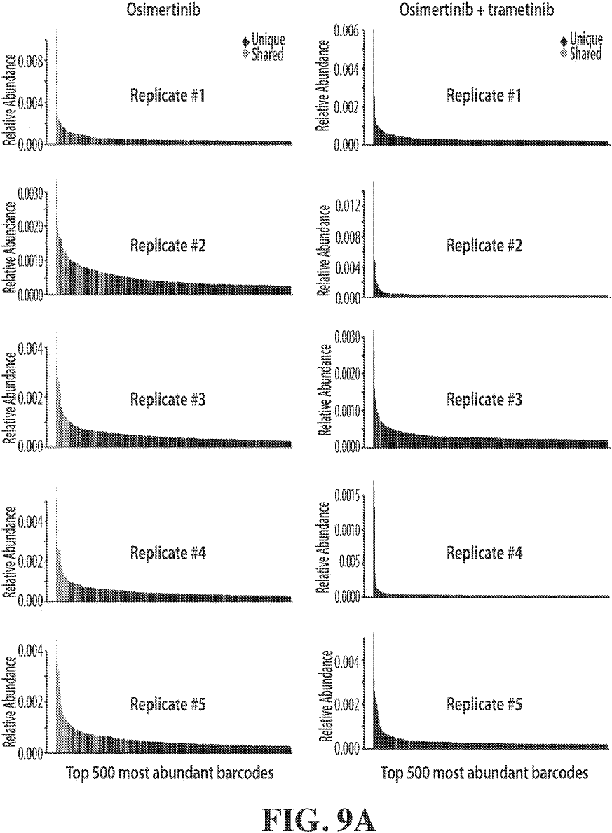

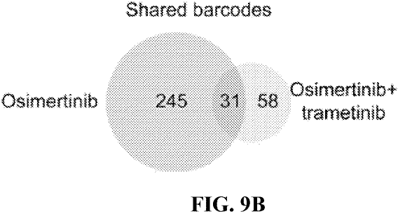

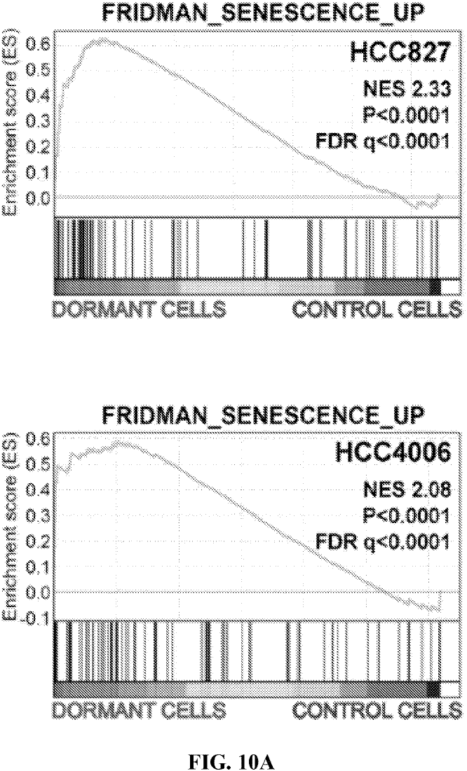

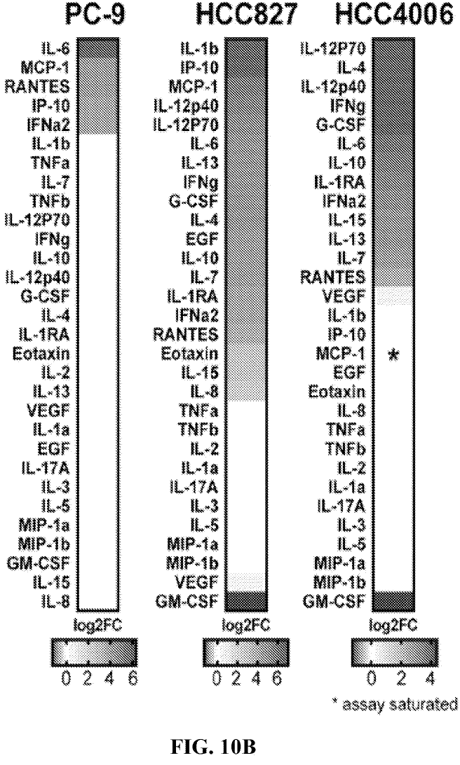

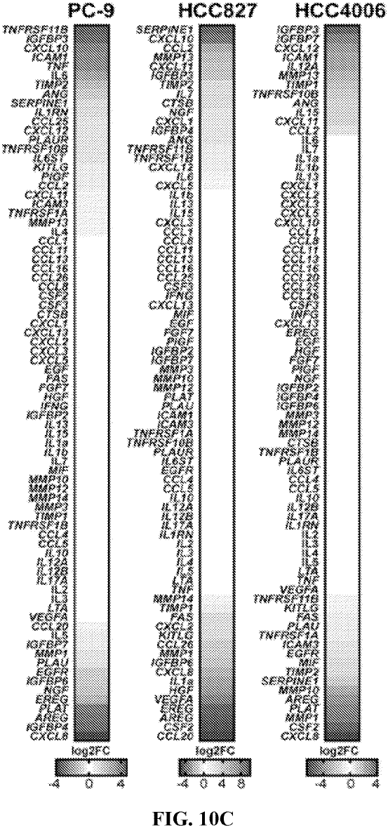

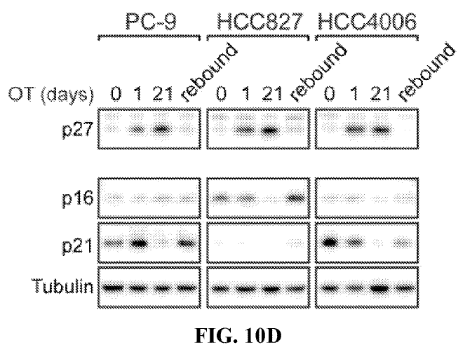

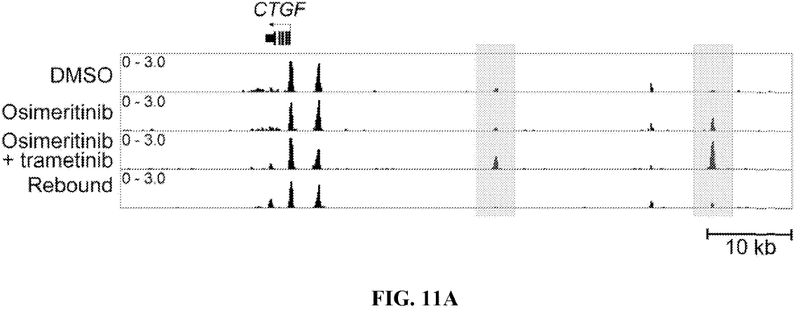

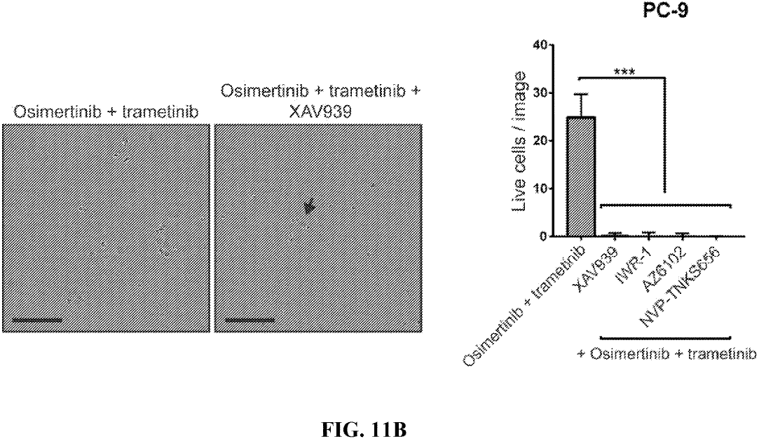

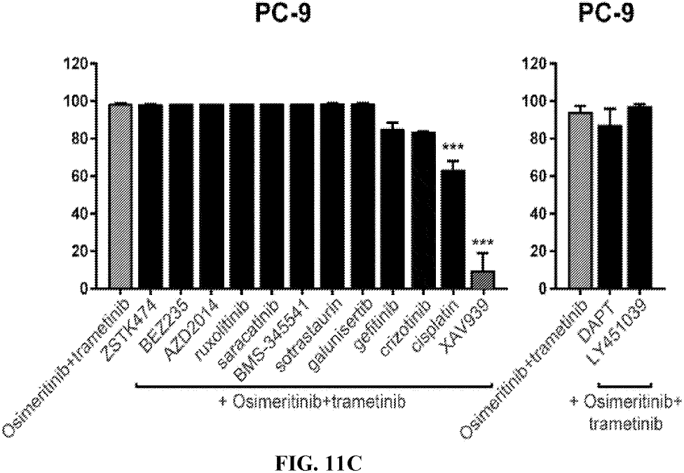

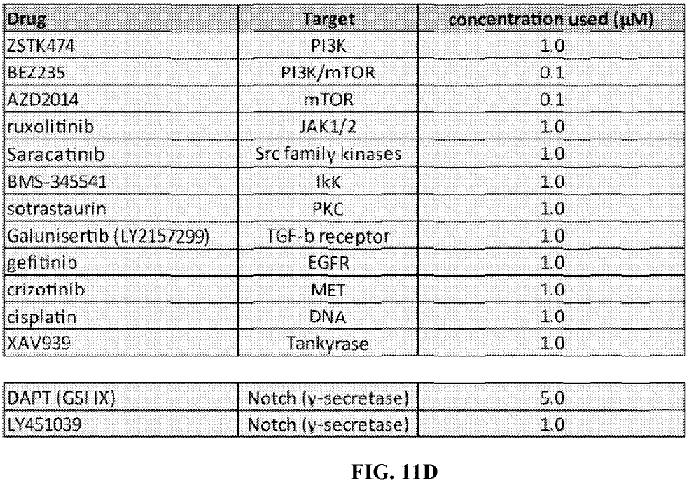

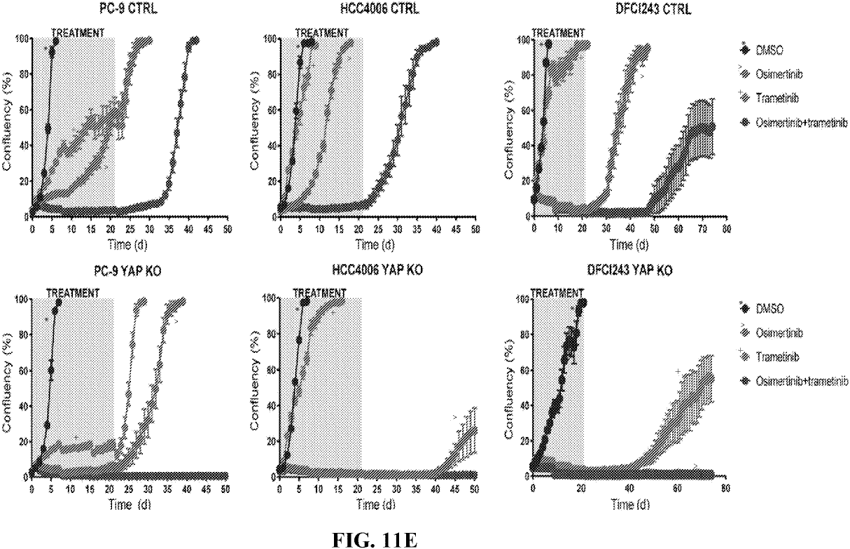

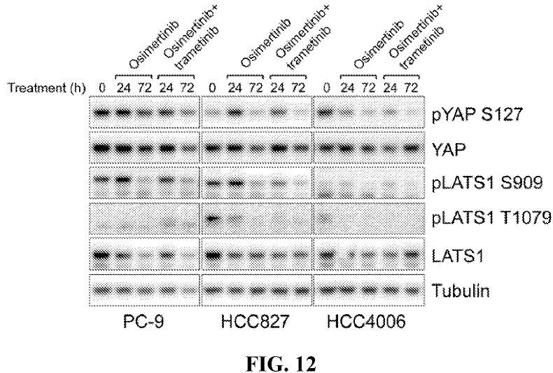

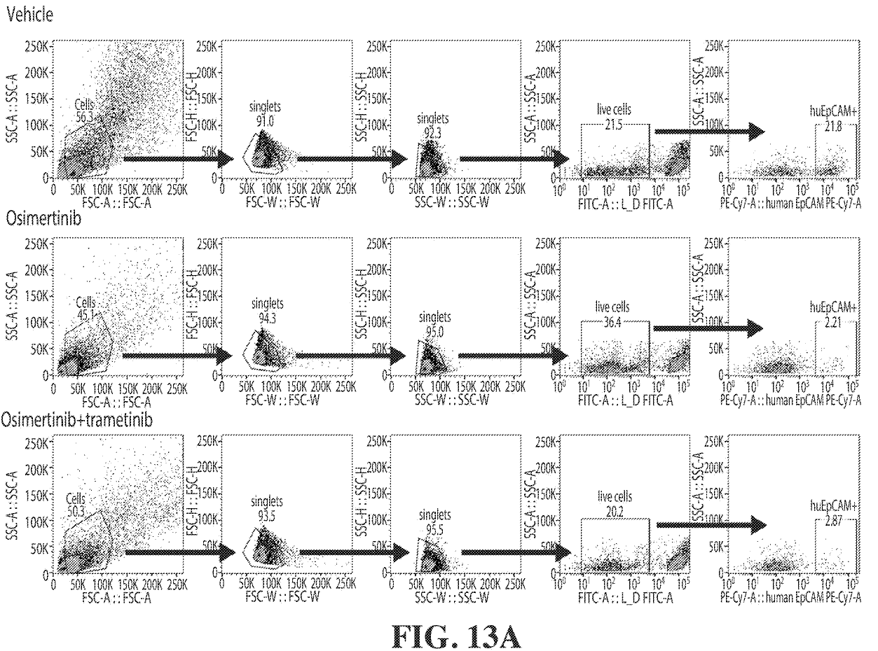

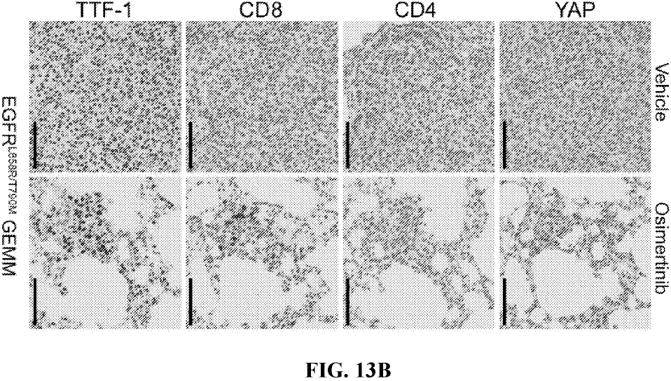

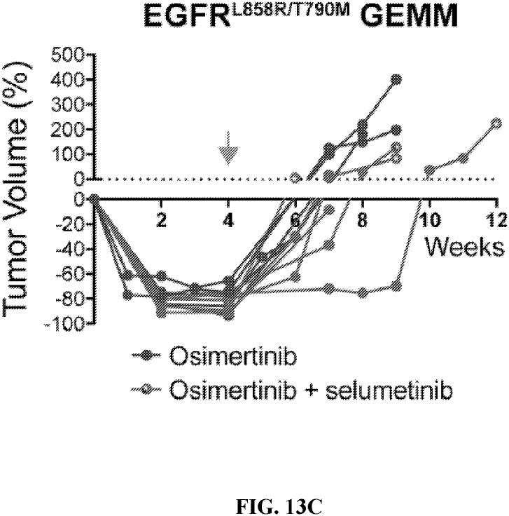

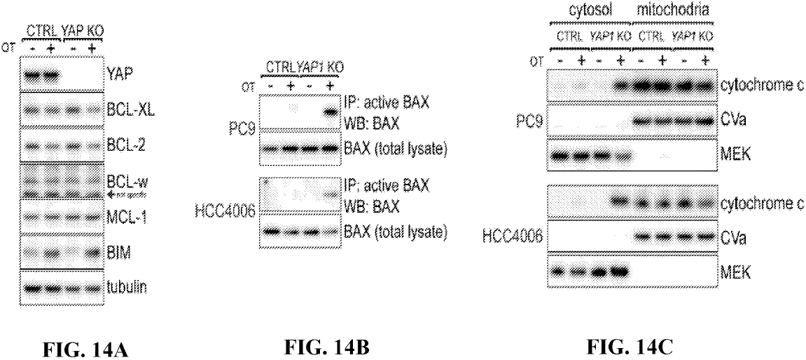

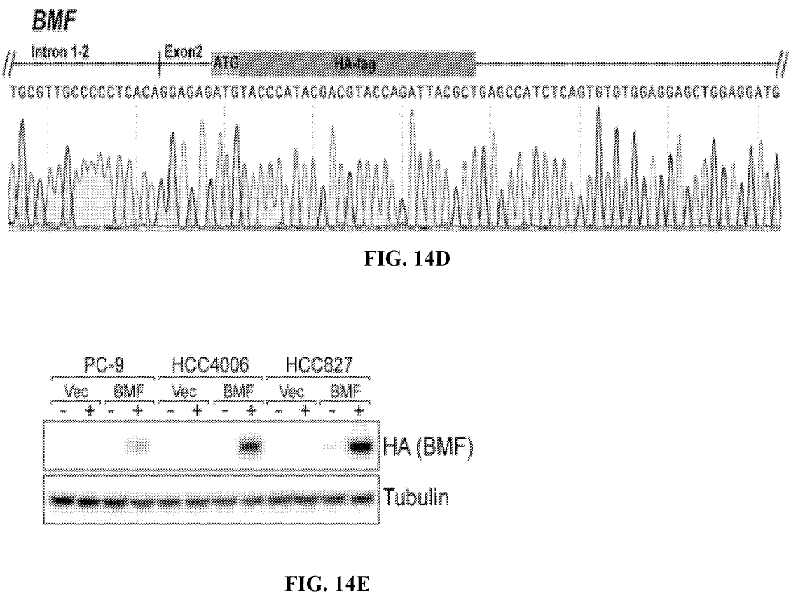

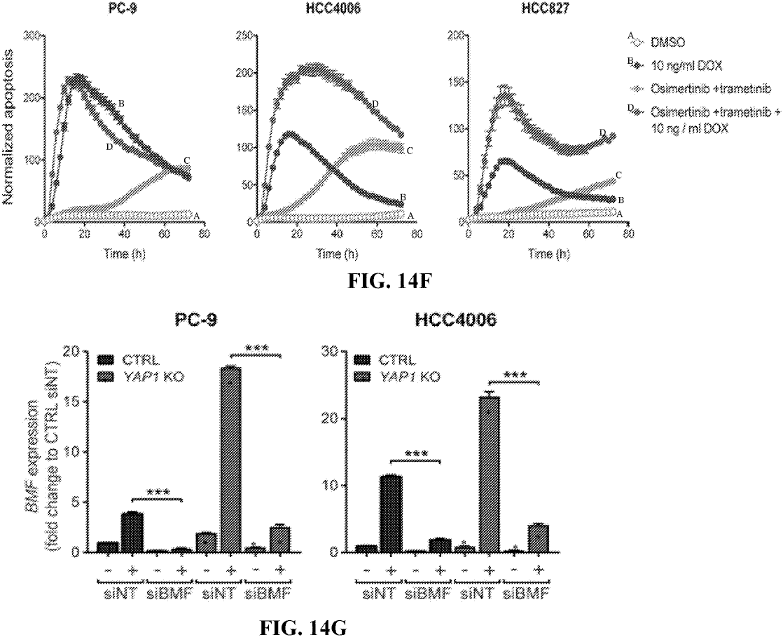

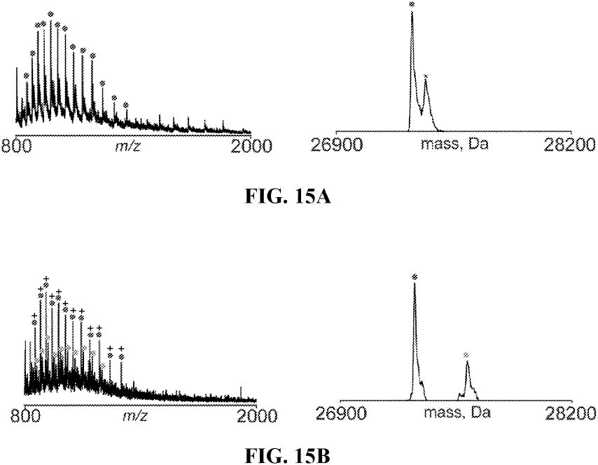

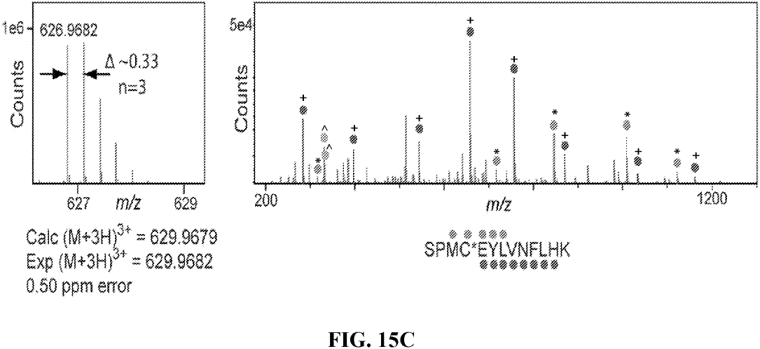

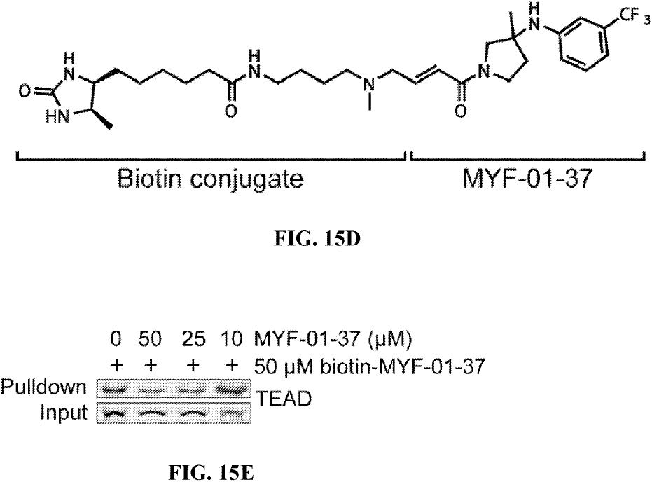

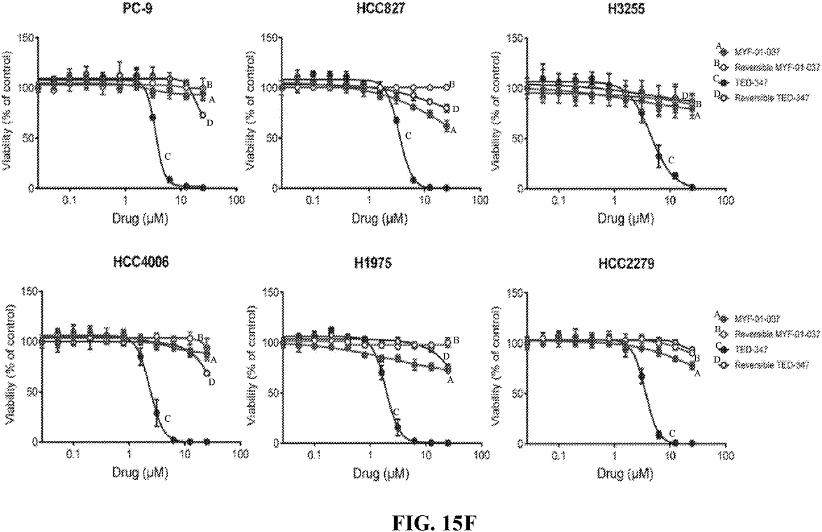

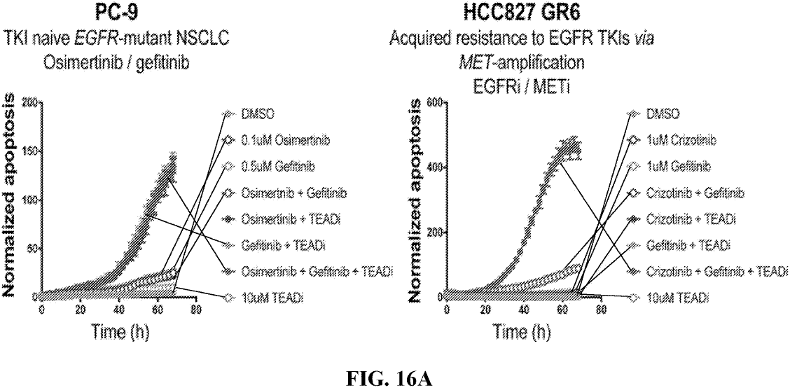

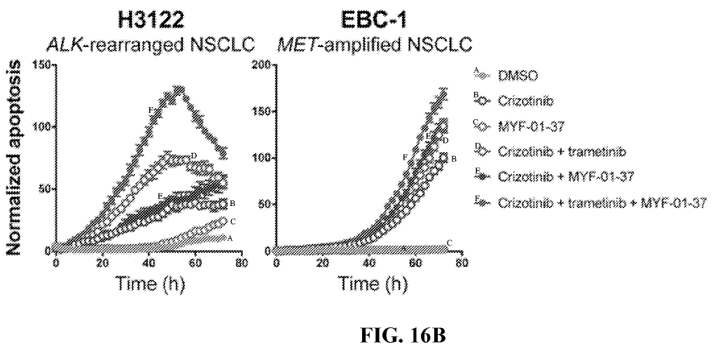

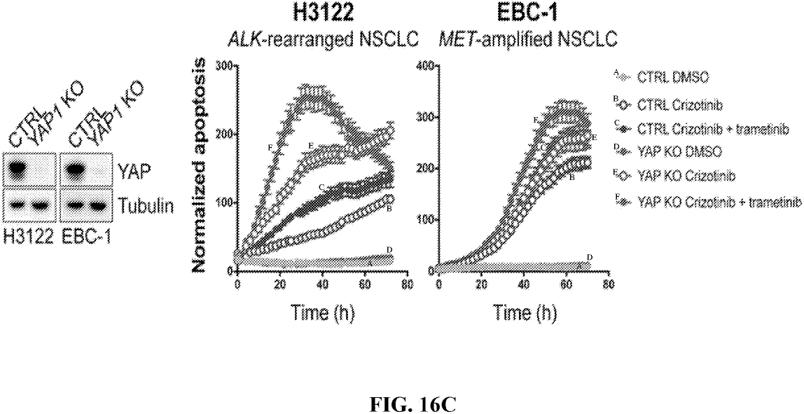

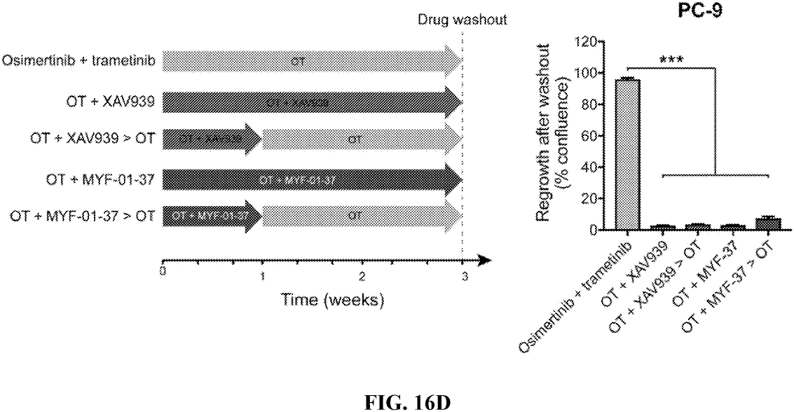

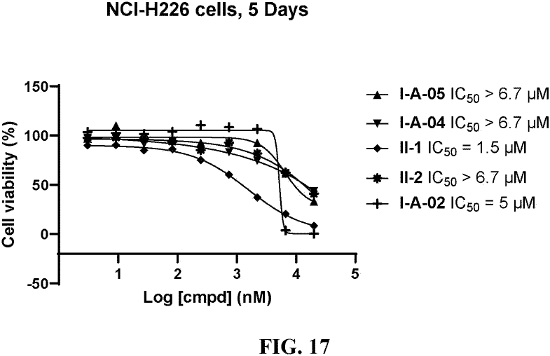

. FIG. 7F shows YAP activity in PC-9 YAP reporter cells after 72 hour treatment with OT or OT in combination with XAV939 (XAV) or MYF-01-37 (MYF). FIG. 7G shows QPCR analysis of BMF expression in cells in FIG. 7E, following 24 hour treatment as indicated. FIG. 7H shows apoptosis in PC-9 and HCC4006 cells treated as indicated. FIG. 7I shows percentage confluence, the regrowth of PC-9 and HCC4006 cells after drug washout following a two-week treatment as indicated. Mean ± SEM are shown in all plots except FIG. 7E, where mean ± SD is shown. ANOVA was used for statistical analyses. ***, P<0.001; **, P<0.01. See also FIGS. 15A-15F.

FIG. 8A shows Western blot analysis of EGFR and ERK phosphorylation in EGFR-mutant NSCLC cell line HCC4006 following treatment with osimertinib alone or in combination with trametinib for indicated times. FIG. 8B shows EGFR-mutant NSCLC cells H1975 and HCC4006 treated as indicated for 6 weeks followed by washout of all drugs. Cell proliferation was monitored manually by weekly determining the proportion of wells >50% confluent. FIG. 8C on the top panel shows: PC-9 cells were grown to -40% confluence and then treated with DMSO or with the combination of osimertinib and trametinib for 21 days, followed by drug washout. The rebounding cells were re-treated with the same drug combination after cells re-entered exponential growth phase (-40% confluence). Arrows indicate the time points for representative images. FIG. 8C on the bottom panel shows representative images of DMSO-treated, dormant, and rebounded PC-9 cells.

FIG. 9A shows barcode abundance plots for osimertinib- and osimertinib/trametinib-treated samples. In FIG. 9A, shared data is shown in darker grey; unique data is shown in lighter grey. FIG. 9B shows the overlap of shared barcodes between osimertinib- and osimertinib/trametinib-treated samples.

FIG. 10A shows GSEA of senescence-associated signature comparing osimertinib and trametinib (OT)-treated, dormant HCC827 and HCC4006 cells vs. DMSO-treated control cells. FIG. 10B shows secreted cytokines/chemokines in the OT-treated dormant cell conditioned media. Log2 fold change (dormant vs. control) is shown. FIG. 10C shows differentially expressed SASP genes in dormant PC-9, HCC827, and HCC4006 cells vs. DMSO-treated control cells. The genes encoding for SASP factors listed in Coppé et al., 2010 and/or included in the luminex panel used in FIG. 10B are shown. Colors indicate log2 fold change values with p<0.05. FIG. 10D shows Western blot analysis of p27^{Kip}, p16^{INK4a}, and p21^{Cip1} protein levels in PC-9, HCC827 and HCC4006 cells following treatment with OT for indicated durations or 21 days followed by drug washout (rebound).

FIG. 11A shows ATAC-seq signal traces at the CTGF locus in PC-9 cells treated for 48 hours with DMSO or for 2 weeks with osimertinib or osimertinib + trametinib. Putative distal enhancer sites upstream of CTGF TSS are highlighted. FIG. 11B shows viable dormant PC-9 cells were manually counted from Incucyte images after 21-day treatment with osimertinib and trametinib (OT) alone or in combination with structurally divergent tankyrase inhibitors. FIG. 11C shows regrowth of PC-9 cells following treatment with osimertinib/trametinib alone or in combination with the indicated drugs for three weeks, followed by drug washout. FIG. 11D is a table showing the targets and the concentrations of the drugs used in the assay. FIG. 11E shows proliferation of YAP1 knock-out (KO) and control (CTRL) cells treated with single-agent osimertinib or with osimertinib/trametinib. Mean ± SD (as in FIG. 11B), or mean ± SEM (as in FIGS. 11C-11D) are shown. ANOVA was used for statistical analyses. ***, P<0.001.

FIG. 12 shows Western blot analysis of YAP and LATS phosphorylation in EGFR-mutant NSCLC cells, in the PC-9 (human non-small cell lung cancer), HCC827 (human lung cancer), and HCC4006 (human non-small cell lung cancer) cell lines, following treatment as indicated.

FIG. 13A shows complete FACS sorting schemes used to obtain scRNA-seq samples from the dissociated PC-9 xenograft tumors. FIG. 13B shows IHC staining for YAP, CD4, CD8, and TTF-1 in residual tumors from EGFR^{L858/T790M} mice following 2-week treatment with vehicle or osimertinib. FIG. 13C shows relative tumor volumes of EGFR^{L858/T790M} treated with osimertinib or osimertinib/selumetinib for four weeks, followed by cessation of treatment (arrow) and follow-up.

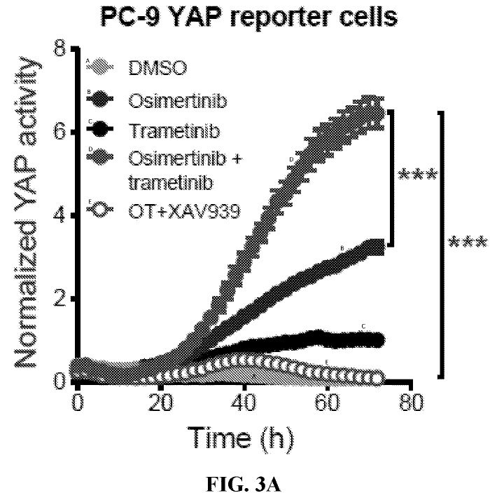

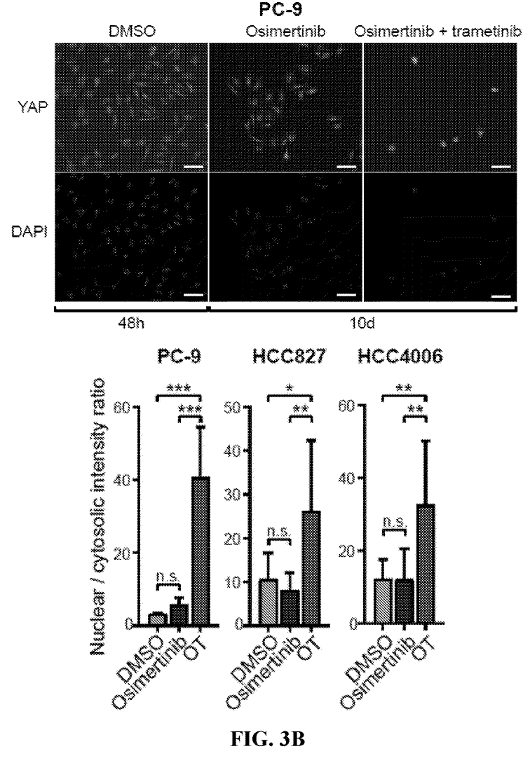

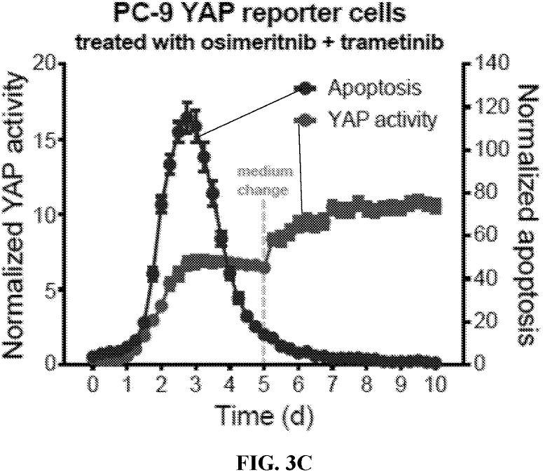

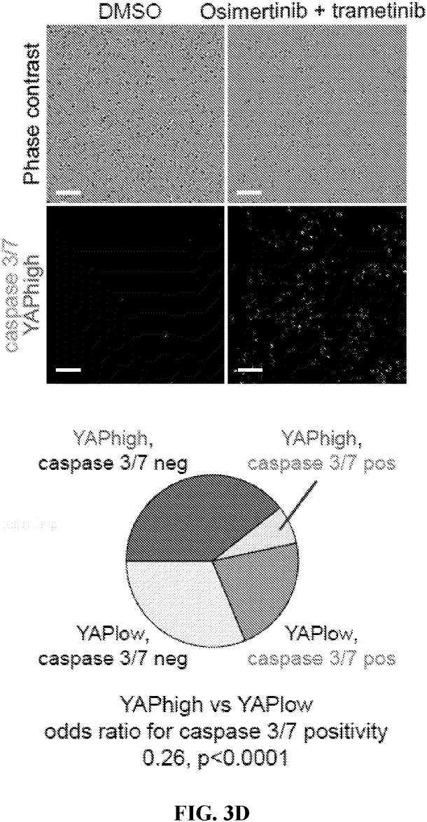

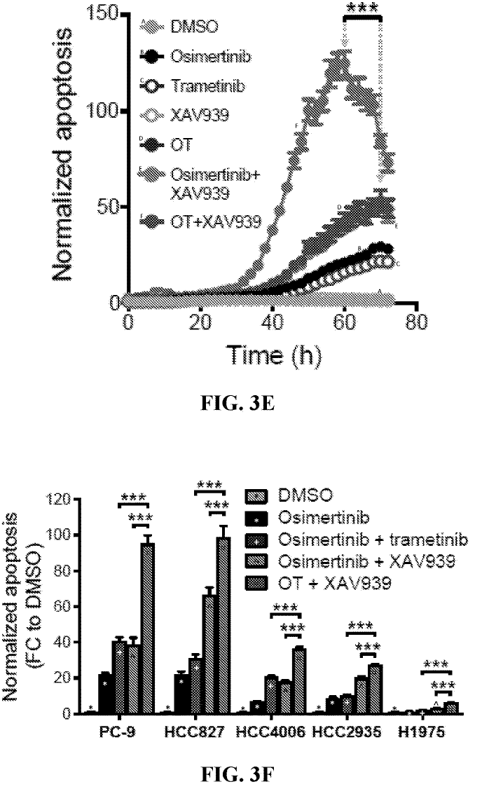

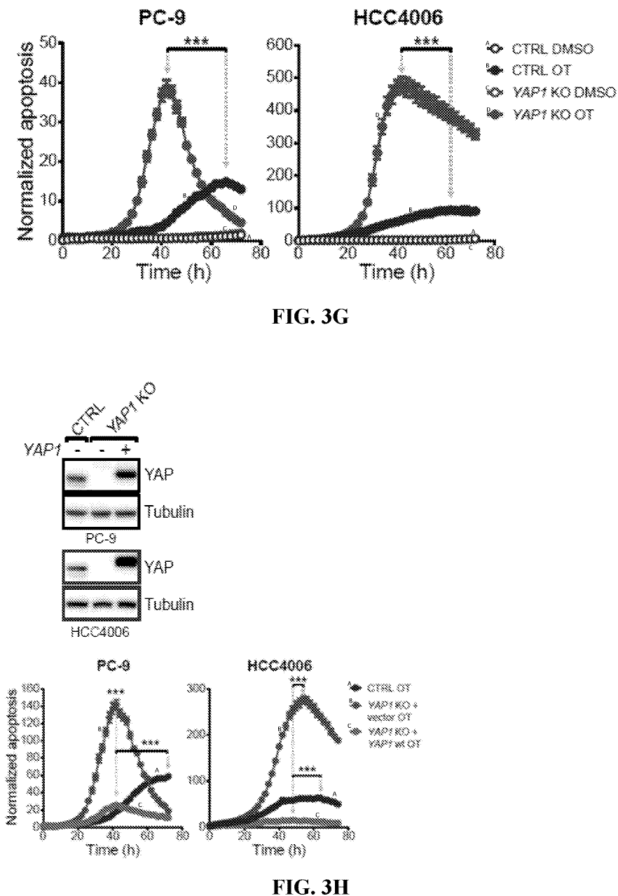

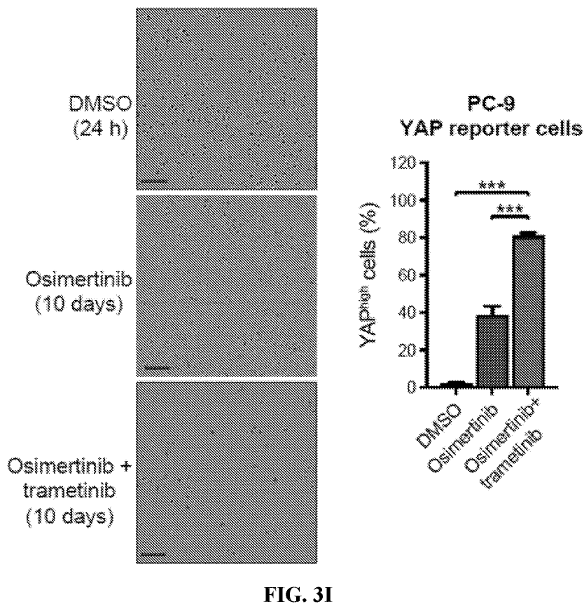

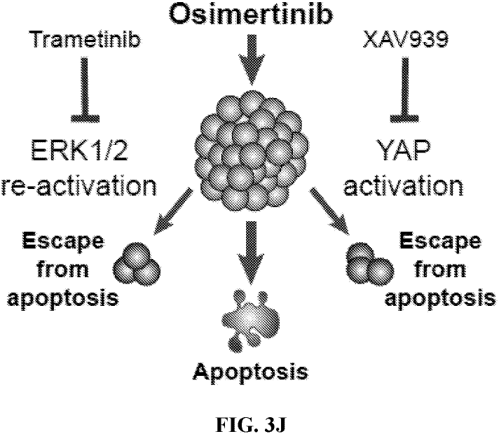

FIG. 14A shows Western blot analysis of indicated protein levels in PC-9 control (CTRL) and YAP1 knock-out (KO) cells following 24 hour treatment with osimertinib and trametinib (OT). FIG. 14B shows cells were treated as in FIG. 14A, and active BAX protein was immunoprecipitated from cell extracts using the conformation-specific BAX antibody. Immunoprecipitated BAX was detected by western blotting using antibody recognizing total BAX protein. FIG. 14C shows cells were treated as in FIG. 14A, fractioned to cytosolic and mitochondrial fractions and cytochrome c levels were detected using western blotting. ATP synthase subunit alpha (CVa) and MEK were used as mitochondrial and cytosolic fraction controls, respectively. FIG. 14D shows a Sanger sequencing trace around the BMF start codon (ATG) in PC-9 HA-BMF single-cell clone used in the study. FIG. 14E shows Western blot analysis of the induction of HA-tagged BMF following 6 h stimulation with 500 ng/ml doxycycline in PC-9, HCC827, and HCC4006 cells stably transfected with doxycycline-inducible, HA-BMF-encoding construct. FIG. 14F shows apoptosis over time in response to indicated treatments in PC-9, HCC827, and HCC4006 cells stably transfected with doxycycline-inducible HA-BMF-encoding construct. Apoptosis was measured using the Incucyte live cell analysis system machine as in FIG. 3D. FIG. 14G shows QPCR analysis of BMF expression following 24 hour treatment with either DMSO or OT in PC-9 and HCC4006 cells transfected with non-targeting (NT) or BMF siRNA. Mean ± SEM (as in FIG. 14F) or mean ± SD (as in FIG. 14G) are shown. ANOVA was used for statistical analyses. ***, P<0.001.

FIGS. 15A-15B show mass spectra (left) and zero charge mass spectra (right) of TEAD2 protein treated with DMSO, as in FIG. 15A, or a 20-fold molar excess of compound MYF-01-37 for 6 hours at 37° C., as in FIG. 15B. Peaks corresponding to unlabeled protein are marked with red (plus sign “+” symbol) glyphs, while MYF-01-37-labeled protein peaks are indicated with green glyphs. FIG. 15C shows MS (left) and MS/MS (right) spectra corresponding to the TEAD2 tryptic peptide ₃₇₇SPMC*EYLVNFLHK³⁸⁹, where C* indicates MYF-01-37 modified cysteine. Ions of type b and y are indicated with blue (asterisk “*” symbol) and red (plus sign “+” symbol) glyphs, respectively. Inhibitor derived thiolated ion is marked with a green (carat “^” symbol) glyph. FIG. 15D shows the structure of a MYF-01-37 biotin conjugate. FIG. 15E shows competition pulldown of TEAD from MDA-MB-231 cell lysates using biotinylated MYF-01-37 after 6 h incubation with indicated concentrations of unlabeled MYF-01-37. FIG. 15F shows dose-response graphs showing the viability in the depicted EGFR-mutant NSCLC cell lines (PC-9, HCC827, H3255, HCC4006, H1975, HCC2279) treated with the depicted compounds (MYF-01-37, TED-347) at the specified concentrations, and the corresponding reversible control compounds lacking the covalent warhead.

FIGS. 16A-16B show apoptosis in NSCLC cell lines treated as indicated. FIG. 16C on the left shows: Western blot analysis of YAP expression in control (CTRL) and YAP1 KO H3122 and EBC-1 cells. FIG. 16C on the right shows: apoptosis in CTRL and YAP1 KO H3122 and EBC-1 cells treated as indicated. FIG. 16D shows PC-9 cells were treated as indicated in the scheme on the left, followed by drug washout. Regrowth of cells was monitored and quantified when confluency in continuously osimertinib/trametinib - treated wells reached >90% confluency. Mean ± SEM are shown. ANOVA was used for statistical analyses. ***, P<0.001.

FIG. 17 shows % cell viability in NCI-H226 human mesothelioma cells, versus the logarithm of the concentration of the indicated compounds (nM) and IC₅₀ values after a 5-day administration of exemplary compounds I-A-05, I-A-04, 11-1, 11-2, and I-A-02 (at the indicated concentrations). The structures of these compounds are shown in Example 1 below.

### DETAILED DESCRIPTION OF CERTAIN EMBODIMENTS OF THE INVENTION

The present disclosure provides inhibitors of a transcription factor (e.g., TEAD, such as TEAD1, TEAD2, TEAD3, TEAD4). In certain embodiments, the inventive compounds inhibit the activity of TEAD (e.g., TEAD1, TEAD2, TEAD3, TEAD4). The present disclosure further provides methods of using the compounds described herein, e.g., as biological probes to study the inhibition of the activity of a transcription factor (e.g., TEAD, such as TEAD1, TEAD2, TEAD3, TEAD4), inhibiting the transcription of a gene (e.g., a gene controlled or regulated by a transcription factor (e.g., TEAD, such as TEAD1, TEAD2, TEAD3, TEAD4)), and as therapeutics, e.g., in the treatment and/or prevention of diseases associated with the overexpression and/or aberrant activity of a transcription factor (e.g., TEAD, such as TEAD1, TEAD2, TEAD3, TEAD4). In certain embodiments, the compounds covalently inhibit TEAD1. In certain embodiments, the compounds covalently inhibit TEAD2. In certain embodiments, the compounds covalently inhibit TEAD3. In certain embodiments, the compounds covalently inhibit TEAD4. In certain embodiments, the diseases treated and/or prevented include, but are not limited to, proliferative diseases, inflammatory diseases, and autoimmune diseases. The proliferative diseases include, but are not limited to, cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma). In certain embodiments, the cancer is a sarcoma (e.g., Kaposi’s sarcoma). In certain embodiments, the cancer is a carcinoma. In certain embodiments, the cancer is lung cancer (e.g., non-small cell lung cancer, mesothelioma). In certain embodiments, the cancer is associated with the overexpression and/or aberrant activity of a transcription factor (e.g., TEAD, such as TEAD1, TEAD2, TEAD3, TEAD4). In certain embodiments, the disease is an inflammatory disease (e.g., fibrosis). In certain embodiments, the disease is an autoimmune disease (e.g., sclerosis). Also provided by the present disclosure are pharmaceutical compositions, kits, methods, and uses of a compound of Formula (I-A), (I-B), or (II), as described herein.

### Compounds

Certain aspects of the present disclosure relate to the compounds described herein, which inhibit the activity of a transcription factor (e.g., TEAD, such as TEAD1, TEAD2, TEAD3, TEAD4). The compounds described herein may be useful in treating and/or preventing diseases (e.g., proliferative diseases (e.g., cancers), inflammatory diseases (e.g., fibrosis), autoimmune diseases (e.g., sclerosis), or diseases associated with the activity of a transcription factor (e.g., TEAD, such as TEAD1, TEAD2, TEAD3, TEAD4)) in a subject, inhibiting the activity of a transcription factor (e.g., TEAD, such as TEAD1, TEAD2, TEAD3, TEAD4), or inhibiting the transcription of a gene (e.g., a gene controlled or regulated by a transcription factor (e.g., TEAD, such as TEAD1, TEAD2, TEAD3, TEAD4)) in a subject or biological sample. In certain embodiments, a compound described herein is a compound of Formula (I-A), (I-B), or (II), or a pharmaceutically acceptable salt, solvate, hydrate, polymorph, co-crystal, tautomer, stereoisomer, isotopically labeled derivative (e.g., deuterated form), prodrug, composition, or mixture thereof. In certain embodiments, a compound described herein is a compound of Formula (I-A), (I-B), or (II), or a pharmaceutically acceptable salt thereof. In certain embodiments, a compound described herein is a compound of Formula (I-A), or a pharmaceutically acceptable salt thereof. In certain embodiments, a compound described herein is a compound of Formula (I-B), or a pharmaceutically acceptable salt thereof. In certain embodiments, a compound described herein is a compound of Formula (II), or a pharmaceutically acceptable salt thereof.

In certain embodiments, a compound described herein is of Formula (I-A):

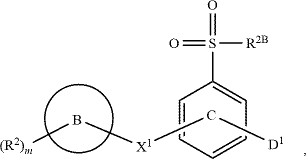

or a pharmaceutically acceptable salt, co-crystal, tautomer, stereoisomer, solvate, hydrate, polymorph, isotopically enriched derivative, or prodrug thereof, wherein:
Ring B is cyclohexyl or phenyl;
R² is halogen, optionally substituted acyl, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, —OR^{c1}, —NO₂, —N(R^{c2})₂, —SR^{c1}, —CN, or —SCN,
wherein R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{c2} are taken together with their intervening atoms to form a substituted or unsubstituted heterocyclic or substituted or unsubstituted heteroaryl ring;
R^{2B} is —N(R^{c2})₂, —OR^{c1}, optionally substituted alkyl, optionally substituted alkenyl, optionally substituted alkynyl, optionally substituted carbocyclyl, optionally substituted heterocyclyl, optionally substituted aryl, or optionally substituted heteroaryl;
X¹ is —O—, —O(alkylene)—, alkylene, —S—, —SCH₂—, —N(R^{da})—, or —N(R^{da})CH₂—;
R^{da} is hydrogen, optionally substituted C₁₋₆ alkyl, optionally substituted acyl, or a nitrogen protecting group;
m is 0, 1, 2, 3, 4, 5, 6, 7, 8, 9, or 10; and
D¹ is a warhead of any one of Formulae (i-1) to (i-23), (i-26) to (i-31), (i-34) to (i-40), (i-42), or (i-43):

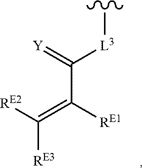

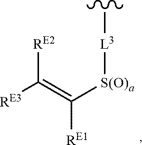

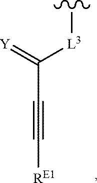

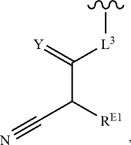

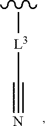

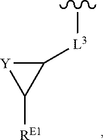

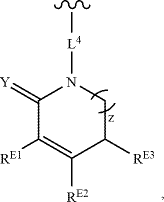

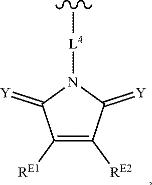

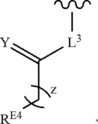

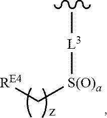

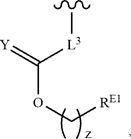

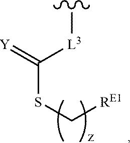

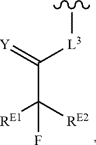

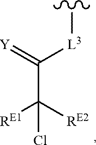

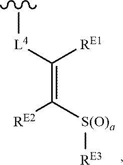

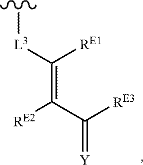

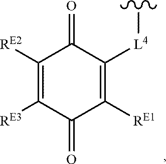

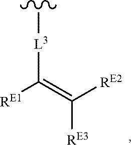

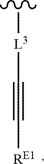

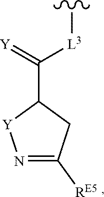

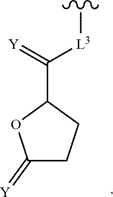

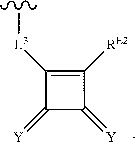

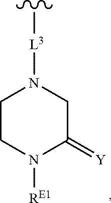

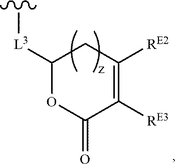

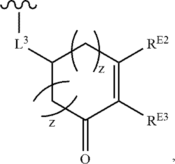

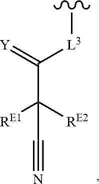

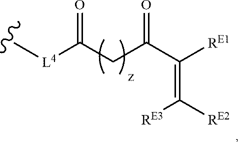

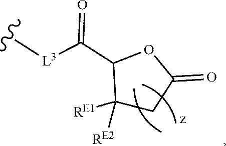

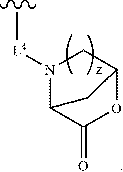

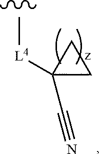

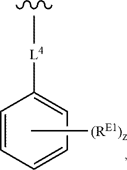

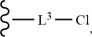

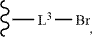

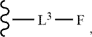

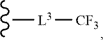

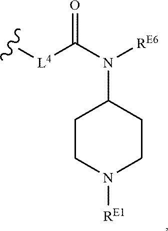

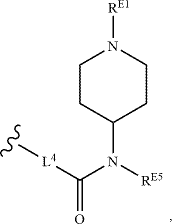

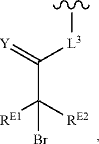

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans—CR^{L3b}═CR^{L3b}—, cis—CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits.

In certain embodiments, a compound described herein is of Formula (I-A):

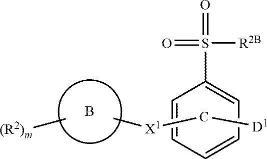

or a pharmaceutically acceptable salt thereof.

In certain embodiments, a compound described herein is of Formula (I-B):

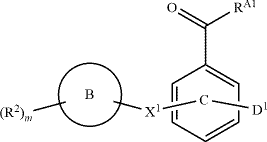

or a pharmaceutically acceptable salt, co-crystal, tautomer, stereoisomer, solvate, hydrate, polymorph, isotopically enriched derivative, or prodrug thereof, wherein:
R^{A1} is —O(R^{a2}) or —N(R^{a3})₂;
R^{a2} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group; and
each instance of R^{a3} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —SO₂(R^{a4}), or a nitrogen protecting group, or optionally two instances of R^{a3} are taken together with their intervening atoms to form a substituted or unsubstituted heterocyclic or substituted or unsubstituted heteroaryl ring; and
R^{a4} is optionally substituted alkyl, optionally substituted alkenyl, optionally substituted alkynyl, optionally substituted carbocyclyl, optionally substituted heterocyclyl, optionally substituted aryl, or optionally substituted heteroaryl;
Ring B is cyclohexyl or phenyl;
R² is halogen, optionally substituted acyl, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, —OR^{c1}, —NO₂, —N(R^{c2})₂, —SR^{c1}, —CN, or —SCN,
wherein R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{c2} are taken together with their intervening atoms to form a substituted or unsubstituted heterocyclic or substituted or unsubstituted heteroaryl ring;
X¹ is —O—, —O(alkylene)—, alkylene, —S—, —SCH₂—, —N(R^{da})—, or —N(R^{da})CH₂—;
R^{da} is hydrogen, optionally substituted C₁₋₆ alkyl, or a nitrogen protecting group;
m is 0, 1, 2, 3, 4, 5, 6, 7, 8, 9, or 10; and
D¹ is a warhead of any one of Formulae (i-1) to (i-23), (i-26) to (i-31), (i-34) to (i-40), (i-42), or (i-43):

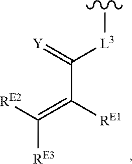

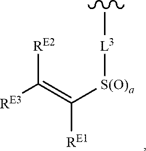

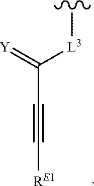

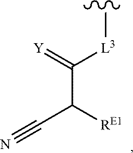

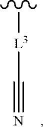

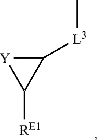

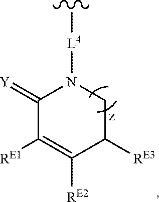

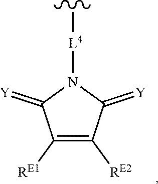

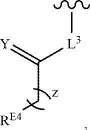

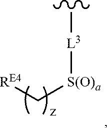

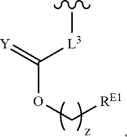

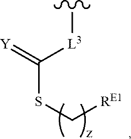

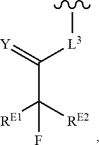

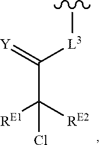

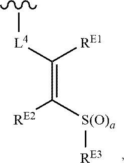

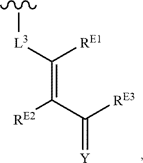

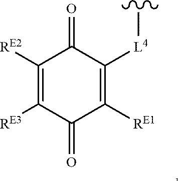

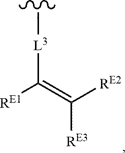

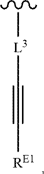

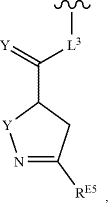

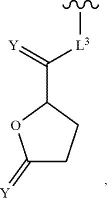

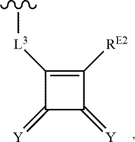

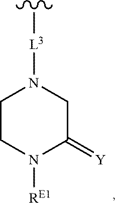

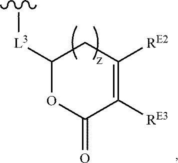

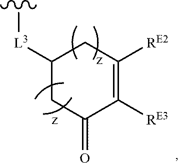

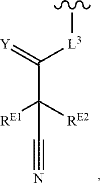

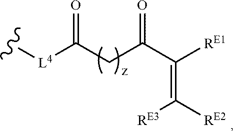

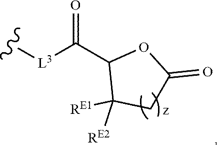

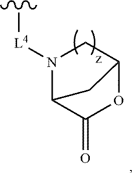

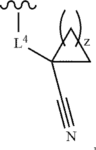

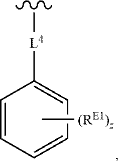

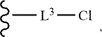

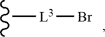

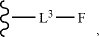

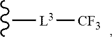

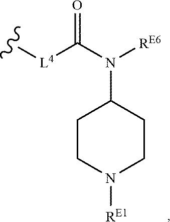

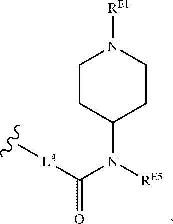

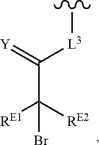

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans—CR^{L3b}═CR^{L3b}—, cis—CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits;
provided that the compound is not of formula:

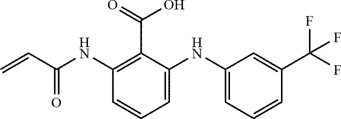 [SMILES: C=CC(=O)Nc1cccc(Nc2cccc(C(F)(F)F)c2)c1C(=O)O]

or

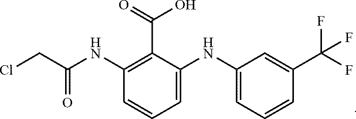

In certain embodiments, a compound described herein is of Formula (I-B):

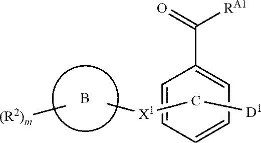

or a pharmaceutically acceptable salt thereof.

In certain embodiments, a compound described herein is of Formula (II):

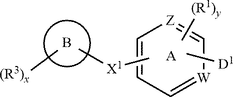

or a pharmaceutically acceptable salt, co-crystal, tautomer, stereoisomer, solvate, hydrate, polymorph, isotopically enriched derivative, or prodrug thereof, wherein:
Ring B is cyclohexyl or phenyl;
W is —C(R^{a})═ or —N═, as valency permits; and R^{a} is hydrogen, halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN;
Z is —C(R^{b})═ or —N═, as valency permits; and R^{b} is hydrogen, halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN;
provided at least one instance of W and Z is —C(R^{a})═ or —C(R^{b})═;
each instance of R¹ is independently halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN;
each instance of R³ is independently halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN;
wherein R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{c2} are taken together with their intervening atoms to form a substituted or unsubstituted heterocyclic or substituted or unsubstituted heteroaryl ring;
X¹ is —O—, —O(alkylene)—, alkylene, —S—, —SCH₂—, —N(R^{da})—, or —N(R^{da})CH₂—;
R^{da} is hydrogen, optionally substituted C₁₋₆ alkyl, optionally substituted acyl, or a nitrogen protecting group;
x is 0, 1, 2, 3, 4, 5, 6, 7, 8, 9, or 10;
y is 0, 1, 2, 3, or 4;
D¹ is a warhead of any one of Formulae (i-1) to (i-23), (i-26) to (i-31), (i-34) to (i-40), (i-42), or (i-43):

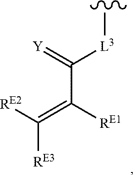

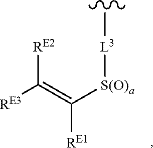

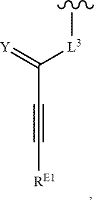

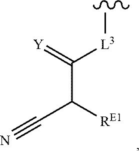

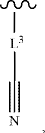

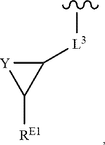

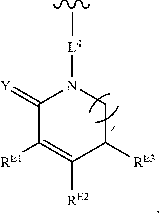

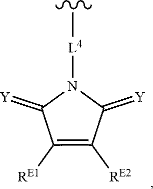

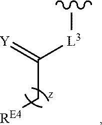

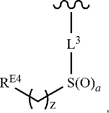

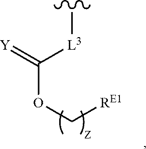

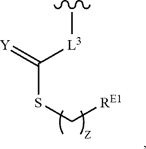

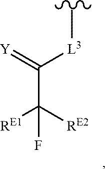

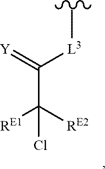

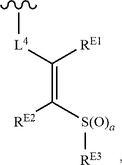

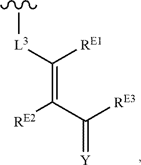

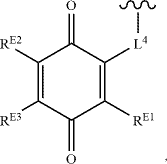

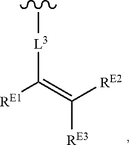

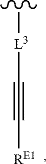

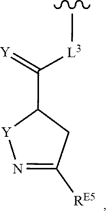

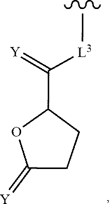

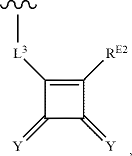

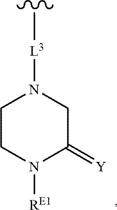

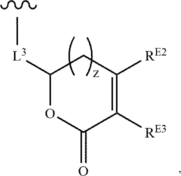

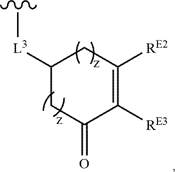

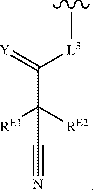

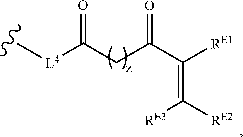

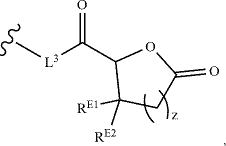

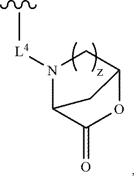

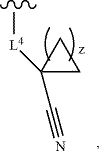

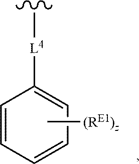

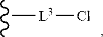

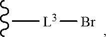

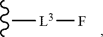

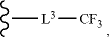

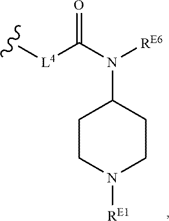

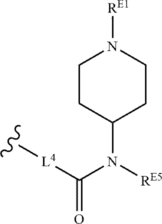

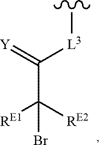

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}—, —SC(═O)—, —C(═O)S—, —OC(═O)—, —C(═O)O—, —NR^{L3a}C(═S)—, —C(═S)NR^{L3a}—, trans—CR^{L3b}═CR^{L3b}—, cis—CR^{L3b}═CR^{L3b}—, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)₂—, —S(═O)₂NR^{L3a}—, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits;
provided that the compound is not of formula:

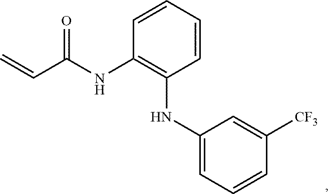

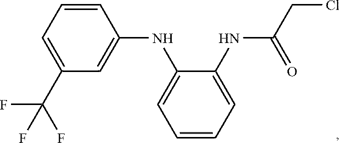

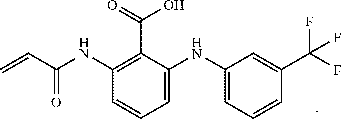

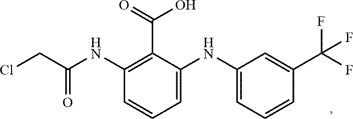

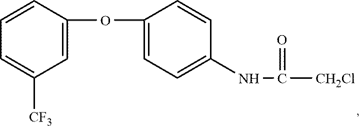

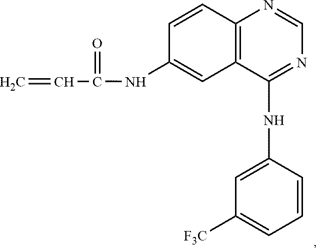

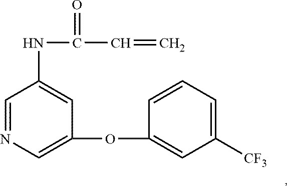

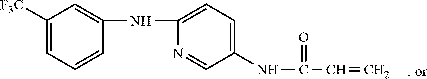

or

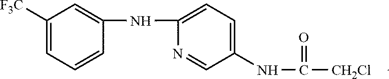

In certain embodiments, a compound described herein is of Formula (II):

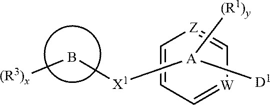

or a pharmaceutically acceptable salt thereof.

### Ring B

Formulae (I-A), (I-B), and (II) include Ring B.

### Ring B (Formulae (I-A) and (I-B))

In certain embodiments, in a compound of Formula (I-A) or (I-B), there are zero instances of substituent R² on Ring B. In certain embodiments, in a compound of Formula (I-A) or (I-B), there are one or more instances of substituent R² on Ring B. In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, at least one instance of R² is halogen (e.g., F, Cl, Br, or I). In certain embodiments, at least one instance of R² is optionally substituted acyl (e.g., —C(═O)Me). In certain embodiments, at least one instance of R² is optionally substituted alkyl (e.g., substituted or unsubstituted C₁₋₆ alkyl). In certain embodiments, at least one instance of R² is alkyl optionally substituted with halogen. In certain embodiments, at least one instance of R² is optionally substituted C₁₋₆ alkyl. In certain embodiments, at least one instance of R² is C₁₋₆ alkyl optionally substituted with halogen. In certain embodiments, at least one instance of R² is —CF₃. In certain embodiments, at least one instance of R² is substituted or unsubstituted methyl. In certain embodiments, at least one instance of R² is substituted or unsubstituted ethyl. In certain embodiments, at least one instance of R² is substituted or unsubstituted propyl. In certain embodiments, at least one instance of R² is optionally substituted alkenyl (e.g., substituted or unsubstituted C₂₋₆ alkenyl). In certain embodiments, at least one instance of R² is optionally substituted alkynyl (e.g., substituted or unsubstituted C₂₋₆ alkynyl). In certain embodiments, at least one instance of R² is optionally substituted carbocyclyl (e.g., substituted or unsubstituted, 3- to 10-membered, monocyclic carbocyclyl comprising zero, one, or two double bonds in the carbocyclic ring system). In certain embodiments, at least one instance of R² is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R² is optionally substituted aryl (e.g., substituted or unsubstituted, 6- to 10-membered aryl). In certain embodiments, at least one instance of R² is benzyl. In certain embodiments, at least one instance of R² is substituted or unsubstituted phenyl. In certain embodiments, at least one instance of R² is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R² is —OR^{c1} (e.g., —OH or —OMe). In certain embodiments, at least one instance of R² is —N(R^{c2})₂ (e.g., —NMe₂). In certain embodiments, at least one instance of R² is —SR^{c1} (e.g., —SMe). In certain embodiments, at least one instance of R² is —NO₂. In certain embodiments, at least one instance of R² is —CN. In certain embodiments, at least one instance of R² is —SCN. In certain embodiments, m is 0 or 1; and R² is optionally substituted alkyl. In certain embodiments, m is 1; and R² is optionally substituted alkyl. In certain embodiments, m is 0 or 1; and R² is optionally substituted C₁₋₆ alkyl. In certain embodiments, m is 1; and R² is optionally substituted C₁₋₆ alkyl. In certain embodiments, m is 0 or 1; and R² is C₁₋₆ alkyl optionally substituted with halogen. In certain embodiments, m is 1; and R² is C₁₋₆ alkyl optionally substituted with halogen. In certain embodiments, m is 0 or 1; and R² is —CF₃. In certain embodiments, m is 1; and R² is —CF₃.

In certain embodiments, at least one instance of R² is —OR^{c1}, —N(R^{c2})₂, or —SR^{c1}, and R ^{c1} and R ^{c2} are as defined herein.

### R^{c1} and R^{c2} (Formulae (I-A), (I-B), and (II))

Formulae (I-A), (I-B), and (II) include R^{c1} and R^{c2} as described herein. In certain embodiments, in Formulae (I-A) or (I-B), at least one instance of R² attached to Ring B is —OR^{c1}, —N(R^{c2})₂, or —SR^{c1}, and R^{c1} and R^{c2} are as defined herein. In certain embodiments, in Formula (II), at least one instance of R³ attached to Ring A is —OR^{c1}, —N(R^{c2})₂, or —SR^{c1}, and R^{c1} and R^{c2} are as defined herein. In certain embodiments, in Formula (II), at least one instance of R¹ attached to Ring A is —OR^{c1}, —N(R^{c2})₂, or —SR^{c1}, and R^{c1} and R^{c2} are as defined herein. In certain embodiments, in Formula (II), substituent Z or W within Ring A is -OR^{c1}, —N(R^{c2})₂, or —SR^{c1}, and R^{c1} and R^{c2} are as defined herein. In certain embodiments, R^{c1} is hydrogen. In certain embodiments, R^{c1} is optionally substituted acyl (e.g., —C(═O)Me. In certain embodiments, R^{c1} is optionally substituted alkyl (e.g., substituted or unsubstituted C₁₋₆ alkyl). In certain embodiments, R^{c1} is substituted or unsubstituted methyl. In certain embodiments, R^{c1} is substituted or unsubstituted ethyl. In certain embodiments, R^{c1} is substituted or unsubstituted propyl. In certain embodiments, R^{c1} is optionally substituted alkenyl (e.g., substituted or unsubstituted C₂₋₆ alkenyl). In certain embodiments, R^{c1} is optionally substituted alkynyl (e.g., substituted or unsubstituted C₂₋₆ alkynyl). In certain embodiments, R^{c1} is optionally substituted carbocyclyl (e.g., substituted or unsubstituted, 3-to 10-membered, monocyclic carbocyclyl comprising zero, one, or two double bonds in the carbocyclic ring system). In certain embodiments, R^{c1} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{c1} is optionally substituted aryl (e.g., substituted or unsubstituted, 6- to 10-membered aryl). In certain embodiments, R^{c1} is benzyl. In certain embodiments, R^{c1} is substituted or unsubstituted phenyl. In certain embodiments, R^{c1} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{c1} is an oxygen protecting group when attached to an oxygen atom. In certain embodiments, R^{c1} is a sulfur protecting group when attached to a sulfur atom.

In certain embodiments, at least one instance of R^{c2} is hydrogen. In certain embodiments, at least one instance of R^{c2} is optionally substituted acyl (e.g., —C(═O)Me). In certain embodiments, at least one instance of R ^{c2} is optionally substituted alkyl (e.g., substituted or unsubstituted C₁₋₆ alkyl). In certain embodiments, at least one instance of R^{c2} is substituted or unsubstituted methyl. In certain embodiments, at least one instance of R^{c2} is substituted or unsubstituted ethyl. In certain embodiments, at least one instance of R^{c2} is substituted or unsubstituted propyl. In certain embodiments, at least one instance of R^{c2} is optionally substituted alkenyl (e.g., substituted or unsubstituted C₂₋₆ alkenyl). In certain embodiments, at least one instance of R^{c2} is optionally substituted alkynyl (e.g., substituted or unsubstituted C₂₋₆ alkynyl). In certain embodiments, at least one instance of R^{c2} is optionally substituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{c2} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{c2} is optionally substituted aryl (e.g., substituted or unsubstituted, 6- to 10-membered aryl). In certain embodiments, at least one instance of R^{c2} is benzyl. In certain embodiments, at least one instance of R^{c2} is substituted or unsubstituted phenyl. In certain embodiments, at least one instance of R^{c2} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{c2} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{c2} are taken together with their intervening atoms to form a substituted or unsubstituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or substituted or unsubstituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

### Ring B (Formulae (I-A) and (I-B))

In certain embodiments, for Formulae (I-A) and (I-B), Ring B is phenyl. In certain embodiments, Ring B is phenyl substituted with one or more instances of substituent R². In certain embodiments, Ring B is of formula:

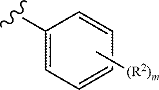

. In certain embodiments, Ring B is of formula:

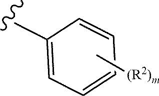

and m is 0 or 1. In certain embodiments, Ring B is of formula:

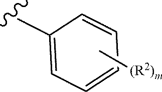

and m is 0, 1, 2, or 3. In certain embodiments, Ring B is of formula:

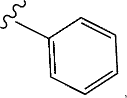

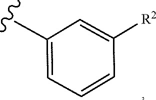

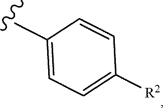

or

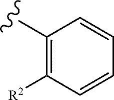

. In certain embodiments, Ring B is of formula:

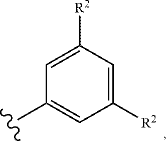

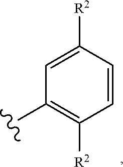

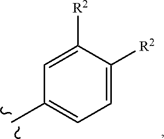

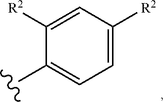

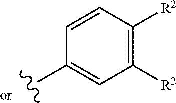

. In certain embodiments, Ring B is of formula:

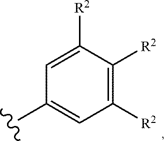

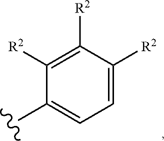

or

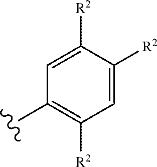

. In certain embodiments, Ring B is of formula:

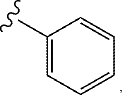

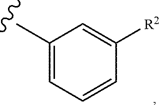

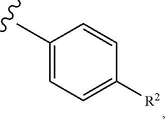

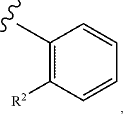

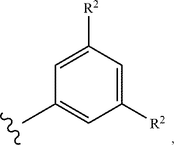

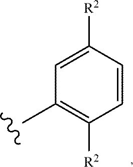

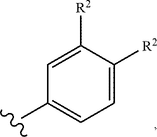

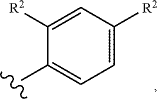

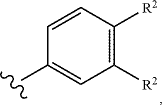

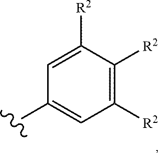

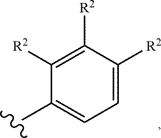

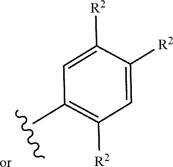

. In certain embodiments, Ring B is of formula:

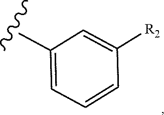

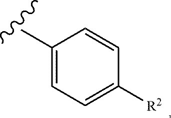

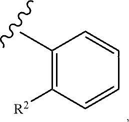

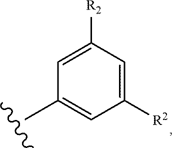

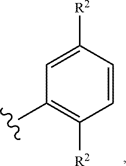

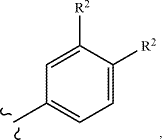

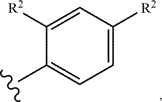

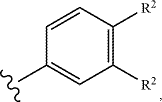

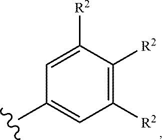

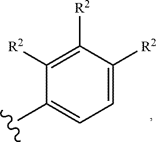

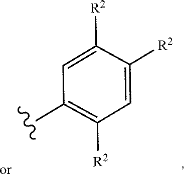

wherein R² is halogen, optionally substituted acyl, optionally substituted alkyl, optionally substituted heteroalkyl, optionally substituted alkenyl, or optionally substituted alkynyl. In certain embodiments, Ring B is of formula:

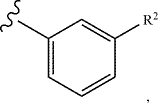

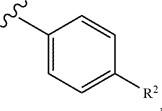

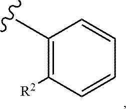

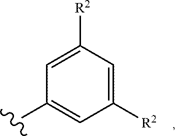

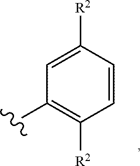

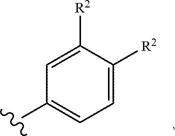

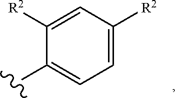

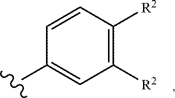

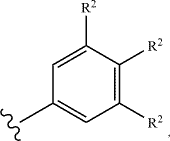

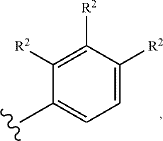

or

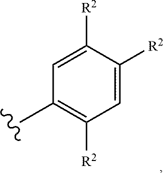

wherein R² is halogen, optionally substituted acyl, or optionally substituted alkyl. In certain embodiments, Ring B is of formula:

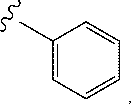

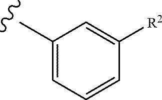

or

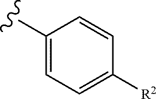

. In certain embodiments, Ring B is of formula:

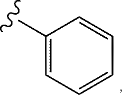

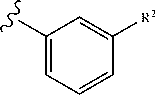

or

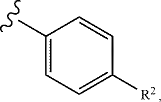

wherein R² is halogen, optionally substituted acyl, or alkyl optionally substituted with halogen. In certain embodiments, the moiety

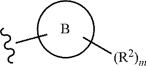

is of formula:

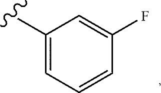

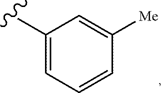

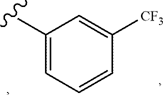

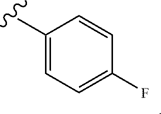

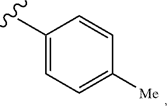

or

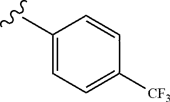

. In certain embodiments, the moiety

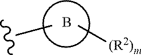

is of formula:

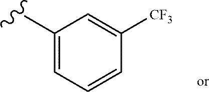

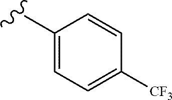

. In certain embodiments, Ring B is of formula:

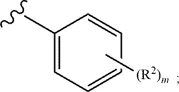

m is 0 or 1; and R² is optionally substituted alkyl or halogen. In certain embodiments, Ring B is of formula:

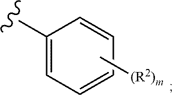

m is 0 or 1; and R² is optionally substituted alkyl. In certain embodiments, Ring B is of formula:

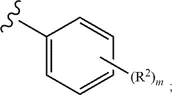

m is 1; and R² is optionally substituted alkyl. In certain embodiments, Ring B is of formula:

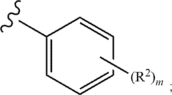

m is 0 or 1; and R² is optionally substituted C₁₋₆ alkyl. In certain embodiments, Ring B is of formula:

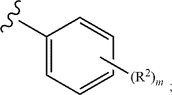

m is 1; and R² is optionally substituted C₁₋₆ alkyl. In certain embodiments, Ring B is of formula:

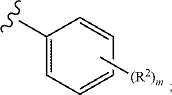

m is 0 or 1; and R² is C₁₋₆ alkyl optionally substituted with halogen. In certain embodiments, Ring B is of formula:

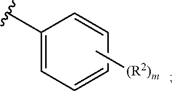

m is 1; and R² is C₁₋₆ alkyl optionally substituted with halogen. In certain embodiments, Ring B is of formula:

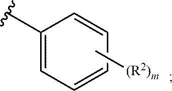

m is 0 or 1; and R² is —CF₃. In certain embodiments, Ring B is phenyl or cyclohexyl, m is 1; and R² is —CF₃.

In certain embodiments, Ring B is cyclohexyl. In certain embodiments, Ring B is cyclohexyl substituted with one or more instances of substituent R². In certain embodiments, Ring B is of formula:

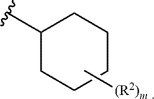

. In certain embodiments, Ring B is of formula:

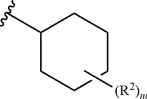

and m is 0 or 1. In certain embodiments, Ring B is of formula:

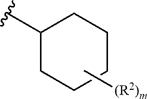

and m is 0, 1, 2, or 3. In certain embodiments, Ring B is of formula:

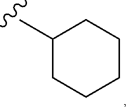

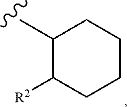

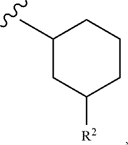

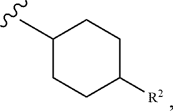

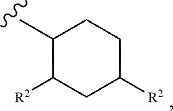

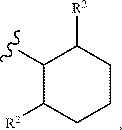

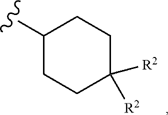

or

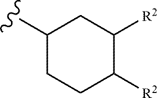

. In certain embodiments, Ring B is of formula:

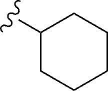

. In certain embodiments, Ring B is of formula:

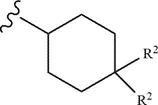

. In certain embodiments, the moiety

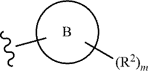

is of formula:

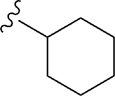

. In certain embodiments, the moiety

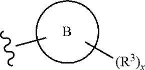

is of formula:

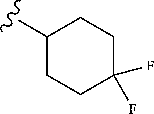

. In certain embodiments, the moiety

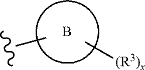

is of formula:

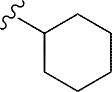

or

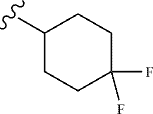

. In certain embodiments, Ring B is of formula:

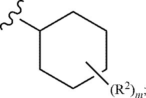

m is 0 or 1; and R² is halogen or optionally substituted alkyl. In certain embodiments, Ring B is of formula:

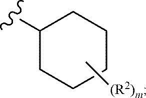

m is 1; and R² is optionally substituted alkyl. In certain embodiments, Ring B is of formula:

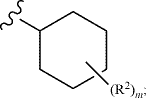

m is 0 or 1; and R² is optionally substituted C₁₋₆ alkyl. In certain embodiments, Ring B is of formula:

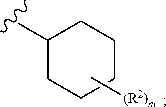

m is 1; and R² is optionally substituted C₁₋₆ alkyl. In certain embodiments, Ring B is of formula:

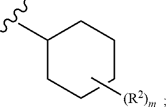

m is 0 or 1; and R² is halogen or C₁₋₆ alkyl optionally substituted with halogen. In certain embodiments, Ring B is of formula:

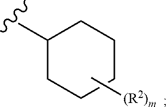

m is 1; and R² is C₁₋₆ alkyl optionally substituted with halogen. In certain embodiments, Ring B is of formula:

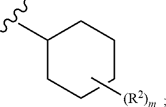

m is 0 or 1; and R² is —F, —Me, —CF₃. In certain embodiments, Ring B is of formula:

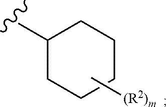

m is 0 or 1; and R² is —CF₃. In certain embodiments, Ring B is of formula:

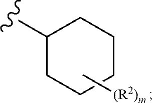

m is 1; and R² is —CF₃.

In certain embodiments, Ring B is of formula:

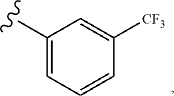

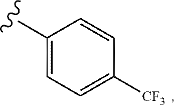

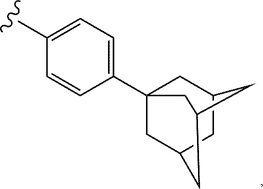

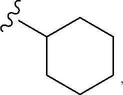

or

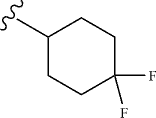

. In certain embodiments, in compounds of Formula (I-A) or (I-B), Ring B is of formula:

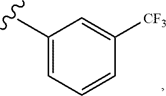

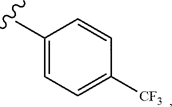

or

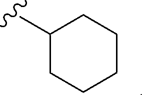

### Ring B (Formula II)

In certain embodiments, in a compound of Formula (II), there are zero instances of substituent R³ on Ring B. In certain embodiments, there are one or more instances of substituent R³ on Ring B. In certain embodiments, x is 0. In certain embodiments, x is 1. In certain embodiments, x is 2. In certain embodiments, x is 3. In certain embodiments, x is 4. In certain embodiments, x is 5. In certain embodiments, x is 6. In certain embodiments, x is 7. In certain embodiments, x is 8. In certain embodiments, x is 9. In certain embodiments, x is 10. In certain embodiments, at least one instance of R³ is halogen (e.g., F, Cl, Br, or I). In certain embodiments, at least one instance of R³ is —F. In certain embodiments, x is 2; and both instances of R³ are halogen (e.g., F, Cl, Br, or I). In certain embodiments, at least one instance of R³ is —Br. In certain embodiments, at least one instance of R³ is —F. In certain embodiments, x is 2; and both instances of R³ are —F. In certain embodiments, at least one instance of R³ is —I. In certain embodiments, at least one instance of R³ is optionally substituted acyl (e.g., —C(═O)Me). In certain embodiments, at least one instance of R³ is optionally substituted alkyl (e.g., substituted or unsubstituted C₁₋₆ alkyl). In certain embodiments, at least one instance of R³ is alkyl optionally substituted with halogen. In certain embodiments, at least one instance of R³ is optionally substituted C₁₋₆ alkyl. In certain embodiments, at least one instance of R³ is C₁₋₆ alkyl optionally substituted with halogen. In certain embodiments, at least one instance of R³ is —CF₃. In certain embodiments, at least one instance of R³ is substituted or unsubstituted methyl. In certain embodiments, at least one instance of R³ is substituted or unsubstituted ethyl. In certain embodiments, at least one instance of R³ is substituted or unsubstituted propyl. In certain embodiments, at least one instance of R³ is optionally substituted alkenyl (e.g., substituted or unsubstituted C₂₋₆ alkenyl). In certain embodiments, at least one instance of R³ is optionally substituted alkynyl (e.g., substituted or unsubstituted C₂₋₆ alkynyl). In certain embodiments, at least one instance of R³ is optionally substituted carbocyclyl (e.g., substituted or unsubstituted, 3- to 14-membered, monocyclic carbocyclyl comprising zero, one, or two double bonds in the carbocyclic ring system). In certain embodiments, at least one instance of R³ is optionally substituted carbocyclyl. In certain embodiments, at least one instance of R³ is optionally substituted C₃₋₁₄ carbocyclyl. In certain embodiments, at least one instance of R³ is optionally substituted C₃₋₁₀ carbocyclyl. In certain embodiments, at least one instance of R³ is optionally substituted adamantyl. In certain embodiments, at least one instance of R³ is optionally substituted C₃₋₇ carbocyclyl. In certain embodiments, at least one instance of R³ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R³ is optionally substituted aryl (e.g., substituted or unsubstituted, 6- to 10-membered aryl). In certain embodiments, at least one instance of R³ is benzyl. In certain embodiments, at least one instance of R³ is substituted or unsubstituted phenyl. In certain embodiments, at least one instance of R³ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R³ is -OR^{c1} (e.g., —OH or —OMe). In certain embodiments, at least one instance of R³ is -N(R^{c2})₂ (e.g., -NMe₂). In certain embodiments, at least one instance of R³ is -SR^{c1} (e.g., -SMe). In certain embodiments, at least one instance of R³ is —NO₂. In certain embodiments, at least one instance of R³ is —CN. In certain embodiments, at least one instance of R³ is —SCN. In certain embodiments, x is 1 or 2; and R³ is optionally substituted alkyl. In certain embodiments, x is 1 or 2; and R³ is halogen, optionally substituted alkyl, or optionally substituted carbocyclyl. In certain embodiments, x is 1 or 2; and R³ is halogen, optionally substituted C₁₋₆ alkyl, optionally substituted C₃₋₁₄ carbocyclyl. In certain embodiments, x is 1 or 2; and R³ is optionally substituted C₁₋₆ alkyl. In certain embodiments, x is 1 or 2; and R³ is halogen, C₁₋₆ alkyl optionally substituted with halogen, or optionally substituted C₃₋₁₄ carbocyclyl. In certain embodiments, x is 1; and R³ is C₁₋₆ alkyl optionally substituted with halogen. In certain embodiments, x is 1 or 2; and R³ is —CF₃, —F, or optionally substituted adamantyl. In certain embodiments, x is 1 or 2; and R³ is —CF₃.

In certain embodiments, Ring B is phenyl. In certain embodiments, Ring B is phenyl substituted with one or more instances of substituent R³. In certain embodiments, Ring B is of formula:

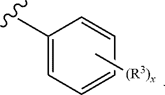

In certain embodiments, Ring B is of formula:

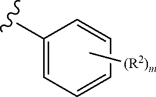

and m is 0 or 1. In certain embodiments, Ring B is of formula:

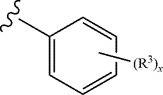

and m is 0, 1, 2, or 3. In certain embodiments, Ring B is of formula:

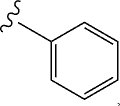

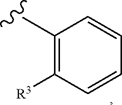

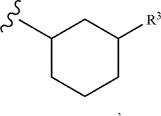

or

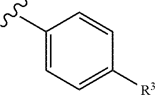

. In certain embodiments, Ring B is of formula:

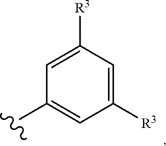

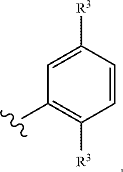

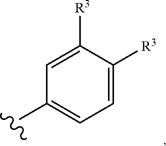

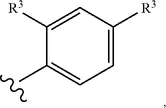

or

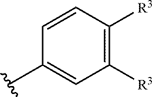

. In certain embodiments, Ring B is of formula:

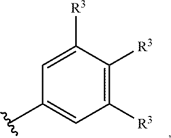

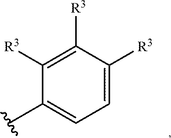

or

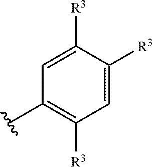

. In certain embodiments, Ring B is of formula:

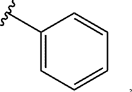

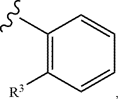

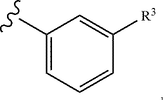

or

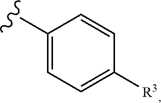

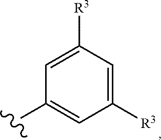

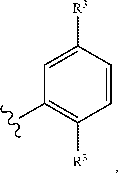

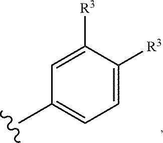

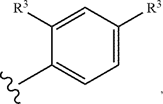

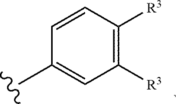

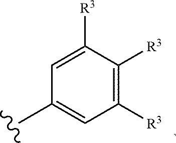

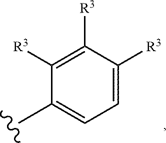

or

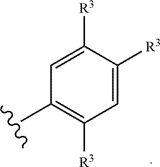

In certain embodiments, Ring B is of formula:

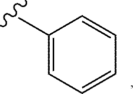

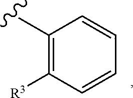

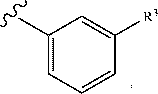

or

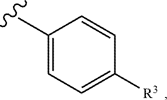

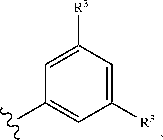

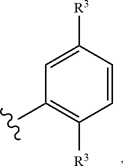

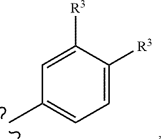

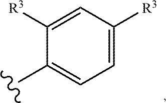

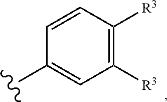

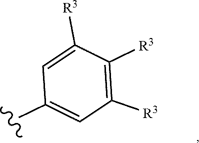

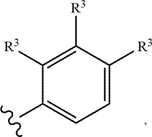

or

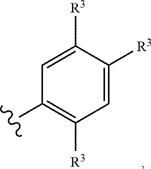

wherein R³ is halogen, optionally substituted acyl, optionally substituted alkyl, optionally substituted heteroalkyl, optionally substituted alkenyl, optionally substituted alkynyl, or optionally substituted carbocyclyl. In certain embodiments, Ring B is of formula:

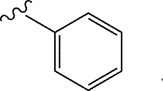

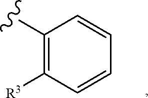

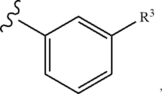

or

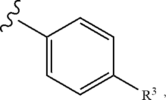

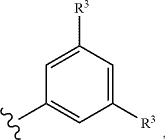

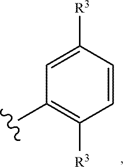

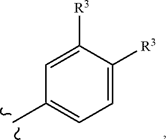

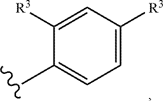

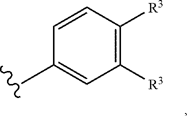

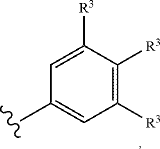

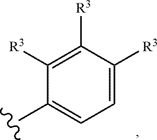

or

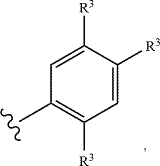

wherein R³ is halogen, optionally substituted acyl, optionally substituted alkyl, or optionally substituted carbocyclyl. In certain embodiments, Ring B is of formula:

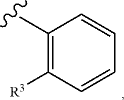

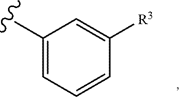

or

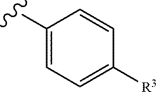

. In certain embodiments, Ring B is of formula:

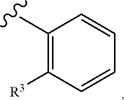

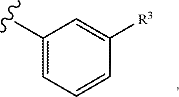

or

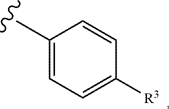

wherein R³ is halogen, optionally substituted acyl, alkyl optionally substituted with halogen, or optionally substituted C₃₋₁₄ carbocyclyl. In certain embodiments, the moiety

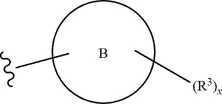

is of formula:

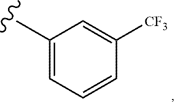

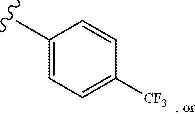

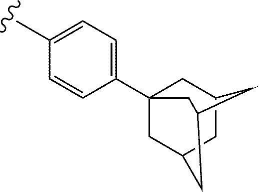

. In certain embodiments, Ring B is of formula:

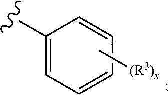

x is 0 or 1; and R³ is optionally substituted alkyl or optionally substituted carbocyclyl. In certain embodiments, Ring B is of formula:

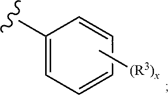

x is 1; and R³ is optionally substituted alkyl or optionally substituted carbocyclyl. In certain embodiments, Ring B is of formula:

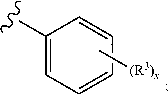

x is 0 or 1; and R³ is optionally substituted C₁₋₆ alkyl or optionally substituted C₃₋₁₄ carbocyclyl. In certain embodiments, Ring B is of formula:

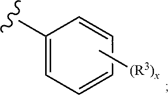

x is 0 or 1; and R³ is optionally substituted C₁₋₆ alkyl or optionally substituted adamantyl. In certain embodiments, Ring B is of formula:

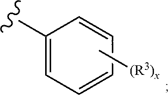

x is 1; and R³ is optionally substituted C₁₋₆ alkyl or optionally substituted C₃₋₁₄ carbocyclyl. In certain embodiments, Ring B is of formula:

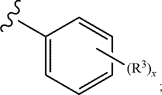

x is 0 or 1; and R³ is C₁₋₆ alkyl optionally substituted with halogen or optionally substituted C₃₋₁₄ carbocyclyl. In certain embodiments, Ring B is of formula:

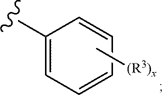

x is 1; and R³ is C₁₋₆ alkyl optionally substituted with halogen or optionally substituted C₃₋₁₄ carbocyclyl. In certain embodiments, Ring B is of formula:

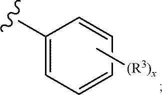

x is 0 or 1; and R³ is —CF₃ or adamantyl. In certain embodiments, Ring B is phenyl or cyclohexyl, x is 1; and R³ is —CF₃. In certain embodiments, Ring B is phenyl or cyclohexyl, x is 1; and R³ is adamantyl.

In certain embodiments, Ring B is cyclohexyl. In certain embodiments, Ring B is cyclohexyl substituted with one or more instances of substituent R³. In certain embodiments, Ring B is of formula:

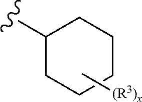

. In certain embodiments, Ring B is of formula:

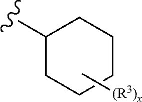

and x is 0, 1, or 2. In certain embodiments, Ring B is of formula:

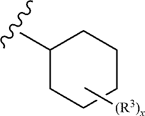

and x is 0, 1, 2, or 3. In certain embodiments, Ring B is of formula:

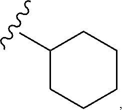

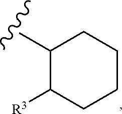

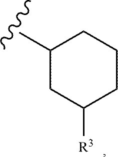

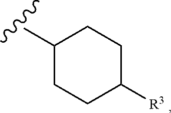

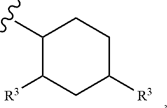

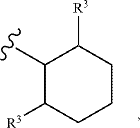

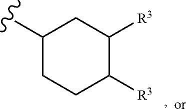

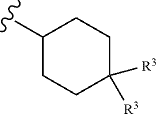

. In certain embodiments, Ring B is of formula:

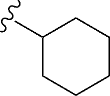

. In certain embodiments, Ring B is of formula:

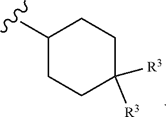

. In certain embodiments, Ring B is of formula:

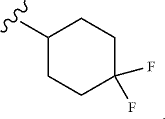

. In certain embodiments, the moiety

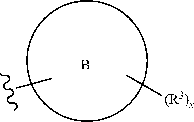

is of formula:

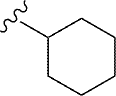

. In certain embodiments, Ring B is of formula:

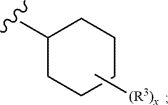

x is 0, 1, or 2; and R³ is halogen or optionally substituted alkyl. In certain embodiments, Ring B is of formula:

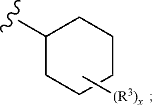

x is 1 or 2; and R³ is halogen or optionally substituted alkyl. In certain embodiments, Ring B is of formula:

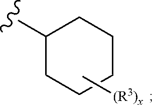

x is 1 or 2; and R³ is halogen or optionally substituted C₁₋₆ alkyl. In certain embodiments, Ring B is of formula:

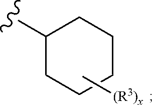

x is 2; and R³ is halogen. In certain embodiments, Ring B is of formula:

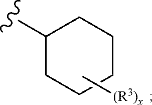

x is 1 or 2; and R³ is halogen or C₁₋₆ alkyl optionally substituted with halogen. In certain embodiments, Ring B is of formula:

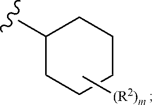

x is 2; and R³ is halogen or C₁₋₆ alkyl optionally substituted with halogen. In certain embodiments, Ring B is of formula:

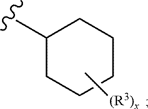

x is 1 or 2; and R³ is —F or —CF₃. In certain embodiments, Ring B is of formula:

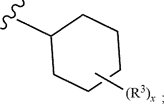

x is 1 or 2; and R³ is —F.

In certain embodiments, Ring B is of formula:

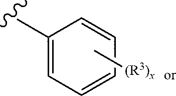

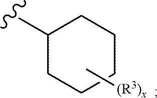

x is 0, 1, 2, or 3; and R³ is halogen, optionally substituted alkyl, optionally substituted alkenyl, optionally substituted alkynyl, or optionally substituted carbocyclyl. In certain embodiments, Ring B is of formula:

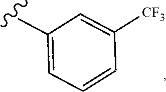

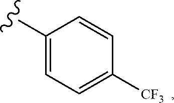

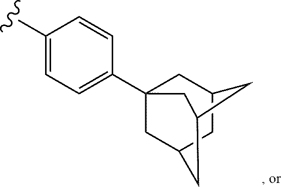

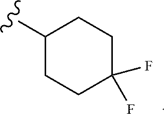

### Warhead of D¹

As generally defined herein, Formulas (I-A), (I-B), and (II) include substituent D¹, wherein D¹ is a warhead of any one of Formulae (i-1) to (i-23), (i-26) to (i-31), (i-34) to (i-40), (i-42), or (i-43):

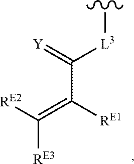

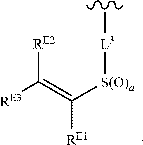

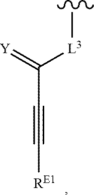

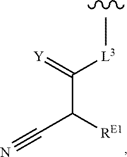

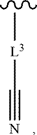

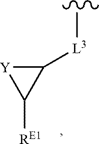

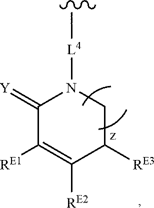

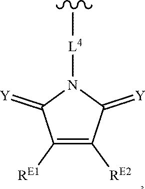

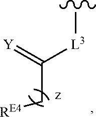

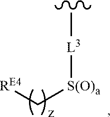

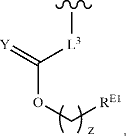

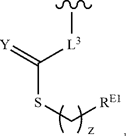

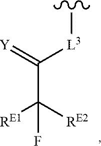

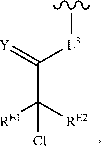

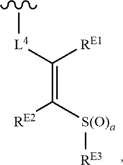

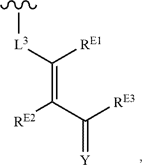

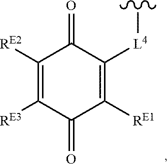

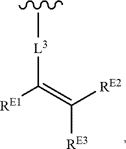

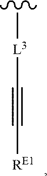

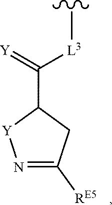

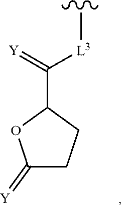

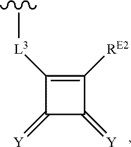

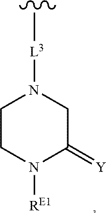

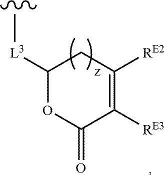

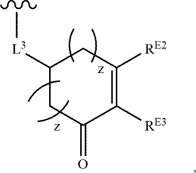

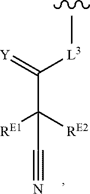

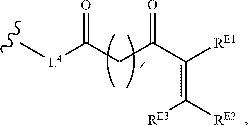

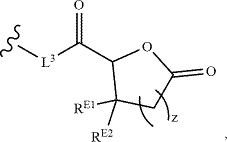

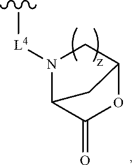

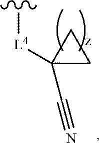

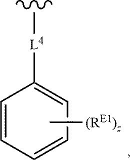

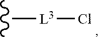

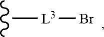

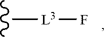

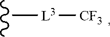

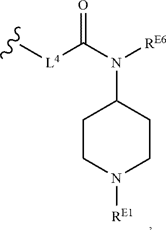

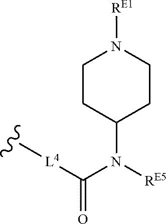

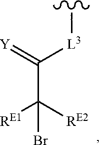

wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}–,—NR^{L3a}C(═O)—, —C(═O)NR^{L3a}–, —SC(═O)—, C(═O)S—, —OC(═O)—, —C(═O)O—, —NR^{L3a}C(═S)—, —C(═S)NR^{L3a}-, trans-CR^{L3b}═CR^{L3b}-, cis-CR^{L3b}═CR^{L3b}-, —C═C—, —S(═O)—, —S(═O)O—, —OS(═O)—, —S(═O)NR^{L3a}–, —NR^{L3a}S(═O)—, —S(═O)₂—, —S(═O)₂O—, —OS(═O)₂—, —S(═O)₂NR^{L3a}–, or —NR^{L3a}S(═O)₂-, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits.

In certain embodiments, D¹ is a warhead of any one of Formulae (i-1) to (i-23), (i-26) to (i-31), (i-34) to (i-40), (i-42), or (i-43). In certain embodiments, D¹ is a warhead of formula

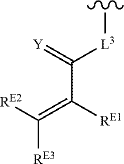

In certain embodiments, D¹ is of formula

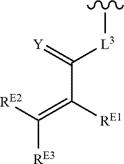

wherein L³ is a bond or optionally substituted C₁₋₄ alkyl and optionally wherein 1 to 2 carbon units of the C₁₋₄ alkyl are replaced with —C═O—, —NR^{L3a}–, or —NR^{L3a}C(═O)—; wherein R^{L3a} is hydrogen, substituted or unsubstituted C₁₋₆ alkyl, or a nitrogen protecting group; Y is O; and each of R^{E1}, R^{E2}, and R^{E3} is independently hydrogen, halogen, optionally substituted alkyl, optionally substituted alkenyl, or optionally substituted alkynyl. In certain embodiments, D¹ is of formula

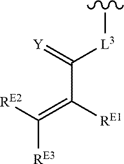

wherein L³ is a bond or optionally substituted C₁₋₄ alkyl and optionally wherein 1 to 2 carbon units of the C₁₋₄ alkyl are replaced with —C═O— or —NR^{L3a_}; wherein R^{L3a} is hydrogen, substituted or unsubstituted C₁₋₆ alkyl, or a nitrogen protecting group; Y is O; and each of R^{E1}, R^{E2}, and R^{E3} is independently hydrogen, halogen, optionally substituted alkyl, optionally substituted alkenyl, or optionally substituted alkynyl. In certain embodiments, D¹ is of formula

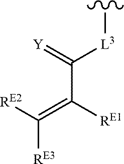

wherein L³ is a bond or optionally substituted C₁₋₄ alkyl and optionally wherein 1 to 2 carbon units of the C₁₋₄ alkyl are replaced with —C═O— or —NR^{L3a}–; wherein R^{L3a} is hydrogen, substituted or unsubstituted C₁₋₆ alkyl, or a nitrogen protecting group; Y is O; and each of R^{E1}, R^{E2}, and R^{E3} is independently hydrogen or optionally substituted alkyl. In certain embodiments, D¹ is of formula

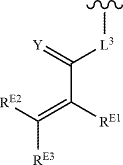

wherein L³ is a bond or optionally substituted C₁₋₄ alkyl and wherein 1 carbon unit of the C₁₋₄ alkyl are replaced with —C═O— or —NR^{L3a_}; R^{L3a} is hydrogen, substituted or unsubstituted C₁₋₆ alkyl, or a nitrogen protecting group; Y is O; and each of R^{E1}, R^{E2}, and R^{E3} is independently hydrogen or optionally substituted C₁₋₆ alkyl. In certain embodiments, D¹ is of formula

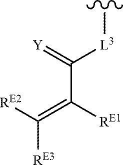

wherein L³ is a bond or optionally substituted C₁₋₄ alkyl and wherein 1 carbon unit of the C₁₋₄ alkyl are replaced with —C═O— or —NR^{L3a_}; R^{L3a} is hydrogen, substituted or unsubstituted C₁₋₆ alkyl, or a nitrogen protecting group; Y is O; and each of R^{E1}, R^{E2}, and R^{E3} is independently hydrogen or C₁₋₆ alkyl optionally substituted with —NH₂, —NH(optionally substituted alkyl), or -N(optionally substituted alkyl)₂. In certain embodiments, D¹ is a warhead of formula:

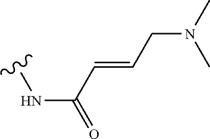

. In certain embodiments, D¹ is a warhead of formula:

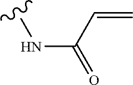

. In certain embodiments, D¹ is a warhead of formula:

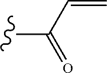

. In certain embodiments, D¹ is of formula:

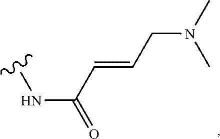

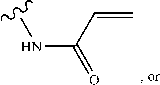

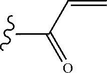

. In certain embodiments, D¹ is of formula:

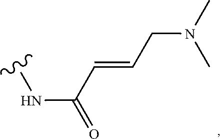

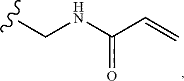

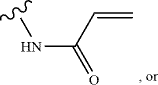

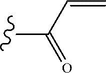

. In certain embodiments, D¹ is of formula:

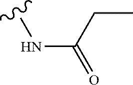

. In certain embodiments, D¹ is of formula:

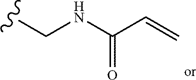

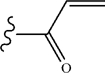

. In certain embodiments, D¹ is of formula

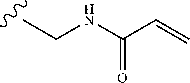

.

In certain embodiments, L3 is a bond. In certain embodiments, L3 is —NH. In certain embodiments, L³ is a bond. In certain embodiments, L³ is —NH—. In certain embodiments, R^{E1} and R^{E2} are hydrogen. In certain embodiments, R^{E1}, R^{E2}, and R^{E3} are all hydrogen. In certain embodiments, R^{E3} is —CH₂NMe₂. In certain embodiments, the warhead is of formula

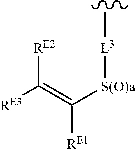

. In certain embodiments, the warhead is of formula

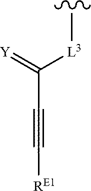

. In certain embodiments, the warhead is of formula

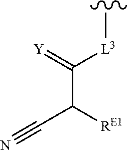

. In certain embodiments, the warhead is of formula

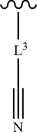

(i-5) . In certain embodiments, the warhead is of formula

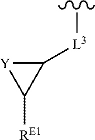

). In certain embodiments, the warhead is of formula

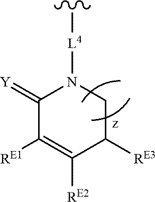

. In certain embodiments, the warhead is of formula

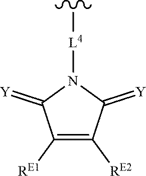

. In certain embodiments, the warhead is of formula

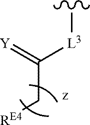

. In certain embodiments, the warhead D¹ is of formula

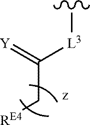

wherein L³ is a bond or optionally substituted C₁₋₄ alkyl and optionally wherein 1 to 2 carbon units of the C₁₋₄ alkyl are replaced with —C═O— or —NR^{L3a}–; wherein R^{L3a} is hydrogen, substituted or unsubstituted C₁₋₆ alkyl, or a nitrogen protecting group; Y is O; and R^{E4} is a leaving group (e.g., optionally substituted C₁₋₆ alkyl); and each instance of z is independently 0, 1, 2, or 3, as valency permits. In certain embodiments, the warhead D¹ is of formula

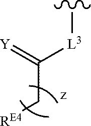

wherein L³ is a bond or optionally substituted C₁₋₄ alkyl and optionally wherein 1 to 2 carbon units of the C₁₋₄ alkyl are replaced with —C═O— or —NR^{L3a}-; wherein R^{L3a} is hydrogen, substituted or unsubstituted C₁₋₆ alkyl, or a nitrogen protecting group; Y is O; and R^{E4} is a leaving group (e.g., unsubstituted substituted C₁₋₆ alkyl); and each instance of z is independently 0, 1, 2, or 3, as valency permits. In certain embodiments, the warhead D¹ is of formula

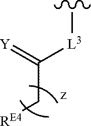

wherein L³ is unsubstituted C₁₋₄ alkyl and optionally wherein 1 carbon unit of the C₁₋₄ alkyl is replaced with —NR^{L3a}—; wherein R^{L3a} is hydrogen or substituted or unsubstituted C₁₋₆ alkyl; Y is O; and R^{E4} is unsubstituted substituted C₁₋₆ alkyl; and each instance of z is independently 0, 1, 2, or 3, as valency permits. In certain embodiments, D¹ is of formula:

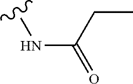

. In certain embodiments, the warhead is of formula

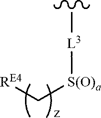

In certain embodiments, the warhead is

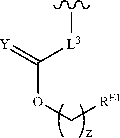

In certain embodiments, the warhead is of formula

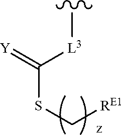

In certain embodiments, the warhead is of formula

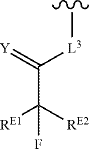

In certain embodiments, the warhead is of formula

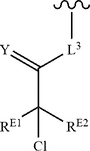

In certain embodiments, the warhead is of formula

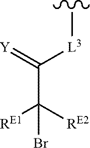

. In certain embodiments, the warhead is of formula

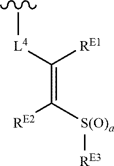

In certain embodiments, the warhead is of formula

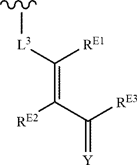

In certain embodiments, the warhead is of formula

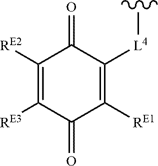

In certain embodiments, the warhead is of formula

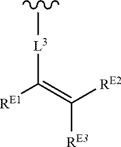

In certain embodiments, the warhead is of formula

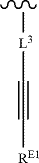

In certain embodiments, the warhead is of formula

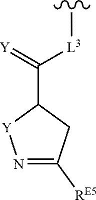

In certain embodiments, the warhead is

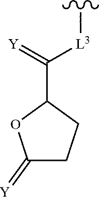

In certain embodiments, the warhead is of formula

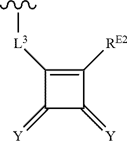

In certain embodiments, the warhead is of formula

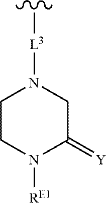

In certain embodiments, the warhead is of formula

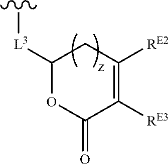

In certain embodiments, the warhead is of formula

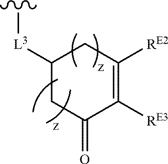

In certain embodiments, the warhead is of formula

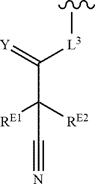

In certain embodiments, the warhead is of formula

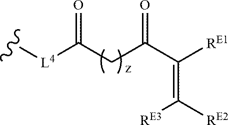

In certain embodiments, the warhead is of formula

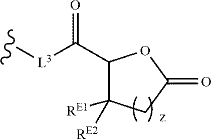

(i-30). In certain embodiments, the warhead is of formula

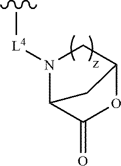

In certain embodiments, the warhead is of formula

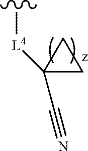

In certain embodiments, the warhead is of formula

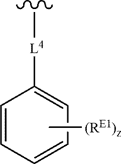

In certain embodiments, the warhead is of formula

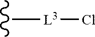

. In certain embodiments, the warhead is of formula

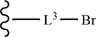

In certain embodiments, D¹ is a warhead is of formula

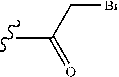

. In certain embodiments, the warhead is of formula

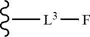

In certain embodiments, the warhead is of formula

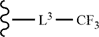

In certain embodiments, the warhead is of formula

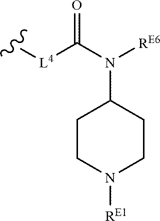

In certain embodiments, the warhead is of formula

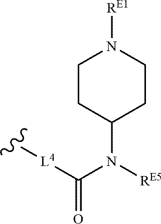

In certain embodiments, R^{3′} is a warhead of formula

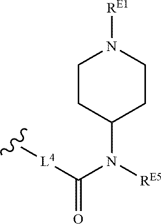

. In certain embodiments, R^{3′} is a warhead of formula

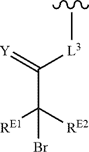

In certain embodiments, R³ is a warhead of formula (i-1) to (i-23), (i-26) to (i-31), (i-34) to (i-40), (i-42), or (i-43). In certain embodiments, the warhead is of formula

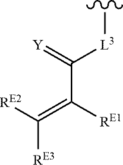

(i-1). In certain embodiments, R³ is a warhead of formula

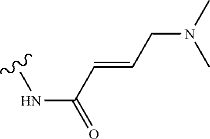

. In certain embodiments, R³ is a warhead of formula

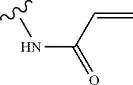

. In certain embodiments, R³ is a warhead of formula

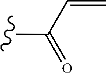

. In certain embodiments, R³ is of formula:

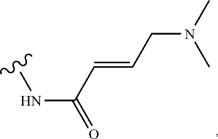

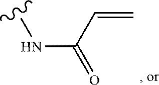

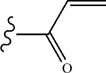

. In certain embodiments, R³ is of formula:

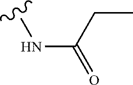

. In certain embodiments, L³ is a bond. In certain embodiments, L³ is —NH—. In certain embodiments, R^{E1} and R^{E2} are hydrogen. In certain embodiments, R^{E1}, R^{E2}, and R^{E3} are all hydrogen. In certain embodiments, R^{E3} is —CH₂NMe₂.

In certain embodiments, the warhead is of formula

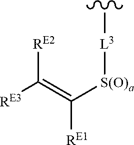

In certain embodiments, the warhead is of formula

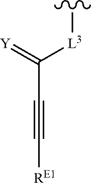

In certain embodiments, the warhead is of formula

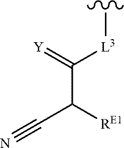

In certain embodiments, the warhead is of formula

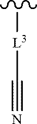

In certain embodiments, the warhead is of formula

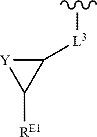

In certain embodiments, the warhead is of formula

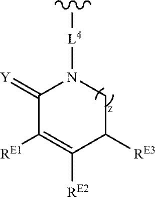

In certain embodiments, the warhead is of formula

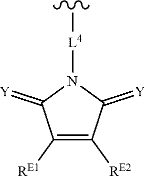

In certain embodiments, the warhead is of formula

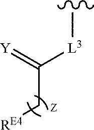

In certain embodiments, the warhead is of formula

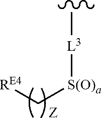

In certain embodiments, the warhead is

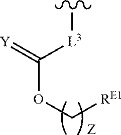

In certain embodiments, the warhead is of formula

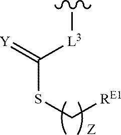

In certain embodiments, the warhead is of formula

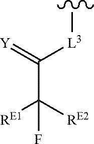

. In certain embodiments, the warhead is of formula

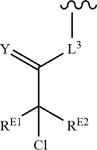

In certain embodiments, D¹ is a warhead is of formula

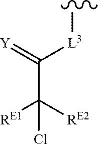

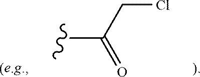

In certain embodiments, D¹ is a warhead is of formula

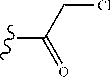

. In certain embodiments, the warhead is of formula

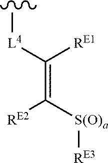

In certain embodiments, the warhead is of formula

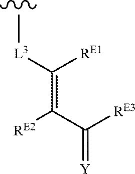

In certain embodiments, the warhead is of formula

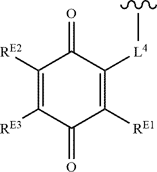

In certain embodiments, the warhead is of formula

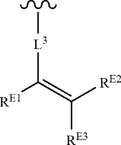

In certain embodiments, the warhead is of formula

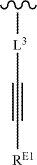

In certain embodiments, the warhead is of formula

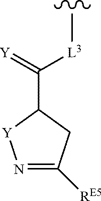

In certain embodiments, the warhead is

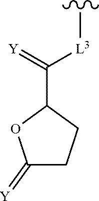

In certain embodiments, the warhead is of formula

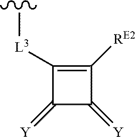

In certain embodiments, the warhead is of formula

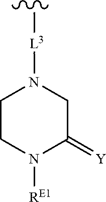

. In certain embodiments, the warhead is of formula

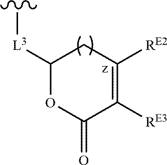

In certain embodiments, the warhead is of formula

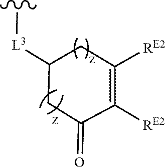

In certain embodiments, the warhead is of formula

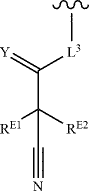

In certain embodiments, the warhead is of formula

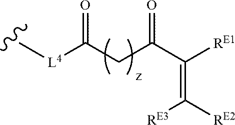

In certain embodiments, the warhead is of formula

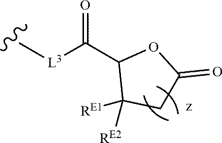

In certain embodiments, the warhead is of formula

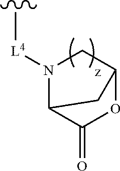

In certain embodiments, the warhead is of formula

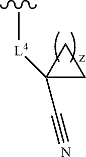

In certain embodiments, the warhead is of formula

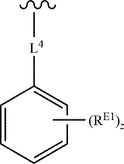

In certain embodiments, the warhead is of formula

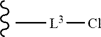

In certain embodiments, D¹ is a warhead is of formula

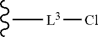

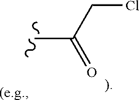

In certain embodiments, D¹ is a warhead is of formula

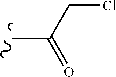

. In certain embodiments, the warhead is of formula

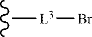

In certain embodiments, D¹ is a warhead is of formula

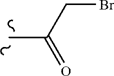

. In certain embodiments, the warhead is of formula

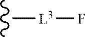

In certain embodiments, the warhead is of formula

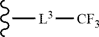

In certain embodiments, the warhead is of formula

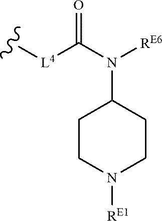

In certain embodiments, the warhead is of formula

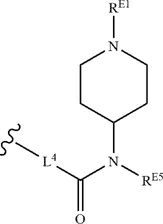

In certain embodiments, the warhead is of formula

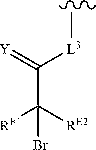

In certain embodiments, L³ is a bond (e.g., a single bond, a double bond, or a triple bond). In certain embodiments, L³ is a single bond. In certain embodiments, L³ is a double bond. In certain embodiments, L³ is a triple bond. In certain embodiments, L³ is an optionally substituted C ₁₋₄ hydrocarbon chain, optionally wherein one or more carbon units of the hydrocarbon chain are independently replaced with —C═O—, —O—, —S—, -NR^{L3a_}, —NR^{L3a}c(═0)—, —C(═O)NR^{L3a}—, SC(═O)—, —C(═O)S—, —OC(═O)—, —C(═O)O—, —NR ^{L3a}C(═S)—, —C(═S)NR^{L3a}—, trans-CR^{L3b}=CR^{L3b}-, cis-CR^{L3b}=CR^{L3b}-, —C≡C—, —S(═O)—, —S(═O)O—, —OS(═O)—, —S(═O)NR^{L3a}—, —NR^{L3a}S(═O)—, —S(═O)₂—, —S(═O)₂O—, —OS(═O)2—, —S(═O)₂NR^{L3a}–, or —NR^{L3a}S(═O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring. In certain embodiments, L⁴ is a bond (e.g., a single bond, a double bond, or a triple bond). In certain embodiments, L⁴ is an optionally substituted branched C₁₋₆ hydrocarbon chain (e.g., i-Pr). In certain embodiments, L⁴ is an optionally substituted unbranched C₁₋₆ hydrocarbon chain (e.g., n-Pr, or n-Bu). In certain embodiments, at least one instance of R^{E1} is H. In certain embodiments, at least one instance of R^{E1} is halogen (e.g., F, Cl, Br, or I). In certain embodiments, at least one instance of R^{E1} is optionally substituted alkyl (e.g., Me, or Et). In certain embodiments, at least one instance of R^{E1} is optionally substituted alkenyl (e.g., optionally substituted vinyl). In certain embodiments, at least one instance of R^{E1} is optionally substituted alkynyl. In certain embodiments, at least one instance of R^{E1} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{E1} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, at least one instance of R^{E1} is substituted or unsubstituted aryl (e.g., substituted or unsubstituted, 6- to 10-membered aryl). In certain embodiments, at least one instance of R^{E1} is substituted or unsubstituted phenyl. In certain embodiments, at least one instance of R^{E1} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, at least one instance of R^{E1} is —CN. In certain embodiments, at least one instance of R^{E1} is —CH₂OR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl. In certain embodiments, at least one instance of R^{E1} is —CH₂N(R^{EF})₂ or —N(R^{EF}), wherein each instance of R^{EF} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, optionally wherein two R^{EF} groups are joined to form an optionally substituted heterocyclic ring. In certain embodiments, at least one instance of R^{E1} is -CH₂SR^{EE} or -SR^{EE} (e.g., —CH₂SMe or -SMe). In certain embodiments, at least one instance of R^{E1} is -OR^{EE} (e.g., -OMe). In certain embodiments, at least one instance of R^{E1} is —Si(R^{EG}), wherein each instance of R^{EG} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl (e.g., —Si(Me)₃).

In certain embodiments, at least one instance of R^{E2} is H. In certain embodiments, at least one instance of R^{E2} is halogen (e.g., F, Cl, Br, or I). In certain embodiments, at least one instance of R^{E2} is optionally substituted alkyl (e.g., Me, or Et). In certain embodiments, at least one instance of R^{E2} is optionally substituted alkenyl (e.g., optionally substituted vinyl). In certain embodiments, at least one instance of R^{E2} is optionally substituted alkynyl. In certain embodiments, at least one instance of R^{E2} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{E2} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, at least one instance of R^{E2} is substituted or unsubstituted aryl (e.g., substituted or unsubstituted, 6- to 10-membered aryl). In certain embodiments, at least one instance of R^{E2} is substituted or unsubstituted phenyl. In certain embodiments, at least one instance of R^{E2} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, at least one instance of R^{E2} is —CN. In certain embodiments, at least one instance of R^{E2} is -CH₂OR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl. In certain embodiments, at least one instance of R^{E2} is —CH₂N(R^{EF})₂ or N(R^{EF})₂, wherein each instance of R^{EF} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, optionally wherein two R^{EF} groups are joined to form an optionally substituted heterocyclic ring. In certain embodiments, at least one instance of R^{E2} is —CH₂SR^{EE} or -SR^{EE} (e.g., —CH₂SMe or -SMe). In certain embodiments, at least one instance of R^{E2} is -OR^{EE} (e.g., -OMe). In certain embodiments, at least one instance of R^{E2} is —Si(R^{EG})₃, wherein each instance of R^{EG} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl (e.g., —Si(Me)₃). In certain embodiments, at least one instance of R^{E3} is H. In certain embodiments, at least one instance of R^{E3} is halogen (e.g., F, Cl, Br, or I). In certain embodiments, at least one instance of R^{E3} is optionally substituted alkyl (e.g., Me, or Et). In certain embodiments, at least one instance of R^{E3} is optionally substituted alkenyl (e.g., optionally substituted vinyl). In certain embodiments, at least one instance of R^{E3} is optionally substituted alkynyl. In certain embodiments, at least one instance of R^{E3} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{E3} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, at least one instance of R^{E3} is substituted or unsubstituted aryl (e.g., substituted or unsubstituted, 6- to 10-membered aryl). In certain embodiments, at least one instance of R^{E3} is substituted or unsubstituted phenyl. In certain embodiments, at least one instance of R^{E3} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, at least one instance of R^{E3} is —. In certain embodiments, at least one instance of R^{E3} is —CH₂OR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl. In certain embodiments, at least one instance of R^{E3} is —CH₂N(R^{EF})₂ or —N(R^{EF})₂, wherein each instance of R^{EF} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, optionally wherein two R^{EF} groups are joined to form an optionally substituted heterocyclic ring. In certain embodiments, at least one instance of R^{E3} is —CH₂SR^{EE} or —SR^{EE} (e.g., —CH₂SMe or —SMe). In certain embodiments, at least one instance of R^{E3} is -OR^{EE} (e.g., -OMe). In certain embodiments, at least one instance of R^{E3} is —Si(R^{EG}), wherein each instance of R^{EG} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl (e.g., —Si(Me)₃). In certain embodiments, R^{E1} and R^{E3} are joined to form an optionally substituted carbocyclic ring (e.g., substituted or unsubstituted, 3- to 7-membered, monocyclic carbocyclyl comprising zero, one, or two double bonds in the carbocyclic ring system). In certain embodiments, R^{E1} and R^{E3} are joined to form an optionally substituted heterocyclic ring (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{E2} and R^{E3} are joined to form an optionally substituted carbocyclic ring (e.g., substituted or unsubstituted, 3- to 7-membered, monocyclic carbocyclyl comprising zero, one, or two double bonds in the carbocyclic ring system). In certain embodiments, R^{E2} and R^{E3} are joined to form an optionally substituted heterocyclic ring (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{E1} and R^{E2} are joined to form an optionally substituted carbocyclic ring (e.g., substituted or unsubstituted, 3- to 7-membered, monocyclic carbocyclyl comprising zero, one, or two double bonds in the carbocyclic ring system). In certain embodiments, R^{E1} and R^{E2} are joined to form an optionally substituted heterocyclic ring (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{E4} is a leaving group (e.g., halogen, or a sulfonic acid ester, e.g., -O(tosylate) or -O(mesylate)). In certain embodiments, R^{E5} is halogen (e.g., F, Cl, Br, or I). In certain embodiments, R^{E6} is H. In certain embodiments, R^{E6} is substituted or unsubstituted C₁₋₆ alkyl (e.g., Me, is —CF₃, Bn, Et, perfluoroethyl, Pr, perfluoropropyl, Bu, or perfluorobutyl). In certain embodiments, R^{E6} is a nitrogen protecting group (e.g., Bn, Boc, Cbz, Fmoc, trifluoroacetyl, triphenylmethyl, acetyl, or Ts). In certain embodiments, at least one instance of Y is O. In certain embodiments, at least one instance of Y is S. In certain embodiments, at least one instance of Y is NR^{E7}, wherein R^{E7} is hydrogen, substituted or unsubstituted C₁₋₆ alkyl, or a nitrogen protecting group (e.g., NMe). In certain embodiments, a is 1. In certain embodiments, a is 2. In certain embodiments, at least one instance of z is 0. In certain embodiments, at least one instance of z is 1. In certain embodiments, at least one instance of z is 2. In certain embodiments, at least one instance of z is 3. In certain embodiments, at least one instance of z is 4. In certain embodiments, at least one instance of z is 5. In certain embodiments, at least one instance of z is 6.

In certain embodiments, D¹ is a warhead of formula (i-1) to (i-19), (i-22), (i-23), (i-27) to (i-29), (i-34) to (i-29), or (i-43). In certain embodiments, D¹ is a warhead of formula

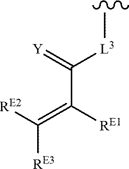

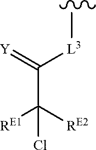

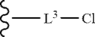

or

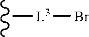

In certain embodiments, D¹ is a warhead of formula

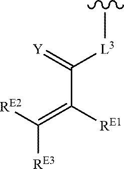

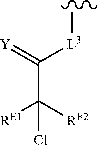

or

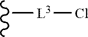

. In certain embodiments, D¹ is a warhead of formula,

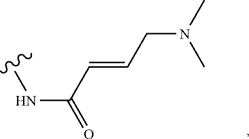

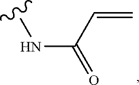

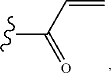

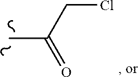

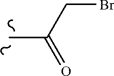

. In certain embodiments, D¹ is a warhead of formula,

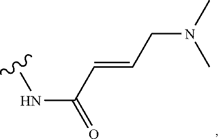

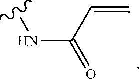

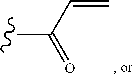

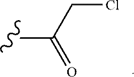

### Linker X¹

Formula (I-A) includes linker X¹ attaching Ring B to the moiety of formula:

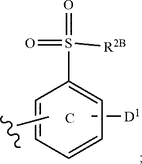

and Formula (I-B) includes linker X¹ attaching Ring B to the moiety of formula:

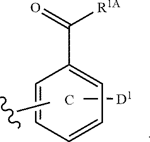

. Formula (II) includes linker X¹ attaching Ring B to the moiety of formula:

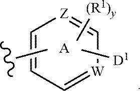

In certain embodiments, in compounds of Formulae (I-A), (I-B), and (II), X¹ is —O—, -O(alkylene)-, alkylene, —S—, —SCH₂—, —N(R^{da})—, or —N(R^{da})CH₂—, wherein R^{da} is as defined herein. In certain embodiments, X¹ is —O—, —O(CH₂)_{1—10}—, —(CH₂)_{1—10}—, —S—, —SCH₂—, —N(R^{da})—, or —N(R^{da})CH₂—, wherein R^{da} is as defined herein. In certain embodiments, X¹ is —O—, —O(CH₂)_{1—6}—, —(CH₂)_{1—6}—, —S—, or —N(R^{da})—, wherein R^{da} is as defined herein.

In certain embodiments, in compounds of Formulae (I-A), (I-B), or (II), X¹ is —O—.

In certain embodiments, in of Formulae (I-A), (I-B), or (II), X¹ is -O(alkylene)-. In certain embodiments, X¹ is —O(CR^{d})_{1—6}, and R^{d} is hydrogen, halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN. In certain embodiments, at least one instance of R^{d} is hydrogen. In certain embodiments, at least one instance of R^{d} is halogen (e.g., F, Cl, Br, or I). In certain embodiments, at least one instance of R^{d} is optionally substituted acyl (e.g., —C(═O)Me). In certain embodiments, at least one instance of R^{d} is optionally substituted alkyl (e.g., substituted or unsubstituted C₁₋₆ alkyl). In certain embodiments, at least one instance of R^{d} is alkyl optionally substituted with halogen. In certain embodiments, at least one instance of R² is optionally substituted C₁₋₆ alkyl. In certain embodiments, at least one instance of R^{d} is substituted or unsubstituted methyl. In certain embodiments, at least one instance of R^{d} is substituted or unsubstituted ethyl. In certain embodiments, at least one instance of R^{d} is substituted or unsubstituted propyl. In certain embodiments, at least one instance of R^{d} is optionally substituted alkenyl (e.g., substituted or unsubstituted C₂₋₆ alkenyl). In certain embodiments, at least one instance of R^{d} is optionally substituted alkynyl (e.g., substituted or unsubstituted C₂₋₆ alkynyl). In certain embodiments, at least one instance of R^{d} is optionally substituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{d} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{d} is optionally substituted aryl (e.g., substituted or unsubstituted, 6- to 10-membered aryl). In certain embodiments, at least one instance of R^{d} is benzyl. In certain embodiments, at least one instance of R^{d} is substituted or unsubstituted phenyl. In certain embodiments, at least one instance of R^{d} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{d} is —OR^{c1} (e.g., —OH or —OMe). In certain embodiments, at least one instance of R^{d} is —N(R^{c2})₂ (e.g., —NMe₂). In certain embodiments, at least one instance of R^{d} is —SR^{c1} (e.g., -SMe). In certain embodiments, at least one instance of R^{d} is —NO₂. In certain embodiments, at least one instance of R^{d} is —CN. In certain embodiments, at least one instance of R^{d} is —SCN.

In certain embodiments, in compounds of Formulas (I-A) or (I-B), X¹ is of formula:

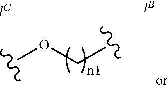

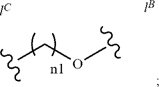

l^{C} indicates the point of attachment to the moiety of formula:

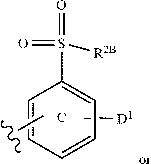

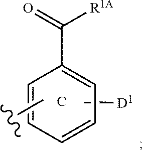

and l^{B} indicates the point of attachment to Ring B; and n1 is 1, 2, 3, 4, 5, or 6. In certain embodiments, X¹ is of formula:

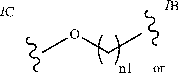

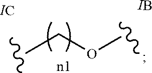

l^{C} indicates the point of attachment to the moiety of formula:

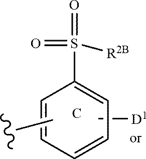

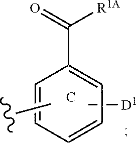

and l^{B} indicates the point of attachment to Ring B; and n1 is 1, 2, 3, or 4. In certain embodiments, X¹ is of formula:

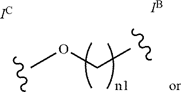

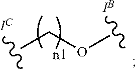

l^{C} indicates the point of attachment to the moiety of formula:

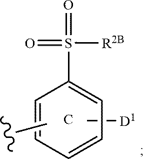

and l^{B} indicates the point of attachment to Ring B; and n1 is 1, 2, 3, 4, 5, or 6. In certain embodiments, X¹ is of formula:

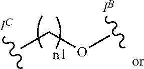

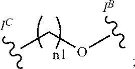

l^{C} indicates the point of attachment to the moiety of formula:

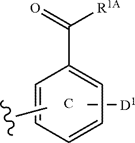

; and l^{B} indicates the point of attachment to Ring B; and n1 is 1, 2, 3, 4, 5, or 6. In certain embodiments, n1 is 1. In certain embodiments, n1 is 2. In certain embodiments, n1 is 3. In certain embodiments, n1 is 4. In certain embodiments, n1 is 5. In certain embodiments, n1 is 6. In certain embodiments, n1 is 1, 2, 3, or 4. In certain embodiments, n1 is 1, 2, or 3. In certain embodiments, X¹ is of formula:

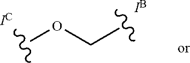

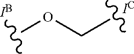

. In certain embodiments, X¹ is of formula:

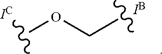

In certain embodiments, in compounds of Formula (II), X¹ is of formula:

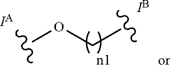

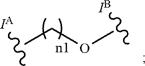

l^{A} indicates the point of attachment to the moiety of formula:

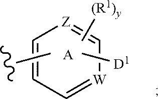

and l^{B} indicates the point of attachment to Ring B; and n1 is 1, 2, 3, 4, 5, or 6. In certain embodiments, n1 is 1. In certain embodiments, n1 is 2. In certain embodiments, n1 is 3. In certain embodiments, n1 is 4. In certain embodiments, n1 is 5. In certain embodiments, n1 is 6. In certain embodiments, n1 is 1, 2, 3, or 4. In certain embodiments, n1 is 1, 2, or 3. In certain embodiments, X¹ is of formula:

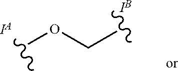

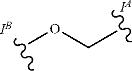

. In certain embodiments, X¹ is of formula:

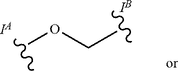

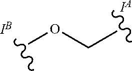

. In certain embodiments, X¹ is of formula:

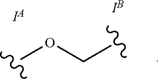

In certain embodiments, in compounds of Formulae (I-A), (I-B), or (II), X¹ is alkylene. In certain embodiments, X¹ is -(CR^{d})ₙ₁-, wherein R^{d} and n1 are as defined herein. In certain embodiments, X¹ is —(CR^{d})ₙ₁—, wherein R^{d} is hydrogen, halogen, optionally substituted acyl, optionally substituted alkyl, optionally substituted alkenyl, optionally substituted alkynyl, optionally substituted carbocyclyl, -OR^{c1}, —NO₂, —N(R^{c2})₂, —SR^{c1}, —CN, or —SCN; and n1 is 1, 2, 3, 4, 5, 6, 7, 8, 9, or 10. In certain embodiments, X¹ is —(CR^{d})ₙ₁—, wherein R^{d} is hydrogen, halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1},—CN, or —SCN; and n1 is 1, 2, 3, 4, 5, or 6. In certain embodiments, n1 is 1. In certain embodiments, n1 is 2. In certain embodiments, n1 is 3. In certain embodiments, n1 is 4. In certain embodiments, n1 is 5. In certain embodiments, n1 is 6. In certain embodiments, n1 is 7. In certain embodiments, n1 is 8. In certain embodiments, n1 is 9. In certain embodiments, n1 is 10. In certain embodiments, n1 is 1, 2, 3, 4, 5, 6, 7, or 8. In certain embodiments, n1 is 1, 2, 3, 4, or 5. In certain embodiments, n1 is 1, 2, 3, or 4. In certain embodiments, n1 is 1, 2, or 3. In certain embodiments, X¹ is —CH₂—, —CH₂CH₂—, or —CH₂CH₂CH₂—. In certain embodiments, X¹ is —CH₂—. In certain embodiments, X¹ is —CH₂— or —CH₂CH₂—. In certain embodiments, X¹ is —CH₂CH₂—. In certain embodiments, X¹ is —CH₂CH₂CH₂—.

In certain embodiments, in compounds of Formulae (I-A), (I-B), or (II), X¹ is —S—.

In certain embodiments, in compounds of Formulae (I-A), (I-B), or (II), X¹ is —SCH₂—.

In certain embodiments, in compounds of Formulae (I-A), (I-B), or (II), X¹ is —N(R^{da})—, wherein R^{da} is as defined herein. In certain embodiments, R^{da} is hydrogen, optionally substituted alkyl, optionally substituted acyl, or a nitrogen protecting group. In certain embodiments, R^{da} is hydrogen. In certain embodiments, R^{da} is optionally substituted acyl (e.g., —C(═O)Me). In certain embodiments, R^{da} is optionally substituted alkyl (e.g., substituted or unsubstituted C₁₋₆ alkyl). In certain embodiments, R^{da} is optionally substituted methyl, optionally substituted ethyl, optionally substituted propyl, or optionally substituted butyl. In certain embodiments, R^{da} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, X¹ is —N(R^{da})—, and R^{da} is hydrogen, optionally substituted alkyl (e.g., optionally substituted C₁₋₆ alkyl), or a nitrogen protecting group. In certain embodiments, X¹ is —N(R^{da})—, and R^{da} is hydrogen or optionally substituted C₁₋₆ alkyl. In certain embodiments, X¹ is —NH—.

In certain embodiments, in compounds of Formulae (I-A), (I-B), or (II), X¹ is —N(R^{da})CH₂—, wherein R^{da} is as defined herein (e.g., —NHCH₂—). In certain embodiments, X¹ is —NHCH₂—.

### Substituent R^{2B}

Formula (I-A) includes substituent R^{2B} as part of the moiety

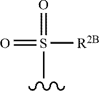

attached to Ring C. In certain embodiments, R^{2B} is —N(R^{c2})₂, —OR^{c1}, optionally substituted alkyl, optionally substituted alkenyl, optionally substituted alkynyl, optionally substituted carbocyclyl, optionally substituted heterocyclyl, optionally substituted aryl, or optionally substituted heteroaryl, wherein R^{c1} and R^{c2} are as defined herein. In certain embodiments, R^{2B} is —N(R^{c2})₂ (e.g., —NH₂). In certain embodiments, R^{2B} is —N(R^{c2})₂, and each instance of R^{c2} is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or a nitrogen protecting group. In certain embodiments, R^{2B} is —N(R^{c2})₂ (e.g., —NH₂). In certain embodiments, R^{2B} is —N(R^{c2})₂, and each instance of R^{c2} is independently hydrogen, optionally substituted alkyl, optionally substituted alkenyl, optionally substituted alkynyl, or optionally substituted carbocyclyl. In certain embodiments, R^{2B} is —N(R^{c2})₂, and each instance of R^{c2} is independently hydrogen, optionally substituted alkyl, or optionally substituted carbocyclyl. In certain embodiments, R^{2B} is —N(R^{c2})₂, and at least one instance of R^{c2} is hydrogen. In certain embodiments, R^{2B} is —NH(R^{c2}), wherein R^{c2} is optionally substituted alkyl or optionally substituted carbocyclyl. In certain embodiments, R^{2B} is —NH(R^{c2}), wherein R^{c2} is optionally substituted C₁₋₆ alkyl or optionally substituted C₃₋₁₀ carbocyclyl. In certain embodiments, R^{2B} is —NHMe or

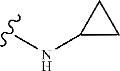

. In certain embodiments, R^{2B} is —NHMe. In certain embodiments, R^{2B} is

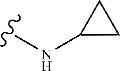

. In certain embodiments, R^{2B} is —NMe₂. In certain embodiments, R^{2B} is -OR^{c1} (e.g., —OH or —OMe). In certain embodiments, R^{2B} is optionally substituted alkyl (e.g., substituted or unsubstituted C₁₋₆ alkyl). In certain embodiments, at least one instance of R^{2B} is alkyl optionally substituted with halogen. In certain embodiments, at least one instance of R^{2B} is optionally substituted C₁₋₆ alkyl. In certain embodiments, at least one instance of R^{2B} is C₁₋₆ alkyl optionally substituted with halogen. In certain embodiments, at least one instance of R^{2B} is optionally substituted methyl. In certain embodiments, at least one instance of R^{2B} is substituted or unsubstituted ethyl. In certain embodiments, at least one instance of R^{2B} is substituted or unsubstituted propyl. In certain embodiments, at least one instance of R^{2B} is optionally substituted alkenyl (e.g., substituted or unsubstituted C₂₋₆ alkenyl). In certain embodiments, at least one instance of R^{2B} is optionally substituted alkynyl (e.g., substituted or unsubstituted C₂₋₆ alkynyl). In certain embodiments, at least one instance of R^{2B} is optionally substituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{2B} is optionally substituted heterocyclyl (e.g., substituted or unsubstituted, 5-to 10-membered monocyclic or bicyclic heterocyclic ring, wherein one or two atoms in the heterocyclic ring are independently nitrogen, oxygen, or sulfur). In certain embodiments, at least one instance of R^{2B} is optionally substituted aryl (e.g., substituted or unsubstituted, 6- to 10-membered aryl). In certain embodiments, at least one instance of R^{2B} is benzyl. In certain embodiments, at least one instance of R^{2B} is substituted or unsubstituted phenyl. In certain embodiments, at least one instance of R^{2B} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

In certain embodiments, in a compound of Formula (I-A), the moiety of formula:

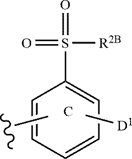

is of formula:

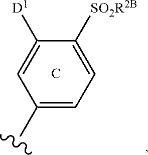

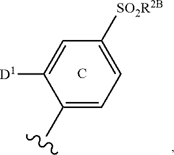

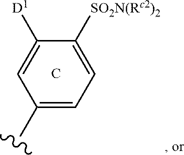

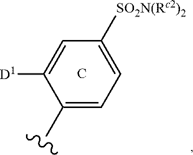

wherein R^{2B} and R^{c2} are as described herein. In certain embodiments, the moiety of formula:

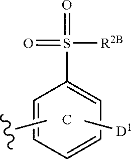

is of formula:

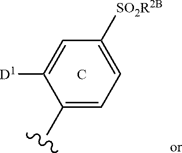

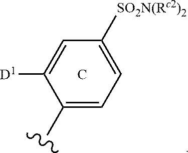

In certain embodiments, the moiety of formula:

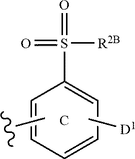

is of formula:

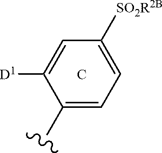

or

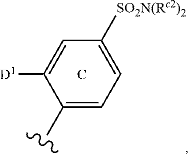

wherein R^{2B} is optionally substituted alkyl and each instance of R^{c2} is hydrogen, optionally substituted alkyl, or optionally substituted carbocyclyl. In certain embodiments, the moiety of formula:

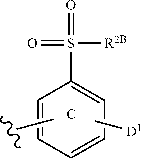

is of formula:

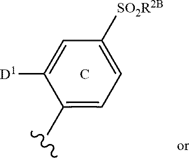

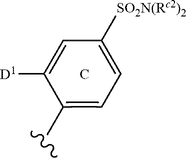

. In certain embodiments, the moiety of formula:

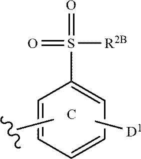

is of formula:

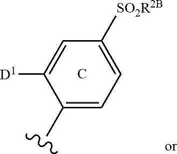

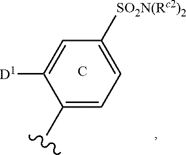

wherein R^{2B} is optionally substituted alkyl and each instance of R^{c2} is hydrogen, optionally substituted alkyl, or optionally substituted carbocyclyl; and D¹ is a warhead of formula

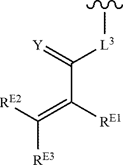

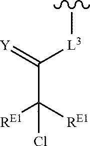

or

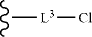

. In certain embodiments, the moiety of formula:

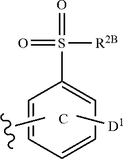

is of formula:

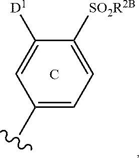

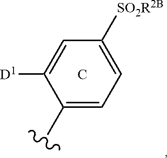

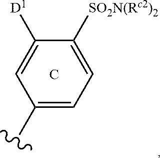

or

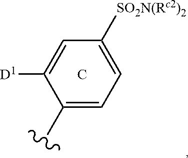

wherein R^{2B} is optionally substituted alkyl and each instance of R^{c2} is hydrogen, optionally substituted alkyl, or optionally substituted carbocyclyl; and D¹ is a warhead of formula

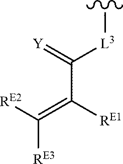

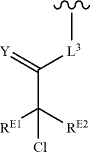

or

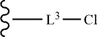

In certain embodiments, the moiety of formula:

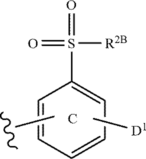

is of formula:

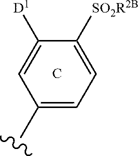

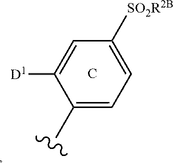

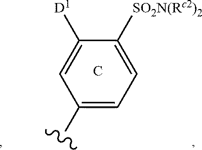

or

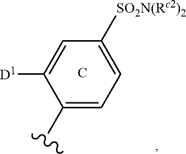

wherein R^{2B} is optionally substituted alkyl and each instance of R^{c2} is hydrogen, optionally substituted alkyl, or optionally substituted carbocyclyl; and D¹ is a warhead of formula

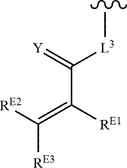

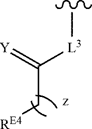

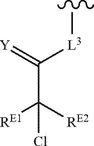

or

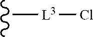

In certain embodiments, the moiety of formula:

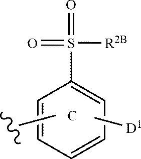

is of formula:

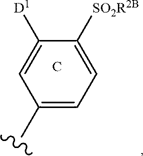

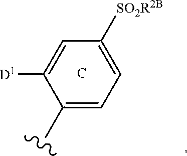

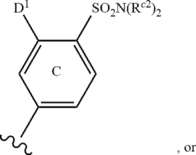

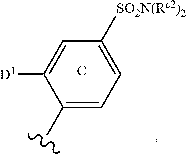

wherein R^{2B} is optionally substituted alkyl and each instance of R^{c}² is hydrogen, optionally substituted C₁₋₆ alkyl, or optionally substituted carbocyclyl; and D¹ is a warhead of formula

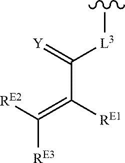

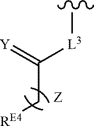

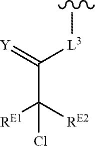

or

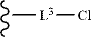

In certain embodiments, the moiety of formula:

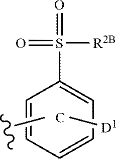

is of formula:

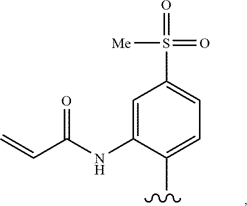

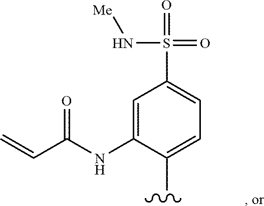

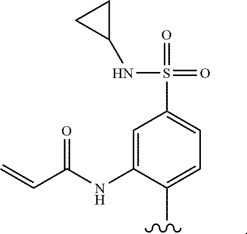

In certain embodiments, the moiety of formula:

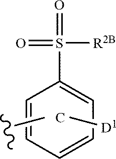

is of formula:

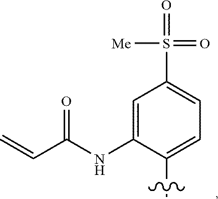

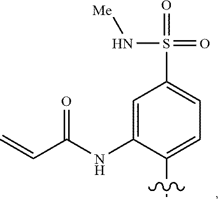

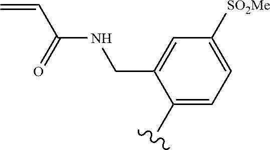

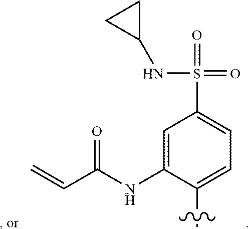

### Substituent R^{A1}

Formula (I-B) includes substituent R^{A1} as part of the moiety

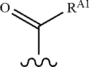

attached to Ring C. In certain embodiments, R^{A1} is —O(R^{a2}) or —N(R^{a3})₂, wherein R^{a2} and R^{a3} are as defined herein. In certain embodiments, R^{A1} is —O(R^{a2}) (e.g., —OH or —OMe). In certain embodiments, R^{A1} is —OH. In certain embodiments, R^{A1} is not —OH. In certain embodiments, R^{A1} is —OR^{a2}, and R^{a2} is hydrogen or optionally substituted alkyl (e.g., optionally substituted C₁₋₆ alkyl, such as optionally substituted methyl, optionally substituted ethyl, optionally substituted propyl, optionally substituted butyl). In certain embodiments, R^{A1} is —OMe. In certain embodiments, R^{A1} is —OEt. In certain embodiments, R^{A1} is -O(n-propyl). In certain embodiments, R^{A1} is -O(isopropyl). In certain embodiments, R^{A1} is -O(butyl). In certain embodiments, R^{A1} is -O(n-butyl). In certain embodiments, R^{A1} is -O(t-butyl). In certain embodiments, R^{A1} is -O(i-butyl). In certain embodiments, R^{A1} is -O(s-butyl). In certain embodiments, R^{a2} is hydrogen. In certain embodiments, R^{a2} is optionally substituted acyl (e.g., —C(═O)Me). In certain embodiments, R^{a2} is optionally substituted alkyl (e.g., substituted or unsubstituted C₁₋₆ alkyl). In certain embodiments, R^{a2} is substituted or unsubstituted methyl. In certain embodiments, R^{a2} is substituted or unsubstituted ethyl. In certain embodiments, R^{a2} is substituted or unsubstituted propyl. In certain embodiments, R^{a2} is optionally substituted alkenyl (e.g., substituted or unsubstituted C₂₋₆ alkenyl). In certain embodiments, R^{a2} is optionally substituted alkynyl (e.g., substituted or unsubstituted C₂₋₆ alkynyl). In certain embodiments, R^{a2} is optionally substituted carbocyclyl (e.g., substituted or unsubstituted, 3- to 10-membered, monocyclic carbocyclyl comprising zero, one, or two double bonds in the carbocyclic ring system). In certain embodiments, R^{a2} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{a2} is optionally substituted aryl (e.g., substituted or unsubstituted, 6- to 10-membered aryl). In certain embodiments, R^{a2} is benzyl. In certain embodiments, R^{a2} is substituted or unsubstituted phenyl. In certain embodiments, R^{a2} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{a2} is an oxygen protecting group.

In certain embodiments, R^{A1} is —N(R^{a3})₂ (e.g., -NH₂). In certain embodiments, R^{A1} is —N(R^{a3})₂, wherein at least one instance of R^{a3} is hydrogen, optionally substituted alkyl, optionally substituted carbocyclyl, or —SO₂(R^{a4}), and R^{a4} is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R^{A1} is —N(R^{a3})₂, wherein at least one instance of R^{a3} is hydrogen, optionally substituted C₁₋₆ alkyl, optionally substituted C₃₋₁₀ carbocyclyl, or —SO₂(R^{a4}), and R^{a4} is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R^{A1} is —N(R^{a3})₂, wherein at least one instance of R^{a3} is hydrogen, optionally substituted C₁₋₆ alkyl, optionally substituted C₃₋₁₀ carbocyclyl, or —SO₂(R^{a4}), and R^{a4} is optionally substituted alkyl, optionally substituted alkenyl, or optionally substituted alkynyl. In certain embodiments, R^{A1} is —NH(R^{a3}). In certain embodiments, R^{A1} is —NH(R^{a3}), and R^{a3} is as defined herein. In certain embodiments, R^{A1} is —NH(R^{a3}), wherein R^{a3} is optionally substituted C₁₋₆ alkyl, optionally substituted C₃₋₁₀ carbocyclyl, or —SO₂(R^{a4}). In certain embodiments, R^{A1} is

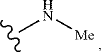

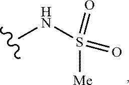

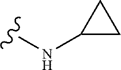

, or —NMe₂. In certain embodiments, R^{A1} is -NH(optionally substituted alkyl, -NH(optionally substituted carbocyclyl), or -NH(SO₂(optionally substituted alkyl)). In certain embodiments, R^{A1} is -NH(optionally substituted C₁₋₆ alkyl, -NH(optionally substituted C₃₋₁₀ carbocyclyl), or -NH(SO₂(optionally substituted C₁₋₆ alkyl)).

In certain embodiments, at least one instance of R^{a3} is hydrogen. In certain embodiments, at least one instance of R^{a3} is optionally substituted acyl (e.g., —C(═O)Me. In certain embodiments, at least one instance of R^{a3} is optionally substituted alkyl (e.g., substituted or unsubstituted C₁₋₆ alkyl). In certain embodiments, at least one instance of R^{a3} is optionally substituted C₁₋₆ alkyl (e.g., optionally substituted methyl, optionally substituted ethyl, optionally substituted propyl, optionally substituted butyl). In certain embodiments, at least one instance of R^{a3} is substituted or unsubstituted methyl. In certain embodiments, at least one instance of R^{a3} is unsubstituted methyl. In certain embodiments, both instances of R^{a3} are substituted or unsubstituted methyl. In certain embodiments, both instances of R^{a3} are unsubstituted methyl. In certain embodiments, at least one instance of R^{a3} is substituted methyl. In certain embodiments, at least one instance of R^{a3} is substituted or unsubstituted ethyl. In certain embodiments, at least one instance of R^{a3} is substituted or unsubstituted propyl (e.g., n-propyl, isopropyl). In certain embodiments, at least one instance of R^{a3} is substituted or unsubstituted butyl (e.g., n-butyl, s-butyl, isobutyl, t-butyl). In certain embodiments, at least one instance of R^{a3} is optionally substituted alkenyl (e.g., substituted or unsubstituted C₂₋₆ alkenyl). In certain embodiments, at least one instance of R^{a3} is optionally substituted alkynyl (e.g., substituted or unsubstituted C₂₋₆ alkynyl). In certain embodiments, at least one instance of R^{a3} is optionally substituted carbocyclyl (e.g., substituted or unsubstituted, 3- to 10-membered, monocyclic carbocyclyl comprising zero, one, or two double bonds in the carbocyclic ring system). In certain embodiments, at least one instance of R^{a3} is optionally substituted C₃₋₁₄ carbocyclyl. In certain embodiments, at least one instance of R^{a3} is optionally substituted C₃₋₁₀ carbocyclyl. In certain embodiments, at least one instance of R^{a3} is C₃₋₁₀ carbocyclyl optionally substituted with halogen, -OR^{c1} (e.g., —OH, -O(alkyl), —CN, —SCN, —NO₂, or —N(R^{c2})₂ (e.g., —NH₂, or —NMe₂). In certain embodiments, at least one instance of R^{a3} is optionally substituted cyclopropyl, optionally substituted cyclobutyl, optionally substituted pentyl, optionally substituted cyclohexyl, or optionally substituted cycloheptyl. In certain embodiments, at least one instance of R^{a3} is optionally substituted cyclopropyl. In certain embodiments, at least one instance of R^{a3} is unsubstituted cyclopropyl. In certain embodiments, at least one instance of R^{a3} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{a3} is optionally substituted aryl (e.g., substituted or unsubstituted, 6- to 10-membered aryl). In certain embodiments, at least one instance of R^{a3} is benzyl. In certain embodiments, at least one instance of R^{a3} is substituted or unsubstituted phenyl. In certain embodiments, at least one instance of R^{a3} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9-to 10-membered, bicyclic heteroaryl, wherein one, two, three, or four atoms in the heteroaryl ring system are independently nitrogen, oxygen, or sulfur). In certain embodiments, at least one instance of R^{a3} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{a3} are taken together with their intervening atoms to form a substituted or unsubstituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or substituted or unsubstituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{a3} is —SO₂(R^{a4}) (e.g., —SO₂Me, —SO₂Et,

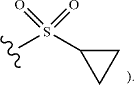

In certain embodiments, at least one instance of R^{a3} is —SO₂(R^{a4}) (e.g., —SO₂Me, —SO₂Et, -SO₂(cyclopropyl)). In certain embodiments, at least one instance of R^{a3} is —SO₂(R^{a4}), and R^{a4} is optionally substituted alkyl, optionally substituted alkenyl, optionally substituted alkynyl, or optionally substituted carbocyclyl. In certain embodiments, at least one instance of R^{a3} is —SO₂(R^{a4}), and R^{a4}is optionally substituted alkyl, optionally substituted alkenyl, or optionally substituted alkynyl. In certain embodiments, at least one instance of R^{a3} is —SO₂Me. In certain embodiments, at least one instance of R^{a3} is SO₂Et. In certain embodiments, at least one instance of R^{a3} is —SO₂Me, —SO₂Et, or

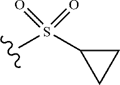

. In certain embodiments, at least one instance of R^{a3} is hydrogen and the other instance of R^{a3} is optionally substituted C₁₋₆ alkyl, optionally substituted C₃₋₁₀ carbocyclyl, or —SO₂(R^{a4}). In certain embodiments, at least one instance of Ra³ is hydrogen and the other instance of R^{a3} is optionally substituted methyl, optionally substituted cyclopropyl, or —SO₂Me. In certain embodiments, both instances of R^{a}³ are optionally substituted C₁₋₆ alkyl. In certain embodiments, both instances of R^{a3} are optionally substituted methyl. In certain embodiments, both instances of R^{a3} are methyl.

In certain embodiments, at least one instance of R ^{a3} is —SO₂(R^{a4}), and R^{a4} is as defined herein. In certain embodiments, R^{a4} is optionally substituted alkyl (e.g., substituted or unsubstituted C₁₋₆ alkyl). In certain embodiments, R^{a4} is substituted or unsubstituted methyl. In certain embodiments, R^{a4} is substituted or unsubstituted ethyl. In certain embodiments, R^{a4} is substituted or unsubstituted propyl. In certain embodiments, R^{a4} is optionally substituted alkenyl (e.g., substituted or unsubstituted C₂₋₆ alkenyl). In certain embodiments, R^{a4} is optionally substituted alkynyl (e.g., substituted or unsubstituted C₂₋₆ alkynyl). In certain embodiments, R^{a4} is optionally substituted carbocyclyl (e.g., substituted or unsubstituted, 3-to 10-membered, monocyclic carbocyclyl comprising zero, one, or two double bonds in the carbocyclic ring system). In certain embodiments, R^{a4} is optionally substituted C₃₋₇ carbocyclyl. In certain embodiments, R^{a4} is optionally substituted cyclopropyl. In certain embodiments, R^{a4} is optionally substituted cyclobutyl. In certain embodiments, R^{a4} is optionally substituted cyclopentyl. In certain embodiments, R^{a4} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{a4} is optionally substituted aryl (e.g., substituted or unsubstituted, 6- to 10-membered aryl). In certain embodiments, R^{a4} is benzyl. In certain embodiments, R^{a4} is substituted or unsubstituted phenyl. In certain embodiments, R^{a4} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{a4} is optionally substituted alkyl, optionally substituted alkenyl, optionally substituted alkynyl, or optionally substituted carbocyclyl. In certain embodiments, at least one instance of R^{a4} is optionally substituted alkyl, optionally substituted alkenyl, or optionally substituted alkynyl.

In certain embodiments, as part of the moiety

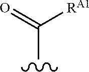

attached to Ring C, R^{A1} is

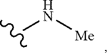

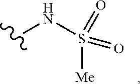

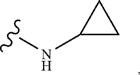

-NMe₂, or —OH.

In certain embodiments, in a compound of Formula (I-B), the moiety of formula:

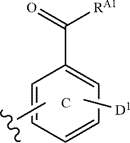

is of formula:

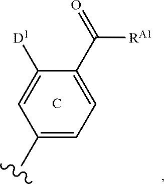

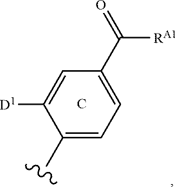

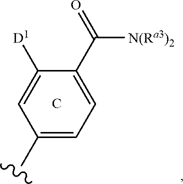

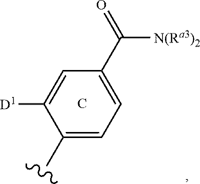

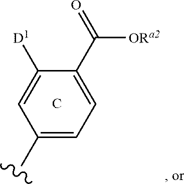

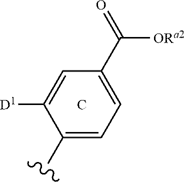

. In certain embodiments, the moiety of formula:

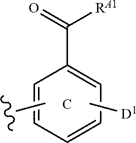

is of formula:

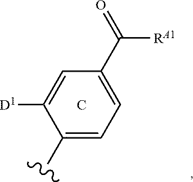

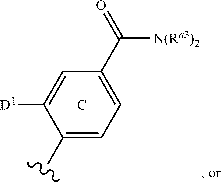

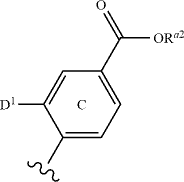

. In certain embodiments, the moiety of formula:

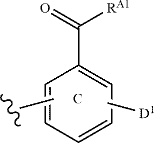

is of formula:

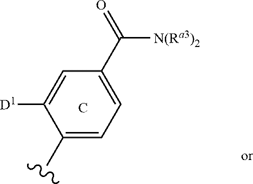

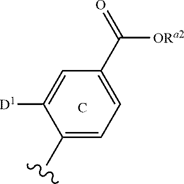

, wherein R^{a2} is hydrogen or optionally substituted alkyl; and each instance of R^{a3} is optionally hydrogen, optionally substituted alkyl, optionally substituted carbocyclyl, or -SO₂(optionally substituted alkyl). In certain embodiments, in a compound of Formula (I-B), the moiety of formula:

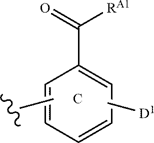

is of formula:

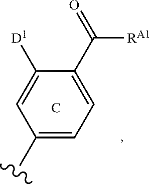

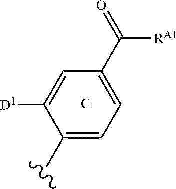

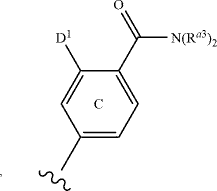

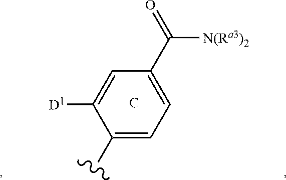

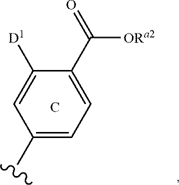

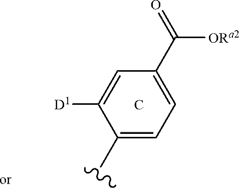

, wherein R^{a2} is hydrogen or optionally substituted alkyl; and each instance of R^{a3} is optionally hydrogen, optionally substituted alkyl, optionally substituted carbocyclyl, or -SO₂(optionally substituted alkyl); and D¹ is a warhead of formula

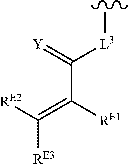

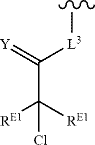

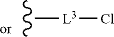

In certain embodiments, the moiety of formula:

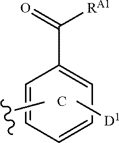

is of formula:

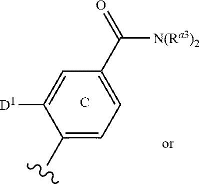

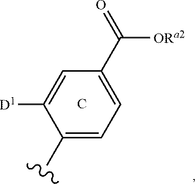

wherein R^{a2} is hydrogen or optionally substituted alkyl; and each instance of R^{a3} is optionally hydrogen, optionally substituted alkyl, optionally substituted carbocyclyl, or -SO₂(optionally substituted alkyl); and D¹ is a warhead of formula:

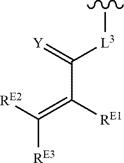

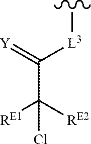

or

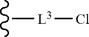

In certain embodiments, the moiety of formula:

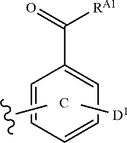

is of formula:

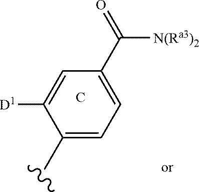

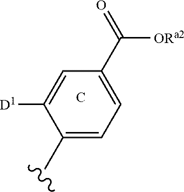

, wherein R^{a2} is hydrogen or optionally substituted alkyl; and each instance of R^{a3} is optionally hydrogen, optionally substituted alkyl, optionally substituted carbocyclyl, or -SO₂(optionally substituted alkyl); and D¹ is a warhead of formula:

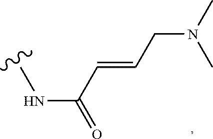

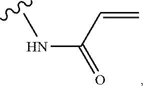

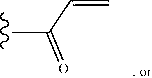

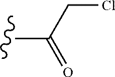

. In certain embodiments, the moiety of formula:

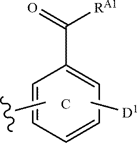

is of formula:

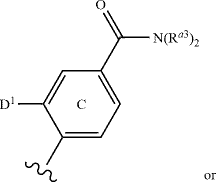

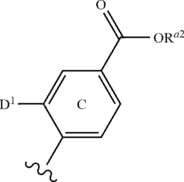

, wherein R^{a2} is hydrogen or optionally substituted alkyl; each instance of R^{a3} is independently hydrogen, optionally substituted alkyl, optionally substituted carbocyclyl, or -SO₂(optionally substituted alkyl); and D¹ is a warhead of formula:

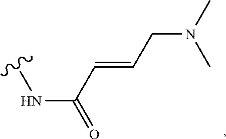

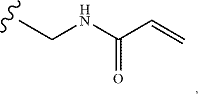

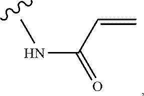

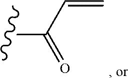

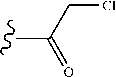

. In certain embodiments, the moiety of formula:

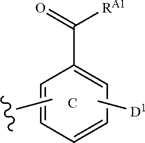

is of formula:

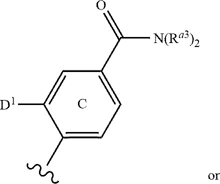

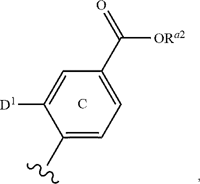

wherein R^{a2} is hydrogen or optionally substituted alkyl; and each instance of R^{a3} is optionally hydrogen, optionally substituted alkyl, optionally substituted carbocyclyl, or -SO₂(optionally substituted alkyl); and D¹ is a warhead of formula:

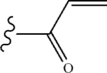

. In certain embodiments, the moiety of formula:

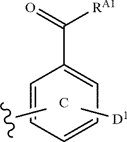

is of formula:

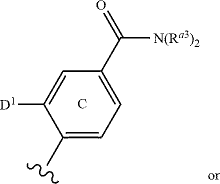

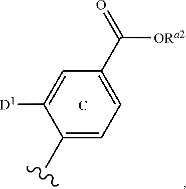

wherein R^{a2} is hydrogen or optionally substituted alkyl; and each instance of R^{a3} is independently hydrogen, optionally substituted alkyl, optionally substituted carbocyclyl, or -SO₂(optionally substituted alkyl); and D¹ is a warhead of formula:

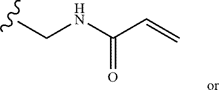

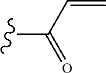

. In certain embodiments, the moiety of formula:

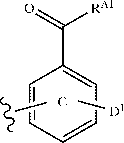

is of formula:

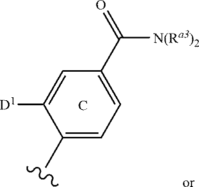

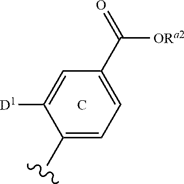

, wherein R^{a2} is hydrogen or optionally substituted alkyl; and each instance of R^{a3} is optionally hydrogen, optionally substituted alkyl, optionally substituted carbocyclyl, or -SO₂(optionally substituted alkyl); and D¹ is a warhead of formula:

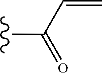

. In certain embodiments, the moiety of formula:

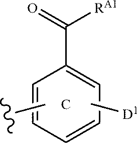

is of formula:

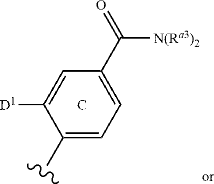

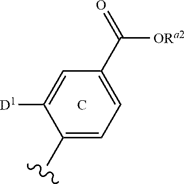

, wherein R^{a2} is hydrogen or optionally substituted alkyl; and each instance of R^{a3} is optionally hydrogen, optionally substituted alkyl, optionally substituted carbocyclyl, or -SO₂(optionally substituted alkyl); and D¹ is a warhead of formula:

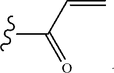

In certain embodiments, the moiety of formula:

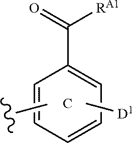

is of formula:

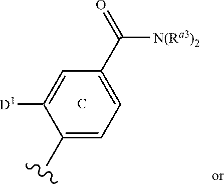

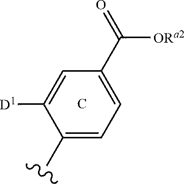

, wherein R^{a2} is hydrogen or optionally substituted alkyl; each instance of R^{a3} is optionally hydrogen, optionally substituted alkyl, optionally substituted carbocyclyl, or -SO₂(optionally substituted alkyl); and D¹ is a warhead of formula:

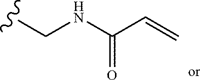

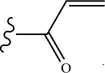

In certain embodiments, the moiety of formula:

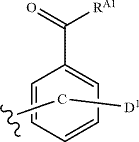

is of formula:

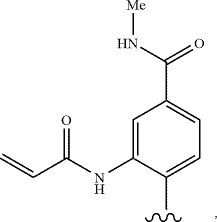

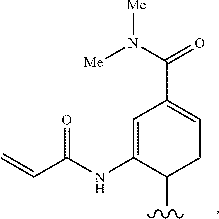

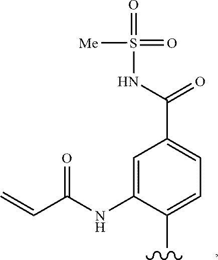

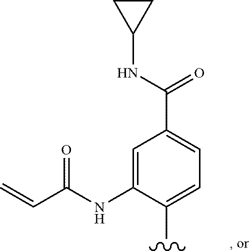

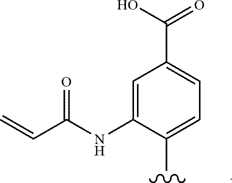

. In certain embodiments, the moiety of formula:

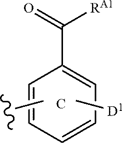

is of formula:

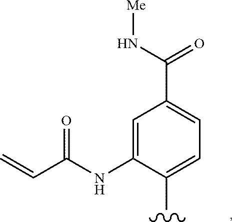

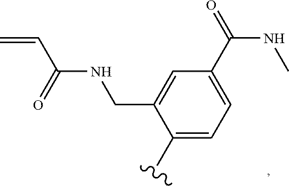

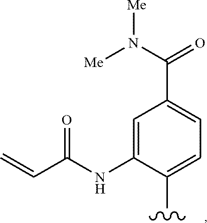

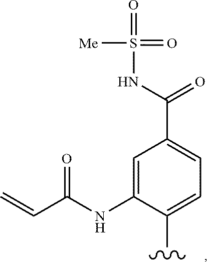

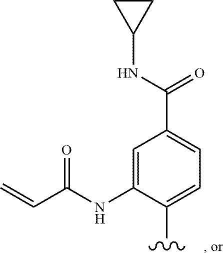

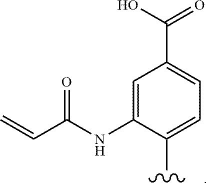

### Ring A of Formula (II)

As generally defined herein, in Formula (II), in the moiety

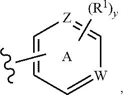

Ring A includes W and Z, which are as defined herein, provided at least one instance of W and Z is —C(R^{a})═ or —C(R^{b})═. In certain embodiments, W is —C(R^{a})═ or —N═, as valency permits; and R^{a} is hydrogen, halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN. In certain embodiments, R^{a} is hydrogen. In certain embodiments, R^{a} is halogen (e.g., F, Cl, Br, or I). In certain embodiments, R^{a} is optionally substituted acyl (e.g., —C(═O)Me). In certain embodiments, R^{a} is optionally substituted alkyl (e.g., substituted or unsubstituted C₁₋₆ alkyl). In certain embodiments, R^{a} is alkyl optionally substituted with halogen. In certain embodiments, R^{a} is optionally substituted C₁₋₆ alkyl. In certain embodiments, R^{a} is substituted or unsubstituted methyl. In certain embodiments, R^{a} is substituted or unsubstituted ethyl. In certain embodiments, R^{a} is substituted or unsubstituted propyl. In certain embodiments, R^{a} is optionally substituted alkenyl (e.g., substituted or unsubstituted C₂₋₆ alkenyl). In certain embodiments, R^{a} is optionally substituted alkynyl (e.g., substituted or unsubstituted C₂₋₆ alkynyl). In certain embodiments, R^{a} is optionally substituted carbocyclyl (e.g., substituted or unsubstituted, 3- to 10-membered, monocyclic carbocyclyl comprising zero, one, or two double bonds in the carbocyclic ring system). In certain embodiments, R^{a} is —OR^{c1} (e.g., —OH or —OMe). In certain embodiments, R^{a} is —N(R^{c2})₂ (e.g., —NMe₂). In certain embodiments, R^{a} is —SR^{c1} (e.g., —SMe). In certain embodiments, R^{a} is —NO₂. In certain embodiments, R^{a} is —CN. In certain embodiments, R^{a} is —SCN. In certain embodiments, W is —CH═ or —N═. In certain embodiments, W is —CH. In certain embodiments, W is —N═.

In certain embodiments, in Formula (II), Z is —C(R^{b})═ or —N═, as valency permits; and R^{b} is hydrogen, halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN. In certain embodiments, R^{b} is hydrogen. In certain embodiments, R^{b} is halogen (e.g., F, Cl, Br, or I). In certain embodiments, R^{b} is optionally substituted acyl (e.g., —C(═O)Me). In certain embodiments, R^{b} is optionally substituted alkyl (e.g., substituted or unsubstituted C₁₋₆ alkyl). In certain embodiments, R^{b} is alkyl optionally substituted with halogen. In certain embodiments, R^{b} is optionally substituted C₁₋₆ alkyl. In certain embodiments, R^{b} is substituted or unsubstituted methyl. In certain embodiments, R^{b} is substituted or unsubstituted ethyl. In certain embodiments, R^{b} is substituted or unsubstituted propyl. In certain embodiments, R^{b} is optionally substituted alkenyl (e.g., substituted or unsubstituted C₂₋₆ alkenyl). In certain embodiments, R^{b} is optionally substituted alkynyl (e.g., substituted or unsubstituted C₂₋₆ alkynyl). In certain embodiments, R^{b} is optionally substituted carbocyclyl (e.g., substituted or unsubstituted, 3- to 10-membered, monocyclic carbocyclyl comprising zero, one, or two double bonds in the carbocyclic ring system). In certain embodiments, R^{b} is —OR^{c1} (e.g., —OH or —OMe). In certain embodiments, R^{b} is —N(R^{c2})₂ (e.g., —NMe₂). In certain embodiments, R^{b} is -SR^{c1} (e.g., —SMe). In certain embodiments, R^{b} is —NO₂. In certain embodiments, R^{b} is —CN. In certain embodiments, R^{b} is —SCN. In certain embodiments, Z is —CH═ or —N═. In certain embodiments, Z is —CH═. In certain embodiments, Z is —N═.

In certain embodiments, Z and W are both —CH═. In certain embodiments, Z is —N═ and W is —CH═. In certain embodiments, Z is —CH═ and W is —N═.

In certain embodiments, Ring A is of formula:

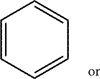

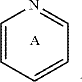

In certain embodiments, Ring A of the moiety

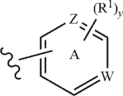

is substituted with zero or more R¹ substituents. In certain embodiments, y is 0. In certain embodiments, y is 1. In certain embodiments, y is 2. In certain embodiments, y is 3. In certain embodiments, y is 4. In certain embodiments, in the moiety

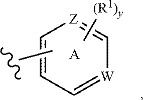

y is 0, 1, or 2. In certain embodiments, in the moiety

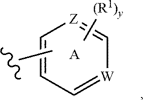

y is 0 or 1. In certain embodiments, at least one instance of R¹ is halogen (e.g., F, Cl, Br, or I). In certain embodiments, at least one instance of R¹ is —F. In certain embodiments, y is 1 and R¹ is halogen (e.g., F, Cl, Br, or I). In certain embodiments, at least one instance of R¹ is —Br. In certain embodiments, at least one instance of R¹ is —F. In certain embodiments, y is 1 and R¹ is —F. In certain embodiments, at least one instance of R¹ is —I. In certain embodiments, at least one instance of R¹ is optionally substituted acyl (e.g., —C(═O)Me). In certain embodiments, at least one instance of R¹ is optionally substituted alkyl (e.g., substituted or unsubstituted C₁₋₆ alkyl). In certain embodiments, at least one instance of R¹ is alkyl optionally substituted with halogen. In certain embodiments, at least one instance of R¹ is optionally substituted C₁₋₆ alkyl. In certain embodiments, at least one instance of R¹ is C₁₋₆ alkyl optionally substituted with halogen. In certain embodiments, at least one instance of R¹ is —CF₃. In certain embodiments, at least one instance of R¹ is substituted or unsubstituted methyl. In certain embodiments, at least one instance of R¹ is substituted or unsubstituted ethyl. In certain embodiments, at least one instance of R¹ is substituted or unsubstituted propyl. In certain embodiments, at least one instance of R¹ is optionally substituted alkenyl (e.g., substituted or unsubstituted C₂₋₆ alkenyl). In certain embodiments, at least one instance of R¹ is optionally substituted alkynyl (e.g., substituted or unsubstituted C₂₋₆ alkynyl). In certain embodiments, at least one instance of R¹ is optionally substituted carbocyclyl (e.g., substituted or unsubstituted, 3- to 14-membered, monocyclic carbocyclyl comprising zero, one, or two double bonds in the carbocyclic ring system). In certain embodiments, at least one instance of R¹ is —OR^{c1} (e.g., —OH or —OMe). In certain embodiments, at least one instance of R¹ is -N(R^{c2})₂ (e.g., —NMe₂). In certain embodiments, at least one instance of R¹ is —SR^{c1} (e.g., —SMe). In certain embodiments, at least one instance of R¹ is —NO₂. In certain embodiments, at least one instance of R¹ is —CN. In certain embodiments, at least one instance of R¹ is —SCN.

In certain embodiments, the moiety of formula:

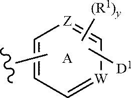

is of formula:

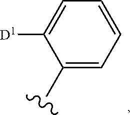

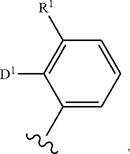

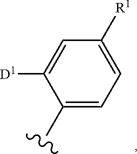

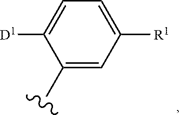

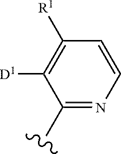

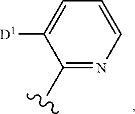

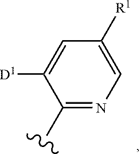

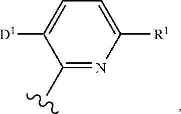

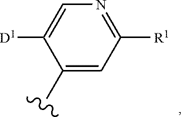

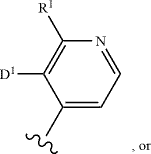

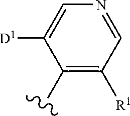

. In certain embodiments, the moiety of formula:

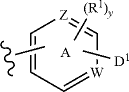

is of formula:

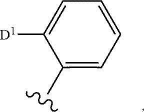

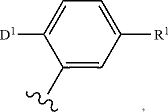

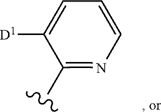

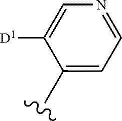

. In certain embodiments, the moiety of formula:

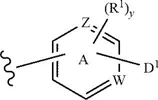

is of formula:

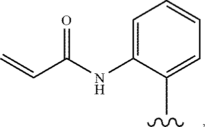

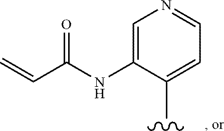

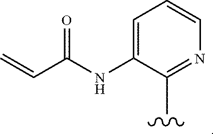

### Subgenera of Compounds of Formula (I-A)

In certain embodiments, the compound of Formula (I-A) is of formula:

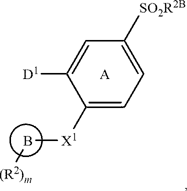

or a pharmaceutically acceptable salt, solvate, hydrate, polymorph, co-crystal, tautomer, stereoisomer, isotopically labeled derivative, or prodrug thereof.

In certain embodiments, the compound of Formula (I-A) is of formula:

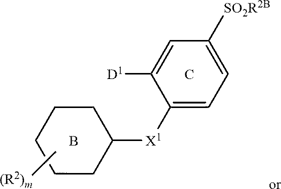

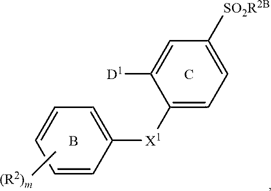

or a pharmaceutically acceptable salt, solvate, hydrate, polymorph, co-crystal, tautomer, stereoisomer, isotopically labeled derivative, or prodrug thereof.

In certain embodiments, the compound of Formula (I-A) is of formula:

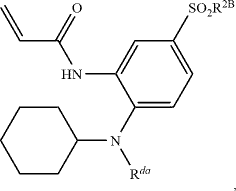

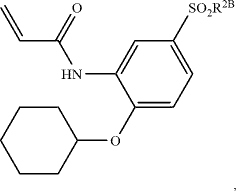

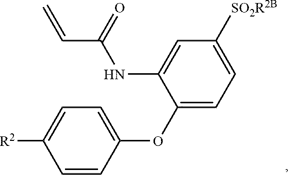

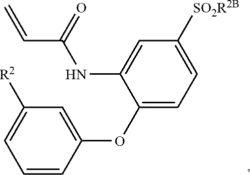

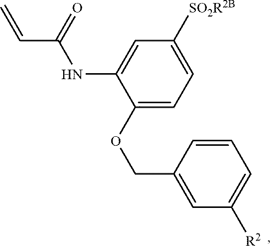

or a pharmaceutically acceptable salt, solvate, hydrate, polymorph, co-crystal, tautomer, stereoisomer, isotopically labeled derivative, or prodrug thereof.

In certain embodiments, the compound of Formula (I-A) is of formula:

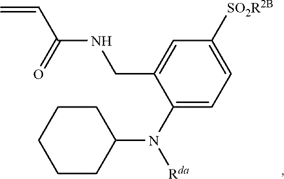

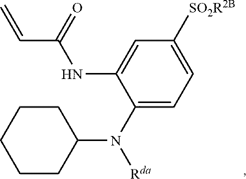

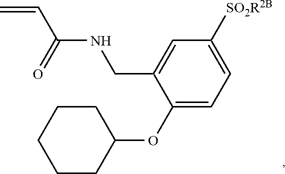

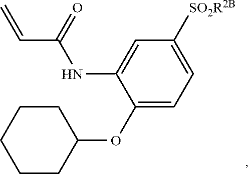

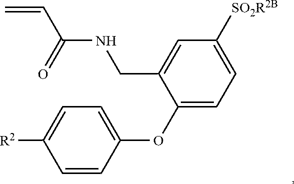

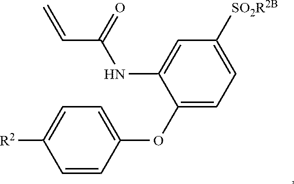

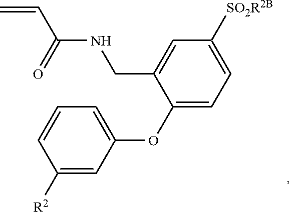

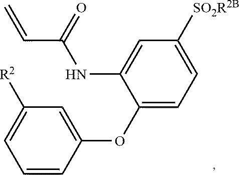

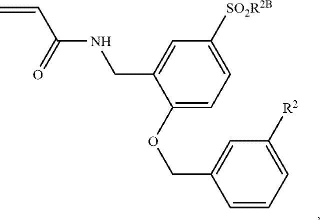

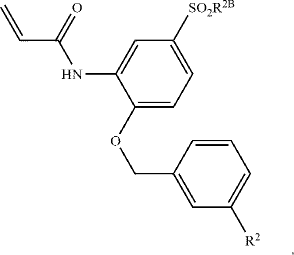

or a pharmaceutically acceptable salt, solvate, hydrate, polymorph, co-crystal, tautomer, stereoisomer, isotopically labeled derivative, or prodrug thereof.

In certain embodiments, the compound of Formula (I-A) is of formula:

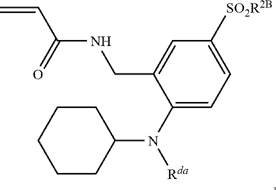

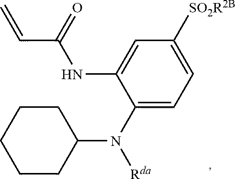

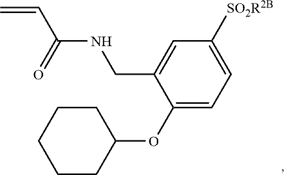

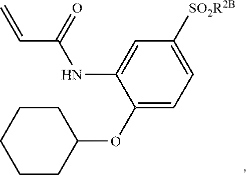

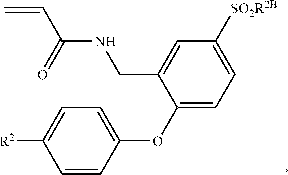

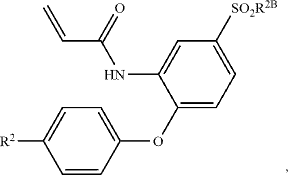

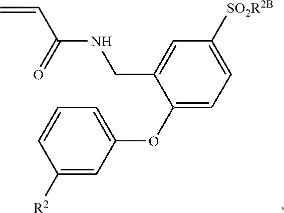

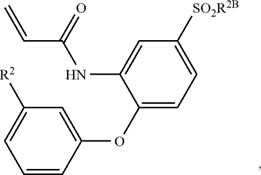

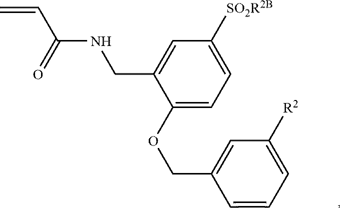

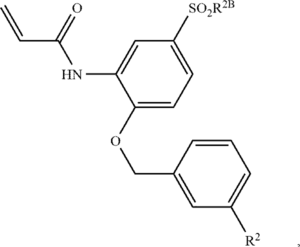

or a pharmaceutically acceptable salt thereof.

In certain embodiments, the compound of Formula (I-A) is of formula:

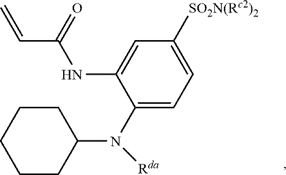

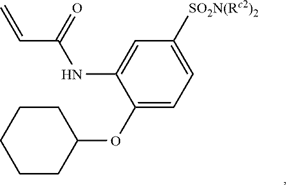

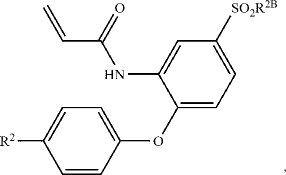

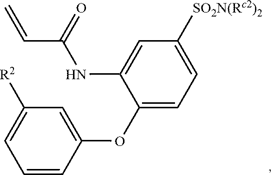

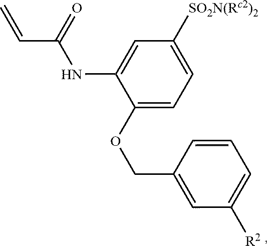

or a pharmaceutically acceptable salt, solvate, hydrate, polymorph, co-crystal, tautomer, stereoisomer, isotopically labeled derivative, or prodrug thereof.

In certain embodiments, the compound of Formula (I-A) is of formula:

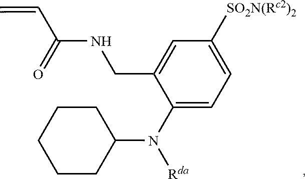

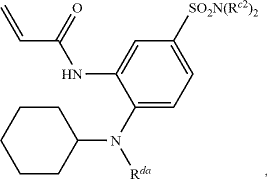

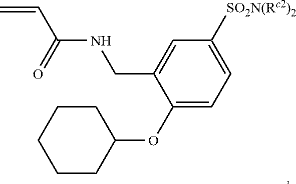

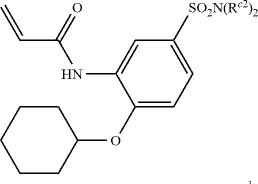

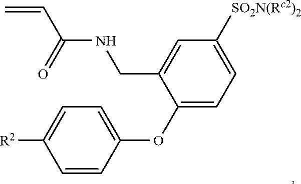

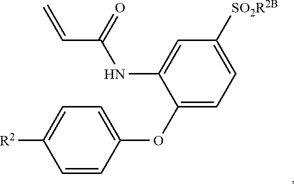

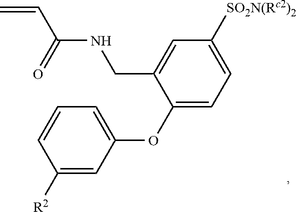

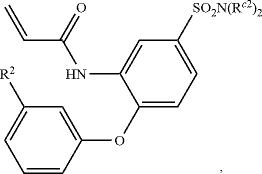

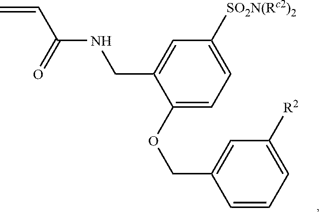

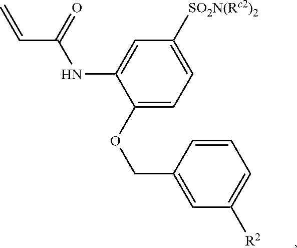

or a pharmaceutically acceptable salt, solvate, hydrate, polymorph, co-crystal, tautomer, stereoisomer, isotopically labeled derivative, or prodrug thereof.

In certain embodiments, the compound is of formula:

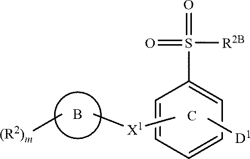

(I-A), or a pharmaceutically acceptable salt thereof.

In certain embodiments, the compound of Formula (I-A) is of formula:

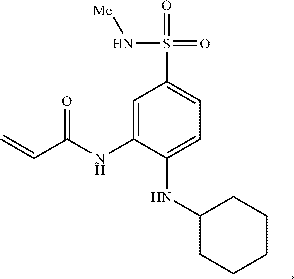

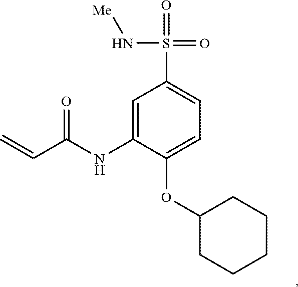

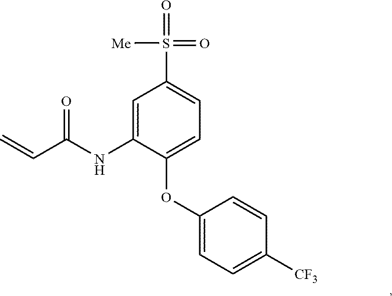

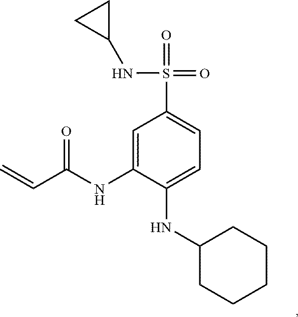

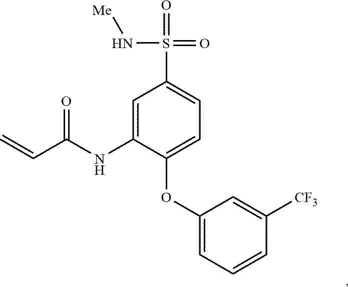

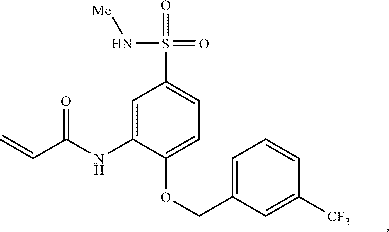

or a pharmaceutically acceptable salt, co-crystal, tautomer, stereoisomer, solvate, hydrate, polymorph, isotopically enriched derivative, or prodrug thereof.

In certain embodiments, the compound of Formula (I-A) is of formula:

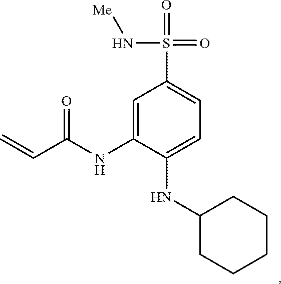

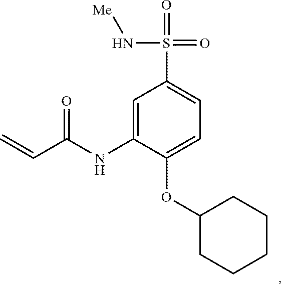

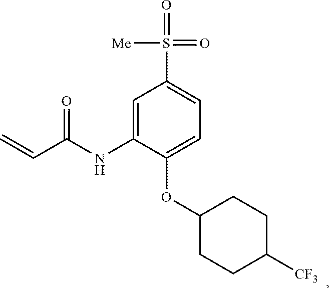

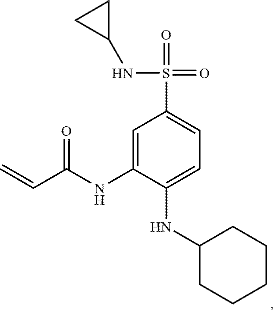

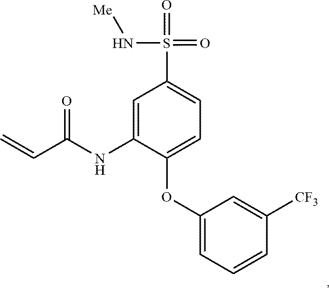

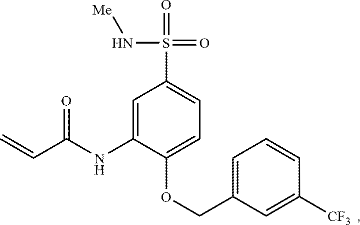

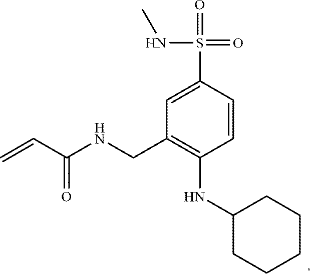

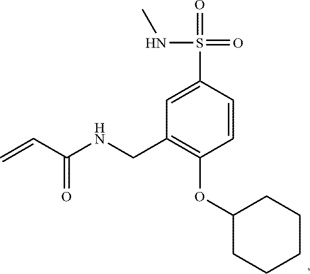

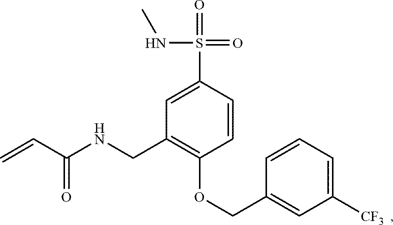

or a pharmaceutically acceptable salt, co-crystal, tautomer, stereoisomer, solvate, hydrate, polymorph, isotopically enriched derivative, or prodrug thereof.

In certain embodiments, the compound of Formula (I-A) is of formula:

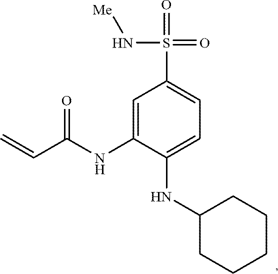

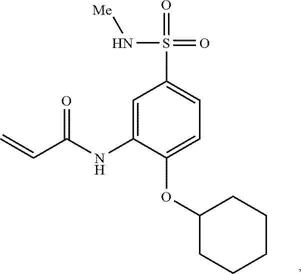

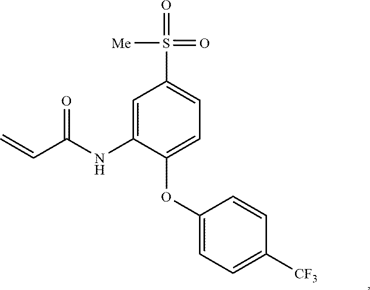

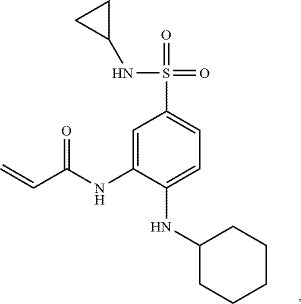

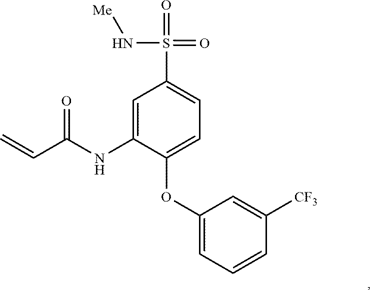

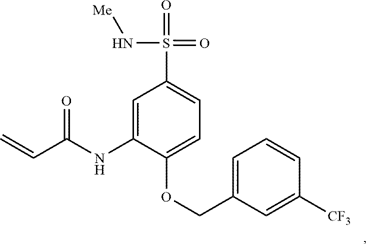

or a pharmaceutically acceptable salt thereof.

In certain embodiments, the compound of Formula (I-A) is of formula:

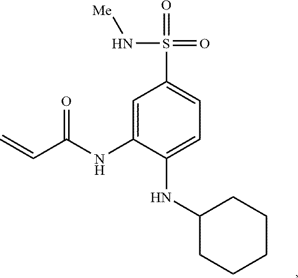

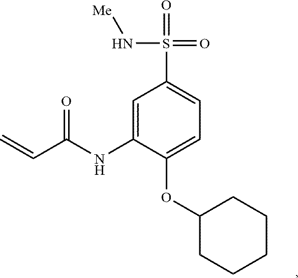

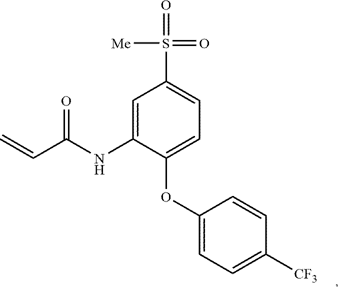

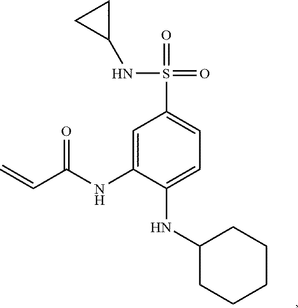

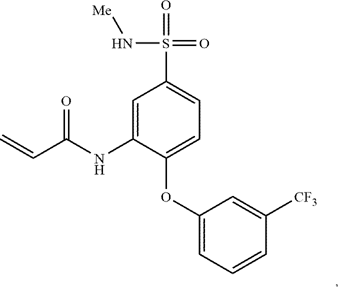

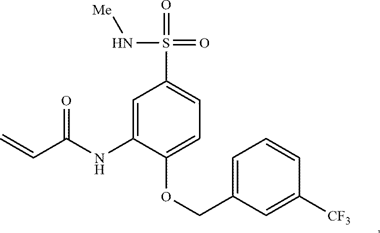

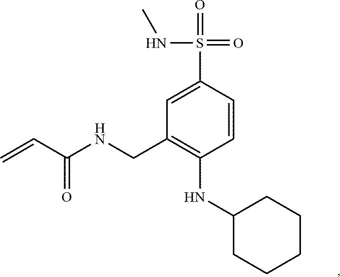

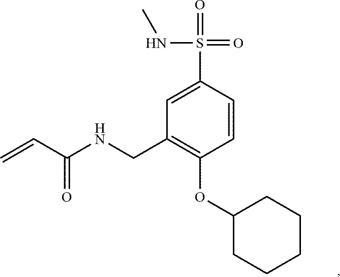

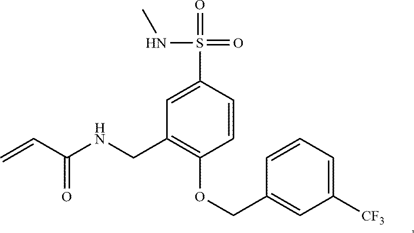

or a pharmaceutically acceptable salt thereof.

In certain embodiments, the compound of Formula (I-A) is not a compound disclosed in PCT Application No. PCT/US2019/056347, filed Oct. 15, 2019, PCT Application Publication No. WO 2018/204532, published Nov. 8, 2018, or PCT Application Publication No. WO 2019/040380, published Feb. 28, 2019. In certain embodiments, the compound of Formula (I-A) is not a compound disclosed in PCT Application No. PCT/US2019/056347, filed Oct. 15, 2019, and published as PCT Application Publication No. WO2020/081572 on Apr. 23, 2020 (e.g., the compound of Formula (I-A) is not a compound of claim 183 or of Examples 1-6 in PCT/US2019/056347 and WO2020/081572); PCT Application Publication No. WO 2018/204532 (e.g., the compound of Formula (I-A) is not a compound of Tables 1-3, Examples 1-252 or A1 of WO 2018/204532), published Nov. 8, 2018; or PCT Application Publication No. WO 2019/040380 (e.g., the compound of Formula (I-A) is not a compound of Tables 1-2, Examples 1-141 or A1 of WO 2019/040380), published Feb. 28, 2019. In certain embodiments, the compound of Formula (I-A) is not a compound disclosed in PCT Application No. PCT/US2019/056347, filed Oct. 15, 2019, and published as PCT Application Publication No. WO2020/081572 on Apr. 23, 2020 (e.g., the compound of Formula (I-A) is not a compound of claim 183 or of Examples 1-6 in PCT/US2019/056347 and WO2020/081572:

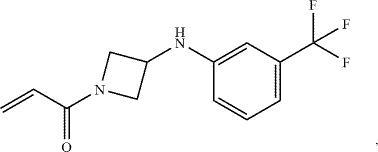

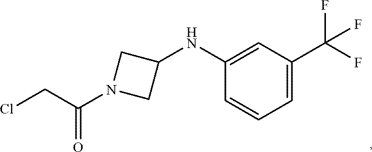

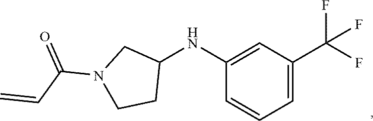

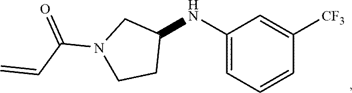

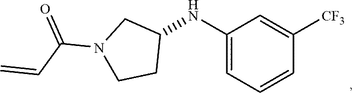

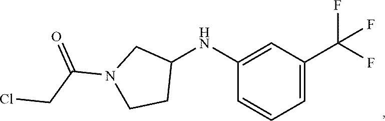

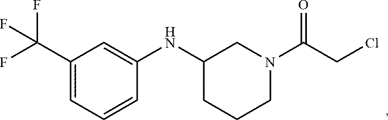

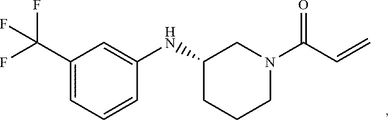

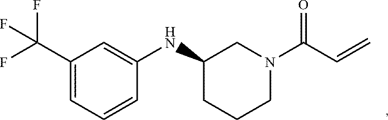

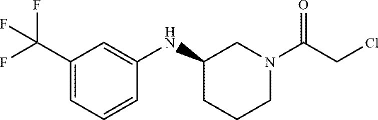 [SMILES: O=C(CCl)N1CCC[C@@H](Nc2cccc(C(F)(F)F)c2)C1]

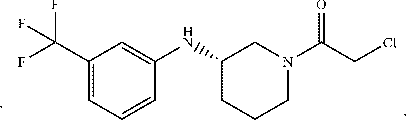

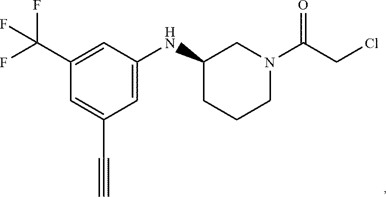

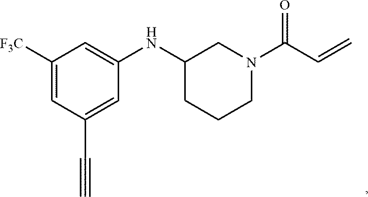

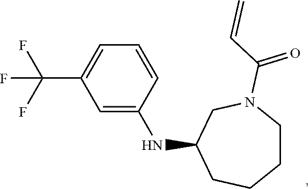

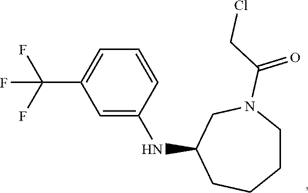

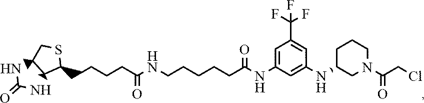

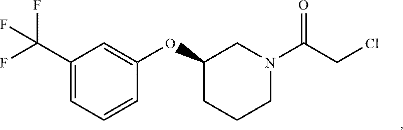

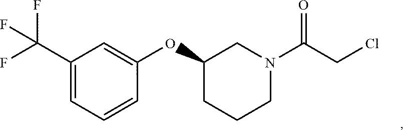

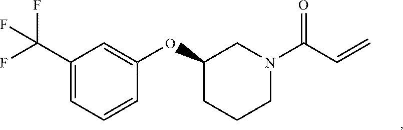

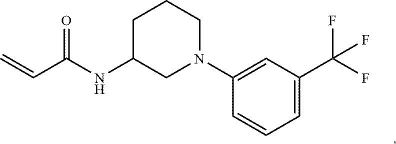

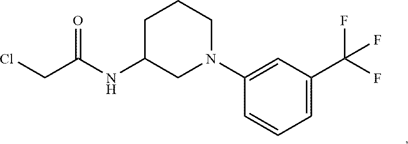

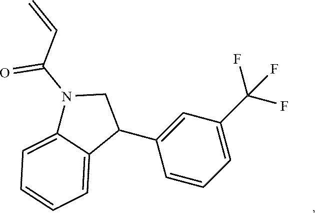

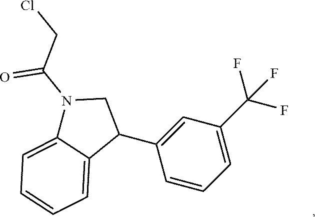

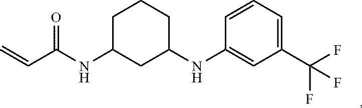

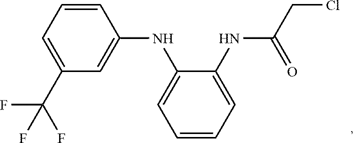

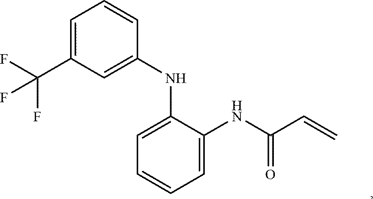

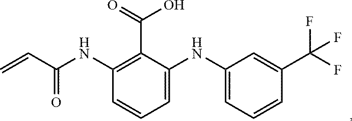

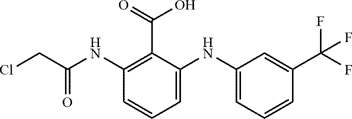 [SMILES: O=C(CCl)Nc1cccc(Nc2cccc(C(F)(F)F)c2)c1C(=O)O]

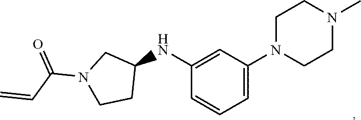

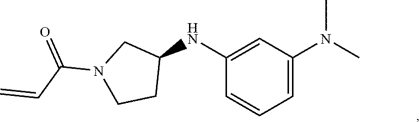

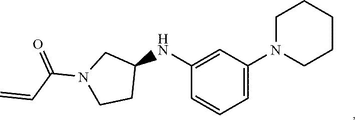

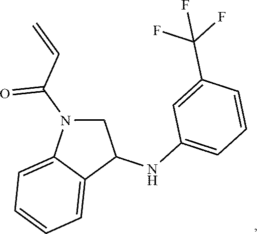

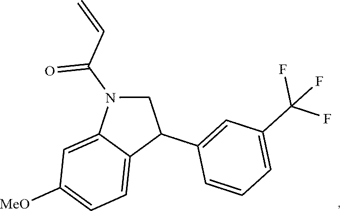

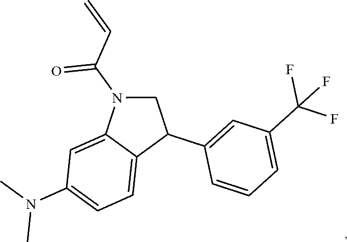

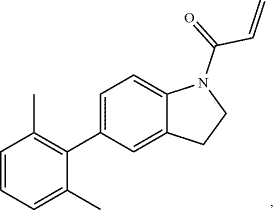

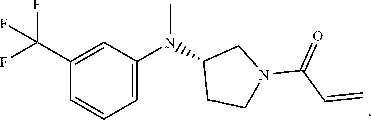

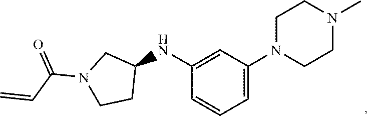

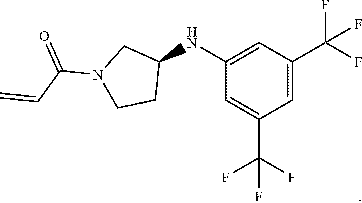

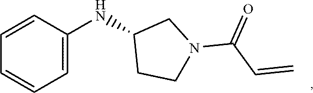

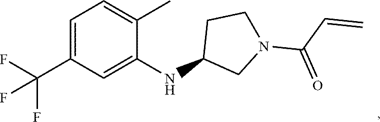

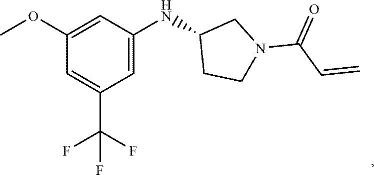

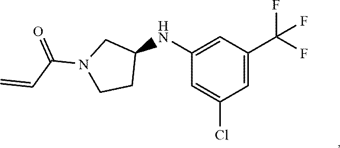

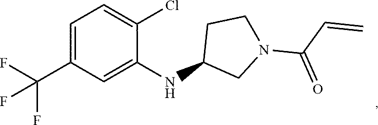

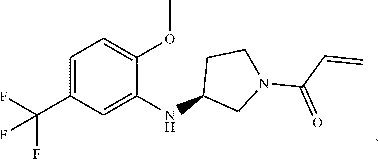

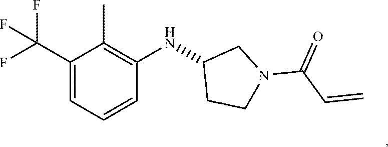

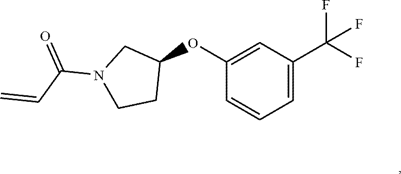

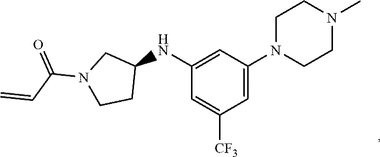

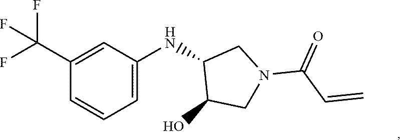

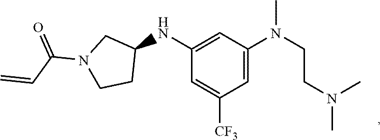

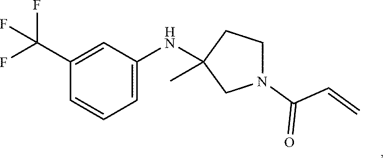

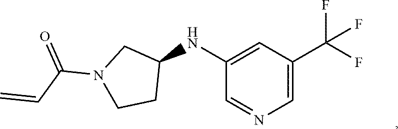

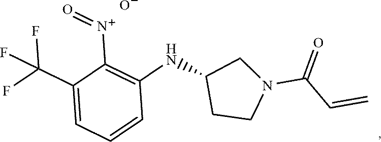

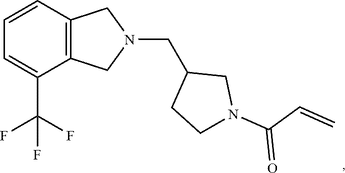

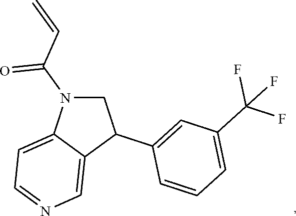

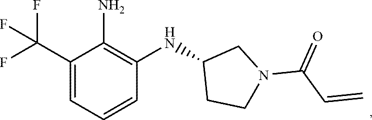

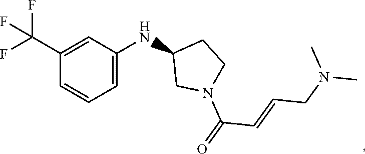

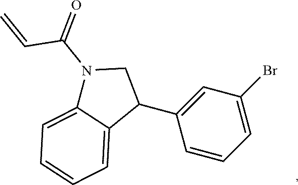

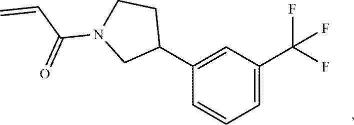

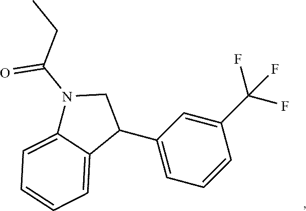

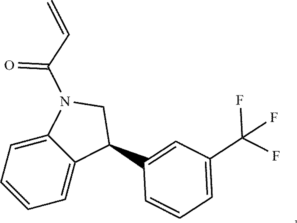

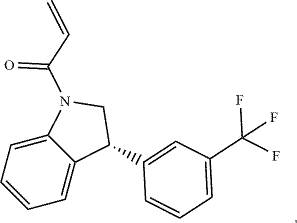

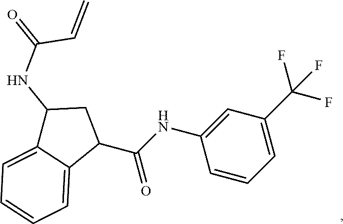

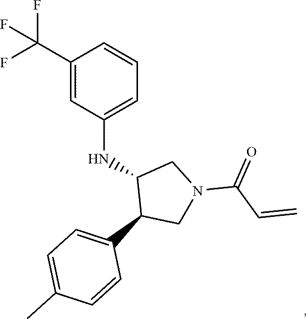

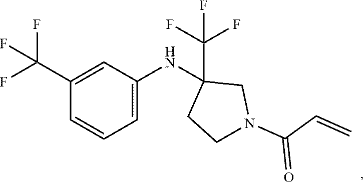

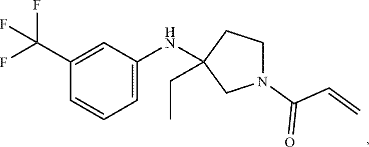

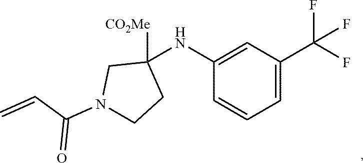

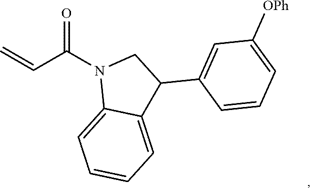

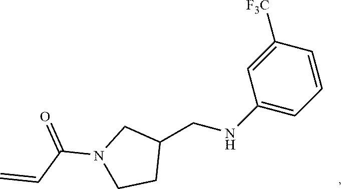

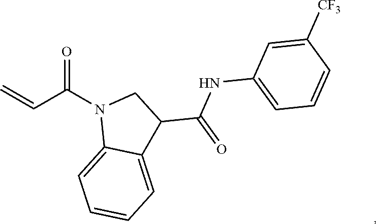

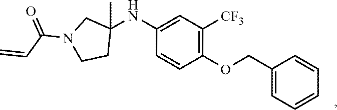

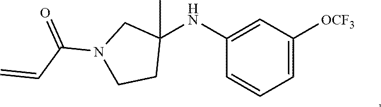

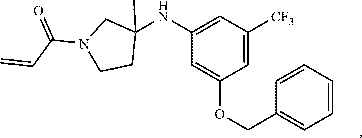

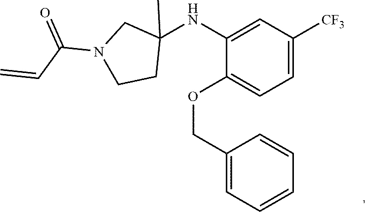

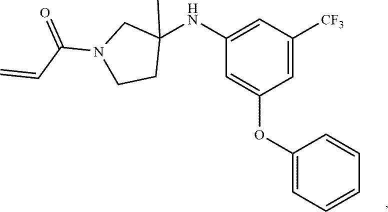

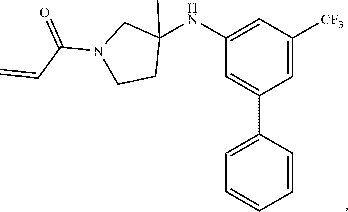

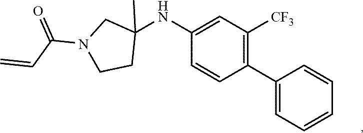

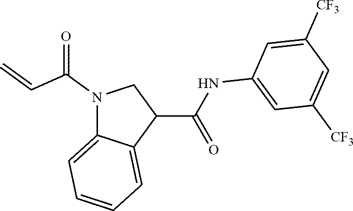 [SMILES: C=CC(=O)N1CC(C(=O)Nc2cc(C(F)(F)F)cc(C(F)(F)F)c2)c2ccccc21]

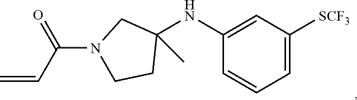

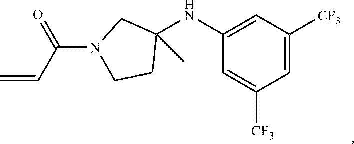

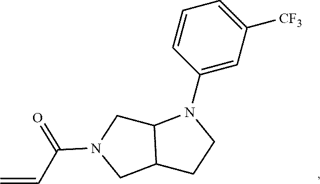

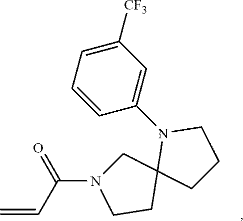

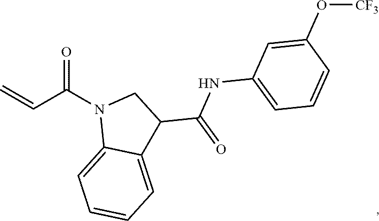

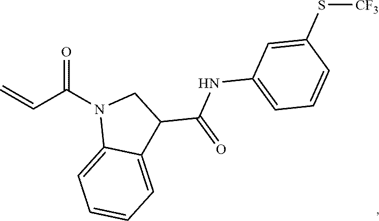

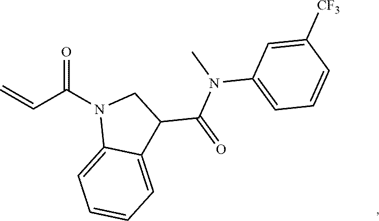

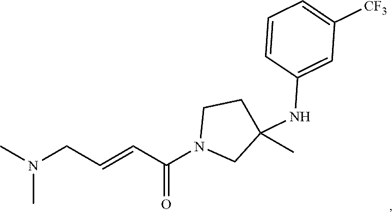

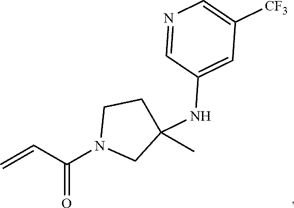

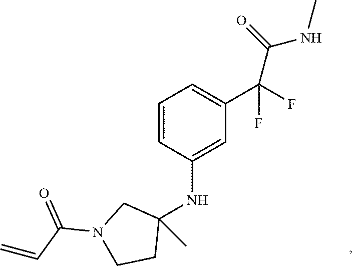

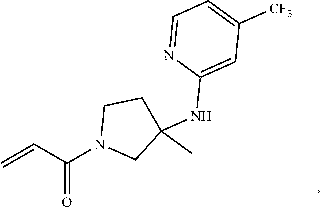

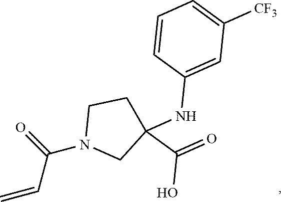

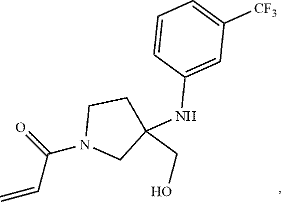

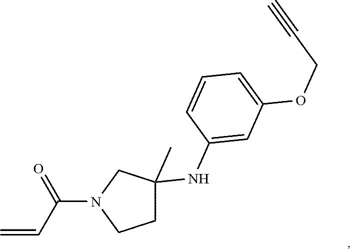

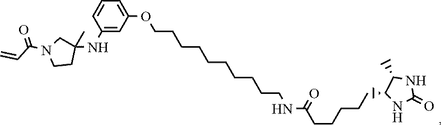

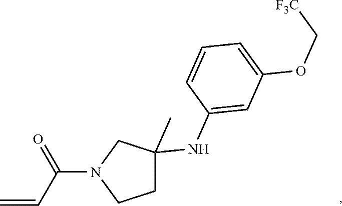

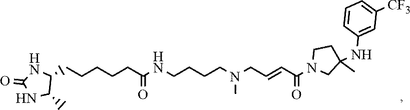

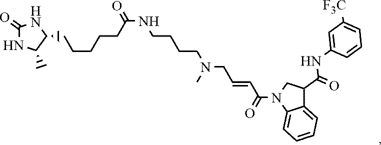

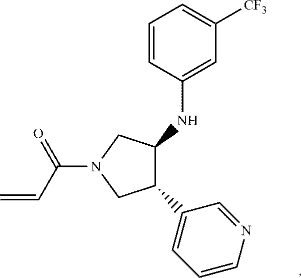

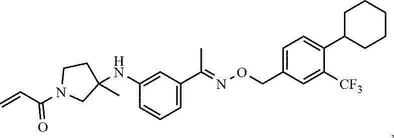

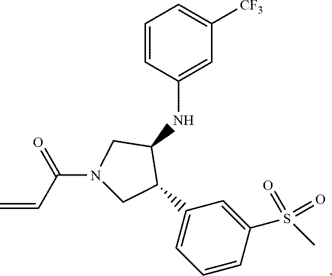

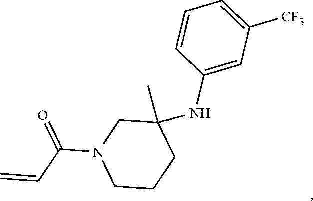

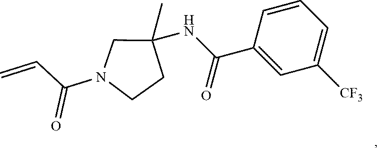

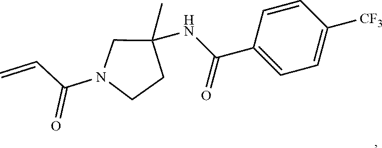

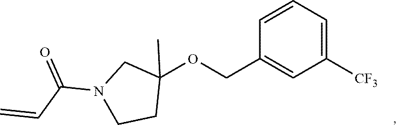

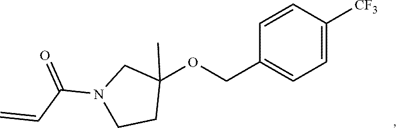

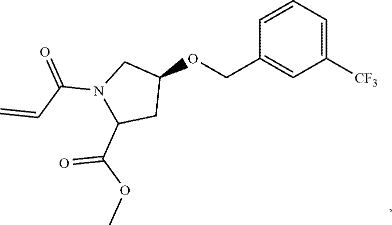

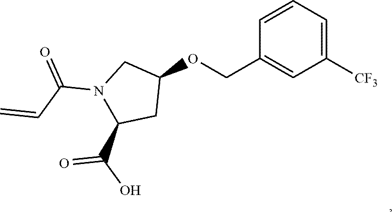

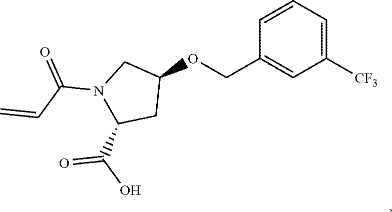

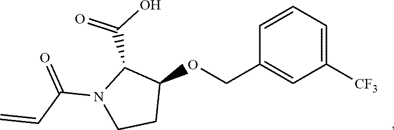

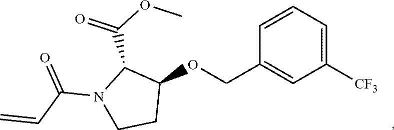

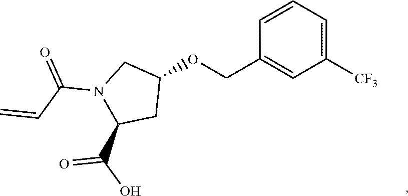

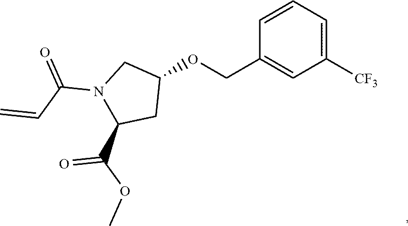

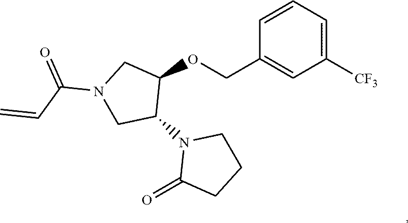

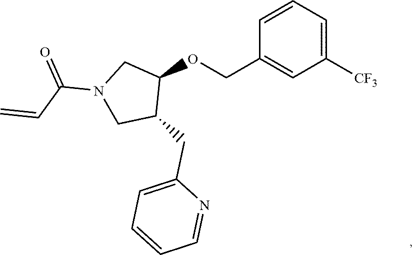

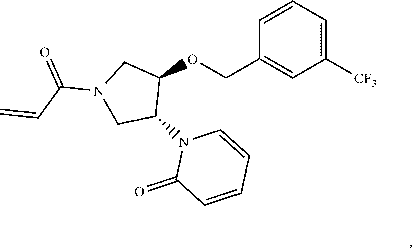

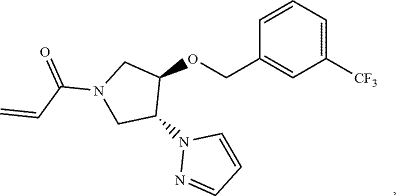

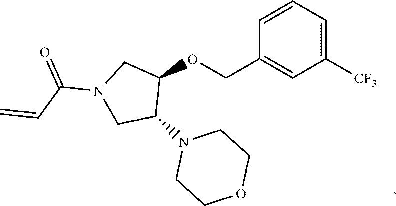

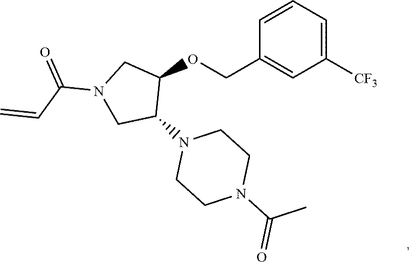

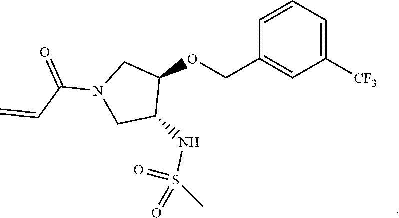

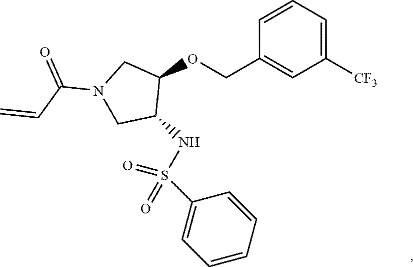

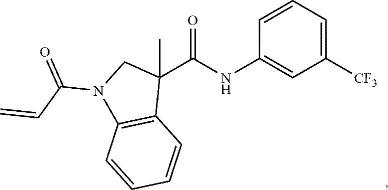

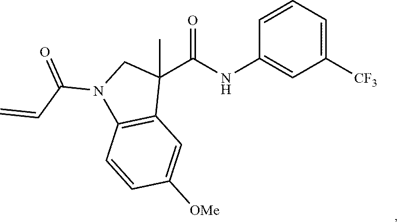

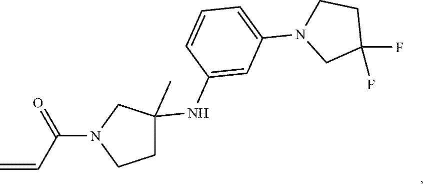

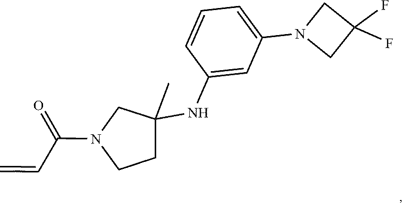

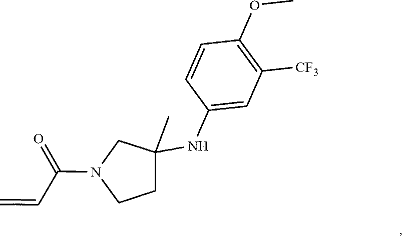

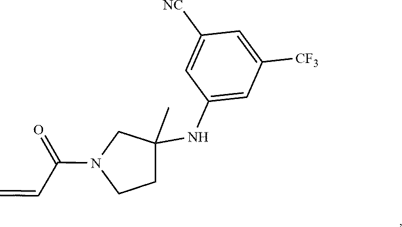

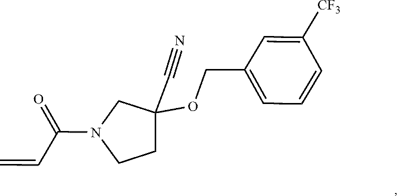

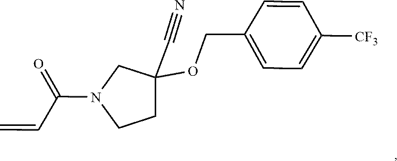

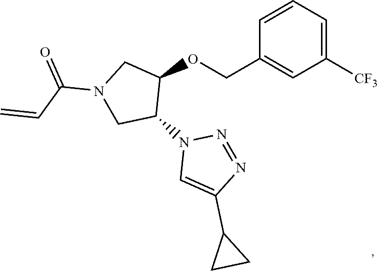

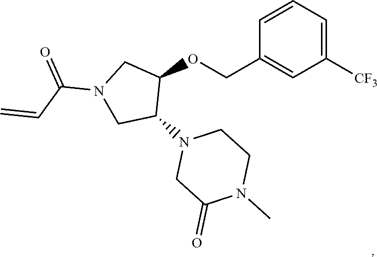

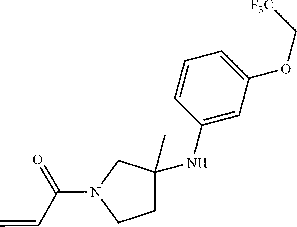

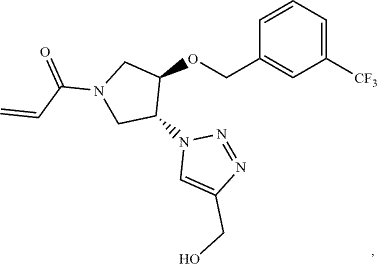

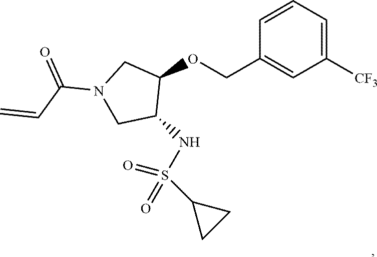

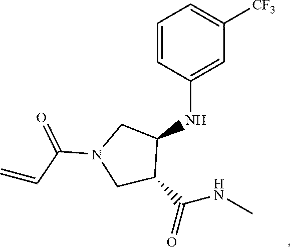

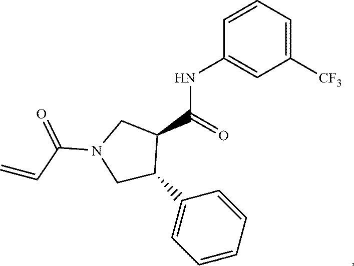

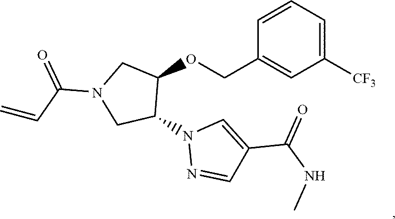

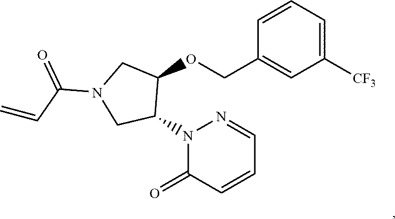

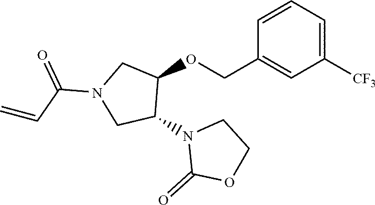 [SMILES: C=CC(=O)N1C[C@@H](N2CCOC2=O)[C@H](OCc2cccc(C(F)(F)F)c2)C1]

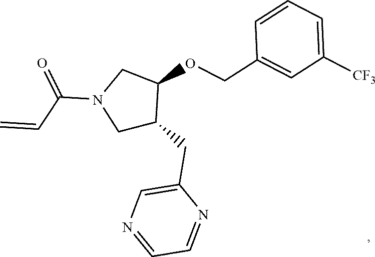

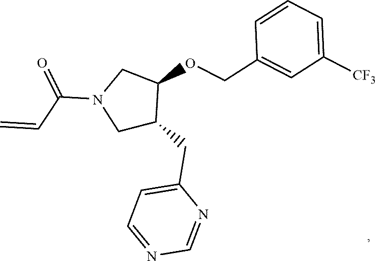

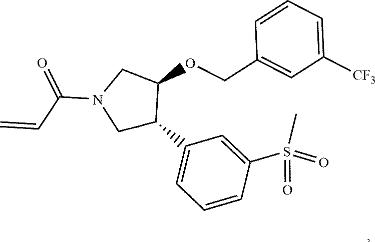

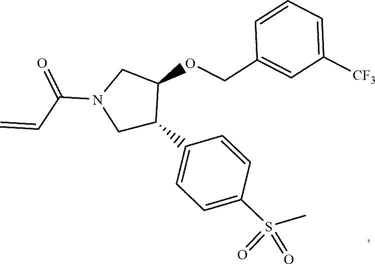

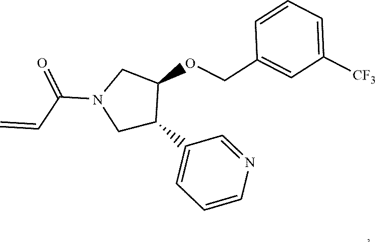

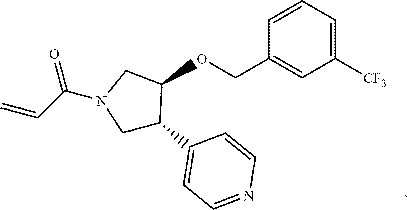

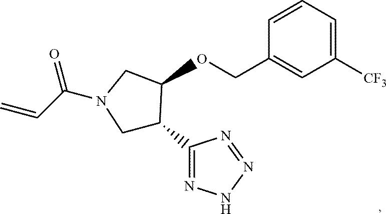

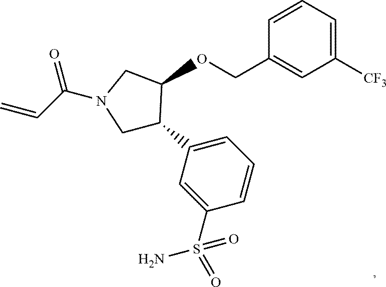

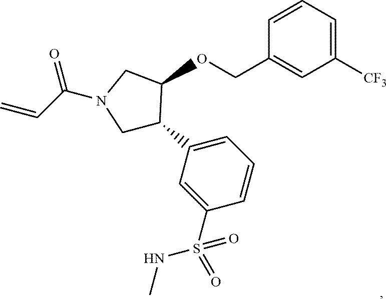

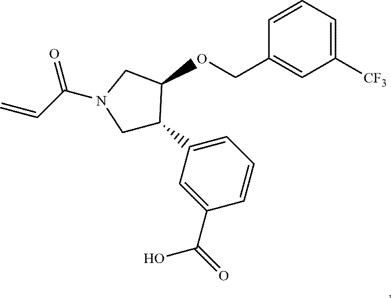

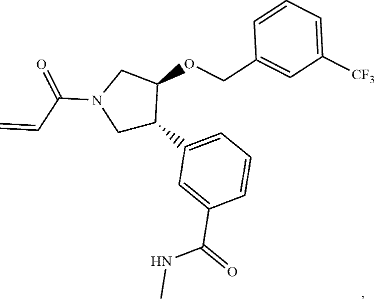

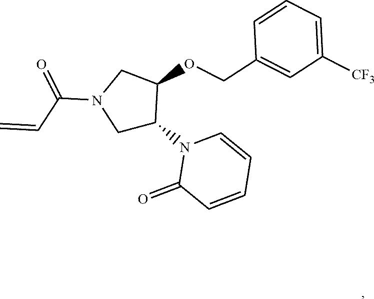

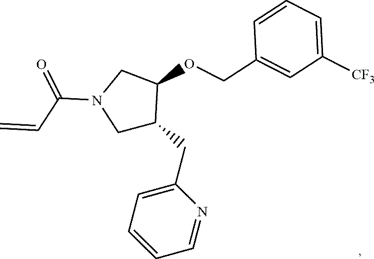

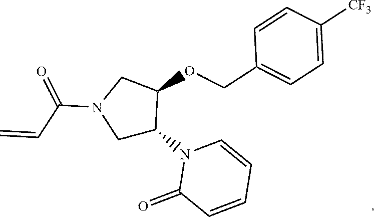

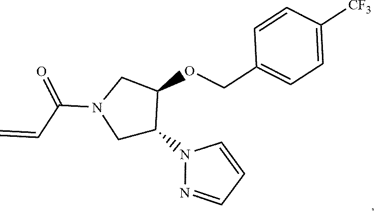

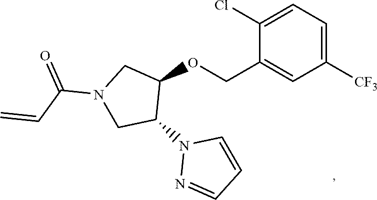

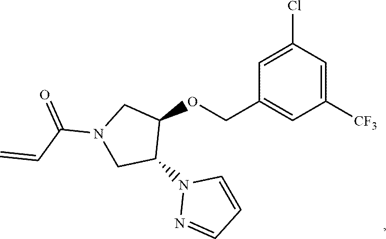

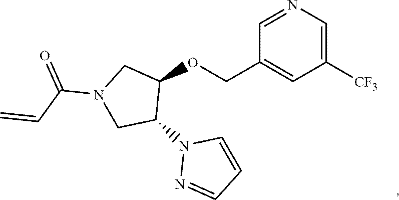

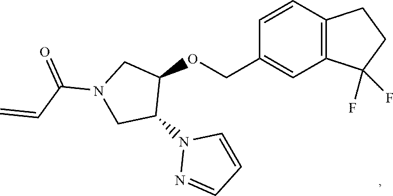

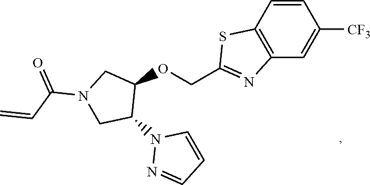

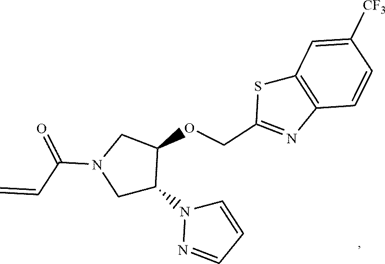

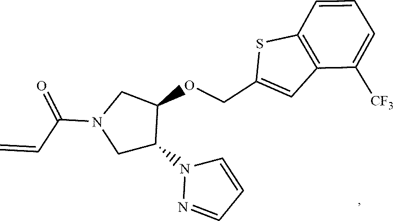

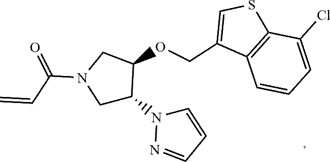

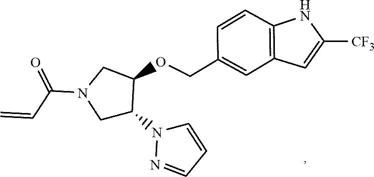

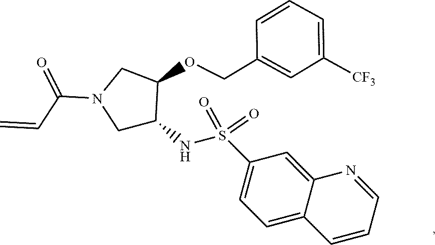

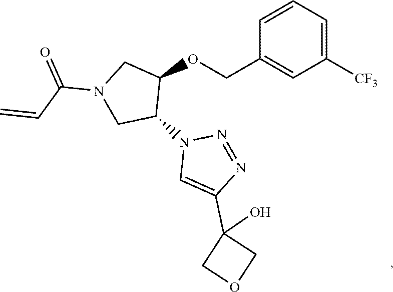

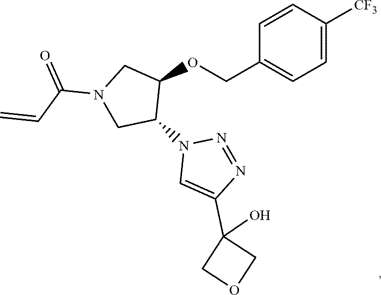

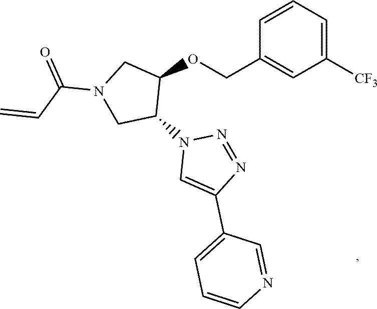

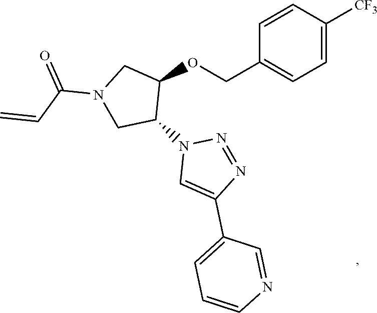

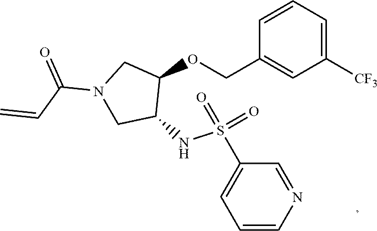

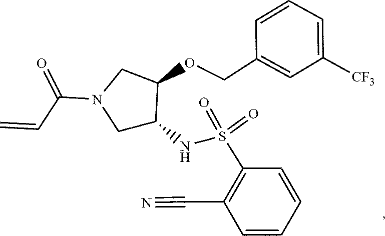

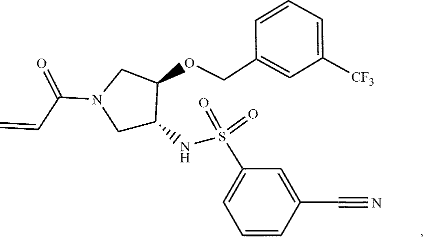

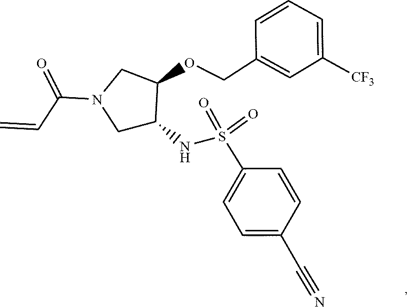

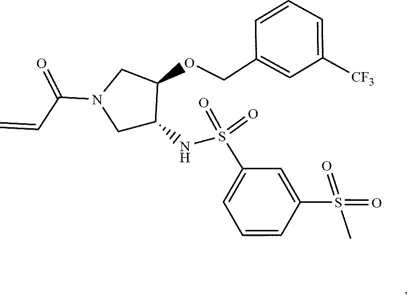

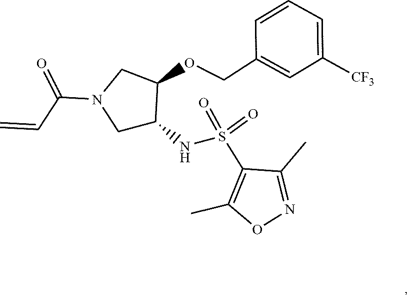

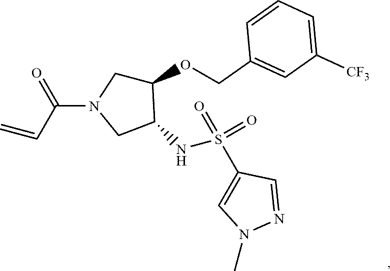

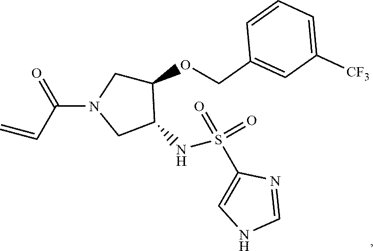

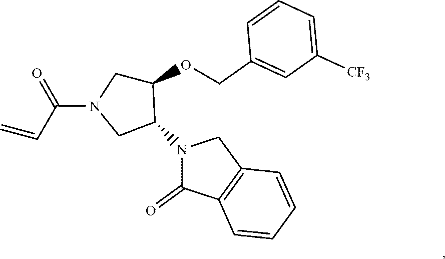

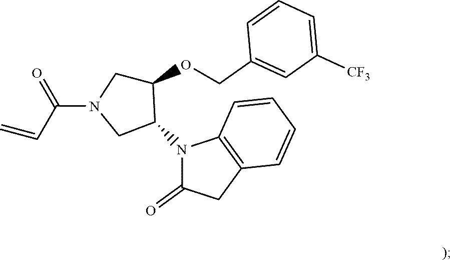

PCT Application Publication No. WO 2018/204532 (e.g., the compound of Formula (I-A) is not a compound of Tables 1-3, Examples 1-252 or A1 of WO 20181204532:

| Compound No. | Structure |
|---|---|
| 1 | 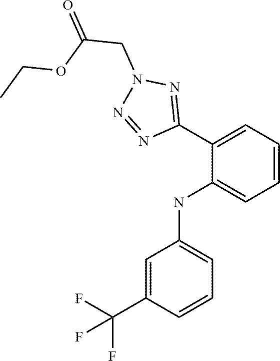 [SMILES: CCOC(=O)Cn1nnc(-c2ccccc2Nc2cccc(C(F)(F)F)c2)n1] |
| 2 | 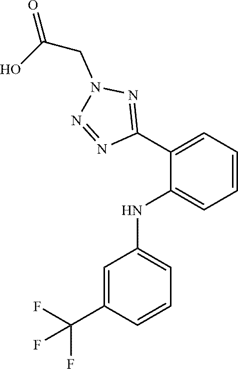 [SMILES: O=C(O)Cn1nnc(-c2ccccc2Nc2cccc(C(F)(F)F)c2)n1] |
| 3 | 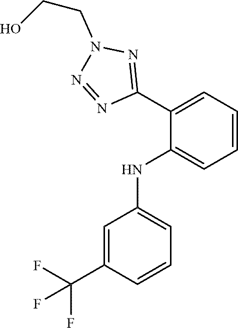 [SMILES: OCCn1nnc(-c2ccccc2Nc2cccc(C(F)(F)F)c2)n1] |

| | |
|---|---|
| 4 | 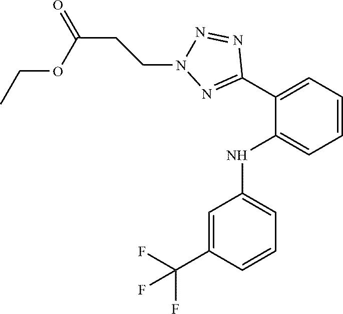 [SMILES: CCOC(=O)CCn1nnc(-c2ccccc2Nc2cccc(C(F)(F)F)c2)n1] |
| 5 | 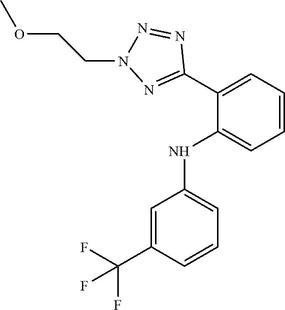 [SMILES: COCCn1nnc(-c2ccccc2Nc2cccc(C(F)(F)F)c2)n1] |

| Compound No. | Structure |
|---|---|
| 6 | 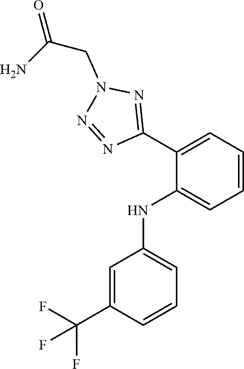 [SMILES: NC(=O)Cn1nnc(-c2ccccc2Nc2cccc(C(F)(F)F)c2)n1] |
| 7 | 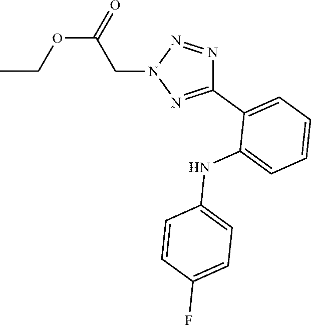 [SMILES: CCOC(=O)Cn1nnc(-c2ccccc2Nc2ccc(F)cc2)n1] |
| 8 | 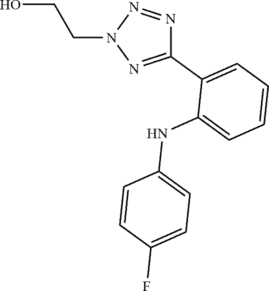 [SMILES: OCCn1nnc(-c2ccccc2Nc2ccc(F)cc2)n1] |
| 9 | 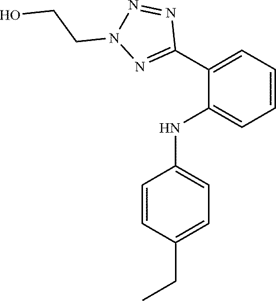 [SMILES: CCc1ccc(Nc2ccccc2-c2nnn(CCO)n2)cc1] |
| 10 | 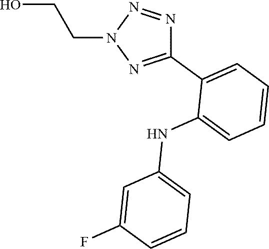 [SMILES: OCCn1nnc(-c2ccccc2Nc2cccc(F)c2)n1] |

| | |
|---|---|
| 11 | 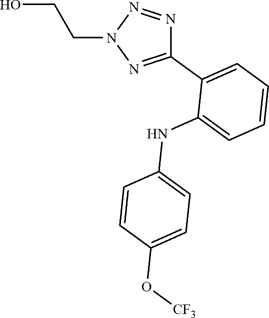 [SMILES: OCCn1nnc(-c2ccccc2Nc2ccc(OC(F)(F)F)cc2)n1] |

| Compound No. | Structure |
|---|---|
| 12 | 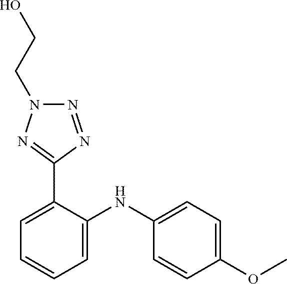 [SMILES: COc1ccc(Nc2ccccc2-c2nnn(CCO)n2)cc1] |
| 13 | 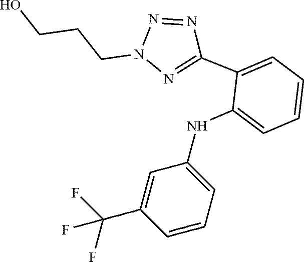 [SMILES: OCCCn1nnc(-c2ccccc2Nc2cccc(C(F)(F)F)c2)n1] |

| | |
|---|---|
| 14 | 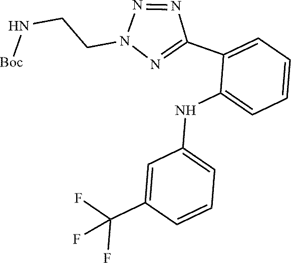 [SMILES: CC(C)(C)OC(=O)NCCn1nnc(-c2ccccc2Nc2cccc(C(F)(F)F)c2)n1] |
| 15 | 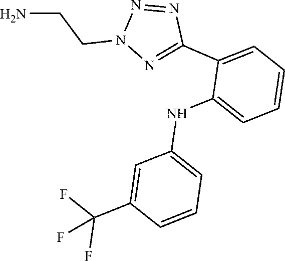 [SMILES: NCCn1nnc(-c2ccccc2Nc2cccc(C(F)(F)F)c2)n1] |

| | |
|---|---|
| 16 | 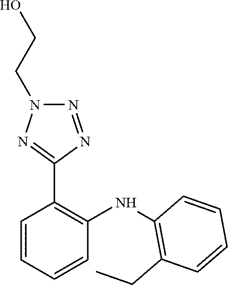 [SMILES: CCc1ccccc1Nc1ccccc1-c1nnn(CCO)n1] |
| 17 | 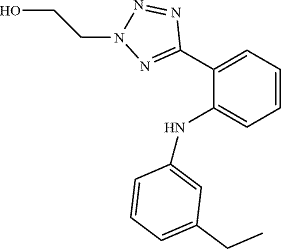 [SMILES: CCc1cccc(Nc2ccccc2-c2nnn(CCO)n2)c1] |

| | |
|---|---|
| 18 | 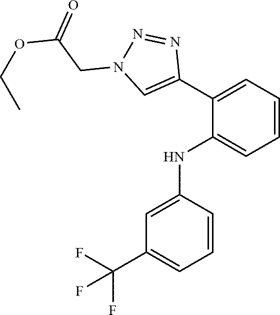 [SMILES: CCOC(=O)Cn1cc(-c2ccccc2Nc2cccc(C(F)(F)F)c2)nn1] |
| 19 | 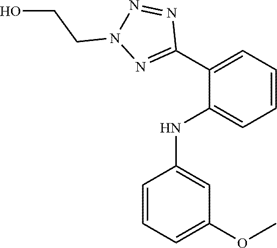 [SMILES: COc1cccc(Nc2ccccc2-c2nnn(CCO)n2)c1] |
| 20 | 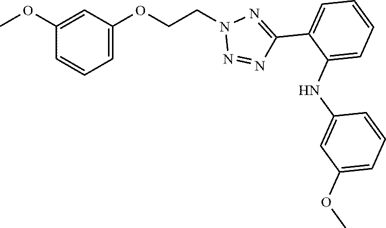 [SMILES: COc1cccc(Nc2ccccc2-c2nnn(CCOc3cccc(OC)c3)n2)c1] |

| | |
|---|---|
| 21 | 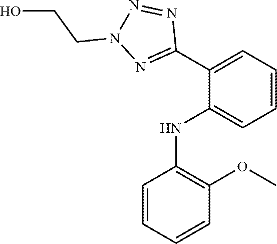 [SMILES: COc1ccccc1Nc1ccccc1-c1nnn(CCO)n1] |
| 22 | 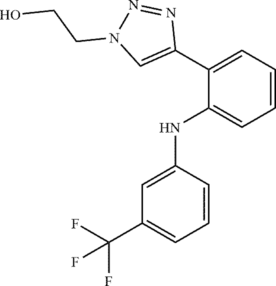 [SMILES: OCCn1cc(-c2ccccc2Nc2cccc(C(F)(F)F)c2)nn1] |
| 23 | 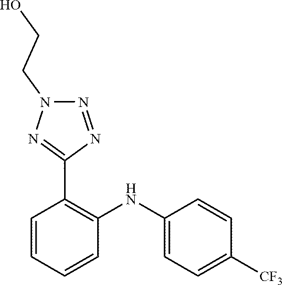 [SMILES: OCCn1nnc(-c2ccccc2Nc2ccc(C(F)(F)F)cc2)n1] |

| | |
|---|---|
| 24 | 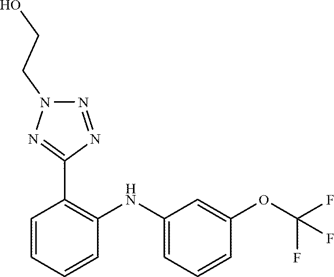 [SMILES: OCCn1nnc(-c2ccccc2Nc2cccc(OC(F)(F)F)c2)n1] |
| 25 | 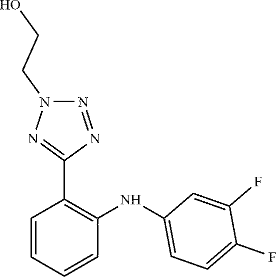 [SMILES: OCCn1nnc(-c2ccccc2Nc2ccc(F)c(F)c2)n1] |
| 26 | 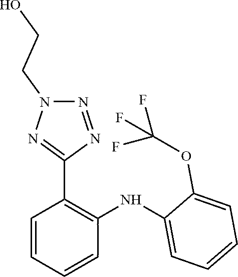 [SMILES: OCCn1nnc(-c2ccccc2Nc2ccccc2OC(F)(F)F)n1] |

| | |
|---|---|
| 27 | 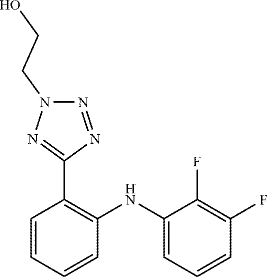 [SMILES: OCCn1nnc(-c2ccccc2Nc2cccc(F)c2F)n1] |
| 28 | 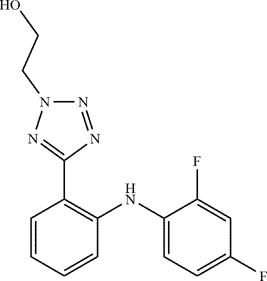 [SMILES: OCCn1nnc(-c2ccccc2Nc2ccc(F)cc2F)n1] |
| 29 | 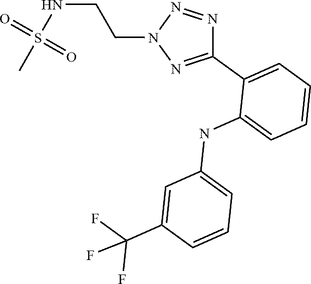 [SMILES: CS(=O)(=O)NCCn1nnc(-c2ccccc2Nc2cccc(C(F)(F)F)c2)n1] |

| | |
|---|---|
| 30 | 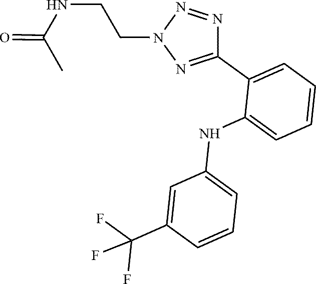 [SMILES: CC(=O)NCCn1nnc(-c2ccccc2Nc2cccc(C(F)(F)F)c2)n1] |
| 31 | 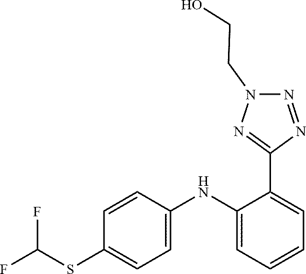 [SMILES: OCCn1nnc(-c2ccccc2Nc2ccc(SC(F)F)cc2)n1] |
| 32 | 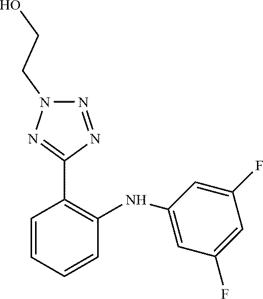 [SMILES: OCCn1nnc(-c2ccccc2Nc2cc(F)cc(F)c2)n1] |
| 33 | 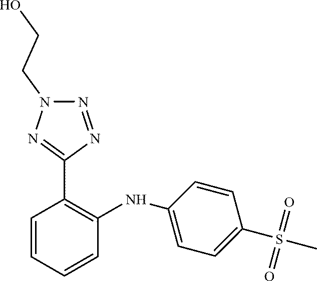 [SMILES: CS(=O)(=O)c1ccc(Nc2ccccc2-c2nnn(CCO)n2)cc1] |
| 34 | 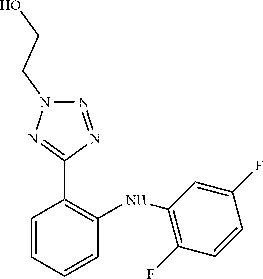 [SMILES: OCCn1nnc(-c2ccccc2Nc2cc(F)ccc2F)n1] |
| 35 | 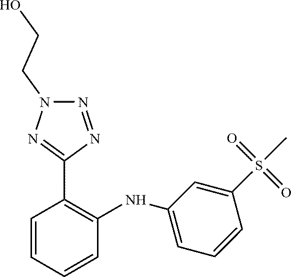 [SMILES: CS(=O)(=O)c1cccc(Nc2ccccc2-c2nnn(CCO)n2)c1] |

| | |
|---|---|
| 36 | 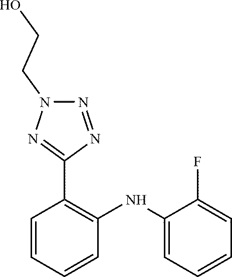 [SMILES: OCCn1nnc(-c2ccccc2Nc2ccccc2F)n1] |
| 37 | 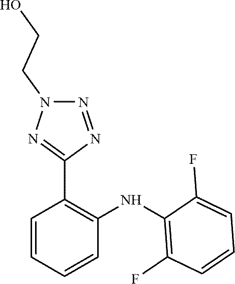 [SMILES: OCCn1nnc(-c2ccccc2Nc2c(F)cccc2F)n1] |
| 38 | 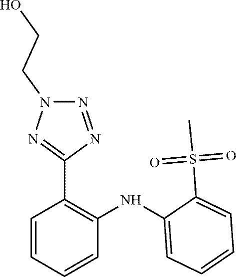 [SMILES: CS(=O)(=O)c1ccccc1Nc1ccccc1-c1nnn(CCO)n1] |
| 39 | 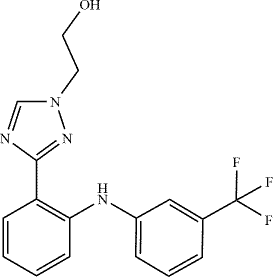 [SMILES: OCCn1cnc(-c2ccccc2Nc2cccc(C(F)(F)F)c2)n1] |
| 40 | 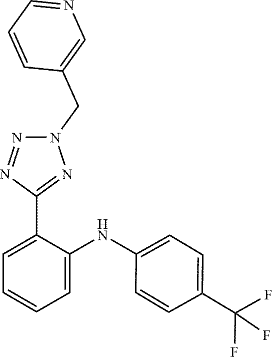 [SMILES: FC(F)(F)c1ccc(Nc2ccccc2-c2nnn(Cc3cccnc3)n2)cc1] |
| 41 | 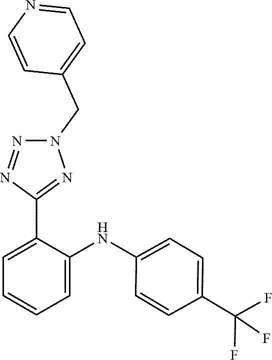 [SMILES: FC(F)(F)c1ccc(Nc2ccccc2-c2nnn(Cc3ccncc3)n2)cc1] |

| | |
|---|---|
| 42 | 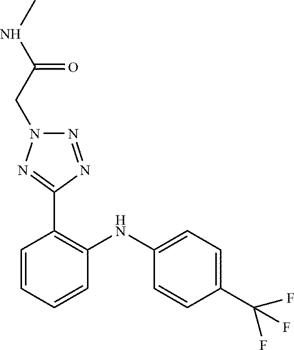 [SMILES: CNC(=O)Cn1nnc(-c2ccccc2Nc2ccc(C(F)(F)F)cc2)n1] |
| 43 | 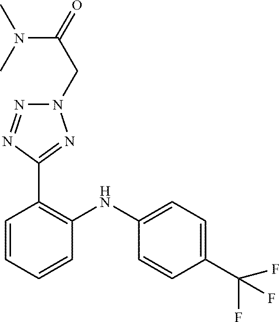 [SMILES: CN(C)C(=O)Cn1nnc(-c2ccccc2Nc2ccc(C(F)(F)F)cc2)n1] |
| 44 | 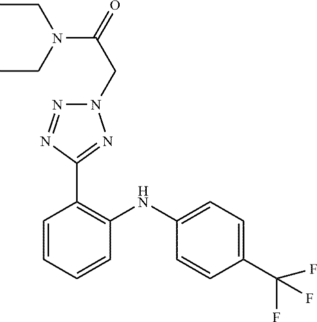 [SMILES: CCN(CC)C(=O)Cn1nnc(-c2ccccc2Nc2ccc(C(F)(F)F)cc2)n1] |
| 45 | 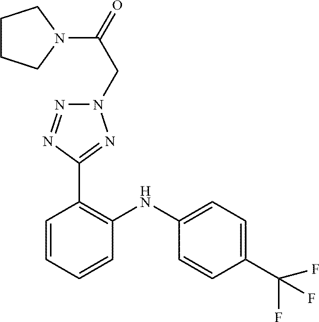 [SMILES: O=C(Cn1nnc(-c2ccccc2Nc2ccc(C(F)(F)F)cc2)n1)N1CCCC1] |
| 46 | 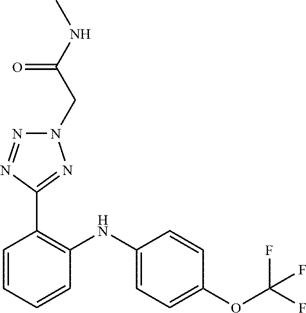 [SMILES: CNC(=O)Cn1nnc(-c2ccccc2Nc2ccc(OC(F)(F)F)cc2)n1] |

| | |
|---|---|
| 47 | 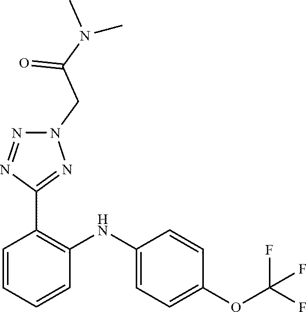 [SMILES: CN(C)C(=O)Cn1nnc(-c2ccccc2Nc2ccc(OC(F)(F)F)cc2)n1] |
| 48 | 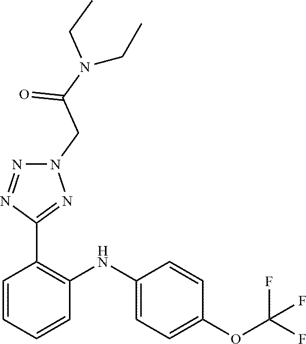 [SMILES: CCN(CC)C(=O)Cn1nnc(-c2ccccc2Nc2ccc(OC(F)(F)F)cc2)n1] |
| 49 | 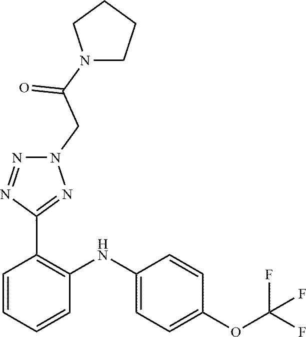 [SMILES: O=C(Cn1nnc(-c2ccccc2Nc2ccc(OC(F)(F)F)cc2)n1)N1CCCC1] |
| 50 | 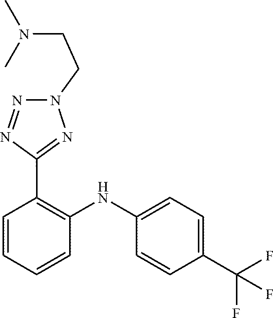 [SMILES: CN(C)CCn1nnc(-c2ccccc2Nc2ccc(C(F)(F)F)cc2)n1] |
| 51 | 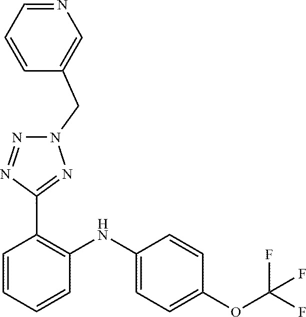 [SMILES: FC(F)(F)Oc1ccc(Nc2ccccc2-c2nnn(Cc3cccnc3)n2)cc1] |

| | |
|---|---|
| 52 | 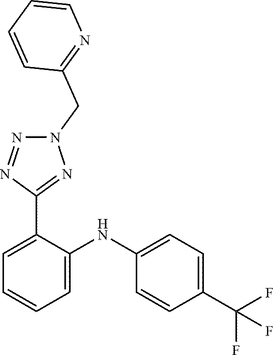 [SMILES: FC(F)(F)c1ccc(Nc2ccccc2-c2nnn(Cc3ccccn3)n2)cc1] |
| 53 | 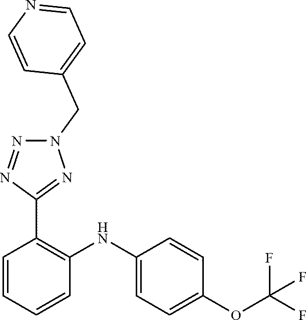 [SMILES: FC(F)(F)Oc1ccc(Nc2ccccc2-c2nnn(Cc3ccncc3)n2)cc1] |
| 54 | 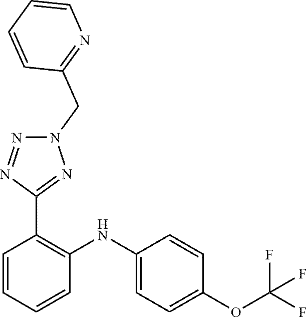 [SMILES: FC(F)(F)Oc1ccc(Nc2ccccc2-c2nnn(Cc3ccccn3)n2)cc1] |
| 55 | 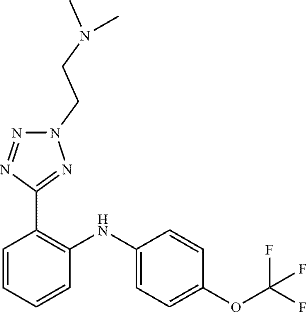 [SMILES: CN(C)CCn1nnc(-c2ccccc2Nc2ccc(OC(F)(F)F)cc2)n1] |
| 56 | 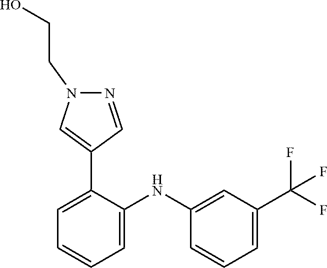 [SMILES: OCCn1cc(-c2ccccc2Nc2cccc(C(F)(F)F)c2)cn1] |

| | |
|---|---|
| 57 | 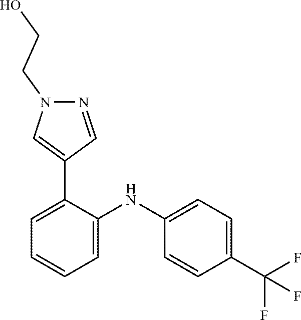 [SMILES: OCCn1cc(-c2ccccc2Nc2ccc(C(F)(F)F)cc2)cn1] |
| 58 | 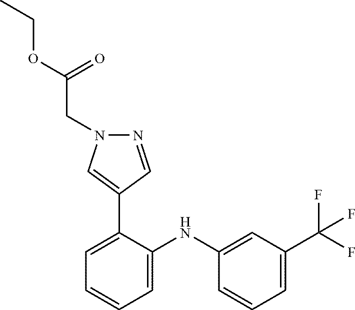 [SMILES: CCOC(=O)Cn1cc(-c2ccccc2Nc2cccc(C(F)(F)F)c2)cn1] |
| 59 | 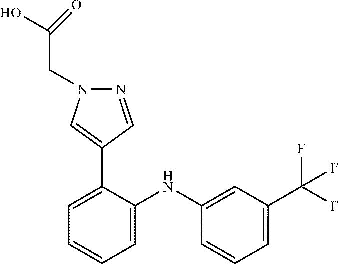 [SMILES: O=C(O)Cn1cc(-c2ccccc2Nc2cccc(C(F)(F)F)c2)cn1] |
| 60 | 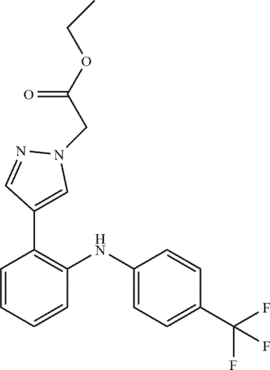 [SMILES: CCOC(=O)Cn1cc(-c2ccccc2Nc2ccc(C(F)(F)F)cc2)cn1] |
| 61 | 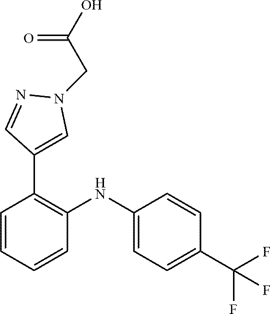 [SMILES: O=C(O)Cn1cc(-c2ccccc2Nc2ccc(C(F)(F)F)cc2)cn1] |

| | |
|---|---|
| 62 | 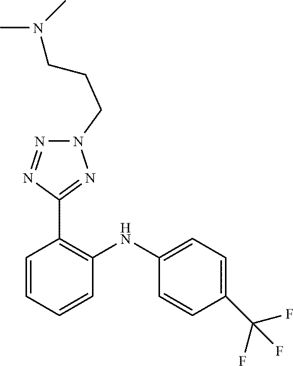 [SMILES: CN(C)CCCn1nnc(-c2ccccc2Nc2ccc(C(F)(F)F)cc2)n1] |
| 63 | 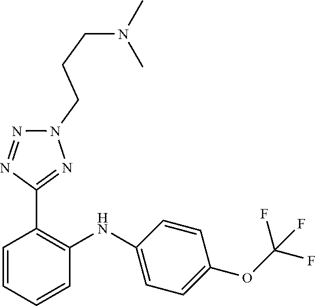 [SMILES: CN(C)CCCn1nnc(-c2ccccc2Nc2ccc(OC(F)(F)F)cc2)n1] |
| 64 | 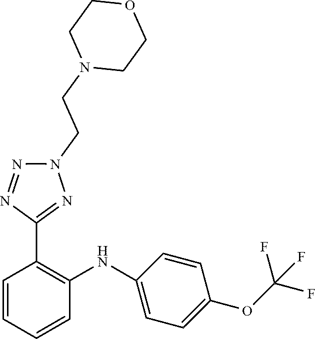 [SMILES: FC(F)(F)Oc1ccc(Nc2ccccc2-c2nnn(CCN3CCOCC3)n2)cc1] |
| 65 | 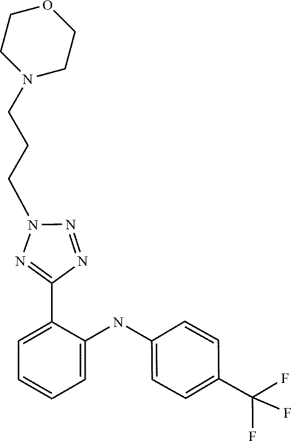 [SMILES: FC(F)(F)c1ccc(Nc2ccccc2-c2nnn(CCCN3CCOCC3)n2)cc1] |
| 66 | 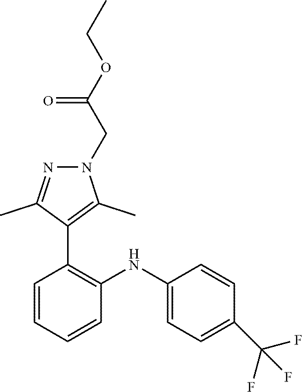 [SMILES: CCOC(=O)Cn1nc(C)c(-c2ccccc2Nc2ccc(C(F)(F)F)cc2)c1C] |

| | |
|---|---|
| 67 | 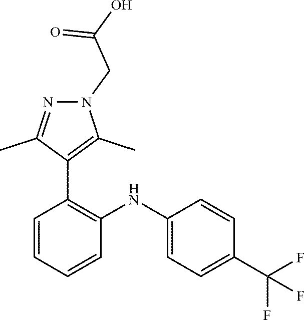 [SMILES: Cc1nn(CC(=O)O)c(C)c1-c1ccccc1Nc1ccc(C(F)(F)F)cc1] |
| 68 | 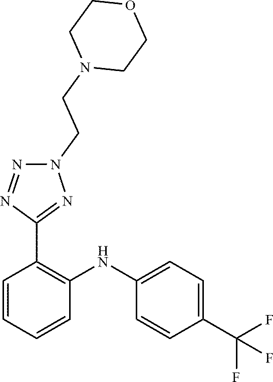 [SMILES: FC(F)(F)c1ccc(Nc2ccccc2-c2nnn(CCN3CCOCC3)n2)cc1] |
| 69 | 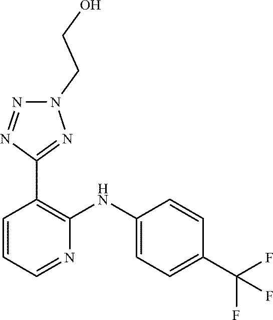 [SMILES: OCCn1nnc(-c2cccnc2Nc2ccc(C(F)(F)F)cc2)n1] |
| 70 | 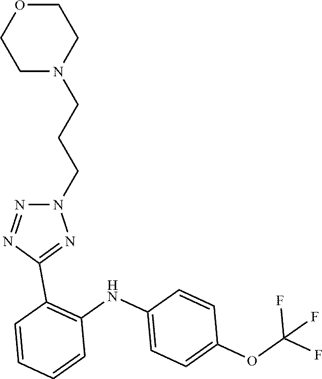 [SMILES: FC(F)(F)Oc1ccc(Nc2ccccc2-c2nnn(CCCN3CCOCC3)n2)cc1] |
| 71 | 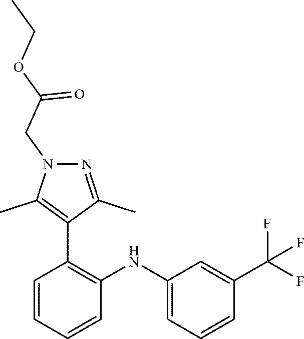 [SMILES: CCOC(=O)Cn1nc(C)c(-c2ccccc2Nc2cccc(C(F)(F)F)c2)c1C] |

| | |
|---|---|
| 72 | 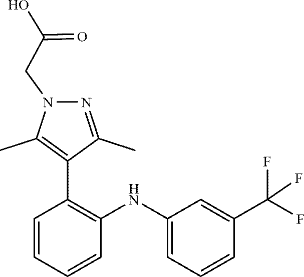 [SMILES: Cc1nn(CC(=O)O)c(C)c1-c1ccccc1Nc1cccc(C(F)(F)F)c1] |
| 73 | 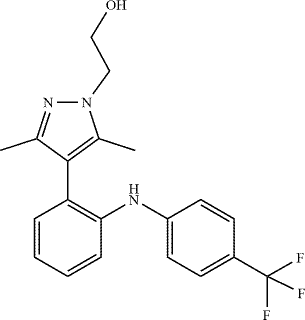 [SMILES: Cc1nn(CCO)c(C)c1-c1ccccc1Nc1ccc(C(F)(F)F)cc1] |
| 74 | 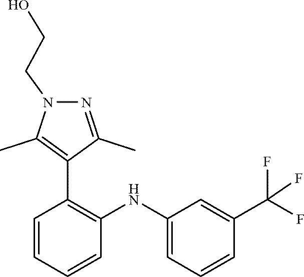 [SMILES: Cc1nn(CCO)c(C)c1-c1ccccc1Nc1cccc(C(F)(F)F)c1] |
| 75 | 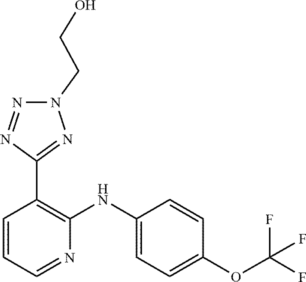 [SMILES: OCCn1nnc(-c2cccnc2Nc2ccc(OC(F)(F)F)cc2)n1] |
| 76 | 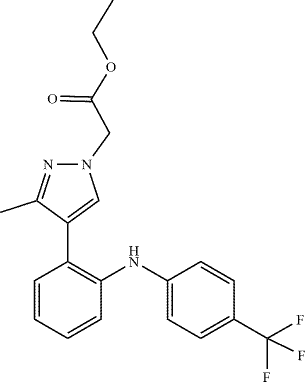 [SMILES: CCOC(=O)Cn1cc(-c2ccccc2Nc2ccc(C(F)(F)F)cc2)c(C)n1] |

| | |
|---|---|
| 77 | 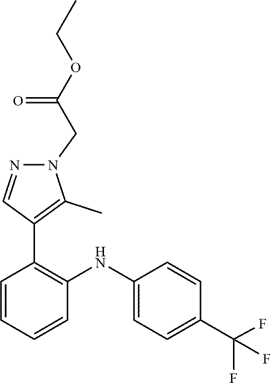 [SMILES: CCOC(=O)Cn1ncc(-c2ccccc2Nc2ccc(C(F)(F)F)cc2)c1C] |
| 78 | 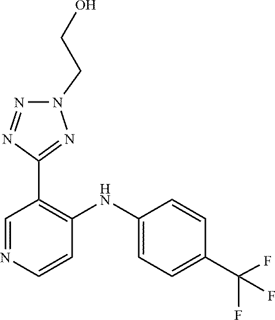 [SMILES: OCCn1nnc(-c2cnccc2Nc2ccc(C(F)(F)F)cc2)n1] |
| 79 | 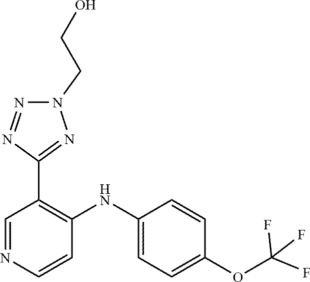 [SMILES: OCCn1nnc(-c2cnccc2Nc2ccc(OC(F)(F)F)cc2)n1] |
| 80 | 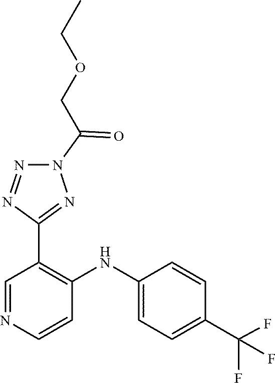 [SMILES: CCOCC(=O)n1nnc(-c2cnccc2Nc2ccc(C(F)(F)F)cc2)n1] |
| 81 | 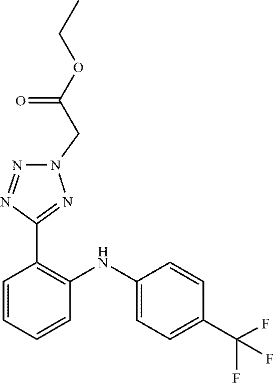 [SMILES: CCOC(=O)Cn1nnc(-c2ccccc2Nc2ccc(C(F)(F)F)cc2)n1] |

| | |
|---|---|
| 82 | 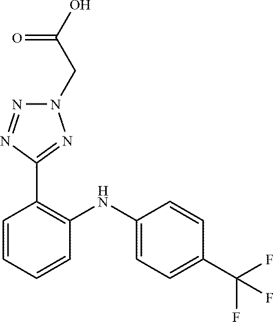 [SMILES: O=C(O)Cn1nnc(-c2ccccc2Nc2ccc(C(F)(F)F)cc2)n1] |
| 83 | 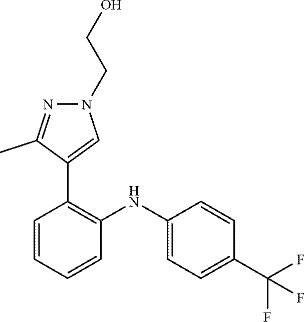 [SMILES: Cc1nn(CCO)cc1-c1ccccc1Nc1ccc(C(F)(F)F)cc1] |
| 84 | 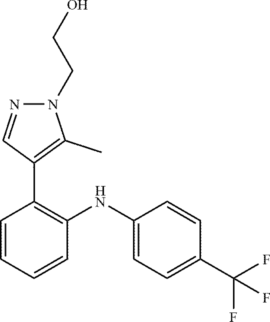 [SMILES: Cc1c(-c2ccccc2Nc2ccc(C(F)(F)F)cc2)cnn1CCO] |
| 85 | 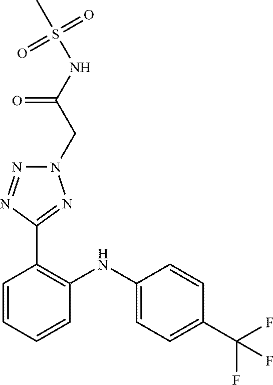 [SMILES: CS(=O)(=O)NC(=O)Cn1nnc(-c2ccccc2Nc2ccc(C(F)(F)F)cc2)n1] |
| 86 | 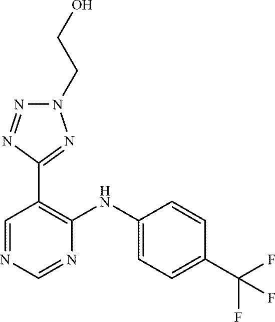 [SMILES: OCCn1nnc(-c2cncnc2Nc2ccc(C(F)(F)F)cc2)n1] |

| | |
|---|---|
| 87 | 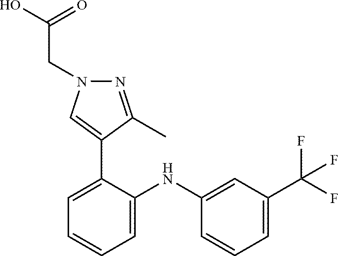 [SMILES: Cc1nn(CC(=O)O)cc1-c1ccccc1Nc1cccc(C(F)(F)F)c1] |
| 88 | 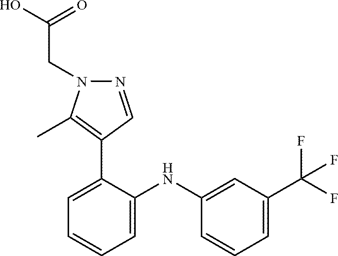 [SMILES: Cc1c(-c2ccccc2Nc2cccc(C(F)(F)F)c2)cnn1CC(=O)O] |
| 89 | 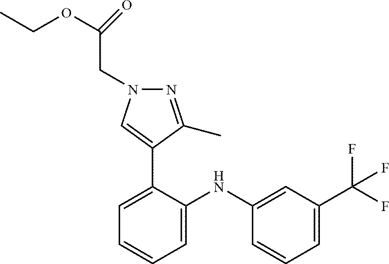 [SMILES: CCOC(=O)Cn1cc(-c2ccccc2Nc2cccc(C(F)(F)F)c2)c(C)n1] |
| 90 | 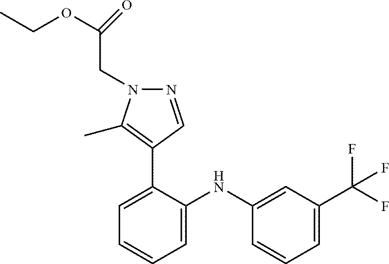 [SMILES: CCOC(=O)Cn1ncc(-c2ccccc2Nc2cccc(C(F)(F)F)c2)c1C] |
| 91 | 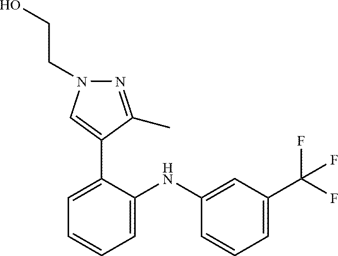 [SMILES: Cc1nn(CCO)cc1-c1ccccc1Nc1cccc(C(F)(F)F)c1] |
| 92 | 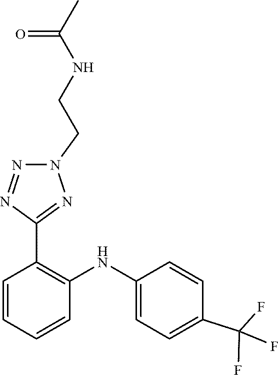 [SMILES: CC(=O)NCCn1nnc(-c2ccccc2Nc2ccc(C(F)(F)F)cc2)n1] |

| | |
|---|---|
| 93 | 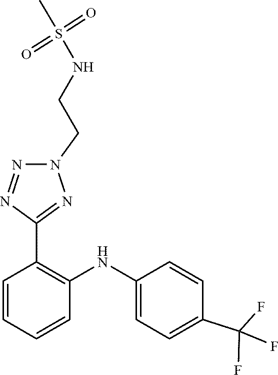 [SMILES: CS(=O)(=O)NCCn1nnc(-c2ccccc2Nc2ccc(C(F)(F)F)cc2)n1] |
| 94 | 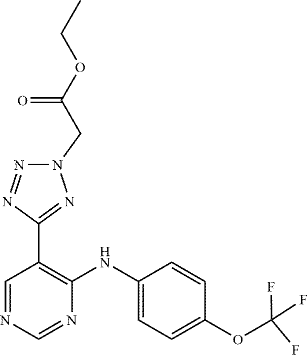 [SMILES: CCOC(=O)Cn1nnc(-c2cncnc2Nc2ccc(OC(F)(F)F)cc2)n1] |
| 95 | 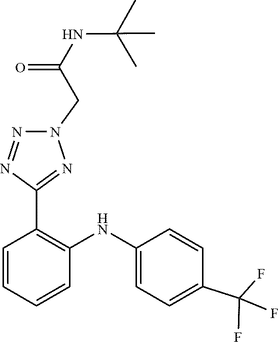 [SMILES: CC(C)(C)NC(=O)Cn1nnc(-c2ccccc2Nc2ccc(C(F)(F)F)cc2)n1] |
| 96 | 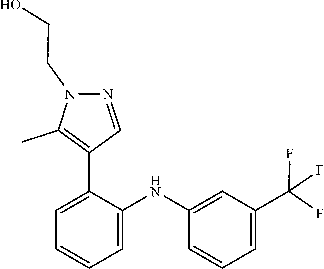 [SMILES: Cc1c(-c2ccccc2Nc2cccc(C(F)(F)F)c2)cnn1CCO] |
| 97 | 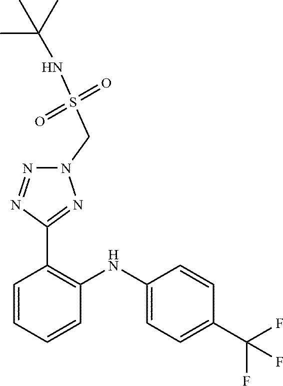 [SMILES: CC(C)(C)NS(=O)(=O)Cn1nnc(-c2ccccc2Nc2ccc(C(F)(F)F)cc2)n1] |

| | |
|---|---|
| 98 | 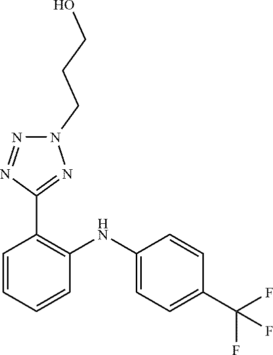 [SMILES: OCCCn1nnc(-c2ccccc2Nc2ccc(C(F)(F)F)cc2)n1] |
| 99 | 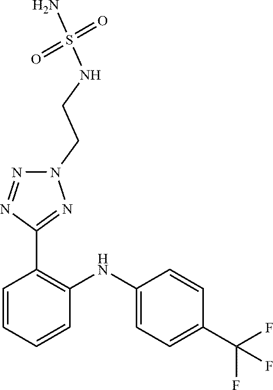 [SMILES: NS(=O)(=O)NCCn1nnc(-c2ccccc2Nc2ccc(C(F)(F)F)cc2)n1] |
| 100 | 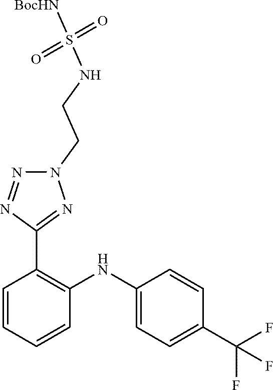 [SMILES: CC(C)(C)OC(=O)NS(=O)(=O)NCCn1nnc(-c2ccccc2Nc2ccc(C(F)(F)F)cc2)n1] |
| 101 | 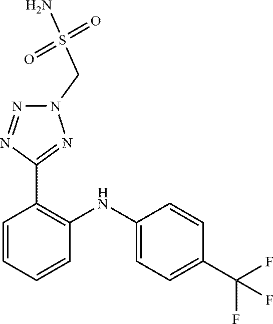 [SMILES: NS(=O)(=O)Cn1nnc(-c2ccccc2Nc2ccc(C(F)(F)F)cc2)n1] |
| 102 | 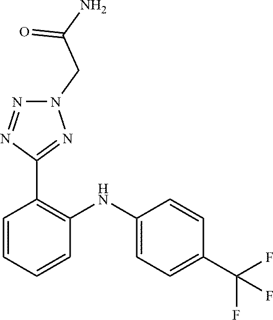 [SMILES: NC(=O)Cn1nnc(-c2ccccc2Nc2ccc(C(F)(F)F)cc2)n1] |

| | |
|---|---|
| 103 | 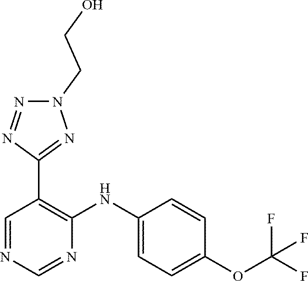 [SMILES: OCCn1nnc(-c2cncnc2Nc2ccc(OC(F)(F)F)cc2)n1] |
| 104 | 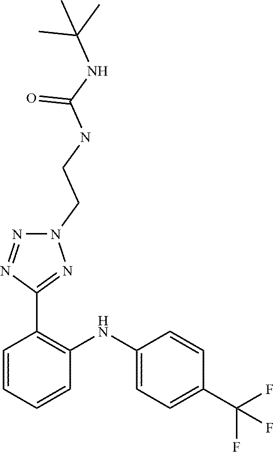 [SMILES: CC(C)(C)NC(=O)NCCn1nnc(-c2ccccc2Nc2ccc(C(F)(F)F)cc2)n1] |
| 105 | 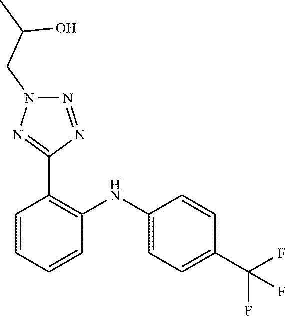 [SMILES: CC(O)Cn1nnc(-c2ccccc2Nc2ccc(C(F)(F)F)cc2)n1] |
| 106 | 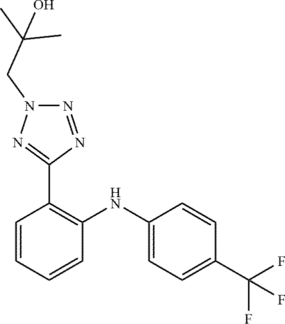 [SMILES: CC(C)(O)Cn1nnc(-c2ccccc2Nc2ccc(C(F)(F)F)cc2)n1] |
| 107 | 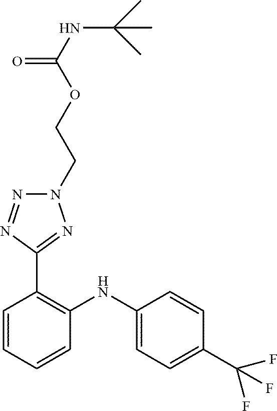 [SMILES: CC(C)(C)NC(=O)OCCn1nnc(-c2ccccc2Nc2ccc(C(F)(F)F)cc2)n1] |

| | |
|---|---|
| 108 | 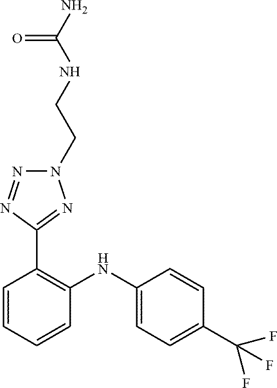 [SMILES: NC(=O)NCCn1nnc(-c2ccccc2Nc2ccc(C(F)(F)F)cc2)n1] |
| 109 | 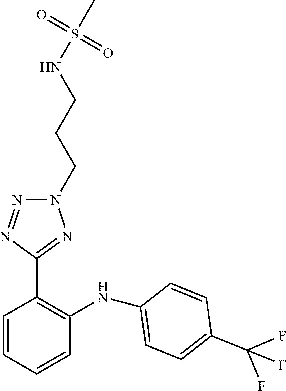 [SMILES: CS(=O)(=O)NCCCn1nnc(-c2ccccc2Nc2ccc(C(F)(F)F)cc2)n1] |
| 110 | 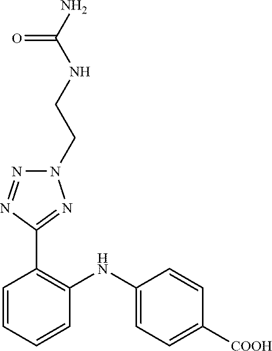 [SMILES: NC(=O)NCCn1nnc(-c2ccccc2Nc2ccc(C(=O)O)cc2)n1] |
| 111 | 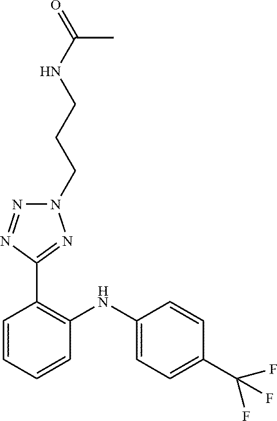 [SMILES: CC(=O)NCCCn1nnc(-c2ccccc2Nc2ccc(C(F)(F)F)cc2)n1] |

| | |
|---|---|
| 112 | 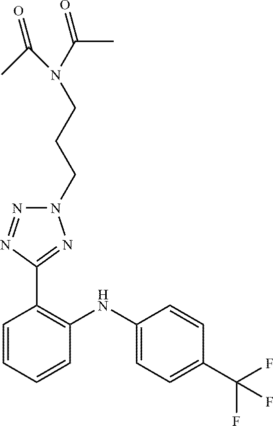 [SMILES: CC(=O)N(CCCn1nnc(-c2ccccc2Nc2ccc(C(F)(F)F)cc2)n1)C(C)=O] |
| 113 | 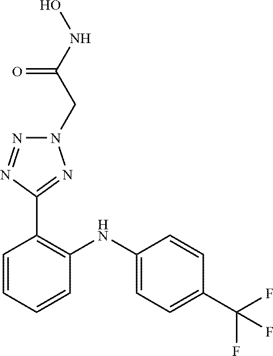 [SMILES: O=C(Cn1nnc(-c2ccccc2Nc2ccc(C(F)(F)F)cc2)n1)NO] |
| 114 | 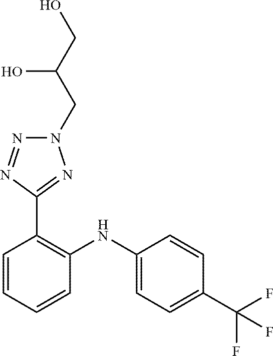 [SMILES: OCC(O)Cn1nnc(-c2ccccc2Nc2ccc(C(F)(F)F)cc2)n1] |
| 115 | 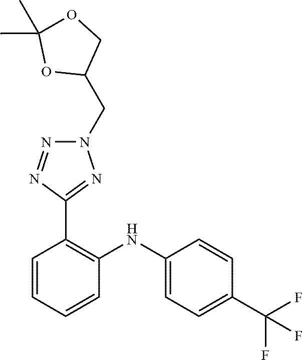 [SMILES: CC1(C)OCC(Cn2nnc(-c3ccccc3Nc3ccc(C(F)(F)F)cc3)n2)O1] |
| 116 | 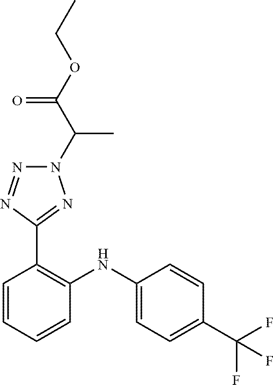 [SMILES: CCOC(=O)C(C)n1nnc(-c2ccccc2Nc2ccc(C(F)(F)F)cc2)n1] |

| | |
|---|---|
| 117 | 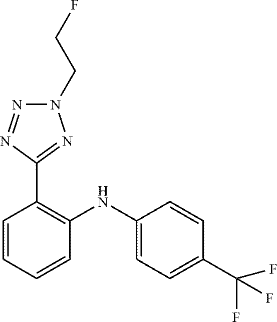 [SMILES: FCCn1nnc(-c2ccccc2Nc2ccc(C(F)(F)F)cc2)n1] |
| 118 | 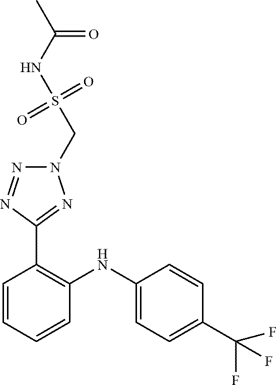 [SMILES: CC(=O)NS(=O)(=O)Cn1nnc(-c2ccccc2Nc2ccc(C(F)(F)F)cc2)n1] |
| 119 | 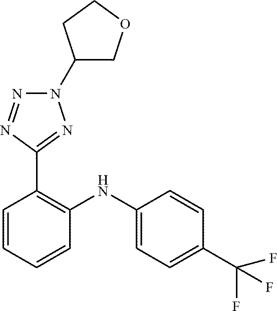 [SMILES: FC(F)(F)c1ccc(Nc2ccccc2-c2nnn(C3CCOC3)n2)cc1] |
| 120 | 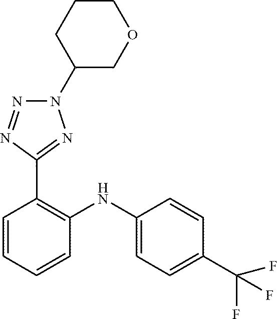 [SMILES: FC(F)(F)c1ccc(Nc2ccccc2-c2nnn(C3CCCOC3)n2)cc1] |
| 121 | 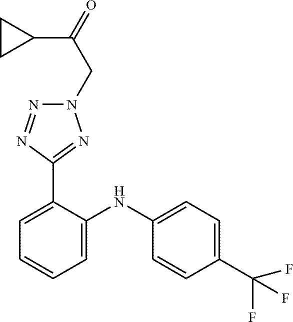 [SMILES: O=C(Cn1nnc(-c2ccccc2Nc2ccc(C(F)(F)F)cc2)n1)C1CC1] |

| | |
|---|---|
| 122 | 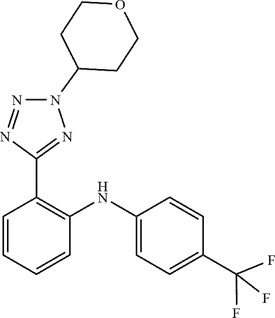 [SMILES: FC(F)(F)c1ccc(Nc2ccccc2-c2nnn(C3CCOCC3)n2)cc1] |
| 123 | 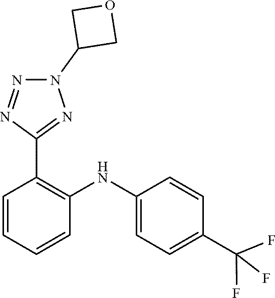 [SMILES: FC(F)(F)c1ccc(Nc2ccccc2-c2nnn(C3COC3)n2)cc1] |
| 124 | 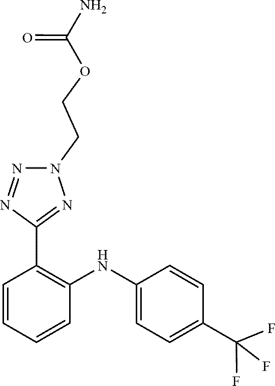 [SMILES: NC(=O)OCCn1nnc(-c2ccccc2Nc2ccc(C(F)(F)F)cc2)n1] |
| 125 | 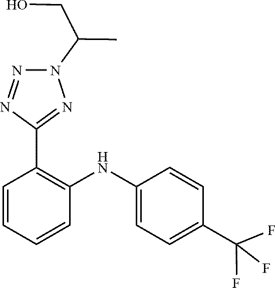 [SMILES: CC(CO)n1nnc(-c2ccccc2Nc2ccc(C(F)(F)F)cc2)n1] |
| 126 | 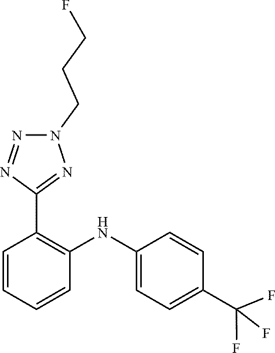 [SMILES: FCCCn1nnc(-c2ccccc2Nc2ccc(C(F)(F)F)cc2)n1] |

| | |
|---|---|
| 127 | 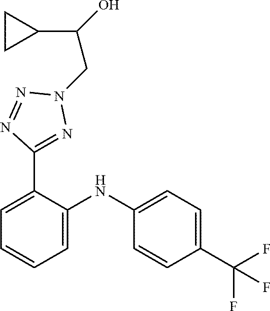 [SMILES: OC(Cn1nnc(-c2ccccc2Nc2ccc(C(F)(F)F)cc2)n1)C1CC1] |
| 128 | 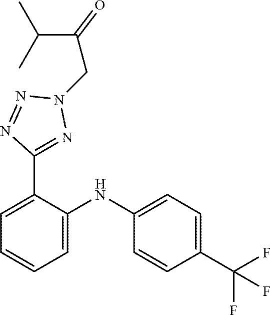 [SMILES: CC(C)C(=O)Cn1nnc(-c2ccccc2Nc2ccc(C(F)(F)F)cc2)n1] |
| 129 | 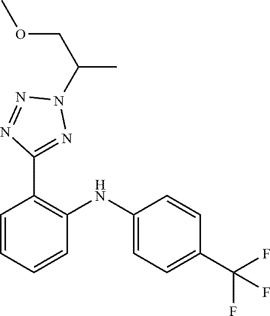 [SMILES: COCC(C)n1nnc(-c2ccccc2Nc2ccc(C(F)(F)F)cc2)n1] |
| 130 | 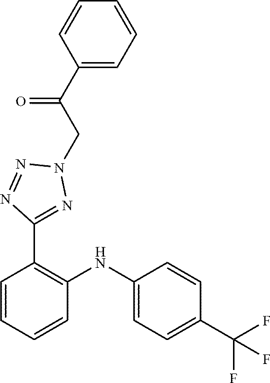 [SMILES: O=C(Cn1nnc(-c2ccccc2Nc2ccc(C(F)(F)F)cc2)n1)c1ccccc1] |
| 131 | 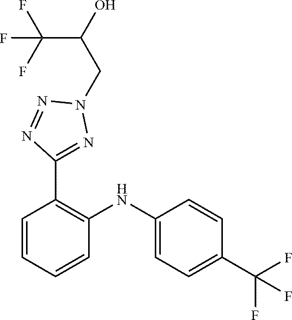 [SMILES: OC(Cn1nnc(-c2ccccc2Nc2ccc(C(F)(F)F)cc2)n1)C(F)(F)F] |

| | |
|---|---|
| 132 | 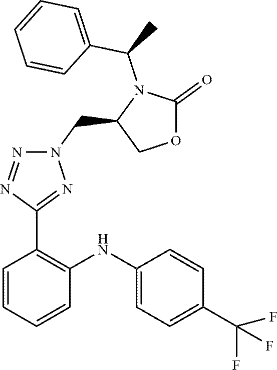 [SMILES: C[C@H](c1ccccc1)N1C(=O)OC[C@H]1Cn1nnc(-c2ccccc2Nc2ccc(C(F)(F)F)cc2)n1] |
| 133 | 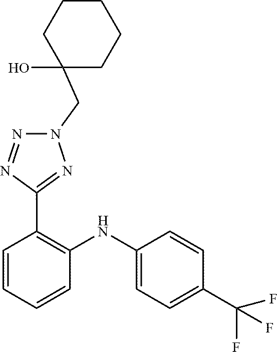 [SMILES: OC1(Cn2nnc(-c3ccccc3Nc3ccc(C(F)(F)F)cc3)n2)CCCCC1] |
| 134 | 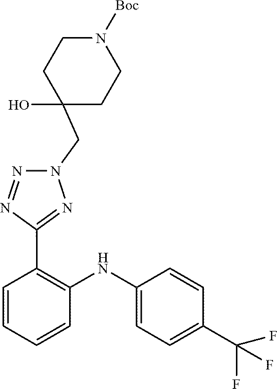 [SMILES: CC(C)(C)OC(=O)N1CCC(O)(Cn2nnc(-c3ccccc3Nc3ccc(C(F)(F)F)cc3)n2)CC1] |
| 135 | 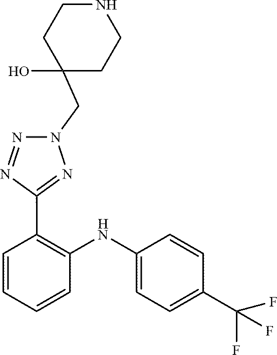 [SMILES: OC1(Cn2nnc(-c3ccccc3Nc3ccc(C(F)(F)F)cc3)n2)CCNCC1] |
| 136 | 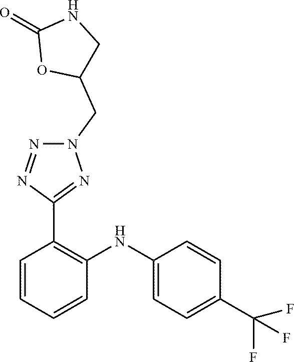 [SMILES: O=C1NCC(Cn2nnc(-c3ccccc3Nc3ccc(C(F)(F)F)cc3)n2)O1] |

| | |
|---|---|
| 137 | 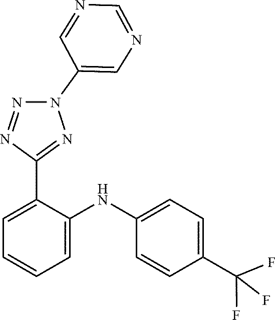 [SMILES: FC(F)(F)c1ccc(Nc2ccccc2-c2nnn(-c3cncnc3)n2)cc1] |
| 138 | 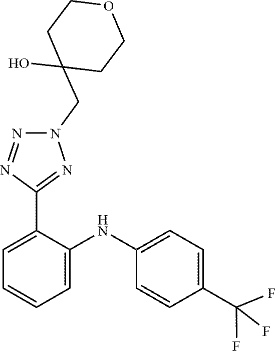 [SMILES: OC1(Cn2nnc(-c3ccccc3Nc3ccc(C(F)(F)F)cc3)n2)CCOCC1] |
| 139 | 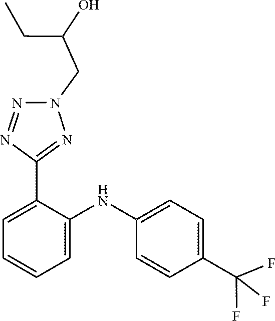 [SMILES: CCC(O)Cn1nnc(-c2ccccc2Nc2ccc(C(F)(F)F)cc2)n1] |
| 140 | 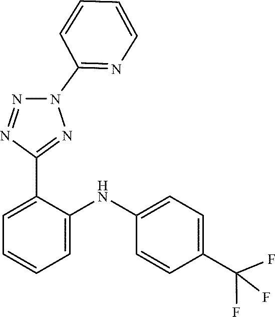 [SMILES: FC(F)(F)c1ccc(Nc2ccccc2-c2nnn(-c3ccccn3)n2)cc1] |
| 141 | 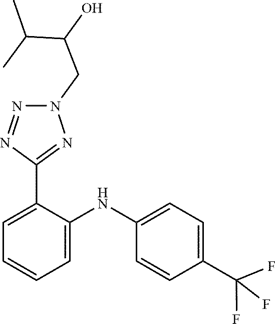 [SMILES: CC(C)C(O)Cn1nnc(-c2ccccc2Nc2ccc(C(F)(F)F)cc2)n1] |

| | |
|---|---|
| 142 | 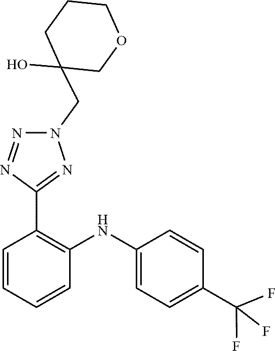 [SMILES: OC1(Cn2nnc(-c3ccccc3Nc3ccc(C(F)(F)F)cc3)n2)CCCOC1] |
| 143 | 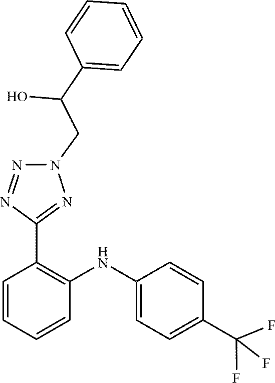 [SMILES: OC(Cn1nnc(-c2ccccc2Nc2ccc(C(F)(F)F)cc2)n1)c1ccccc1] |
| 144 | 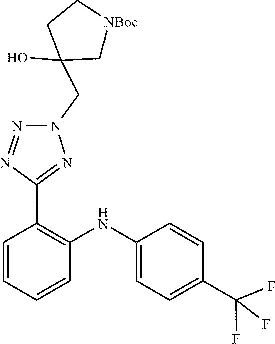 [SMILES: CC(C)(C)OC(=O)N1CCC(O)(Cn2nnc(-c3ccccc3Nc3ccc(C(F)(F)F)cc3)n2)C1] |
| 145 | 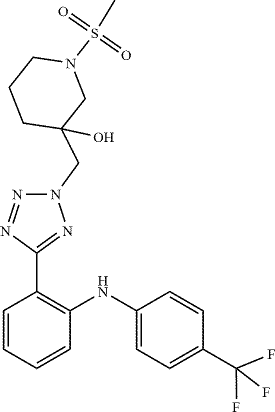 [SMILES: CS(=O)(=O)N1CCCC(O)(Cn2nnc(-c3ccccc3Nc3ccc(C(F)(F)F)cc3)n2)C1] |
| 146 | 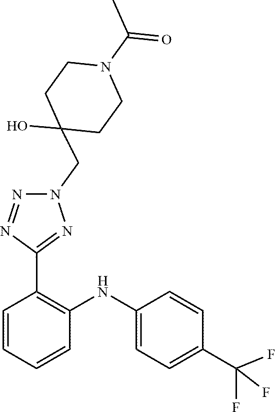 [SMILES: CC(=O)N1CCC(O)(Cn2nnc(-c3ccccc3Nc3ccc(C(F)(F)F)cc3)n2)CC1] |

| | |
|---|---|
| 147 | 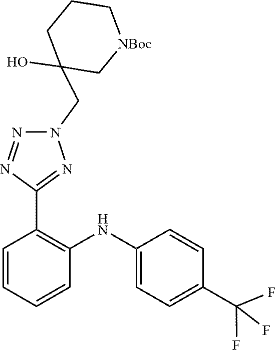 [SMILES: CC(C)(C)OC(=O)N1CCCC(O)(Cn2nnc(-c3ccccc3Nc3ccc(C(F)(F)F)cc3)n2)C1] |
| 148 | 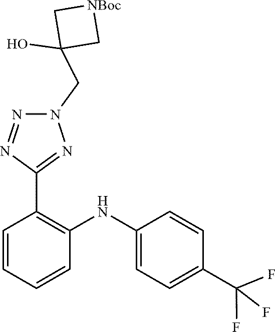 [SMILES: CC(C)(C)OC(=O)N1CC(O)(Cn2nnc(-c3ccccc3Nc3ccc(C(F)(F)F)cc3)n2)C1] |
| 149 | 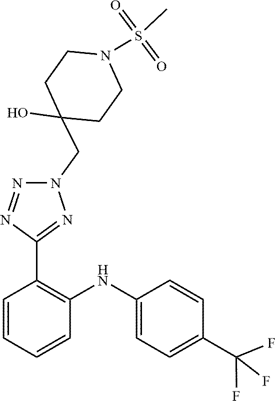 [SMILES: CS(=O)(=O)N1CCC(O)(Cn2nnc(-c3ccccc3Nc3ccc(C(F)(F)F)cc3)n2)CC1] |
| 150 | 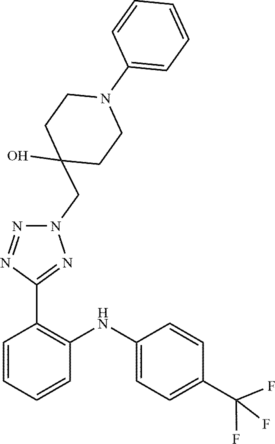 [SMILES: OC1(Cn2nnc(-c3ccccc3Nc3ccc(C(F)(F)F)cc3)n2)CCN(c2ccccc2)CC1] |
| 151 | 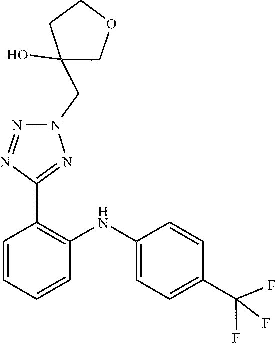 [SMILES: OC1(Cn2nnc(-c3ccccc3Nc3ccc(C(F)(F)F)cc3)n2)CCOC1] |

| | |
|---|---|
| 152 | 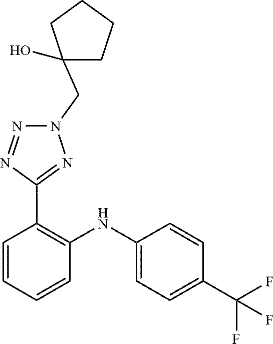 [SMILES: OC1(Cn2nnc(-c3ccccc3Nc3ccc(C(F)(F)F)cc3)n2)CCCC1] |
| 153 | 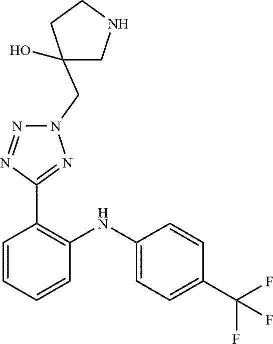 [SMILES: OC1(Cn2nnc(-c3ccccc3Nc3ccc(C(F)(F)F)cc3)n2)CCNC1] |
| 154 | 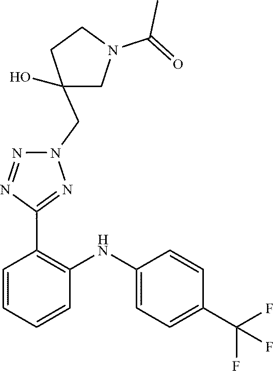 [SMILES: CC(=O)N1CCC(O)(Cn2nnc(-c3ccccc3Nc3ccc(C(F)(F)F)cc3)n2)C1] |
| 155 | 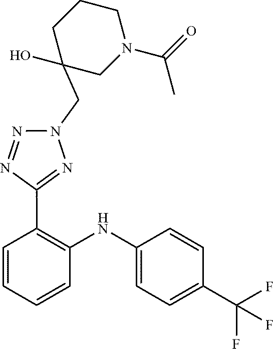 [SMILES: CC(=O)N1CCCC(O)(Cn2nnc(-c3ccccc3Nc3ccc(C(F)(F)F)cc3)n2)C1] |
| 156 | 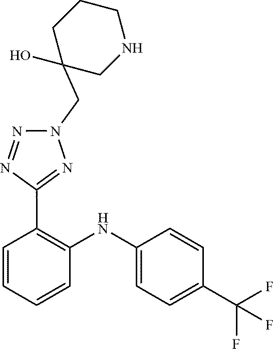 [SMILES: OC1(Cn2nnc(-c3ccccc3Nc3ccc(C(F)(F)F)cc3)n2)CCCNC1] |

| | |
|---|---|
| 157 | 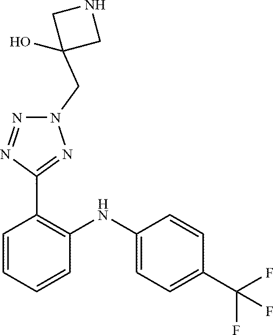 [SMILES: OC1(Cn2nnc(-c3ccccc3Nc3ccc(C(F)(F)F)cc3)n2)CNC1] |
| 158 | 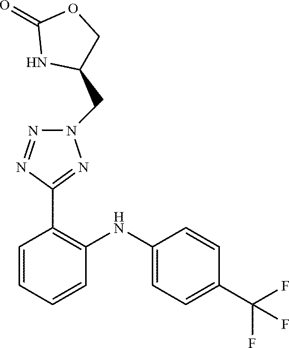 [SMILES: O=C1N[C@H](Cn2nnc(-c3ccccc3Nc3ccc(C(F)(F)F)cc3)n2)CO1] |
| 159 | 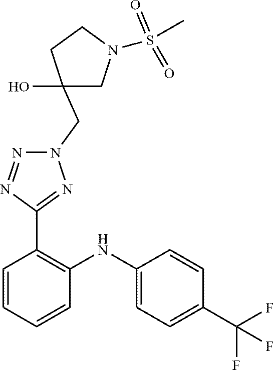 [SMILES: CS(=O)(=O)N1CCC(O)(Cn2nnc(-c3ccccc3Nc3ccc(C(F)(F)F)cc3)n2)C1] |
| 160 | 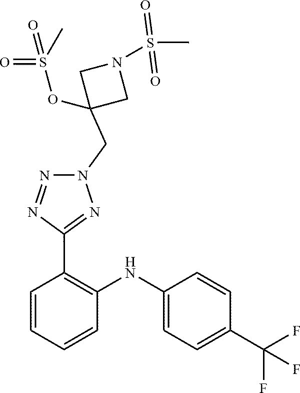 [SMILES: CS(=O)(=O)OC1(Cn2nnc(-c3ccccc3Nc3ccc(C(F)(F)F)cc3)n2)CN(S(C)(=O)=O)C1] |
| 161 | 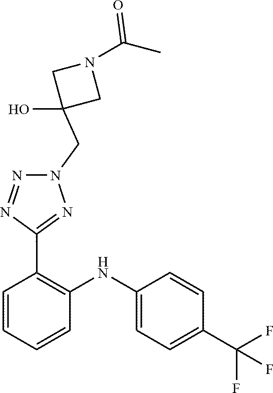 [SMILES: CC(=O)N1CC(O)(Cn2nnc(-c3ccccc3Nc3ccc(C(F)(F)F)cc3)n2)C1] |

| | |
|---|---|
| 162 | 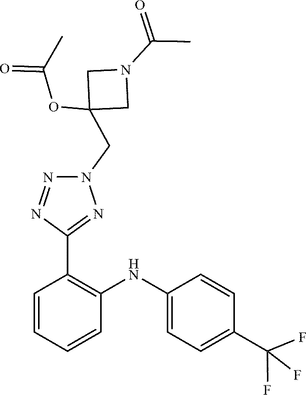 [SMILES: CC(=O)OC1(Cn2nnc(-c3ccccc3Nc3ccc(C(F)(F)F)cc3)n2)CN(C(C)=O)C1] |
| 163 | 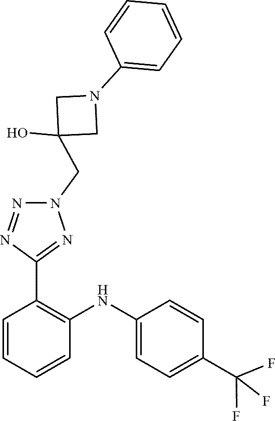 [SMILES: OC1(Cn2nnc(-c3ccccc3Nc3ccc(C(F)(F)F)cc3)n2)CN(c2ccccc2)C1] |
| 164 | 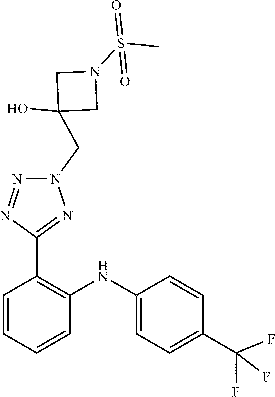 [SMILES: CS(=O)(=O)N1CC(O)(Cn2nnc(-c3ccccc3Nc3ccc(C(F)(F)F)cc3)n2)C1] |
| 165 | 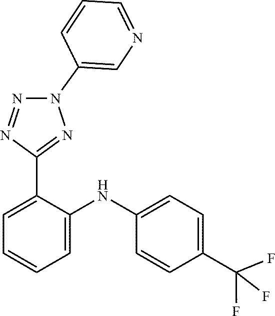 [SMILES: FC(F)(F)c1ccc(Nc2ccccc2-c2nnn(-c3cccnc3)n2)cc1] |
| 166 | 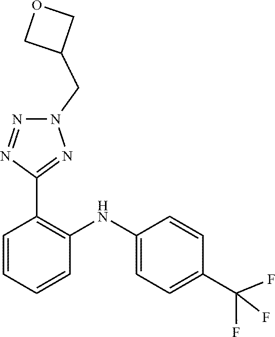 [SMILES: FC(F)(F)c1ccc(Nc2ccccc2-c2nnn(CC3COC3)n2)cc1] |

| | |
|---|---|
| 167 | 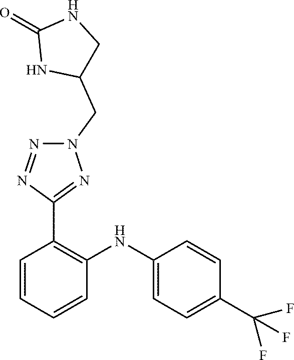 [SMILES: O=C1NCC(Cn2nnc(-c3ccccc3Nc3ccc(C(F)(F)F)cc3)n2)N1] |
| 168 | 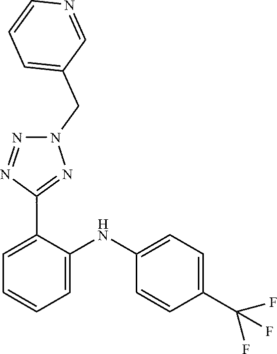 [SMILES: FC(F)(F)c1ccc(Nc2ccccc2-c2nnn(Cc3cccnc3)n2)cc1] |
| 169 | 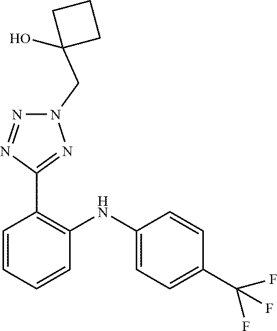 [SMILES: OC1(Cn2nnc(-c3ccccc3Nc3ccc(C(F)(F)F)cc3)n2)CCC1] |
| 170 | 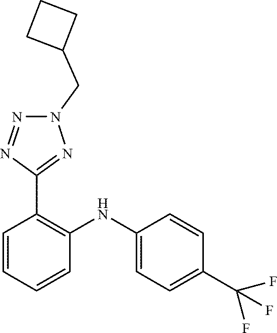 [SMILES: FC(F)(F)c1ccc(Nc2ccccc2-c2nnn(CC3CCC3)n2)cc1] |
| 171 | 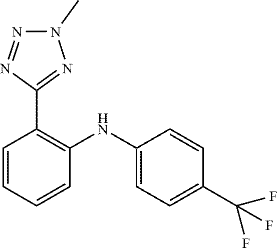 [SMILES: Cn1nnc(-c2ccccc2Nc2ccc(C(F)(F)F)cc2)n1] |

| | |
|---|---|
| 172 | 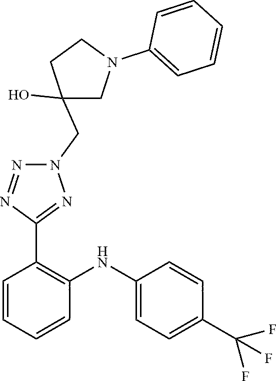 [SMILES: OC1(Cn2nnc(-c3ccccc3Nc3ccc(C(F)(F)F)cc3)n2)CCN(c2ccccc2)C1] |
| 173 | 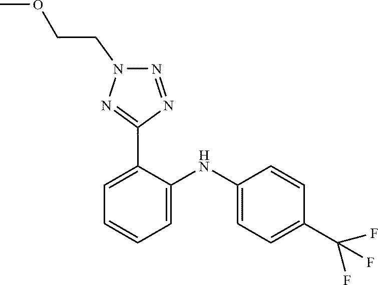 [SMILES: COCCn1nnc(-c2ccccc2Nc2ccc(C(F)(F)F)cc2)n1] |
| 174 | 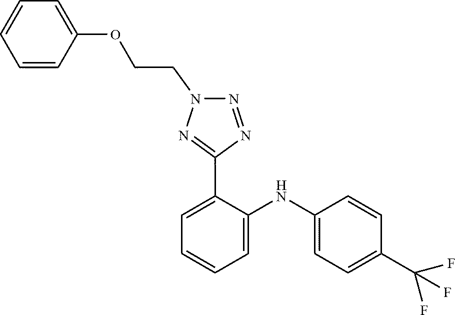 [SMILES: FC(F)(F)c1ccc(Nc2ccccc2-c2nnn(CCOc3ccccc3)n2)cc1] |
| 175 | 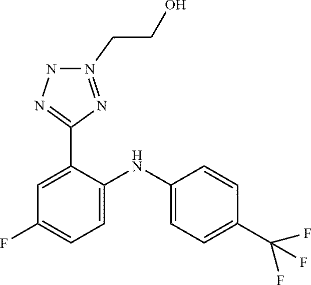 |
| 176 | 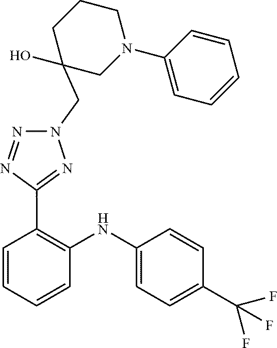 [SMILES: OC1(Cn2nnc(-c3ccccc3Nc3ccc(C(F)(F)F)cc3)n2)CCCN(c2ccccc2)C1] |

| | |
|---|---|
| 177 | 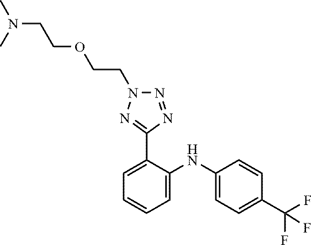 [SMILES: CN(C)CCOCCn1nnc(-c2ccccc2Nc2ccc(C(F)(F)F)cc2)n1] |
| 178 | 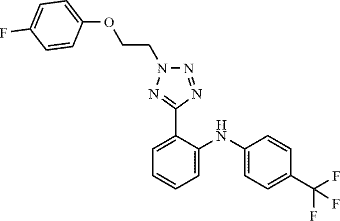 [SMILES: Fc1ccc(OCCn2nnc(-c3ccccc3Nc3ccc(C(F)(F)F)cc3)n2)cc1] |
| 179 | 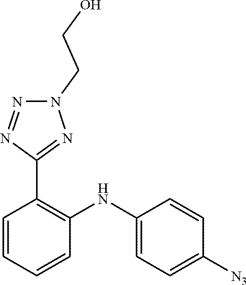 [SMILES: [N-]=[N+]=Nc1ccc(Nc2ccccc2-c2nnn(CCO)n2)cc1] |
| 180 | 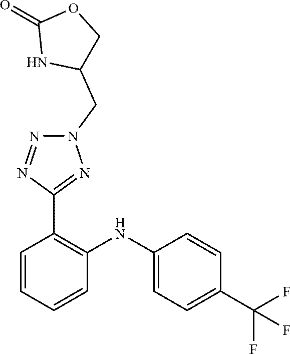 [SMILES: O=C1NC(Cn2nnc(-c3ccccc3Nc3ccc(C(F)(F)F)cc3)n2)CO1] |
| 181 | 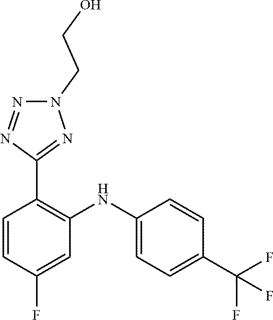 [SMILES: OCCn1nnc(-c2ccc(F)cc2Nc2ccc(C(F)(F)F)cc2)n1] |

| | |
|---|---|
| 182 | 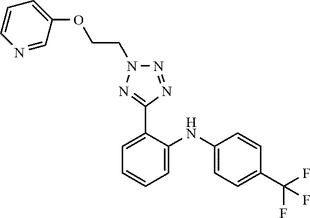 [SMILES: FC(F)(F)c1ccc(Nc2ccccc2-c2nnn(CCOc3cccnc3)n2)cc1] |
| 183 | 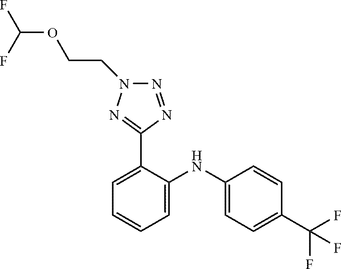 [SMILES: FC(F)OCCn1nnc(-c2ccccc2Nc2ccc(C(F)(F)F)cc2)n1] |
| 184 | 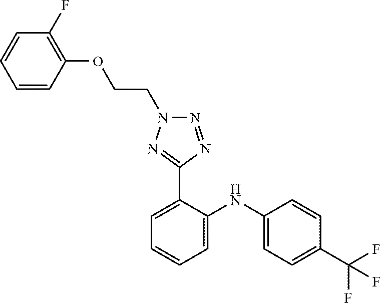 [SMILES: Fc1ccccc1OCCn1nnc(-c2ccccc2Nc2ccc(C(F)(F)F)cc2)n1] |
| 185 | 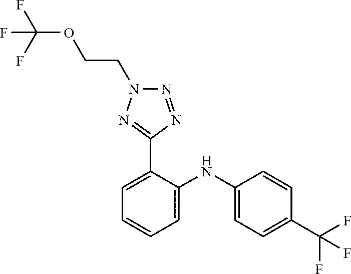 [SMILES: FC(F)(F)OCCn1nnc(-c2ccccc2Nc2ccc(C(F)(F)F)cc2)n1] |
| 186 | 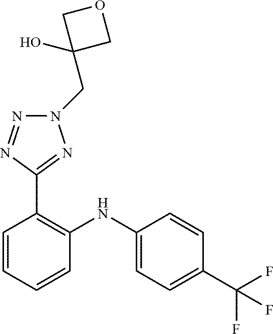 [SMILES: OC1(Cn2nnc(-c3ccccc3Nc3ccc(C(F)(F)F)cc3)n2)COC1] |

| | |
|---|---|
| 187 | 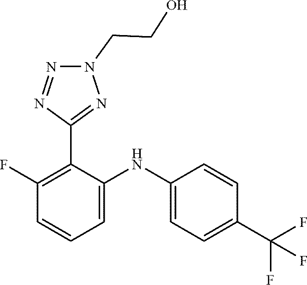 [SMILES: OCCn1nnc(-c2c(F)cccc2Nc2ccc(C(F)(F)F)cc2)n1] |
| 188 | 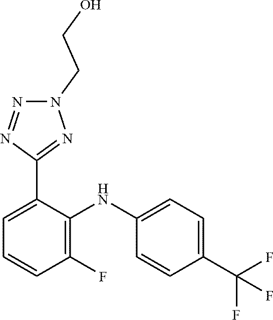 [SMILES: OCCn1nnc(-c2cccc(F)c2Nc2ccc(C(F)(F)F)cc2)n1] |
| 189 | 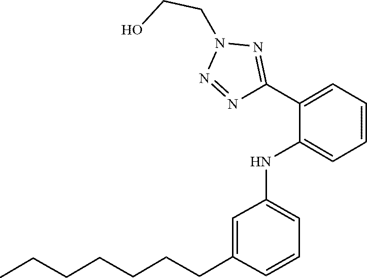 [SMILES: CCCCCCCc1cccc(Nc2ccccc2-c2nnn(CCO)n2)c1] |
| 190 | 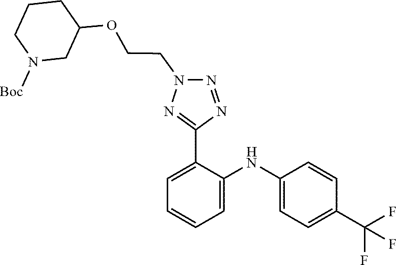 [SMILES: CC(C)(C)OC(=O)N1CCCC(OCCn2nnc(-c3ccccc3Nc3ccc(C(F)(F)F)cc3)n2)C1] |
| 191 | 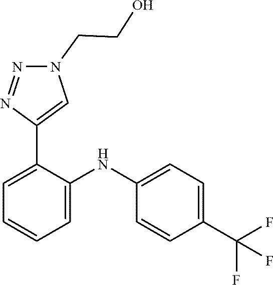 [SMILES: OCCn1cc(-c2ccccc2Nc2ccc(C(F)(F)F)cc2)nn1] |
| 192 | 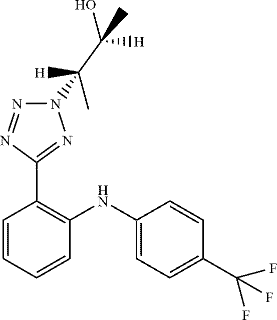 [SMILES: C[C@H](O)[C@H](C)n1nnc(-c2ccccc2Nc2ccc(C(F)(F)F)cc2)n1] |

| | |
|---|---|
| 193 | 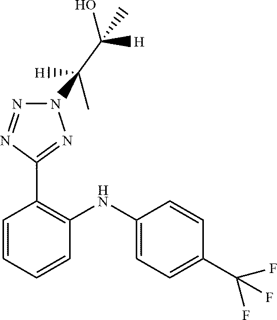 [SMILES: C[C@H]([C@@H](C)O)n1nnc(-c2ccccc2Nc2ccc(C(F)(F)F)cc2)n1] |
| 194 | 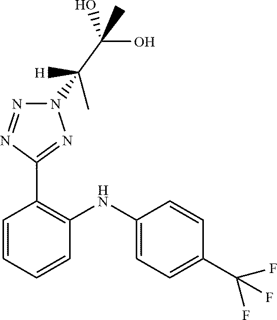 |
| 195 | 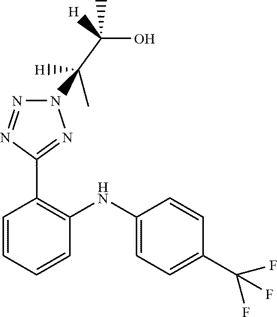 [SMILES: C[C@H](O)[C@@H](C)n1nnc(-c2ccccc2Nc2ccc(C(F)(F)F)cc2)n1] |
| 196 | 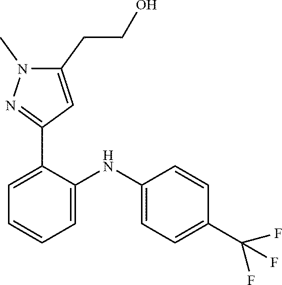 [SMILES: Cn1nc(-c2ccccc2Nc2ccc(C(F)(F)F)cc2)cc1CCO] |
| 197 | 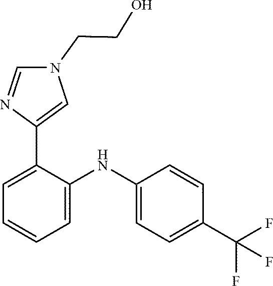 [SMILES: OCCn1cnc(-c2ccccc2Nc2ccc(C(F)(F)F)cc2)c1] |
| 198 | 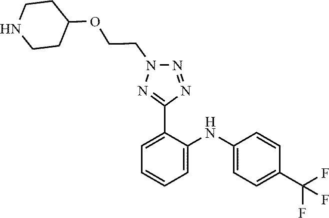 [SMILES: FC(F)(F)c1ccc(Nc2ccccc2-c2nnn(CCOC3CCNCC3)n2)cc1] |

| | |
|---|---|
| 199 | 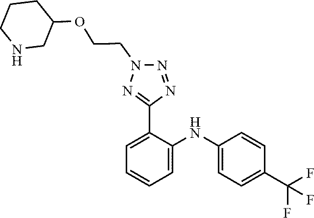 [SMILES: FC(F)(F)c1ccc(Nc2ccccc2-c2nnn(CCOC3CCCNC3)n2)cc1] |
| 200 | 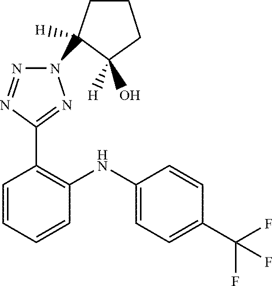 |
| 201 | 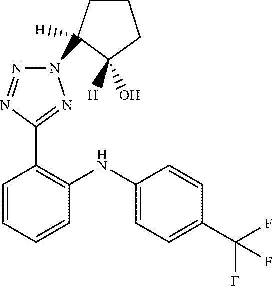 |
| 202 | 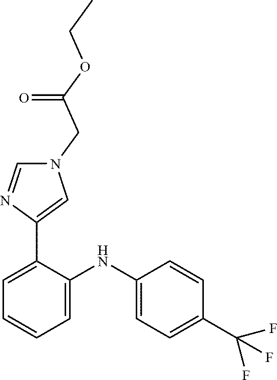 [SMILES: CCOC(=O)Cn1cnc(-c2ccccc2Nc2ccc(C(F)(F)F)cc2)c1] |
| 203 | 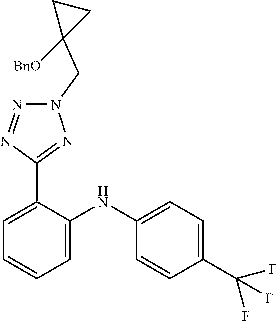 [SMILES: FC(F)(F)c1ccc(Nc2ccccc2-c2nnn(CC3(OCc4ccccc4)CC3)n2)cc1] |

| | |
|---|---|
| 204 | 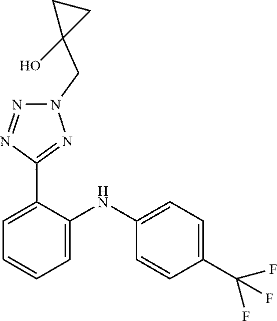 [SMILES: OC1(Cn2nnc(-c3ccccc3Nc3ccc(C(F)(F)F)cc3)n2)CC1] |
| 205 | 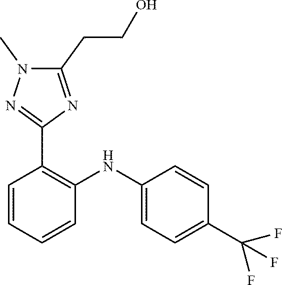 [SMILES: Cn1nc(-c2ccccc2Nc2ccc(C(F)(F)F)cc2)nc1CCO] |
| 206 | 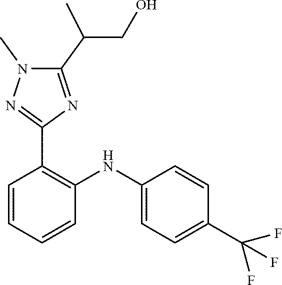 [SMILES: CC(CO)c1nc(-c2ccccc2Nc2ccc(C(F)(F)F)cc2)nn1C] |
| 207 | 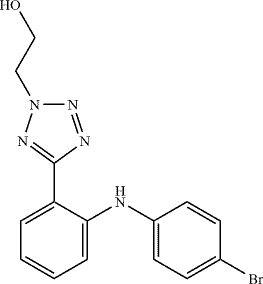 [SMILES: OCCn1nnc(-c2ccccc2Nc2ccc(Br)cc2)n1] |
| 208 | 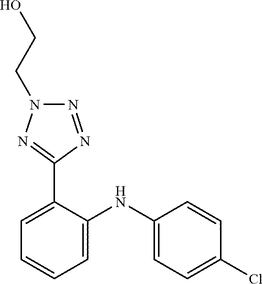 [SMILES: OCCn1nnc(-c2ccccc2Nc2ccc(Cl)cc2)n1] |
| 209 | 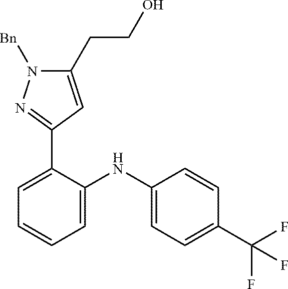 [SMILES: OCCc1cc(-c2ccccc2Nc2ccc(C(F)(F)F)cc2)nn1Cc1ccccc1] |

| | |
|---|---|
| 210 | 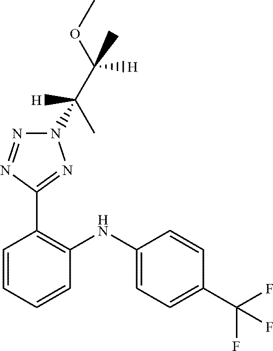 [SMILES: CO[C@@H](C)[C@H](C)n1nnc(-c2ccccc2Nc2ccc(C(F)(F)F)cc2)n1] |
| 211 | 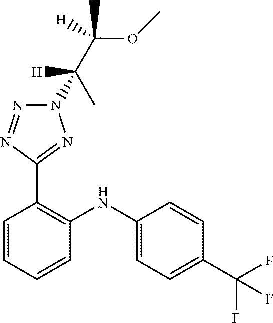 [SMILES: CO[C@H](C)[C@H](C)n1nnc(-c2ccccc2Nc2ccc(C(F)(F)F)cc2)n1] |
| 212 | 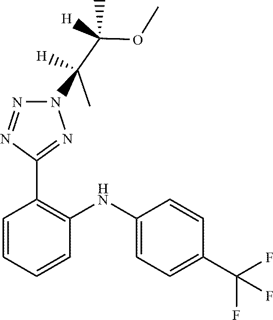 [SMILES: CO[C@@H](C)[C@@H](C)n1nnc(-c2ccccc2Nc2ccc(C(F)(F)F)cc2)n1] |
| 213 | 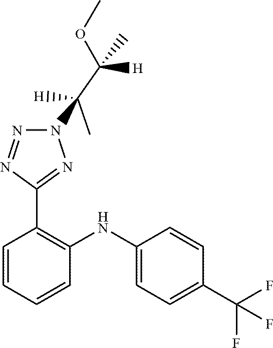 [SMILES: CO[C@H](C)[C@@H](C)n1nnc(-c2ccccc2Nc2ccc(C(F)(F)F)cc2)n1] |
| 214 | 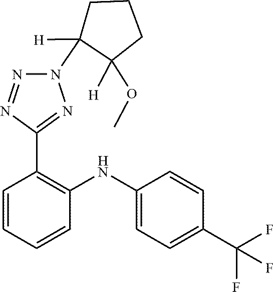 [SMILES: COC1CCCC1n1nnc(-c2ccccc2Nc2ccc(C(F)(F)F)cc2)n1] |

| | |
|---|---|
| 215 | 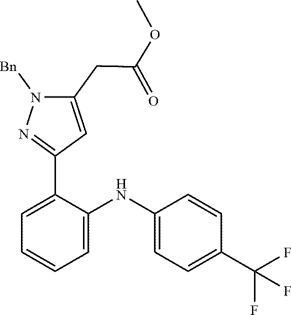 [SMILES: COC(=O)Cc1cc(-c2ccccc2Nc2ccc(C(F)(F)F)cc2)nn1Cc1ccccc1] |
| 216 | 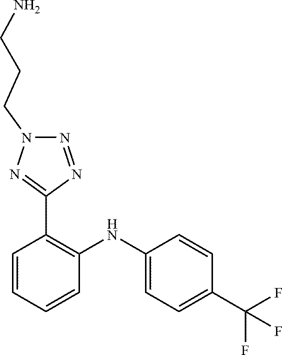 [SMILES: NCCCn1nnc(-c2ccccc2Nc2ccc(C(F)(F)F)cc2)n1] |
| 217 | 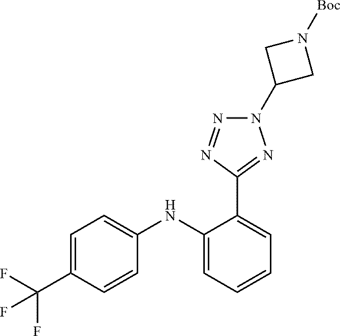 [SMILES: CC(C)(C)OC(=O)N1CC(n2nnc(-c3ccccc3Nc3ccc(C(F)(F)F)cc3)n2)C1] |
| 218 | 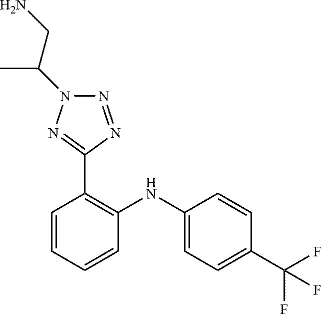 [SMILES: CC(CN)n1nnc(-c2ccccc2Nc2ccc(C(F)(F)F)cc2)n1] |
| 219 | 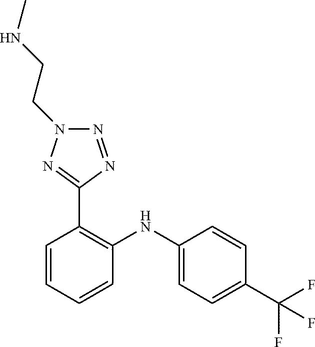 [SMILES: CNCCn1nnc(-c2ccccc2Nc2ccc(C(F)(F)F)cc2)n1] |

| | |
|---|---|
| 220 | 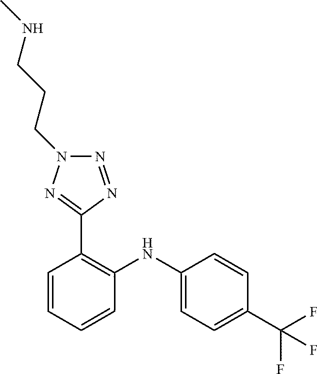 [SMILES: CNCCCn1nnc(-c2ccccc2Nc2ccc(C(F)(F)F)cc2)n1] |
| 221 | 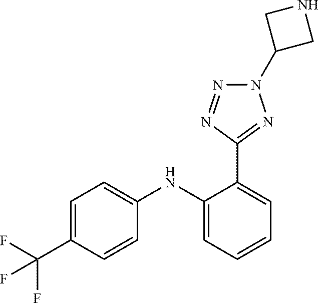 [SMILES: FC(F)(F)c1ccc(Nc2ccccc2-c2nnn(C3CNC3)n2)cc1] |
| 222 | 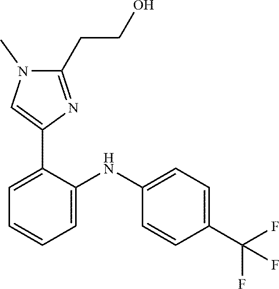 [SMILES: Cn1cc(-c2ccccc2Nc2ccc(C(F)(F)F)cc2)nc1CCO] |
| 223 | 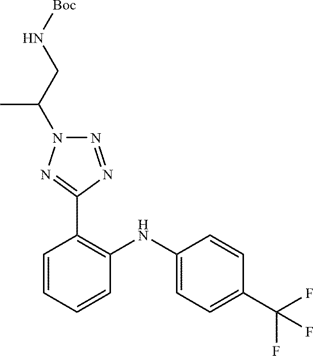 [SMILES: CC(CNC(=O)OC(C)(C)C)n1nnc(-c2ccccc2Nc2ccc(C(F)(F)F)cc2)n1] |
| 224 | 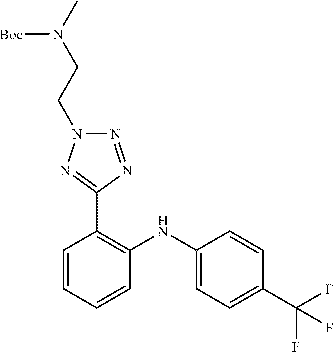 [SMILES: CN(CCn1nnc(-c2ccccc2Nc2ccc(C(F)(F)F)cc2)n1)C(=O)OC(C)(C)C] |

| | |
|---|---|
| 225 | 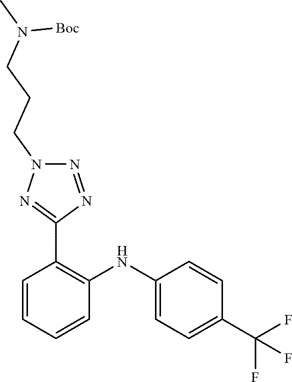 [SMILES: CN(CCCn1nnc(-c2ccccc2Nc2ccc(C(F)(F)F)cc2)n1)C(=O)OC(C)(C)C] |
| 226 | 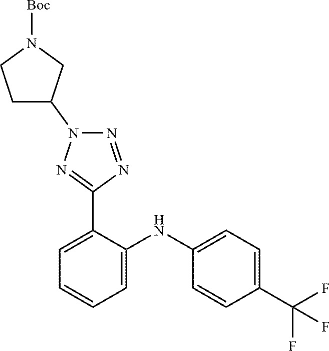 [SMILES: CC(C)(C)OC(=O)N1CCC(n2nnc(-c3ccccc3Nc3ccc(C(F)(F)F)cc3)n2)C1] |
| 227 | 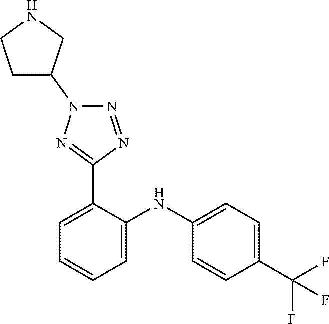 [SMILES: FC(F)(F)c1ccc(Nc2ccccc2-c2nnn(C3CCNC3)n2)cc1] |
| 228 | 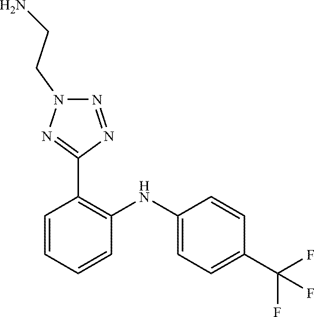 [SMILES: NCCn1nnc(-c2ccccc2Nc2ccc(C(F)(F)F)cc2)n1] |
| 229 | 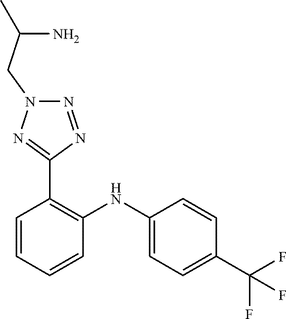 [SMILES: CC(N)Cn1nnc(-c2ccccc2Nc2ccc(C(F)(F)F)cc2)n1] |

| | |
|---|---|
| 230 | 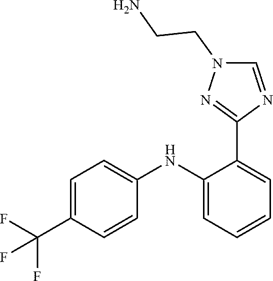 [SMILES: NCCn1cnc(-c2ccccc2Nc2ccc(C(F)(F)F)cc2)n1] |
| 231 | 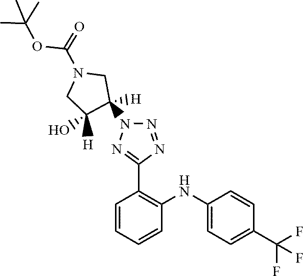 [SMILES: CC(C)(C)OC(=O)N1C[C@@H](O)[C@H](n2nnc(-c3ccccc3Nc3ccc(C(F)(F)F)cc3)n2)C1] |
| 232 | 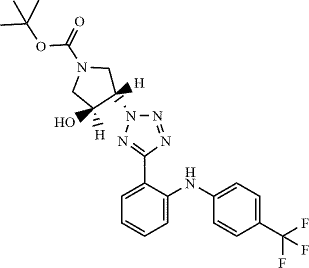 |
| 233 | 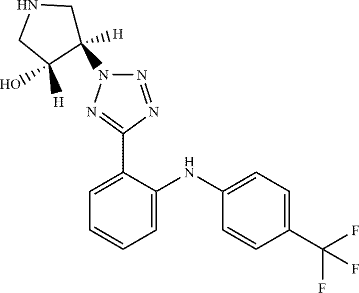 [SMILES: O[C@@H]1CNC[C@H]1n1nnc(-c2ccccc2Nc2ccc(C(F)(F)F)cc2)n1] |
| 234 | 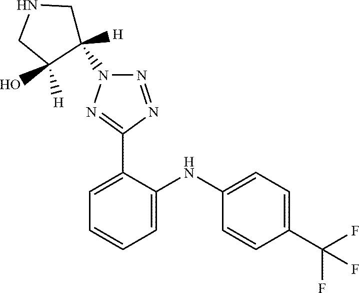 [SMILES: O[C@H]1CNC[C@@H]1n1nnc(-c2ccccc2Nc2ccc(C(F)(F)F)cc2)n1] |

| | |
|---|---|
| 235 | 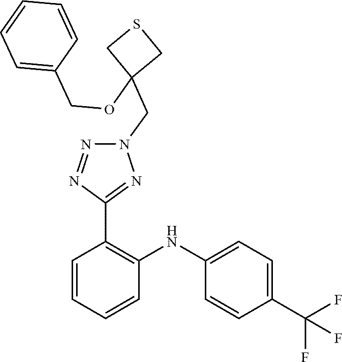 [SMILES: FC(F)(F)c1ccc(Nc2ccccc2-c2nnn(CC3(OCc4ccccc4)CSC3)n2)cc1] |
| 236 | 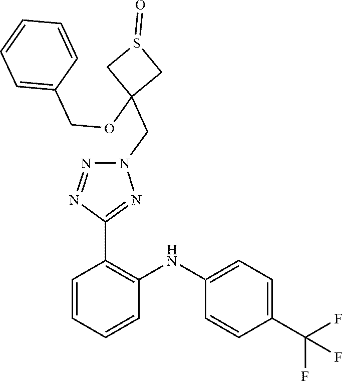 [SMILES: O=S1CC(Cn2nnc(-c3ccccc3Nc3ccc(C(F)(F)F)cc3)n2)(OCc2ccccc2)C1] |
| 237 | 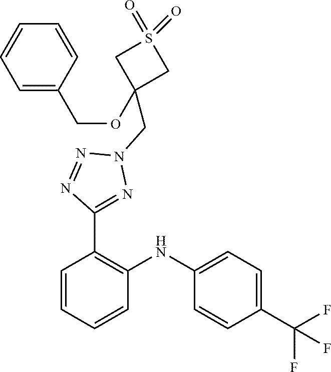 [SMILES: O=S1(=O)CC(Cn2nnc(-c3ccccc3Nc3ccc(C(F)(F)F)cc3)n2)(OCc2ccccc2)C1] |
| 238 | 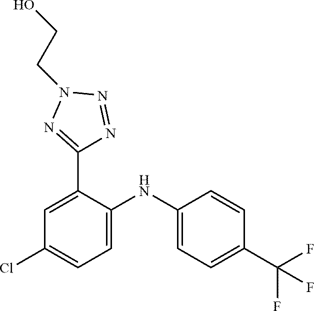 [SMILES: OCCn1nnc(-c2cc(Cl)ccc2Nc2ccc(C(F)(F)F)cc2)n1] |
| 239 | 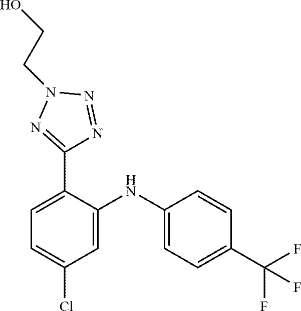 [SMILES: OCCn1nnc(-c2ccc(Cl)cc2Nc2ccc(C(F)(F)F)cc2)n1] |

| | |
|---|---|
| 240 | 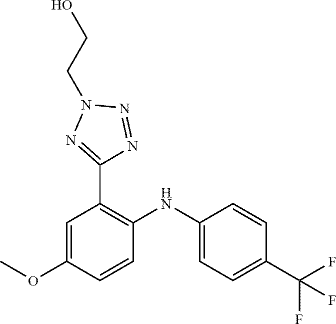 [SMILES: COc1ccc(Nc2ccc(C(F)(F)F)cc2)c(-c2nnn(CCO)n2)c1] |
| 241 | 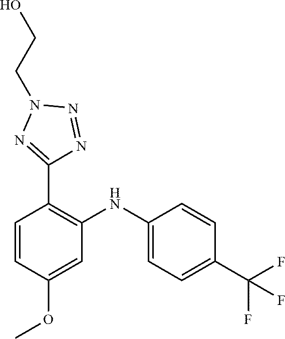 [SMILES: COc1ccc(-c2nnn(CCO)n2)c(Nc2ccc(C(F)(F)F)cc2)c1] |
| 242 | 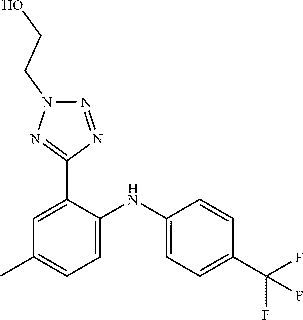 [SMILES: Cc1ccc(Nc2ccc(C(F)(F)F)cc2)c(-c2nnn(CCO)n2)c1] |
| 243 | 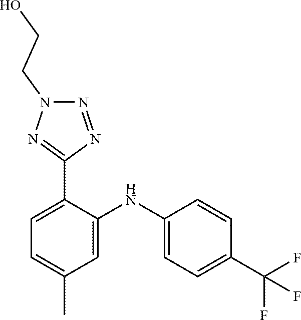 [SMILES: Cc1ccc(-c2nnn(CCO)n2)c(Nc2ccc(C(F)(F)F)cc2)c1] |
| 244 | 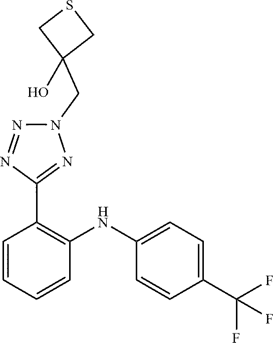 [SMILES: OC1(Cn2nnc(-c3ccccc3Nc3ccc(C(F)(F)F)cc3)n2)CSC1] |

| | |
|---|---|
| 245 | 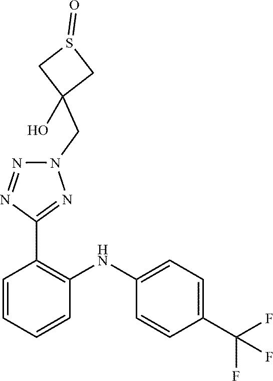 [SMILES: O=S1CC(O)(Cn2nnc(-c3ccccc3Nc3ccc(C(F)(F)F)cc3)n2)C1] |
| 246 | 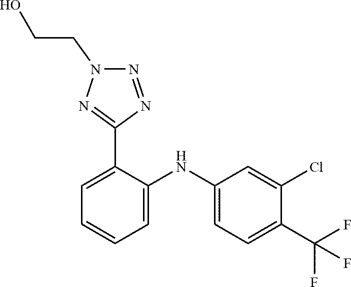 [SMILES: OCCn1nnc(-c2ccccc2Nc2ccc(C(F)(F)F)c(Cl)c2)n1] |
| 247 | 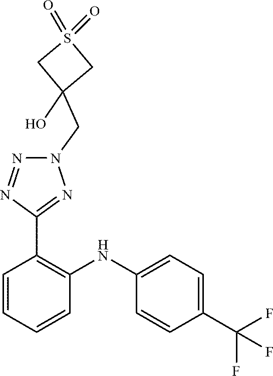 [SMILES: O=S1(=O)CC(O)(Cn2nnc(-c3ccccc3Nc3ccc(C(F)(F)F)cc3)n2)C1] |
| 248 | 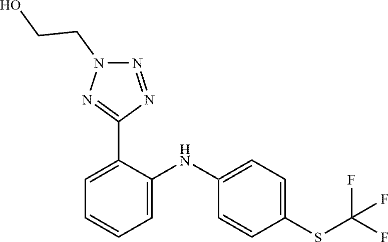 [SMILES: OCCn1nnc(-c2ccccc2Nc2ccc(SC(F)(F)F)cc2)n1] |
| 249 | 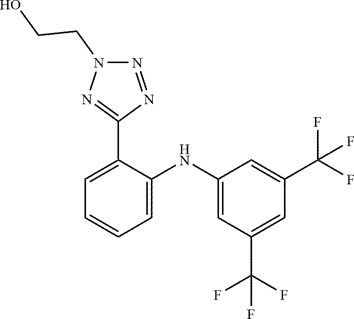 [SMILES: OCCn1nnc(-c2ccccc2Nc2cc(C(F)(F)F)cc(C(F)(F)F)c2)n1] |
| 250 | 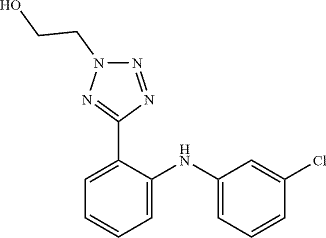 [SMILES: OCCn1nnc(-c2ccccc2Nc2cccc(Cl)c2)n1] |

| | |
|---|---|
| 251 | 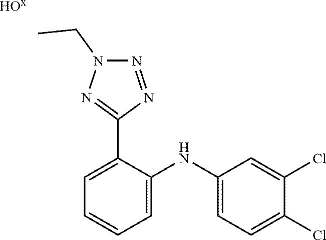 |
| 252 | 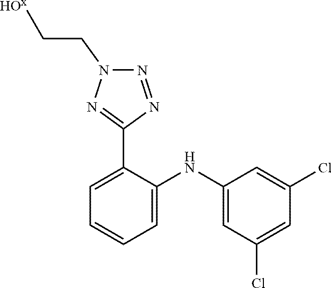 |
| 253 | 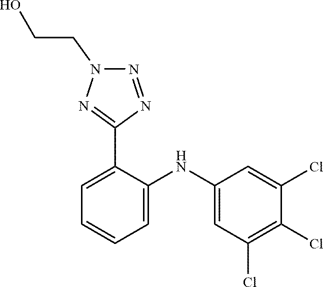 [SMILES: OCCn1nnc(-c2ccccc2Nc2cc(Cl)c(Cl)c(Cl)c2)n1] |
| 254 | 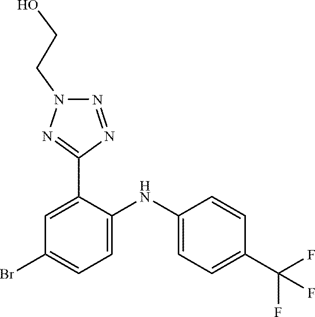 [SMILES: OCCn1nnc(-c2cc(Br)ccc2Nc2ccc(C(F)(F)F)cc2)n1] |
| 255 | 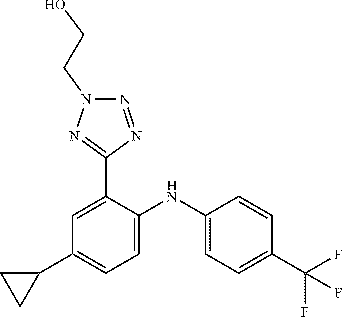 [SMILES: OCCn1nnc(-c2cc(C3CC3)ccc2Nc2ccc(C(F)(F)F)cc2)n1] |
| 256 | 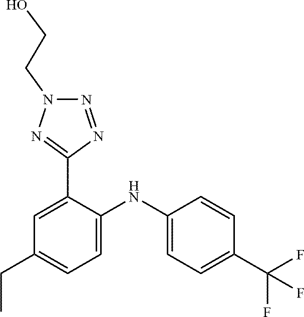 [SMILES: CCc1ccc(Nc2ccc(C(F)(F)F)cc2)c(-c2nnn(CCO)n2)c1] |

| | |
|---|---|
| 257 | 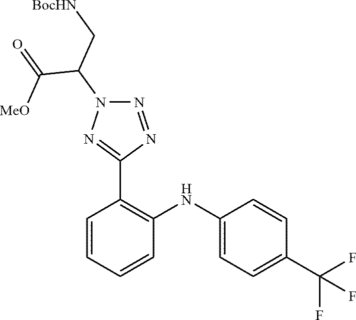 [SMILES: COC(=O)C(CNC(=O)OC(C)(C)C)n1nnc(-c2ccccc2Nc2ccc(C(F)(F)F)cc2)n1] |
| 258 | 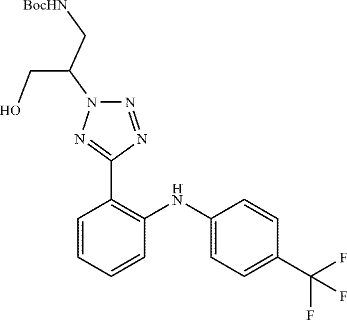 [SMILES: CC(C)(C)OC(=O)NCC(CO)n1nnc(-c2ccccc2Nc2ccc(C(F)(F)F)cc2)n1] |
| 259 | 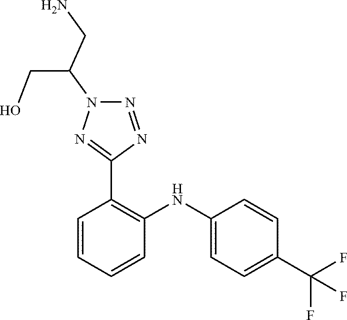 [SMILES: NCC(CO)n1nnc(-c2ccccc2Nc2ccc(C(F)(F)F)cc2)n1] |
| 260 | 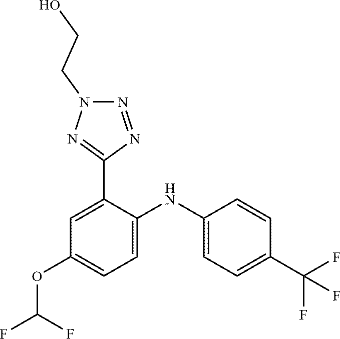 [SMILES: OCCn1nnc(-c2cc(OC(F)F)ccc2Nc2ccc(C(F)(F)F)cc2)n1] |
| 261 | 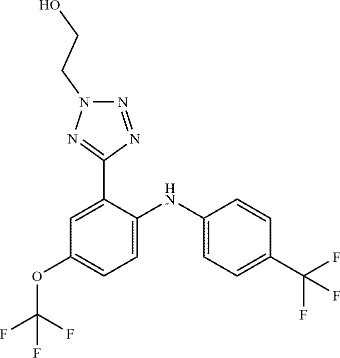 [SMILES: OCCn1nnc(-c2cc(OC(F)(F)F)ccc2Nc2ccc(C(F)(F)F)cc2)n1] |

| | |
|---|---|
| 262 | 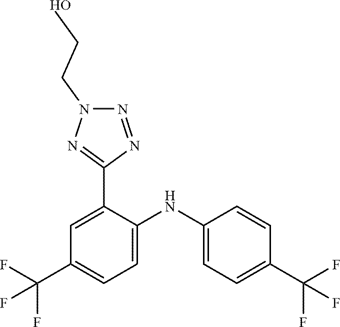 |
| 263 | 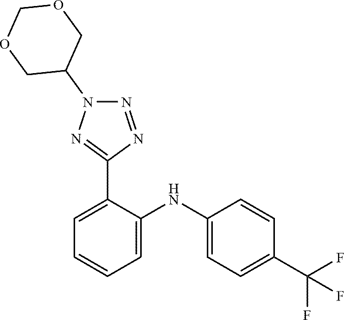 [SMILES: FC(F)(F)c1ccc(Nc2ccccc2-c2nnn(C3COCOC3)n2)cc1] |
| 264 | 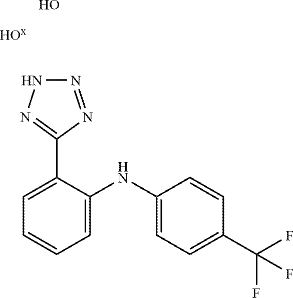 |
| 265 | 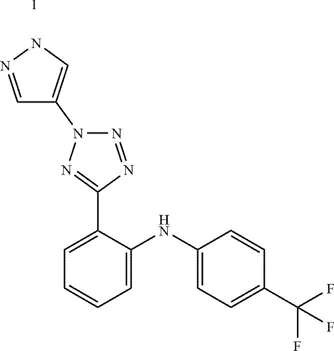 |
| 266 | 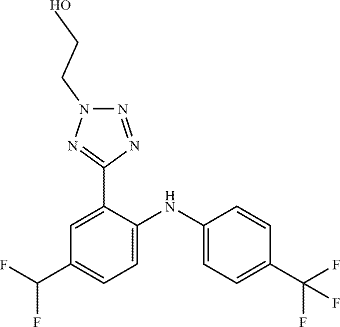 |

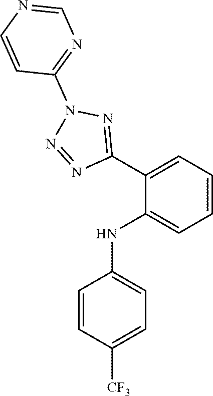 [SMILES: FC(F)(F)c1ccc(Nc2ccccc2-c2nnn(-c3ccncn3)n2)cc1]

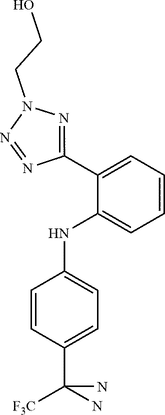 [SMILES: NC(N)(c1ccc(Nc2ccccc2-c2nnn(CCO)n2)cc1)C(F)(F)F]

published Nov. 8, 2018; or PCT Application Publication No. WO 2019/040380 (e.g., the compound of Formula (I-A) is not a compound of Tables 1-2, Examples 1-141 or A1 of WO 2019/040380:

| Compound No. | Structure |
|---|---|
| 1 | 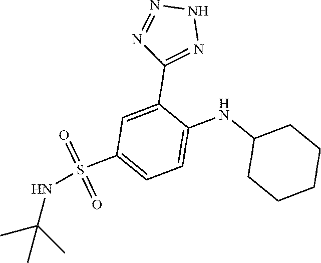 [SMILES: CC(C)(C)NS(=O)(=O)c1ccc(NC2CCCCC2)c(-c2nn[nH]n2)c1] |
| 2 | 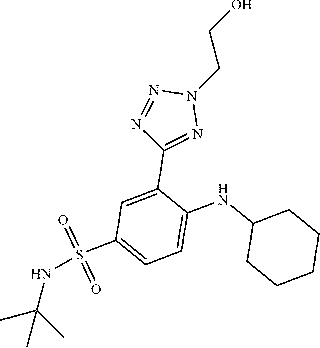 [SMILES: CC(C)(C)NS(=O)(=O)c1ccc(NC2CCCCC2)c(-c2nnn(CCO)n2)c1] |
| 3 | 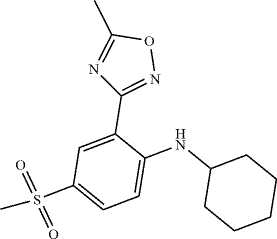 [SMILES: Cc1nc(-c2cc(S(C)(=O)=O)ccc2NC2CCCCC2)no1] |
| 4 | 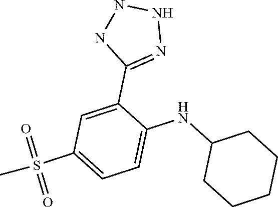 [SMILES: CS(=O)(=O)c1ccc(NC2CCCCC2)c(C2=NNNN2)c1] |
| 5 | 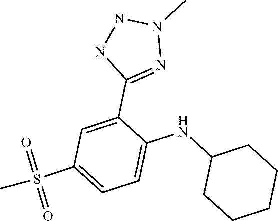 [SMILES: CN1N=C(c2cc(S(C)(=O)=O)ccc2NC2CCCCC2)NN1] |
| 6 | 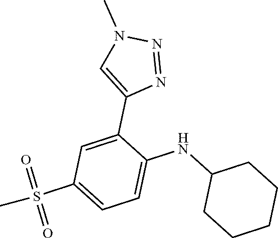 [SMILES: Cn1cc(-c2cc(S(C)(=O)=O)ccc2NC2CCCCC2)nn1] |

| | |
|---|---|
| 7 | 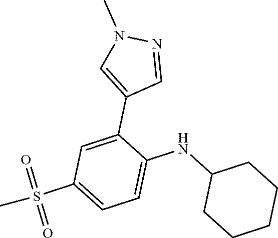 [SMILES: Cn1cc(-c2cc(S(C)(=O)=O)ccc2NC2CCCCC2)cn1] |
| 8 | 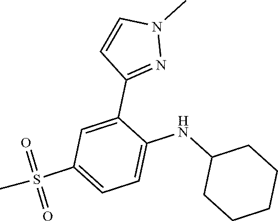 [SMILES: Cn1ccc(-c2cc(S(C)(=O)=O)ccc2NC2CCCCC2)n1] |
| 9 | 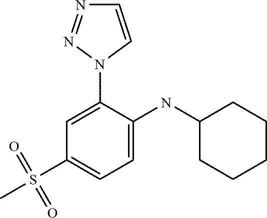 [SMILES: CS(=O)(=O)c1ccc(NC2CCCCC2)c(-n2ccnn2)c1] |
| 10 | 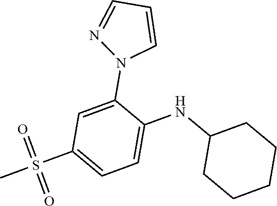 [SMILES: CS(=O)(=O)c1ccc(NC2CCCCC2)c(-n2cccn2)c1] |
| 11 | 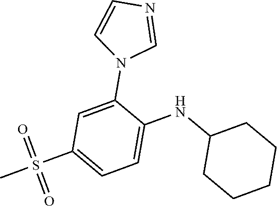 [SMILES: CS(=O)(=O)c1ccc(NC2CCCCC2)c(-n2ccnc2)c1] |
| 12 | 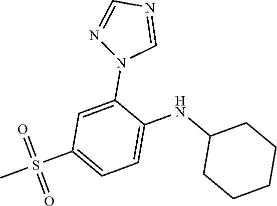 [SMILES: CS(=O)(=O)c1ccc(NC2CCCCC2)c(-n2cncn2)c1] |
| 13 | 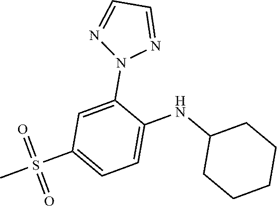 [SMILES: CS(=O)(=O)c1ccc(NC2CCCCC2)c(-n2nccn2)c1] |

| | |
|---|---|
| 14 | 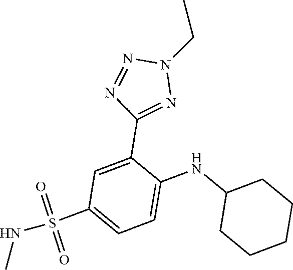 [SMILES: CCn1nnc(-c2cc(S(=O)(=O)NC)ccc2NC2CCCCC2)n1] |
| 15 | 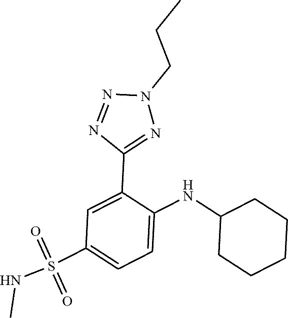 [SMILES: CCCn1nnc(-c2cc(S(=O)(=O)NC)ccc2NC2CCCCC2)n1] |
| 16 | 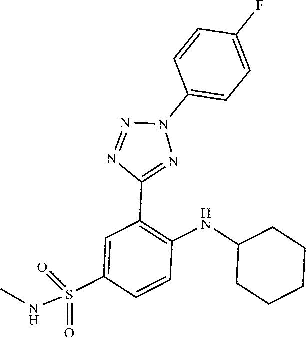 [SMILES: CNS(=O)(=O)c1ccc(NC2CCCCC2)c(-c2nnn(-c3ccc(F)cc3)n2)c1] |
| 17 | 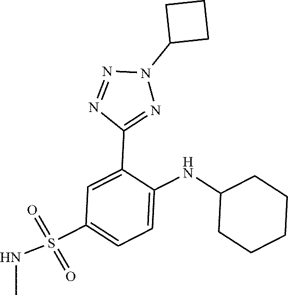 [SMILES: CNS(=O)(=O)c1ccc(NC2CCCCC2)c(-c2nnn(C3CCC3)n2)c1] |
| 18 | 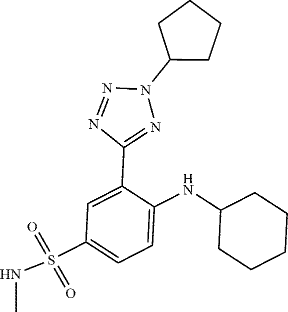 [SMILES: CNS(=O)(=O)c1ccc(NC2CCCCC2)c(-c2nnn(C3CCCC3)n2)c1] |

| | |
|---|---|
| 19 | 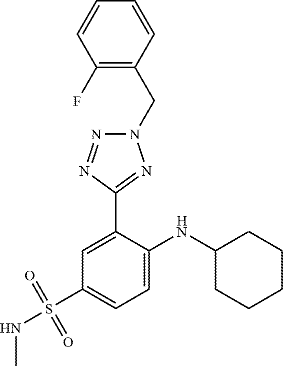 [SMILES: CNS(=O)(=O)c1ccc(NC2CCCCC2)c(-c2nnn(Cc3ccccc3F)n2)c1] |
| 20 | 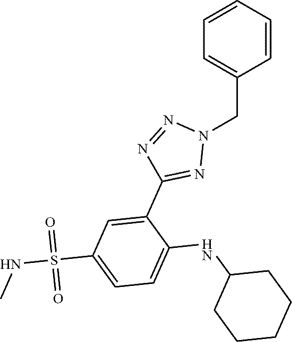 [SMILES: CNS(=O)(=O)c1ccc(NC2CCCCC2)c(-c2nnn(Cc3ccccc3)n2)c1] |
| 21 | 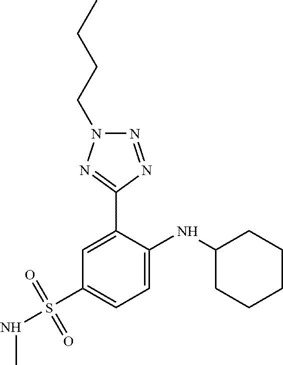 [SMILES: CCCCn1nnc(-c2cc(S(=O)(=O)NC)ccc2NC2CCCCC2)n1] |
| 22 | 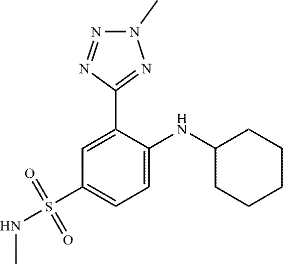 [SMILES: CNS(=O)(=O)c1ccc(NC2CCCCC2)c(-c2nnn(C)n2)c1] |
| 23 | 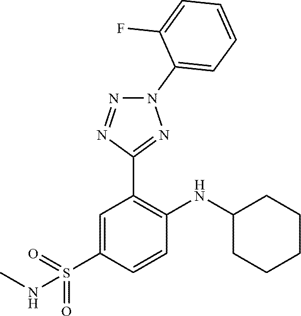 [SMILES: CNS(=O)(=O)c1ccc(NC2CCCCC2)c(-c2nnn(-c3ccccc3F)n2)c1] |

| | |
|---|---|
| 24 | 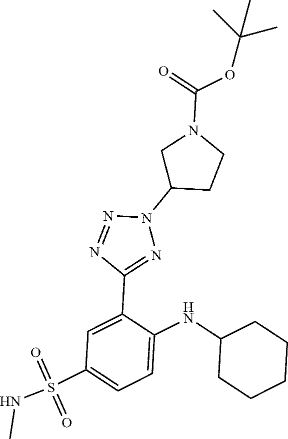 [SMILES: CNS(=O)(=O)c1ccc(NC2CCCCC2)c(-c2nnn(C3CCN(C(=O)OC(C)(C)C)C3)n2)c1] |
| 25 | 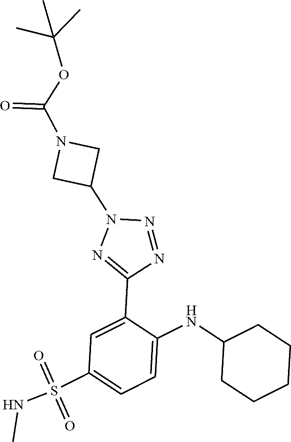 [SMILES: CNS(=O)(=O)c1ccc(NC2CCCCC2)c(-c2nnn(C3CN(C(=O)OC(C)(C)C)C3)n2)c1] |
| 26 | 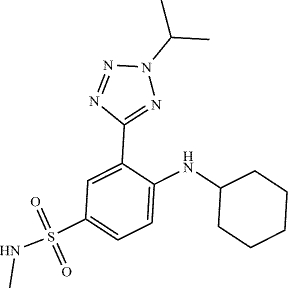 [SMILES: CNS(=O)(=O)c1ccc(NC2CCCCC2)c(-c2nnn(C(C)C)n2)c1] |
| 27 | 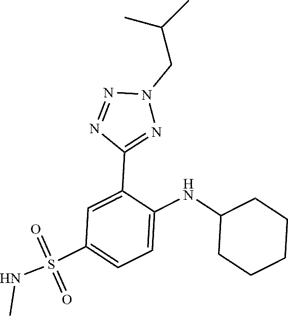 [SMILES: CNS(=O)(=O)c1ccc(NC2CCCCC2)c(-c2nnn(CC(C)C)n2)c1] |

| | |
|---|---|
| 28 | 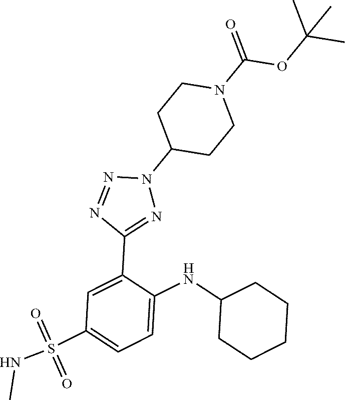 [SMILES: CNS(=O)(=O)c1ccc(NC2CCCCC2)c(-c2nnn(C3CCN(C(=O)OC(C)(C)C)CC3)n2)c1] |
| 29 | 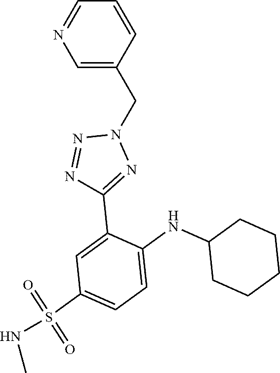 [SMILES: CNS(=O)(=O)c1ccc(NC2CCCCC2)c(-c2nnn(Cc3cccnc3)n2)c1] |
| 30 | 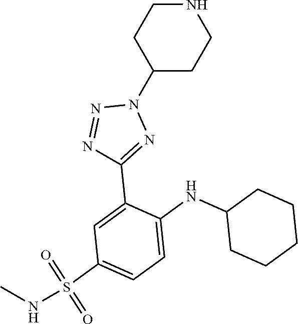 [SMILES: CNS(=O)(=O)c1ccc(NC2CCCCC2)c(-c2nnn(C3CCNCC3)n2)c1] |
| 31 | 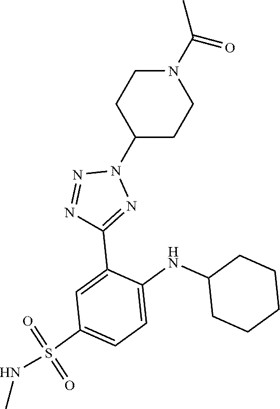 [SMILES: CNS(=O)(=O)c1ccc(NC2CCCCC2)c(-c2nnn(C3CCN(C(C)=O)CC3)n2)c1] |

| | |
|---|---|
| 32 | 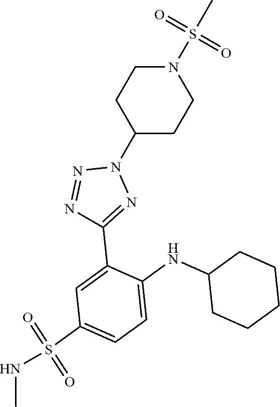 [SMILES: CNS(=O)(=O)c1ccc(NC2CCCCC2)c(-c2nnn(C3CCN(S(C)(=O)=O)CC3)n2)c1] |
| 33 | 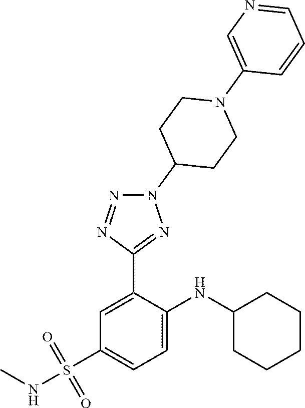 [SMILES: CNS(=O)(=O)c1ccc(NC2CCCCC2)c(-c2nnn(C3CCN(c4cccnc4)CC3)n2)c1] |
| 34 | 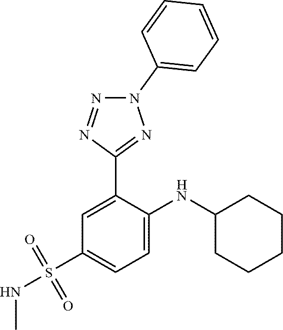 [SMILES: CNS(=O)(=O)c1ccc(NC2CCCCC2)c(-c2nnn(-c3ccccc3)n2)c1] |
| 35 | 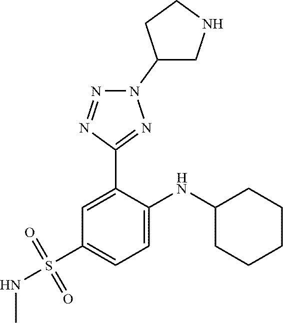 [SMILES: CNS(=O)(=O)c1ccc(NC2CCCCC2)c(-c2nnn(C3CCNC3)n2)c1] |

| | |
|---|---|
| 36 | 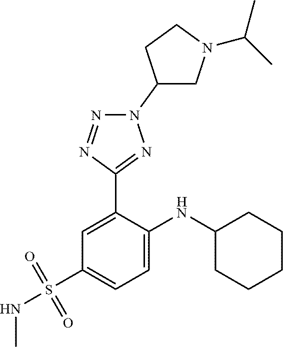 [SMILES: CNS(=O)(=O)c1ccc(NC2CCCCC2)c(-c2nnn(C3CCN(C(C)C)C3)n2)c1] |
| 37 | 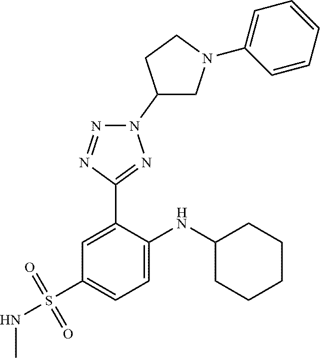 [SMILES: CNS(=O)(=O)c1ccc(NC2CCCCC2)c(-c2nnn(C3CCN(c4ccccc4)C3)n2)c1] |
| 38 | 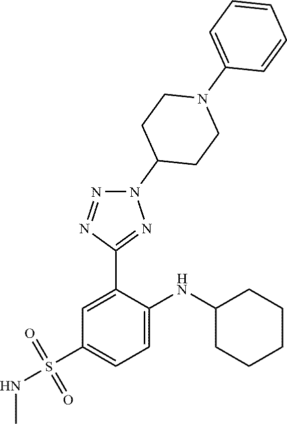 [SMILES: CNS(=O)(=O)c1ccc(NC2CCCCC2)c(-c2nnn(C3CCN(c4ccccc4)CC3)n2)c1] |
| 39 | 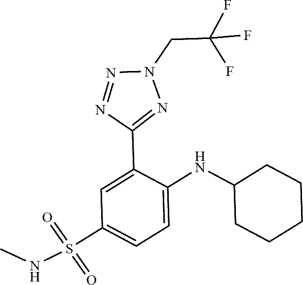 [SMILES: CNS(=O)(=O)c1ccc(NC2CCCCC2)c(-c2nnn(CC(F)(F)F)n2)c1] |

| | |
|---|---|
| 40 | 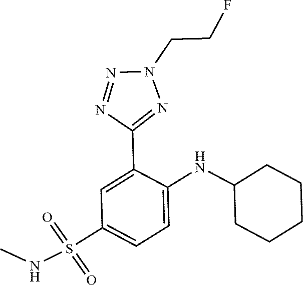 [SMILES: CNS(=O)(=O)c1ccc(NC2CCCCC2)c(-c2nnn(CCF)n2)c1] |
| 41 | 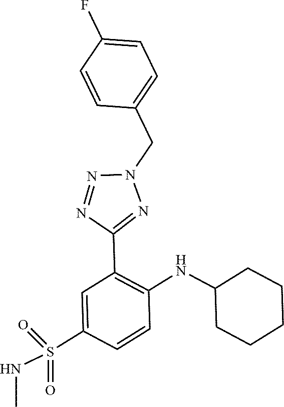 [SMILES: CNS(=O)(=O)c1ccc(NC2CCCCC2)c(-c2nnn(Cc3ccc(F)cc3)n2)c1] |
| 42 | 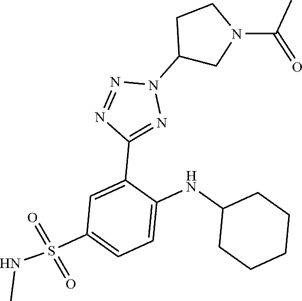 [SMILES: CNS(=O)(=O)c1ccc(NC2CCCCC2)c(-c2nnn(C3CCN(C(C)=O)C3)n2)c1] |
| 43 | 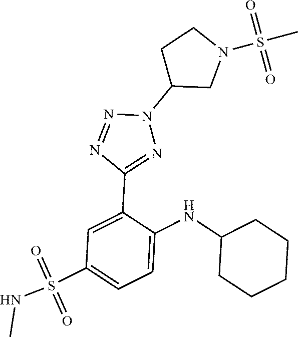 |
| 44 | 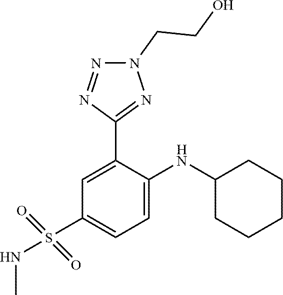 [SMILES: CNS(=O)(=O)c1ccc(NC2CCCCC2)c(-c2nnn(CCO)n2)c1] |

| | |
|---|---|
| 45 | 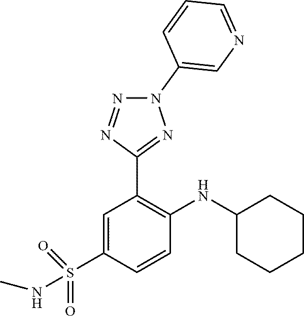 [SMILES: CNS(=O)(=O)c1ccc(NC2CCCCC2)c(-c2nnn(-c3cccnc3)n2)c1] |
| 46 | 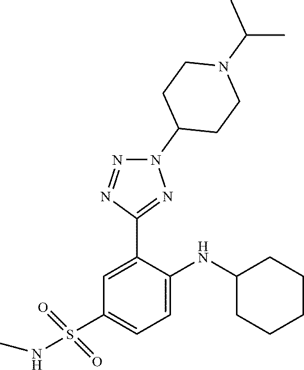 [SMILES: CNS(=O)(=O)c1ccc(NC2CCCCC2)c(-c2nnn(C3CCN(C(C)C)CC3)n2)c1] |
| 47 | 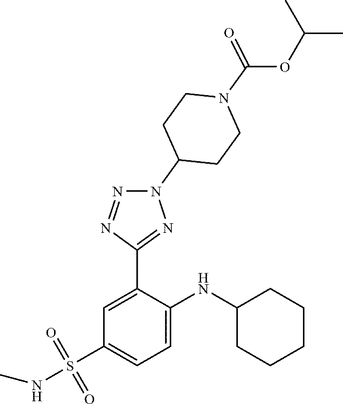 [SMILES: CNS(=O)(=O)c1ccc(NC2CCCCC2)c(-c2nnn(C3CCN(C(=O)OC(C)C)CC3)n2)c1] |
| 48 | 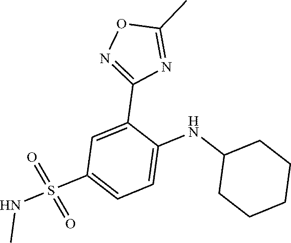 [SMILES: CNS(=O)(=O)c1ccc(NC2CCCCC2)c(-c2noc(C)n2)c1] |

| | |
|---|---|
| 49 | 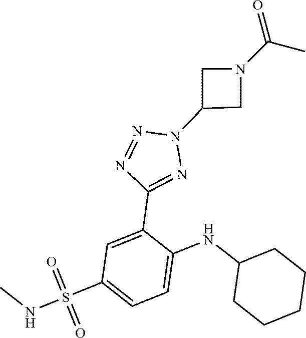 [SMILES: CNS(=O)(=O)c1ccc(NC2CCCCC2)c(-c2nnn(C3CN(C(C)=O)C3)n2)c1] |
| 50 | 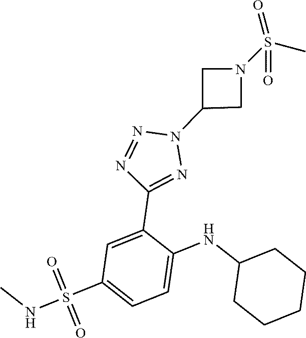 |
| 51 | 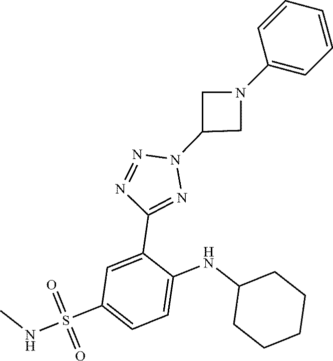 [SMILES: CNS(=O)(=O)c1ccc(NC2CCCCC2)c(-c2nnn(C3CN(c4ccccc4)C3)n2)c1] |
| 52 | 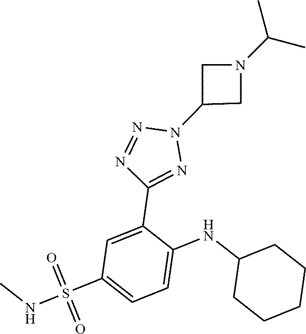 [SMILES: CNS(=O)(=O)c1ccc(NC2CCCCC2)c(-c2nnn(C3CN(C(C)C)C3)n2)c1] |

| | |
|---|---|
| 53 | 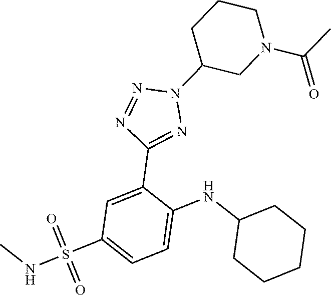 [SMILES: CNS(=O)(=O)c1ccc(NC2CCCCC2)c(-c2nnn(C3CCCN(C(C)=O)C3)n2)c1] |
| 54 | 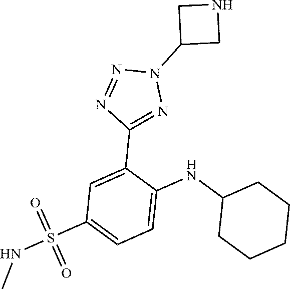 [SMILES: CNS(=O)(=O)c1ccc(NC2CCCCC2)c(-c2nnn(C3CNC3)n2)c1] |
| 55 | 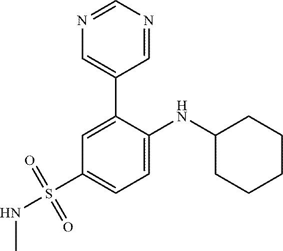 [SMILES: CNS(=O)(=O)c1ccc(NC2CCCCC2)c(-c2cncnc2)c1] |
| 56 | 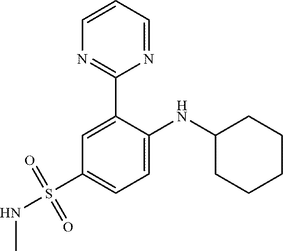 [SMILES: CNS(=O)(=O)c1ccc(NC2CCCCC2)c(-c2ncccn2)c1] |
| 57 | 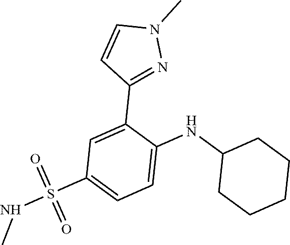 [SMILES: CNS(=O)(=O)c1ccc(NC2CCCCC2)c(-c2ccn(C)n2)c1] |
| 58 | 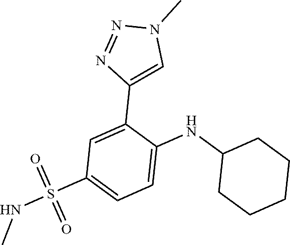 [SMILES: CNS(=O)(=O)c1ccc(NC2CCCCC2)c(-c2cn(C)nn2)c1] |

| | |
|---|---|
| 59 | 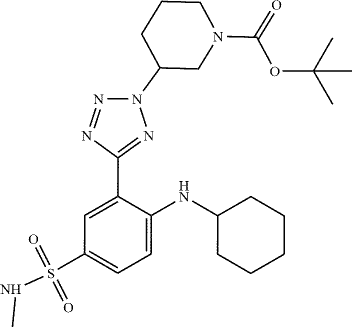 [SMILES: CNS(=O)(=O)c1ccc(NC2CCCCC2)c(-c2nnn(C3CCCN(C(=O)OC(C)(C)C)C3)n2)c1] |
| 60 | 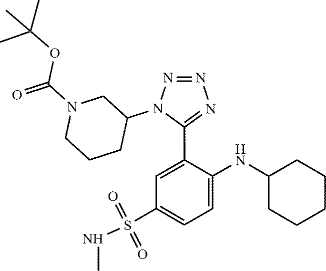 [SMILES: CNS(=O)(=O)c1ccc(NC2CCCCC2)c(-c2nnnn2C2CCCN(C(=O)OC(C)(C)C)C2)c1] |
| 61 | 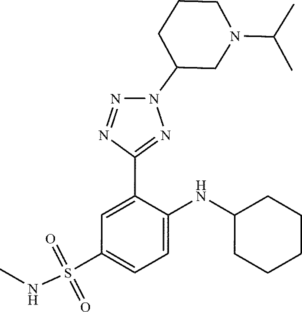 [SMILES: CNS(=O)(=O)c1ccc(NC2CCCCC2)c(-c2nnn(C3CCCN(C(C)C)C3)n2)c1] |
| 62 | 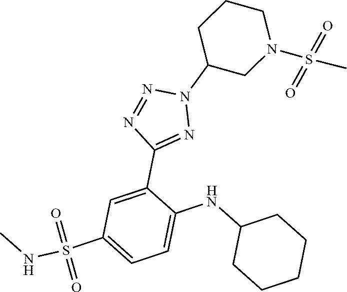 |
| 63 | 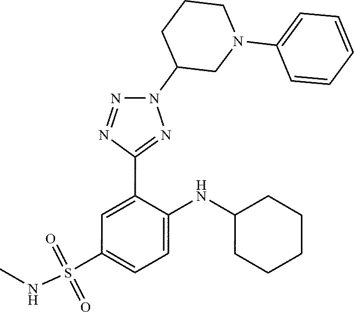 [SMILES: CNS(=O)(=O)c1ccc(NC2CCCCC2)c(-c2nnn(C3CCCN(c4ccccc4)C3)n2)c1] |

| | |
|---|---|
| 64 | 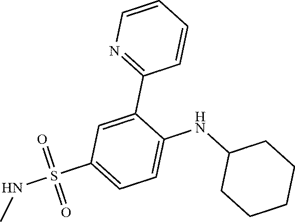 [SMILES: CNS(=O)(=O)c1ccc(NC2CCCCC2)c(-c2ccccn2)c1] |
| 65 | 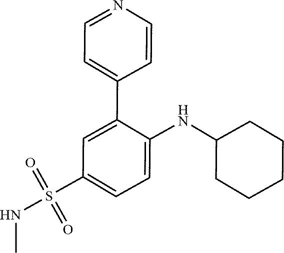 [SMILES: CNS(=O)(=O)c1ccc(NC2CCCCC2)c(-c2ccncc2)c1] |
| 66 | 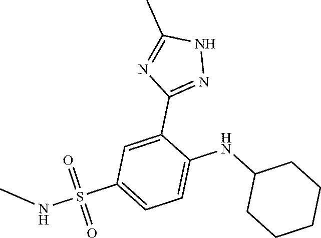 [SMILES: CNS(=O)(=O)c1ccc(NC2CCCCC2)c(-c2n[nH]c(C)n2)c1] |
| 67 | 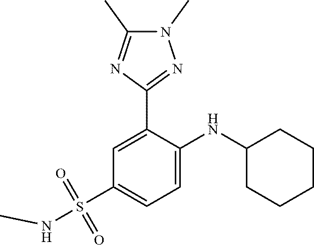 [SMILES: CNS(=O)(=O)c1ccc(NC2CCCCC2)c(-c2nc(C)n(C)n2)c1] |
| 68 | 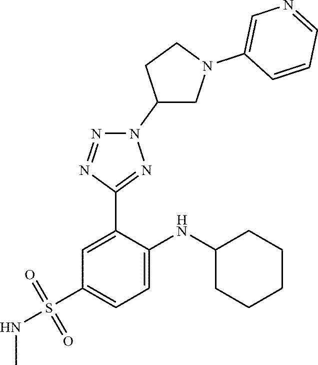 [SMILES: CNS(=O)(=O)c1ccc(NC2CCCCC2)c(-c2nnn(C3CCN(c4cccnc4)C3)n2)c1] |
| 69 | 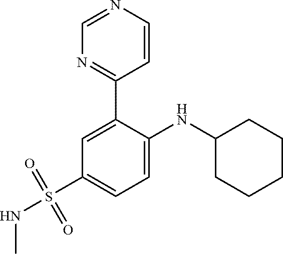 [SMILES: CNS(=O)(=O)c1ccc(NC2CCCCC2)c(-c2ccncn2)c1] |

| | |
|---|---|
| 70 | 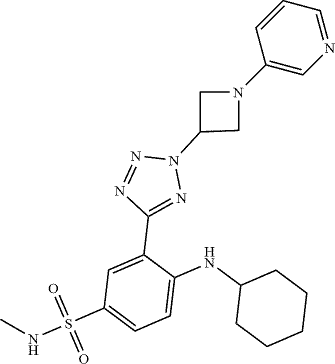 [SMILES: CNS(=O)(=O)c1ccc(NC2CCCCC2)c(-c2nnn(C3CN(c4cccnc4)C3)n2)c1] |
| 71 | 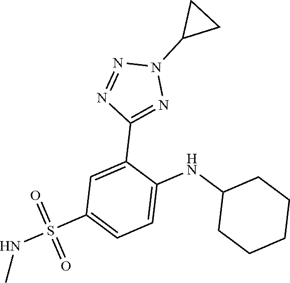 [SMILES: CNS(=O)(=O)c1ccc(NC2CCCCC2)c(-c2nnn(C3CC3)n2)c1] |
| 72 | 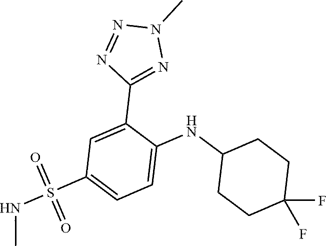 [SMILES: CNS(=O)(=O)c1ccc(NC2CCC(F)(F)CC2)c(-c2nnn(C)n2)c1] |
| 73 | 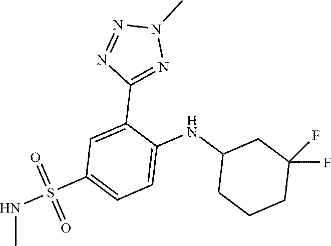 [SMILES: CNS(=O)(=O)c1ccc(NC2CCCC(F)(F)C2)c(-c2nnn(C)n2)c1] |
| 74 | 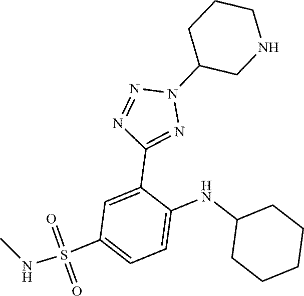 [SMILES: CNS(=O)(=O)c1ccc(NC2CCCCC2)c(-c2nnn(C3CCCNC3)n2)c1] |

| | |
|---|---|
| 75 | 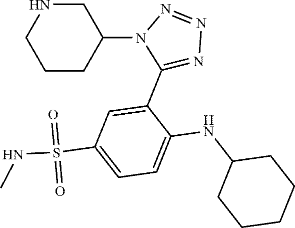 [SMILES: CNS(=O)(=O)c1ccc(NC2CCCCC2)c(-c2nnnn2C2CCCNC2)c1] |
| 76 | 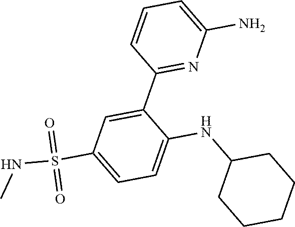 [SMILES: CNS(=O)(=O)c1ccc(NC2CCCCC2)c(-c2cccc(N)n2)c1] |
| 77 | 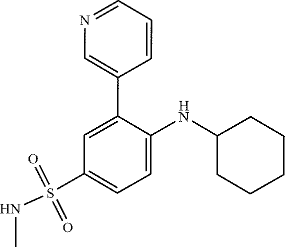 [SMILES: CNS(=O)(=O)c1ccc(NC2CCCCC2)c(-c2cccnc2)c1] |
| 78 | 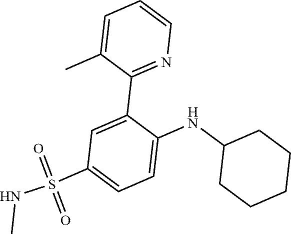 [SMILES: CNS(=O)(=O)c1ccc(NC2CCCCC2)c(-c2ncccc2C)c1] |
| 79 | 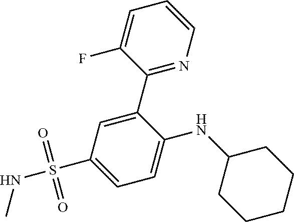 [SMILES: CNS(=O)(=O)c1ccc(NC2CCCCC2)c(-c2ncccc2F)c1] |
| 80 | 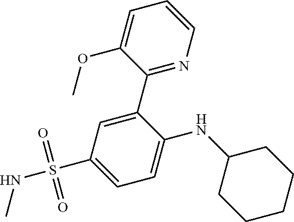 [SMILES: CNS(=O)(=O)c1ccc(NC2CCCCC2)c(-c2ncccc2OC)c1] |
| 81 | 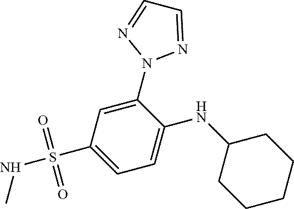 [SMILES: CNS(=O)(=O)c1ccc(NC2CCCCC2)c(-n2nccn2)c1] |

| | |
|---|---|
| 82 | 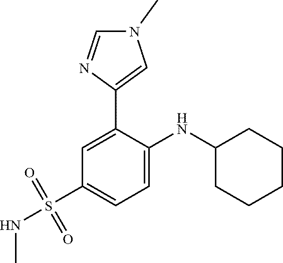 [SMILES: CNS(=O)(=O)c1ccc(NC2CCCCC2)c(-c2cn(C)cn2)c1] |
| 83 | 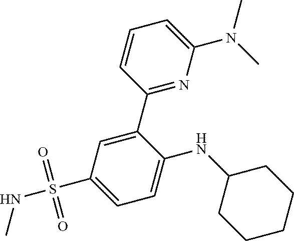 [SMILES: CNS(=O)(=O)c1ccc(NC2CCCCC2)c(-c2cccc(N(C)C)n2)c1] |
| 84 | 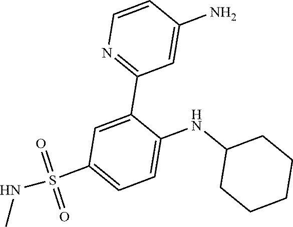 [SMILES: CNS(=O)(=O)c1ccc(NC2CCCCC2)c(-c2cc(N)ccn2)c1] |
| 85 | 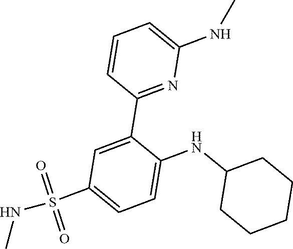 [SMILES: CNc1cccc(-c2cc(S(=O)(=O)NC)ccc2NC2CCCCC2)n1] |
| 86 | 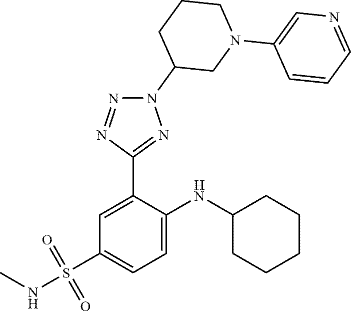 [SMILES: CNS(=O)(=O)c1ccc(NC2CCCCC2)c(-c2nnn(C3CCCN(c4cccnc4)C3)n2)c1] |
| 87 | 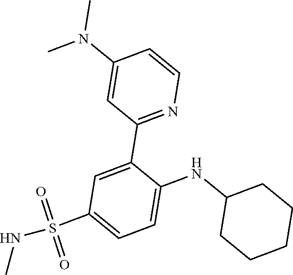 [SMILES: CNS(=O)(=O)c1ccc(NC2CCCCC2)c(-c2cc(N(C)C)ccn2)c1] |

| | |
|---|---|
| 88 | 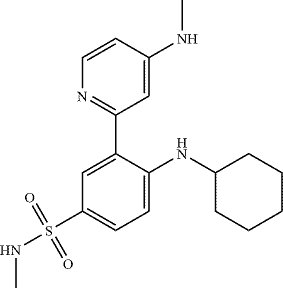 [SMILES: CNc1ccnc(-c2cc(S(=O)(=O)NC)ccc2NC2CCCCC2)c1] |
| 89 | 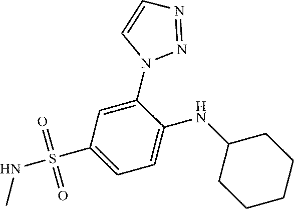 [SMILES: CNS(=O)(=O)c1ccc(NC2CCCCC2)c(-n2ccnn2)c1] |
| 90 | 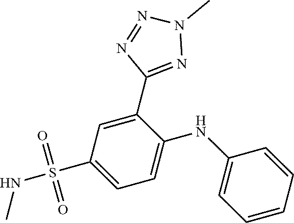 [SMILES: CNS(=O)(=O)c1ccc(Nc2ccccc2)c(-c2nnn(C)n2)c1] |
| 91 | 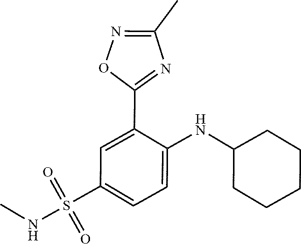 [SMILES: CNS(=O)(=O)c1ccc(NC2CCCCC2)c(-c2nc(C)no2)c1] |
| 92 | 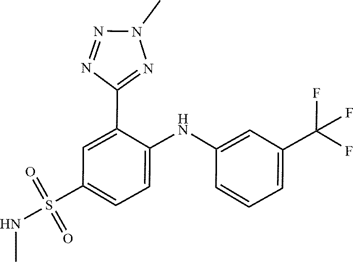 [SMILES: CNS(=O)(=O)c1ccc(Nc2cccc(C(F)(F)F)c2)c(-c2nnn(C)n2)c1] |
| 93 | 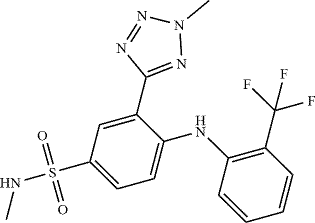 [SMILES: CNS(=O)(=O)c1ccc(Nc2ccccc2C(F)(F)F)c(-c2nnn(C)n2)c1] |

| | |
|---|---|
| 94 | 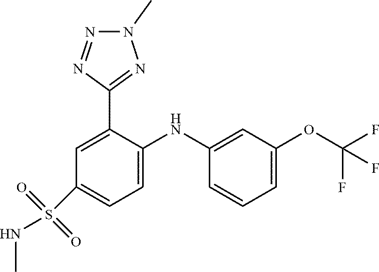 [SMILES: CNS(=O)(=O)c1ccc(Nc2cccc(OC(F)(F)F)c2)c(-c2nnn(C)n2)c1] |
| 95 | 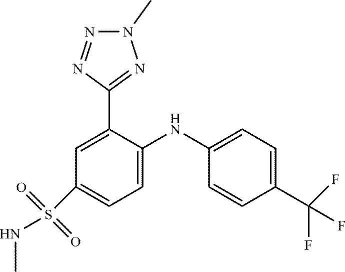 [SMILES: CNS(=O)(=O)c1ccc(Nc2ccc(C(F)(F)F)cc2)c(-c2nnn(C)n2)c1] |
| 96 | 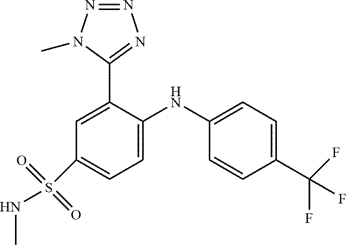 [SMILES: CNS(=O)(=O)c1ccc(Nc2ccc(C(F)(F)F)cc2)c(-c2nnnn2C)c1] |
| 97 | 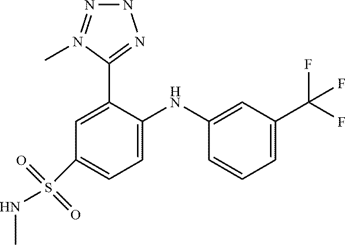 |
| 98 | 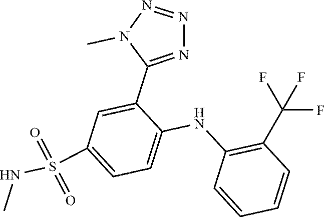 [SMILES: CNS(=O)(=O)c1ccc(Nc2ccccc2C(F)(F)F)c(-c2nnnn2C)c1] |
| 99 | 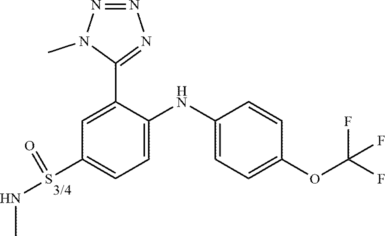 |

| | |
|---|---|
| 100 | 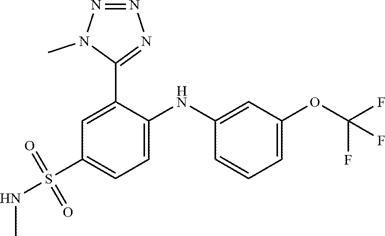 [SMILES: CNS(=O)(=O)c1ccc(Nc2cccc(OC(F)(F)F)c2)c(-c2nnnn2C)c1] |
| 101 | 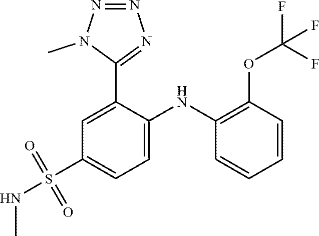 [SMILES: CNS(=O)(=O)c1ccc(Nc2ccccc2OC(F)(F)F)c(-c2nnnn2C)c1] |
| 102 | 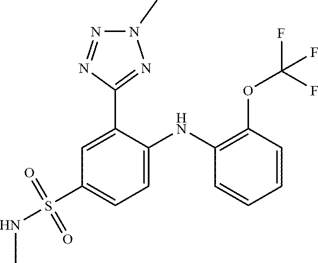 [SMILES: CNS(=O)(=O)c1ccc(Nc2ccccc2OC(F)(F)F)c(-c2nnn(C)n2)c1] |
| 103 | 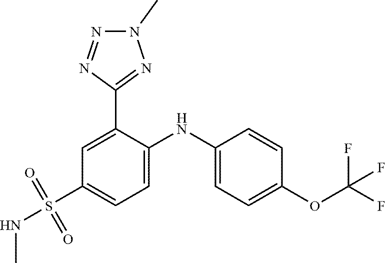 [SMILES: CNS(=O)(=O)c1ccc(Nc2ccc(OC(F)(F)F)cc2)c(-c2nnn(C)n2)c1] |
| 104 | 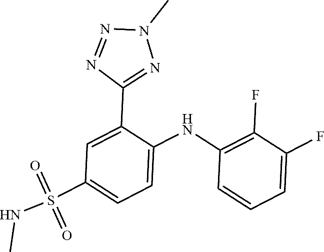 [SMILES: CNS(=O)(=O)c1ccc(Nc2cccc(F)c2F)c(-c2nnn(C)n2)c1] |
| 105 | 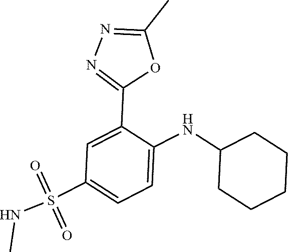 [SMILES: CNS(=O)(=O)c1ccc(NC2CCCCC2)c(-c2nnc(C)o2)c1] |

| | |
|---|---|
| 106 | 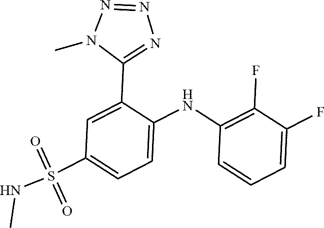 [SMILES: CNS(=O)(=O)c1ccc(Nc2cccc(F)c2F)c(-c2nnnn2C)c1] |
| 107 | 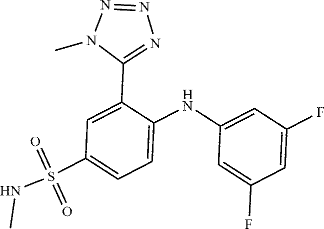 [SMILES: CNS(=O)(=O)c1ccc(Nc2cc(F)cc(F)c2)c(-c2nnnn2C)c1] |
| 108 | 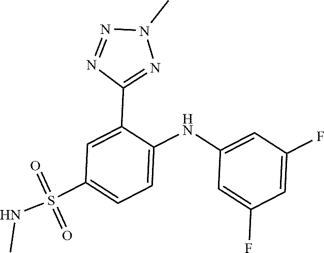 [SMILES: CNS(=O)(=O)c1ccc(Nc2cc(F)cc(F)c2)c(-c2nnn(C)n2)c1] |
| 109 | 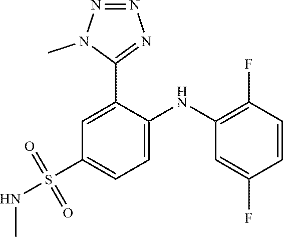 [SMILES: CNS(=O)(=O)c1ccc(Nc2cc(F)ccc2F)c(-c2nnnn2C)c1] |
| 110 | 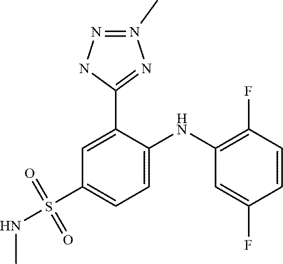 |
| 111 | 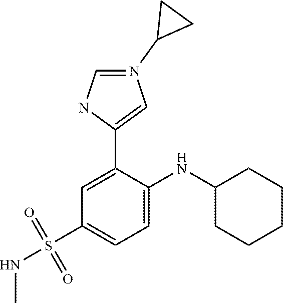 |

| | |
|---|---|
| 112 | 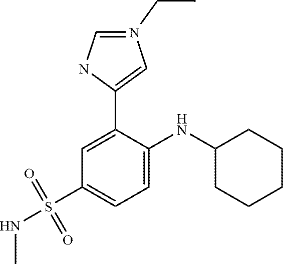 |
| 113 | 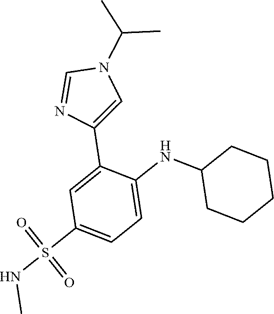 [SMILES: CNS(=O)(=O)c1ccc(NC2CCCCC2)c(-c2cn(C(C)C)cn2)c1] |
| 114 | 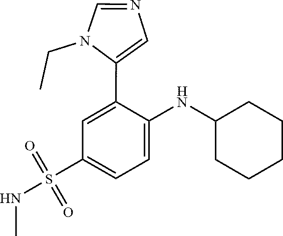 [SMILES: CCn1cncc1-c1cc(S(=O)(=O)NC)ccc1NC1CCCCC1] |
| 115 | 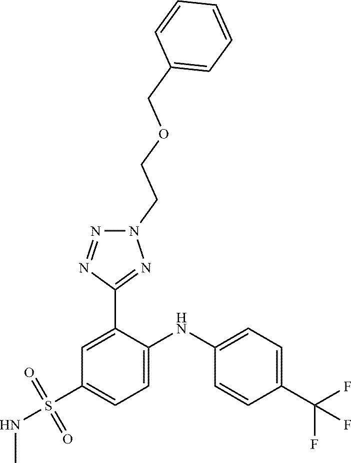 [SMILES: CNS(=O)(=O)c1ccc(Nc2ccc(C(F)(F)F)cc2)c(-c2nnn(CCOCc3ccccc3)n2)c1] |
| 116 | 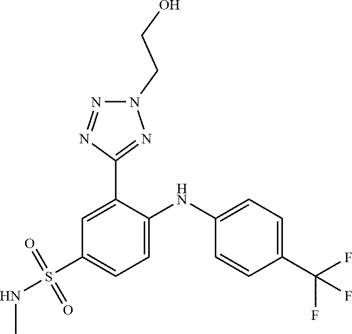 [SMILES: CNS(=O)(=O)c1ccc(Nc2ccc(C(F)(F)F)cc2)c(-c2nnn(CCO)n2)c1] |

| | |
|---|---|
| 117 | 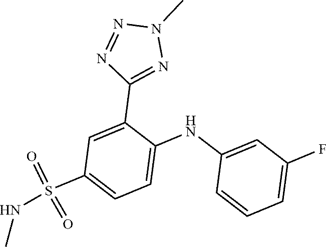 [SMILES: CNS(=O)(=O)c1ccc(Nc2cccc(F)c2)c(-c2nnn(C)n2)c1] |
| 118 | 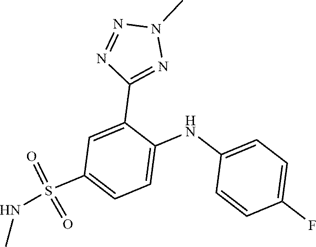 [SMILES: CNS(=O)(=O)c1ccc(Nc2ccc(F)cc2)c(-c2nnn(C)n2)c1] |
| 119 | 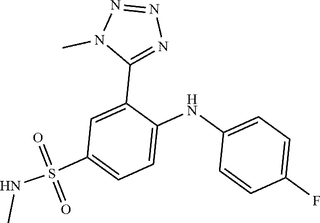 [SMILES: CNS(=O)(=O)c1ccc(Nc2ccc(F)cc2)c(-c2nnnn2C)c1] |
| 120 | 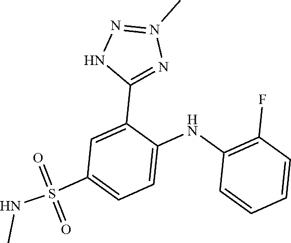 |
| 121 | 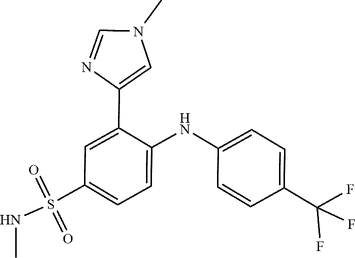 [SMILES: CNS(=O)(=O)c1ccc(Nc2ccc(C(F)(F)F)cc2)c(-c2cn(C)cn2)c1] |
| 122 | 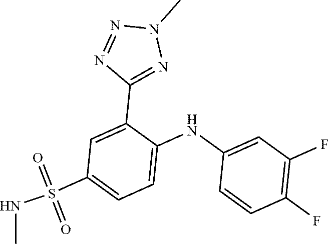 [SMILES: CNS(=O)(=O)c1ccc(Nc2ccc(F)c(F)c2)c(-c2nnn(C)n2)c1] |

| | |
|---|---|
| 123 | 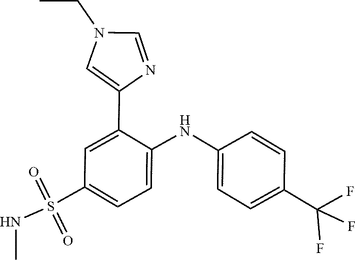 [SMILES: CCn1cnc(-c2cc(S(=O)(=O)NC)ccc2Nc2ccc(C(F)(F)F)cc2)c1] |
| 124 | 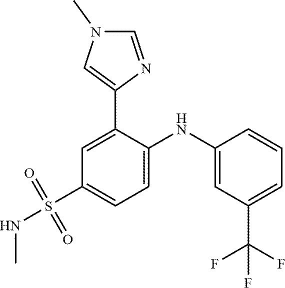 [SMILES: CNS(=O)(=O)c1ccc(Nc2cccc(C(F)(F)F)c2)c(-c2cn(C)cn2)c1] |
| 125 | 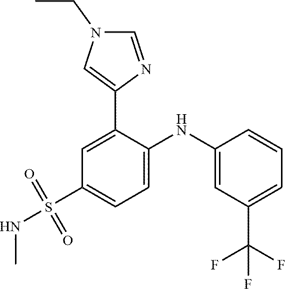 [SMILES: CCn1cnc(-c2cc(S(=O)(=O)NC)ccc2Nc2cccc(C(F)(F)F)c2)c1] |
| 126 | 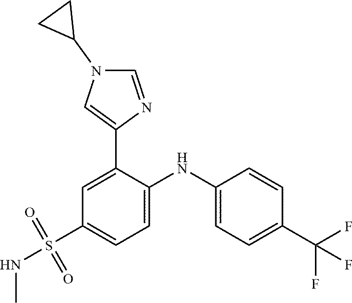 [SMILES: CNS(=O)(=O)c1ccc(Nc2ccc(C(F)(F)F)cc2)c(-c2cn(C3CC3)cn2)c1] |
| 127 | 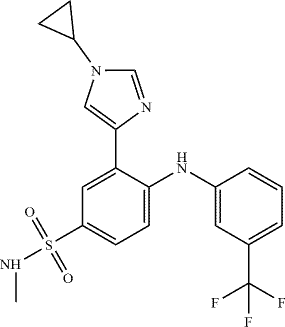 [SMILES: CNS(=O)(=O)c1ccc(Nc2cccc(C(F)(F)F)c2)c(-c2cn(C3CC3)cn2)c1] |

| | |
|---|---|
| 128 | 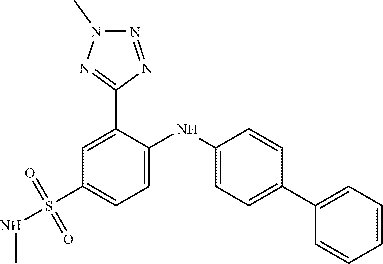 [SMILES: CNS(=O)(=O)c1ccc(Nc2ccc(-c3ccccc3)cc2)c(-c2nnn(C)n2)c1] |
| 129 | 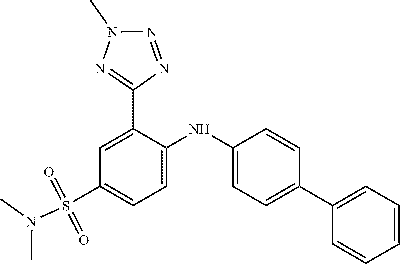 [SMILES: CN(C)S(=O)(=O)c1ccc(Nc2ccc(-c3ccccc3)cc2)c(-c2nnn(C)n2)c1] |
| 130 | 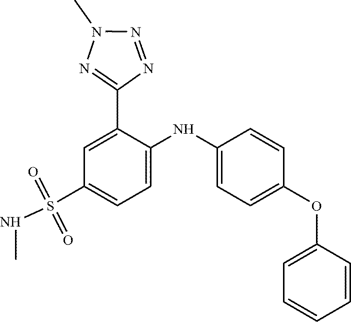 [SMILES: CNS(=O)(=O)c1ccc(Nc2ccc(Oc3ccccc3)cc2)c(-c2nnn(C)n2)c1] |
| 131 | 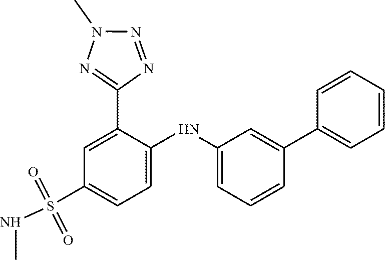 [SMILES: CNS(=O)(=O)c1ccc(Nc2cccc(-c3ccccc3)c2)c(-c2nnn(C)n2)c1] |
| 132 | 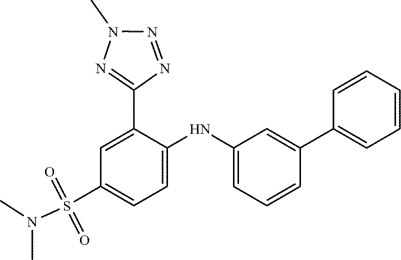 [SMILES: CN(C)S(=O)(=O)c1ccc(Nc2cccc(-c3ccccc3)c2)c(-c2nnn(C)n2)c1] |
| 133 | 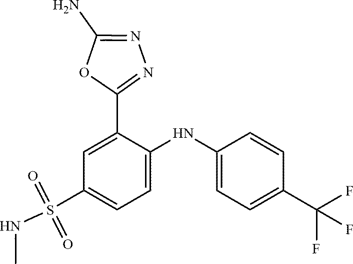 [SMILES: CNS(=O)(=O)c1ccc(Nc2ccc(C(F)(F)F)cc2)c(-c2nnc(N)o2)c1] |

| | |
|---|---|
| 134 | 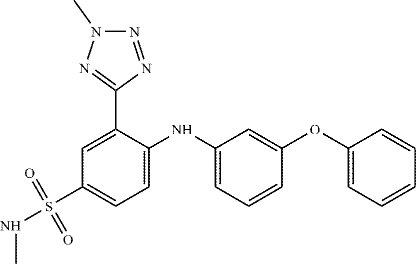 [SMILES: CNS(=O)(=O)c1ccc(Nc2cccc(Oc3ccccc3)c2)c(-c2nnn(C)n2)c1] |
| 135 | 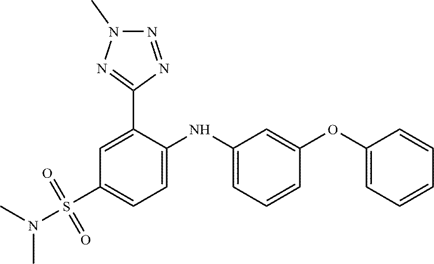 [SMILES: CN(C)S(=O)(=O)c1ccc(Nc2cccc(Oc3ccccc3)c2)c(-c2nnn(C)n2)c1] |
| 136 | 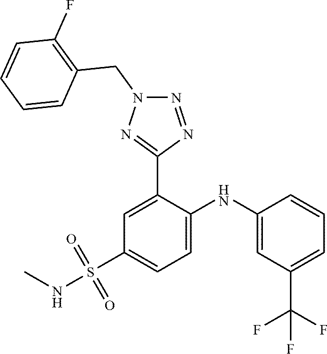 [SMILES: CNS(=O)(=O)c1ccc(Nc2cccc(C(F)(F)F)c2)c(-c2nnn(Cc3ccccc3F)n2)c1] |
| 137 | 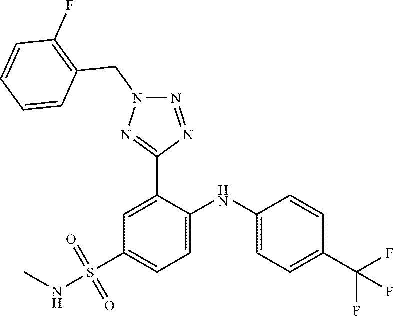 [SMILES: CNS(=O)(=O)c1ccc(Nc2ccc(C(F)(F)F)cc2)c(-c2nnn(Cc3ccccc3F)n2)c1] |
| 138 | 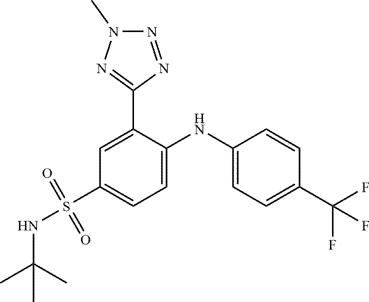 [SMILES: Cn1nnc(-c2cc(S(=O)(=O)NC(C)(C)C)ccc2Nc2ccc(C(F)(F)F)cc2)n1] |

| | |
|---|---|
| 139 | 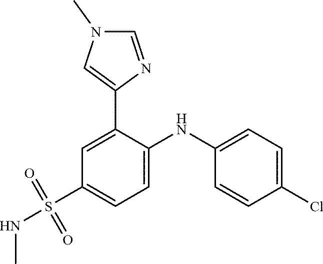 [SMILES: CNS(=O)(=O)c1ccc(Nc2ccc(Cl)cc2)c(-c2cn(C)cn2)c1] |
| 140 | 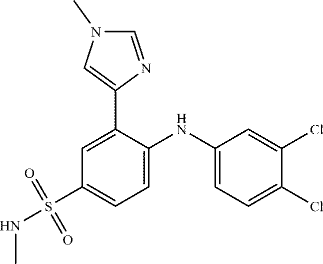 [SMILES: CNS(=O)(=O)c1ccc(Nc2ccc(Cl)c(Cl)c2)c(-c2cn(C)cn2)c1] |
| 141 | 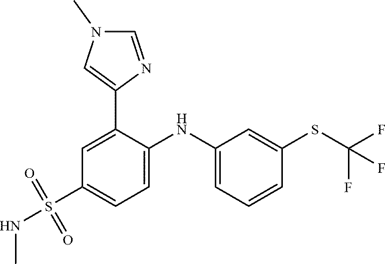 [SMILES: CNS(=O)(=O)c1ccc(Nc2cccc(SC(F)(F)F)c2)c(-c2cn(C)cn2)c1] |
| 142 | 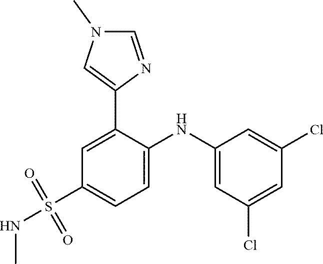 [SMILES: CNS(=O)(=O)c1ccc(Nc2cc(Cl)cc(Cl)c2)c(-c2cn(C)cn2)c1] |
| 143 | 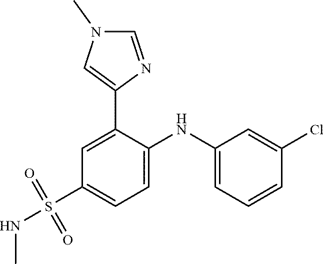 [SMILES: CNS(=O)(=O)c1ccc(Nc2cccc(Cl)c2)c(-c2cn(C)cn2)c1] |
| 144 | 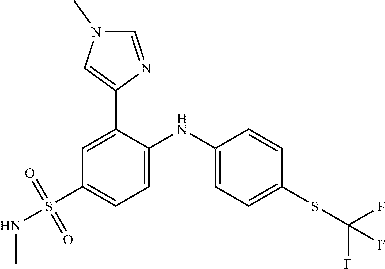 [SMILES: CNS(=O)(=O)c1ccc(Nc2ccc(SC(F)(F)F)cc2)c(-c2cn(C)cn2)c1] |

| | |
|---|---|
| 145 | 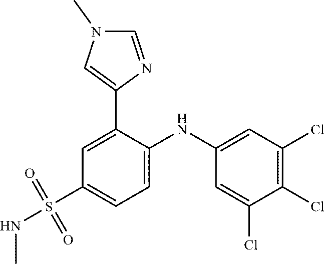 [SMILES: CNS(=O)(=O)c1ccc(Nc2cc(Cl)c(Cl)c(Cl)c2)c(-c2cn(C)cn2)c1] |
| 146 | 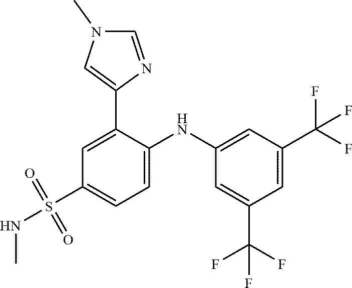 [SMILES: CNS(=O)(=O)c1ccc(Nc2cc(C(F)(F)F)cc(C(F)(F)F)c2)c(-c2cn(C)cn2)c1] |
| 147 | 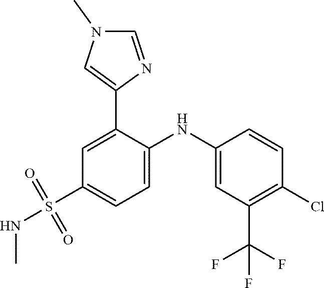 [SMILES: CNS(=O)(=O)c1ccc(Nc2ccc(Cl)c(C(F)(F)F)c2)c(-c2cn(C)cn2)c1] |
| 148 | 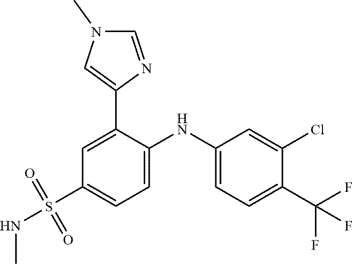 [SMILES: CNS(=O)(=O)c1ccc(Nc2ccc(C(F)(F)F)c(Cl)c2)c(-c2cn(C)cn2)c1] |
| 149 | 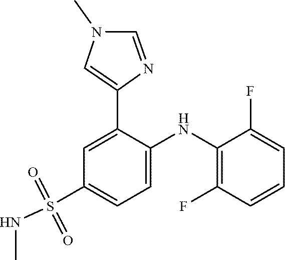 [SMILES: CNS(=O)(=O)c1ccc(Nc2c(F)cccc2F)c(-c2cn(C)cn2)c1] |

| | |
|---|---|
| 150 | 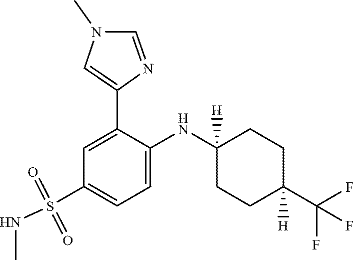 [SMILES: CNS(=O)(=O)c1ccc(N[C@H]2CC[C@@H](C(F)(F)F)CC2)c(-c2cn(C)cn2)c1] |
| 151 | 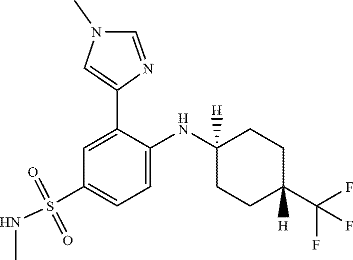 [SMILES: CNS(=O)(=O)c1ccc(N[C@H]2CC[C@H](C(F)(F)F)CC2)c(-c2cn(C)cn2)c1] |
| 152 | 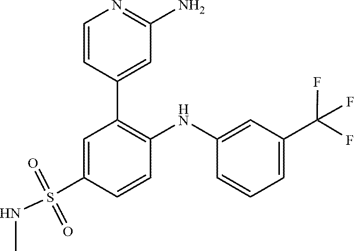 [SMILES: CNS(=O)(=O)c1ccc(Nc2cccc(C(F)(F)F)c2)c(-c2ccnc(N)c2)c1] |
| 153 | 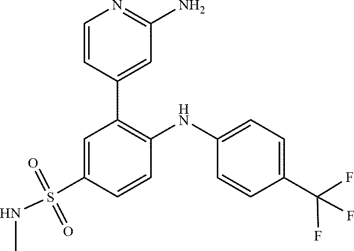 [SMILES: CNS(=O)(=O)c1ccc(Nc2ccc(C(F)(F)F)cc2)c(-c2ccnc(N)c2)c1] |

published Feb. 28, 2019.

### Subgenera of Compounds of Formula (I-B)

In certain embodiments, the compound of Formula (I-B) is of formula:

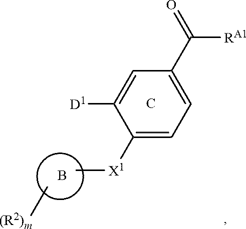

or a pharmaceutically acceptable salt, solvate, hydrate, polymorph, co-crystal, tautomer, stereoisomer, isotopically labeled derivative, or prodrug thereof.

In certain embodiments, the compound of Formula (I-B) is of formula:

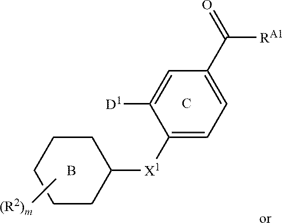

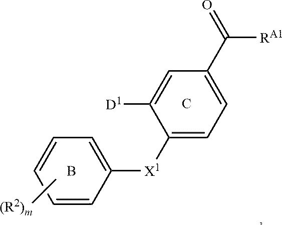

or a pharmaceutically acceptable salt, solvate, hydrate, polymorph, co-crystal, tautomer, stereoisomer, isotopically labeled derivative, or prodrug thereof.

In certain embodiments, the compound of Formula (I-B) is of formula:

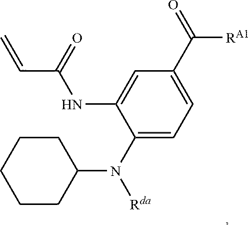

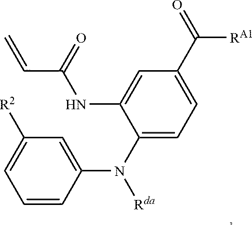

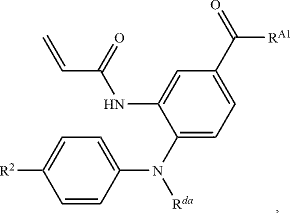

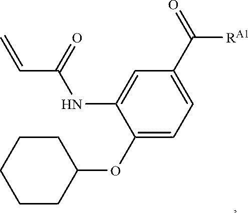

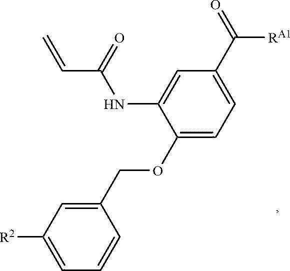

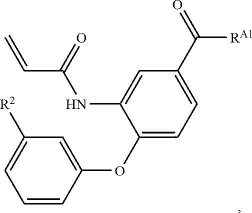

or a pharmaceutically acceptable salt, solvate, hydrate, polymorph, co-crystal, tautomer, stereoisomer, isotopically labeled derivative, or prodrug thereof.

In certain embodiments, the compound of Formula (I-B) is of formula:

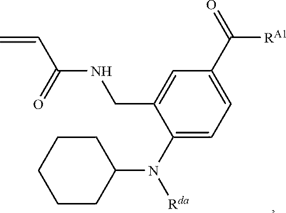

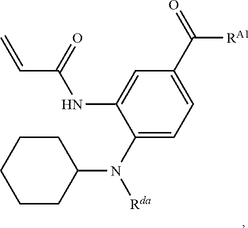

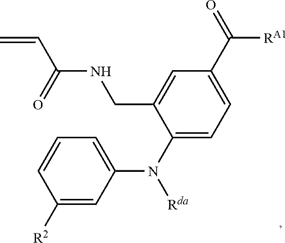

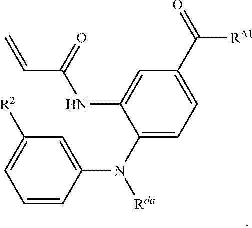

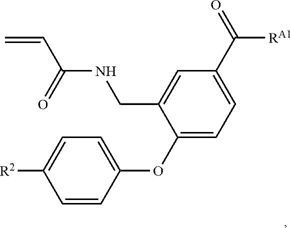

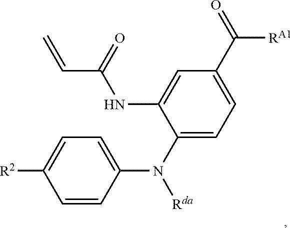

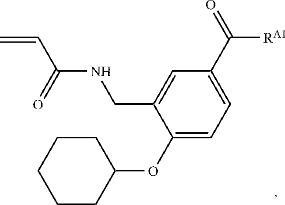

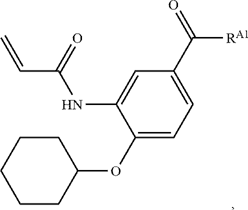

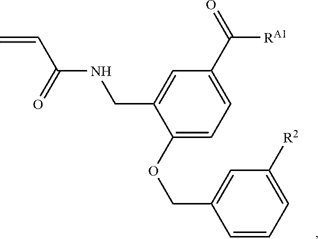

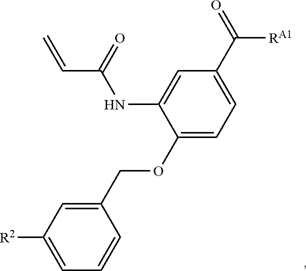

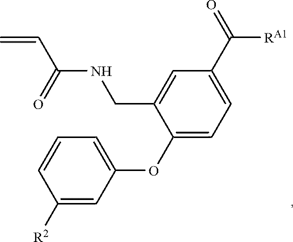

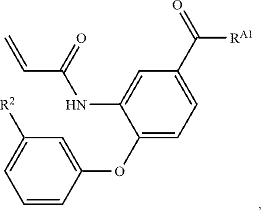

or a pharmaceutically acceptable salt, solvate, hydrate, polymorph, co-crystal, tautomer, stereoisomer, isotopically labeled derivative, or prodrug thereof.

In certain embodiments, the compound of Formula (I-B) is of formula:

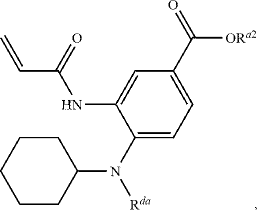

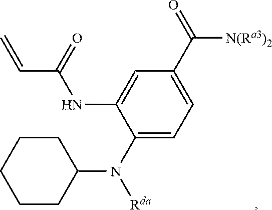

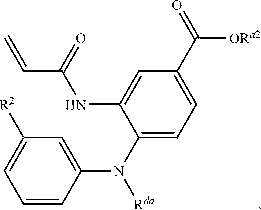

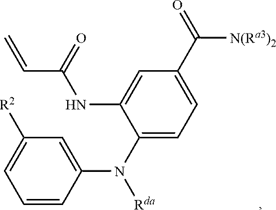

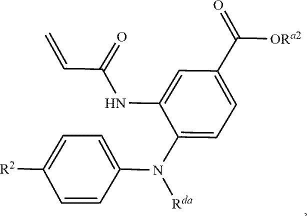

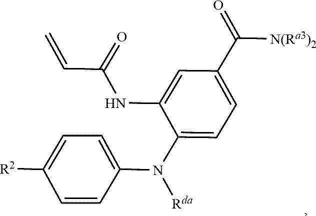

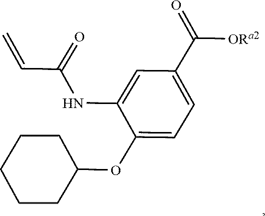

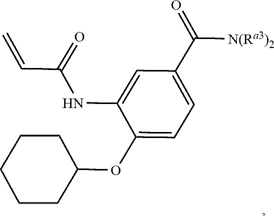

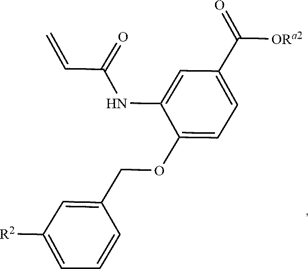

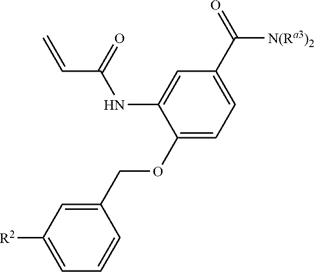

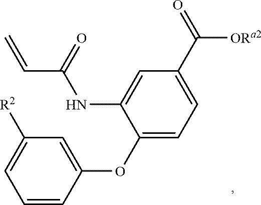

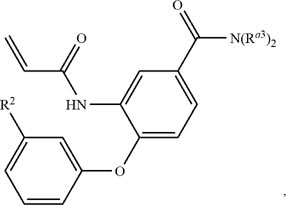

or a pharmaceutically acceptable salt, solvate, hydrate, polymorph, co-crystal, tautomer, stereoisomer, isotopically labeled derivative, or prodrug thereof.

In certain embodiments, the compound of Formula (I-B) is of formula:

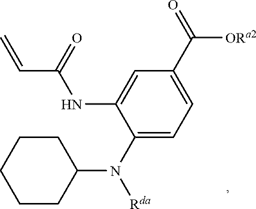

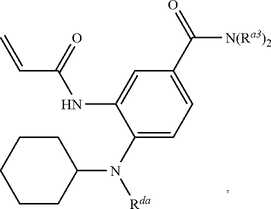

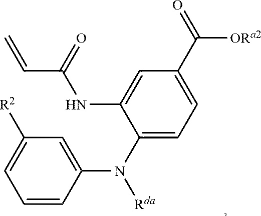

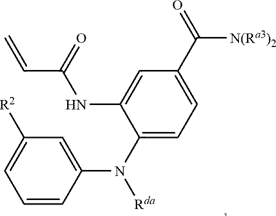

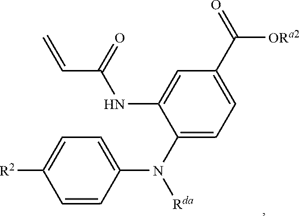

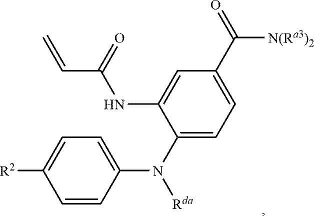

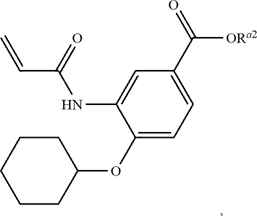

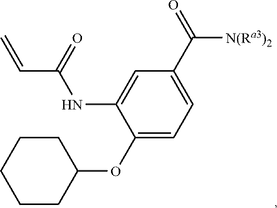

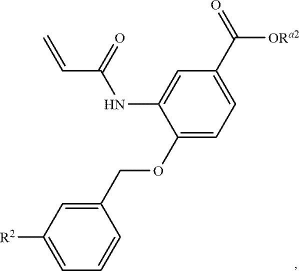

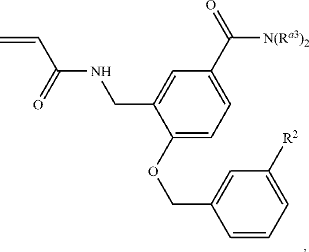

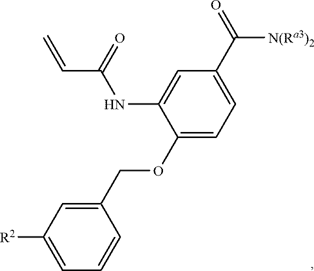

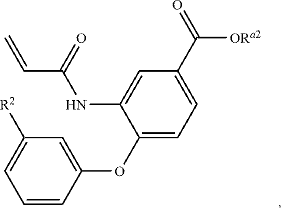

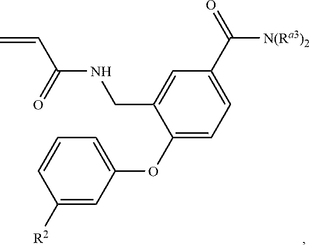

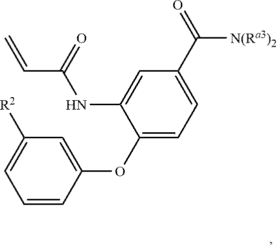

or a pharmaceutically acceptable salt, solvate, hydrate, polymorph, co-crystal, tautomer, stereoisomer, isotopically labeled derivative, or prodrug thereof.

In certain embodiments, the compound of Formula (I-B) is of formula:

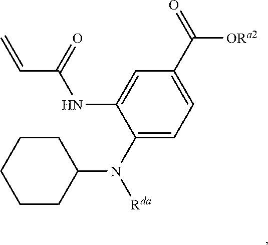

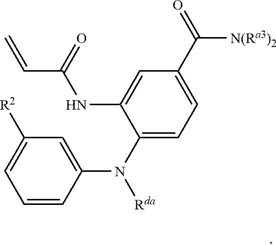

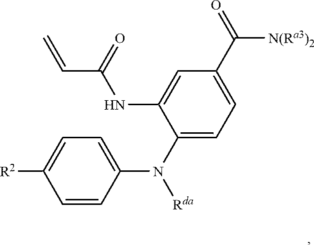

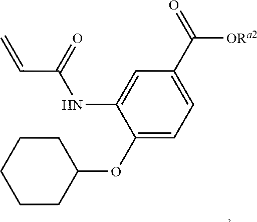

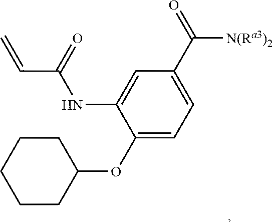

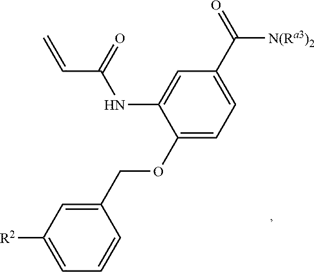

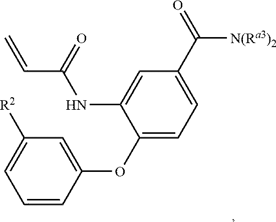

or a pharmaceutically acceptable salt, solvate, hydrate, polymorph, co-crystal, tautomer, stereoisomer, isotopically labeled derivative, or prodrug thereof.

In certain embodiments, the compound of Formula (I-B) is of formula:

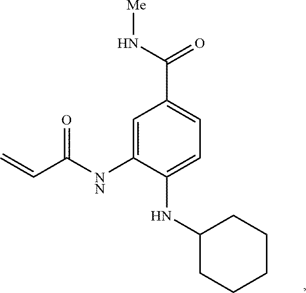

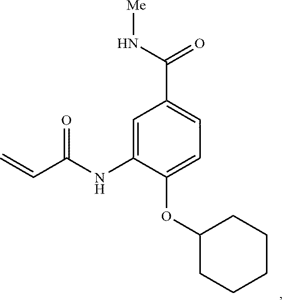

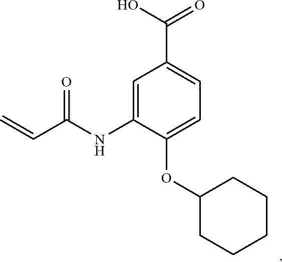

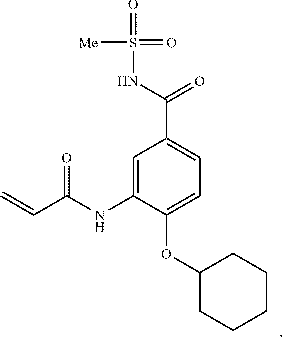

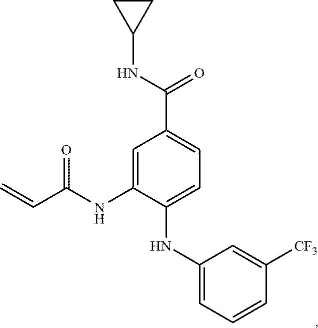

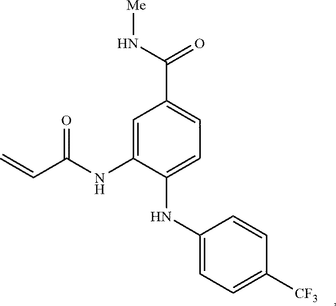

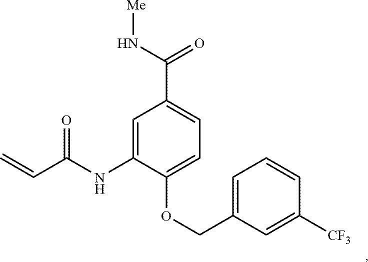

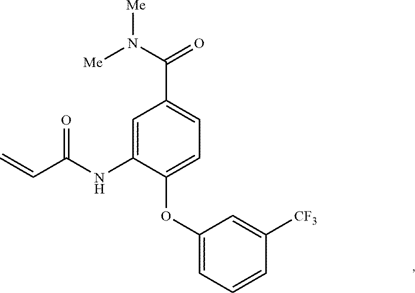

or a pharmaceutically acceptable salt, co-crystal, tautomer, stereoisomer, solvate, hydrate, polymorph, isotopically enriched derivative, or prodrug thereof.

In certain embodiments, the compound of Formula (I-B) is of formula:

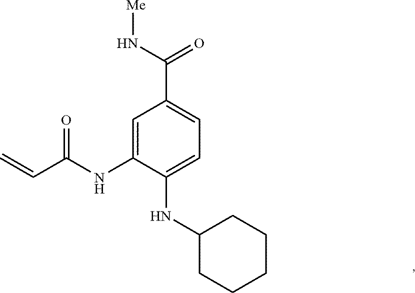

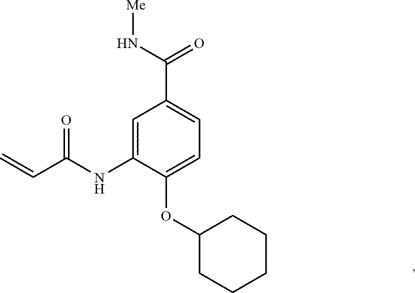

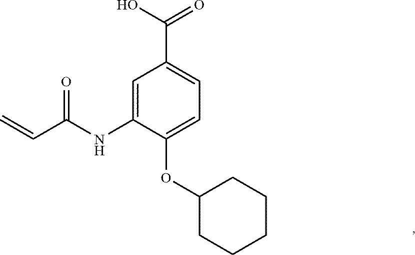

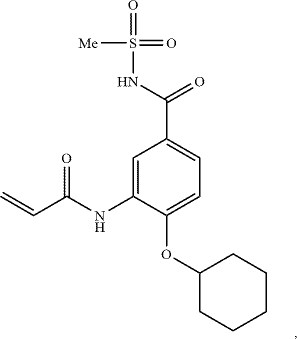

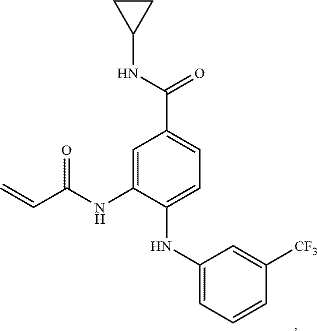

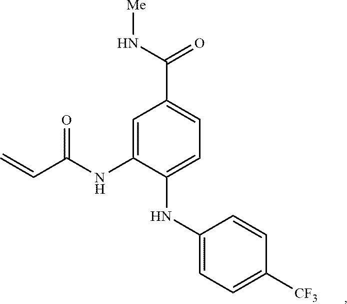

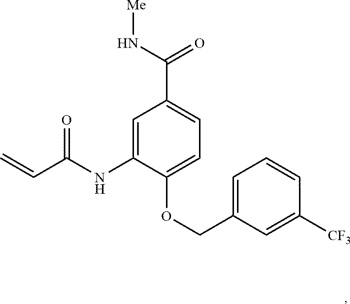

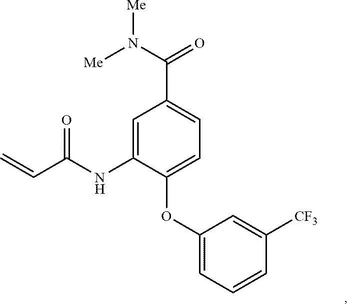

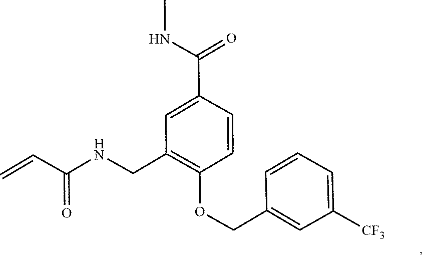

or a pharmaceutically acceptable salt, co-crystal, tautomer, stereoisomer, solvate, hydrate, polymorph, isotopically enriched derivative, or prodrug thereof.

In certain embodiments, the compound of Formula (I-B) is of formula:

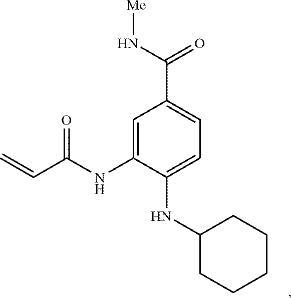

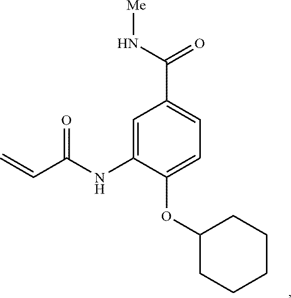

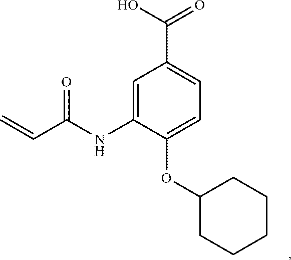

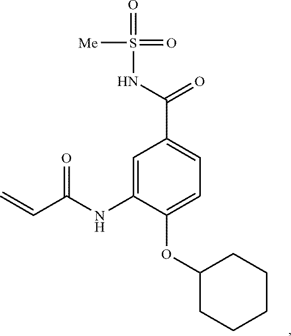

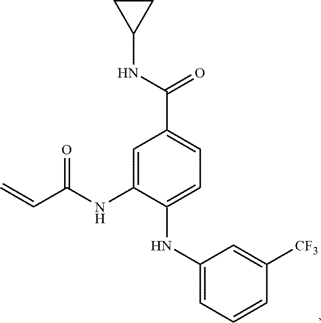

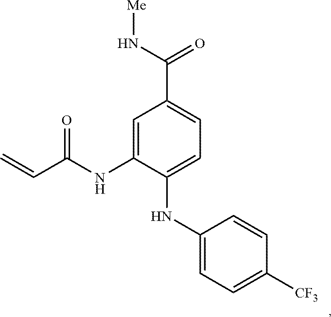

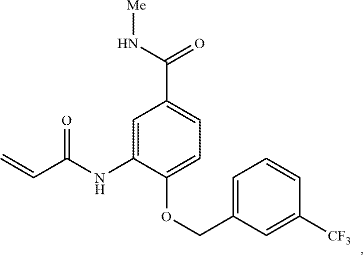

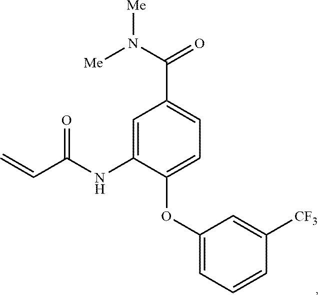

or a pharmaceutically acceptable salt thereof.

In certain embodiments, the compound of Formula (I-B) is of formula:

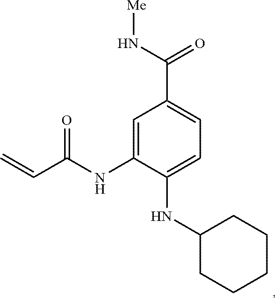

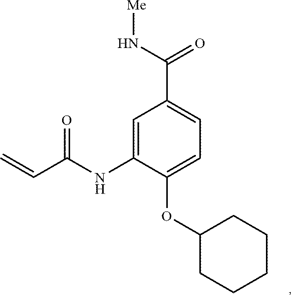

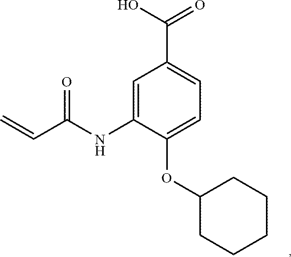

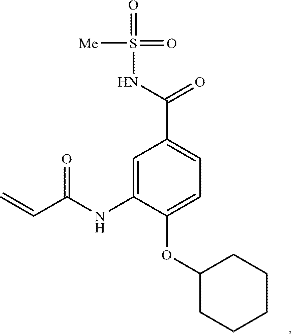

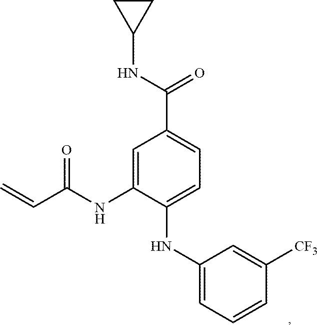

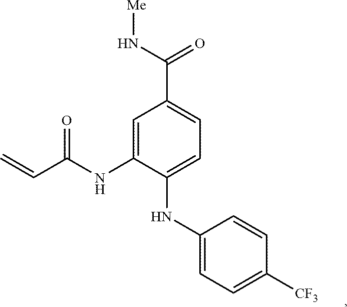

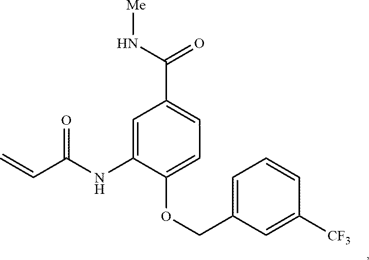

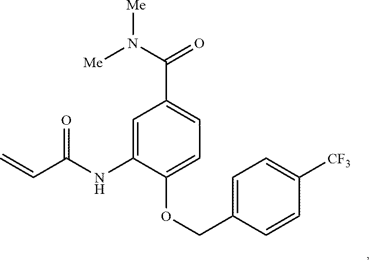

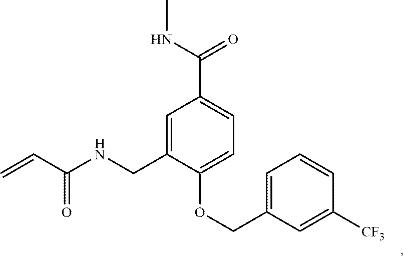

or a pharmaceutically acceptable salt thereof.

In certain embodiments, the compound of Formula (I-A) or (I-B) is of formula:

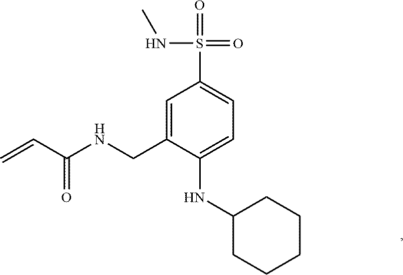

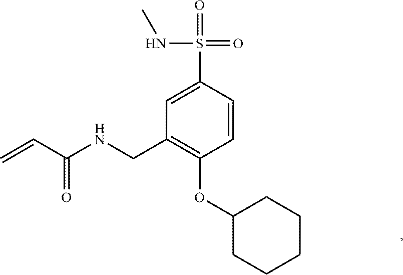

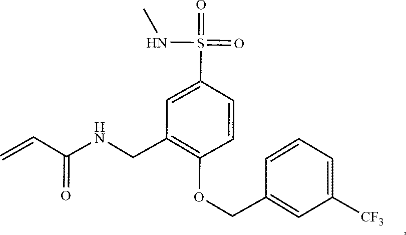

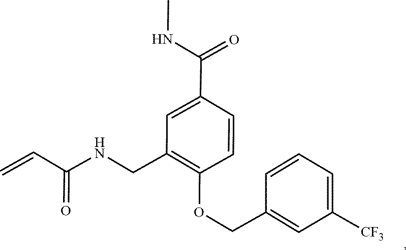

or a pharmaceutically acceptable salt, co-crystal, tautomer, stereoisomer, solvate, hydrate, polymorph, isotopically enriched derivative, or prodrug thereof.

In certain embodiments, the compound of Formula (I-A) or (I-B) is of formula:

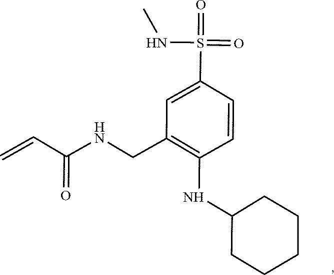

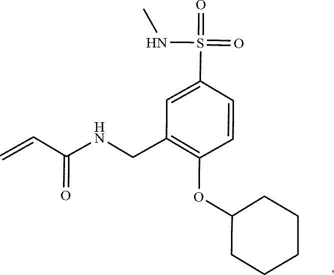

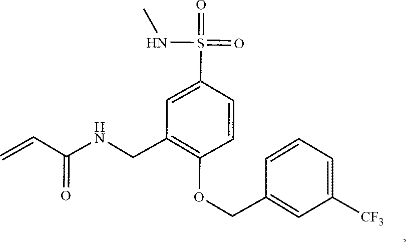

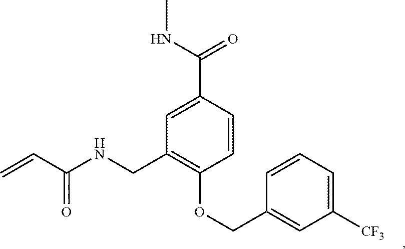

or a pharmaceutically acceptable salt thereof.

In certain embodiments, the compound of Formula (I-B) is not of formula:

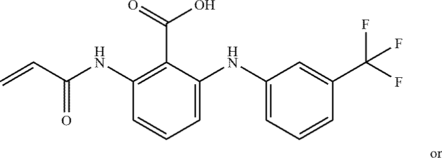

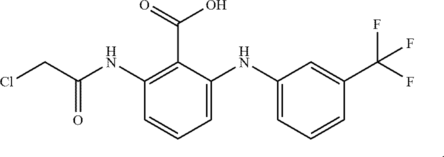

In certain embodiments, the compound of Formula (I-B) is not a compound disclosed in PCT Application no. PCT/US2019/056347, filed Oct. 15, 2019; PCT Application Publication no. WO 2018/204532, published Nov. 8, 2018, or PCT Application Publication No. WO 2019/113236, published Jun. 13, 2019. In certain embodiments, the compound of Formula (I-B) is not a compound disclosed in PCT Application No. PCT/US2019/056347, filed Oct. 15, 2019, and published as PCT Application Publication No. WO2020/081572 on Apr. 23, 2020 (e.g., the compound of Formula (I-B) is not a compound of claim 183 or of Examples 1-6 in PCT/US2019/056347 and WO2020/081572; structures of which are shown above); PCT Application Publication No. WO 2018/204532 (e.g., the compound of Formula (I-B) is not a compound of Tables 1-3, Examples 1-252 or A1 of WO 2018/204532; structures of which are shown above), published Nov. 8, 2018; or PCT Application Publication No. WO 2019/113236 (e.g., the compound of Formula (I-B) is not a compound of Table 1, Examples 1-56 or A1 of WO 2019/113236:

| Compound No. | Structure |
|---|---|
| 1 | 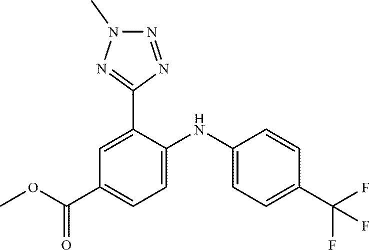 [SMILES: COC(=O)c1ccc(Nc2ccc(C(F)(F)F)cc2)c(-c2nnn(C)n2)c1] |
| 2 | 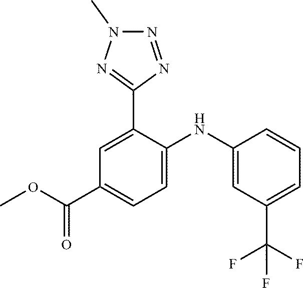 [SMILES: COC(=O)c1ccc(Nc2cccc(C(F)(F)F)c2)c(-c2nnn(C)n2)c1] |
| 3 | 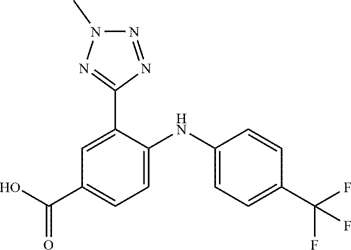 [SMILES: Cn1nnc(-c2cc(C(=O)O)ccc2Nc2ccc(C(F)(F)F)cc2)n1] |
| 4 | 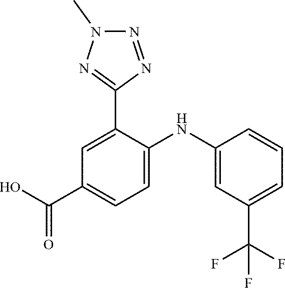 [SMILES: Cn1nnc(-c2cc(C(=O)O)ccc2Nc2cccc(C(F)(F)F)c2)n1] |
| 5 | 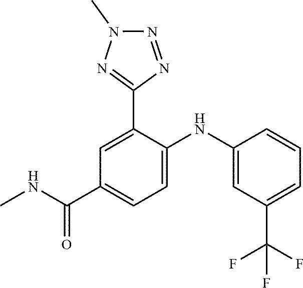 [SMILES: CNC(=O)c1ccc(Nc2cccc(C(F)(F)F)c2)c(-c2nnn(C)n2)c1] |

| | |
|---|---|
| 6 | 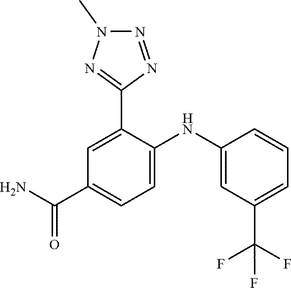 [SMILES: Cn1nnc(-c2cc(C(N)=O)ccc2Nc2cccc(C(F)(F)F)c2)n1] |
| 7 | 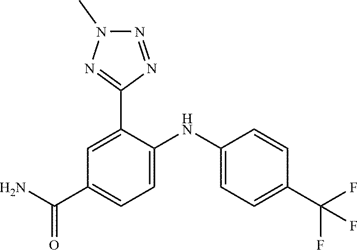 [SMILES: Cn1nnc(-c2cc(C(N)=O)ccc2Nc2ccc(C(F)(F)F)cc2)n1] |
| 8 | 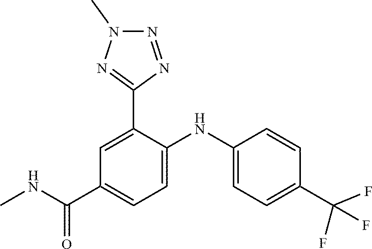 [SMILES: CNC(=O)c1ccc(Nc2ccc(C(F)(F)F)cc2)c(-c2nnn(C)n2)c1] |
| 9 | 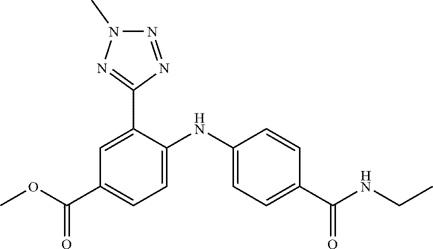 [SMILES: CCNC(=O)c1ccc(Nc2ccc(C(=O)OC)cc2-c2nnn(C)n2)cc1] |
| 10 | 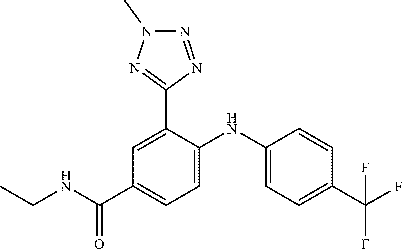 [SMILES: CCNC(=O)c1ccc(Nc2ccc(C(F)(F)F)cc2)c(-c2nnn(C)n2)c1] |
| 11 | 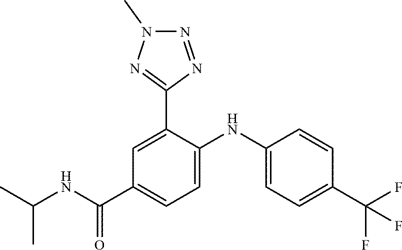 [SMILES: CC(C)NC(=O)c1ccc(Nc2ccc(C(F)(F)F)cc2)c(-c2nnn(C)n2)c1] |

| | |
|---|---|
| 12 | 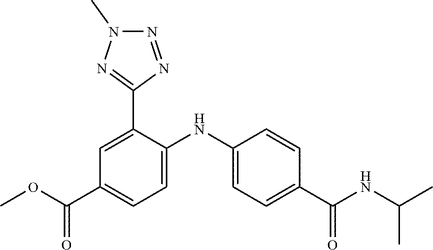 [SMILES: COC(=O)c1ccc(Nc2ccc(C(=O)NC(C)C)cc2)c(-c2nnn(C)n2)c1] |
| 13 | 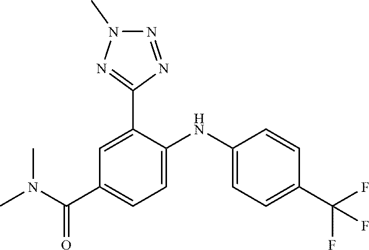 [SMILES: CN(C)C(=O)c1ccc(Nc2ccc(C(F)(F)F)cc2)c(-c2nnn(C)n2)c1] |
| 14 | 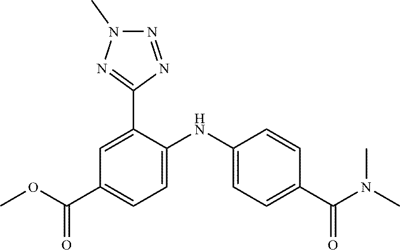 |
| 15 | 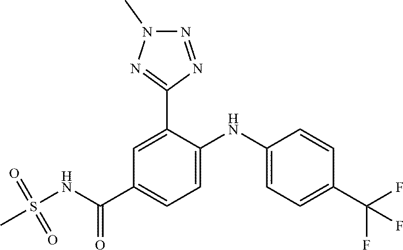 [SMILES: Cn1nnc(-c2cc(C(=O)NS(C)(=O)=O)ccc2Nc2ccc(C(F)(F)F)cc2)n1] |
| 16 | 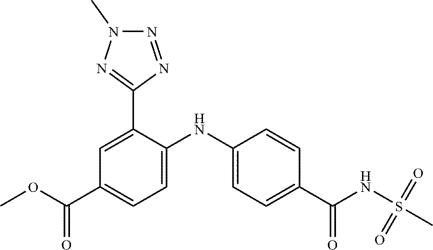 |
| 17 | 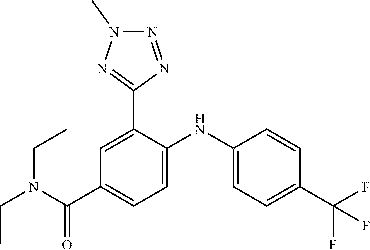 [SMILES: CCN(CC)C(=O)c1ccc(Nc2ccc(C(F)(F)F)cc2)c(-c2nnn(C)n2)c1] |

| | |
|---|---|
| 18 | 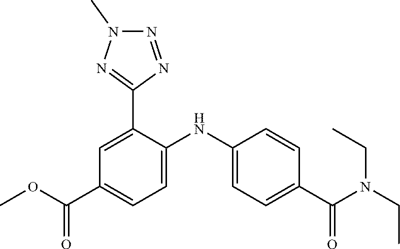 [SMILES: CCN(CC)C(=O)c1ccc(Nc2ccc(C(=O)OC)cc2-c2nnn(C)n2)cc1] |
| 19 | 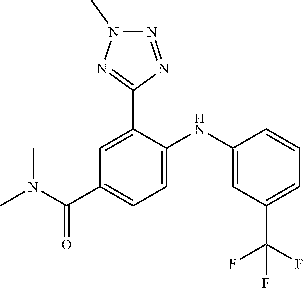 [SMILES: CN(C)C(=O)c1ccc(Nc2cccc(C(F)(F)F)c2)c(-c2nnn(C)n2)c1] |
| 20 | 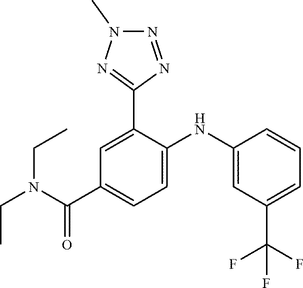 [SMILES: CCN(CC)C(=O)c1ccc(Nc2cccc(C(F)(F)F)c2)c(-c2nnn(C)n2)c1] |
| 21 | 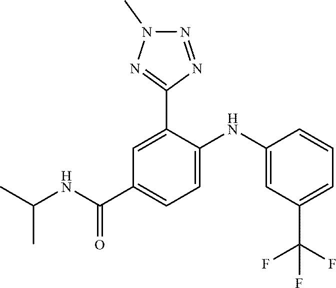 [SMILES: CC(C)NC(=O)c1ccc(Nc2cccc(C(F)(F)F)c2)c(-c2nnn(C)n2)c1] |
| 22 | 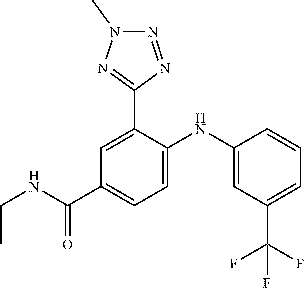 [SMILES: CCNC(=O)c1ccc(Nc2cccc(C(F)(F)F)c2)c(-c2nnn(C)n2)c1] |

| | |
|---|---|
| 23 | 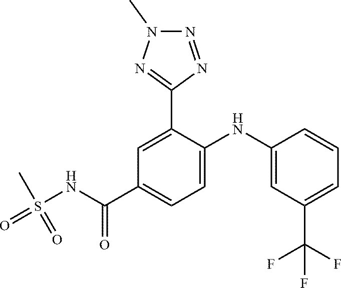 [SMILES: Cn1nnc(-c2cc(C(=O)NS(C)(=O)=O)ccc2Nc2cccc(C(F)(F)F)c2)n1] |
| 24 | 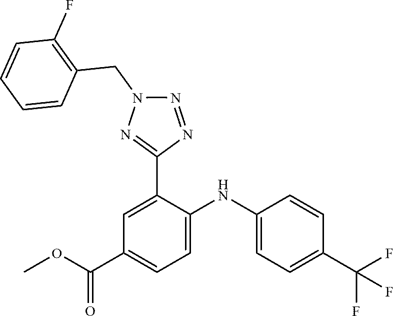 [SMILES: COC(=O)c1ccc(Nc2ccc(C(F)(F)F)cc2)c(-c2nnn(Cc3ccccc3F)n2)c1] |
| 25 | 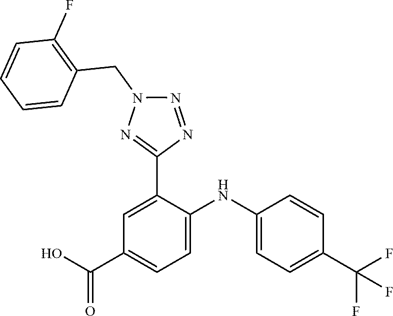 [SMILES: O=C(O)c1ccc(Nc2ccc(C(F)(F)F)cc2)c(-c2nnn(Cc3ccccc3F)n2)c1] |
| 26 | 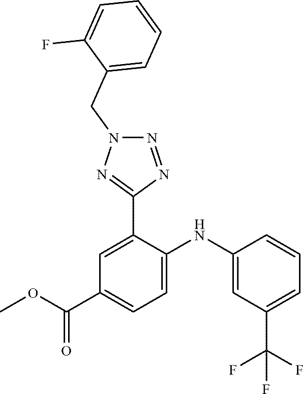 [SMILES: COC(=O)c1ccc(Nc2cccc(C(F)(F)F)c2)c(-c2nnn(Cc3ccccc3F)n2)c1] |

| | |
|---|---|
| 27 | 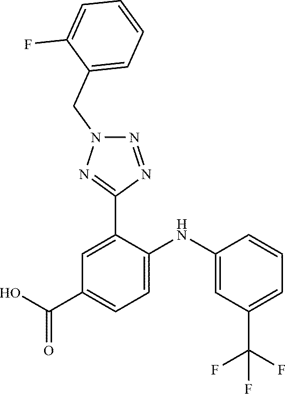 [SMILES: O=C(O)c1ccc(Nc2cccc(C(F)(F)F)c2)c(-c2nnn(Cc3ccccc3F)n2)c1] |
| 28 | 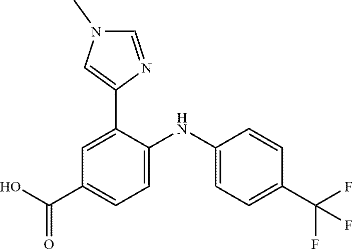 [SMILES: Cn1cnc(-c2cc(C(=O)O)ccc2Nc2ccc(C(F)(F)F)cc2)c1] |
| 29 | 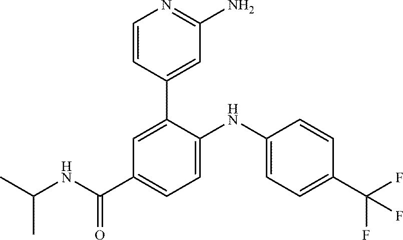 [SMILES: CC(C)NC(=O)c1ccc(Nc2ccc(C(F)(F)F)cc2)c(-c2ccnc(N)c2)c1] |
| 30 | 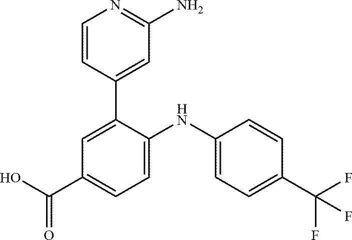 [SMILES: Nc1cc(-c2cc(C(=O)O)ccc2Nc2ccc(C(F)(F)F)cc2)ccn1] |
| 31 | 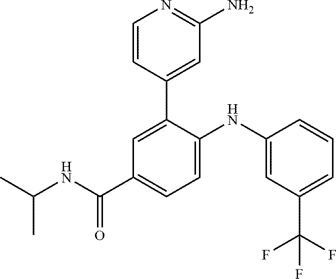 [SMILES: CC(C)NC(=O)c1ccc(Nc2cccc(C(F)(F)F)c2)c(-c2ccnc(N)c2)c1] |

| | |
|---|---|
| 32 | 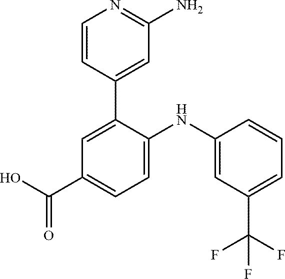 [SMILES: Nc1cc(-c2cc(C(=O)O)ccc2Nc2cccc(C(F)(F)F)c2)ccn1] |
| 33 | 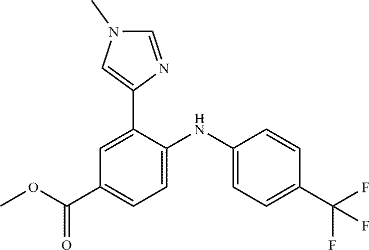 [SMILES: COC(=O)c1ccc(Nc2ccc(C(F)(F)F)cc2)c(-c2cn(C)cn2)c1] |
| 34 | 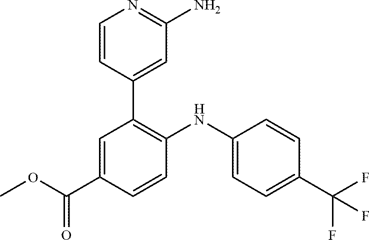 [SMILES: COC(=O)c1ccc(Nc2ccc(C(F)(F)F)cc2)c(-c2ccnc(N)c2)c1] |
| 35 | 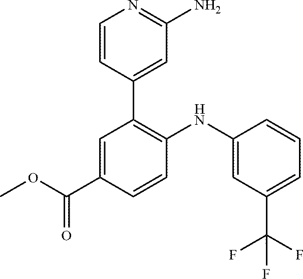 [SMILES: COC(=O)c1ccc(Nc2cccc(C(F)(F)F)c2)c(-c2ccnc(N)c2)c1] |
| 36 | 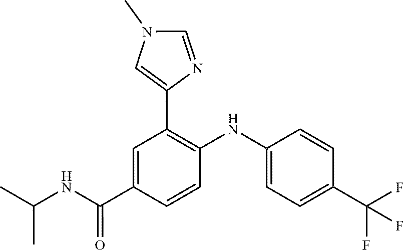 [SMILES: CC(C)NC(=O)c1ccc(Nc2ccc(C(F)(F)F)cc2)c(-c2cn(C)cn2)c1] |
| 37 | 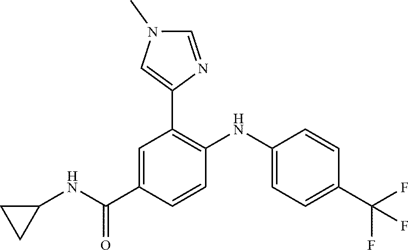 [SMILES: Cn1cnc(-c2cc(C(=O)NC3CC3)ccc2Nc2ccc(C(F)(F)F)cc2)c1] |

| | |
|---|---|
| 38 | 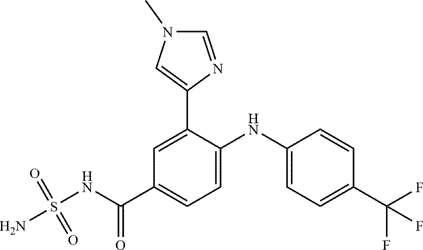 [SMILES: Cn1cnc(-c2cc(C(=O)NS(N)(=O)=O)ccc2Nc2ccc(C(F)(F)F)cc2)c1] |
| 39 | 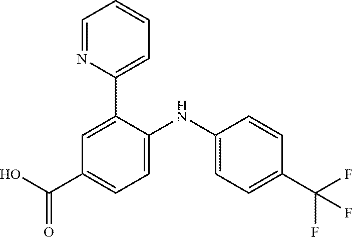 [SMILES: O=C(O)c1ccc(Nc2ccc(C(F)(F)F)cc2)c(-c2ccccn2)c1] |
| 40 | 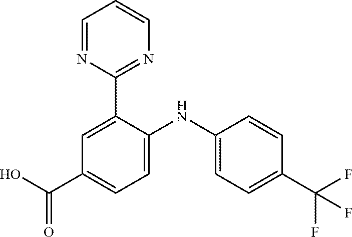 [SMILES: O=C(O)c1ccc(Nc2ccc(C(F)(F)F)cc2)c(-c2ncccn2)c1] |
| 41 | 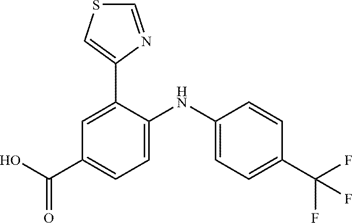 [SMILES: O=C(O)c1ccc(Nc2ccc(C(F)(F)F)cc2)c(-c2cscn2)c1] |
| 42 | 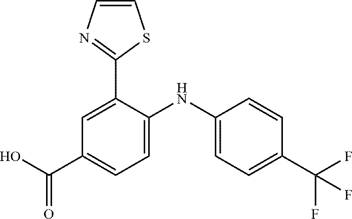 [SMILES: O=C(O)c1ccc(Nc2ccc(C(F)(F)F)cc2)c(-c2nccs2)c1] |
| 43 | 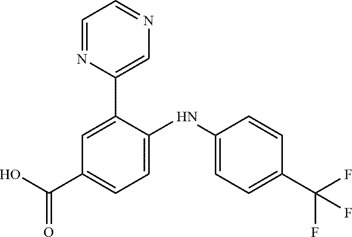 [SMILES: O=C(O)c1ccc(Nc2ccc(C(F)(F)F)cc2)c(-c2cnccn2)c1] |
| 44 | 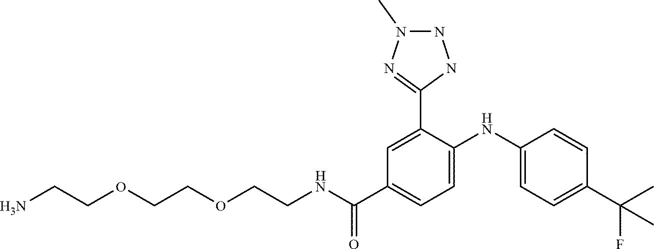 [SMILES: CN1N=C(c2cc(C(=O)NCCOCCOCCN)ccc2Nc2ccc(C(C)(C)F)cc2)NN1] |

| | |
|---|---|
| 45 | 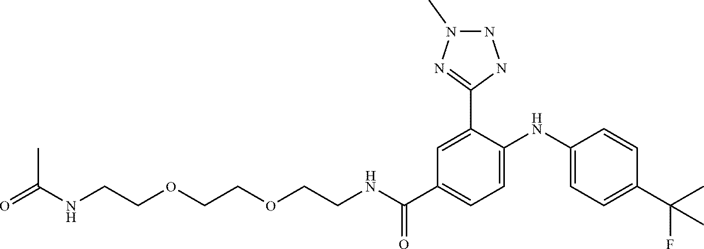 [SMILES: CC(=O)NCCOCCOCCNC(=O)c1ccc(Nc2ccc(C(C)(C)F)cc2)c(C2=NN(C)NN2)c1] |
| 46 | 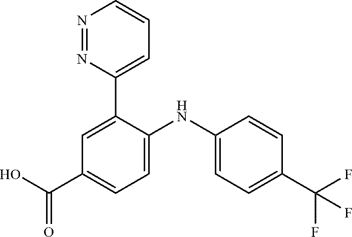 [SMILES: O=C(O)c1ccc(Nc2ccc(C(F)(F)F)cc2)c(-c2cccnn2)c1] |
| 47 | 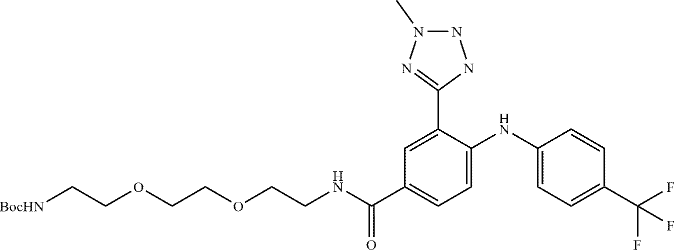 [SMILES: CN1N=C(c2cc(C(=O)NCCOCCOCCNC(=O)OC(C)(C)C)ccc2Nc2ccc(C(F)(F)F)cc2)NN1] |
| 48 | 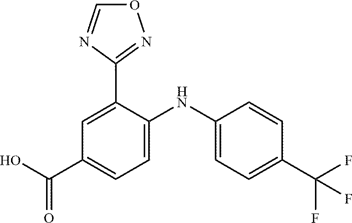 [SMILES: O=C(O)c1ccc(Nc2ccc(C(F)(F)F)cc2)c(-c2ncon2)c1] |
| 49 | 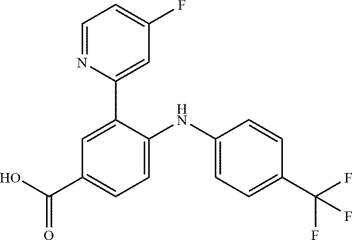 [SMILES: O=C(O)c1ccc(Nc2ccc(C(F)(F)F)cc2)c(-c2cc(F)ccn2)c1] |
| 50 | 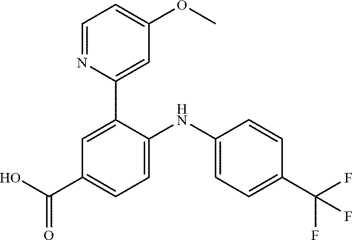 [SMILES: COc1ccnc(-c2cc(C(=O)O)ccc2Nc2ccc(C(F)(F)F)cc2)c1] |
| 51 | 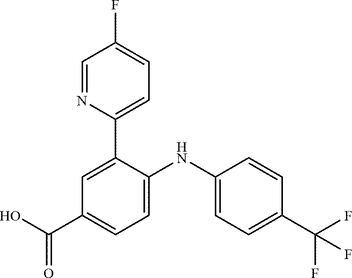 [SMILES: O=C(O)c1ccc(Nc2ccc(C(F)(F)F)cc2)c(-c2ccc(F)cn2)c1] |

| | |
|---|---|
| 52 | 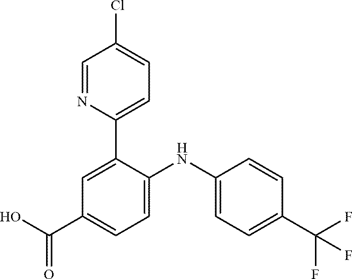 [SMILES: O=C(O)c1ccc(Nc2ccc(C(F)(F)F)cc2)c(-c2ccc(Cl)cn2)c1] |
| 53 | 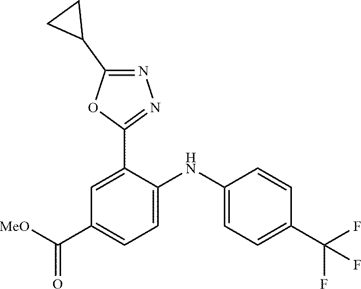 [SMILES: COC(=O)c1ccc(Nc2ccc(C(F)(F)F)cc2)c(-c2nnc(C3CC3)o2)c1] |
| 54 | 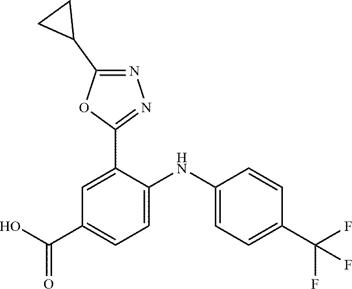 [SMILES: O=C(O)c1ccc(Nc2ccc(C(F)(F)F)cc2)c(-c2nnc(C3CC3)o2)c1] |
| 55 | 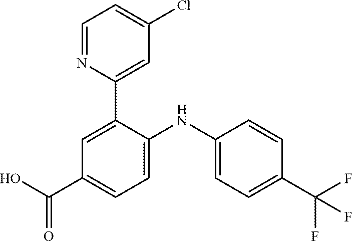 [SMILES: O=C(O)c1ccc(Nc2ccc(C(F)(F)F)cc2)c(-c2cc(Cl)ccn2)c1] |
| 56 | 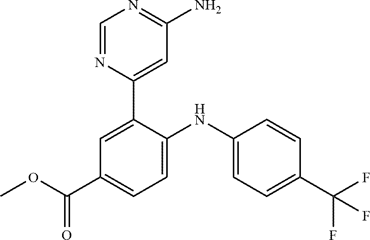 [SMILES: COC(=O)c1ccc(Nc2ccc(C(F)(F)F)cc2)c(-c2cc(N)ncn2)c1] |
| 57 | 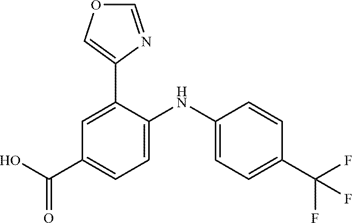 [SMILES: O=C(O)c1ccc(Nc2ccc(C(F)(F)F)cc2)c(-c2cocn2)c1] |

| | |
|---|---|
| 58 | 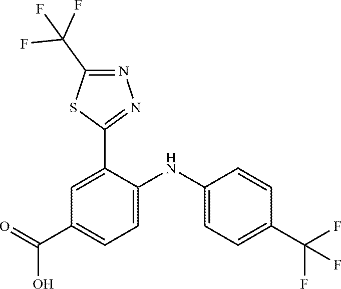 [SMILES: O=C(O)c1ccc(Nc2ccc(C(F)(F)F)cc2)c(-c2nnc(C(F)(F)F)s2)c1] |
| 59 | 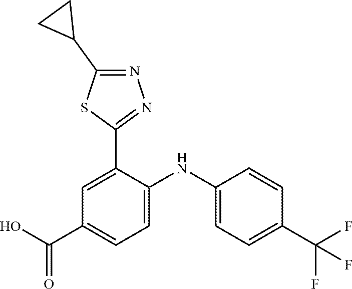 [SMILES: O=C(O)c1ccc(Nc2ccc(C(F)(F)F)cc2)c(-c2nnc(C3CC3)s2)c1] |
| 60 | 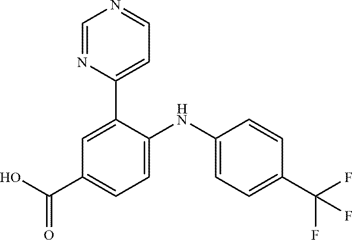 [SMILES: O=C(O)c1ccc(Nc2ccc(C(F)(F)F)cc2)c(-c2ccncn2)c1] |
| 61 | 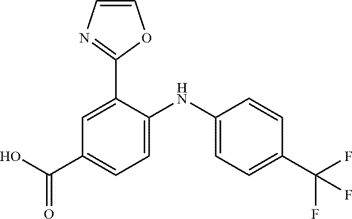 [SMILES: O=C(O)c1ccc(Nc2ccc(C(F)(F)F)cc2)c(-c2ncco2)c1] |
| 62 | 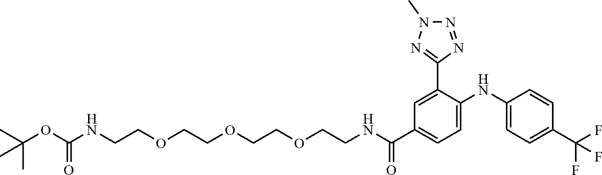 [SMILES: Cn1nnc(-c2cc(C(=O)NCCOCCOCCOCCNC(=O)OC(C)(C)C)ccc2Nc2ccc(C(F)(F)F)cc2)n1] |
| 63 | 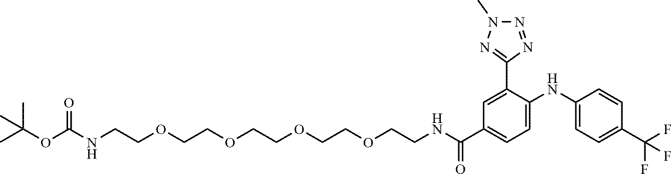 [SMILES: Cn1nnc(-c2cc(C(=O)NCCOCCOCCOCCOCCNC(=O)OC(C)(C)C)ccc2Nc2ccc(C(F)(F)F)cc2)n1] |
| 64 | 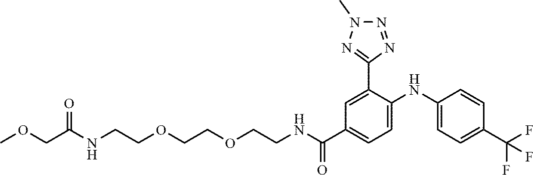 [SMILES: COCC(=O)NCCOCCOCCNC(=O)c1ccc(Nc2ccc(C(F)(F)F)cc2)c(-c2nnn(C)n2)c1] |

| | |
|---|---|
| 65 | 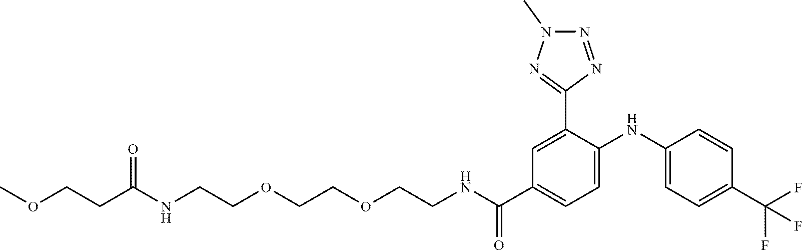 [SMILES: COCCC(=O)NCCOCCOCCNC(=O)c1ccc(Nc2ccc(C(F)(F)F)cc2)c(-c2nnn(C)n2)c1] |

), published Jun. 13, 2019.

### Subgenera of Compounds of Formula (II)

In certain embodiments, a compound of Formula (II) is of formula:

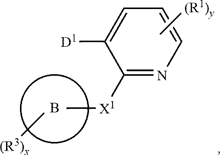

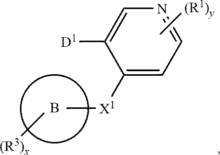

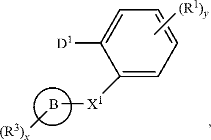

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of formula:

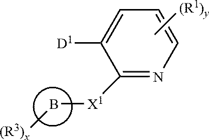

. In certain embodiments, a compound of Formula (II) is of formula:

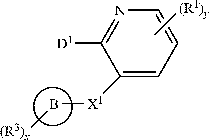

. In certain embodiments, a compound of Formula (II) is of formula:

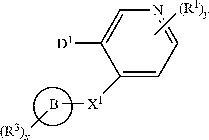

. In certain embodiments, a compound of Formula (II) is of formula:

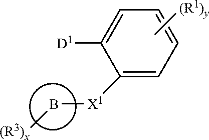

. In certain embodiments, a compound of Formula (II) is of formula:

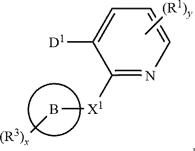

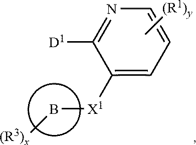

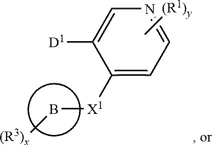

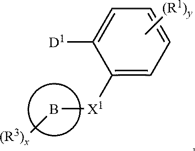

wherein Ring B is cyclohexyl. In certain embodiments, a compound of Formula (II) is of formula:

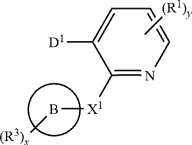

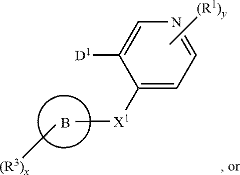

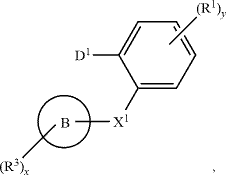

, wherein Ring B is cyclohexyl. In certain embodiments, a compound of Formula (II) is of formula:

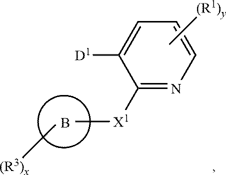

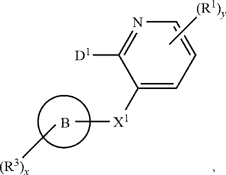

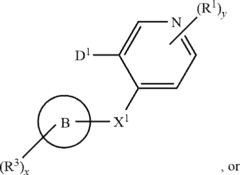

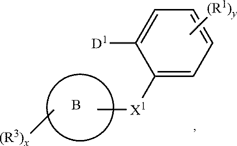

wherein Ring B is phenyl. In certain embodiments, a compound of Formula (II) is of formula:

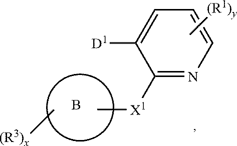

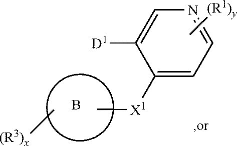

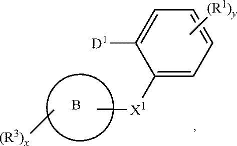

wherein Ring B is phenyl. In certain embodiments, a compound of Formula (II) is of formula:

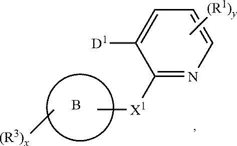

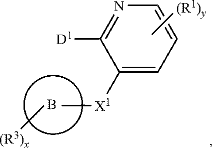

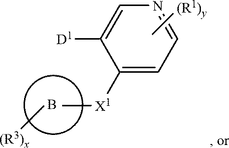

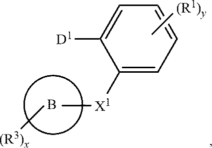

wherein Ring B is cyclohexyl; and y is 0 or 1. In certain embodiments, a compound of Formula (II) is of formula:

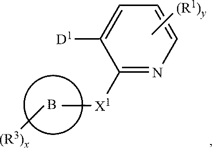

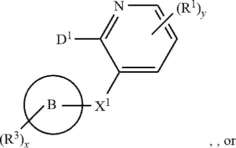

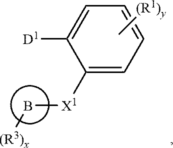

wherein Ring B is phenyl; and y is 0 or 1. In certain embodiments, a compound of Formula (II) is of formula:

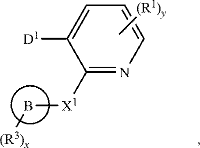

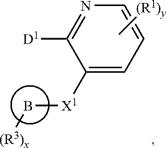

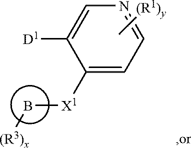

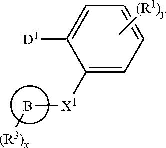

. In certain embodiments, a compound of Formula (II) is of formula:

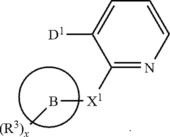

In certain embodiments, a compound of Formula (II) is of formula:

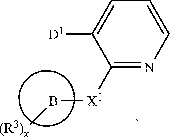

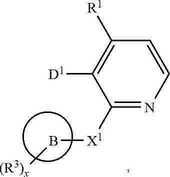

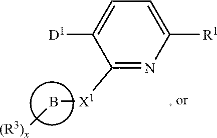

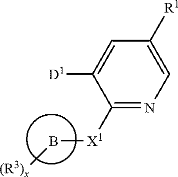

. In certain embodiments, a compound of Formula (II) is of formula:

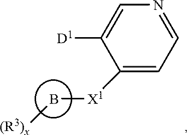

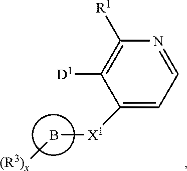

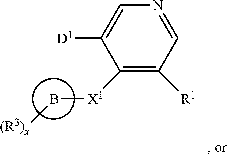

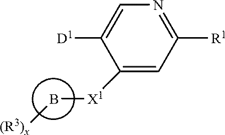

. In certain embodiments, a compound of Formula (II) is of formula: (R³)ₓ

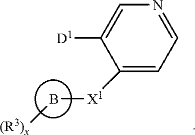

In certain embodiments, a compound of Formula (II) is of formula:

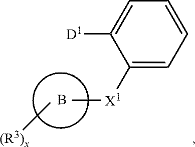

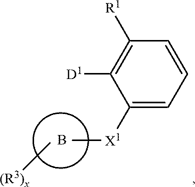

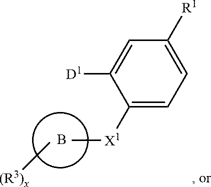

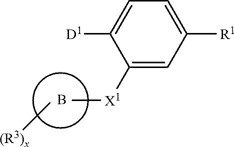

. In certain embodiments, a compound of Formula (II) is of formula:

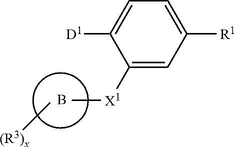

. In certain embodiments, a compound of Formula (II) is of formula:

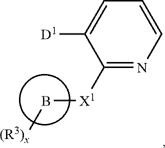

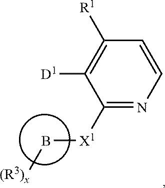

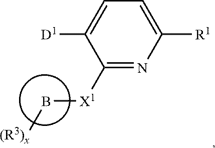

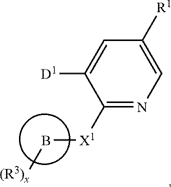

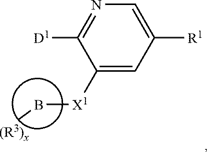

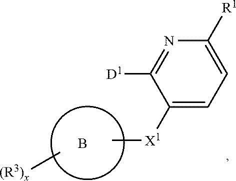

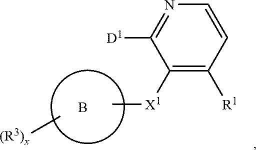

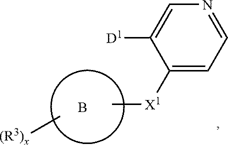

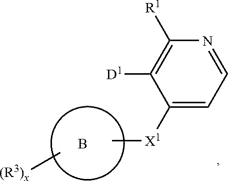

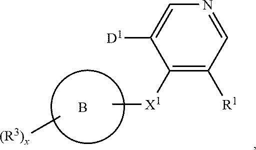

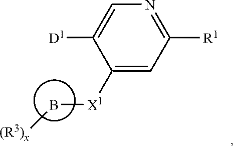

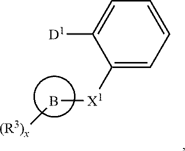

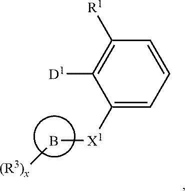

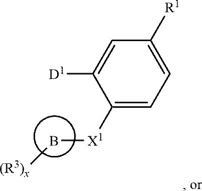

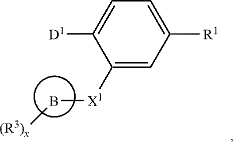

. In certain embodiments, a compound of Formula (II) is of formula:

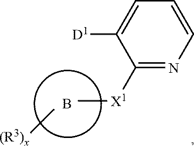

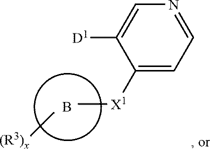

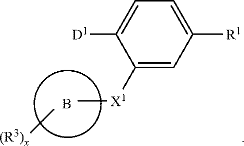

In certain embodiments, a compound of Formula (II) is of formula:

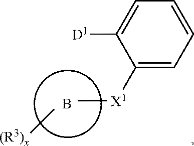

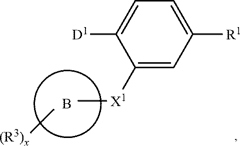

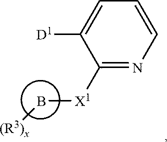

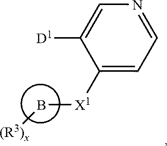

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of formula:

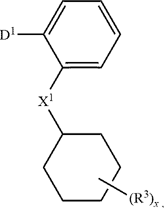

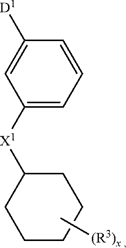

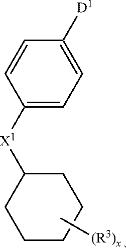

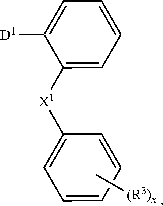

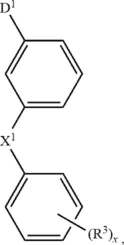

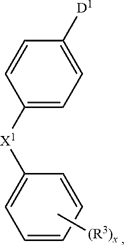

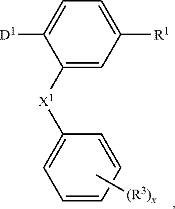

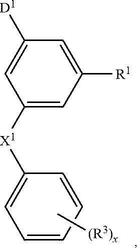

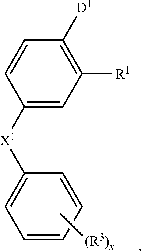

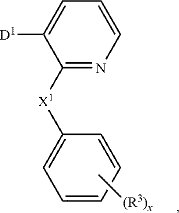

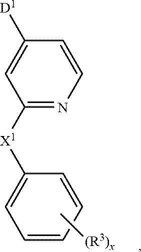

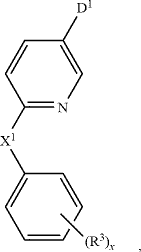

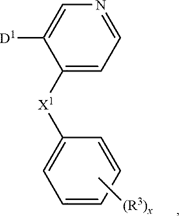

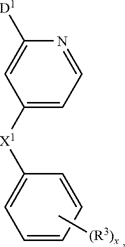

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of formula:

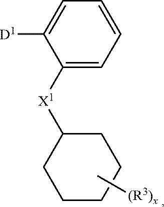

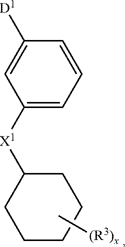

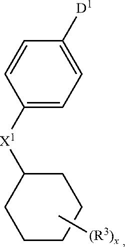

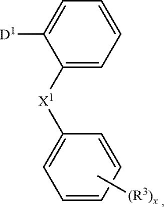

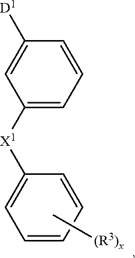

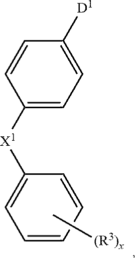

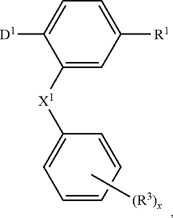

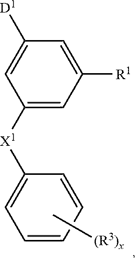

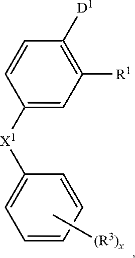

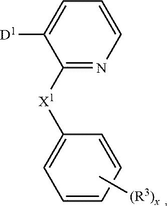

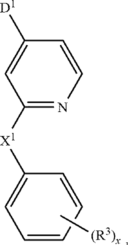

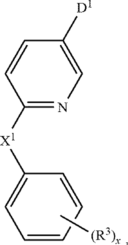

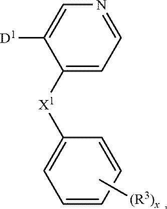

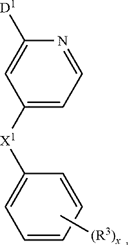

or a pharmaceutically acceptable salt, solvate, hydrate, polymorph, co-crystal, tautomer, stereoisomer, isotopically labeled derivative, or prodrug thereof; and D¹ is a warhead of formula:

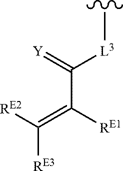

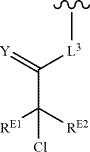

, or

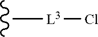

In certain embodiments, a compound of Formula (II) is of formula:

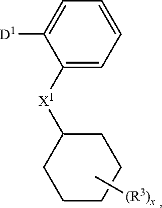

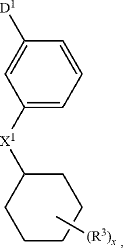

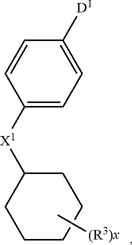

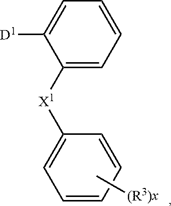

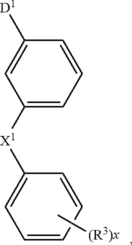

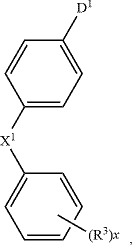

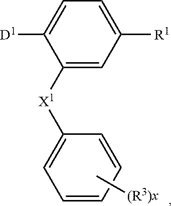

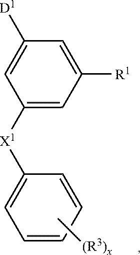

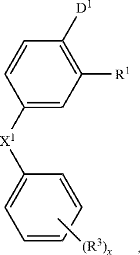

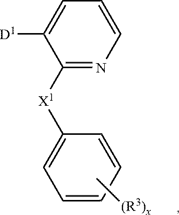

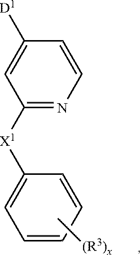

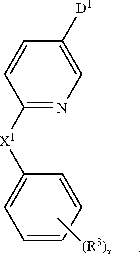

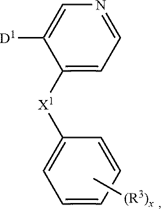

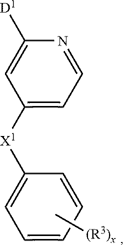

or a pharmaceutically acceptable salt, solvate, hydrate, polymorph, co-crystal, tautomer, stereoisomer, isotopically labeled derivative, or prodrug thereof; and D¹ is a warhead of formula:

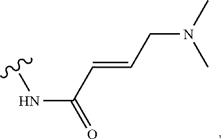

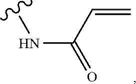

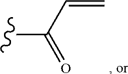

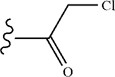

. In certain embodiments, a compound of Formula (II) is of formula:

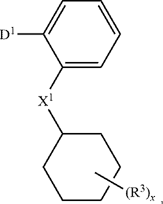

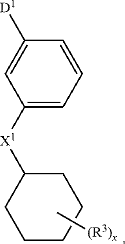

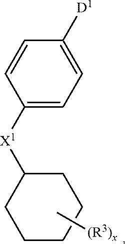

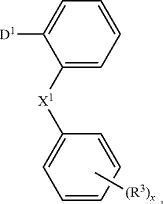

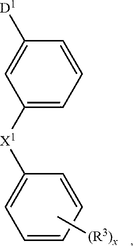

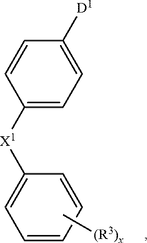

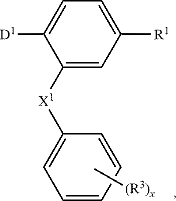

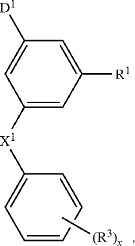

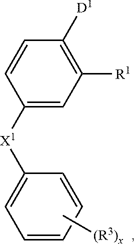

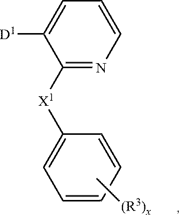

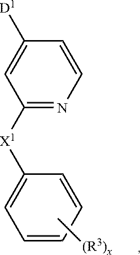

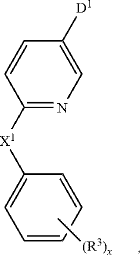

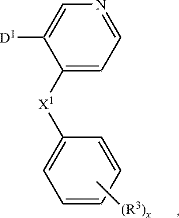

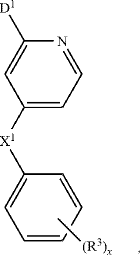

or a pharmaceutically acceptable salt, solvate, hydrate, polymorph, co-crystal, tautomer, stereoisomer, isotopically labeled derivative, or prodrug thereof; and D¹ is a warhead of formula:

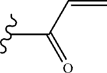

.

In certain embodiments, a compound of Formula (II) is of formula:

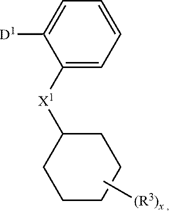

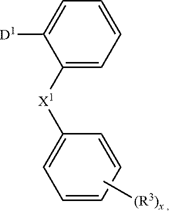

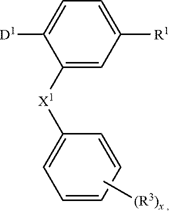

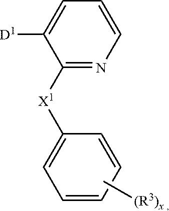

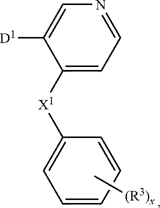

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of formula:

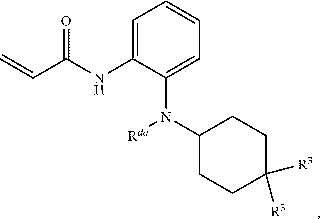

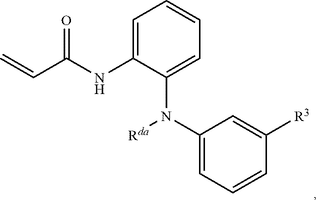

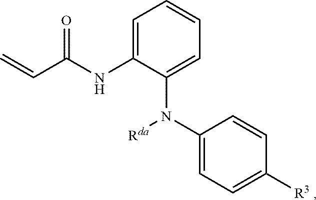

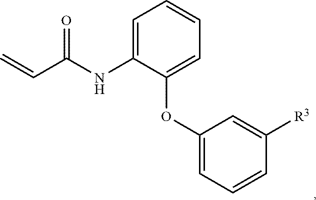

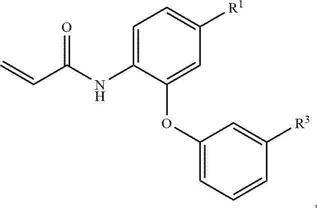

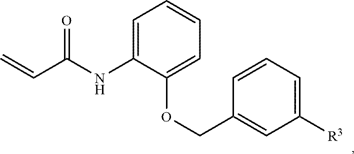

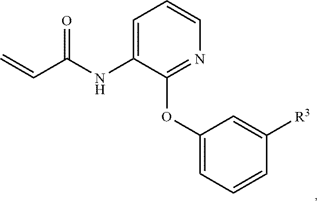

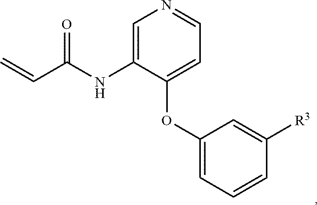

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of formula:

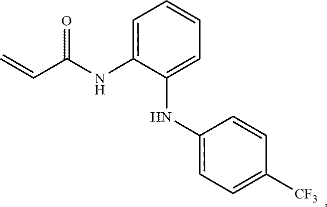

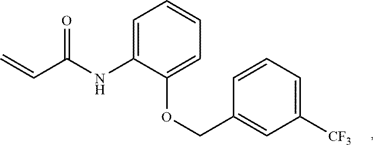

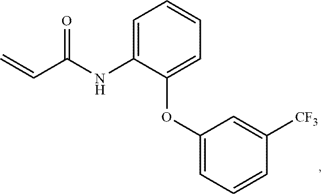

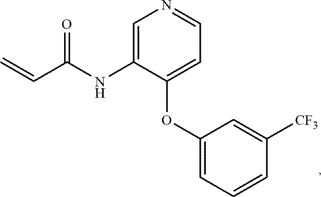

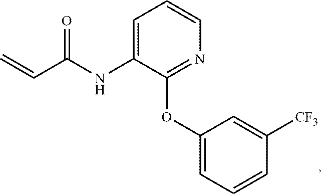

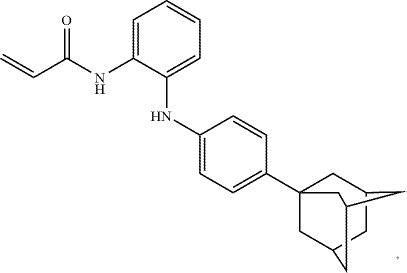

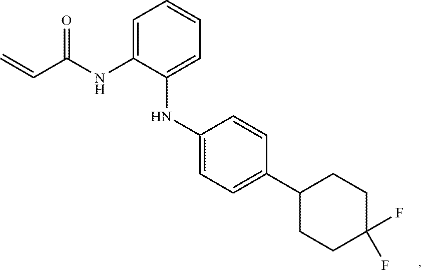

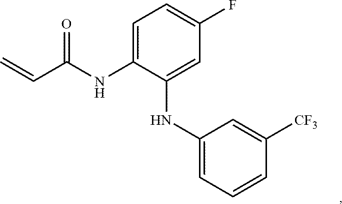

or a pharmaceutically acceptable salt, co-crystal, tautomer, stereoisomer, solvate, hydrate, polymorph, isotopically enriched derivative, or prodrug thereof.

In certain embodiments, a compound of Formula (II) is of formula:

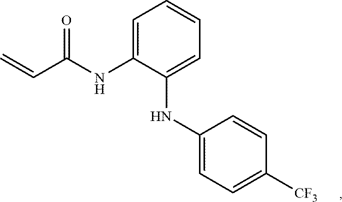

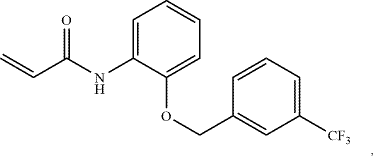

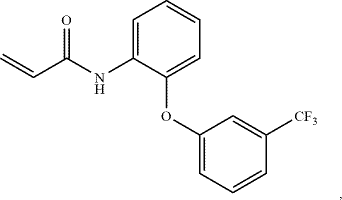

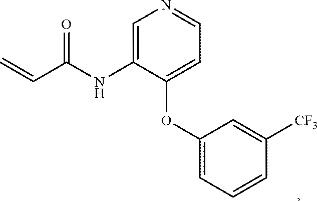

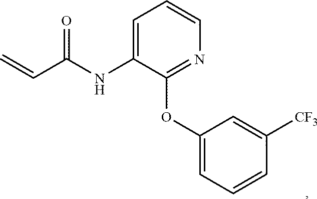

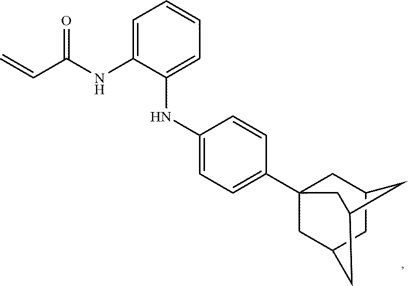

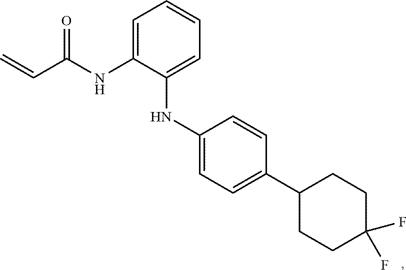

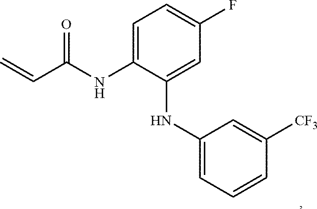

or a pharmaceutically acceptable salt thereof.

In certain embodiments, a compound of Formula (II) is not of formula:

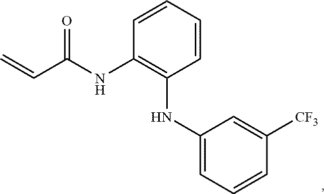

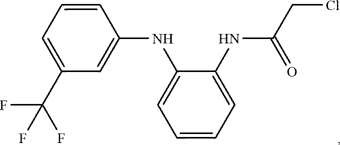

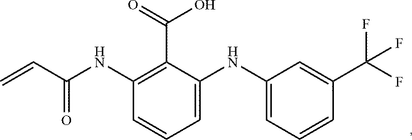

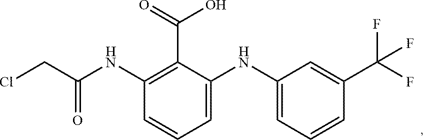

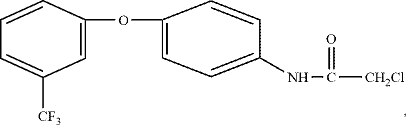

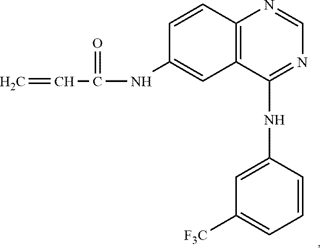

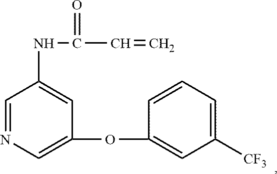

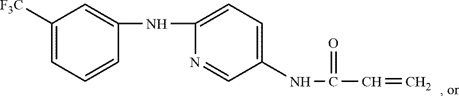

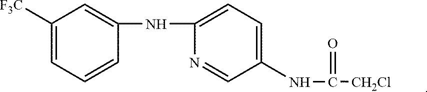

In certain embodiments, the compound of Formula (II) is not a compound disclosed in PCT Application No. PCT/US2019/056347, filed Oct. 15, 2019. In certain embodiments, the compound of Formula (II) is not a compound disclosed in Hamada et al. Yakugaku Zasshi 1980, 100, 829-836), Smaill et al. J. Med. Chem. 1999, 42, 1803-1815, PCT Application Publication No. WO 2017/111076, published Jun. 29, 2017, or PCT Application Publication No. WO 2018/204532, published Nov. 8, 2018. In certain embodiments, the compound of Formula (II) is not a compound disclosed in PCT Application No. PCT/US2019/056347, filed Oct. 15, 2019, and published as PCT Application Publication No. WO2020/081572 on Apr. 23, 2020 (e.g., the compound of Formula (II) is not a compound of claim 183 or of Examples 1-6 in PCT/US2019/056347 and WO2020/081572; structures of which are shown above). In certain embodiments, the compound of Formula (II) is not a compound disclosed in Hamada et al. Yakugaku Zasshi 1980 (e.g., the compound of Formula (II) is not a compound in any of the Figures or schemes of Hamada et al.), 100, 829-836, Smaill et al. J. Med. Chem. 1999 (e.g., the compound of Formula (II) is not a compound in any of the Figures or schemes of Smaill et al.), 42, 1803-1815, PCT Application Publication No. WO 2017/111076, published Jun. 29, 2017 (e.g., the compound of Formula (II) is not a compound of Tables 1-26, Examples 1-142 or A1 of WO 2017/111076:

TABLE1

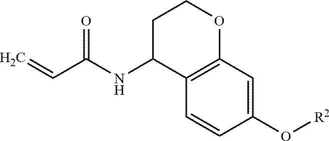

| Compound No. | R² |
|---|---|
| 1 | 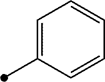 |
| 2 | 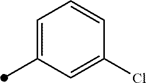 |

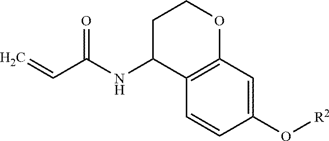

| Compound No. | R² |
|---|---|
| 3 | 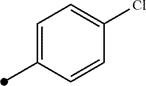 |
| 4 | 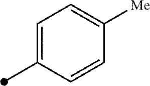 |
| 5 | 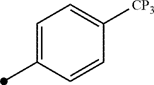 |
| 6 | 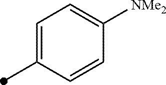 |
| 7 | 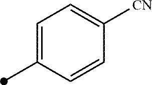 |
| 8 | 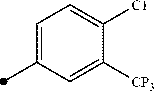 |
| 9 | 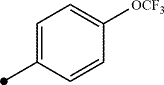 |
| 10 | 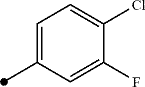 |
| 11 | 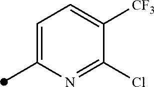 |
| 12 | Compound with retention time of 4.17 minutes among two conditioners contained in compound 3 |
| 13 | Compound with retention time of 3.31 minutes among two conditioners contained in compound 3 |
| 14 | 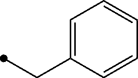 |

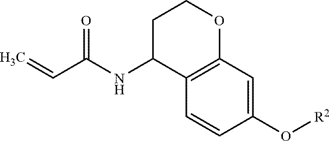

| Compound No. | a² |
|---|---|
| 15 | 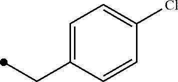 |
| 16 | 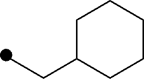 |

TABLE 2

| Com pound No. | |
|---|---|
| 17 | 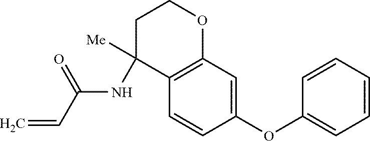 [SMILES: C=CC(=O)NC1(C)CCOc2cc(Oc3ccccc3)ccc21] |
| 18 | 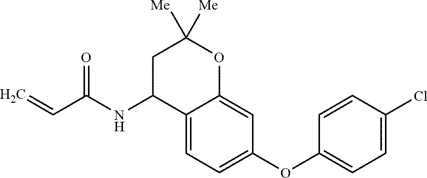 [SMILES: C=CC(=O)NC1CC(C)(C)Oc2cc(Oc3ccc(Cl)cc3)ccc21] |
| 19 | 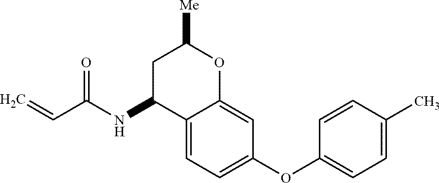 [SMILES: C=CC(=O)N[C@H]1C[C@@H](C)Oc2cc(Oc3ccc(C)cc3)ccc21] |
| 20 | 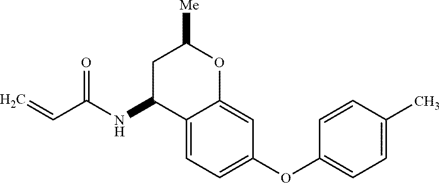 [SMILES: C=CC(=O)N[C@H]1C[C@@H](C)Oc2cc(Oc3ccc(C)cc3)ccc21] |
| 21 | 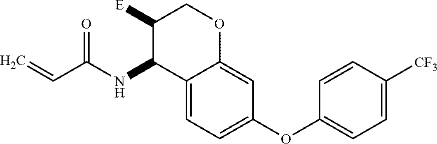 |
| Compound No. | |
| 22 | 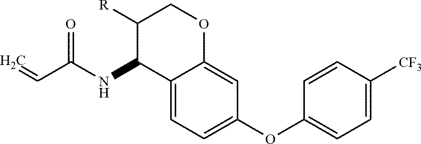 |
| 23 | 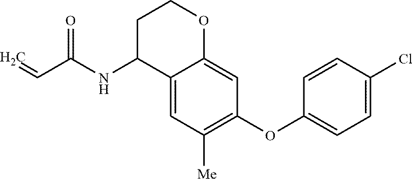 [SMILES: C=CC(=O)NC1CCOc2cc(Oc3ccc(Cl)cc3)c(C)cc21] |
| 24 | 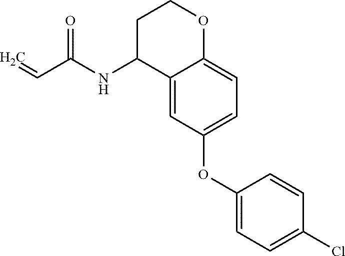 [SMILES: C=CC(=O)NC1CCOc2ccc(Oc3ccc(Cl)cc3)cc21] |
| 25 | 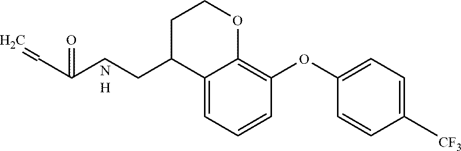 |
| 26 | 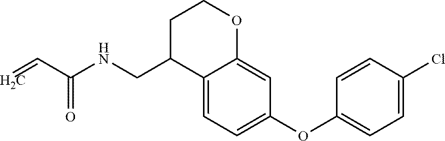 [SMILES: C=CC(=O)NCC1CCOc2cc(Oc3ccc(Cl)cc3)ccc21] |

TABLE 3

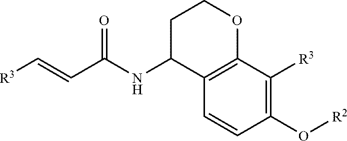

| Compound No. | R¹ | R⁵ | R² |
|---|---|---|---|
| 27 | R | Me | 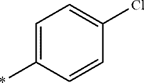 [SMILES: *c1ccc(Cl)cc1] |

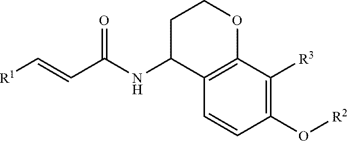

| Compound No. | R¹ | R² | R³ |
|---|---|---|---|
| 28 | H | Me | 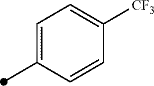 |
| 29 | CP₃ | Me | 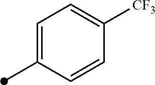 |
| 30 | R | F | 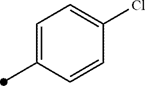 |
| 31 | H | P | 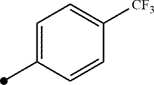 |
| 32 | R | OMe | 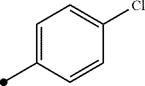 |
| 33 | R | OMe | 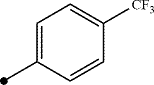 |

TABLE 4

| Compound No. | |
|---|---|
| 34 | 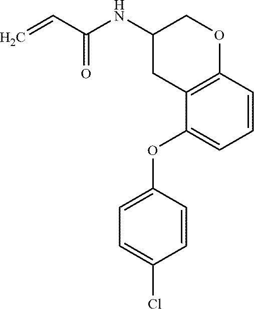 [SMILES: C=CC(=O)NC1COc2cccc(Oc3ccc(Cl)cc3)c2C1] |

| | |
|---|---|
| Compound No. | |
| 35 | 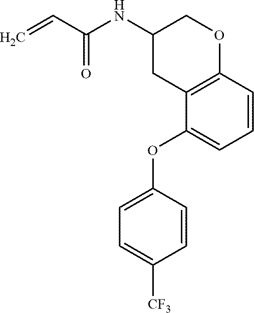 [SMILES: C=CC(=O)NC1COc2cccc(Oc3ccc(C(F)(F)F)cc3)c2C1] |
| 36 | 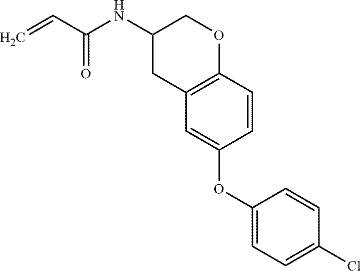 [SMILES: C=CC(=O)NC1COc2ccc(Oc3ccc(Cl)cc3)cc2C1] |
| 37 | 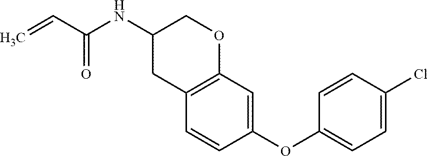 [SMILES: C=CC(=O)NC1COc2cc(Oc3ccc(Cl)cc3)ccc2C1] |
| 38 | 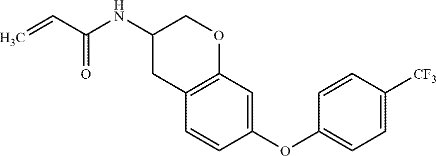 [SMILES: C=CC(=O)NC1COc2cc(Oc3ccc(C(F)(F)F)cc3)ccc2C1] |
| 39 | 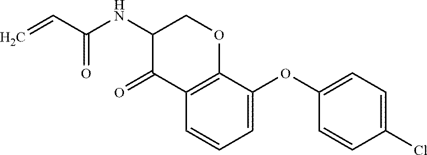 [SMILES: C=CC(=O)NC1COc2c(Oc3ccc(Cl)cc3)cccc2C1=O] |
| 40 | 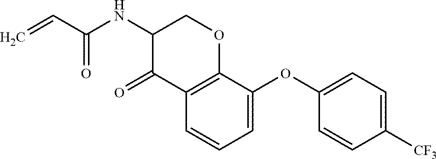 [SMILES: C=CC(=O)NC1COc2c(Oc3ccc(C(F)(F)F)cc3)cccc2C1=O] |

| Compound No. | |
|---|---|
| 41 | 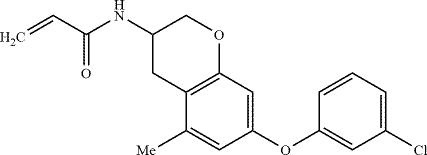 [SMILES: C=CC(=O)NC1COc2cc(Oc3cccc(Cl)c3)cc(C)c2C1] |

TABLE 5

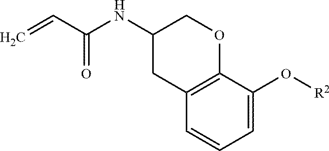

| Compound No. | R² |
|---|---|
| 42 | 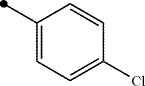 |
| 43 | 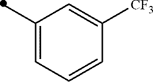 |
| 44 | 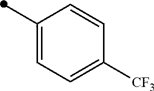 |
| 45 | 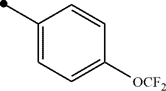 |
| 46 | 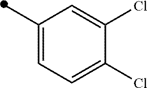 |
| 47 | 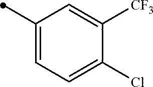 |
| 48 | 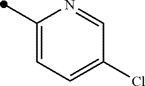 |
| 49 | 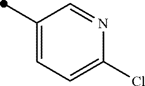 |

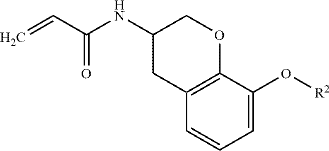

| Compound No. | R² |
|---|---|
| 50 | 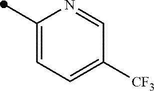 |
| 51 | 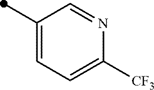 |
| 52 | 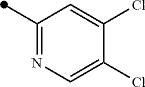 |
| 53 | 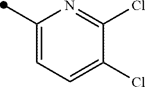 |
| 54 | 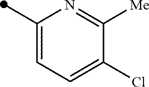 |
| 55 | 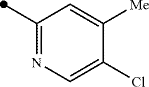 |
| 56 | 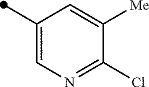 |
| 57 | 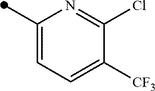 |
| 58 | 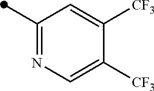 |

TABLE 6

| Compound No. | |
|---|---|
| 59 | Compound with retention time of 3.45 minutes among two substances contained in compound 42 |
| 60 | Compound with retention time of 4.57 minutes among two substances contained in compound 4 3 |
| 61 | Compound with retention time of 4.17 minutes among two chemicals contained in compound 44 |

| Compound No. | |
|---|---|
| 62 | Compound with retention time of 5.74 minutes among two substances contained in compound 44 |
| 63 | Compound with retention time of 5.25 minutes among two substances contained in compound 50 |
| 64 | Compound with retention time of 2.82 minutes among two substances contained in compound 50 |
| 65 | Compound with retention time of 6.65 minutes among two substances contained in compound 35 |
| 66 | Compound with retention time of 8.28 minutes among two substances contained in compound 35 |

TABLE 7

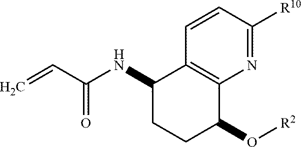

| Compound No. | R¹⁰ | R⁷ |
|---|---|---|
| 67 | H | 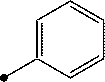 |
| 68 | M | 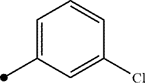 |
| 69 | N | 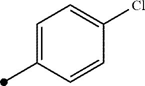 |
| 70 | Cl | 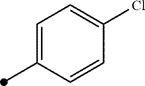 |
| 71 | OMe | 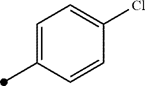 |
| 72 | 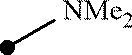 | 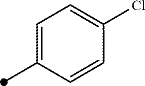 |
| 73 | 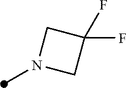 | 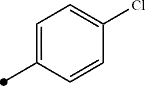 |
| 74 | 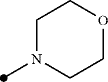 | 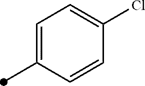 |

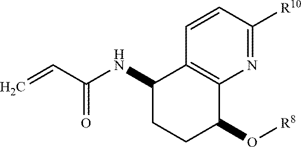

| Compound No. | R³⁰ | R² |
|---|---|---|
| 75 | Cl | 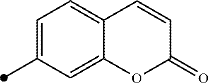 |
| 76 | Cl | 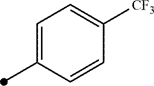 |
| 77 | Cl | 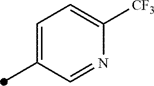 |
| 78 | 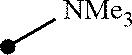 | 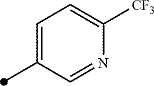 |

TABLE 8

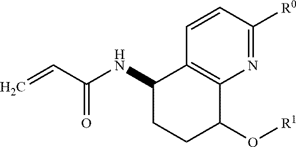

| Compound No. | R¹⁰ | R⁷ |
|---|---|---|
| 79 | H | 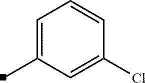 |
| 80 | H | 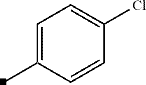 |
| 81 | OMe | 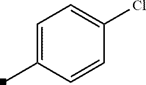 |
| 82 | Cl | 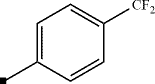 |

TABLE 9

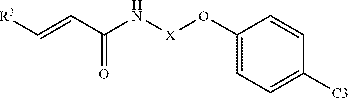

| Compound No. | R¹ | A |
|---|---|---|
| 83 | R | 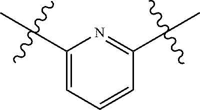 [SMILES: *c1cccc(*)n1] |
| 84 | H | 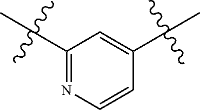 [SMILES: *c1ccnc(*)c1] |
| 85 | H | 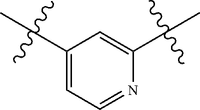 [SMILES: *c1ccnc(*)c1] |
| 86 | H | 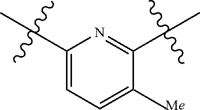 [SMILES: *c1ccc(C)c(*)n1 ; "Me"] |
| 87 | H | 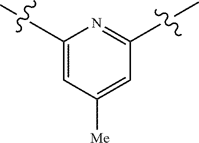 |
| 88 | H | 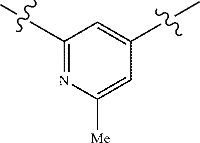 |
| 89 | H | 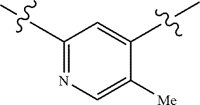 |
| 90 | Me | 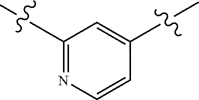 |
| 91 | H | 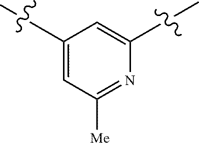 |

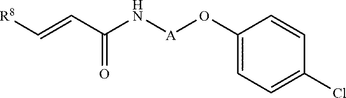

| Compound No. | R¹ | A |
|---|---|---|
| 92 | H | 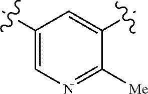 |
| 93 | H | 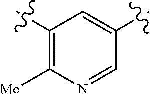 |

TABLE 10

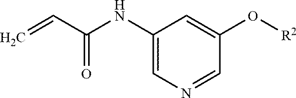

| Compound No | R¹ |
|---|---|
| 94 | 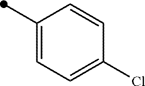 |
| 95 | 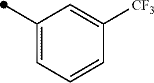 |
| 96 | 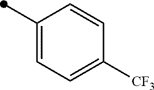 |
| 97 | 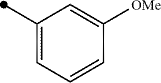 |
| 98 | 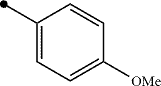 |
| 99 | 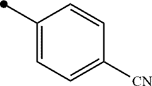 |
| 100 | 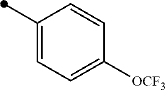 |

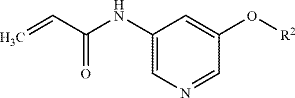

| Compound No. | R² |
|---|---|
| 101 | 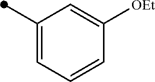 |
| 102 | 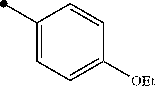 |
| 103 | 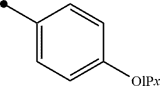 |
| 104 | 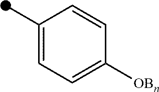 |
| 105 | 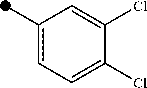 |
| 106 | 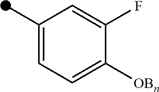 |
| 107 | 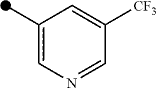 |
| 108 | 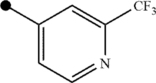 |
| 109 | 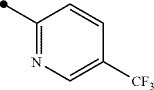 |
| 110 | 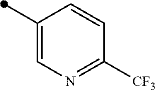 |

TABLE 11

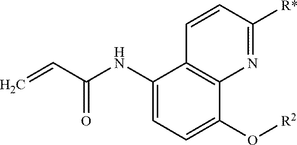

| Compound No. | R¹⁹ | R² |
|---|---|---|
| 111 | H | 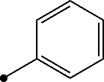 |
| 112 | Me | 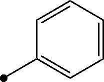 |
| 113 | H | 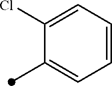 |
| 114 | II | 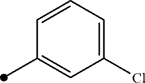 |
| 115 | Me | 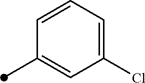 |
| 116 | H | 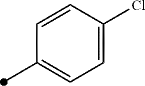 |
| 117 | Me | 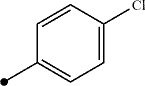 |
| 118 | H | 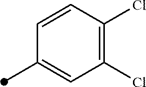 |
| 119 | H | 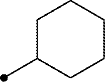 |
| 120 | H | 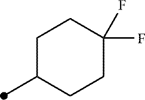 |
| 121 | H | 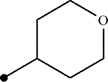 |

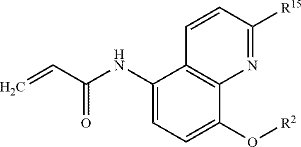

| Compound No. | R¹³ | R² |
|---|---|---|
| 122 | H | 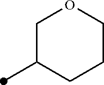 |

TABLE 12

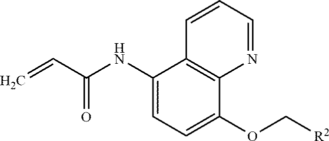

| Compound No. | R⁷ |
|---|---|
| 123 | 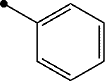 |
| 124 | 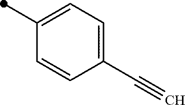 |
| 125 | 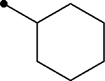 |
| 126 | 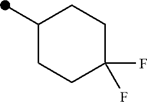 |
| 127 | 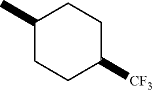 |
| 128 | 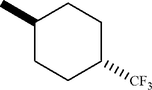 |
| 129 | 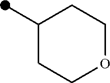 |
| 130 | 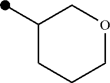 |

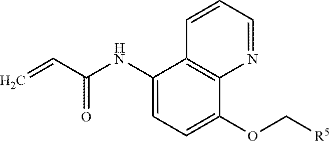

| Compound No. | R² |
|---|---|
| 131 | 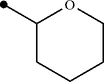 |
| 132 | 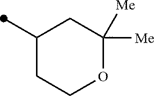 |

TABLE 13

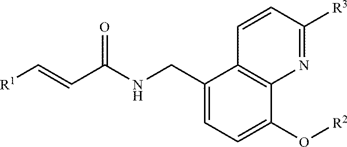

| Compound No. | R¹ | R¹⁵ | R² |
|---|---|---|---|
| 133 | H | H | 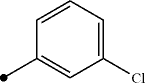 |
| 134 | H | H | 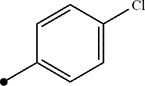 |
| 135 | CF₃ | H | 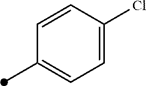 |
| 136 | H | H | 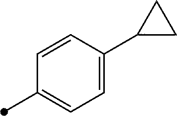 |
| 137 | H | H | 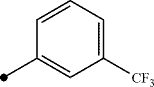 |
| 138 | H | H | 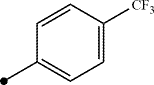 |
| 139 | H | Cl | 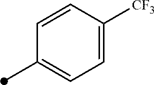 |

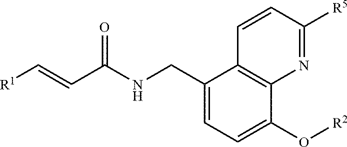

| Compound No. | R¹ | R¹⁸ | R⁸ |
|---|---|---|---|
| 140 | H | H | 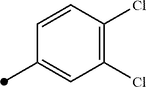 |
| 141 | H | H | 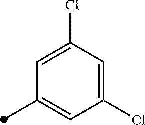 |
| 142 | H | H | 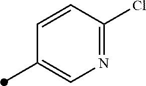 |
| 143 | H | H | 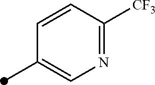 |

TABLE 14

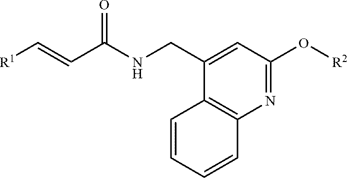

| Compound No. | R¹ | R² |
|---|---|---|
| 144 | H | 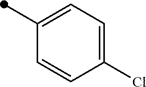 |
| 145 | CF₂ | 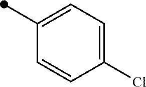 |
| 146 | H | 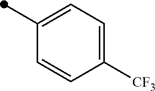 |
| 147 | CF₃ | 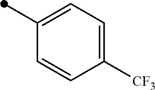 |

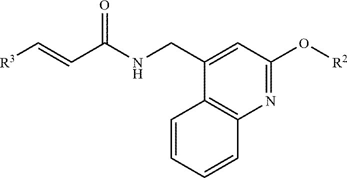

| Compound No. | R¹ | R² |
|---|---|---|
| 148 | H | 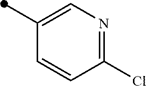 |
| 149 | H | 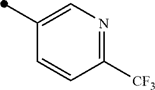 |
| 150 | H | 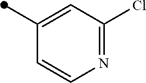 |
| 151 | H | 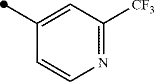 |

TABLE 15

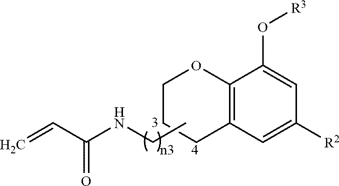

| Compound No. | Substitution position of acrylamide | _{R}1 | R² | R¹ |
|---|---|---|---|---|
| 152 | 4 | 1 | 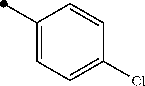 | H |
| 153 | 3 | 0 | 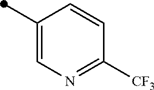 | Rr |
| 154 | 3 | 0 | 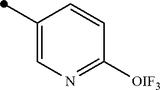 | H |
| 155 | 3 | 0 | 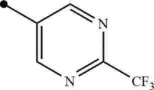 | H |

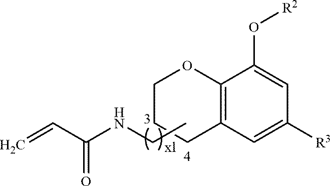

| Compound No. | Subsitution position of acrylamide | _{R}1 | R² | R³ |
|---|---|---|---|---|
| 156 | 3 | 0 | 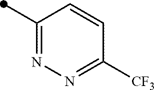 | H |
| 157 | 3 | 0 | 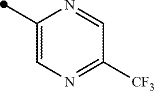 | H |
| 158 | 3 | 0 | 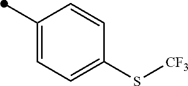 | H |
| 159 | 3 | 0 | 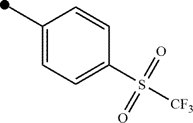 | B |
| 160 | 3 | 0 | 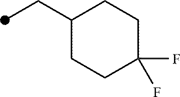 | H |
| 161 | 3 | 0 | 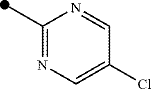 | H |
| 162 | 3 | 0 | 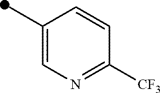 | CF₃ |

TABLE 16

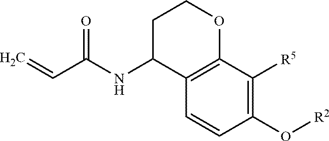

| Compound No. | R² | R⁵ |
|---|---|---|
| 163 | 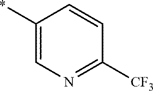 [SMILES: *c1ccc(C(F)(F)F)nc1] | OMe |

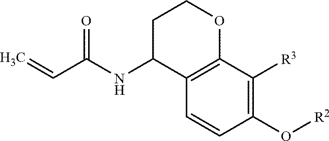

| Compound No. | R² | R³ |
|---|---|---|
| 164 | 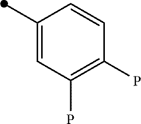 | OMe |
| 165 | 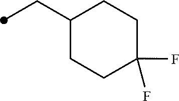 | OMe |
| 166 | 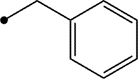 | F |
| 167 | 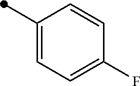 | F |
| 168 | 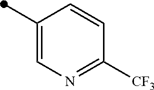 | Cl |
| 169 | 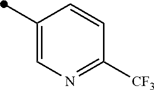 | Br |
| 170 | 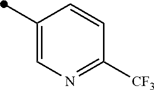 | OH |
| 171 | 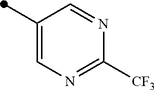 | F |
| 172 | 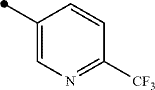 | OH |
| 173 | 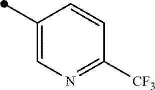 | NMe₂ |

TABLE 17

| Compound No. | |
|---|---|
| 174 | 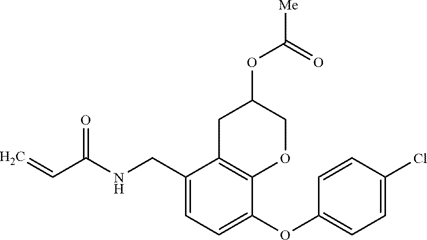 [SMILES: C=CC(=O)NCc1ccc(Oc2ccc(Cl)cc2)c2c1CC(OC(C)=O)CO2] |
| 175 | 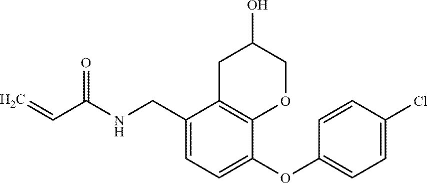 [SMILES: C=CC(=O)NCc1ccc(Oc2ccc(Cl)cc2)c2c1CC(O)CO2] |
| 176 | 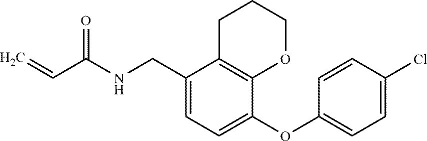 [SMILES: C=CC(=O)NCc1ccc(Oc2ccc(Cl)cc2)c2c1CCCO2] |
| 177 | 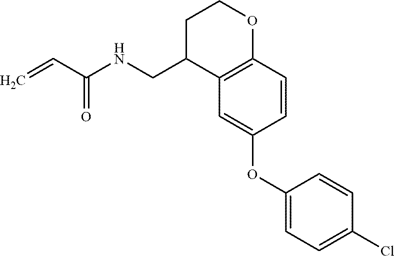 [SMILES: C=CC(=O)NCC1CCOc2ccc(Oc3ccc(Cl)cc3)cc21] |

TABLE18

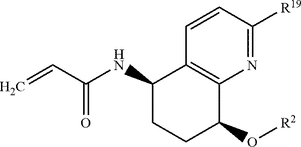

| Compound No. | R² | R¹⁰ |
|---|---|---|
| 178 | 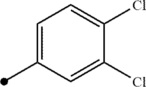 | Cl |
| 179 | 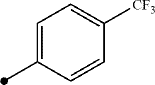 | OMe |

TABLE 18-continued

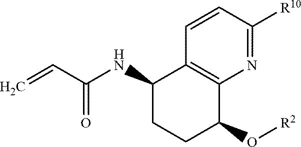

| Compound No. | R³ | R¹⁰ |
|---|---|---|
| 180 | 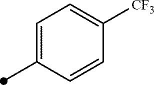 | 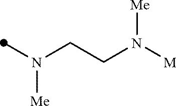 |
| 181 | 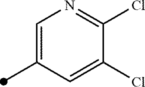 | Cl |
| 182 | 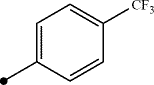 | CN |
| 183 | 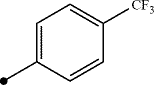 | Me |
| (Optionally gave substitute of 184) | | |
| 184 | 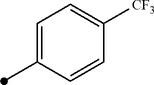 | Me |
| 185 | 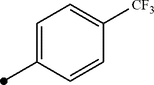 | 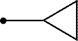 |
| 186 | 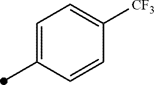 | Et |
| 187 | 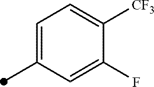 | Cl |
| 188 | 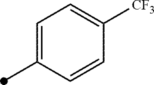 | OF₃ |
| 189 | 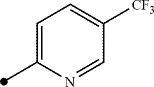 | Cl |

TABLE 19

| Compound No. | |
|---|---|
| 190 | 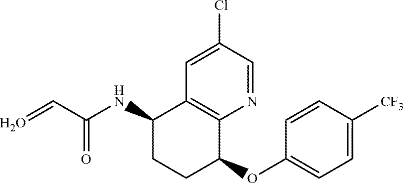 [SMILES: O=CC(=O)N[C@@H]1CC[C@H](Oc2ccc(C(F)(F)F)cc2)c2ncc(Cl)cc21] |
| 191 | 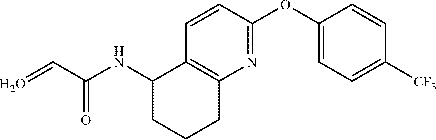 [SMILES: O=CC(=O)NC1CCCc2nc(Oc3ccc(C(F)(F)F)cc3)ccc21] |

TABLE 20

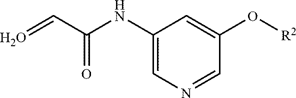

| Compound No. | R² |
|---|---|
| 192 | 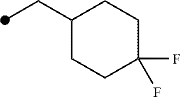 |
| 193 | 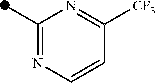 |
| 194 | 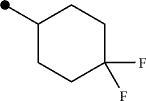 |

TABLE 21

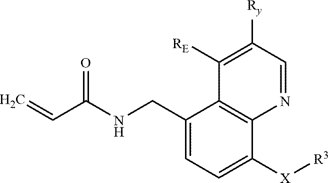

| Compound No. | R² | Rx | Ry | X |
|---|---|---|---|---|
| 195 | 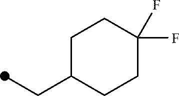 | H | H | 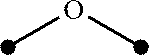 |

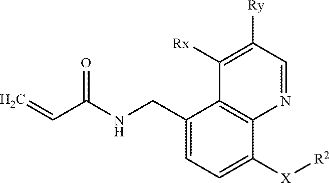

| Compound No. | R² | Rx | Ry | X |
|---|---|---|---|---|
| 196 | 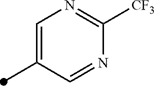 | H | H | 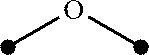 |
| 197 | 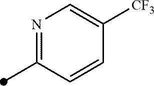 | H | H | 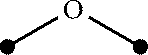 |
| 198 | 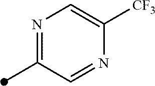 | H | H | 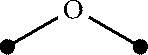 |
| 199 | 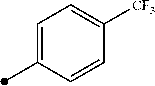 | H | Me | 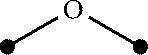 |
| 200 | 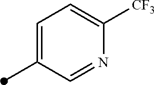 | H | Me | 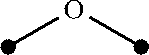 |
| 201 | 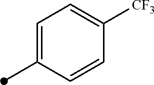 | Me | H | 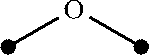 |
| 202 | 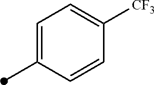 | H | H | 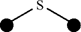 |
| 203 | 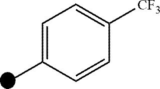 | H | H | 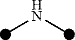 |
| 204 | 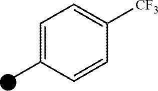 | H | H | 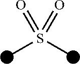 |

TABLE 22

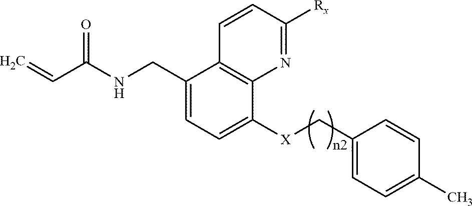

| Compound No. | R₂ | X | _{R}2 |
|---|---|---|---|
| 205 | H | 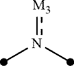 | 0 |
| 206 | H | 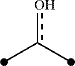 | 0 |
| 207 | H | 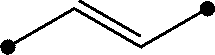 | 0 |
| 208 | H | 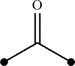 | 0 |
| 209 | H | 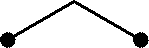 | |
| 210 | H | 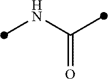 | 0 |
| 211 | H | 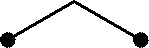 | 0 |
| 212 | Me | 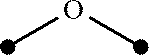 | 0 |
| 213 | OH | 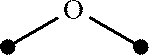 | 0 |

TABLE 23

| Com-Pound No. | |
|---|---|
| 214 | 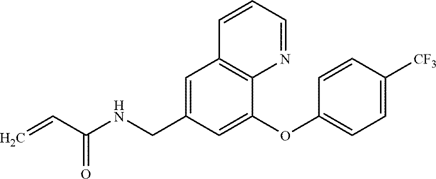 [SMILES: C=CC(=O)NCc1cc(Oc2ccc(C(F)(F)F)cc2)c2ncccc2c1] |
| 215 | 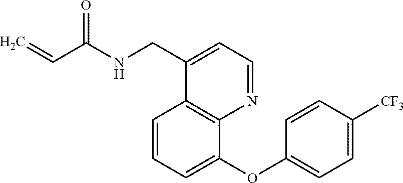 [SMILES: C=CC(=O)NCc1ccnc2c(Oc3ccc(C(F)(F)F)cc3)cccc12] |

| Com-pound No. | |
|---|---|
| 216 | 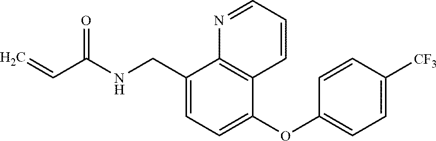 [SMILES: C=CC(=O)NCc1ccc(Oc2ccc(C(F)(F)F)cc2)c2cccnc12] |
| 217 | 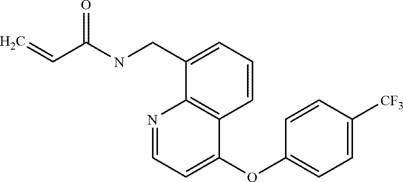 [SMILES: C=CC(=O)NCc1cccc2c(Oc3ccc(C(F)(F)F)cc3)ccnc12] |
| 218 | 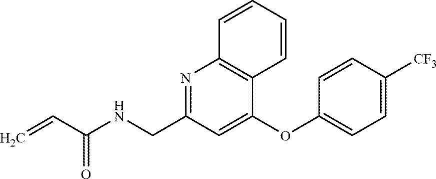 [SMILES: C=CC(=O)NCc1cc(Oc2ccc(C(F)(F)F)cc2)c2ccccc2n1] |
| 219 | 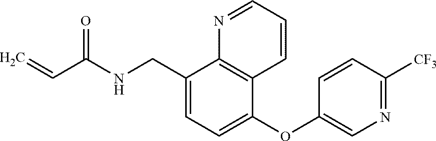 [SMILES: C=CC(=O)NCc1ccc(Oc2ccc(C(F)(F)F)nc2)c2cccnc12] |
| 220 | 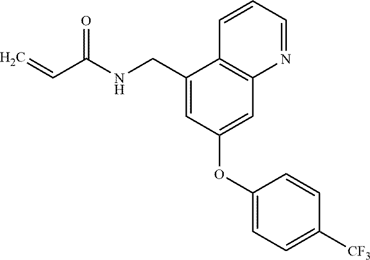 [SMILES: C=CC(=O)NCc1cc(Oc2ccc(C(F)(F)F)cc2)cc2ncccc12] |
| 221 | 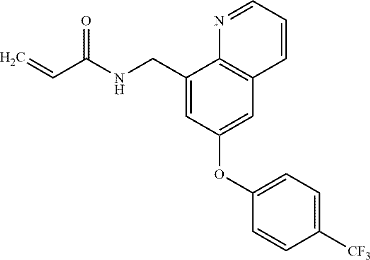 [SMILES: C=CC(=O)NCc1cc(Oc2ccc(C(F)(F)F)cc2)cc2cccnc12] |

TABLE 24

| Compound No. | |
|---|---|
| 222 | 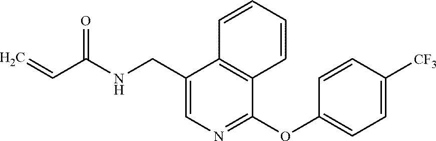 [SMILES: C=CC(=O)NCc1cnc(Oc2ccc(C(F)(F)F)cc2)c2ccccc12] |
| 223 | 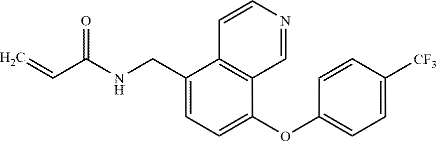 [SMILES: C=CC(=O)NCc1ccc(Oc2ccc(C(F)(F)F)cc2)c2cnccc12] |
| 224 | 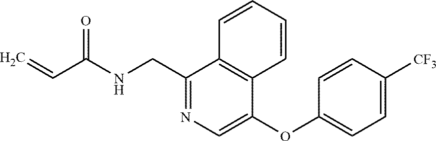 [SMILES: C=CC(=O)NCc1ncc(Oc2ccc(C(F)(F)F)cc2)c2ccccc12] |
| 225 | 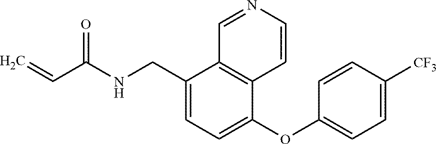 [SMILES: C=CC(=O)NCc1ccc(Oc2ccc(C(F)(F)F)cc2)c2ccncc12] |

TABLE 25

| Compound No. | |
|---|---|
| 226 | 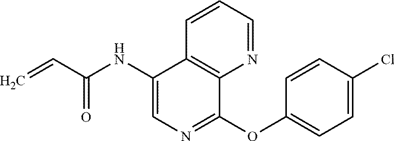 [SMILES: C=CC(=O)Nc1cnc(Oc2ccc(Cl)cc2)c2ncccc12] |
| 227 | 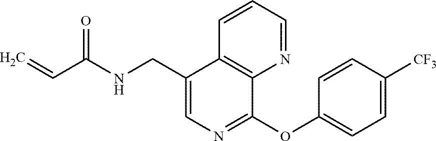 [SMILES: C=CC(=O)NCc1cnc(Oc2ccc(C(F)(F)F)cc2)c2ncccc12] |
| 228 | 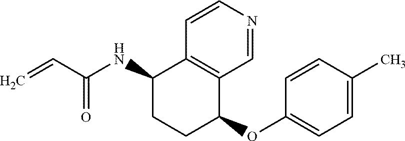 [SMILES: C=CC(=O)N[C@@H]1CC[C@H](Oc2ccc(C)cc2)c2cnccc21] |

TABLE 26

| Compound No. | |
|---|---|
| 229 | Compound with retention time of 2.61 minutes among two emntiomers contained in compound 51 |
| 230 | Compound with retention time of 3.28 minutes among two numbers combined in compound 51 |
| 231 | Compound with retention time of 2.44 minutes among two esmisuese combined in compound 153 |
| 232 | Compound with retention time of 1.24 minutes among two custiomers combined in compound 153 |
| 233 | Compound with retention time of 4.56 minutes among two containers combined in compound 40 |
| 234 | Compound with retention time of 3.07 minutes among two emntiomers combined in compound 40 |
| 235 | Compound with retention time of 3.57 minutes among two cusmtismere combined in compound 41 |
| 236 | Compound with retention time of 4.35 minutes Among two emmiomers combined in compound 41 |
| 237 | Compound with retention time of 3.14 minutes among two custiomers combined in compound 33 |
| 238 | Compound with retention time of 6.79 minutes among two esunisuence combined in compound 33 |
| 239 | Compound with retention time of 6.19 minutes among two costiomers combined in compound 31 |
| 240 | Compound with retention time of 2.43 minutes among two contiomers combined in compound 31 |
| 241 | Compound with retention time of 2.73 minutes among two emotiomers contained in compound 76 |
| 242 | Compound with retention time of 3.41 minutes among two custiomers combined in compound 76 |

); or PCT Application Publication No. WO 2018/204532 is not a compound of Tables 1-3, Examples 1-252 or A1 of WO 2018/204532; structures of which are shown above), published Nov. 8, 2018.

In certain embodiments, the compound of Formula (I-A), (I-B), or (II), is a compound provided in any one of the Examples below. In certain embodiments, the compound of Formula (I-A), (I-B), or (II) is a compound provided in Examples 1 and 2 below.

In certain embodiments, a compound described herein is a compound of Formula (I-A), (I-B), or (II), or a pharmaceutically acceptable salt, co-crystal, tautomer, stereoisomer, solvate, hydrate, polymorph, isotopically enriched derivative, or prodrug thereof. In certain embodiments, a compound described herein is a compound of Formula (I-A), (I-B), or (II), or a pharmaceutically acceptable salt thereof. In certain embodiments, a compound described herein is a compound of Formula (I-A), or a pharmaceutically acceptable salt thereof. In certain embodiments, a compound described herein is a compound of Formula (I-B), or a pharmaceutically acceptable salt thereof. In certain embodiments, a compound described herein is a compound of Formula (II), or a pharmaceutically acceptable salt thereof.

Certain compounds described herein bind, covalently modify, and/or inhibit a transcription factor. In certain embodiments, the compounds described herein irreversibly inhibit a transcription factor. In certain embodiments, the compounds described herein reversibly inhibit a transcription factor. In certain embodiments, the transcription factor is TEAD. In certain embodiments, the transcription factor is TEAD1. In certain embodiments, the transcription factor is TEAD2. In certain embodiments, the transcription factor is TEAD3. In certain embodiments, the transcription factor is TEAD4. In certain embodiments, the compounds described herein covalently bind to the transcription factor (e.g., TEAD, such as TEAD1, TEAD2, TEAD3, TEAD4). In certain embodiments, the compounds described herein reversibly bind to the transcription factor (e.g., TEAD, such as TEAD1, TEAD2, TEAD3, TEAD4). In certain embodiments, the compounds described herein non-reversibly bind to the transcription factor (e.g., TEAD, such as TEAD1, TEAD2, TEAD3, TEAD4). In certain embodiments, the compounds described herein modulate the activity of a transcription factor (e.g., TEAD, such as TEAD1, TEAD2, TEAD3, TEAD4). In certain embodiments, the compounds described herein inhibit a transcription factor (e.g., TEAD, such as TEAD1, TEAD2, TEAD3, TEAD4). In certain embodiments, the compounds described herein inhibit the activity of a transcription factor (e.g., TEAD, such as TEAD1, TEAD2, TEAD3, TEAD4). In certain embodiments, the compounds described herein reversibly inhibit the activity of a transcription factor (e.g., TEAD, such as TEAD1, TEAD2, TEAD3, TEAD4).

The binding affinity of a compound described herein to a transcription factor (e.g., TEAD, such as TEAD1, TEAD2, TEAD3, TEAD4) may be measured by the dissociation constant (K_{d}) value of an adduct of the compound and the TEAD using methods known in the art (e.g., isothermal titration calorimetry (ITC)). In certain embodiments, the K_{d} value of the adduct is not more than about 100 µM, not more than about 10 µM, not more than about 1 µM, not more than about 100 nM, not more than about 10 nM, or not more than about 1 nM.

In certain embodiments, the activity of a transcription factor (e.g., TEAD, such as TEAD1, TEAD2, TEAD3, TEAD4) is inhibited by a compound described herein. The inhibition of the activity of a transcription factor (e.g., TEAD, such as TEAD1, TEAD2, TEAD3, TEAD4) by a compound described herein may be measured by determining the half maximal inhibitory concentration (IC₅₀) of the compound when the compound, or a pharmaceutical composition thereof, is contacted with the transcription factor (e.g., TEAD, such as TEAD1, TEAD2, TEAD3, TEAD4). The IC₅₀ values may be obtained using methods known in the art (e.g., by a competition binding assay). In certain embodiments, the IC₅₀ value of a compound described herein is not more than about 1 mM, not more than about 100 µM, not more than about 10 µM, not more than about 1 µM, not more than about 100 nM, not more than about 10 nM, or not more than about 1 nM.

The compounds described herein may selectively modulate the activity of a transcription factor (e.g., TEAD, such as TEAD1, TEAD2, TEAD3, TEAD4). In certain embodiments, the compounds selectively inhibit a transcription factor (e.g., TEAD, such as TEAD1, TEAD2, TEAD3, TEAD4). In certain embodiments, the compounds selectively inhibit the activity of a transcription factor (e.g., TEAD, such as TEAD1, TEAD2, TEAD3, TEAD4). In certain embodiments, the compounds inhibit the activity of two or more transcription factors (e.g., TEAD, such as TEAD1, TEAD2, TEAD3, TEAD4) to the same extent.

The selectivity of a compound described herein in inhibiting the activity of a first transcription factor (e.g., TEAD, such as TEAD1, TEAD2, TEAD3, TEAD4) over a second transcription factor different from the first transcription factor (e.g., a different TEAD) may be measured by the quotient of the IC₅₀ value of the compound in inhibiting the activity of the second transcription factor different from the first transcription factor (e.g., a different TEAD) over the IC₅₀ value of the compound in inhibiting the activity of the first transcription factor (e.g., TEAD). The selectivity of a compound described herein in modulating the activity of a first transcription factor (e.g., TEAD) over a second transcription factor different from the first transcription factor (e.g., a different TEAD) may also be measured by the quotient of the K_{d} value of an adduct of the compound and the second transcription factor different from the first transcription factor (e.g., a different TEAD) over the K_{d} value of an adduct of the compound and the first transcription factor different from the first transcription factor (e.g., a different TEAD). In certain embodiments, the selectivity is at least about 1-fold, at least about 3-fold, at least about 10-fold, at least about 30-fold, at least about 100-fold, at least about 300-fold, at least about 1,000-fold, at least about 3,000-fold, at least about 10,000-fold, at least about 30,000-fold, or at least about 100,000-fold. In certain embodiments, the selectivity is at least about 2-fold, about 5-fold, about 10-fold, or more.

It is expected that the compounds described herein may be useful in treating and/or preventing diseases associated with aberrant activity (e.g., increased activity, undesired activity, abnormal activity) of a transcription factor (e.g., TEAD, such as TEAD1, TEAD2, TEAD3, TEAD4). It is known in the art that transcription factors are implicated in a wide range of diseases and conditions, such as proliferative diseases, inflammatory diseases, autoimmune diseases. Therefore, the compounds described herein are expected to be useful in treating and/or preventing diseases (e.g., proliferative diseases, inflammatory diseases, autoimmune diseases).

### Pharmaceutical Compositions, Kits, and Administration

The present disclosure also provides pharmaceutical compositions comprising a compound described herein and optionally a pharmaceutically acceptable excipient. In certain embodiments, a compound described herein is a compound of Formula (I-A), (I-B), or (II), or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, a therapeutically effective amount is an amount effective for inhibiting the aberrant activity of a transcription factor (e.g., TEAD, such as TEAD1, TEAD2, TEAD3, TEAD4). In certain embodiments, a therapeutically effective amount is an amount effective for treating a disease (e.g., a disease associated with aberrant activity of a transcription factor (e.g., TEAD (e.g., proliferative diseases, inflammatory diseases, autoimmune diseases)). In certain embodiments, a therapeutically effective amount is an amount effective for inhibiting the aberrant activity of a transcription factor (e.g., TEAD, such as TEAD1, TEAD2, TEAD3, TEAD4) and treating a disease (e.g., a disease associated with aberrant activity of a transcription factor (e.g., TEAD, such as TEAD1, TEAD2, TEAD3, TEAD4) (e.g., proliferative disease, inflammatory disease, autoimmune disease))). In certain embodiments, a therapeutically effective amount is an amount effective for inhibiting the transcription of a gene (e.g., a gene controlled or regulated by a transcription factor (e.g., TEAD, such as TEAD1, TEAD2, TEAD3, TEAD4)) in a subject and/or biological sample. In certain embodiments, a prophylactically effective amount is an amount effective for inhibiting the aberrant activity of a transcription factor (e.g., TEAD, such as TEAD1, TEAD2, TEAD3, TEAD4). In certain embodiments, a prophylactically effective amount is an amount effective for preventing or keeping a subject in need thereof in remission of a disease (e.g., a disease associated with aberrant activity of a transcription factor (e.g., TEAD) (e.g., proliferative disease, inflammatory disease, autoimmune disease)). In certain embodiments, a prophylactically effective amount is an amount effective for inhibiting the aberrant activity of a transcription factor (e.g., TEAD, such as TEAD1, TEAD2, TEAD3, TEAD4), and preventing or keeping a subject in need thereof in remission of a disease (e.g., a disease associated with aberrant activity of a transcription factor (e.g., TEAD, such as TEAD1, TEAD2, TEAD3, TEAD4) (e.g., proliferative disease, inflammatory disease, autoimmune disease)). In certain embodiments, a prophylactically effective amount is an amount effective for inhibiting the transcription of a gene (e.g., a gene controlled or regulated by a transcription factor (e.g., TEAD, such as TEAD1, TEAD2, TEAD3, TEAD4)) in a subject and/or biological sample.

In certain embodiments, the effective amount is an amount effective for inhibiting the activity of a transcription factor (e.g., TEAD, such as TEAD1, TEAD2, TEAD3, TEAD4) by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of a transcription factor (e.g., TEAD, such as TEAD1, TEAD2, TEAD3, TEAD4) by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for increasing the activity of a transcription factor (e.g., TEAD, such as TEAD1, TEAD2, TEAD3, TEAD4) by at least 10%, at least 20%, at least 30%, at least 40%, at least 50%, at least 60%, at least 70%, at least 80%, at least 90%, at least 95%, or at least 98%. In certain embodiments, the effective amount is an amount effective for increasing the activity of a transcription factor (e.g., TEAD, such as TEAD1, TEAD2, TEAD3, TEAD4) by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell being contacted with a compound or pharmaceutical composition thereof described herein is in vitro. In certain embodiments, the cell being contacted with a compound or pharmaceutical composition thereof described herein is in vivo.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween^{®} 20), polyoxyethylene sorbitan (Tween^{®} 60), polyoxyethylene sorbitan monooleate (Tween^{®} 80), sorbitan monopalmitate (Span^{®} 40), sorbitan monostearate (Span^{®} 60), sorbitan tristearate (Span^{®} 65), glyceryl monooleate, sorbitan monooleate (Span^{®} 80), polyoxyethylene esters (e.g., polyoxyethylene monostearate (Myrj^{®} 45), polyoxyethylene hydrogenated castor oil, polyethoxylated castor oil, polyoxymethylene stearate, and Solutol^{®}), sucrose fatty acid esters, polyethylene glycol fatty acid esters (e.g., Cremophor^{®}), polyoxyethylene ethers, (e.g., polyoxyethylene lauryl ether (Brij^{®} 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic^{®} F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum^{®}), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant^{®} Plus, Phenonip^{®}, methylparaben, Germall^{®} 115, Germaben^{®} II, Neolone^{®}, Kathon^{®}, and Euxyl^{®}.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer’s solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor^{®}, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer’s solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample (e.g., tissue, cell), any two doses of the multiple doses include different or substantially the same amounts of a compound described herein. In certain embodiments, when multiple doses are administered to a subject or applied to a biological sample (e.g., tissue, cell), the frequency of administering the multiple doses to the subject or applying the multiple doses to the biological sample (e.g., tissue, cell) is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample (e.g., tissue, cell) is one dose per day. In certain embodiments, the frequency of administering the multiple doses to the subject or applying the multiple doses to the biological sample (e.g., tissue, cell) is two doses per day. In certain embodiments, the frequency of administering the multiple doses to the subject or applying the multiple doses to the biological sample (e.g., tissue, cell) is three doses per day. In certain embodiments, when multiple doses are administered to a subject or applied to a biological sample (e.g., tissue, cell), the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 µg and 1 µg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or pharmaceutical composition thereof, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inhibiting the activity of a transcription factor (e.g., TEAD, such as TEAD1, TEAD2, TEAD3, TEAD4)) in a subject and/or biological sample (e.g., tissue, cell)), improve bioavailability, improve safety, reduce drug resistance, reduce and/or modify metabolism, inhibit excretion, and/or modify distribution in a subject and/or biological sample (e.g., tissue, cell). It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compounds described herein and the additional pharmaceutical agent, but not both.

The compound or pharmaceutical composition thereof can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, inflammatory disease, autoimmune disease). Each additional pharmaceutical agent may be administered at a dose and/or on a time schedule determined for that pharmaceutical agent. The additional pharmaceutical agents may also be administered together with each other and/or with the compound or pharmaceutical composition thereof described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia Chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIBO (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In certain embodiments, the additional pharmaceutical agent is an anti-lymphoma agent. In certain embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmustine), BELEODAQ (belinostat), BEXXAR (tositumomab and iodine I131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R-CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In certain embodiments, the additional pharmaceutical agent is REVLIMID (lenalidomide), DACOGEN (decitabine), VIDAZA (azacitidine ), CYTOSAR-U (cytarabine), IDAMYCIN (idarubicin ), CERUBIDINE (daunorubicin), LEUKERAN (chlorambucil), NEOSAR (cyclophosphamide), FLUDARA (fludarabine), LEUSTATIN (cladribine), or a combination thereof. In certain embodiments, the additional pharmaceutical agent is ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI , FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is ibrutinib. In certain embodiments, the additional pharmaceutical agent is a transcription factor inhibitor (e.g., inhibitor of EGFR and/or MEK). In certain embodiments, the additional pharmaceutical agent is an inhibitor of a gene and/or protein in the Hippo signaling pathway. In certain embodiments, the additional pharmaceutical agent is an inhibitor of EGFR (e.g., osimertinib, gefitinib) and/or an inhibitor of MEK (e.g., trametinib, selumetinib). In certain embodiments, the additional pharmaceutical agent is an inhibitor of EGFR (e.g., osimertinib, gefitinib). In certain embodiments, the additional pharmaceutical agent comprises an inhibitor of MEK (e.g., trametinib, selumetinib). In certain embodiments, the additional pharmaceutical agent comprises an inhibitor of tankyrase inhibitor and/or an indirect inhibitor of YAP (e.g., compound XAV939). In certain embodiments, the additional pharmaceutical agent is an anti-proliferative agent (e.g., anti-cancer agent, such as an inhibitor of EGFR, an inhibitor of MEK, or an inhibitor of EGFR and an inhibitor of MEK). In certain embodiments, the additional pharmaceutical agent is a transcription factor inhibitor (e.g., inhibitor of EGFR and/or MEK). In certain embodiments, the additional pharmaceutical agent is an agent for treating lung cancer (e.g., non-small cell lung cancer (NSCLC)). In certain embodiments, the additional pharmaceutical agent is an agent for treating lung cancer (e.g., non-small cell lung cancer (NSCLC)), such as NSCLC with a mutation in a gene and/or protein in the Hippo signaling pathway (e.g., mutation in EGFR). In certain embodiments, the additional pharmaceutical agent is a kinase inhibitor. In certain embodiments, the additional pharmaceutical agent is a tyrosine kinase inhibitor (TKI). In certain embodiments, the additional pharmaceutical agent is an agent for treating a cancer that has a mutation in a gene of the Hippo signaling pathway. In certain embodiments, the additional pharmaceutical agent is an agent for treating a cancer that has a mutation in EGFR. In certain embodiments, the additional pharmaceutical agent is an agent for treating a cancer that has a mutation in MEK. In certain embodiments, the additional pharmaceutical agent is an agent for treating a cancer that is an EGFR-mutant non-small cell lung cancer. In certain embodiments, the additional pharmaceutical agent is an agent for treating a cancer that is resistant to certain anti-proliferative agents (e.g., cancers resistant to inhibitors of EGFR, such as osimertinib, and/or inhibitors of MEK, such as trametinib). In certain embodiments, the additional pharmaceutical agent is an agent for treating a cancer that is resistant to inhibitors of EGFR and/or MEK. In certain embodiments, the additional pharmaceutical agent is an agent for treating a cancer that is resistant to osimertinib and trametinib. In certain embodiments, the additional pharmaceutical agent is an agent for treating a cancer that is resistant to tyrosine kinase inhibitors (TKI’s).

In certain embodiments, the additional pharmaceutical agent is a binder or inhibitor of TEAD (e.g., TEAD1, TEAD2, TEAD3, TEAD4)). In certain embodiments, the additional pharmaceutical agent is a binder or inhibitor of TEAD. In certain embodiments, the additional pharmaceutical agent is a binder or inhibitor of TEAD1. In certain embodiments, the additional pharmaceutical agent is a binder or inhibitor of TEAD2. In certain embodiments, the additional pharmaceutical agent is a binder or inhibitor of TEAD3. In certain embodiments, the additional pharmaceutical agent is a binder or inhibitor of TEAD4. In certain embodiments, the additional pharmaceutical agent is a binder or inhibitor of Bruton’s tyrosine kinase (BTK). In certain embodiments, the additional pharmaceutical agent is selected from the group consisting of epigenetic and transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, inflammatory disease, autoimmune disease) in a subject in need thereof. In certain embodiments, the kits are useful for preventing a disease (e.g., proliferative disease, inflammatory disease, autoimmune disease) in a subject in need thereof. In certain embodiments, the kits are useful for inhibiting the activity (e.g., aberrant or unwanted activity, such as increased activity) of a transcription factor (e.g., TEAD, such as TEAD1, TEAD2, TEAD3, TEAD4) in a subject and/or biological sample (e.g., tissue, cell). In certain embodiments, the kits are useful for inhibiting the transcription of a gene (e.g., a gene controlled or regulated by a transcription factor (e.g., TEAD, such as TEAD1, TEAD2, TEAD3, TEAD4)) in a subject and/or biological sample.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, inflammatory disease, autoimmune disease) in a subject in need thereof. In certain embodiments, the kits and instructions provide for preventing a disease (e.g., proliferative disease, inflammatory disease, autoimmune disease) in a subject in need thereof. In certain embodiments, the kits and instructions provide for modulating (e.g., inhibiting) the activity (e.g., aberrant activity, such as increased activity) of a transcription factor (e.g., TEAD, such as TEAD1, TEAD2, TEAD3, TEAD4) in a subject and/or biological sample (e.g., tissue, cell). In certain embodiments, the kits and instructions provide for inhibiting the transcription of a gene (e.g., a gene controlled or regulated by a transcription factor (e.g., TEAD, such as TEAD1, TEAD2, TEAD3, TEAD4)) in a subject and/or biological sample. A kit described herein may include one or more additional pharmaceutical agents described herein as a separate composition.

### Methods of Treatment and Uses

The present disclosure provides methods of modulating (e.g., inhibiting or increasing) the activity (e.g., aberrant activity, such as increased or decreased activity) of a transcription factor (e.g., TEAD, such as TEAD1, TEAD2, TEAD3, TEAD4) using compounds described herein, which may be optionally administered in combination with an additional pharmaceutical agent, for example, modulators of other transcription factors (e.g., YAP, EGFR, MEK). The present disclosure provides methods of modulating (e.g., inhibiting or increasing) the activity (e.g., aberrant activity, such as increased or decreased activity) of a transcription factor (e.g., TEAD, such as TEAD1, TEAD2, TEAD3, TEAD4) in a subject and/or biological sample (e.g., tissue, cell), using compounds described herein, which may be optionally administered in combination with an additional pharmaceutical agent, for example, modulators of other transcription factors (e.g., YAP, EGFR, MEK). The present disclosure also provides methods for the treatment of a wide range of diseases, such as diseases associated with the aberrant activity (e.g., increased activity) of a transcription factor (e.g., TEAD, such as TEAD1, TEAD2, TEAD3, TEAD4), using compounds described herein, which may be optionally administered in combination with an additional pharmaceutical agent, for example, modulators of other transcription factors (e.g., YAP, EGFR, MEK), for example, for treating proliferative diseases, inflammatory diseases, and/or autoimmune diseases in a subject in need thereof. The present disclosure provides methods for the treatment and/or prevention of a proliferative disease (e.g., cancers (e.g., carcinoma, sarcoma); lung cancer, breast cancer, liver cancer, pancreatic cancer, gastric cancer, ovarian cancer, colon cancer, colorectal cancer, skin cancer, esophageal cancer)), inflammatory disease (e.g., fibrosis), or autoimmune disease (e.g., sclerosis), using compounds described herein, which may be optionally administered in combination with an additional pharmaceutical agent, for example, modulators of other transcription factors (e.g., YAP, EGFR, MEK). The present disclosure provides methods for inhibiting the transcription of a gene (e.g., a gene controlled or regulated by a transcription factor (e.g., TEAD, such as TEAD1, TEAD2, TEAD3, TEAD4)) in a subject and/or biological sample (e.g., tissue, cell), using compounds described herein, which may be optionally administered in combination with an additional pharmaceutical agent, for example, modulators of other transcription factors (e.g., YAP, EGFR, MEK).

The present disclosure also provides a compound of Formula (I-A), (I-B), or (II), or a pharmaceutically acceptable salt, solvate, hydrate, polymorph, co-crystal, tautomer, stereoisomer, isotopically labeled derivative, prodrug, or pharmaceutical composition thereof, which may be optionally administered in combination with an additional pharmaceutical agent, for example, modulators of other transcription factors (e.g., YAP, EGFR, MEK), for use in the treatment of diseases, such as proliferative diseases, inflammatory diseases, autoimmune diseases, in a subject in need thereof.

The present disclosure also provides uses of a compound of Formula (I-A), (I-B), or (II), or a pharmaceutically acceptable salt, solvate, hydrate, polymorph, co-crystal, tautomer, stereoisomer, isotopically labeled derivative, prodrug, or pharmaceutical composition thereof, which may be optionally administered in combination with an additional pharmaceutical agent, for example, modulators of other transcription factors (e.g., YAP, EGFR, MEK), in the manufacture of a medicament for the treatment of various diseases, such as proliferative diseases, inflammatory diseases, and autoimmune diseases, in a subject in need thereof.

In another aspect, the present disclosure provides methods of modulating the activity of a transcription factor (e.g., TEAD, such as TEAD1, TEAD2, TEAD3, TEAD4) in a subject and/or biological sample (e.g., cell, tissue) using compounds described herein, which may be optionally administered in combination with an additional pharmaceutical agent, for example, modulators of other transcription factors (e.g., YAP, EGFR, MEK). In certain embodiments, provided are methods of inhibiting the activity of a transcription factor (e.g., TEAD) in a subject. In certain embodiments, provided are methods of inhibiting the activity of a transcription factor (e.g., TEAD) in a cell. In certain embodiments, provided are methods of increasing the activity of a transcription factor (e.g., TEAD, such as TEAD1, TEAD2, TEAD3, TEAD4) in a subject. The compounds described herein may exhibit transcription factor inhibitory activity; the ability to inhibit TEAD; the ability to inhibit TEAD1, without inhibiting another transcription factor (e.g., a different TEAD); the ability to inhibit TEAD2, without inhibiting another transcription factor (e.g., a different TEAD); the ability to inhibit TEAD3, without inhibiting another transcription factor (e.g., a different TEAD); the ability to inhibit TEAD4, without inhibiting another transcription factor (e.g., a different TEAD); a therapeutic effect and/or preventative effect in the treatment of cancers; a therapeutic effect and/or preventative effect in the treatment of proliferative diseases, inflammatory diseases, and/or autoimmune diseases; and/or a therapeutic profile (e.g., optimum safety and curative effect) that is superior to existing chemotherapeutic agents, or agents for treating inflammatory diseases and/or autoimmune diseases.

In certain embodiments, provided are methods of decreasing the activity of a transcription factor (e.g., TEAD, such as TEAD1, TEAD2, TEAD3, TEAD4) in a subject or biological sample (e.g., cell, tissue) by a method described herein by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a transcription factor (e.g., TEAD, such as TEAD1, TEAD2, TEAD3, TEAD4) in a subject or biological sample (e.g., cell, tissue)is decreased by a method described herein by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a transcription factor (e.g., TEAD, such as TEAD1, TEAD2, TEAD3, TEAD4) in a subject or biological sample (e.g., cell, tissue) is selectively inhibited by the compound. In some embodiments, the activity of a transcription factor (e.g., TEAD, such as TEAD1, TEAD2, TEAD3, TEAD4) in a subject or biological sample (e.g., cell, tissue) is selectively decreased by the compound.

Without wishing to be bound by any particular theory, the compounds described herein are able to bind (e.g., covalently modify) the transcription factor being inhibited. In certain embodiments, a compound described herein is able to bind (e.g., covalently modify) the transcription factor. In certain embodiments, the compound described herein is able to covalently bind a cysteine residue of the transcription factor. In certain embodiments, the compound described herein is able to covalently bind a cysteine residue of TEAD. In certain embodiments, the compound described herein is able to covalently bind a cysteine residue of TEAD1. In certain embodiments, the compound is capable of covalently binding cysteine 359 of TEAD1. In certain embodiments, the compound described herein is able to covalently bind a cysteine residue of TEAD2. In certain embodiments, the compound is capable of covalently binding cysteine 380 of TEAD2. In certain embodiments, the compound is capable of covalently binding TEAD1. In certain embodiments, the compound is capable of covalently binding TEAD2. In certain embodiments, the compound is capable of covalently binding TEAD3. In certain embodiments, the compound described herein is able to covalently bind a cysteine residue of TEAD4. In certain embodiments, the compound is capable of covalently binding TEAD4. In certain embodiments, the compound is capable of binding the YAP/TAZ domain of a TEAD family transcription factor. In certain embodiments, the compound is capable of covalently modifying TEAD1 (e.g., C359 of TEAD1). In certain embodiments, the compound is capable of covalently modifying TEAD2 (e.g., C380 of TEAD2). In certain embodiments, the compound is capable of covalently modifying C359 (cysteine 359) of TEAD1. In certain embodiments, the compound is capable of covalently modifying C380 (cysteine 380) of TEAD2. In certain embodiments, the compound is capable of covalently modifying TEAD3. In certain embodiments, the compound is capable of covalently modifying TEAD4. In certain embodiments, the compound is capable of covalently modifying TEAD1. In certain embodiments, the compound is capable of covalently modifying TEAD2. In certain embodiments, the compound is capable of covalently modifying TEAD3. In certain embodiments, the compound is capable of covalently modifying TEAD4. In certain embodiments, the compound is capable of non-covalently inhibiting TEAD1. In certain embodiments, the compound is capable of non-covalently inhibiting TEAD2. In certain embodiments, the compound is capable of non-covalently inhibiting TEAD3. In certain embodiments, the compound is capable of non-covalently inhibiting TEAD4.

In another aspect, the present disclosure provides methods of inhibiting a transcription factor (e.g., TEAD, such as TEAD1, TEAD2, TEAD3, TEAD4) in a subject, the methods comprising administering to the subject an effective amount (e.g., therapeutically effective amount) of a compound, or pharmaceutical composition thereof, as described herein. In another aspect, the present disclosure provides methods of inhibiting the activity of a transcription factor (e.g., TEAD, such asTEAD1, TEAD2, TEAD3, TEAD4) in a subject, the methods comprising administering to the subject an effective amount (e.g., therapeutically effective amount) of a compound, or pharmaceutical composition thereof, as described herein. In another aspect, the present disclosure provides methods of inhibiting the activity of a transcription factor (e.g., TEAD, such as TEAD1, TEAD2, TEAD3, TEAD4) in a biological sample, the methods comprising contacting the biological sample with an effective amount of a compound, or pharmaceutical composition thereof, as described herein. In another aspect, the present disclosure provides methods of inhibiting the activity of a transcription factor (e.g., TEAD, such as TEAD1, TEAD2, TEAD3, TEAD4) in a biological sample (e.g., tissue, cell), the methods comprising contacting the biological sample (e.g., tissue, cell) with an effective amount of a compound, or pharmaceutical composition thereof, as described herein.

In another aspect, the present disclosure provides methods of inhibiting the activity of a transcription factor (e.g., TEAD, such as TEAD1, TEAD2, TEAD3, TEAD4) in a cell, the methods comprising contacting the cell with an effective amount of a compound, or pharmaceutical composition thereof, as described herein.

In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a subject, the methods comprising administering to the subject an effective amount (e.g., therapeutically effective amount) of a compound, or pharmaceutical composition thereof, as described herein. In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a subject, the methods comprising administering to the subject an effective amount (e.g., therapeutically effective amount) of a compound, or pharmaceutical composition thereof, as described herein. In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a biological sample, the methods comprising contacting the biological sample with an effective amount of a compound, or pharmaceutical composition thereof, as described herein. In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a biological sample (e.g., tissue, cell), the methods comprising contacting the biological sample (e.g., tissue, cell) with an effective amount of a compound, or pharmaceutical composition thereof, as described herein. In another aspect, the present disclosure provides methods of inhibiting the transcription of a gene (e.g., a gene controlled or regulated by a transcription factor (e.g., TEAD, such as TEAD1, TEAD2, TEAD3, TEAD4)) in a cell, the methods comprising contacting the cell with an effective amount of a compound, or pharmaceutical composition thereof, as described herein.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal, such as a transgenic mouse or transgenic pig.

In certain embodiments, the biological sample being contacted with the compound or pharmaceutical composition thereof is breast tissue, bone marrow, lymph node, lymph tissue, spleen, or blood. In certain embodiments, the biological sample being contacted with the compound or pharmaceutical composition thereof is a tumor or cancerous tissue. In certain embodiments, the biological sample being contacted with the compound or pharmaceutical composition thereof is serum, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from thebiological sample.

In certain embodiments, the cell or tissue being contacted with the compound or pharmaceutical composition thereof is present in vitro. In certain embodiments, the cell or tissue being contacted with the compound or pharmaceutical composition thereof is present in vivo. In certain embodiments, the cell or tissue being contacted with the compound or pharmaceutical composition thereof is present ex vivo. In certain embodiments, the cell or tissue being contacted with the compound or pharmaceutical composition thereof is a malignant cell (e.g., malignant blood cell). In certain embodiments, the cell being contacted with the compound or pharmaceutical composition thereof is a malignant hematopoietic stem cell (e.g., malignant myeloid cell or malignant lymphoid cell). In certain embodiments, the cell being contacted with the compound or pharmaceutical composition thereof is a malignant lymphocyte (e.g., malignant T-cell or malignant B-cell). In certain embodiments, the cell being contacted with the compound or pharmaceutical composition thereof is a malignant white blood cell. In certain embodiments, the cell being contacted with the compound or pharmaceutical composition thereof is a malignant neutrophil, malignant macrophage, or malignant plasma cell. In certain embodiments, the cell being contacted with the compound or pharmaceutical composition thereof is a carcinoma cell. In certain embodiments, the cell being contacted with the compound or pharmaceutical composition thereof is a breast carcinoma cell. In certain embodiments, the cell being contacted with the compound or pharmaceutical composition thereof is a sarcoma cell. In certain embodiments, the cell being contacted with the compound or pharmaceutical composition thereof is a sarcoma cell from breast tissue. In certain embodiments, the biological sample is from tissue or cells with cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma). In certain embodiments, the biological sample is from tissue or cells with an inflammatory disease or autoimmune disease. In certain embodiments, the biological sample is from tissue or cells with cancer (e.g., sarcoma, lung cancer, thyroid cancer, breast cancer, liver cancer, pancreatic cancer, gastric cancer, ovarian cancer, colon cancer, colorectal cancer, skin cancer, esophageal cancer; carcinoma), an inflammatory disease, or an autoimmune disease.

The disease (e.g., proliferative disease, inflammatory disease, autoimmune disease) to be treated or prevented using the compounds described herein may be associated with increased activity of a transcription factor, such as TEAD (e.g., TEAD1, TEAD2, TEAD3, TEAD4). The disease (e.g., proliferative disease, inflammatory disease, autoimmune disease) to be treated or prevented using the compounds described herein may be associated with the overexpression of a transcription factor, such as TEAD (e.g., TEAD1, TEAD2, TEAD3, TEAD4).

In certain embodiments, the disease (e.g., proliferative disease, inflammatory disease, autoimmune disease) to be treated or prevented using the compounds described herein may be associated with the overexpression of a transcription factor (e.g., TEAD, such as TEAD1, TEAD2, TEAD3, TEAD4). A disease (e.g., proliferative disease, inflammatory disease, autoimmune disease) may be associated with aberrant activity of a transcription factor (e.g., TEAD, such as TEAD1, TEAD2, TEAD3, TEAD4). Aberrant activity of a transcription factor (e.g., TEAD, such as TEAD1, TEAD2, TEAD3, TEAD4) may be elevated and/or inappropriate or undesired activity of the transcription factor (e.g., TEAD). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a transcription factor (e.g., TEAD, such as TEAD1, TEAD2, TEAD3, TEAD4) and be useful in treating and/or preventing diseases (e.g., proliferative diseases, inflammatory diseases, autoimmune diseases). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a transcription factor (e.g., TEAD) and be useful in treating and/or preventing diseases (e.g., proliferative disease, inflammatory disease, autoimmune disease). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a transcription factor (e.g., TEAD) and be useful in treating and/or preventing a disease (e.g., proliferative disease, inflammatory disease, autoimmune disease).

All types of biological samples described herein or known in the art are contemplated as being within the scope of the invention. In certain embodiments, the disease (e.g., proliferative disease, inflammatory disease, autoimmune disease) to be treated or prevented using the compounds described herein is cancer. All types of cancers disclosed herein or known in the art are contemplated as being within the scope of the invention. In certain embodiments, the proliferative disease is a hematological malignancy. In certain embodiments, the proliferative disease is a blood cancer. In certain embodiments, the proliferative disease is a hematological malignancy. In certain embodiments, the proliferative disease is leukemia. In certain embodiments, the proliferative disease is chronic lymphocytic leukemia (CLL). In certain embodiments, the proliferative disease is acute lymphoblastic leukemia (ALL). In certain embodiments, the proliferative disease is T-cell acute lymphoblastic leukemia (T-ALL). In certain embodiments, the proliferative disease is chronic myelogenous leukemia (CML). In certain embodiments, the proliferative disease is acute myeloid leukemia (AML). In certain embodiments, the proliferative disease is acute monocytic leukemia (AMoL). In certain embodiments, the proliferative disease is Waldenström’s macroglobulinemia. In certain embodiments, the proliferative disease is Waldenström’s macroglobulinemia associated with the MYD88 L265P somatic mutation. In certain embodiments, the proliferative disease is myelodysplastic syndrome (MDS). In certain embodiments, the proliferative disease is a carcinoma. In certain embodiments, the proliferative disease is lymphoma. In certain embodiments, the proliferative disease is T-cell lymphoma. In some embodiments, the proliferative disease is Burkitt’s lymphoma. In certain embodiments, the proliferative disease is a Hodgkin’s lymphoma. In certain embodiments, the proliferative disease is a non-Hodgkin’s lymphoma. In certain embodiments, the proliferative disease is multiple myeloma. In certain embodiments, the proliferative disease is melanoma. In certain embodiments, the proliferative disease is colorectal cancer. In certain embodiments, the proliferative disease is colon cancer. In certain embodiments, the proliferative disease is breast cancer. In certain embodiments, the proliferative disease is recurring breast cancer. In certain embodiments, the proliferative disease is mutant breast cancer. In certain embodiments, the proliferative disease is HER2+ breast cancer. In certain embodiments, the proliferative disease is HER2- breast cancer. In certain embodiments, the proliferative disease is triple-negative breast cancer (TNBC). In certain embodiments, the proliferative disease is a bone cancer. In certain embodiments, the proliferative disease is osteosarcoma. In certain embodiments, the proliferative disease is Ewing’s sarcoma. In some embodiments, the proliferative disease is a brain cancer. In some embodiments, the proliferative disease is neuroblastoma. In some embodiments, the proliferative disease is a lung cancer. In some embodiments, the proliferative disease is small cell lung cancer (SCLC). In some embodiments, the proliferative disease is non-small cell lung cancer (NSCLC). In certain embodiments, the lung cancer is mesothelioma. In certain embodiments, the cancer is a thyroid cancer. In certain embodiments, the cancer is a sarcoma. In certain embodiments, the sarcoma is Kaposi’s sarcoma. In certain embodiments, the cancer is fallopian tube cancer. In certain embodiments, the cancer is a carcinoma. In certain embodiments, the carcinoma is fallopian tube carcinoma. In some embodiments, the proliferative disease is liver cancer. In some embodiments, the proliferative disease is prostate cancer. In some embodiments, the proliferative disease is pancreatic cancer. In some embodiments, the proliferative disease is gastric cancer. In some embodiments, the proliferative disease is ovarian cancer. In some embodiments, the proliferative disease is ovarian cancer. In some embodiments, the cancer is skin cancer. In some embodiments, the cancer is esophageal cancer. In certain embodiments, the cancer has a mutation in a gene of the Hippo signaling pathway. In certain embodiments, the cancer has a mutation in EGFR. In certain embodiments, the cancer has a mutation in MEK. In certain embodiments, the cancer is an EGFR-mutant non-small cell lung cancer. In certain embodiments, the cancer is resistant to certain anti-proliferative agents (e.g., cancers resistant to inhibitors of EGFR and/or MEK). In certain embodiments, the cancer is resistant to inhibitors of EGFR and/or MEK. In certain embodiments, the cancer is resistant to tyrosine kinase inhibitors (TKI’s). In some embodiments, the proliferative disease is a benign neoplasm. All types of benign neoplasms disclosed herein or known in the art are contemplated as being within the scope of the invention. In some embodiments, the proliferative disease is associated with angiogenesis. All types of angiogenesis disclosed herein or known in the art are contemplated as being within the scope of the invention. In certain embodiments, the cancer is a sarcoma, lung cancer, thyroid cancer, breast cancer, liver cancer, pancreatic cancer, gastric cancer, ovarian cancer, colon cancer, colorectal cancer, skin cancer, esophageal cancer; a carcinoma; has a mutation in a gene of the Hippo signaling pathway (e.g., has a mutation in EGFR, such as an EGFR-mutant non-small cell lung cancer, or has a mutation in MEK), is a cancer resistant to certain anti-proliferative agents (e.g., cancers resistant to inhibitors of EGFR and/or MEK), or is a cancer is resistant to tyrosine kinase inhibitors (TKI’s). In certain embodiments, the cancer to be treated with a compound described herein along with an additional pharmaceutical agent, for example, a modulator of another transcription factors (e.g., YAP, EGFR, MEK), is a cancer that has a mutation in a gene of the Hippo signaling pathway (e.g., has a mutation in EGFR, such as an EGFR-mutant non-small cell lung cancer, or has a mutation in MEK). In certain embodiments, the cancer to be treated with a compound described herein along with an additional pharmaceutical agent, for example, a modulator of another transcription factors (e.g., YAP, EGFR, MEK), is a cancer resistant to certain anti-proliferative agents (e.g., cancers resistant to inhibitors of EGFR and/or MEK). In certain embodiments, the cancer to be treated with a compound described herein along with an additional pharmaceutical agent, for example, a modulator of another transcription factors (e.g., YAP, EGFR, MEK), is a cancer is resistant to tyrosine kinase inhibitors (TKI’s).

In certain embodiments, the inflammatory disease to be treated or prevented using the compounds described herein is fibrosis (e.g., idiopathic pulmonary fibrosis, liver cirrhosis, cystic fibrosis, systemic sclerosis, progressive kidney disease, or cardiovascular fibrosis). In certain embodiments, the autoimmune disease to be treated or prevented using the compounds described herein is sclerosis (e.g., systemic sclerosis (scleroderma) or multiple sclerosis). In certain embodiments, the autoimmune disease is amyotrophic lateral sclerosis.

Another aspect of the disclosure relates to methods of inhibiting the activity of a transcription factor (e.g., TEAD, such as TEAD1, TEAD2, TEAD3, TEAD4) in a biological sample (e.g., tissue, cell), or subject. In certain embodiments, the transcription factor is a TEAD. In certain embodiments, the TEAD is TEAD1. In certain embodiments, the TEAD is TEAD2. In certain embodiments, the TEAD is TEAD3. In certain embodiments, the TEAD is TEAD4. In certain embodiments, the activity of the transcription factor (e.g., TEAD, such as TEAD1, TEAD2, TEAD3, TEAD4) is aberrant activity of the transcription factor (e.g., TEAD, such as TEAD1, TEAD2, TEAD3, TEAD4). In certain embodiments, the activity of the transcription factor is increased activity of the transcription factor (e.g., TEAD). In certain embodiments, the inhibition of the activity of the transcription factor (e.g., TEAD, such as TEAD1, TEAD2, TEAD3, TEAD4) is irreversible. In other embodiments, the inhibition of the activity of the transcription factor (e.g., TEAD, such as TEAD1, TEAD2, TEAD3, TEAD4) is reversible. In certain embodiments, the methods of inhibiting the activity of the transcription factor (e.g., TEAD, such as TEAD1, TEAD2, TEAD3, TEAD4) include attaching a compound described herein to the transcription factor (e.g., TEAD, such as TEAD1, TEAD2, TEAD3, TEAD4). In certain embodiments, the methods comprise covalently modifying a transcription factor (e.g., TEAD, such as TEAD1, TEAD2, TEAD3, TEAD4) by attaching a compound described herein to the transcription factor (e.g., TEAD, such as TEAD1, TEAD2, TEAD3, TEAD4). In certain embodiments, the methods comprise covalently inhibiting a transcription factor (e.g., TEAD, such as TEAD1, TEAD2, TEAD3, TEAD4). In certain embodiments, the methods comprise reversibly inhibiting a transcription factor (e.g., TEAD, such as TEAD1, TEAD2, TEAD3, TEAD4). The present invention provides methods of inhibiting cell growth in a biological sample (e.g., tissue, cell), or subject. Another aspect of the disclosure relates to methods of inhibiting the transcription of a gene (e.g., a gene controlled or regulated by a transcription factor (e.g., TEAD, such as TEAD1, TEAD2, TEAD3, TEAD4)) in a biological sample (e.g., tissue, cell), or subject.

In certain embodiments, the methods described herein include administering to a subject or contacting a biological sample with an effective amount of a compound described herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the methods described herein include administering to a subject or contacting a biological sample with an effective amount of a compound described herein, or a pharmaceutically acceptable salt thereof, or a pharmaceutical composition thereof. In certain embodiments, the compound is contacted with a biological sample. In certain embodiments, the compound is administered to a subject. In certain embodiments, the compound is administered in combination with one or more additional pharmaceutical agents described herein. The additional pharmaceutical agent may be an anti-proliferative agent. In certain embodiments, the additional pharmaceutical agent is an anti-cancer agent. The additional pharmaceutical agent may also be a transcription factor inhibitor. In certain embodiments, the additional pharmaceutical agent is a transcription factor inhibitor (e.g., inhibitor of EGFR and/or MEK). In certain embodiments, the additional pharmaceutical agent comprises an inhibitor of EGFR and an inhibitor of MEK. In certain embodiments, the additional pharmaceutical agent is a binder or inhibitor of TEAD (e.g., TEAD1, TEAD2, TEAD3, TEAD4)). In certain embodiments, the additional pharmaceutical agent is a binder or inhibitor of TEAD. In certain embodiments, the additional pharmaceutical agent is a binder or inhibitor of TEAD1. In certain embodiments, the additional pharmaceutical agent is a binder or inhibitor of TEAD2. In certain embodiments, the additional pharmaceutical agent is a binder or inhibitor of TEAD3. In certain embodiments, the additional pharmaceutical agent is a binder or inhibitor of TEAD4. In certain embodiments, the additional pharmaceutical agent is a selective binder of TEAD. In certain embodiments, the additional pharmaceutical agent is a selective binder of TEAD1. In certain embodiments, the additional pharmaceutical agent is a selective binder of TEAD2. In certain embodiments, the additional pharmaceutical agent is a selective binder of TEAD3. In certain embodiments, the additional pharmaceutical agent is a selective binder of TEAD4. In certain embodiments, the additional pharmaceutical agent is a selective inhibitor of TEAD. In certain embodiments, the additional pharmaceutical agent is a selective inhibitor of TEAD1. In certain embodiments, the additional pharmaceutical agent is a selective inhibitor of TEAD2. In certain embodiments, the additional pharmaceutical agent is a selective inhibitor of TEAD3. In certain embodiments, the additional pharmaceutical agent is a selective inhibitor of TEAD4. In certain embodiments, the additional pharmaceutical agent is a non-selective binder of TEAD1. In certain embodiments, the additional pharmaceutical agent is a non-selective binder of TEAD2. In certain embodiments, the additional pharmaceutical agent is a non-selective binder of TEAD3. In certain embodiments, the additional pharmaceutical agent is a non-selective binder of TEAD4. In certain embodiments, the additional pharmaceutical agent is a non-selective inhibitor of TEAD. In certain embodiments, the additional pharmaceutical agent is a non-selective inhibitor of TEAD1. In certain embodiments, the additional pharmaceutical agent is a non-selective inhibitor of TEAD2. In certain embodiments, the additional pharmaceutical agent is a non-selective inhibitor of TEAD3. In certain embodiments, the additional pharmaceutical agent is a non-selective inhibitor of TEAD4. In certain embodiments, the additional pharmaceutical agent is a selective inhibitor of EGFR. In certain embodiments, the additional pharmaceutical agent is a selective inhibitor of MEK. In certain embodiments, the additional pharmaceutical agent is a non-selective inhibitor of EGFR and/or MEK. In certain embodiments, the additional pharmaceutical agent includes an anti-cancer agent (e.g., chemotherapeutics), anti-inflammatory agent, steroids, immunosuppressant, radiation therapy, or other agents. In certain embodiments, the additional pharmaceutical agent is an anti-proliferative agent. In certain embodiments, the additional pharmaceutical agent is an inhibitor of a kinase. In certain embodiments, the additional pharmaceutical agent is a non-selective inhibitor of a kinase. In certain embodiments, the additional pharmaceutical agent is an immunotherapy agent (e.g., PD1 inhibitor, PDL1 inhibitor). In certain embodiments, the additional pharmaceutical agent is an immune checkpoint inhibitor.

In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In some embodiments, the additional pharmaceutical agent is etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. Exemplary chemotherapeutic agents include alkylating agents such as nitrogen mustards, ethylenimines, methylmelamines, alkyl sulfonates, nitrosuoureas, and triazenes; antimetabolites such as folic acid analogs, pyrimidine analogs, in particular fluorouracil and cytosine arabinoside, and purine analogs; natural products such as vinca alkaloids epi-podophyllotoxins, antibiotics, enzymes, and biological response modifiers; and miscellaneous products such as platinum coordination complexes, anthracenedione, substituted urea such as hydroxyurea, methyl hydrazine derivatives, and adrenocorticoid suppressant, including ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. Exemplary chemotherapeutic agents also include anthracycline antibiotics, actinomycin D, plicamycin, puromycin, gramicidin D, paclitaxel, colchicine, cytochalasin B, emetine, maytansine, amsacrine, cisplatin, carboplatin, mitomycin, altretamine, cyclophosphamide, lomustine, and carmustine. In certain embodiments, a pharmaceutical composition described herein further comprises a combination of the additional pharmaceutical agents described herein.

The inventive compounds or pharmaceutical compositions thereof used with an additional pharmaceutical agent may synergistically augment inhibition (e.g., increase the degree of inhibition) of a transcription factor (e.g., TEAD, such as TEAD1, TEAD2, TEAD3, TEAD4) induced by the additional pharmaceutical agent(s) in the biological sample or subject. For example, use of the inventive compounds or pharmaceutical compositions thereof with an additional pharmaceutical agent may increase the degree of inhibition of a transcription factor (e.g., TEAD, such as TEAD1, TEAD2, TEAD3, TEAD4) compared to the degree of inhibition of a transcription factor (e.g., TEAD, such as TEAD1, TEAD2, TEAD3, TEAD4) induced by the additional pharmaceutical agent alone. Thus, the combination of the inventive compounds or compositions and the additional pharmaceutical agent(s) may be useful in treating proliferative diseases resistant to a treatment using the additional pharmaceutical agent(s) without the inventive compounds or compositions.

In some embodiments, the activity of a transcription factor (e.g., TEAD, such as TEAD1, TEAD2, TEAD3, TEAD4) is non-selectively inhibited by the compounds or pharmaceutical compositions described herein. In some embodiments, the activity of the transcription factor (e.g., TEAD, such as TEAD1, TEAD2, TEAD3, TEAD4) being inhibited is selectively inhibited by the compounds or pharmaceutical compositions described herein, compared to the activity of a different protein (e.g., a different transcription factor (e.g., TEAD, such as TEAD1, TEAD2, TEAD3, TEAD4)). In certain embodiments, the activity of a transcription factor (e.g., TEAD, such as TEAD1, TEAD2, TEAD3, TEAD4)) is selectively inhibited by a compound or pharmaceutical composition described herein, compared to the activity of a different protein. In certain embodiments, the activity of TEAD1 is selectively inhibited by a compound or pharmaceutical composition described herein, compared to the activity of another TEAD (e.g., TEAD2, TEAD3, TEAD4). In certain embodiments, the activity of TEAD2 is selectively inhibited by a compound or pharmaceutical composition described herein, compared to the activity of another TEAD (e.g., TEAD1, TEAD3, TEAD4). In certain embodiments, the activity of TEAD3 is selectively inhibited by a compound or pharmaceutical composition described herein, compared to the activity of another TEAD (e.g., TEAD1, TEAD2, TEAD4). In certain embodiments, the activity of TEAD4 is selectively inhibited by a compound or pharmaceutical composition described herein, compared to the activity of another TEAD (e.g., TEAD1, TEAD2, TEAD3).

The selectivity of a compound or pharmaceutical composition described herein in inhibiting the activity of a transcription factor (e.g., TEAD, such as TEAD1, TEAD2, TEAD3, TEAD4) over a different protein (e.g., a different transcription factor (e.g., TEAD)) may be measured by the quotient of the IC₅₀ value of the compound or pharmaceutical composition in inhibiting the activity of the different protein over the IC₅₀ value of the compound or pharmaceutical composition in inhibiting the activity of the transcription factor (e.g., TEAD). The selectivity of a compound or pharmaceutical composition described herein for a protein transcription factor (e.g., TEAD) over a different protein may also be measured by the quotient of the K_{d} value of an adduct of the compound or pharmaceutical composition and the different protein over the K_{d} value of an adduct of the compound or pharmaceutical composition and the transcription factor (e.g., TEAD). In certain embodiments, the selectivity is at least 2-fold, at least 3-fold, at least 5-fold, at least 10-fold, at least 30-fold, at least 100-fold, at least 300-fold, at least 1,000-fold, at least 3,000-fold, at least 10,000-fold, at least 30,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

In certain embodiments, a kit described herein includes a first container comprising a compound or pharmaceutical composition described herein. In certain embodiments, a kit described herein is useful in treating and/or preventing a disease, such as a proliferative disease (e.g., cancers (e.g., carcinoma, sarcoma); lung cancer, breast cancer, liver cancer, pancreatic cancer, gastric cancer, ovarian cancer, colon cancer, colorectal cancer, skin cancer, esophageal cancer)), inflammatory disease (e.g., fibrosis), or autoimmune disease (e.g., sclerosis), in a subject in need thereof, inhibiting the activity of a transcription factor (e.g., TEAD, such as TEAD1, TEAD2, TEAD3, TEAD4), and/or inhibiting the transcription of a gene (e.g., a gene controlled or regulated by a transcription factor (e.g., TEAD, such as TEAD1, TEAD2, TEAD3, TEAD4)) in a subject, and/or biological sample (e.g., tissue, cell).

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a proliferative disease in a subject in need thereof, preventing a disease, such as a proliferative disease, inflammatory disease, autoimmune disease in a subject in need thereof, inhibiting the activity of a transcription factor (e.g., TEAD, such as TEAD1, TEAD2, TEAD3, TEAD4)) in a subject and/or biological sample (e.g., tissue, cell), and/or inhibiting the transcription of a gene (e.g., a gene controlled or regulated by a transcription factor (e.g., TEAD, such as TEAD1, TEAD2, TEAD3, TEAD4)). A kit described herein may include one or more additional pharmaceutical agents described herein as a separate composition.

### EXAMPLES

In order that the present disclosure may be more fully understood, the following examples are set forth. The examples described in this application are offered to illustrate the compounds, pharmaceutical compositions, and methods provided herein and are not to be construed in any way as limiting their scope. The compounds provided herein can be prepared from readily available starting materials using the following general methods and procedures or methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Compounds of Formulas (I-A), (I-B), or (II) may be prepared using synthetic schemes and procedures known by one of ordinary skill in the art.

### Example 1. Synthesis of Exemplary TEAD Inhibitor Compounds

Compounds of Formulas (I-A), (I-B), or (II) may be prepared in view of the following synthetic schemes, and by using synthetic schemes and procedures recognized by one of ordinary skill in the art.

Unless otherwise noted, reagents and solvents were obtained from commercial suppliers and were used without further purification. ¹H NMR spectra were recorded on 500 MHz (Varian AS600), and chemical shifts are reported in parts per million (ppm, δ) downfield from tetramethylsilane (TMS). Coupling constants (J) are reported in Hz. Spin multiplicities are described as s (singlet), br (broad singlet), d (doublet), t (triplet), q (quartet), and m (multiplet). Mass spectra were obtained on a Waters Micromass ZQ instrument. Preparative HPLC was performed on a Waters Sunfire C18 column (19 mm × 50 mm, 5 µM) using a gradient of 15-95% methanol in water containing 0.05% trifluoroacetic acid (TFA) over 22 min (28 min run time) at a flow rate of 20 mL/min. Purities of assayed compounds were in all cases greater than 95%, as determined by reverse-phase HPLC analysis.

### Scheme 1: Synthesis of MYF-1-37

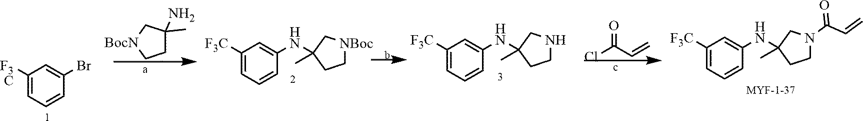

^{a}Reagents and conditions: (a) Pd(OAc)2, XPhos, NaOtBu, toluene, 100° C. ; (b) HCl/dioxane, MeOH; (c) DIEA, MeCN, 0° C.

### Tert-butyl 3-methyl-3-((3-(trifluoromethyl)phenyl)amino)pyrrolidine-1-carboxylate (2)

To a solution of 1-bromo-3-(trifluoromethyl)benzene (223 mg, 1.0 mmol) and tert-butyl 3-amino-3-methylpyrrolidine-1-carboxylate (200 mg, 1.0 mmol) in 5 mL of toluene was added Pd(OAc)₂ (22 mg, 0.1 mmol), XPhos (58 mg, 0.1 mmol) and NaOtBu (192 mg, 2 mmol) under N₂. The mixture was stirred at 100° C. overnight. The mixture was filtered. The filtrate was concentrated in vacuo, then purified by flash chromatography on silica gel (hexane: ethyl acetate = 4:1) to provide compound 2 (240 mg, 70%). LC/MS (ESI) m/z = 345 (M + H)+.

### 3-methyl-N-(3-(trifluoromethyl)phenyl)pyrrolidin-3-amine (3)

To a solution of tert-butyl 3-methyl-3-((3-(trifluoromethyl)phenyl)amino)pyrrolidine-1-carboxylate (240 mg, 0.7 mmol) in 3 mL of methanol was added 4N HCl/dioxane (1 mL) solution. The result solution was stirred at room temperature for 1 h, and then concentrated in vacuo to obtain the product as HCl salt, which was used into next step without any purification. LC/MS (ESI) m/z = 245 (M + H)+.

### 1-Methyl-3-((trifluoromethyl)phenyl)amino)pyrrolidin-1-yl)prop-2-en-1-one (MYF-1-37)

To a solution of 3-methyl-N-(3-(trifluoromethyl)phenyl)pyrrolidin-3-amine (28 mg, 0.1 mmol) and DIEA (33 uL, 0.2 mmol) in 1 mL of acetonitrile was added acryloyl chloride dropwise at 0° C. until the reaction completed. The mixture was diluted with dichloromethane, washed with 1 N NaHCO3 solution and brine. The organic layer was dried over sodium sulfate, concentrated in vacuo and then purified by prep-HPLC (MeOH/H2O, 0-100%) to provide the title compound (23.4 mg, 79%). LC/MS (ESI) m/z = 299 (M + H)+. 1H NMR (500 MHz, DMSO-d6) δ 7.28 (t, J = 8.0 Hz, 1H), 6.98 - 6.87 (m, 2H), 6.83 (d, J = 7.4 Hz, 1H), 6.56 (ddd, J = 18.1, 16.8, 10.3 Hz, 1H), 6.21 (d, J = 4.3 Hz, 1H), 6.12 (ddd, J = 16.8, 6.6, 2.4 Hz, 1H), 5.66 (ddd, J = 10.3, 8.8, 2.4 Hz, 1H), 3.87 - 3.73 (m, 1H), 3.71 - 3.59 (m, 1.5H), 3.53 - 3.43 (m, 1H), 3.39 (d, J = 12.3 Hz, 0.5H), 2.36 - 2.16 (m, 1H), 2.08 - 1.85 (m, 1H), 1.42 (s, 3H).

### MYF37 Docking to TEAD2

MYF-01-037-02 was docked into TEAD2 crystal structure (pdbcode: 5HGU) using Glide covalent docking program (version 2019 release 1). The Cys380 was defined as the reactive residue for Michael addition reaction. Default parameter values were used for docking calculation. Prior to docking, the protein structure was processed and energy optimized using protein preparation protocol in Schrodinger suite software.

### MYF37 Competition Pulldown

MDA-MB-231 cells were incubated for 6 hours with 10, 25 and 50 µM MYF-01-37, followed by cell lysis. The lysates were subjected to pulldown with 50 µM biotinylated MYF-01-37 overnight, and the amount of TEAD pulled down was analyzed by western blotting.

### Mass Spectrometry Analysis

TEAD2 protein was incubated with DMSO or a 20-fold molar excess of MYF-01-37 for 6 hours at 37° C. Reactions were then analyzed by LC-MS using a Shimadzu autosampler and LC (Marlborough, MA) coupled to an LTQ ion trap mass spectrometer (ThermoFisher Scientific, San Jose, CA). Protein was injected onto a self packed column (0.5 mm I.D., packed 5 cm POROS 50R2 from Applied Biosystems, Framingham, MA), desalted for 4 minutes with 100% A (A=0.2 M acetic acid in water), eluted with a gradient (0-100% B in 1 minute; A=0.2 M acetic acid in water, B=0.2 M acetic acid in acetonitrile), and introduced to the mass spectrometer by electrospray ionization (spray voltage=4.5 kV). The mass spectrometer acquired full scan MS data (m/z 300-2000). Mass spectra were deconvoluted using MagTran version 1.03b2 (Zhang and Marshall, 1998).

To identify the site of modification, labeled protein was diluted 1:1 with 100 mM ammonium bicarbonate, reduced with 10 mM DTT at 56° C. for 30 minutes, alkylated with 22.5 mM IAA for 30 minutes at room temperature, and then digested with trypsin overnight at 37° C. Tryptic peptides were desalted by C18 (SOLA, ThermoFisher Scientific), dried by vacuum centrifugation, reconstituted in 5% MeCN, 0.1% trifluoroacetic acid, and analyzed by nanoLC-ion mobility MS/MS using a NanoAcquity UPLC system (Waters Corp., Milford, MA) interfaced to a timsTOF Pro mass spectrometer (Bruker, Billerica, MA). Peptides were injected onto a self-packed pre-column (4 cm POROS10R2, Applied Biosystems), resolved on an analytical column (30 µm I.D. × 50 cm Monitor C18, Orochem, Naperville, IL; 10-60% B in 40 minutes; A = 0.2 M acetic acid in water, B = 0.2 M acetic acid in acetonitrile) and introduced to the mass spectrometer by electrospray ionization using a captive spray ion source (spray voltage = 2 kV). The mass spectrometer collected ion mobility MS spectra over a mass range of m/z 100-1700 and 1/k0 of 0.6 to 1.6, and then performed 10 cycles of PASEF MS/MS with a target intensity of 20 k and a threshold of 250. Active exclusion was enabled with a release time of 0.4 minutes. Raw data was converted to .mgf using the tdf to mgf converter (Bruker), and searched using Mascot 2.6.1 against a forward reversed human refseq database (NCBI). Search parameters specified a precursor mass tolerance of 20 ppm, a product ion tolerance of 50 mmu, fixed carbamidomethylation of cysteine, and variable oxidation of methionine as well as variable MYF-1-37 modification of cysteine. Search results were downloaded and converted to xls using multiplierz software (Alexander et al., 2017) , and peptide fragment ions were assigned using using mzStudio (Ficarro et al., 2017). Inhibitor related fragment ions were assigned as described (Ficarro et al., 2016).

Compounds of Formula (I-A) may be prepared using the synthetic scheme shown below (Scheme 1A) and procedures known by one of ordinary skill in the art.

### Scheme 1A. Preparation of Compounds of Formula (I-A)

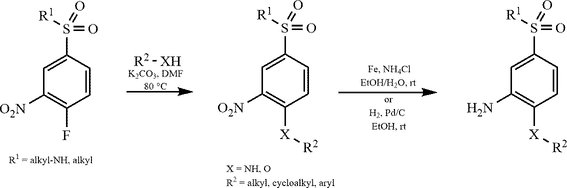

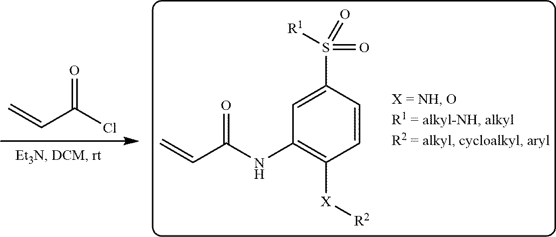

Compounds of Formula (I-B) may be prepared using the synthetic scheme shown below (Scheme 2) and procedures known by one of ordinary skill in the art.

### Scheme 2. Preparation of Compounds of Formula (I-B)

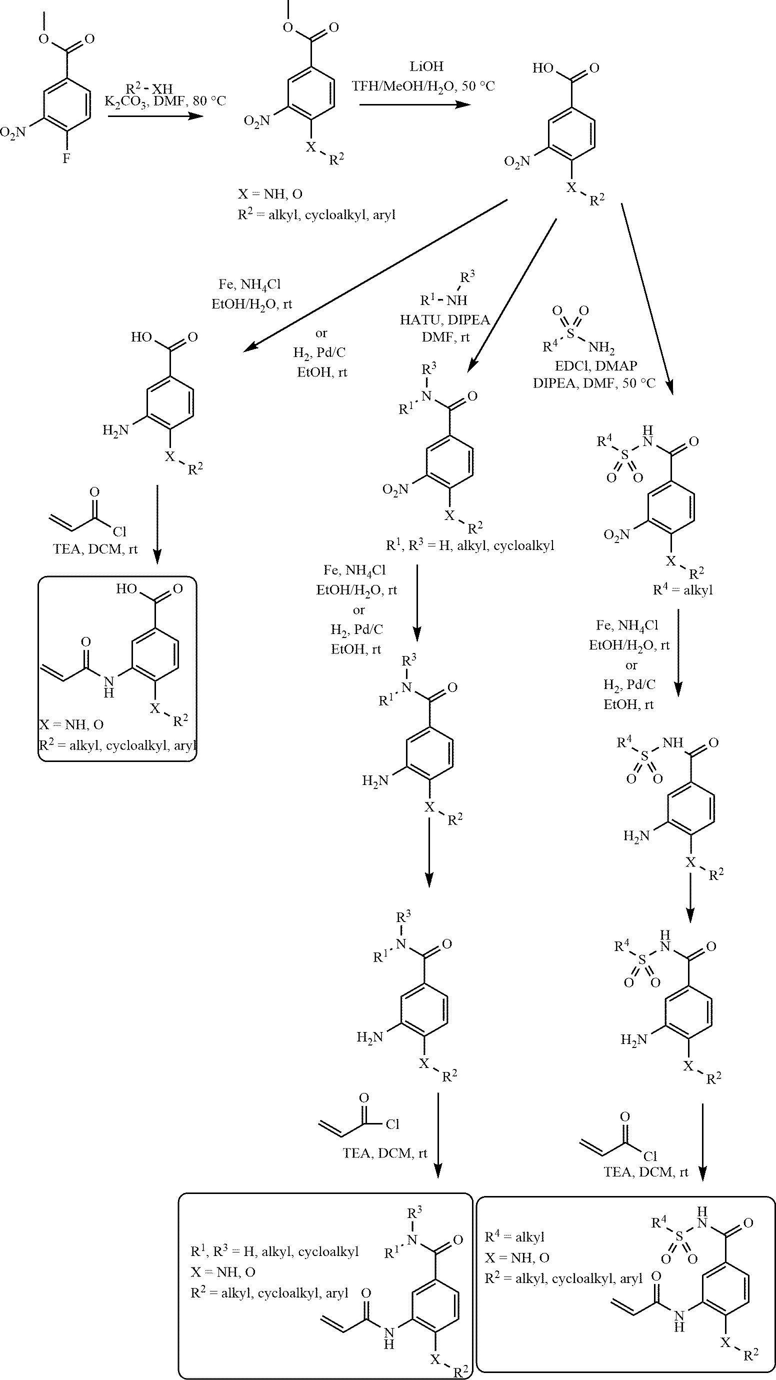

Compounds of Formula (II) may be prepared using the synthetic scheme shown below (Scheme 3) and procedures recognized by one of ordinary skill in the art.

### Scheme 3. Preparation of Compounds of Formula (II)

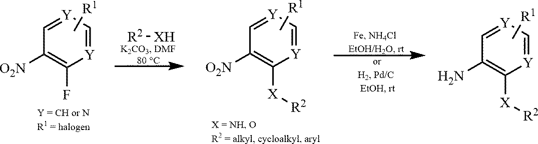

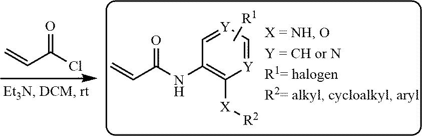

### Synthesis of Exemplary Compounds

### I-a-01

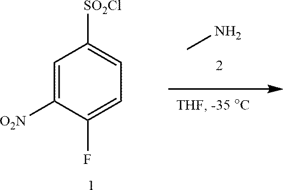

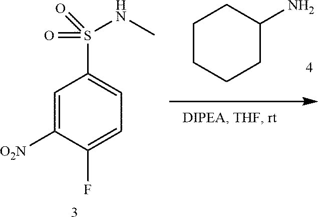

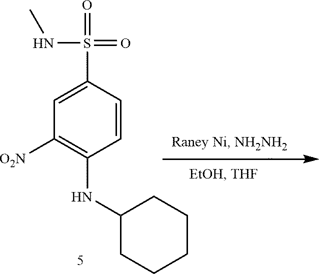

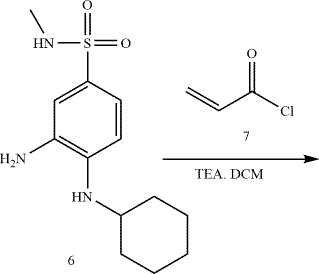

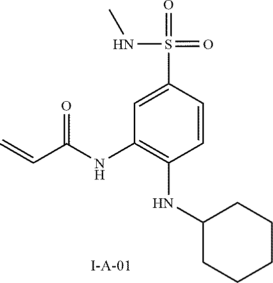

### Step 1: Synthesis of 4-fluoro-N-methyl-3-nitrobenzenesulfonamide (Compound 3)

To the mixture of 4-fluoro-3-nitrobenzene-1-sulfonyl chloride (2 g, 8.4 mmol) in THF (80 mL) was added Et₃ N (5.08 g, 50.4 mmol), the mixture was stirred at -35° C. under N₂ for 10 min., and then the solution of methylamine in THF (1 M, 10 mL, 10 mmol) was added dropwise, the mixture was stirred at -35° C. for 1 hour, diluted with EtOAc (100 mL), washed with brine (50 mL), dried over anhydrous Na₂SO₄, filtered and concentrated to leave the crude product (2 g) as a yellow oil, which was purified by flash column chromatography on silica gel (ethyl acetate in petroleum ether = 50% v/v) to obtain the target compound 3 (1.33 g, yield 68.2%) as solid. ¹H NMR (400 MHz, DMSO-d₆) δ (ppm) 8.47 (dd, J = 7.0, 2.3 Hz, 1H), 8.18 (ddd, J = 8.7, 4.0, 2.4 Hz, 1H), 7.93 - 7.80 (m, 2H), 2.48 (d, J = 4.9 Hz, 3H).

### Step 2: Synthesis of 4-(cyclohexylamino)-N-methyl-3-nitrobenzenesulfonamide (Compound 5)

To the mixture of 4-fluoro-N-methyl-3-nitrobenzenesulfonamide (400 mg, 1.71 mmol) in THF (40 mL) was added cyclohexylamine (168.7 mg, 1.71 mmol) and DIPEA (655.5 mg, 5.12 mmol). The mixture was stirred at rt under N₂ for 3 h., diluted with EtOAc (100 mL), washed with brine (50 mL), dried over anhydrous Na₂SO₄, concentrated and purified by flash column chromatography on silica gel (ethyl acetate in petroleum ether = 20% v/v) to afford the target compound 5 (510 mg, yield 95.5%) as pale yellow solid. LC-MS (ESI) m/z: 314 [M+H]⁺.

### Step 3: Synthesis of 3-amino-4-(cyclohexylamino)-N-methylbenzenesulfonamide (Compound 6)

To the mixture of 4-(cyclohexylamino)-N-methyl-3-nitrobenzenesulfonamide (250 mg, 0.8 mmol) and Raney nickel (40 mg, 0.1 mmol) in EtOH (10 mL) and THF (10 mL) was added N₂H₄—H₂O (100 mg, 2.0 mmol). The mixture was stirred at rt for 1 h and filtered, the filtrate was concentrated and purified by flash column chromatography on silica gel (ethyl acetate in petroleum ether = 40% v/v) to obtain the target compound 6 (200 mg, yield 88.5%) as solid. LC-MS (ESI) m/z: 284 [M+H]⁺.

### Step 4: Synthesis of N-(2-(cyclohexylamino)-5-(N-methylsulfamoyl)phenyl)acrylamide (Compound I-A-01)

To the mixture of 3-amino-4-(cyclohexylamino)-N-methylbenzenesulfonamide (150 mg, 0.51 mmol) and Et₃ N (102 mg, 1.02 mmol) in DCM (15 mL) was added acryloyl chloride (48 mg, 0.51 mmol). The mixture was stirred at 0° C. under N₂ for 1 h, and concentrated under vacuum, the residue was purified by prep-HPLC (MeCN/H₂O/TFA) to obtain the target compound I-A-01 (92 mg, yield 51.6%) as solid. LC-MS (ESI) m/z: 338.2 [M+H]⁺. ¹H NMR (400 MHz, DMSO-d₆) δ (ppm) 9.47 (s, 1H), 7.71 (s, 1H), 7.40 (d, J = 8.7 Hz, 1H), 7.07 (d, J = 5.0 Hz, 1H), 6.81 (d, J = 8.8 Hz, 1H), 6.51 (dd, J = 17.0, 10.2 Hz, 1H), 6.26 (d, J = 16.9 Hz, 1H), 5.78 (d, J = 9.9 Hz, 1H), 5.40 (d, J = 7.5 Hz, 1H), 2.36 (d, J = 5.0 Hz, 3H), 1.95 (d, J = 10.8 Hz, 2H), 1.72 (d, J = 10.0 Hz, 2H), 1.62 (d, J = 11.6 Hz, 1H), 1.44-1.10 (m, 5H).

### I-a-02

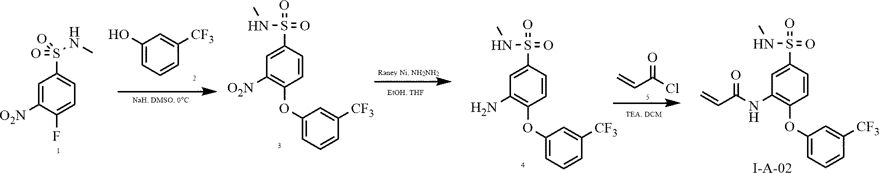

### Step 1: Synthesis of N-methyl-3-nitro-4-(3-(trifluoromethyl)phenoxy)benzenesulfonamide (Compound 3)

To the solution of 4-fluoro-N-methyl-3-nitrobenzenesulfonamide (300 mg, 1.28 mmol) in DMSO (15 mL) was added NaH (60%, 61.5 mg, 1.53 mmol), the mixture was stirred at 0° C. under N₂ for 0.5 h., and then 3-(trifluoromethyl)phenol (207 mg, 1.29 mmol) was added. The mixture was stirred at rt overnight, diluted with EtOAc (100 mL), washed with brine (50 mL), dried over anhydrous Na₂SO₄, concentrated and purified by flash column chromatography on silica gel (ethyl acetate in petroleum ether = 50% v/v) to obtain the target compound 3 (200 mg, yield 41.4%) as yellow oil. LC-MS (ESI) m/z: 377 [M+H]⁺.

### Step 2: Synthesis of 3-amino-N-methyl-4-(3-(trifluoromethyl)phenoxy)benzenesulfonamide (Compound 4)

To the mixture of N-methyl-3-nitro-4-(3-(trifluoromethyl)phenoxy) benzenesulfonamide (150 mg, 0.4 mmol) and Raney nickel (16 mg, 0.04 mmol) in EtOH (10 mL) and THF (10 mL) was added N₂H₄—H₂O (20 mg, 0.4 mmol). The mixture was stirred at rt for 1 h and filtered, the filtrate was concentrated at reduced pressure to leave the crude compound 4 (130 mg, crude) as oil. LC-MS (ESI) m/z: 347 [M+H]⁺.

### Step 3: Synthesis of N-(5-(N-methylsulfamoyl)-2-(3-(trifluoromethyl)phenoxy)phenyl)acrylamide (Compound I-A-02)

To the mixture of 3-amino-N-methyl-4-(3-(trifluoromethyl)phenoxy)benzenesulfonamide (100 mg, 0.29 mmol) and Et₃ N (58 mg, 0.58 mmol) in DCM (15 mL) was added acryloyl chloride (26 mg, 0.29 mmol). The mixture was stirred at 0° C. under N₂ for 1 h, and then concentrated under vacuum, the residue was purified by prep-HPLC (MeCN/H₂O/TFA) to obtain the target compound I-A-02 (11 mg, yield 9.6%) as solid. LC-MS (ESI) m/z: 401 [M+H]⁺. ¹H NMR (400 MHz, CD₃OD) δ (ppm) 8.58 (d, J = 2.1 Hz, 1H), 7.61 - 7.48 (m, 2H), 7.43 (d, J = 7.6 Hz, 1H), 7.34 (s, 1H), 7.25 (d, J = 8.1 Hz, 1H), 6.94 (d, J = 8.6 Hz, 1H), 6.46 (dd, J = 17.0, 10.2 Hz, 1H), 6.29 (dd, J = 17.0, 1.7 Hz, 1H), 5.69 (dd, J = 10.2, 1.7 Hz, 1H), 4.53 (s, 1H), 2.48 (s, 3H).

### I-b-01

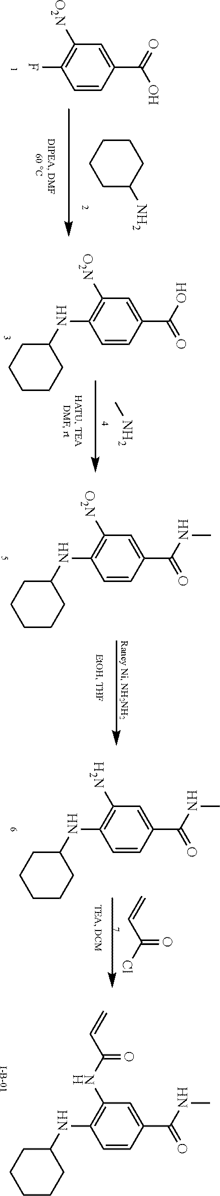

### Step 1: Synthesis of 4-(cyclohexylamino)-3-nitrobenzoic Acid (Compound 3)

The mixture of 4-fluoro-3-nitrobenzoic acid (1 g, 5.4 mmol), cyclohexanamine (534 mg, 5.4 mmol) and DIPEA (2.1 g, 16.2 mmol) in DMF (25 mL) re was stirred at 60° C. under N₂ for 3 h. The resulting suspension was filtered, the cake was dried under vacuum to obtain the target compound 3 (1 g, crude) as solid. LC-MS (ESI) m/z: 265 [M+H]⁺.

### Step 2: Synthesis of 4-(cyclohexylamino)-N-methyl-3-nitrobenzamide (Compound 5)

To the solution of 4-(cyclohexylamino)-3-nitrobenzoic acid (1.5 g, 4.4 mmol) in DMF (20 mL) was added methylamine solution in THF (2.1 mL, 2 mol/L, 4.4 mmol), and then HATU (2 g, 5.28 mmol) and Et₃N (885 mg, 8.8 mmol) was added. The mixture was stirred at rt for 3 h., diluted with EtOAc (100 mL), washed with brine (50 mL), dried over anhydrous Na₂SO₄, filtered, concentrated and purified by flash column chromatography on silica gel (ethyl acetate in petroleum ether = 20% v/v) to obtain the target compound 5 (300 mg, yield 19.1%) as solid. LC-MS (ESI) m/z: 278 [M+H]⁺.

### Step 3: Synthesis of 3-amino-4-(cyclohexylamino)-N-methylbenzamide (Compound 6)

To the mixture of 4-(cyclohexylamino)-N-methyl-3-nitrobenzamide (240 mg, 0.88 mmol) and Raney nickel (32 mg, 0.08 mmol) in EtOH (10 mL) and THF (10 mL) was added N₂H₄-H₂O (200 mg, 4.0 mmol). The mixture was stirred at rt for 1 h and filtered, the filtrate was concentrated at reduced pressure to leave the crude compound 6 (100 mg, crude) as oil. LC-MS (ESI) m/z: 248 [M+H]⁺.

### Step 4: Synthesis of 3-acrylamido-4-(cyclohexylamino)-N-methylbenzamide (Compound I-B-01)

To the mixture of 3-amino-4-(cyclohexylamino)-N-methylbenzamide (100 mg, 0.4 mmol) and Et₃ N (80 mg, 0.8 mmol) in DCM (10 mL) was added acryloyl chloride (36 mg, 0.4 mmol). The mixture was stirred at 0° C. for 15 minutes, and then purified directly by prep-HPLC (MeCN/H₂O/TFA) to obtain the target compound I-B-01 (38 mg, yield 31.4%) as solid. LC-MS (ESI) m/z: 302.3 [M+H]⁺. ¹H NMR (400 MHz, DMSO-d₆) δ (ppm) 9.45 (s, 1H), 8.07 (d, J = 4.4 Hz, 1H), 7.73 (d, J = 1.5 Hz, 1H), 7.56 (d, J = 8.5 Hz, 1H), 6.69 (d, J = 8.7 Hz, 1H), 6.50 (dd, J = 17.0, 10.2 Hz, 1H), 6.23 (dd, J = 17.0, 1.8 Hz, 1H), 5.76 (dd, J = 10.2, 1.6 Hz, 1H), 5.08 (d, J = 7.6 Hz, 1H), 3.34 - 3.29 (m, 1H), 2.72 (d, J = 4.4 Hz, 3H), 1.93 (d, J = 10.2 Hz, 2H), 1.79 - 1.51 (m, 3H), 1.43-1.11 (m, 5H).

### I-b-02

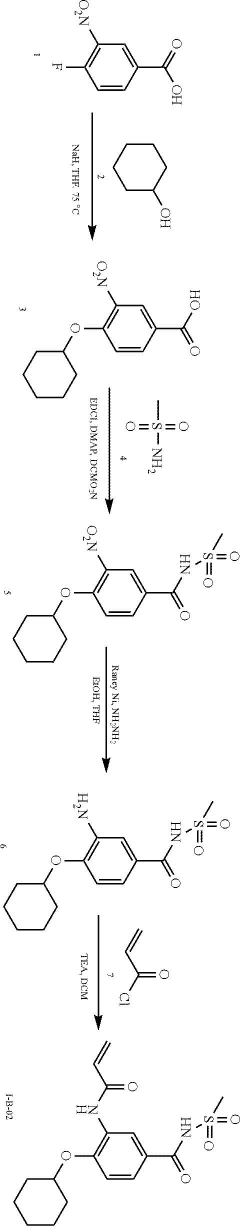

### Step 1: Synthesis of 4-(cyclohexyloxy)-3-nitrobenzoic Acid (Compound 3)

To the solution of cyclohexanol (1.08 g, 10.8 mmol) in THF (50 mL) was added NaH (60%, 520 mg, 13.0 mmol), the mixture was stirred at 0° C. under N₂ for 0.5 h., and then 4-fluoro-3-nitrobenzoic acid (2 g, 10.8 mmol) was added. The mixture was stirred at 75° C. for 4 h. After cooled down to rt the mixture was diluted with EtOAc (100 mL), washed with brine (50 mL), dried over anhydrous Na₂SO₄, filtered and concentrated at reduced pressure to leave the crude compound 3 (2 g, crude) as oil. LC-MS (ESI) m/z: no MS.

### Step 2: Synthesis of 4-(cyclohexyloxy)-N-(methylsulfonyl)-3-nitrobenzamide (Compound 5)

To the solution of 4-(cyclohexyloxy)-3-nitrobenzoic acid (2 g, 7.55 mmol) in DCM (100 mL) was added EDCI (2.15 g, 11.32 mmol) and DMAP (2.11 g, 17.35 mmol). The mixture was stirred at rt for 10 min., and then methanesulfonamide (1.06 g, 11.32 mmol) was added. The mixture was stirred at rt overnight, concentrated and purified by flash column chromatography on silica gel (methylene chloride in methanol = 20% v/v) to obtain the target compound 5 (1.22 g, yield 47.2%) as solid. LC-MS (ESI) m/z: 343 [M+H]⁺.

### Step 3: Synthesis of 3-amino-4-(cyclohexyloxy)-N-(methylsulfonyl)benzamide (Compound 6)

To the mixture of 4-(cyclohexyloxy)-N-(methylsulfonyl)-3-nitrobenzamide (500 mg, 1.4 mmol) and Raney nickel (60 mg, 0.2 mmol) in EtOH (25 mL) and THF (25 mL) was added N₂H₄•H₂O (200 mg, 4.0 mmol). The mixture was stirred at rt for 1 h and filtered, the filtrate was concentrated at reduced pressure to leave the crude compound 6 (200 mg, crude) as oil. LC-MS (ESI) m/z: 313 [M+H]⁺.

### Step 4: Synthesis of 3-acrylamido-4-(cyclohexyloxy)-N-(methylsulfonyl)benzamide (Compound I-B-02)

To the mixture of 3-amino-4-(cyclohexyloxy)-N-(methylsulfonyl)benzamide (150 mg, 0.45 mmol) and Et₃N (90 mg, 0.90 mmol) in DCM (15 mL) was added acryloyl chloride (39 mg, 0.45 mmol). The mixture was stirred at 0° C. for 1 h., and then purified by prep-HPLC to obtain the target compound I-B-02 (25 mg, yield 14.2%) as solid. LC-MS (ESI) m/z: 367 [M+H]⁺. ¹H NMR (400 MHz, DMSO-d₆) δ (ppm) 9.15 (s, 1H), 8.47 (s, 1H), 8.15 (s, 1H), 7.69 (d, J = 8.3 Hz, 1H), 7.12 (dd, J = 45.1, 8.6 Hz, 1H), 6.65 (dd, J = 16.5, 10.4 Hz, 1H), 6.24 (d, J = 17.1 Hz, 1H), 5.73 (d, J = 10.0 Hz, 1H), 4.44 (br, 1H), 2.94 (d, J = 26.6 Hz, 3H), 1.98-1.94 (m, 2H), 1.81-1.65 (m, 2H), 1.61-1.44 (m, 3H), 1.43 - 1.18 (m, 3H).

### I-b-03

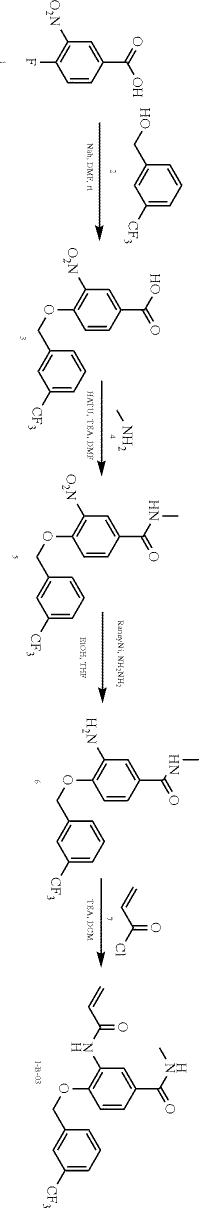

### Step 1: Synthesis of 3-nitro-4-(3-(trifluoromethyl)benzyloxy)benzoic Acid (Compound 3)

To the solution of 4-fluoro-3-nitrobenzoic acid (1 g, 5.4 mmol) in DMF (20 mL) was added NaH (60%, 260 mg, 6.5 mmol), the mixture was stirred at 0° C. under N₂ atmosphere for 0.5 h., and then (3-(trifluoromethyl)phenyl)methanol (1.52 mg, 8.6 mmol) was added. The mixture was stirred at rt for 2 h., diluted with EtOAc (100 mL), washed with brine (50 mL), dried over anhydrous Na₂SO₄, filtered and concentrated at reduced pressure to leave the target compound 3 (600 mg, yield 32.6%) as solid. LC-MS (ESI) m/z: 365 [M+Na]⁺.

### Step 2: Synthesis of N-methyl-3-nitro-4-(3-(trifluoromethyl)benzyloxy)benzamide (Compound 5)

To the solution of 3-nitro-4-(3-(trifluoromethyl)benzyloxy)benzoic acid (500 mg, 1.45 mmol) in DMF (15 mL) was added methylamine solution in THF (2 M, 0.7 mL, 1.4 mmol), and then HATU (668.5 mg, 1.75 mmol) and Et₃ N (295 mg, 2.9 mmol) was added. The mixture was stirred at rt for 1 h., concentrated and purified by flash column chromatography on silica gel (ethyl acetate in petroleum ether = 20% v/v) to obtain the target compound 5 (500 mg, yield 96.3%) as solid. LC-MS (ESI) m/z: 355 [M+H]⁺.

### Step 3: Synthesis of 3-amino-N-methyl-4-(3-(trifluoromethyl)benzyloxy)benzamide (Compound 6)

To the mixture of N-methyl-3-nitro-4-(3-(trifluoromethyl)benzyloxy)benzamide (500 mg, 1.4 mmol) and Raney nickel (60 mg, 0.2 mmol) in EtOH (15 mL) and THF (15 mL) was added N₂H₄•H₂O (200 mg, 4.0 mmol). The mixture was stirred at rt for 1 h and filtered, the filtrate was concentrated at reduced pressure to leave the crude compound 6 (300 mg, crude) as oil. LC-MS (ESI) m/z: 325 [M+H]⁺.

### Step 4: Synthesis of 3-acrylamido-N-methyl-4-(3-(trifluoromethyl)benzyloxy)benzamide (Compound I-B-03)

To the mixture of 3-amino-N-methyl-4-(3-(trifluoromethyl)benzyloxy)benzamide (250 mg, 0.75 mmol) and Et₃ N (155 mg, 1.50 mmol) in DCM (15 mL) was added acryloyl chloride (70 mg, 0.75 mmol). The mixture was stirred at 0° C. for 1 h., and then purified directly by prep-HPLC to obtain the target compound 1-B-03 (51 mg, yield 17.5%) as solid. LC-MS (ESI) m/z: 379 [M+H]⁺. ¹H NMR (400 MHz, DMSO-d₆) δ (ppm) 9.60 (s, 1H), 8.46 -8.28 (m, 2H), 7.91 (s, 1H), 7.80 (d, J = 7.5 Hz, 1H), 7.69 (d, J = 8.0 Hz, 1H), 7.63 (dd, J = 14.8, 6.4 Hz, 2H), 7.18 (d, J = 8.6 Hz, 1H), 6.65 (dd, J = 16.7, 10.1 Hz, 1H), 6.27 (dd, J = 17.0, 1.5 Hz, 1H), 5.77 (dd, J = 10.4, 1.6 Hz, 1H), 5.38 (s, 2H), 2.75 (d, J = 4.4 Hz, 3H).

### I-a-03

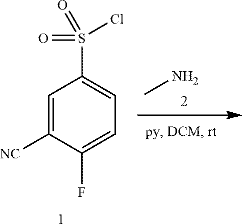

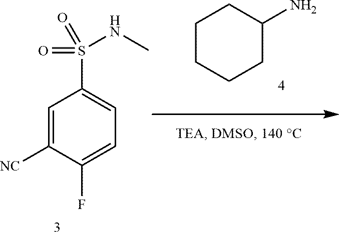

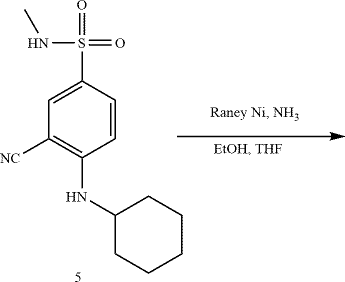

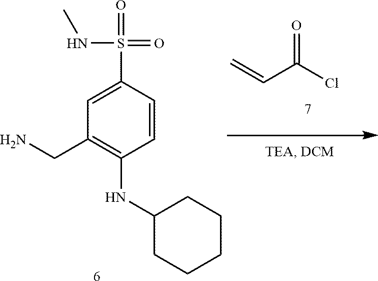

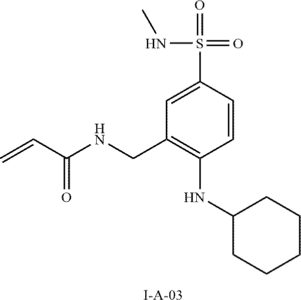

### Step 1: Synthesis of 3-cyano-4-fluoro-N-methylbenzenesulfonamide (Compound 3)

To the solution of 3-cyano-4-fluorobenzene-1-sulfonyl chloride (2 g, 9.1 mmol) in DCM (50 mL) was added methylamine solution in THF (2 M, 4.6 mL, 9.2 mmol) and pyridine (1.43 g, 18.2 mmol). The mixture was stirred at rt under N₂ for 1 h., concentrated and purified by flash column chromatography on silica gel (ethyl acetate in petroleum ether = 20% v/v) to obtain the target compound 3 (400 mg, yield 20.5%) as solid. LC-MS (ESI) m/z: 215 [M+H]⁺.

### Step 2: Synthesis of 3-cyano-4-(cyclohexylamino)-N-methylbenzenesulfonamide (Compound 5)

To the solution of 3-cyano-4-fluoro-N-methylbenzenesulfonamide (200 mg, 0.93 mmol) in DMSO (15 mL) was added cyclohexylamine (92 mg, 0.93 mmol) and Et₃ N (284 mg, 1.86 mmol). The mixture was stirred at 140° C. under N₂ for 2 h. After cooled down to rt the mixture was diluted with water (100 mL) and extracted with DCM (50 mL × 2), the combined organic was washed with brine (50 mL), dried over anhydrous Na₂SO₄, concentrated and purified by flash column chromatography on silica gel (ethyl acetate in petroleum ether = 20% v/v) to obtain the target compound 5 (200 mg, yield 73.2%) as solid. LC-MS (ESI) m/z: 294 [M+H]⁺.

### Step 3: Synthesis of 3-(aminomethyl)-4-(cyclohexylamino)-N-methylbenzenesulfonamide (Compound 6)

The mixture of 3-cyano-4-(cyclohexylamino)-N-methylbenzenesulfonamide (100 mg, 0.34 mmol), Raney nickel (14 mg, 0.04 mmol) and NH₃H₂O (concentrated, 8 mL) in EtOH (8 mL) and THF (8 mL) was stirred at rt under H₂ (1 atm) for 1 h. The mixture was filtered and concentrated at reduced pressure to leave the crude compound 6 (150 mg, crude) as oil. LC-MS (ESI) m/z: 298 [M+H]⁺.

### Step 4: Synthesis of N-(2-(cyclohexylamino)-5-(N-methylsulfamoyl)benzyl)acrylamide (Compound I-A-03)

To the mixture of 3-(aminomethyl)-4-(cyclohexylamino)-N-methylbenzenesulfonamide (100 mg, 0.34 mmol) and Et₃ N (68 mg, 0.68 mmol) in THF (15 mL) was added acryloyl chloride (30 mg, 0.34 mmol). The mixture was stirred at 0° C. for 1 h and concentrated in vacuum, the residue was purified by prep-HPLC to obtain the target compound I-A-03 (78 mg, yield 66.1%) as solid. LC-MS (ESI) m/z: 352.3 [M+H]⁺. ¹H NMR (500 MHz, DMSO-d₆) δ (ppm) 8.68 (t, J = 6.2 Hz, 1H), 7.46 (dd, J = 8.5, 2.5 Hz, 1H), 7.43 (d, J = 2.0 Hz, 1H), 7.00 (q, J = 5.0 Hz, 1H), 6.69 (d, J = 8.8 Hz, 1H), 6.24 (dd, J = 17.1, 9.5 Hz, 1H), 6.17 (dd, J = 17.1, 2.5 Hz, 1H), 5.90 (d, J = 7.4 Hz, 1H), 5.66 (dd, J = 9.7, 2.5 Hz, 1H), 4.29 (d, J = 6.2 Hz, 2H), 2.35 (d, J = 5.1 Hz, 3H), 1.92-1.82 (m, 2H), 1.76-1.66 (m, 2H), 1.63-1.54 (m, 1H), 1.40- 1.11(m, 5H).

### I-a-04

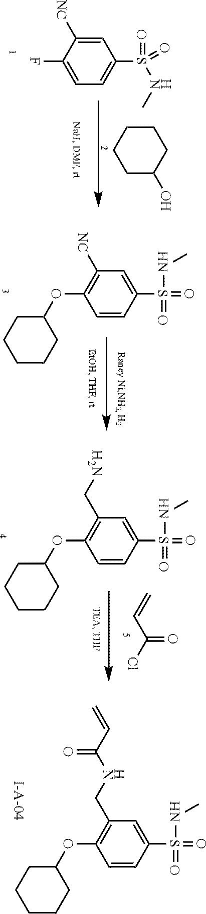

### Step 1: Synthesis of 3-cyano-4-(cyclohexyloxy)-N-methylbenzenesulfonamide (Compound 3)

To the solution of 3-cyano-4-fluoro-N-methylbenzenesulfonamide (93 mg, 0.93 mmol) in DMF (10 mL) was added NaH (60%, 44 mg, 1.12 mmol), the mixture was stirred at 0° C. under N₂0.5 h., and then cyclohexanol (200 mg, 0.93 mmol) was added. The resulting mixture was stirred at rt for 2 h., diluted with EtOAc (100 mL), washed with brine (50 mL), dried over anhydrous Na₂SO₄, concentrated and purified by flash column chromatography on silica gel (ethyl acetate in petroleum ether = 50% v/v) to obtain the target compound 3 (50 mg, yield 18.2%) as solid. LC-MS (ESI) m/z: 295 [M+H]⁺.

### Step 2: Synthesis of 3-(aminomethyl)-4-(cyclohexyloxy)-N-methylbenzenesulfonamide (Compound 4)

The mixture of 3-cyano-4-(cyclohexyloxy)-N-methylbenzenesulfonamide (40 mg, 0.14 mmol), Raney nickel (7 mg, 0.02 mmol) and concentrated NH₃H₂O (8 mL) in EtOH (8 mL) and THF (8 mL) was stirred at rt under H₂ (1 atm) for 1 h. The mixture was filtered through celite, the filtrate was concentrated at reduced pressure to leave the crude compound 4 (50 mg, crude) as oil. LC-MS (ESI) m/z: 299 [M+H]⁺.

### Step 3: Synthesis of N-(2-(cyclohexyloxy)-5-(N-methylsulfamoyl)benzyl)acrylamide (Compound I-A-04)

To the solution of 3-(aminomethyl)-4-(cyclohexyloxy)-N-methylbenzenesulfonamide (50 mg, 0.17 mmol) and Et₃N (34 mg, 0.34 mmol) in THF (10 mL) was added acryloyl chloride (15 mg, 0.17 mmol). The mixture was stirred at 0° C. under N₂ for 1 h., and then concentrated and purified by prep-HPLC to obtain the target compound I-A-04 (13 mg, yield 47.2%) as solid. LC-MS (ESI) m/z: 353.2 [M+H]+. ¹H NMR (400 MHz, DMSO-d₆) δ (ppm) 8.50 (t, J = 5.6 Hz, 1H), 7.62 (dd, J = 8.4, 1.6 Hz, 1H), 7.54 (d, J = 2.4, 1H), 7.30 (dd, J = 10.0, 4.8 Hz, 1H), 7.22 (d, J = 8.4, 1H), 6.33 (dd, J = 17.1, 10.2 Hz, 1H), 6.13 (dd, J = 17.1, 2.1 Hz, 1H), 5.64 (dd, J = 10.1, 2.2 Hz, 1H), 4.60-4.52 (m, 1H), 4.35 (d, J = 5.7 Hz, 2H), 2.36 (d, J = 5.0 Hz, 3H), 2.08 (s, 1H), 1.93-1.84 (m, 2H), 1.76-1.65 (m, 2H), 1.60 - 1.28 (m, 6H).

### I-a-05

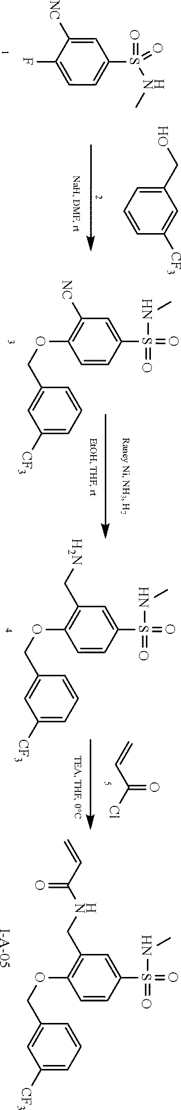

### Step 1: Synthesis of 3-cyano-N-methyl-4-(3-(trifluoromethyl)benzyloxy)benzenesulfonamide (Compound 3)

To the solution of (3-(trifluoromethyl)phenyl)methanol (250 mg, 1.16 mmol) in DMF (15 mL) was added NaH (56 mg, 1.40 mmol), the mixture was stirred at 0° C. under N₂ for 0.5 h., and then 3-cyano-4-fluoro-N-methylbenzenesulfonamide (204 mg, 1.16 mmol) was added. The resulting mixture was stirred at rt for 1 h., diluted with EtOAc (100 mL), washed with brine (50 mL), dried over anhydrous Na₂SO₄, concentrated and purified by flash column chromatography on silica gel (ethyl acetate in petroleum ether = 50% v/v) to obtain the target compound 3 (150 mg, yield 34.7%) as solid. LC-MS (ESI) m/z: 371 [M+H]⁺.

### Step 2: Synthesis of 3-(aminomethyl)-N-methyl-4-(3-(trifluoromethyl)benzyloxy)benzenesulfonamide (Compound 4)

The mixture of 3-cyano-N-methyl-4-(3-(trifluoromethyl)benzyloxy)benzenesulfonamide (100 mg, 0.28 mmol), Raney nickel (14 mg, 0.04 mmol) and concentrated NH₃H₂O (8 mL) in EtOH (8 mL) and THF (8 mL) was stirred at rt under H₂ (1 atm) for 1 h. The mixture was filtered through celite, the filtrate was concentrated at reduced pressure to leave the crude compound 4 (120 mg, crude) as oil. LC-MS (ESI) m/z: 375 [M+H]⁺.

### Step 3: Synthesis of N-(5-(N-methylsulfamoyl)-2-(3-(trifluoromethyl)benzyloxy)benzyl)acrylamide (Compound I-A-05)

To the solution of 3-(aminomethyl)-N-methyl-4-(3-(trifluoromethyl)benzyloxy)benzenesulfonamide (120 mg, 0.32 mmol) and Et₃ N (64 mg, 0.64 mmol) in THF (10 mL) was added acryloyl chloride (30 mg, 0.32 mmol). The mixture was stirred at 0° C. for 1 h., and then concentrated and purified by prep-HPLC to obtain the target compound I-A-05 (56 mg, yield 49.1%) as solid. LC-MS (ESI) m/z: 429 [M+H]⁺. ¹H NMR (400 MHz, DMSO-d₆) δ (ppm) 8.62 (t, J = 5.6 Hz, 1H), 7.87 (s, 1 H), 7.83 (d, J = 7.2 Hz, 1H), 7.73 (d, J = 7.6 Hz, 1H), 7.70 - 7.63 (m, 2H), 7.60 (d, J = 2.4 Hz, 1H), 7.35 (q, J = 4.8 Hz, 1H), 7.28 (d, J = 8.4 Hz, 1H), 6.33 (dd, J = 17.1, 10.2 Hz, 1H), 6.13 (dd, J = 17.1, 2.1 Hz, 1H), 5.64 (dd, J = 10.2, 2.1 Hz, 1H), 5.37 (s, 2H), 4.43 (d, J = 5.8 Hz, 2H), 2.37 (d, J = 5.0 Hz, 3H).

### I-b-04

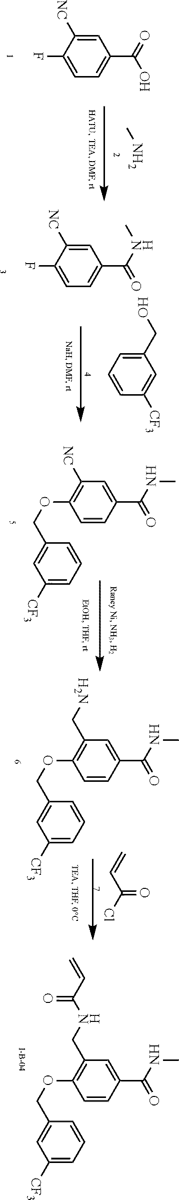

### Step 1: Synthesis of 3-cyano-4-fluoro-N-methylbenzamide (Compound 3)

To a solution of 3-cyano-4-fluorobenzoic acid (1000 mg, 6.0 mmol) in DMF (40 mL) was added methylamine solution in THF (2 M, 4.5 mL, 9.0 mmol), and then HATU (2760 mg, 7.3 mmol) and Et₃ N (1220 mg, 12 mmol) was added. The mixture was stirred at rt for 1 h., concentrated and purified by flash column chromatography on silica gel (ethyl acetate in petroleum ether =20% v/v) to obtain the target compound 3 (400 mg, yield 37.1%) as solid. LC-MS (ESI) m/z: 179 [M+H]⁺.

### Step 2: Synthesis of 3-cyano-N-methyl-4-(3-(trifluoromethyl)benzyloxy)benzamide (Compound 5)

To the solution of (3-(trifluoromethyl)phenyl)methanol (385 mg, 2.19 mmol) in DMF (15 mL) was added NaH (60%, 105 mg, 2.63 mmol), the mixture was stirred at 0° C. under N₂ for 0.5 h., and then 3-cyano-4-fluoro-N-methylbenzamide (390 mg, 2.19 mmol) was added. The resulting mixture was stirred at rt for 2 h., concentrated and purified by flash column chromatography on silica gel (ethyl acetate in petroleum ether = 20% v/v) to obtain the target compound 5 (200 mg, yield 27.3%) as solid. LC-MS (ESI) m/z: 335 [M+H]⁺.

### Step 3: Synthesis of 3-(aminomethyl)-N-methyl-4-(3-(trifluoromethyl)benzyloxy)benzamide (Compound 6)

The mixture of 3-cyano-N-methyl-4-(3-(trifluoromethyl)benzyloxy)benzamide (150 mg, 0.45 mmol), Raney nickel (18 mg, 0.05 mmol) and concentrated NH₃H₂O (8 mL) in EtOH (8 mL) and THF (8 mL) was stirred at rt under H₂ (1 atm) for 1 h. The mixture was filtered through celite, the filtrate was concentrated at reduced pressure to leave the crude compound 6 (200 mg, crude) as oil. LC-MS (ESI) m/z: 339 [M+H]⁺.

### Step 4: Synthesis of 3-(acrylamidomethyl)-N-methyl-4-(3-(trifluoromethyl)benzyloxy)benzamide (Compound I-B-04)

To the solution of 3-(aminomethyl)-N-methyl-4-(3-(trifluoromethyl)benzyloxy)benzamide (200 mg, 0.59 mmol) and Et₃N (119 mg, 1.18 mmol) in THF (10 mL) was added acryloyl chloride (53 mg, 0.59 mmol). The mixture was stirred at 0° C. under N₂ for 1 h., and then purified directly by prep-HPLC to obtain the target compound I-B-04 (20 mg, yield 8.6%) as solid. LC-MS (ESI) m/z: 393.1 [M+H]⁺. ¹H NMR (400 MHz, DMSO-d₆) δ (ppm) 8.50 (t, J = 4.8 Hz, 1H), 8.30 (d, J = 4.0 Hz, 1H), 7.91 - 7.60 (m, 6H), 7.14 (d, J = 8.1 Hz, 1H), 6.33 (dd, J = 17.0, 10.2 Hz, 1H), 6.13 (dd, J = 16.8, 1.6 Hz, 1H), 5.63 (dd, J = 10.1, 1.6 Hz, 1H), 5.34 (s, 2H), 4.41 (d, J = 5.5 Hz, 2H), 2.75 (d, J = 4.3 Hz, 3H).

### Ii-1

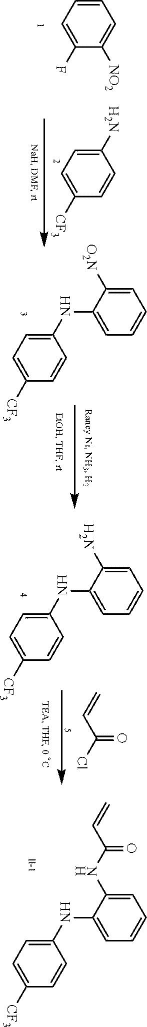

### Step 1: Synthesis of 2-nitro-N-(4-(trifluoromethyl)phenyl)aniline (Compound 3)

To the solution of 4-(trifluoromethyl)aniline (1.25 g, 7.8 mmol) in DMF (15 mL) was added NaH (60%, 340 mg, 14.2 mmol), the mixture was stirred at 0° C. under N₂ for 0.5 h., and then 1-fluoro-2-nitrobenzene (1 g, 7.1 mmol) was added. The resulting mixture was stirred at rt for 16 h., diluted with EtOAc (100 mL), washed with brine (50 mL), dried over anhydrous Na₂SO₄, concentrated and purified by flash column chromatography on silica gel (ethyl acetate in petroleum ether = 50% v/v) to obtain the target compound 3 (500 mg, yield 25.0%) as solid. LC-MS (ESI) m/z: 283 [M+H]⁺.

### Step 2: Synthesis of N1-(4-(trifluoromethyl)phenyl)benzene-1,2-diamine (Compound 4)

To the mixture of 2-nitro-N-(4-(trifluoromethyl)phenyl)aniline (450 mg, 1.6 mmol) and Raney nickel (65 mg, 0.16 mmol) in EtOH (10 mL) and THF (10 mL) was added N₂H₄•H₂O (200 mg, 4.0 mmol). The mixture was stirred at rt for 1 h and filtered, the filtrate was concentrated at reduced pressure to leave the crude compound 4 (400 mg, crude) as oil. LC-MS (ESI) m/z: 253 [M+H]⁺.

### Step 3: Synthesis of N-(2-(4-(trifluoromethyl)phenylamino)phenyl)acrylamide (Compound II-1)

To the solution of N1-(4-(trifluoromethyl)phenyl)benzene-1,2-diamine (350 mg, 1.39 mmol) and Et₃ N (281 mg, 2.78 mmol) in THF (20 mL) was added acryloyl chloride (125 mg, 1.39 mmol). The mixture was stirred at 0° C. under N₂ for 1 h and then concentrated in vacuum, the residue purified by prep-HPLC to obtain the target compound II-1 (188 mg, yield 44.2%) as solid. LC-MS (ESI) m/z: 307.1 [M+H]⁺. ¹H NMR (400 MHz, DMSO-d₆) δ (ppm) 9.61 (s, 1H), 8.00 (s, 1H), 7.72 (d, J = 7.4 Hz, 1H), 7.46 (d, J = 8.5 Hz, 2H), 7.33 (d, J = 7.6 Hz, 1H), 7.15 (dt, J = 15.1, 6.9 Hz, 2H), 6.90 (d, J = 8.5 Hz, 2H), 6.48 (dd, J = 17.0, 10.2 Hz, 1H), 6.23 (dd, J = 17.0, 1.5 Hz, 1H), 5.72 (d, J = 10.1 Hz, 1H).

### Ii-2

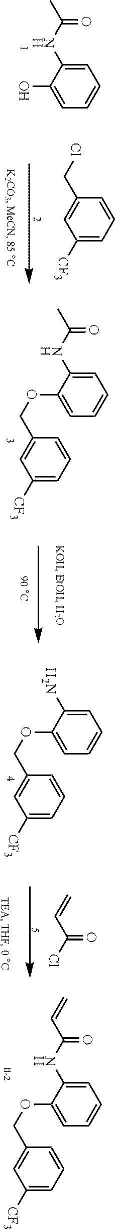

### Step 1: Synthesis of N-(2-(3-(trifluoromethyl)benzyloxy)phenyl)acetamide (Compound 3)

The mixture of 1-(chloromethyl)-3-(trifluoromethyl)benzene (1.41 g, 7.28 mmol), N-(2-hydroxyphenyl)acetamide (1 g, 6.62 mmol) and K₂CO₃ (2.74 g, 19.86 mmol) in MeCN (20 mL) was stirred at 85° C. under N₂ for 16 h. The mixture was filtered, the filtrate was concentrated and purified by flash column chromatography on silica gel (ethyl acetate in petroleum ether = 50% v/v) to obtain the target compound 3 (1.5 g, yield 73.5%) as solid. LC-MS (ESI) m/z: 310 [M+H]⁺.

### Step 2: Synthesis of 2-(3-(trifluoromethyl)benzyloxy)aniline (Compound 4)

The mixture of N-(2-(3-(trifluoromethyl)benzyloxy)phenyl)acetamide (500 mg, 4.62 mmol) and KOH (272 mg, 4.86 mmol) in EtOH (10 mL) and H₂O (10 mL) was stirred at 90° C. under N₂ for 16 h. The mixture was diluted with EtOAc (100 mL), washed with brine (50 mL), dried over anhydrous Na₂SO₄, filtered and concentrated to leave the crude compound 4 (400 mg, crude) as oil. LC-MS (ESI) m/z: 268 [M+H]⁺.

### Step 3: Synthesis of N-(2-(3-(trifluoromethyl)benzyloxy)phenyl)acrylamide (Compound II-2)

To the solution of 2-(3-(trifluoromethyl)benzyloxy)aniline (50 mg, 0.19 mmol) and Et₃ N (38 mg, 0.38 mmol) in THF (10 mL) was added acryloyl chloride (17 mg, 0.19 mmol). The mixture was stirred at 0° C. under N₂ for 1 h., and then concentrated and purified by prep-HPLC to obtain the target compound 11-2 (95 mg, yield 22.6%) as solid. LC-MS (ESI) m/z: 322.1 [M+H]⁺. ¹H NMR (400 MHz, DMSO-d₆) δ (ppm) 9.47 (s, 1H), 7.90 (s, 2H), 7.80 (d, J = 7.5 Hz, 1H), 7.65 (dt, J = 15.3, 7.7 Hz, 2H), 7.21 - 7.07 (m, 2H), 6.98 - 6.90 (m, 1H), 6.66 (dd, J = 17.0, 10.2 Hz, 1H), 6.25 (dd, J = 17.0, 1.9 Hz, 1H), 5.74 (dd, J = 10.2, 1.9 Hz, 1H), 5.32 (s, 2H).

### Synthesis of Exemplary Compounds

### I-a-01

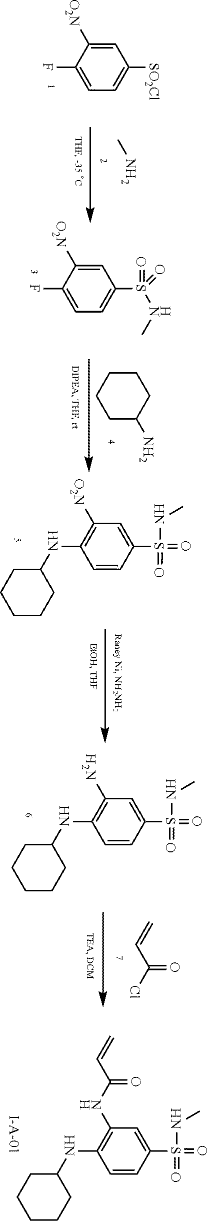

### Step 1: Synthesis of 4-fluoro-N-methyl-3-nitrobenzenesulfonamide (Compound 3)

To the mixture of 4-fluoro-3-nitrobenzene-1-sulfonyl chloride (2 g, 8.4 mmol) in THF (80 mL) was added Et₃ N (5.08 g, 50.4 mmol), the mixture was stirred at -35° C. under N₂ for 10 min., and then the solution of methylamine in THF (1 M, 10 mL, 10 mmol) was added dropwise, the mixture was stirred at -35° C. for 1 hour, diluted with EtOAc (100 mL), washed with brine (50 mL), dried over anhydrous Na₂SO₄, filtered and concentrated to leave the crude product (2 g) as a yellow oil, which was purified by flash column chromatography on silica gel (ethyl acetate in petroleum ether = 50% v/v) to obtain the target compound 3 (1.33 g, yield 68.2%) as solid. ¹H NMR (400 MHz, DMSO-d₆) δ (ppm) 8.47 (dd, J = 7.0, 2.3 Hz, 1H), 8.18 (ddd, J = 8.7, 4.0, 2.4 Hz, 1H), 7.93 - 7.80 (m, 2H), 2.48 (d, J = 4.9 Hz, 3H).

### Step 2: Synthesis of 4-(cyclohexylamino)-N-methyl-3-nitrobenzenesulfonamide (Compound 5)

To the mixture of 4-fluoro-N-methyl-3-nitrobenzenesulfonamide (400 mg, 1.71 mmol) in THF (40 mL) was added cyclohexylamine (168.7 mg, 1.71 mmol) and DIPEA (655.5 mg, 5.12 mmol). The mixture was stirred at rt under N₂ for 3 h., diluted with EtOAc (100 mL), washed with brine (50 mL), dried over anhydrous Na₂SO₄, concentrated and purified by flash column chromatography on silica gel (ethyl acetate in petroleum ether = 20% v/v) to afford the target compound 5 (510 mg, yield 95.5%) as pale yellow solid. LC-MS (ESI) m/z: 314 [M+H]⁺.

### Step 3: Synthesis of 3-amino-4-(cyclohexylamino)-N-methylbenzenesulfonamide (Compound 6)

To the mixture of 4-(cyclohexylamino)-N-methyl-3-nitrobenzenesulfonamide (250 mg, 0.8 mmol) and Raney nickel (40 mg, 0.1 mmol) in EtOH (10 mL) and THF (10 mL) was added N₂H₄•H₂O (100 mg, 2.0 mmol). The mixture was stirred at rt for 1 h and filtered, the filtrate was concentrated and purified by flash column chromatography on silica gel (ethyl acetate in petroleum ether = 40% v/v) to obtain the target compound 6 (200 mg, yield 88.5%) as solid. LC-MS (ESI) m/z: 284 [M+H]⁺.

### Step 4: Synthesis of N-(2-(cyclohexylamino)-5-(N-methylsulfamoyl)phenyl)acrylamide (Compound I-A-01)

To the mixture of 3-amino-4-(cyclohexylamino)-N-methylbenzenesulfonamide (150 mg, 0.51 mmol) and Et₃ N (102 mg, 1.02 mmol) in DCM (15 mL) was added acryloyl chloride (48 mg, 0.51 mmol). The mixture was stirred at 0° C. under N₂ for 1 h, and concentrated under vacuum, the residue was purified by prep-HPLC (MeCN/H₂O/TFA) to obtain the target compound I-A-01 (92 mg, yield 51.6%) as solid. LC-MS (ESI) m/z: 338.2 [M+H]⁺. ¹H NMR (400 MHz, DMSO-d₆) δ (ppm) 9.47 (s, 1H), 7.71 (s, 1H), 7.40 (d, J = 8.7 Hz, 1H), 7.07 (d, J = 5.0 Hz, 1H), 6.81 (d, J = 8.8 Hz, 1H), 6.51 (dd, J = 17.0, 10.2 Hz, 1H), 6.26 (d, J = 16.9 Hz, 1H), 5.78 (d, J = 9.9 Hz, 1H), 5.40 (d, J = 7.5 Hz, 1H), 2.36 (d, J = 5.0 Hz, 3H), 1.95 (d, J = 10.8 Hz, 2H), 1.72 (d, J = 10.0 Hz, 2H), 1.62 (d, J = 11.6 Hz, 1H), 1.44-1.10 (m, 5H).

### I-a-02

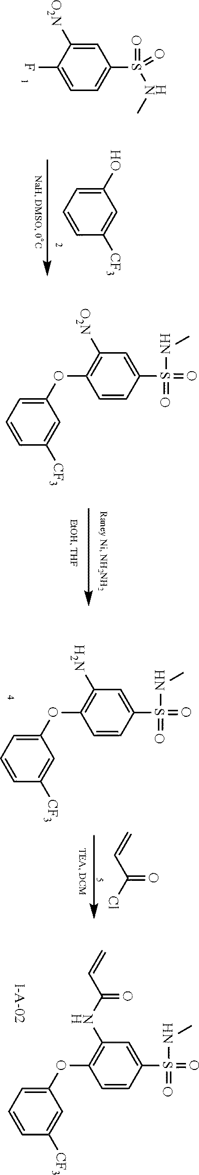

### Step 1: Synthesis of N-methyl-3-nitro-4-(3-(trifluoromethyl)phenoxy)benzenesulfonamide (Compound 3)

To the solution of 4-fluoro-N-methyl-3-nitrobenzenesulfonamide (300 mg, 1.28 mmol) in DMSO (15 mL) was added NaH (60%, 61.5 mg, 1.53 mmol), the mixture was stirred at 0° C. under N₂ for 0.5 h., and then 3-(trifluoromethyl)phenol (207 mg, 1.29 mmol) was added. The mixture was stirred at rt overnight, diluted with EtOAc (100 mL), washed with brine (50 mL), dried over anhydrous Na₂SO₄, concentrated and purified by flash column chromatography on silica gel (ethyl acetate in petroleum ether = 50% v/v) to obtain the target compound 3 (200 mg, yield 41.4%) as yellow oil. LC-MS (ESI) m/z: 377 [M+H]⁺.

### Step 2: Synthesis of 3-amino-N-methyl-4-(3-(trifluoromethyl)phenoxy)benzenesulfonamide (Compound 4)

To the mixture of N-methyl-3-nitro-4-(3-(trifluoromethyl)phenoxy) benzenesulfonamide (150 mg, 0.4 mmol) and Raney nickel (16 mg, 0.04 mmol) in EtOH (10 mL) and THF (10 mL) was added N₂H₄•H₂O (20 mg, 0.4 mmol). The mixture was stirred at rt for 1 h and filtered, the filtrate was concentrated at reduced pressure to leave the crude compound 4 (130 mg, crude) as oil. LC-MS (ESI) m/z: 347 [M+H]⁺.

### Step 3: Synthesis of N-(5-(N-methylsulfamoyl)-2-(3-(trifluoromethyl)phenoxy)phenyl)acrylamide (Compound I-A-02)

To the mixture of 3-amino-N-methyl-4-(3-(trifluoromethyl)phenoxy)benzenesulfonamide (100 mg, 0.29 mmol) and Et₃ N (58 mg, 0.58 mmol) in DCM (15 mL) was added acryloyl chloride (26 mg, 0.29 mmol). The mixture was stirred at 0° C. under N₂ for 1 h, and then concentrated under vacuum, the residue was purified by prep-HPLC (MeCN/H₂O/TFA) to obtain the target compound I-A-02 (11 mg, yield 9.6%) as solid. LC-MS (ESI) m/z: 401 [M+H]⁺. ¹H NMR (400 MHz, CD₃OD) δ (ppm) 8.58 (d, J = 2.1 Hz, 1H), 7.61 - 7.48 (m, 2H), 7.43 (d, J = 7.6 Hz, 1H), 7.34 (s, 1H), 7.25 (d, J = 8.1 Hz, 1H), 6.94 (d, J = 8.6 Hz, 1H), 6.46 (dd, J = 17.0, 10.2 Hz, 1H), 6.29 (dd, J = 17.0, 1.7 Hz, 1H), 5.69 (dd, J = 10.2, 1.7 Hz, 1H), 4.53 (s, 1H), 2.48 (s, 3H).

### I-b-01

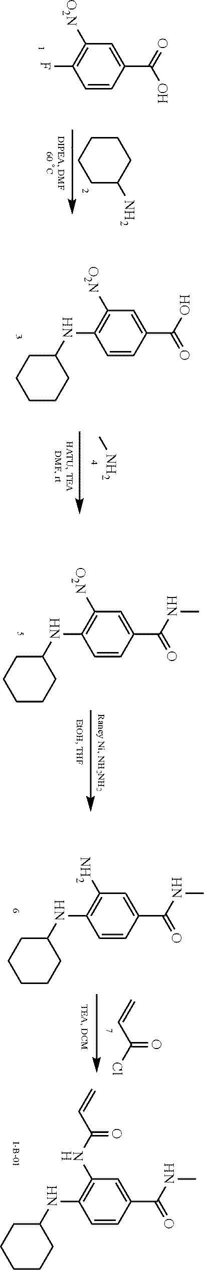

### Step 1: Synthesis of 4-(cyclohexylamino)-3-nitrobenzoic Acid (Compound 3)

The mixture of 4-fluoro-3-nitrobenzoic acid (1 g, 5.4 mmol), cyclohexanamine (534 mg, 5.4 mmol) and DIPEA (2.1 g, 16.2 mmol) in DMF (25 mL) re was stirred at 60° C. under N₂ for 3 h. The resulting suspension was filtered, the cake was dried under vacuum to obtain the target compound 3 (1 g, crude) as solid. LC-MS (ESI) m/z: 265 [M+H]⁺.

### Step 2: Synthesis of 4-(cyclohexylamino)-N-methyl-3-nitrobenzamide (Compound 5)

To the solution of 4-(cyclohexylamino)-3-nitrobenzoic acid (1.5 g, 4.4 mmol) in DMF (20 mL) was added methylamine solution in THF (2.1 mL, 2 mol/L, 4.4 mmol), and then HATU (2 g, 5.28 mmol) and Et₃ N (885 mg, 8.8 mmol) was added. The mixture was stirred at rt for 3 h., diluted with EtOAc (100 mL), washed with brine (50 mL), dried over anhydrous Na₂SO₄, filtered, concentrated and purified by flash column chromatography on silica gel (ethyl acetate in petroleum ether = 20% v/v) to obtain the target compound 5 (300 mg, yield 19.1%) as solid. LC-MS (ESI) m/z: 278 [M+H]⁺.

### Step 3: Synthesis of 3-amino-4-(cyclohexylamino)-N-methylbenzamide (Compound 6)

To the mixture of 4-(cyclohexylamino)-N-methyl-3-nitrobenzamide (240 mg, 0.88 mmol) and Raney nickel (32 mg, 0.08 mmol) in EtOH (10 mL) and THF (10 mL) was added N₂H₄•H₂O (200 mg, 4.0 mmol). The mixture was stirred at rt for 1 h and filtered, the filtrate was concentrated at reduced pressure to leave the crude compound 6 (100 mg, crude) as oil. LC-MS (ESI) m/z: 248 [M+H]⁺.

### Step 4: Synthesis of 3-acrylamido-4-(cyclohexylamino)-N-methylbenzamide (Compound I-B-01)

To the mixture of 3-amino-4-(cyclohexylamino)-N-methylbenzamide (100 mg, 0.4 mmol) and Et₃ N (80 mg, 0.8 mmol) in DCM (10 mL) was added acryloyl chloride (36 mg, 0.4 mmol). The mixture was stirred at 0° C. for 15 minutes, and then purified directly by prep-HPLC (MeCN/H₂O/TFA) to obtain the target compound I-B-01 (38 mg, yield 31.4%) as solid. LC-MS (ESI) m/z: 302.3 [M+H]⁺. ¹H NMR (400 MHz, DMSO-d₆) δ (ppm) 9.45 (s, 1H), 8.07 (d, J = 4.4 Hz, 1H), 7.73 (d, J = 1.5 Hz, 1H), 7.56 (d, J = 8.5 Hz, 1H), 6.69 (d, J = 8.7 Hz, 1H), 6.50 (dd, J = 17.0, 10.2 Hz, 1H), 6.23 (dd, J = 17.0, 1.8 Hz, 1H), 5.76 (dd, J = 10.2, 1.6 Hz, 1H), 5.08 (d, J = 7.6 Hz, 1H), 3.34 - 3.29 (m, 1H), 2.72 (d, J = 4.4 Hz, 3H), 1.93 (d, J = 10.2 Hz, 2H), 1.79 - 1.51 (m, 3H), 1.43-1.11 (m, 5H).

### I-b-02

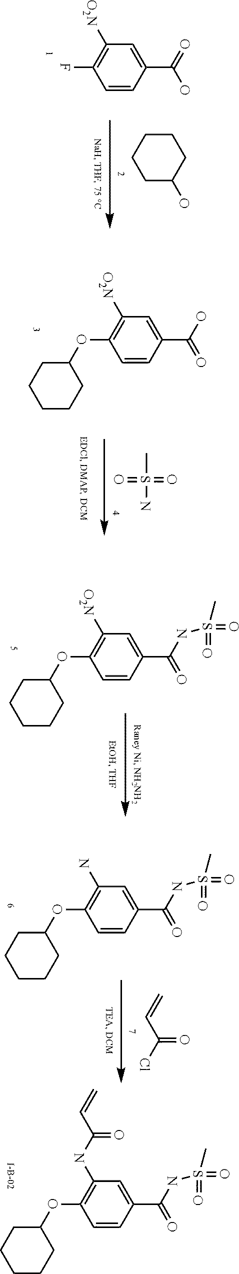

### Step 1: Synthesis of 4-(cyclohexyloxy)-3-nitrobenzoic Acid (Compound 3)

To the solution of cyclohexanol (1.08 g, 10.8 mmol) in THF (50 mL) was added NaH (60%, 520 mg, 13.0 mmol), the mixture was stirred at 0° C. under N₂ for 0.5 h., and then 4-fluoro-3-nitrobenzoic acid (2 g, 10.8 mmol) was added. The mixture was stirred at 75° C. for 4 h. After cooled down to rt the mixture was diluted with EtOAc (100 mL), washed with brine (50 mL), dried over anhydrous Na₂SO₄, filtered and concentrated at reduced pressure to leave the crude compound 3 (2 g, crude) as oil. LC-MS (ESI) m/z: no MS.

### Step 2: Synthesis of 4-(cyclohexyloxy)-N-(methylsulfonyl)-3-nitrobenzamide (Compound 5)

To the solution of 4-(cyclohexyloxy)-3-nitrobenzoic acid (2 g, 7.55 mmol) in DCM (100 mL) was added EDCI (2.15 g, 11.32 mmol) and DMAP (2.11 g, 17.35 mmol). The mixture was stirred at rt for 10 min., and then methanesulfonamide (1.06 g, 11.32 mmol) was added. The mixture was stirred at rt overnight, concentrated and purified by flash column chromatography on silica gel (methylene chloride in methanol = 20% v/v) to obtain the target compound 5 (1.22 g, yield 47.2%) as solid. LC-MS (ESI) m/z: 343 [M+H]⁺.

### Step 3: Synthesis of 3-amino-4-(cyclohexyloxy)-N-(methylsulfonyl)benzamide (Compound 6)

To the mixture of 4-(cyclohexyloxy)-N-(methylsulfonyl)-3-nitrobenzamide (500 mg, 1.4 mmol) and Raney nickel (60 mg, 0.2 mmol) in EtOH (25 mL) and THF (25 mL) was added N₂H₄•H₂O (200 mg, 4.0 mmol). The mixture was stirred at rt for 1 h and filtered, the filtrate was concentrated at reduced pressure to leave the crude compound 6 (200 mg, crude) as oil. LC-MS (ESI) m/z: 313 [M+H]⁺.

### Step 4: Synthesis of 3-acrylamido-4-(cyclohexyloxy)-N-(methylsulfonyl)benzamide (Compound I-B-02)

To the mixture of 3-amino-4-(cyclohexyloxy)-N-(methylsulfonyl)benzamide (150 mg, 0.45 mmol) and Et₃ N (90 mg, 0.90 mmol) in DCM (15 mL) was added acryloyl chloride (39 mg, 0.45 mmol). The mixture was stirred at 0° C. for 1 h., and then purified by prep-HPLC to obtain the target compound I-B-02 (25 mg, yield 14.2%) as solid. LC-MS (ESI) m/z: 367 [M+H]⁺. ¹H NMR (400 MHz, DMSO-d₆) δ (ppm) 9.15 (s, 1H), 8.47 (s, 1H), 8.15 (s, 1H), 7.69 (d, J = 8.3 Hz, 1H), 7.12 (dd, J = 45.1, 8.6 Hz, 1H), 6.65 (dd, J = 16.5, 10.4 Hz, 1H), 6.24 (d, J = 17.1 Hz, 1H), 5.73 (d, J = 10.0 Hz, 1H), 4.44 (br, 1H), 2.94 (d, J = 26.6 Hz, 3H), 1.98-1.94 (m, 2H), 1.81-1.65 (m, 2H), 1.61-1.44 (m, 3H), 1.43 - 1.18 (m, 3H).

### I-b-03

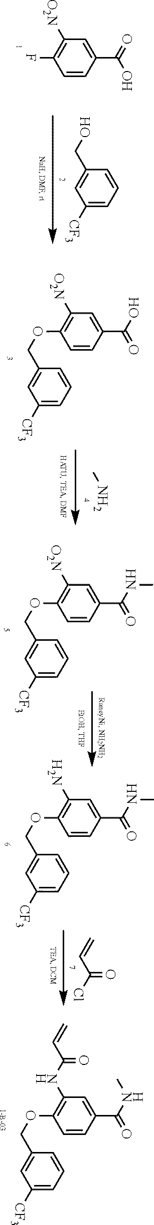

### Step 1: Synthesis of 3-nitro-4-(3-(trifluoromethyl)benzyloxy)benzoic Acid (Compound 3)

To the solution of 4-fluoro-3-nitrobenzoic acid (1 g, 5.4 mmol) in DMF (20 mL) was added NaH (60%, 260 mg, 6.5 mmol), the mixture was stirred at 0° C. under N₂ atmosphere for 0.5 h., and then (3-(trifluoromethyl)phenyl)methanol (1.52 mg, 8.6 mmol) was added. The mixture was stirred at rt for 2 h., diluted with EtOAc (100 mL), washed with brine (50 mL), dried over anhydrous Na₂SO₄, filtered and concentrated at reduced pressure to leave the target compound 3 (600 mg, yield 32.6%) as solid. LC-MS (ESI) m/z: 365 [M+Na]⁺.

### Step 2: Synthesis of N-methyl-3-nitro-4-(3-(trifluoromethyl)benzyloxy)benzamide (Compound 5)

To the solution of 3-nitro-4-(3-(trifluoromethyl)benzyloxy)benzoic acid (500 mg, 1.45 mmol) in DMF (15 mL) was added methylamine solution in THF (2 M,0.7 mL, 1.4 mmol), and then HATU (668.5 mg, 1.75 mmol) and Et₃ N (295 mg, 2.9 mmol) was added. The mixture was stirred at rt for 1 h., concentrated and purified by flash column chromatography on silica gel (ethyl acetate in petroleum ether = 20% v/v) to obtain the target compound 5 (500 mg, yield 96.3%) as solid. LC-MS (ESI) m/z: 355 [M+H]⁺.

### Step 3: Synthesis of 3-amino-N-methyl-4-(3-(trifluoromethyl)benzyloxy)benzamide (Compound 6)

To the mixture of N-methyl-3-nitro-4-(3-(trifluoromethyl)benzyloxy)benzamide (500 mg, 1.4 mmol) and Raney nickel (60 mg, 0.2 mmol) in EtOH (15 mL) and THF (15 mL) was added N₂H₄•H₂O (200 mg, 4.0 mmol). The mixture was stirred at rt for 1 h and filtered, the filtrate was concentrated at reduced pressure to leave the crude compound 6 (300 mg, crude) as oil. LC-MS (ESI) m/z: 325 [M+H]⁺.

### Step 4: Synthesis of 3-acrylamido-N-methyl-4-(3-(trifluoromethyl)benzyloxy)benzamide (Compound I-B-03)

To the mixture of 3-amino-N-methyl-4-(3-(trifluoromethyl)benzyloxy)benzamide (250 mg, 0.75 mmol) and Et₃N (155 mg, 1.50 mmol) in DCM (15 mL) was added acryloyl chloride (70 mg, 0.75 mmol). The mixture was stirred at 0° C. for 1 h., and then purified directly by prep-HPLC to obtain the target compound I-B-03 (51 mg, yield 17.5%) as solid. LC-MS (ESI) m/z: 379 [M+H]⁺. ¹H NMR (400 MHz, DMSO-d₆) δ (ppm) 9.60 (s, 1H), 8.46 -8.28 (m, 2H), 7.91 (s, 1H), 7.80 (d, J = 7.5 Hz, 1H), 7.69 (d, J = 8.0 Hz, 1H), 7.63 (dd, J = 14.8, 6.4 Hz, 2H), 7.18 (d, J = 8.6 Hz, 1H), 6.65 (dd, J = 16.7, 10.1 Hz, 1H), 6.27 (dd, J = 17.0, 1.5 Hz, 1H), 5.77 (dd, J = 10.4, 1.6 Hz, 1H), 5.38 (s, 2H), 2.75 (d, J = 4.4 Hz, 3H).

### I-a-03

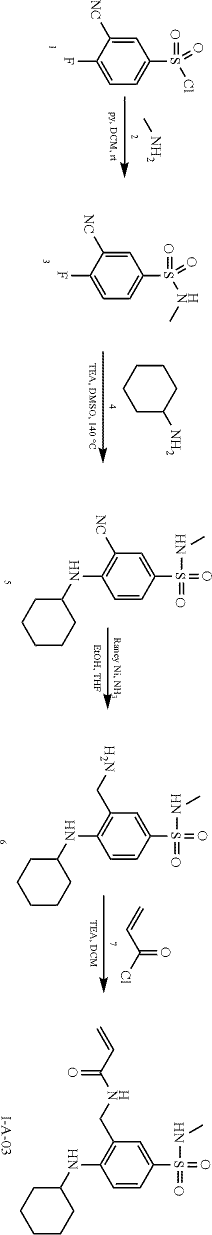

### Step 1: Synthesis of 3-cyano-4-fluoro-N-methylbenzenesulfonamide (Compound 3)

To the solution of 3-cyano-4-fluorobenzene-1-sulfonyl chloride (2 g, 9.1 mmol) in DCM (50 mL) was added methylamine solution in THF (2 M, 4.6 mL, 9.2 mmol) and pyridine (1.43 g, 18.2 mmol). The mixture was stirred at rt under N₂ for 1 h., concentrated and purified by flash column chromatography on silica gel (ethyl acetate in petroleum ether = 20% v/v) to obtain the target compound 3 (400 mg, yield 20.5%) as solid. LC-MS (ESI) m/z: 215 [M+H]⁺.

### Step 2: Synthesis of 3-cyano-4-(cyclohexylamino)-N-methylbenzenesulfonamide (Compound 5)

To the solution of 3-cyano-4-fluoro-N-methylbenzenesulfonamide (200 mg, 0.93 mmol) in DMSO (15 mL) was added cyclohexylamine (92 mg, 0.93 mmol) and Et₃ N (284 mg, 1.86 mmol). The mixture was stirred at 140° C. under N₂ for 2 h. After cooled down to rt the mixture was diluted with water (100 mL) and extracted with DCM (50 mL × 2), the combined organic was washed with brine (50 mL), dried over anhydrous Na₂SO₄, concentrated and purified by flash column chromatography on silica gel (ethyl acetate in petroleum ether = 20% v/v) to obtain the target compound 5 (200 mg, yield 73.2%) as solid. LC-MS (ESI) m/z: 294 [M+H]⁺.

### Step 3: Synthesis of 3-(aminomethyl)-4-(cyclohexylamino)-N-methylbenzenesulfonamide (Compound 6)

The mixture of 3-cyano-4-(cyclohexylamino)-N-methylbenzenesulfonamide (100 mg, 0.34 mmol), Raney nickel (14 mg, 0.04 mmol) and NH₃H₂O (concentrated, 8 mL) in EtOH (8 mL) and THF (8 mL) was stirred at rt under H₂ (1 atm) for 1 h. The mixture was filtered and concentrated at reduced pressure to leave the crude compound 6 (150 mg, crude) as oil. LC-MS (ESI) m/z: 298 [M+H]⁺.

### Step 4: Synthesis of N-(2-(cyclohexylamino)-5-(N-methylsulfamoyl)benzyl)acrylamide (Compound 1-A-03)

To the mixture of 3-(aminomethyl)-4-(cyclohexylamino)-N-methylbenzenesulfonamide (100 mg, 0.34 mmol) and Et₃ N (68 mg, 0.68 mmol) in THF (15 mL) was added acryloyl chloride (30 mg, 0.34 mmol). The mixture was stirred at 0° C. for 1 h and concentrated in vacuum, the residue was purified by prep-HPLC to obtain the target compound I-A-03 (78 mg, yield 66.1%) as solid. LC-MS (ESI) m/z: 352.3 [M+H]⁺. ¹H NMR (500 MHz, DMSO-d₆) δ (ppm) 8.68 (t, J = 6.2 Hz, 1H), 7.46 (dd, J = 8.5, 2.5 Hz, 1H), 7.43 (d, J = 2.0 Hz, 1H), 7.00 (q, J = 5.0 Hz, 1H), 6.69 (d, J = 8.8 Hz, 1H), 6.24 (dd, J = 17.1, 9.5 Hz, 1H), 6.17 (dd, J = 17.1, 2.5 Hz, 1H), 5.90 (d, J = 7.4 Hz, 1H), 5.66 (dd, J = 9.7, 2.5 Hz, 1H), 4.29 (d, J = 6.2 Hz, 2H), 2.35 (d, J = 5.1 Hz, 3H), 1.92-1.82 (m, 2H), 1.76-1.66 (m, 2H), 1.63-1.54 (m, 1H), 1.40- 1.1 1(m, 5H).

### I-a-04

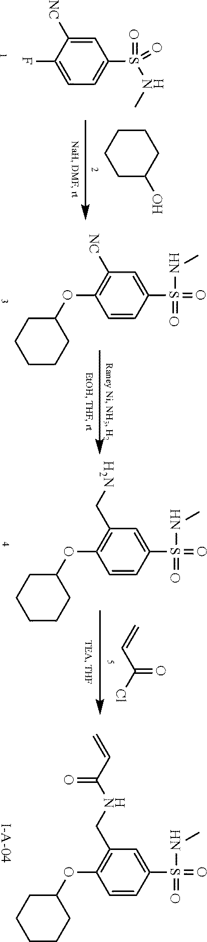

### Step 1: Synthesis of 3-cyano-4-(cyclohexyloxy)-N-methylbenzenesulfonamide (Compound 3)

To the solution of 3-cyano-4-fluoro-N-methylbenzenesulfonamide (93 mg, 0.93 mmol) in DMF (10 mL) was added NaH (60%, 44 mg, 1.12 mmol), the mixture was stirred at 0° C. under N₂0.5 h., and then cyclohexanol (200 mg, 0.93 mmol) was added. The resulting mixture was stirred at rt for 2 h., diluted with EtOAc (100 mL), washed with brine (50 mL), dried over anhydrous Na₂SO₄, concentrated and purified by flash column chromatography on silica gel (ethyl acetate in petroleum ether = 50% v/v) to obtain the target compound 3 (50 mg, yield 18.2%) as solid. LC-MS (ESI) m/z: 295 [M+H]⁺.

### Step 2: Synthesis of 3-(aminomethyl)-4-(cyclohexyloxy)-N-methylbenzenesulfonamide (Compound 4)

The mixture of 3-cyano-4-(cyclohexyloxy)-N-methylbenzenesulfonamide (40 mg, 0.14 mmol), Raney nickel (7 mg, 0.02 mmol) and concentrated NH₃H₂O (8 mL) in EtOH (8 mL) and THF (8 mL) was stirred at rt under H₂ (1 atm) for 1 h. The mixture was filtered through celite, the filtrate was concentrated at reduced pressure to leave the crude compound 4 (50 mg, crude) as oil. LC-MS (ESI) m/z: 299 [M+H]⁺.

### Step 3: Synthesis of N-(2-(cyclohexyloxy)-5-(N-methylsulfamoyl)benzyl)acrylamide (Compound I-A-04)

To the solution of 3-(aminomethyl)-4-(cyclohexyloxy)-N-methylbenzenesulfonamide (50 mg, 0.17 mmol) and Et₃ N (34 mg, 0.34 mmol) in THF (10 mL) was added acryloyl chloride (15 mg, 0.17 mmol). The mixture was stirred at 0° C. under N₂ for 1 h., and then concentrated and purified by prep-HPLC to obtain the target compound I-A-04 (13 mg, yield 47.2%) as solid. LC-MS (ESI) m/z: 353.2 [M+H]⁺. ¹H NMR (400 MHz, DMSO-d₆) δ (ppm) 8.50 (t, J = 5.6 Hz, 1H), 7.62 (dd, J = 8.4, 1.6 Hz, 1H), 7.54 (d, J = 2.4, 1H), 7.30 (dd, J = 10.0, 4.8 Hz, 1H), 7.22 (d, J = 8.4, 1H), 6.33 (dd, J = 17.1, 10.2 Hz, 1H), 6.13 (dd, J = 17.1, 2.1 Hz, 1H), 5.64 (dd, J = 10.1, 2.2 Hz, 1H), 4.60-4.52 (m, 1H), 4.35 (d, J = 5.7 Hz, 2H), 2.36 (d, J = 5.0 Hz, 3H), 2.08 (s, 1H), 1.93-1.84 (m, 2H), 1.76-1.65 (m, 2H), 1.60 - 1.28 (m, 6H).

### I-a-05

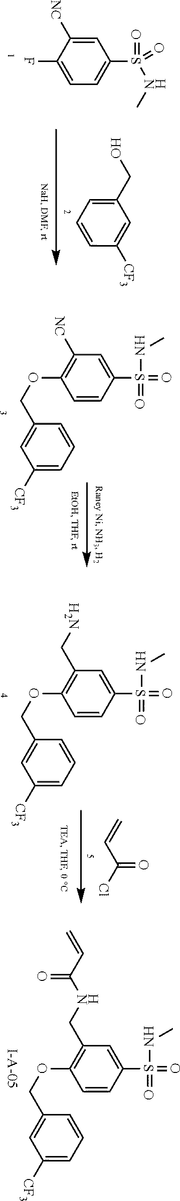

### Step 1: Synthesis of 3-cyano-N-methyl-4-(3-(trifluoromethyl)benzyloxy)benzenesulfonamide (Compound 3)

To the solution of (3-(trifluoromethyl)phenyl)methanol (250 mg, 1.16 mmol) in DMF (15 mL) was added NaH (56 mg, 1.40 mmol), the mixture was stirred at 0° C. under N₂ for 0.5 h., and then 3-cyano-4-fluoro-N-methylbenzenesulfonamide (204 mg, 1.16 mmol) was added. The resulting mixture was stirred at rt for 1 h., diluted with EtOAc (100 mL), washed with brine (50 mL), dried over anhydrous Na₂SO₄, concentrated and purified by flash column chromatography on silica gel (ethyl acetate in petroleum ether = 50% v/v) to obtain the target compound 3 (150 mg, yield 34.7%) as solid. LC-MS (ESI) m/z: 371 [M+H]⁺.

### Step 2: Synthesis of 3-(aminomethyl)-N-methyl-4-(3-(trifluoromethyl)benzyloxy)benzenesulfonamide (Compound 4)

The mixture of 3-cyano-N-methyl-4-(3-(trifluoromethyl)benzyloxy)benzenesulfonamide (100 mg, 0.28 mmol), Raney nickel (14 mg, 0.04 mmol) and concentrated NH₃H₂O (8 mL) in EtOH (8 mL) and THF (8 mL) was stirred at rt under H₂ (1 atm) for 1 h. The mixture was filtered through celite, the filtrate was concentrated at reduced pressure to leave the crude compound 4 (120 mg, crude) as oil. LC-MS (ESI) m/z: 375 [M+H]⁺.

### Step 3: Synthesis of N-(5-(N-methylsulfamoyl)-2-(3-(trifluoromethyl)benzyloxy)benzyl)acrylamide (Compound I-A-05)

To the solution of 3-(aminomethyl)-N-methyl-4-(3-(trifluoromethyl)benzyloxy)benzenesulfonamide (120 mg, 0.32 mmol) and Et₃ N (64 mg, 0.64 mmol) in THF (10 mL) was added acryloyl chloride (30 mg, 0.32 mmol). The mixture was stirred at 0° C. for 1 h., and then concentrated and purified by prep-HPLC to obtain the target compound I-A-05 (56 mg, yield 49.1%) as solid. LC-MS (ESI) m/z: 429 [M+H]⁺. ¹H NMR (400 MHz, DMSO-d₆) δ (ppm) 8.62 (t, J = 5.6 Hz, 1H), 7.87 (s, 1 H), 7.83 (d, J = 7.2 Hz, 1H), 7.73 (d, J = 7.6 Hz, 1H), 7.70 - 7.63 (m, 2H), 7.60 (d, J = 2.4 Hz, 1H), 7.35 (q, J = 4.8 Hz, 1H), 7.28 (d, J = 8.4 Hz, 1H), 6.33 (dd, J = 17.1, 10.2 Hz, 1H), 6.13 (dd, J = 17.1, 2.1 Hz, 1H), 5.64 (dd, J = 10.2, 2.1 Hz, 1H), 5.37 (s, 2H), 4.43 (d, J = 5.8 Hz, 2H), 2.37 (d, J = 5.0 Hz, 3H).

### I-b-04

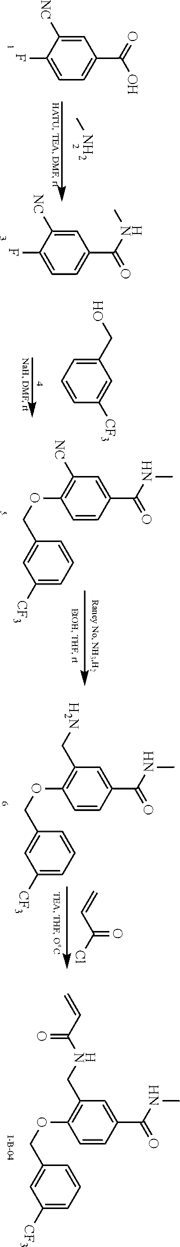

### Step 1: Synthesis of 3-cyano-4-fluoro-N-methylbenzamide (Compound 3)

To a solution of 3-cyano-4-fluorobenzoic acid (1000 mg, 6.0 mmol) in DMF (40 mL) was added methylamine solution in THF (2 M, 4.5 mL, 9.0 mmol), and then HATU (2760 mg, 7.3 mmol) and Et₃ N (1220 mg, 12 mmol) was added. The mixture was stirred at rt for 1 h., concentrated and purified by flash column chromatography on silica gel (ethyl acetate in petroleum ether =20% v/v) to obtain the target compound 3 (400 mg, yield 37.1%) as solid. LC-MS (ESI) m/z: 179 [M+H]⁺.

### Step 2: Synthesis of 3-cyano-N-methyl-4-(3-(trifluoromethyl)benzyloxy)benzamide (Compound 5)

To the solution of (3-(trifluoromethyl)phenyl)methanol (385 mg, 2.19 mmol) in DMF (15 mL) was added NaH (60%, 105 mg, 2.63 mmol), the mixture was stirred at 0° C. under N₂ for 0.5 h., and then 3-cyano-4-fluoro-N-methylbenzamide (390 mg, 2.19 mmol) was added. The resulting mixture was stirred at rt for 2 h., concentrated and purified by flash column chromatography on silica gel (ethyl acetate in petroleum ether = 20% v/v) to obtain the target compound 5 (200 mg, yield 27.3%) as solid. LC-MS (ESI) m/z: 335 [M+H]⁺.

### Step 3: Synthesis of 3-(aminomethyl)-N-methyl-4-(3-(trifluoromethyl)benzyloxy)benzamide (Compound 6)

The mixture of 3-cyano-N-methyl-4-(3-(trifluoromethyl)benzyloxy)benzamide (150 mg, 0.45 mmol), Raney nickel (18 mg, 0.05 mmol) and concentrated NH₃H₂O (8 mL) in EtOH (8 mL) and THF (8 mL) was stirred at rt under H₂ (1 atm) for 1 h. The mixture was filtered through celite, the filtrate was concentrated at reduced pressure to leave the crude compound 6 (200 mg, crude) as oil. LC-MS (ESI) m/z: 339 [M+H]⁺.

### Step 4: Synthesis of 3-(acrylamidomethyl)-N-methyl-4-(3-(trifluoromethyl)benzyloxy)benzamide (Compound I-B-04)

To the solution of 3-(aminomethyl)-N-methyl-4-(3-(trifluoromethyl)benzyloxy)benzamide (200 mg, 0.59 mmol) and Et₃ N (119 mg, 1.18 mmol) in THF (10 mL) was added acryloyl chloride (53 mg, 0.59 mmol). The mixture was stirred at 0° C. under N₂ for 1 h., and then purified directly by prep-HPLC to obtain the target compound I-B-04 (20 mg, yield 8.6%) as solid. LC-MS (ESI) m/z: 393.1 [M+H]⁺. ¹H NMR (400 MHz, DMSO-d₆) δ (ppm) 8.50 (t, J = 4.8 Hz, 1H), 8.30 (d, J = 4.0 Hz, 1H), 7.91 - 7.60 (m, 6H), 7.14 (d, J = 8.1 Hz, 1H), 6.33 (dd, J = 17.0, 10.2 Hz, 1H), 6.13 (dd, J = 16.8, 1.6 Hz, 1H), 5.63 (dd, J = 10.1, 1.6 Hz, 1H), 5.34 (s, 2H), 4.41 (d, J = 5.5 Hz, 2H), 2.75 (d, J = 4.3 Hz, 3H).

### Ii-1

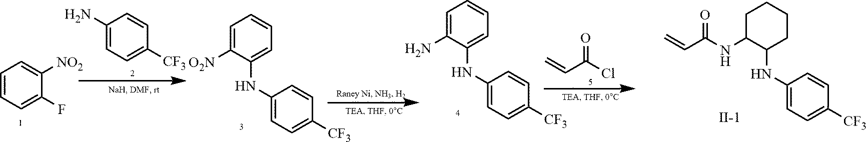

### Step 1: Synthesis of 2-nitro-N-(4-(trifluoromethyl)phenyl)aniline (Compound 3)

To the solution of 4-(trifluoromethyl)aniline (1.25 g, 7.8 mmol) in DMF (15 mL) was added NaH (60%, 340 mg, 14.2 mmol), the mixture was stirred at 0° C. under N₂ for 0.5 h., and then 1-fluoro-2-nitrobenzene (1 g, 7.1 mmol) was added. The resulting mixture was stirred at rt for 16 h., diluted with EtOAc (100 mL), washed with brine (50 mL), dried over anhydrous Na₂SO₄, concentrated and purified by flash column chromatography on silica gel (ethyl acetate in petroleum ether = 50% v/v) to obtain the target compound 3 (500 mg, yield 25.0%) as solid. LC-MS (ESI) m/z: 283 [M+H]⁺.

### Step 2: Synthesis of Nl-(4-(trifluoromethyl)phenyl)benzene-1,2-diamine (Compound 4)

To the mixture of 2-nitro-N-(4-(trifluoromethyl)phenyl)aniline (450 mg, 1.6 mmol) and Raney nickel (65 mg, 0.16 mmol) in EtOH (10 mL) and THF (10 mL) was added N₂H₄●H₂O (200 mg, 4.0 mmol). The mixture was stirred at rt for 1 h and filtered, the filtrate was concentrated at reduced pressure to leave the crude compound 4 (400 mg, crude) as oil. LC-MS (ESI) m/z: 253 [M+H]⁺.

### Step 3: Synthesis of N-(2-(4-(trifluoromethyl)phenylamino)phenyl)acrylamide (Compound II-1)

To the solution of N1-(4-(trifluoromethyl)phenyl)benzene-1,2-diamine (350 mg, 1.39 mmol) and Et₃ N (281 mg, 2.78 mmol) in THF (20 mL) was added acryloyl chloride (125 mg, 1.39 mmol). The mixture was stirred at 0° C. under N₂ for 1 h and then concentrated in vacuum, the residue purified by prep-HPLC to obtain the target compound II-1 (188 mg, yield 44.2%) as solid. LC-MS (ESI) m/z: 307.1 [M+H]⁺. ¹H NMR (400 MHz, DMSO-d₆) δ (ppm) 9.61 (s, 1H), 8.00 (s, 1H), 7.72 (d, J = 7.4 Hz, 1H), 7.46 (d, J = 8.5 Hz, 2H), 7.33 (d, J = 7.6 Hz, 1H), 7.15 (dt, J = 15.1, 6.9 Hz, 2H), 6.90 (d, J = 8.5 Hz, 2H), 6.48 (dd, J = 17.0, 10.2 Hz, 1H), 6.23 (dd, J = 17.0, 1.5 Hz, 1H), 5.72 (d, J = 10.1 Hz, 1H).

### Ii-2

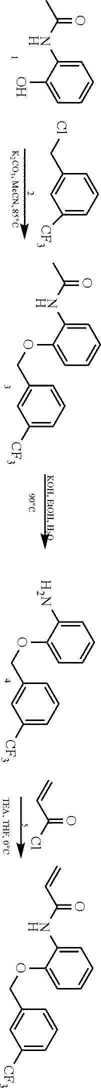

### Step 1: Synthesis of N-(2-(3-(trijluoromethyl)benzyloxy)phenyl)acetamide (Compound 3)

The mixture of 1-(chloromethyl)-3-(trifluoromethyl)benzene (1.41 g, 7.28 mmol), N-(2-hydroxyphenyl)acetamide (1 g, 6.62 mmol) and K₂CO₃ (2.74 g, 19.86 mmol) in MeCN (20 mL) was stirred at 85° C. under N₂ for 16 h. The mixture was filtered, the filtrate was concentrated and purified by flash column chromatography on silica gel (ethyl acetate in petroleum ether = 50% v/v) to obtain the target compound 3 (1.5 g, yield 73.5%) as solid. LC-MS (ESI) m/z: 310 [M+H]⁺.

### Step 2: Synthesis of 2-(3-(trifluoromethyl)benzyloxy)aniline (Compound 4)

The mixture of N-(2-(3-(trifluoromethyl)benzyloxy)phenyl)acetamide (500 mg, 4.62 mmol) and KOH (272 mg, 4.86 mmol) in EtOH (10 mL) and H₂O (10 mL) was stirred at 90° C. under N₂ for 16 h. The mixture was diluted with EtOAc (100 mL), washed with brine (50 mL), dried over anhydrous Na₂SO₄, filtered and concentrated to leave the crude compound 4 (400 mg, crude) as oil. LC-MS (ESI) m/z: 268 [M+H]⁺.

### Step 3: Synthesis of N-(2-(3-(trifluoromethyl)benzyloxy)phenyl)acrylamide (Compound II-2)

To the solution of 2-(3-(trifluoromethyl)benzyloxy)aniline (50 mg, 0.19 mmol) and Et₃ N (38 mg, 0.38 mmol) in THF (10 mL) was added acryloyl chloride (17 mg, 0.19 mmol). The mixture was stirred at 0° C. under N₂ for 1 h., and then concentrated and purified by prep-HPLC to obtain the target compound 11-2 (95 mg, yield 22.6%) as solid. LC-MS (ESI) m/z: 322.1 [M+H]⁺. ¹H NMR (400 MHz, DMSO-d₆) δ (ppm) 9.47 (s, 1H), 7.90 (s, 2H), 7.80 (d, J = 7.5 Hz, 1H), 7.65 (dt, J = 15.3, 7.7 Hz, 2H), 7.21 - 7.07 (m, 2H), 6.98 - 6.90 (m, 1H), 6.66 (dd, J = 17.0, 10.2 Hz, 1H), 6.25 (dd, J = 17.0, 1.9 Hz, 1H), 5.74 (dd, J = 10.2, 1.9 Hz, 1H), 5.32 (s, 2H).

### Example 2. Assays Analyzing Combined EGFR and MEK Inhibition With Inhibition of TEAD, Tumor Dormancy, and TEAD Inhibition

In the current study, the mechanisms that allow cancer cells to evade apoptosis and survive despite combined EGFR/MEK inhibition were studied.

### Combined EGFR and MEK Inhibition Results in a Stable, but Reversible Dormant State

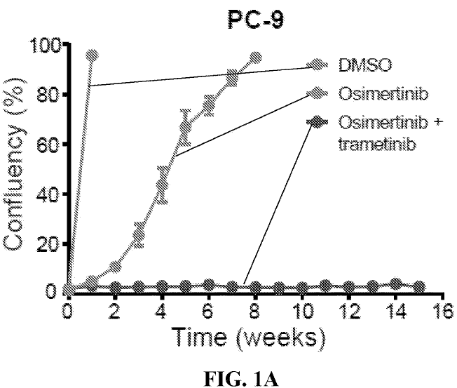

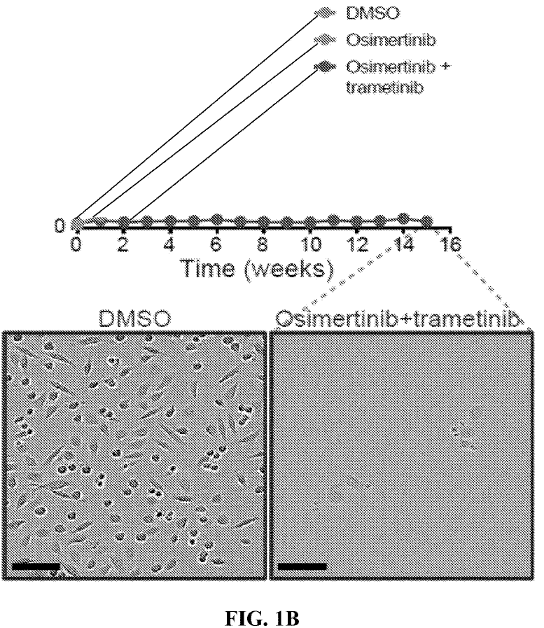

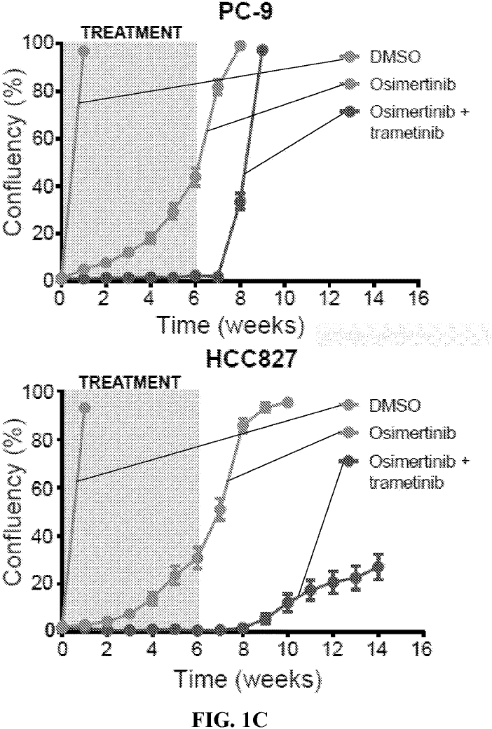

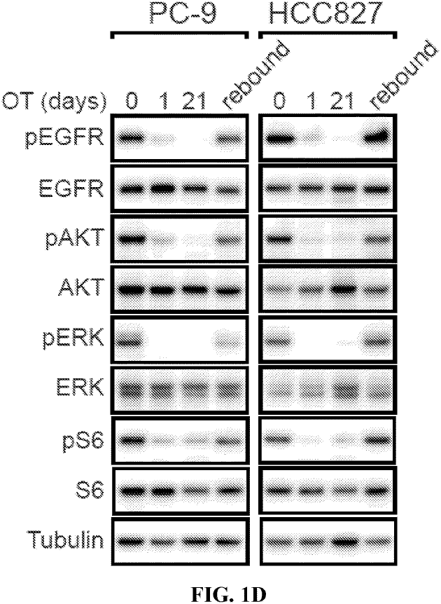

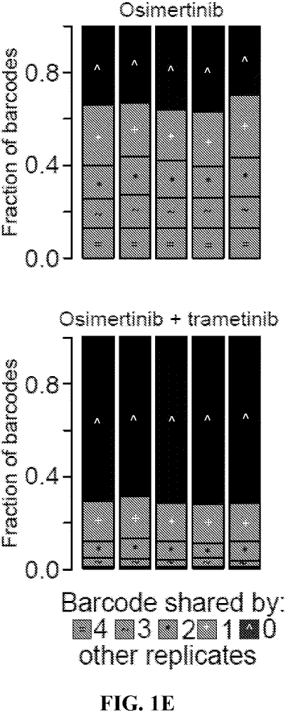

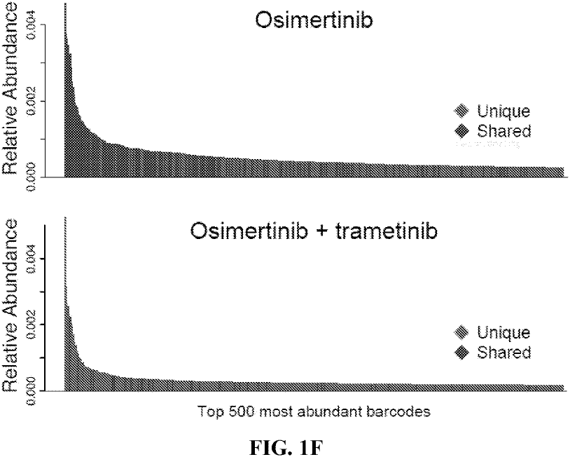

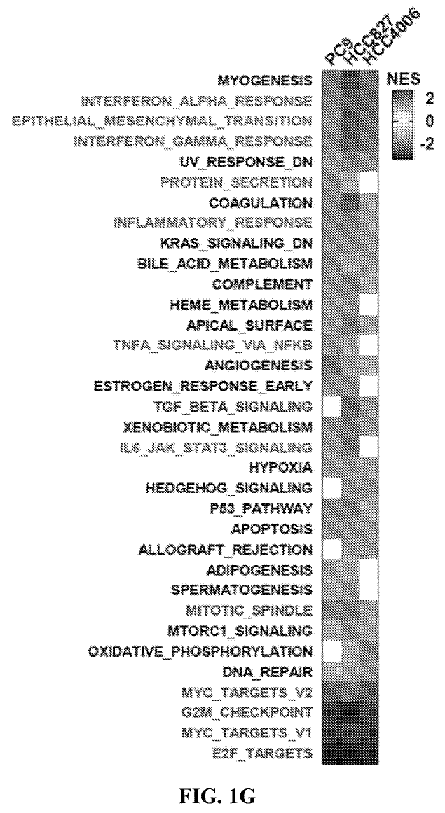

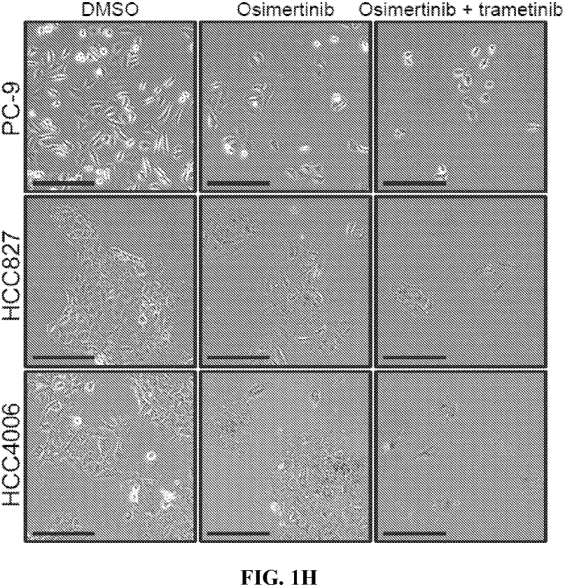

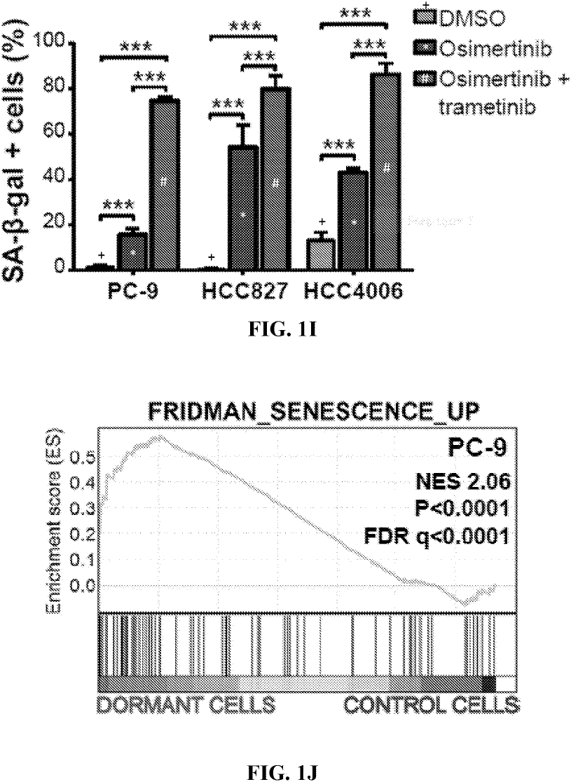

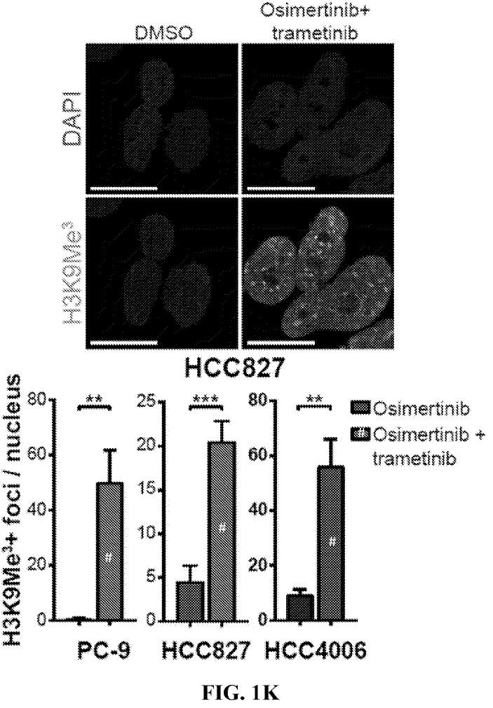

Combined EGFR/MEK inhibition prevents the re-activation of ERK½ following EGFR inhibition and delays the onset of drug resistance in EGFR-mutant NSCLC models in vitro and in vivo (Tricker et al., 2015) (FIG. 8A). In PC-9 cells, continuous drug treatment with single-agent osimertinib leads to re-colonization of wells within 8 weeks (FIG. 1A). The combination of osimertinib and the MEK inhibitor trametinib, however, prevents any measurable regrowth for>15 weeks of treatment (FIG. 1A). Remarkably, few viable cells can still be detected after 15 weeks of treatment (FIG. 1B). Live cell imaging was used over several weeks and observed that the osimertinib/trametinib treated cells surviving the initial wave of apoptosis remained in a largely non-proliferative, or dormant, state throughout the drug treatment period. For FIG. 1C, PC-9 cells were treated with a combination of osimertinib and trametinib for 25 days followed by drug washout. Cells were imaged every 2 hours for a total of 40 days using the Incucyte FLR live-cell imaging system. However, within days following drug withdrawal these cells begin to migrate, proliferate and re-colonize the wells (FIG. 1C). This phenomenon was consistent across EGFR mutant NSCLC cell lines (FIG. 1C and FIG. 8B). These observations suggest that while combined EGFR/MEK inhibition eliminates cells where re-activation of ERK signaling occurs following single-agent EGFR inhibition, a separate population of cells can enter a dormant state, surviving even in the presence of combined EGFR/MEK inhibition. Consistent with the stable, but reversible non-proliferative state, there was no evidence of re-activation of EGFR and/or ERK signaling in the dormant cells during treatment (FIG. 1D), and EGFR signaling was restored in cells that grew following drug washout (FIG. 1D). These cells were still sensitive to osimertinib/trametinib, and morphologically indistinguishable from the untreated cells (FIG. 8C) suggesting that a subclone with a pre-existing resistance mutation was not selected out (Hata et al., 2016). To formally address whether the establishment of dormancy following osimertinib/trametinib is pre-determined or a stochastic process, PC-9 cells were barcoded using the EvoSeq library (Feldman et al., 2019) with a complexity of ~500 000 barcodes, treated the cells with DMSO, gefitinib, osimertinib and osimertinib/trametinib, sequenced DNA from the remaining cells following 3 weeks of drug treatment and analyzed the findings as described (Bhang et al., 2015) with some modifications (described in the methods section). A large fraction of shared barcodes in the gefitinib was observed (data not shown) and osimertinib (FIG. 1E) treated cells, strongly suggesting selection of pre-existing clones which is consistent (for gefitinib) with prior studies (Hata et al., 2016). In contrast, the vast majority of barcodes in the osimertinib/trametinib -treated cells are unique (FIG. 1E). However, there is also a small fraction of shared barcodes within the osimertinib/trametinib treated cells (FIG. 1F, FIG. 9A). Comparison of the shared barcodes between osimertinib and osimertinib/trametinib cells demonstrate that 89% of the barcodes identified in the osimertinib treated cells are not present in the osimertinib/trametinib treated cells (FIG. 9B). These findings suggest that while resistance to osimertinib likely occurs through a selection process of a pre-existing clone, the ability of cells to enter dormancy following osimertinib/trametinib is predominately driven by a stochastic process with some contribution from a selection process.

### Dormant State Following EGFR/MEK Inhibition Shares Characteristics with Cellular Senescence

In order to characterize the dormant state, RNA-sequencing (RNA-seq) in PC-9, HCC827, and HCC4006 cells was performed following treatment with either DMSO or with osimertinib/trametinib for two weeks. Gene set enrichment analysis (GSEA) revealed an up-regulation of gene expression signatures involved in inflammatory response, epithelial-to-mesenchymal transition (EMT) and protein secretion while cell cycle -associated gene expression signatures were robustly down-regulated (FIG. 1G). These findings along with the spread-out, flattened morphology of the dormant cells (FIG. 1B) prompted consideration of the possibility that the dormant state could resemble cellular senescence. DMSO, osimertinib or osimertinib/trametinib-treated PC-9, HCC827 and HCC4006 cells were stained for senescence-associated (β-galactosidase activity (SA-β-Gal) (Debacq-Chainiaux et al., 2009), and noted that for all 3 cell lines, the majority of cells undergoing the combination treatment stained positive for SA-(β-Gal (FIGS. 1H-1I). Further GSEA analysis also revealed a significant enrichment of a senescence-associated gene expression signature (Fridman and Tainsky, 2008) in the dormant cells derived from all three cell lines (FIG. 1J, FIG. 10A). Of note, a significantly smaller proportion of single-agent osimertinib-treated cells demonstrated SA-(β-Gal activity compared to those treated with the trametinib combination (FIGS. 1H-1I). Senescent cells also characteristically exhibit increased secretion of pro-inflammatory factors (senescence-associated secretory phenotype, SASP) (Coppé et al., 2010). By analyzing conditioned medium from dormant cells derived from PC-9, HCC827 and HCC4006 cells, an increased secretion of several cytokines and chemokines, including the prominent SASP factor IL-6, compared to untreated cells was observed (FIG. 10B). The expression of several classical SASP factors (Coppé et al., 2008) in the cytokine, chemokine, IGFBP and MMP families was also upregulated in the dormant cells (FIG. 10C), further reminiscent of the SASP.

Another hallmark of senescence is extensive epigenetic remodeling, including the appearance of distinct, H3K9Me³-positive nuclear foci (senescence-associated heterochromatic foci, SAHF) (Narita et al., 2003). Immunofluorescence (IF) was used to evaluate the pattern of H3K9Me³ distribution in chromatin. Ten-day treatment with osimertinib/trametinib resulted in a robust increase in punctate, H3K9Me³-positive nuclear foci in PC-9, HCC827 and HCC4006 cells (FIG. 1K), indicating senescence-like epigenetic remodeling in the dormant cells. Establishment of senescence is also invariably associated with the induction of p16^{INK4a}, p21^{Cip1} and/or p27^{Kip} (Campisi and D’Adda Di Fagagna, 2007). Although consistent p16^{INK4a} or p21^{Cip1} induction was not observed, a robust induction of p27^{Kip} which was subsequently downregulated in growing cells following drug withdrawal was, as shown in FIG. 10D.

### The Establishment of Dormancy Following EGFR/MEK Inhibition Is Critically Dependent on Activation of YAP/TEAD

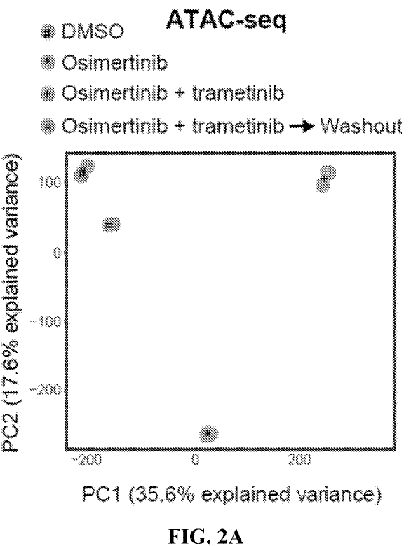

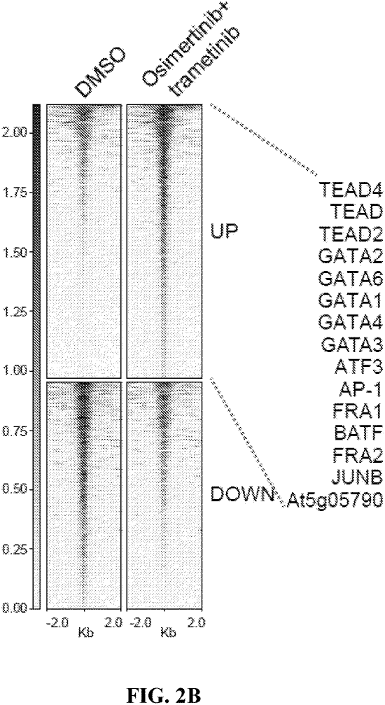

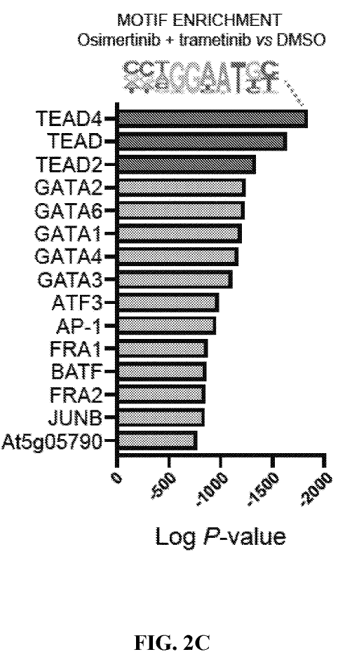

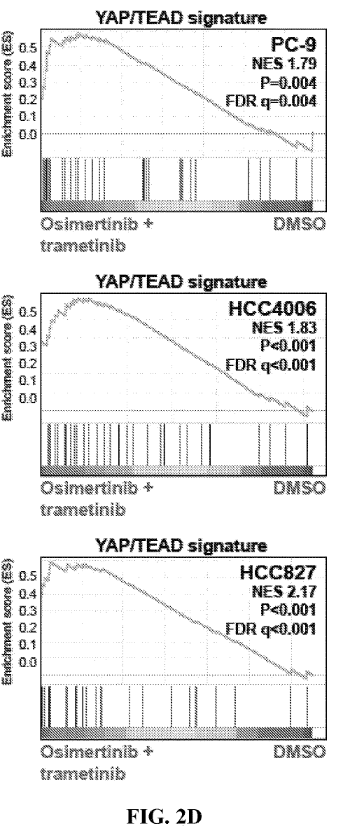

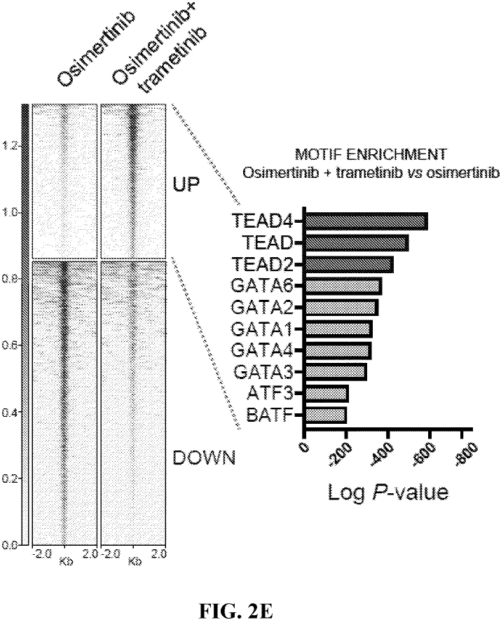

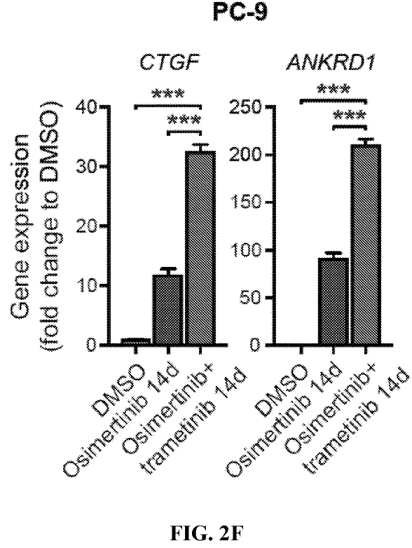

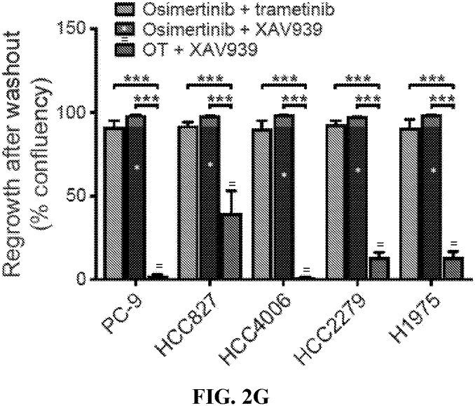

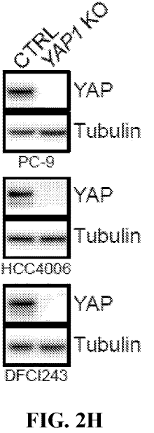

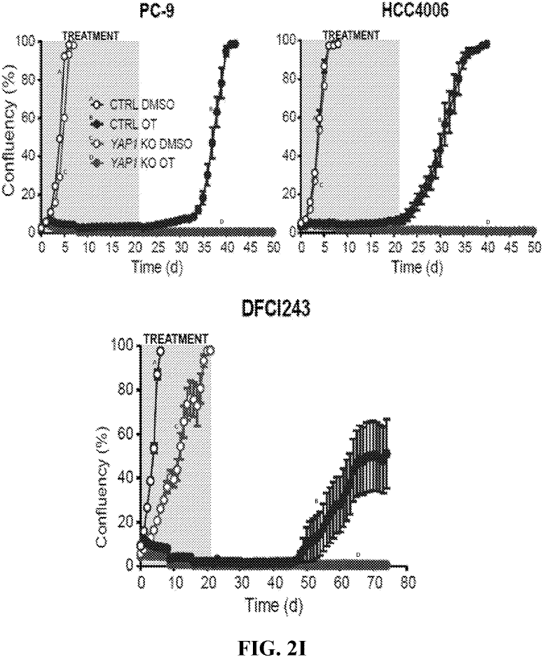

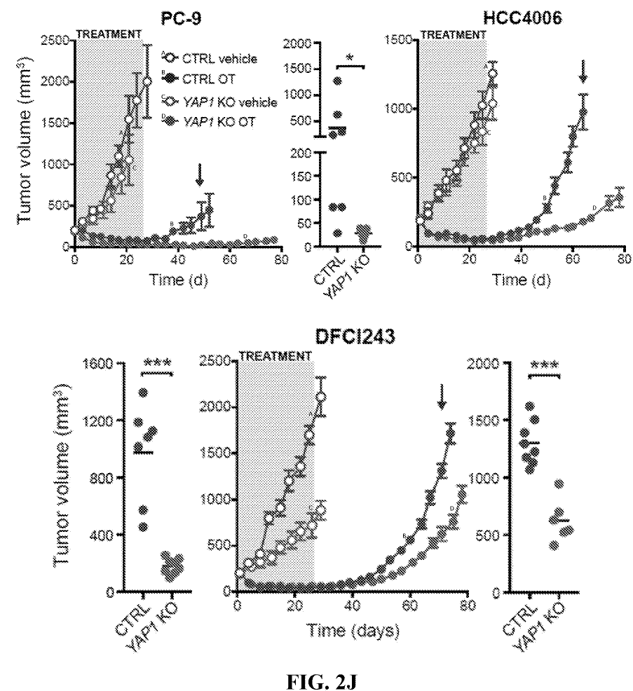

Since the data suggested that the senescence-like dormant state is regulated by non-mutational mechanisms, the epigenetic changes in the dormant cells were explored. Using an assay for transposase-accessible chromatin combined with next-generation sequencing (ATAC-seq), a significant difference in the global epigenetic states between the osimertinib/trametinib-induced dormant vs. DMSO-treated control cells was observed (FIG. 2A). This epigenetic state was reverted back upon drug washout, demonstrating that the changes acquired at dormancy are reversible (FIG. 2A). A significant difference in epigenetic states between single-agent osimertinib and osimertinib/trametinib -induced dormant cells was also observed (FIG. 2A). A motif analysis was performed to interrogate transcription factor binding sites associated with the ATAC-seq peaks with higher signal (more accessible chromatin) in osimertinib/trametinib -induced dormant cells (FIG. 2B). Intriguingly, the three most significantly enriched motifs were the consensus sites for TEAD family transcription factors (FIG. 2C), suggesting that the osimertinib/trametinib -induced epigenetic state is associated with increased TEAD transcription factor binding. The TEAD transcription factors serve as canonical partners for the Hippo pathway effector YAP, which has been associated with resistance to targeted therapy in several contexts, including in resistance to EGFR TKI’s in EGFR-mutant NSCLC (Chaib et al., 2017; Hsu et al., 2016). Indeed, a significant enrichment of a previously published YAP/TEAD gene expression signature was observed (Zhang et al., 2009) in the dormant PC -9, HCC4006 and HCC827 cells vs control cells (FIG. 2D). Furthermore, the TEAD binding motifs also scored as the most significant top hits separating the osimertinib/trametinib, and osimertinib treatment induced states (FIG. 2E). In accordance with these findings, significantly higher YAP/TEAD activity was observed, as measured by CTGF and ANKRDl expression, in osimertinib/trametinib -induced dormant PC-9 cells compared to osimertinib-treated cells (FIG. 2F). Consistently, increased chromatin accessibility at putative distal enhancer sites upstream of CTGF TSS in osimertinib/trametinib-induced dormant cells compared to osimertinib alone treated cells was detected (FIG. 11A). Taken together, these results demonstrate that dormant cells induced by combined EGFR/MEK inhibition adopt a distinct, reversible epigenetic state set distinct from the untreated or the osimertinib-treated state by increased YAP/TEAD activity.

To investigate the importance of YAP activity for the establishment of osimertinib/trametinib -induced dormant state, EGFR-mutant NSCLC cell lines were treated for three weeks with osimertinib/trametinib with or without the tankyrase inhibitor XAV939, an indirect inhibitor of YAP activity (Wang et al., 2015; see References below), and assessed the ability of XAV939 to prevent the regrowth of cells after drug washout. Remarkably, XAV939 led to a significant reduction in regrowth in all cell lines when combined with osimertinib/trametinib (FIG. 2G). This reduction was due to the dramatic decrease in the number of dormant cells, demonstrated by directly counting viable cells at the time of drug washout (FIG. 11B). Similar findings were made with three additional structurally divergent tankyrase inhibitors (FIG. 11B). The effect of several inhibitors targeting putative resistance pathways to EGFR TKI’s, or the effect of chemotherapy in PC-9 cells, was tested, but little to no effect on the establishment of the dormant cell population was observed (FIGS. 11C-11D). Notably, the combination of single-agent osimertinib and XAV939 was significantly inferior to the osimertinib/trametinib/XAV939 combination in all EGFR-mutant NCSLC cell lines tested (FIG. 2G), suggesting that the differences seen in YAP/TEAD activity between osimertinib/trametinib-induced dormant cells and single-agent osimertinib-treated cells may reflect different degrees of dependency on YAP.

To further validate the role of YAP in the establishment of the dormant state, YAP1 was knocked out in three different EGFR-mutant NSCLC cell lines, including a patient-derived cell line DFCI243, using the CRISPR/CAS9 system (FIG. 2H). Strikingly, YAP1 knock-out (KO) completely abolished the establishment of dormant cells in all three cell lines, measured by lack of regrowth following drug withdrawal after a three-week treatment with osimertinib/trametinib combination (FIG. 2I). In contrast, following osimertinib alone, regrowth was eventually observed in 2 out of 3 YAP1 KO cell lines (FIG. 11E). The impact of YAPl KO in vivo was evaluated. Combined osimertinib/trametinib treatment of mice bearing xenograft tumors from PC-9, HCC4006 and DFCI243 YAP1 KO cells or from the corresponding control cells led to a durable response for the whole four-week drug treatment period (FIG. 2J). However, the control tumors started to regrow soon after treatment cessation, consistent with the presence of a dormant cell population in vivo. In contrast, YAP1 KO tumors had an increased latency in tumor regrowth and significantly smaller tumors at the time of regrowth in all xenograft models (FIG. 2J), consistent with a reduction in the dormant cell population. Collectively, these results demonstrate that the establishment of drug tolerant state following EGFR/MEK inhibition, but not single-agent EGFR inhibition, is critically dependent on YAP/TEAD activity.

### YAP Activation Is Necessary for Cancer Cell Viability Upon Combined EGFR/MEK Inhibition

In order to further assess the dynamics of YAP/TEAD activity following drug treatment, a fluorescent YAP/Hippo pathway reporter was introduced (Mohseni et al., 2014) into PC-9 cells (PC-9 YAP reporter cells) and used Incucyte live-cell imaging to track YAP activity over time. It was observed that osimertinib/trametinib treatment induces a robust increase in YAP activity, indicating that YAP is activated as a response to drug treatment (FIG. 3A). This treatment-induced increase in YAP activity was completely blocked by the addition of XAV939 (FIG. 3A). Consistent with the treatment induced increase in YAP, decreased phosphorylation of the main YAP upstream negative regulator, LATS1, and a decrease in YAP S 127 phosphorylation which regulates YAP cytosolic retention was noted (FIG. 12). Consequently, YAP nuclear localization was significantly increased in EGFR-mutant NCSLC cells following a 10-day treatment with osimertinib/trametinib, but not following single-agent osimertinib-treatment (FIG. 3B), consistent with the more prominent increase in YAP activity observed in the combination-treated cells (FIG. 2F and FIG. 3A).

The activation of YAP in response to treatment suggested that YAP activity protects cells from the initial apoptosis. To evaluate this possibility, non-invasive monitoring of caspase 3/7 activity was used over time in the PC-9 YAP reporter cells. The increase in YAP activation occurs proportionally to apoptosis (FIG. 3C) - however, the fluorescent signals from caspase 3/7 activation and YAP activation appeared to occur in different cells (FIG. 3D). Indeed, it was noted that cells with high YAP activity (YAP^{high} cells) were significantly less likely to undergo apoptosis (FIG. 3D; odds ratio 0.26 at 80 h, p<0.0001 by Fisher’s exact test). In accordance with these observations, a robust increase in apoptosis was noted when EGFR-mutant NSCLC cells were treated with XAV939 together with osimertinib/trametinib compared to osimertinib/trametinib alone or to osimertinib/XAV939 (FIGS. 3E-3F). Analogously, the YAP1 knock-out cells underwent highly increased and accelerated apoptosis in response to osimertinib/trametinib (FIG. 3G). Importantly, this hypersensitive phenotype was rescued by the re-expression of wild-type YAP1 in the YAP1 KO cells (FIG. 3H).

The combination of osimertinib and trametinib promoted significantly higher YAP reporter activity than single-agent osimertinib (FIG. 3A), suggesting that the differences in YAP/TEAD activity seen at drug tolerant state (FIGS. 2E-2F) reflect the cells’ immediate responses to the different drug treatments. As the main consequence of concomitant MEK inhibition is the suppression of ERK½ re-activation following EGFR inhibition, the results suggest a hypothesis that ERK½ re-activation and YAP activation are two separate means by which EGFR-mutant NSCLC cells evade apoptosis following single agent osimertinib treatment, and preventing ERK½ re-activation by concomitant MEK inhibition, only cells that activate YAP survive. In support of this hypothesis, the proportion of YAP^{high} cells in single-agent osimertinib- and osimertinib/trametinib -treated populations was quanitifed using the PC-9 YAP reporter cells. Following a 10-day treatment, the osimertinib-treated cell population contained both YAP^{high} cells (40%) and cells with absent YAP signal (60%), whereas in the osimertinib/trametinib-treated population the cells with absent YAP activity were largely depleted, and most (> 80%) of the surviving cells were YAP^{high} cells (FIG. 3I). Taken together, these observations demonstrate that chronic downregulation of EGFR- and its downstream signaling by concomitant EGFR and MEK inhibition selects for cells that induce high YAP activity upon treatment, creating a vulnerability that can be exploited to selectively promote apoptosis in these cells through simultaneous inhibition of YAP (FIG. 3J).

### YAP-High, Senescence-Like Dormant State Also Occurs in Vivo

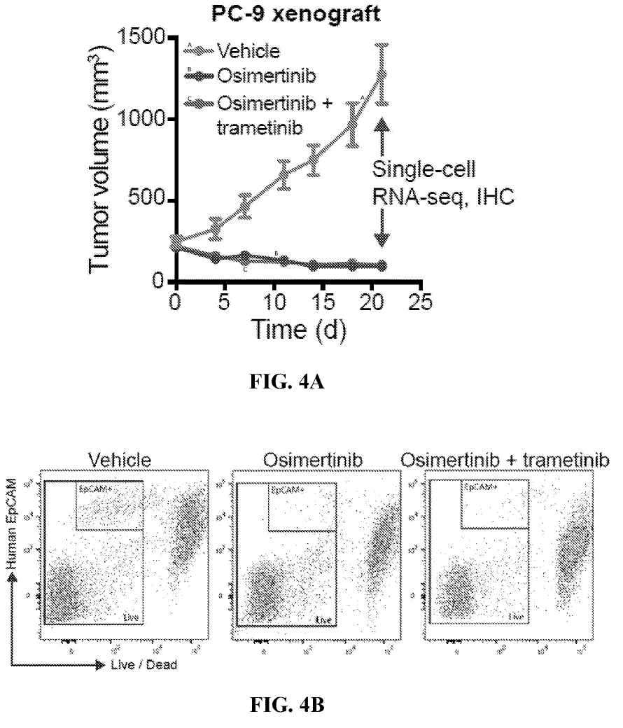

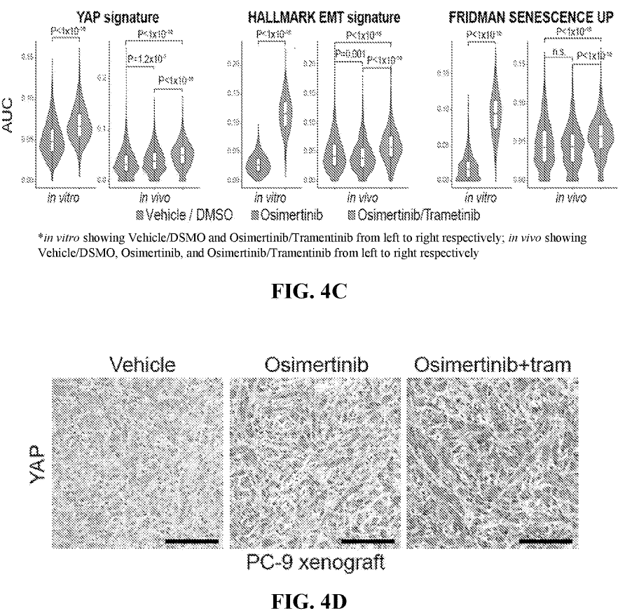

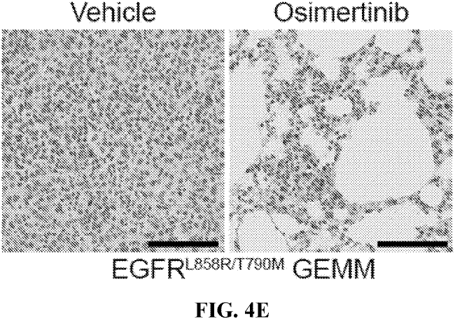

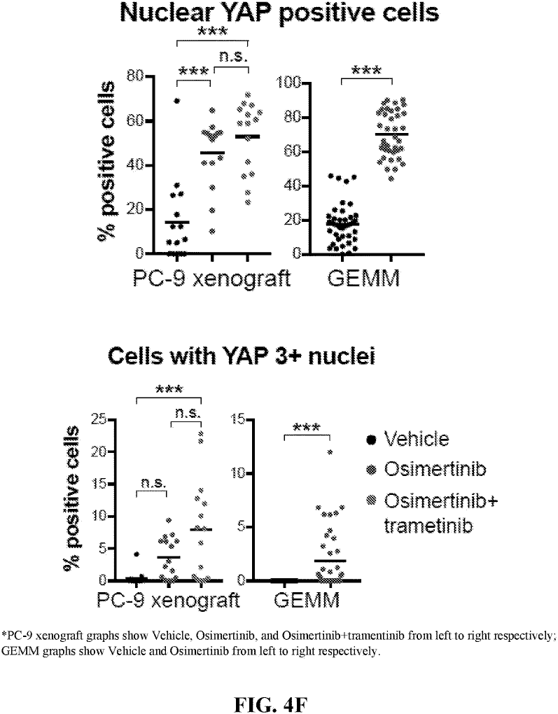

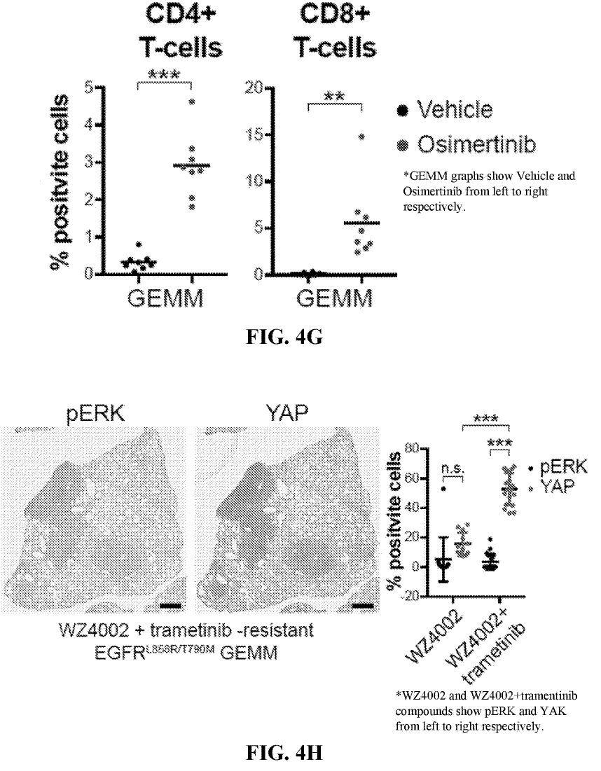

In order to determine whether the senescence-like dormant state also occurs in vivo, single-cell RNA-sequencing (scRNA-seq) was performed from PC-9 xenograft tumors treated with vehicle, osimertinib or with osimertinib/trametinib until minimal residual disease (MRD) was reached (3 weeks) (FIGS. 4A-4B; FIG. 13A). scRNA-sequencing from dormant PC-9 cells was also performed following 3 weeks of osimertinib/trametinib treatment in vitro. A significant increase in cells enriched for YAP, EMT or senescence gene expression signatures in the osimertinib/trametinib treated PC-9 cells in vitro and in vivo was detected (FIG. 4C). YAP expression and subcellular localization from the same PC-9 MRD in vivo samples using immunohistochemistry was also analyzed (IHC). Consistent with the scRNA-seq studies, an increase in YAP nuclear localization in the MRD tumors, with more intense nuclear staining in the combination-treated tumors was detected (FIG. 4D). MRD tumors were further evaluated from genetically engineered EGFR^{L858R/T790M} mice following 2 weeks of osimertinib treatment and similarly noted an increased in YAP nuclear localization (FIGS. 4E and 4F (Zhou et al., 2009). As these mice have an intact immune system, T-cell infiltration was observed. An increase in infiltration of CD4+ and CD8+ T-cells was observed (FIG. 4G, FIG. 13B), suggesting that the MRD tumors elicit an immune response, consistent with the findings of an increase in secreted inflammatory factors (FIGS. 9B-9C) and with prior studies in lung cancer patients (Thress et al., 2017). However, despite of the T-cell response, neither osimertinib- nor osimertinib/selumetinib (a clinical MEK inhibitor under phase II study with osimertinib; NCT03392246) treated EGFR^{L858R/1790M} mice could be cured by the treatment (FIG. 13C) suggesting that the immune response is insufficient to eradicate the YAP-high residual cells. Similarly, no patient with advanced EGFR mutant lung cancer has been cured with osimertinib.

Tumors from EGFR^{L858R/T790M} mice were further studied that had developed acquired resistance to WZ4002 (preclinical 3^{rd} generation EGFR inhibitor)/trametinib combination from the prior study (Tricker et al., 2015; see References below). In agreement with the model that YAP can sustain the viability of cancer cells in the absence of EGFR downstream signaling, robust YAP nuclear staining and a lack of pERK ½ expression in the resistant tumor nodules was detected (FIG. 4H). A significantly higher proportion of nuclear YAP-positive cells in WZ4002/trametinib-resistant nodules compared to single-agent WZ4002-resistant nodules was observed (FIG. 4H), consistent with the in vitro observations (FIGS. 2A-2J and FIGS. 3A-3J). Finally, YAP nuclear staining and pERK ½ expression from an EGFR mutant patient treated with osimertinib/selumetinib (NCT03392246) who had a sustained partial response was analyzed. The patient underwent surgery following 11 months of treatment while in a clinical MRD state. The residual tumor demonstrated intense YAP staining and an absence of pERK ½ staining (FIG. 4I).

### YAP Mediates the Evasion of Apoptosis by Repressing the Induction of the Pro-Apoptotic Protein BMF

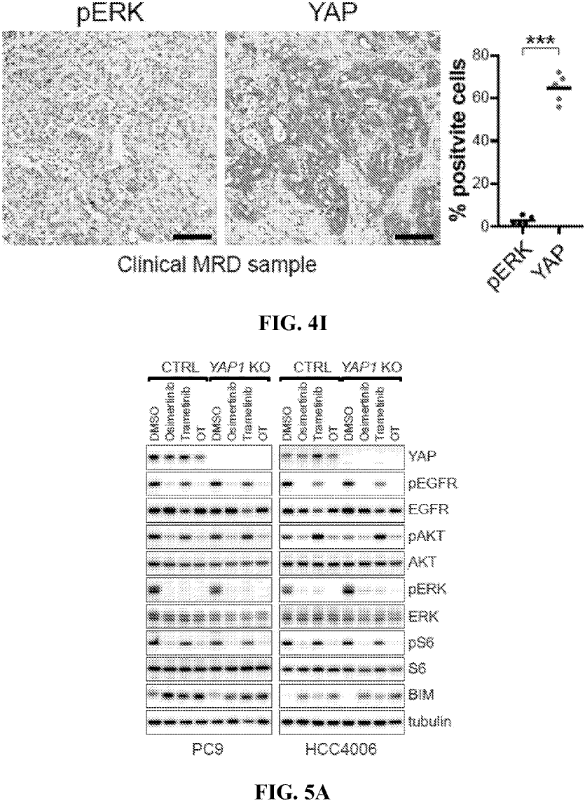

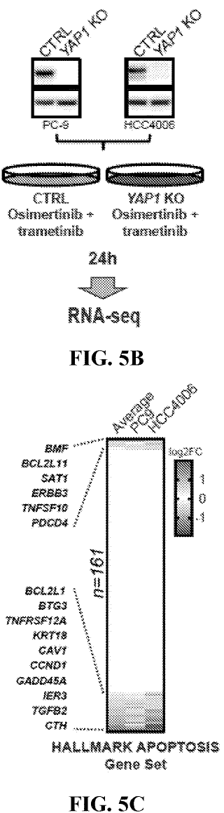

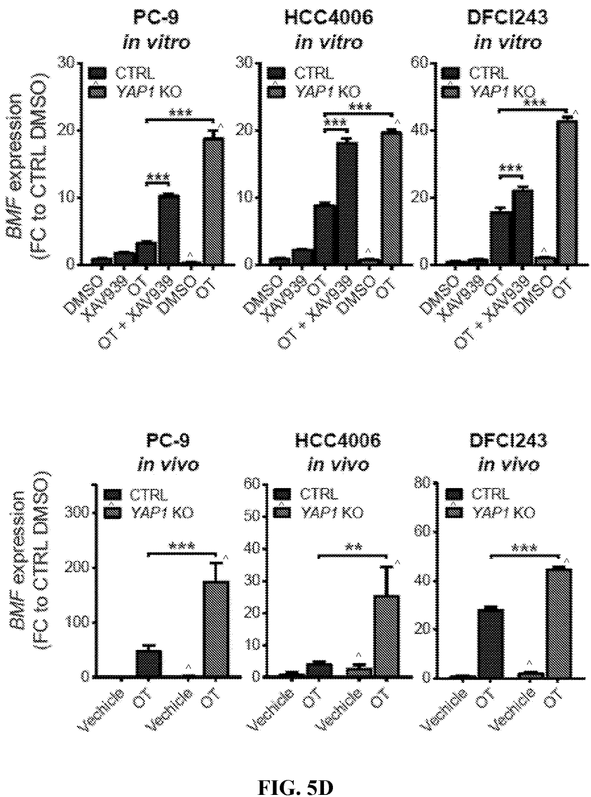

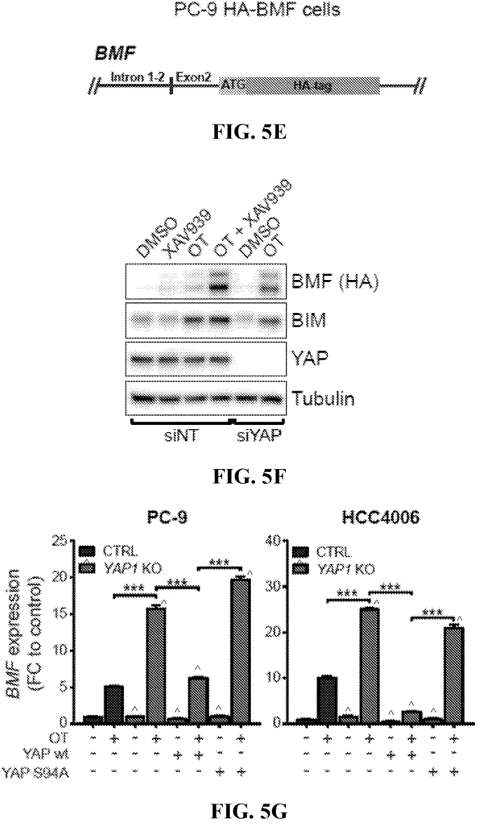

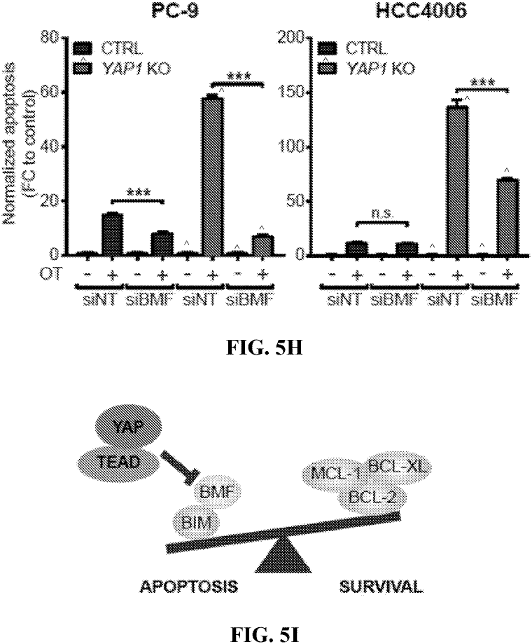

The mechanism by which YAP protects EGFR-mutant NSCLC cells from apoptosis was elucidated. YAP1 KO had no effect on canonical EGFR signaling or on the induction of BIM protein, a known mediator of apoptosis following EGFR inhibition (Costa et al., 2007; Cragg et al., 2007; Gong et al., 2007), in response to osimertinib alone, trametinib alone or to the combination (FIG. 5A). This suggests that YAP affects the apoptotic process independently of EGFR signaling and downstream of BIM. YAP has been shown to regulate the expression of anti-apoptotic proteins including BCL-XL (Rosenbluh et al., 2012), and this has been proposed to be the mechanism underlying the protective effect of YAP in the context of mutant RAS- or BRAF-driven cancers (Lin et al., 2015). However, no significant changes in the levels of anti-apoptotic proteins BCL-XL, BCL2, BCL-w or MCL-1 in YAP1 KO cells compared to control cells at baseline or following osimertinib/trametinib was observed (FIG. 14A). In contrast, a substantial increase in BAX activity (FIG. 14B), cytochrome c release (FIG. 14C) and caspase activation (FIG. 3G) in YAP1 KO cells in response to osimertinib/trametinib was observed, suggesting that the increased apoptosis seen in YAP1 KO cells is mediated by the intrinsic apoptotic pathway.

To identify potential YAP target gene(s), RNA-sequencing was performed on PC-9 and HCC4006 YAP1 KO or control cells with and without osimertinib/trametinib treatment (FIG. 5B). Focusing on genes that mediate apoptosis through the activation of caspases (Hallmark Apoptosis gene set, 161 genes) (Liberzon et al., 2015), BMF was identified as one of the top up-regulated genes in drug treated YAP1 KO cells compared to drug treated control cells in both cell lines (FIG. 5C). The BMF gene encodes a pro-apoptotic BH3-only protein that can sequester the anti-apoptotic proteins, but unlike pro-apoptotic activators such as BIM, cannot directly activate BAX or BAK (Bhola and Letai, 2016; Kuwana et al., 2005). Together with BIM, expression of which is induced following EGFR inhibition (FIG. 4A), an increase in BMF expression in YAP1 KO cells would be expected to lead to increased activation of BAX and thus to enhanced apoptosis, which is consistent with all of the observations (FIG. 3G and FIGS. 14B-14C). The RNA-sequencing results using QPCR was confirmed; YAP inhibition by XAV939 or YAP1 knock-out significantly increased BMF expression in response to osimertinib/trametinib in three different EGFR-mutant NSCLC cell lines in vitro and in vivo (FIG. 5D). Due to lack of high-affinity antibodies for BMF, CRISPR/CAS9 technology was used to introduce an HA-tag to the endogenous PC-9 BMF locus directly following the BMF start codon, producing N-terminally tagged BMF protein under the endogenous promoter (FIG. 5E, FIGS. 14A-14G). In these cells, YAP inhibition or YAP1 knock-down led to a robust increase in BMF protein levels, while BIM levels remained unchanged (FIG. 5F). Moreover, re-introduction of wild-type YAP, but not TEAD-binding deficient S94A mutant to the YAP1 KO background completely abolished the increase in BMF expression (FIG. 5G), further demonstrating that BMF levels are suppressed by YAP through TEAD in response to osimertinib/trametinib treatment. Ectopic overexpression of BMF, using a doxycycline-inducible vector in EGFR-mutant NSCLC cells (FIG. 14E), induced rapid apoptosis, which was further increased by co-treatment with osimertinib/trametinib, (FIG. 14F), demonstrating that induction of BMF alone, without YAP activation, is sufficient to sensitize EGFR-mutant NSCLC cells to apoptosis. Finally, repression of BMF expression using siRNA significantly decreased apoptosis in YAP1 KO cells in response to osimertinib/trametinib (FIG. 5H and FIG. 14G), demonstrating that the induction of BMF expression is necessary for the increased apoptosis in YAP1 KO cells. These data demonstrate that YAP facilitates evasion of apoptosis in EGFR-mutant NSCLC cells by repressing the expression of the pro-apoptotic protein BMF upon combined EGFR/MEK inhibition, thus leading to survival of cells that subsequently establish the dormant cell population (FIG. 5I).

### YAP Represses BMF Induction by Engaging EMT Transcription Factor SLUG

Next, the molecular mechanisms by which YAP represses the expression of BMF were investigated. Although YAP is mostly associated with transcriptional activation, it has also been shown to complex with transcription factors and transcription modulators to drive transcriptional repression, often in complex with TEAD (Beyer et al., 2013; Kim et al., 2015; Zaidi et al., 2004). Thus, it was hypothesized that YAP/TEAD complex is directly repressing BMF by forming a tertiary complex with a transcriptional repressor.

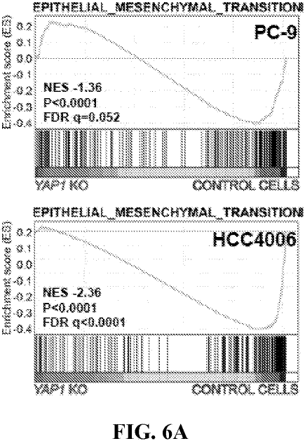

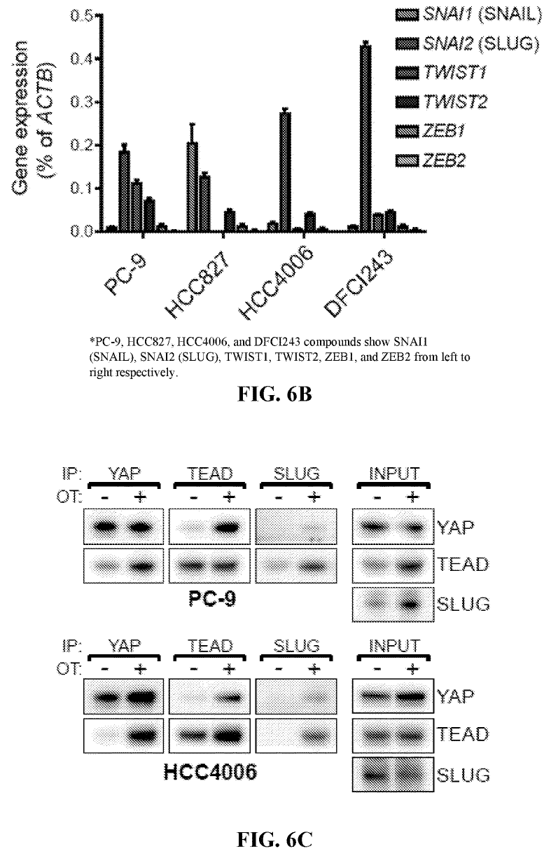

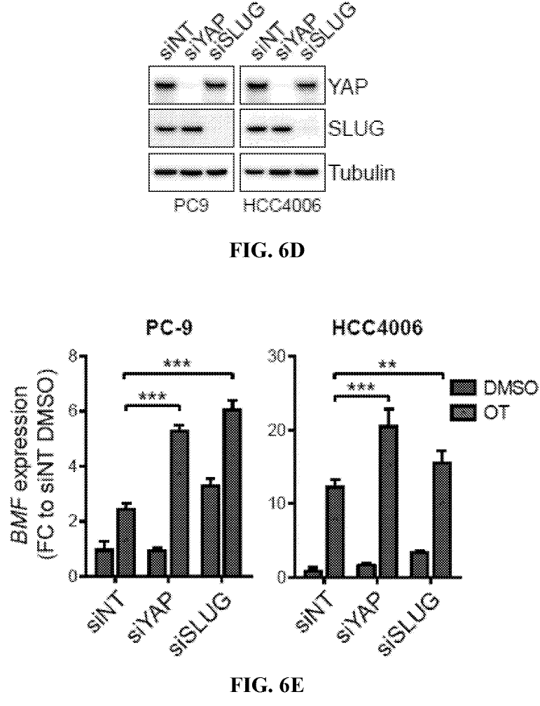

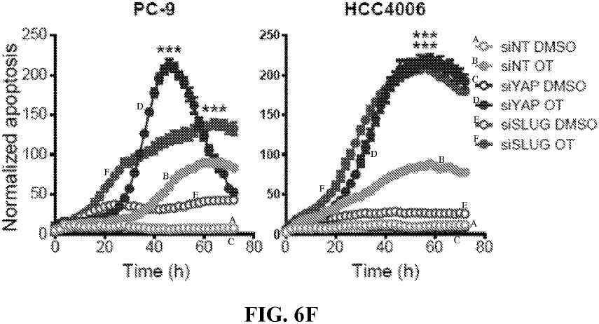

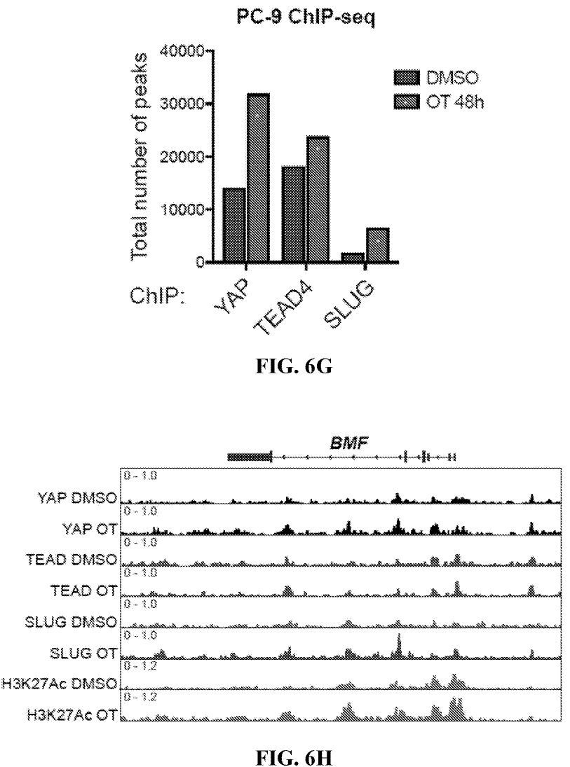

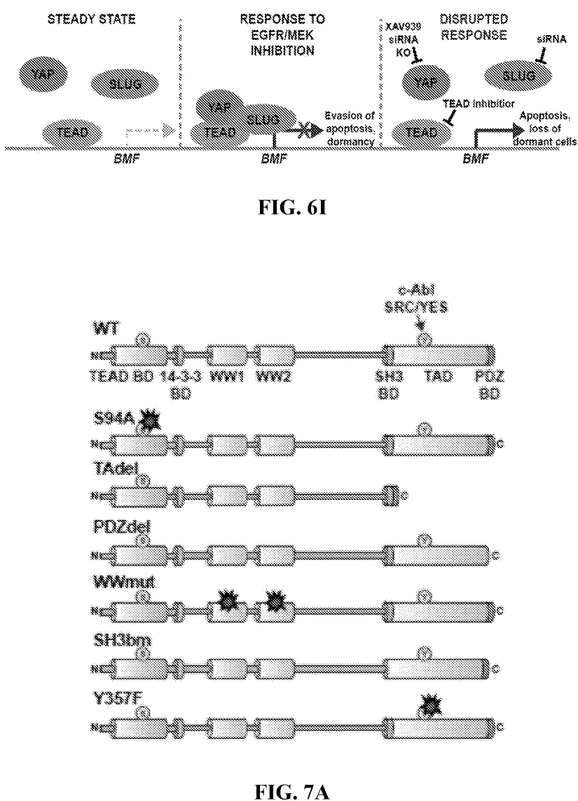

In search for such transcriptional repressors, it was noted that an EMT gene expression signature was highly enriched in the dormant cells induced by osimertinib/trametinib treatment (FIG. 1G). Intriguingly, YAP has been reported to be a mediator of EMT and directly binds to several canonical EMT transcription factors, including SNAIL, SLUG and ZEB1 (Lehmann et al., 2016; Tang et al., 2016). Furthermore, EMT is a known mechanism of drug resistance including EGFR mutant lung cancers (Byers et al., 2013; Sequist et al., 2011; Shibue and Weinberg, 2017; Zhang et al., 2012). It was then explored whether the process of EMT, the development of a dormant state, and the requirement for YAP in mediating evasion of apoptosis through repression of BMF following drug treatment were in fact all linked. In PC-9 and HCC4006 YAP1 KO cells following 24-hour osimertinib/trametinib treatment, compared to control cells, the EMT signature was negatively enriched, suggesting that YAP is triggering the EMT program in EGFR-mutant NSCLC cells (FIG. 6A). QPCR analysis of several EGFR-mutant NCSLC cell lines revealed that SNAI2, encoding the SLUG protein, was the dominantly expressed EMT transcription factor in most cell lines (FIG. 6B). SLUG was focused on as a potential YAP interaction partner in this context. By co-immunoprecipitation, it was shown that endogenous YAP, TEAD and SLUG proteins form a complex in both PC-9 and HCC4006 cells upon 48-hour treatment with osimertinib/trametinib (FIG. 6C). Knock-down of SNAI2 by siRNA in PC-9 and HCC4006 cells resulted in significant increase in BMF expression following 24-hour treatment with osimertinib/trametinib, similar to that observed following YAP1 knock-down (FIG. 6E), and the increase in BMF expression translated into robust increase in apoptosis upon treatment in both cell lines (FIG. 6F). These results suggest that members of the YAP/TEAD/SLUG complex indeed co-operate to repress BMF expression and thus suppress apoptosis in response to osimertinib/trametinib treatment. To confirm that the YAP/TEAD/SLUG complex directly binds the BMF locus to mediate repression, chromatin immunoprecipitation was performed followed by next-generation sequencing (ChIP-seq) using antibodies against endogenous YAP, TEAD4, and SLUG in PC-9 cells treated either with DMSO or with osimertinib/trametinib for 48 hours. Consistent with the treatment-induced increase in YAP activity and subsequent activation of the EMT program, a robust increase in YAP and SLUG binding to chromatin was detected after 48 hours of osimertinib/trametinib treatment, while the TEAD4 chromatin binding was less affected (FIG. 6G). Specifically, overlapping YAP, TEAD and SLUG peaks at the BMF promoter region was observed as well as at nearby H3K27Ac-positive enhancer regions upon treatment (FIG. 6H), demonstrating that the YAP/TEAD/SLUG repressor complex directly binds to BMF locus. Taken together, these results provide a mechanistic explanation for YAP-mediated suppression of pro-apoptotic signaling through the engagement of TEAD and the EMT program to directly repress the induction of BMF expression upon combined EGFR/MEK inhibition (FIG. 6I).

### Development of Novel Covalent TEAD Inhibitor to Target YAP Dependency Upon Combined EGFR/MEK Inhibition

The strict dependency of osimertinib/trametinib-treated cells for YAP presents an attractive target for drug development. While results pointed towards TEAD as the main mediator of YAP effects in this context (FIGS. 2C-2D and FIG. 6H), further confirmation was needed to determine whether other effector pathways downstream of YAP might also play a role in YAP-mediated evasion of apoptosis following osimertinib/trametinib-treatment. The YAP protein has several functional domains, many of which mediate protein-protein interactions (Piccolo et al., 2014). The key functional domains were systematically mutated in YAP (FIG. 7A), and determined which of the mutants could rescue the apoptotic phenotype imparted by YAP1 -deficiency following osimertinib/trametinib treatment in PC-9 cells. Introduction of YAP1 with a mutation in the TEAD-binding domain (S94A) (Zhao et al., 2008), or with a deleted transactivation domain (TAdel; FIG. 7A), had the least ability to rescue YAPl-deficiency (FIG. 7B), confirming that YAP-mediated evasion of apoptosis is absolutely dependent on TEAD, as well as on intact transactivation domain.

As a transcription factor, TEAD has been regarded as an undruggable target. However, recent studies revealed a hydrophobic pocket for the post-translational palmitoylation of TEAD (Chan et al., 2016; Noland et al., 2016), and flufenamic acid as a molecule binding to this pocket (Pobbati et al., 2015). Flufenamic acid was subsequently co-crystallized with TEAD2, demonstrating extensive hydrophobic interactions as its main binding mode (Pobbati et al., 2015). This provided a structural basis for the rational design of a covalent inhibitor for TEAD through an acrylamide as covalent warhead to react with the conserved cysteine 380 on TEAD2. The trifluoromethylated phenyl ring forms extensive hydrophobic interactions, whereas the carboxylic acid of flufenamic acid, proximal to cysteine 380, might be replaced by acrylamide warhead to react with the cysteine. Hence, MYF-01-37 (FIG. 7C) was developed through an extensive chemistry optimization as a covalent binder to TEAD, and targeted cysteine 380 when incubated with TEAD2 protein (C359 in TEAD1) (FIGS. 15A-15C). Pretreating cells with MYF-01-37 led to loss of direct TEAD pull-down by biotin-MYF-01-037 (FIG. 15D) from whole-cell lysate (FIG. 15E), indicating that MYF-01-037 indeed occupied the TEAD pocket in cells. This target engagement of TEAD resulted in inhibition of direct YAP/TEAD interaction (FIG. 7D) in HEK 293T cells, as well as in the reduction in canonical YAP target gene CTGF expression in PC-9 cells (FIG. 7E). This reduction was abrogated by the overexpression of TEAD1 C359S mutant that disrupts the covalent binding of the drug to TEAD, but not by overexpression of wild-type TEAD1 (FIG. 7E), demonstrating that the observed inhibition of YAP activity is due to on-target covalent binding of the compound to TEAD. Importantly, XAV939, which inhibits YAP activity via TEAD-independent mechanism (Wang et al., 2015), was still able to inhibit CTGF expression also in cells expressing the TEAD1 C359S mutant (FIG. 7E). As a single agent, MYF-01-37 compound had minimal impact on cell viability of several EGFR-mutant NCSLC cell lines (FIG. 15F), which is consistent with the apparent dispensability of YAP activity in EGFR-mutant NSCLC cells at steady state (FIG. 2I). When combined with osimertinib/trametinib, MYF-01-37 completely suppressed the increased YAP activity induced by osimertinib/trametinib treatment in PC-9 YAP reporter cells (FIG. 7F), led to a robust increase in BMF expression (FIG. 7G), and subsequently to significantly increased apoptosis in PC-9 and HCC4006 cells compared to osimertinib/trametinib alone (FIG. 7H), thus phenocopying the effects of tankyrase inhibition or YAP1 KO (FIGS. 3A, 3E, 3G and 5D). Importantly, a 10-day treatment with MYF-01-37 in combination with osimertinib/trametinib led to a dramatic decrease in dormant cells compared to osimertinib/trametinib alone in PC-9 and HCC4006 cells (FIG. 7I).

### Discussion

Genotype directed therapy is the standard of care for many cancers that harbor an activated oncogene (Blanke et al., 2008; Drilon et al., 2018; Peters et al., 2017). While this treatment approach has transformed cancer care for many genomic subtypes of cancer, these therapies are rarely, if ever, curative. One explanation for such observations is the inability of genotype directed therapies to eradicate all tumors cells. In EGFR-mutant NSCLC, a complete response is observed only in a small minority (<5%) of patients following treatment with EGFR TKI (Mok et al., 2017; Soria et al., 2018). As EGFR mutations are truncal mutations (i.e. in every cell of a tumor) (Jamal-Hanjani et al., 2017), it is not clear why a proportion of tumor cells can survive initial EGFR inhibitor -induced apoptosis and subsequently persist in the presence of drug treatment.

The development of EMT, as a drug resistant state following EGFR inhibitor treatment, has been observed both in model systems and in lung cancer patients (Byers et al., 2013; Sequist et al., 2011; Zhang et al., 2012). In addition, hyperactivation of the Hippo pathway effector YAP has been shown to dampen the efficacy of targeted treatment in several contexts (Zanconato et al., 2016), including the efficacy of EGFR TKI’s in EGFR-mutant NCSLC (Hsu et al., 2016; Ku et al., 2012). However, the mechanistic bases for these observations remain largely unexplored and unknown. In the current study, a mechanistic link for these two different observations was provided and demonstrated that a critical transcription factor mediating EMT, SLUG, and YAP together lead to transcriptional repression of BMF following EGFR/MEK treatment and as such limits the initial drug induced apoptotic effect allowing the formation of a dormant state.

Apoptosis in response to EGFR TKI’s in EGFR-mutant NCSLC is executed by the intrinsic apoptotic pathway and invariably associated with the up-regulation of BIM (Costa et al., 2007; Cragg et al., 2007; Gong et al., 2007). BIM protein levels are suppressed by the MAPK pathway, both transcriptionally and post-transcriptionally (Ley et al., 2005), and thus mechanisms which disconnect EGFR inhibition from ERK½ inhibition, would be expected to block EGFR inhibitor -mediated upregulation of BIM, promoting cell survival (Ercan et al., 2012; Tricker et al., 2015). Osimertinib can also activate YAP thereby allowing drug-induced cell survival through a completely different mechanism (FIGS. 3A, 3I, 3J, and 4F). Thus, single-agent EGFR TKI treatment can lead to both ERK½-reactivation and YAP activation, whereas upon combined EGFR/MEK inhibition, activation of YAP becomes the dominant survival mechanism in EGFR-mutant NSCLC cells (FIGS. 3A, 3I, 3J, 4F, 4H, and 4I). These results suggest that YAP can maintain the viability of EGFR-mutant NSCLC cells in the chronic absence of EGFR- and its downstream signaling. Intriguingly, the ability of YAP to compensate for the loss of dominant oncogene in MAPK-dependent cancers has been previously shown in the context of mutant KRAS-driven NSCLC and pancreatic ductal adenocarcinoma (Kapoor et al., 2014; Shao et al., 2014). In these studies, YAP1 overexpression (Shao et al., 2014) or YAP1 amplification (Kapoor et al., 2014) was able to rescue the loss of KRAS in a MAPK pathway -independent mechanism.

A novel mechanism was uncovered whereby YAP exerts its protective function through direct transcriptional suppression of BMF by forming a repressor complex with TEAD and the EMT transcription factor SLUG, thus directly linking activation of YAP and EMT with the development of the treatment-induced dormant state. Overexpression of YAP and its paralog TAZ has been shown to induce EMT in a TEAD-dependent manner (Lei et al., 2008; Zhang et al., 2009; Zhao et al., 2008). The senescent-like dormant cells, exhibited high YAP/TEAD activity and demonstrated enriched EMT gene expression signature in vitro and in vivo indicating that these cells have undergone EMT (FIGS. 1G and 4C). Furthermore, the enrichment of the EMT signature was attenuated in YAP1 KO cells upon EGFR/MEK inhibition, suggesting deficient EMT response upon loss of YAP1 (FIG. 6A). Considering that the YAP activation seems to be a specific adaptation mechanism in cells that cannot reactivate EGFR downstream signaling, the YAP/TEAD/SLUG interplay repressing BMF may be the immediate response protecting cells undergoing a YAP-dictated global change in cellular state in the chronic absence of EGFR signaling. Analogously, Shao et al. also found that the YAP-mediated bypass of KRAS loss was associated with the acquisition of a mesenchymal state, suggesting that YAP may drive the EMT program as a mechanism to adapt to the loss of oncogene signaling in other cancer contexts as well (Shao et al., 2014). It is also possible that in addition to YAP/TEAD/SLUG, other factors are involved in the long-term survival of EGFR mutant cancer cells treated with osimertinib/trametinib.

The findings also highlight a previously underappreciated role of BMF in the regulation of apoptosis in EGFR-mutant NSCLC. BMF expression has also been shown to be suppressed by the MAPK pathway (Shao and Aplin, 2010, 2012), which is consistent with BMF up-regulation following EGFR/MEK inhibition also in YAP-proficient cells (FIG. 4D). It was further noted that doxycycline-induced overexpression of BMF alone was sufficient to induce rapid apoptosis in EGFR-mutant NSCLC cells (FIG. 14F). While the levels of overexpressed BMF most likely are non-physiological, this observation further emphasizes the role of BMF, in addition to BIM, in promoting apoptosis in EGFR mutant lung cancer. Whether the observations on YAP mediated suppression of apoptosis through transcriptional repression of BMF also extends to other genotype directed cancer therapies remains unknown and will need to be evaluated in future studies.

Interestingly, the dormant state resulting from YAP/TEAD activation in the study shares several characteristics with cellular senescence, albeit being readily reversible upon drug withdrawal. Treatment-induced senescence (TIS) is repeatedly observed in response to DNA-damaging chemotherapeutic agents (Ewald et al., 2010), but less often in the context of targeted therapy (Haferkamp et al., 2013; Wu et al., 2007). In the case of TIS in response to chemotherapy, the senescent state is mostly regarded as irreversible, and has been suggested to be a compensatory means alongside apoptosis to permanently eradicate cancer cells, and thus a favorable outcome of treatment (Nardella et al., 2011). In contrast to these observations, EGFR-mutant NSCLC cells seem to only reversibly adopt the senescence program upon EGFR/MEK inhibition in order to sustain the otherwise lethal drug exposure, and revert back to the normal steady-state upon drug withdrawal. As a consequence, the senescent-like population, at least in this context, can serve as a reservoir of dormant cells that are later, upon appropriate stimulus, or/and acquisition of resistance mutations, capable of re-establishing the tumor and drive clinically observed drug resistance.

The findings from the study have therapeutic implications for the treatment of EGFR mutant lung cancer. By inhibiting both EGFR and MEK, pro-survival signaling of the remaining cells is shunted towards YAP. The strict dependency of EGFR-mutant NSCLC cells for YAP and TEAD creates a unique EGFR/MEK induced vulnerability for YAP/TEAD antagonism. However, there are currently no available therapies to target YAP/TEAD. Using recent structural and biochemical insights, a novel covalent TEAD inhibitor was developed (Noland et al., 2016; Pobbati et al., 2015). The compound, MYF-01-37, was able to inhibit YAP/TEAD interaction and activity in EGFR-mutant NSCLC cells, and when combined with EGFR/MEK inhibition, was able recapitulate the effects of YAP inhibition observed either through tankyrase inhibition or by YAP1 KO (FIGS. 7D-7I). As YAP is widely associated with resistance to cancer therapies, the effect of TEAD inhibition and/or YAP1 KO on other genotype- or TKI combination contexts was tested within the NSCLC space, including in ALK rearranged, MET amplified and EGFR mutant MET amplified models, and observed increased apoptosis following YAP/TEAD co-targeting in most models (FIGS. 16A-16C). These data suggest wide potential for co-targeting YAP/TEAD with genotype directed therapy. In accordance with the observations that YAP1 loss has negligible consequences in EGFR-mutant NSCLC cells at steady state, MYF-01-37 did not demonstrate single-agent toxicity (FIG. 15F). This is in stark contrast to a recently published covalent TEAD inhibitor TED-347 (Bum-Erdene et al., 2019), which demonstrated significant toxicity in EGFR mutant NSCLC cells as a single agent despite sharing a similar core scaffold with MYF-01-37 (FIG. 15F). These findings are most likely due to differences in the covalent warheads between these two compounds - unlike the highly reactive a-chloroketone covalent warhead in TED-347, the acrylamide warhead in MYF-01-37 is more suitable for covalent targeting of proteins in living cells, and thus most likely contributes to the low non-specific toxicity of MYF-01-37 as a single agent (De Cesco et al., 2017; Liu et al., 2013). Further development is needed to optimize the pharmacological properties of MYF-01-37 to enable preclinical testing of the compound using in vivo models of EGFR-mutant NSCLC.

Ultimately, the strategy of co-targeting EGFR, MEK and YAP/TEAD to enhance the initial treatment efficacy in EGFR mutant NSCLC and limit the establishment of the dormant state, will need to be tested in a clinical trial. Although EGFR and MEK inhibitors can be administered together (NCT03392246; Ramalingam et al., 2019) three drug combinations raise the concern for toxicity. However, in many adult organs, YAP is seemingly dispensable for normal homeostasis, suggesting that targeting YAP might be well tolerated (Zanconato et al., 2016). Also, as the findings reveal, the main effect of YAP1 loss is to enhance the initial apoptotic effect of EGFR/MEK inhibition and thus it is possible that a three-drug combination would only be transiently necessary, followed by a two-drug treatment, thus reducing potential toxicity. In support of this approach, identical potency was observed when PC-9 cells were treated for one week with osimertinib/trametinib/XAV939 or MYF-01-37 followed by two weeks of osimertinib/trametinib compared to a three-week continuous osimertinib/trametinib/XAV939 or MYF-01-37 treatment (FIG. 16D). The potential different treatment approaches will need to undergo clinical evaluation to determine both their efficacy and toxicity. By co-targeting both MEK and YAP/TEAD with EGFR, the goal is to enhance the initial treatment efficacy, limit the formation of dormant cells, ultimately improving the outcome of patients with EGFR mutant NSCLC.

### Methods

### Animal Models

Xenograft studies: Female NCr nude mice, 7-weeks old (for PC-9 studies) and female NSG mice, 6-weeks old (HCC4006 and DFCI243) were purchased from Taconic Biosciences, Inc. and The Jackson Laboratory, respectively. Animals were allowed to acclimate for at least 5 days before initiation of the study. All in vivo studies were conducted at Dana-Farber Cancer Institute with the approval of the Institutional Animal Care and Use Committee in an AAALAC accredited vivarium. The cells were harvested, and 5 × 10⁶ cells with 50% Matrigel (Fisher Scientific) were implanted subcutaneously in the right flank of the NCr nude or NSG mice. For efficacy studies, tumors were allowed to establish to 200 ± 50 mm³ in size before randomization into various treatment groups with 8 mice per group. Osimertinib (10 mg/kg once daily) and trametinib (1 mg/kg once daily) were administered orally as a suspension using 0.5% hydroxypropyl methylcellulose (HPMC) or 0.5% HPMC with 0.2% Tween 80 as vehicle, respectively. Control vehicle treated mice received 0.5% HPMC with 0.2% Tween 80 administered orally. Tumor volumes were determined from caliper measurements by using the formula V = (length × width²)/2. Tumor sizes and body weight were measured twice weekly. Mice were treated for 28 days, followed by measuring for re-growth of tumors. For the single-cell RNA-sequencing and immunohistochemical analysis of in vivo MRD tumors, PC-9 cells were implanted as above. When the tumors reached an average of 200 ± 50 mm³, the mice were randomly assigned to receive either vehicle, 10 mg/kg osimertinib, or 10 mg/kg osimertinib and 1 mg/kg trametinib (3 mice / group). The mice were treated orally once daily for 21 days. After treatment, the tumors were harvested and kept on ice in RPMI-1640 (Gibco), 10% FBS, and 1% penicillin/streptomycin (Gibco) until processing for single-cell RNA-sequencing, or formalin fixed for IHC. For the analysis of BMF expression in vivo, 6 mice/cell line were implanted as above. When the tumors reached an average of 350 ± 50 mm³, the mice were randomly assigned to receive either vehicle or 10 mg/kg osimertinib and 1 mg/kg trametinib (3 mice / group). The mice were treated orally once daily for 3 days, and tumors were harvested 3 hours after the final dose. Tumors were snap-frozen and kept in -80° C. until analysis.

Studies using the EGFR^{L858/T790M} mouse model: All breeding, mouse husbandry, and in vivo experiments were performed with the approval of Dana-Farber Cancer Institute Animal Care and Use Committee. Tumors in the EGFR^{L858R/T790M} mice (Zhou et al., 2009) were induced by 5 × 10⁷ pfu adenovirus expressing Cre Recombinase protein (Cat # VVC-U of Iowa-5, University of Iowa adenoviral core) at 6-8 weeks old and monitored by MRI to quantify lung tumor burden before being assigned to various treatment study cohorts. Mice were treated with either osimertinib or in combination with selumetinib and the lung tumors burden were quantified by MRI imaging before and after the drug treatment. Osimertinib was administered as 5 mg/kg once daily through oral gavage and selumetinib was administered twice daily at 50 mg/kg through oral gavage using 0.5% HPMC as vehicle. For efficacy study, the treatment was continued until 4 weeks then withdrawn. The mice were maintained and monitored by MRI for tumors relapse and humanely euthanized at endpoint. For short term study in order to acquire residual tumors samples, mice were euthanized and samples harvested after treating with osimertinib until MRI imaging showed no visible tumor (2 weeks).

### Cell Line Authentication

293T cells and the NSCLC cell lines PC9, HCC827, HCC4006, HCC2279, H1975, H3122, EBC-1 and the patient-derived DFCI243 cell line were grown in RPMI-1640 (Gibco), 10% FBS, and 1% penicillin/streptomycin (Gibco). NCI-H226 cells, 293T cells, and the NSCLC cell lines PC9, HCC827, HCC4006, HCC2279, H1975, H3122, EBC-1 and the patient-derived DFCI243 cell line were grown in RPMI-1640 (Gibco), 10% FBS, and 1% penicillin/streptomycin (Gibco). The HCC827 and HCC2279 cells were obtained from Dr. Adi Gazdar (UT Southwestern, Dallas, TX) in 2004. The PC9 cells were obtained from Dr. Nishio (Kinki University, Osaka, Japan) in 2005. HCC4006, H1975, 293T and EBC-1 were purchased from ATCC. DFCI243 and HCC827 GR6 (Engelman et al., 2007) cell lines were established in the Jänne laboratory. NCI-H226, HCC4006, H1975, 293T and EBC-1 were purchased from ATCC. DFCI243 and HCC827 GR6 (Engelman et al., 2007) cell lines were established in the Jänne laboratory. Cell line identity was confirmed by fingerprinting for the following cell lines: HCC4006, PC9, HCC827, and HCC2279. H1975 and 293T, and EBC-1 cells were purchased from ATCC in 2016, 2017, and 2017, respectively and were not fingerprinted. H1975 and 293T, EBC-1, and NCI-H226 were purchased from ATCC in 2016, 2017, and 2019, respectively and were not fingerprinted.

### Reagents

Osimertinib was purchased from MedChem Express, trametinib, ZSTK474, AZD2014, ruxolitinib, XAV939, crizotinib, gefitinib, and cisplatin were purchased from Selleck Chemicals, NVP-BEZ235, galuniserib, sotrasaurin, saracatinib, and BMS-345541 were purchased from Cayman Chemicals. All drugs were prepared to 5 mM to 10 mM stock solutions in DMSO and stored in -80°e. 100 nM osimertinib and 30 nM trametinib were used in all assays, other compounds were used in concentrations indicated in the figures or the figure legends.

### Expression Vectors

All YAP1 constructs used in this study harbor the cDNA encoding 488 amino acid YAP1 isoform (Sudol, 2012). Wild-type YAP1 and YAP1-WWmut cDNAs were amplified from p2xFlagCMV2-YAP2 and p2xFlagCMV2-YAP2-1st&2nd WW mutant plasmids (gifts from Marius Sudol, Addgene plasmids #19045 and #19048, respectively) and subcloned into pDNR-dual (BD Biosciences) using SalIand XbaI restriction sites. pDNR-dual-YAP1-S94A and pDNR-dual-YAP1SH3bm were created by amplifying the mutation sites from pLX304-YAP1(S94A) and pLX304-YAP1 _SH3bm plasmids (gifts from William Hahn, Addgene plasmids #59145 and #59141, respectively) using primers 5′-ATCAACGGGACTTTCCAAAATGTCG-3′ (SEQ ID NO: 1) and 5′-TTTTTTTCTAGACTATAACCATGTAAGAAAGCTTTCTTTA-3′ (SEQ ID NO: 2) and subcloning the amplified regions into pDNR-Dual-YAP1 using BamHI and XbaI restriction sites. Because the pLX304-YAP1(S94A) and pLX304-YAP1_SH3bm contain the YAP1-504 isoform, a 48 base pair region from pDNR-Dual-YAP1S94A and pDNR-Dual-YAP1SH3bm was subsequently deleted to create the YAP1-488 isoforms. The deletions were done by PCR using primers 5‘-GAGTTAGCCCTGCGTAGCCA-3′ (SEQ ID NO: 3) and 5′-CTGCCGAAGCAGTTCTTGCT-3′ (SEQ ID NO: 4) followed by re-ligation of the PCR product. The PDZ-deletion mutant of YAP1 was created by PCR from p2xFlagCMV2-YAP2 using primers 5‘- TTTTTTGTCGACCAGAATTGATCTACCATGGACT-3′ (SEQ ID NO: 5) and 5‘-TTTTTTTCTAGACTAGCTTTCTTTATCTAGCTTGGTG-3′ (SEQ ID NO: 6) and subcloning the PCR product into pDNR-dual using SalIand XbaI restriction sites. The YAP1-TAdel cDNA was amplified from pLX304-YAP1_TA (gift from William Hahn, Addgene plasmid #59143) using primers 5′- ATCAACGGGACTTTCCAAAATGTCG-3′ (SEQ ID NO: 1) and 5′-TTTTTTTCTAGACTATAACCATGTAAGAAAGCTTTCTGGGCT-3′ (SEQ ID NO: 7) and subcloned into pDNR-dual-YAP1 using BamHI and XbaI restriction sites. All YAP1 cDNAs were subsequently shuttled into JP1722 expression vector using the BD Creator System (BD Biosciences).

The TEAD1 C359S mutation was generated into pRK5-myc-TEAD1 backbone (a gift from Kunliang Guan, Addgene plasmid #33109) by PCR using primers 5′-TCCCCAATGAGTGAATATATGATCAAC-3′(SEQ ID NO: 8) and 5′-GCGGTTTATTCGGTATACAAATCG-3′ (SEQ ID NO: 9). Both wild-type myc-TEAD1 and the myc-TEAD1 C359S mutant cDNAs were amplified from the pRK5-backbone using primers 5‘-GGGGACAAGTTTGTACAAAAAAGCAGGCTTCGCCACCATGGAGCAAAAGCT CATCTCAG-3′ (SEQ ID NO: 10) and 5′-GGGGACCACTTTGTACAAGAAAGCTGGGT CAGTCCTTTACAAGCCTGTAAATATG-3′ (SEQ ID NO: 11) and shuttled into the pLEX307 lentiviral vector (a gift from David Root, Addgene plasmid #41392) using the Gateway cloning technology (Invitrogen). The TBS-mCherry vector has been described previously (Mohseni et al., 2014).

### Cell Growth and Viability Assays

For FIGS. 1A and 1C, 350 cells/well were plated into 96-well plates and treated as indicated in the n(n=60 wells / condition). Medium with fresh drugs was changed every 3-5 days. The confluency of the wells was determined weekly using the Incucyte FLR live cell analysis system (Essen Bioscience). For FIG. 8B, cells were plated and treated as above, and the wells were manually scored as positive when the confluence was above 50 % and assessed weekly (Tricker et al., 2015). For FIG. 8C, 78 000 PC-9 cells were plated into T25 flasks and treated the next day as indicated. Cell proliferation was monitored using Incucyte HD live cell analysis system (Essen Bioscience) by imaging 32 sectors in the T25 flask. For all other long-term growth assays (≥10 days), 1000 cells/well were plated into 96-well plates and treated as indicated in the figures (n=5-12 wells / condition). The confluency of the wells was determined daily using Incucyte HD. Endpoint cell viability assays were performed using Cell Titer Glo (Promega) according to manufacturer’s instructions. For FIG. 17, 200 NCI-H226 cells were plated in 384-well plate and treated the next day, and the cell viability were measured on day 5 using Cell Titer Glo (Promega) according to the manufacturer’s instructions.

To determine number of dormant cells after treatment, viable cells were manually counted from the Incucyte images. A total of 10-12 wells with 3 images per well was analyzed for each condition.

### Western Blotting and Antibodies

If not specified below, cells were plated at 15 × 10⁴ cells / cm², treated the next day (if applicable) and lysed at specified timepoints in RIPA buffer (Boston Bioproducts) supplemented with cOmplete Mini EDTA-free Protease inhibitor cocktail (Roche) and PhoSTOP phosphatase inhibitor cocktail (Roche). Twenty micrograms of total protein was used for immunoblotting according to the antibody manufacturer’s recommendations. For the assessment protein levels in dormant cells, 13 × 10⁴ cells / cm² were plated into 2 × 15 cm dishes. Cells were treated the next day and medium with fresh drugs was changed every 3-5 days. Cells were trypsinized at specified timepoints, washed with ice-cold PBS, and the cell pellets were lysed and immunoblotted as above.

The following antibodies were purchased from Cell Signaling: phospho-EGFR (#3777), EGFR (#2232), phospho-AKT (#4058), AKT (#9272), phospho-ERK (#4370), ERK (#9102), phospho-S6 (#2215), S6 (#2217), YAP (#14074), BIM (#2933), BCL-XL (#2764), BCL-2 (#4223), BCL-w (#2724), MCL-1 (#39224), p27^{Kip1} (#3686), p21^{Cip1} (#2947), p16^{INK4A} (#80772), pan-TEAD (#13295), BAX (#5023), SLUG (#9585), HA (#2367). Anti a-tubulin antibody (T9026) was purchased from Sigma-Aldrich. ApoTrack™ Cytochrome c Apoptosis WB Antibody Cocktail was purchased from Abcam (ab110415). HSP90 antibody (sc-7947) was purchased from Santa Cruz Biotechnology.

### Cellular Barcoding

PC-9 cells were transduced with the EvoSeq barcode library (Feldman et al., 2019) and bottlenecked to a complexity of approximately 500 000 barcodes. The barcoded cells were plated into five replicates / treatment, 5x10⁶ cells / replicate. The cells were then treated with 300 nM gefitinib, 100 nM osimertinib, or 100 nM osimertinib + 30 nM trametinib for 3 weeks to establish the residual cell populations. After treatment, the cells were harvested and the genomic DNA was extracted, the barcode-containing sequences were amplified from the genomic DNA, and prepared for sequencing as described (Feldman et al., 2019). Each library was quantified by Qubit fluorometer, Agilent TapeStation 2200, and RT-qPCR using the Roche Kapa Biosystems library quantification kit according to manufacturer’s protocols. Uniquely indexed libraries were multiplexed in equimolar ratios into two pools - one pool of twelve libraries and the other of thirteen libraries - and sequenced on two Illumina NextSeq500 runs with paired-end 75 bp reads by the Dana-Farber Cancer Institute Molecular Biology Core Facilities. The downstream analyses were done as described previously (Bhang et al., 2015).

### RNA Extraction and Quantitative PCR (QPCR)

Cells were plated at 15 × 10⁴ cells / cm², treated the next day, and RNA samples were extracted at specified timepoints using the RNeasy Mini kit (Qiagen). The RNA concentrations were measured with Nanodrop (Thermo Fisher Scientific) and 1 µg of total RNA was used for cDNA synthesis using the QuantiTect Reverse Transcription Kit (Qiagen). The QPCR reactions were set up in 20 µl using Taqman Gene Expression Master Mix (Applied Biosystems, cat. 4369016), Taqman Gene Expression Assays (Applied Biosystems) as per manufacturer’s instructions, and 2 µl of 1:10 diluted cDNA. The following Taqman Gene Expression Assays were used in the study: CTGF (Hs01026927_ml), ANKDR1 (Hs00173317_ml), BMF (Hs00372937 _m1), SNAI1(Hs00195591_m1), SNAI2 (Hs00161904_m1), TWIST1 (Hs00361186_m1), TWIST2 (Hs02379973_s1), ZEB1 (Hs00232783_m1), ZEB2 (Hs00207691_m1), and ACTB (Hs01060665_g1). The reactions were run in StepOne Plus Real-time PCR System (Applied Biosystems) using default reaction settings. The gene expression levels were normalized to ACTB housekeeping gene expression levels in each sample.

For the analysis of BMF expression in vivo, RNA was extracted from 25-30 mg snap-frozen tumor samples using RNeasy Mini kit according to the kit protocol. Reverse transcription and gene expression analyses were performed as above.

### RNA-Sequencing

To analyze gene expression changes associated with dormancy, PC-9, HCC827 and HCC4006 cells were plated at 15 × 10⁴ cells / cm² into 10 cm plates (DMSO treated control cells) or into 15 cm plates (dormant cells). The next day, cells were treated either with DMSO or with the combination of 100 nM osimertinib and 30 nM trametinib in duplicate. DMSO-treated control cells were harvested 24 h later, and the dormant cells after 2 weeks of treatment. At these timepoints, cells were lysed into TRIzol and RNA extraction was performed according to the manufacturer’s protocol.

In order to analyze YAP1 KO -associated gene expression changes, PC-9 and HCC4006 CTRL and YAP1 KO cells were plated into 10 cm dishes at 15 × 10⁴ cells / cm². The next day, the cells were treated with DMSO or with the combination of 100 nM osimertinib and 30 nM trametinib in triplicate. After 24 hours, the cells were lysed into TRIzol and RNA extraction was performed according to the manufacturer’s protocol.

Libraries were prepared using Illumina TruSeq Stranded mRNA sample preparation kits from 500 ng of purified total RNA according to the manufacturer’s protocol. The finished dsDNA libraries were quantified by Qubit fluorometer, Agilent TapeStation 2200, and RT-qPCR using the Kapa Biosystems library quantification kit according to manufacturer’s protocols. Uniquely indexed libraries were pooled in equimolar ratios and sequenced on an Illumina NextSeq500 with single-end 75 bp reads by the Dana-Farber Cancer Institute Molecular Biology Core Facilities. Sequenced reads were aligned to the UCSC hg19 reference genome assembly and gene counts were quantified using STAR (v2.5.1b). Differential gene expression testing was performed by DESeq2 (v1.10.1) and normalized read counts (FPKM) were calculated using cufflinks (v2.2.1). RNAseq analysis was performed using the VIPER snakemake pipeline (Cornwell et al., 2018).

Gene set enrichment analyses from the RNA-seq data were performed according to the instructions (broadinstitute.org/gsea/index.jsp).

### Senescence-Associated β-Galactosidase Staining

PC-9, HCC827 and HCC4006 were plated into 6-well plates at 50 000 cells / well, and treated the next day with DMSO, 100 nM osimertinib or with the combination of 100 nM osimertinib and 30 nM trametinib in triplicate. DMSO-treated control cells were stained after 72 h, and osimertinib and osimertinib/trametinib treated cells were stained after 10-day treatment using Senescence β-Galactosidase Staining Kit (Cell signaling #9860) according to manufacturer’s protocol. After staining, cells were imaged (5 images / well), and stained cells were manually counted from the images.

### Cytokine Profiling

Multiplex assay was performed using the Human Cytokine/Chemokine Magnetic Bead Panel (Millipore cat# HCYTMAG-60K-PX30) on a Luminex MAGPIX system (Millipore). Conditioned media concentration levels of each protein were derived from 5-parameter curve fitting models. Protein levels were normalized to cell number in each well.

### Immunofluorescence Staining and Imaging

Cells grown on coverslips were washed with PBS and fixed with 4 % PFA for 10 minutes. The cells were then permeabilized with 0.1 % Triton-X-PBS, followed by a blocking step in 1 % BSA-PBS. The cells were incubated for 60 minutes with Anti-Histone H3 (tri methyl K9) antibody (ab8898, Abcam) (FIG. 1K) or with anti-YAP (Cell Signaling #14074) (FIG. 3B), washed 3 times with PBS, incubated with Alexa Fluor 488® -conjugated secondary antibody (A-11008, Life Technologies) for 45 minutes, and washed 3 times with PBS. The nuclei were counterstained with 1 µg/ml DAPI (Cell Signaling #4083). The coverslips were mounted using Immu-Mount reagent (Thermo Scientific). Images were acquired using Leica SP5 X confocal microscope (Confocal and Light Microscopy Core, DFCI). Image analysis was performed using ImageJ software. For Anti-Histone H3 (tri methyl K9), Images were segmented using standard thresholding parameters and objects were automatically counted using ImageJ Analyze particles- plugin. For the analysis of YAP nuclear localization, the Intensity Ratio Nuclei Cytoplasm Tool -plugin was used.

### ATAC-Sequencing

PC-9 cells were plated at 15 × 10⁴ cells/cm² into 15 cm plates, and treated the next day with DMSO, 100 nM osimertinib or with the combination of 100 nM osimertinib and 30 nM trametinib in triplicate. DMSO-treated control cells were harvested 24 h later. Osimertinib and osimertinib/trametinib treated cells were harvested after 2 weeks of treatment. Rebound samples were obtained by withdrawing drugs from three additional osimertinib/trametinib-treated plates and harvesting the cells once the plates reached 60-70% confluence. Cells were trypsinized at timepoints, and cryopreserved in FBS + 8% DMSO in -80C until processing. After all samples were harvested, 50000 cells / sample were resuspended in 1 ml of cold ATAC-seq resuspension buffer (RSB; 10 mM Tris-HCl pH 7.4, 10 mM NaCl, and 3 mM MgC12 in water). Cells were centrifuged at max speed for 5 min in a pre-chilled (4 C) fixed-angle centrifuge. After centrifugation supernatant was carefully aspirated. Cell pellets were then resuspended in 50 µl of ATAC-seq RSB containing 0.1% NP40, 0.1% Tween-20, and 0.01% digitonin by pipetting up and down three times. This cell lysis reaction was incubated on ice for 3 min. After lysis, 1 ml of ATAC-seq RSB containing 0.1% Tween-20 (without NP40 or digitonin) was added, and the tubes were inverted to mix. Nuclei were then centrifuged for 5 min at max speed in a pre-chilled (4 C) fixed-angle centrifuge. Supernatant was removed and nuclei were resuspended in 50 µl of transposition mix (Corces et al., 2017): 2.5 µl transposase (. final), 16.5 µl PBS, 0.5 µl 1% digitonin, 0.5 µl10% Tween-20, and 5 µl water) by pipetting up and down six times. Transposition reactions were incubated at 37 C for 30 min in a thermomixer with shaking at 1,000 r.p.m. Reactions were cleaned up with Qiagen columns. Libraries were amplified as described previously (Buenrostro et al., 2015). 36-bp paired-end reads were sequenced on a Nextseq instrument (Illumina).

### ChIP-Sequencing

PC-9 cells were plated at 15 × 10⁴ cells / cm² into 15 cm plates, and treated the next day either with DMSO or with the combination of 100 nM osimertinib and 30 nM trametinib in duplicate. Cells were trypsinized at after 48 h of treamtent, and cryopreserved in FBS + 8% DMSO in -80C until processing. Cells were washed in phosphate-buffered saline (PBS) and crosslinked with 1% Formaldehyde for 10 minutes (H3K27Ac) or crosslinked with two agents starting with 2 mM DSG (Pierce) for 45 min at RT, followed by 1 ml 1% Formaldehyde for 10 min (YAP and TEAD4). Crosslinked Cell lines were quenched with 0.125 M glycine for 5 min at room temperature. After quenching, the material was resuspended in 1% SDS (50 mM Tris-HCl pH8, 10 mM EDTA) and sonicated for 5 minutes with a Covaris E220 instrument, 5% duty cycle, 140 Peak Incident Power, 200 Cycles per burst, in 1ml AFA Fiber milliTUBEs. Soluble chromatin was immunoprecipitated with 10 µg of H3K27ac antibody (Diagenode cat# C15410196 lot#A1723-0041D), 7 µg of YAP antibody (Cell signaling #14074), or 1.5 µg of TEAD antibody (ab58310, Abcam). 5 µg of chromatin was used for H3K27Ac ChIP, and 40 µg for YAP, TEAD4 and SLUG ChIPs. ChIP-seq libraries were constructed using Accel-NGS 2S DNA library kit from Swift Biosciences. Fragments of the desired size were enriched using AMPure XP beads (Beckman Coulter). 36-bp paired-end reads were sequenced on a Nextseq instrument (Illumina).

### ATAC-Seq and ChIP-Seq Analyses

The raw data from ATAC-seq and ChIP-seq was first ran through the ChiLin 2.0.0. pipeline (Qin et al., 2016) for initial quality control and preprocessing. Reads were mapped to human genome (hg19) using Burrows-Wheeler Aligner (Li and Durbin, 2010) and peak calling was performed using MACS2 (Zhang et al., 2008b). The output bedgraph files from MACS2 were converted to bigwig files for visualization in the Integrative Genomics Viewer. Deeptools (Ramirez et al., 2016) was used to create heatmap plots. The PCA plot was generated by using the R package ‘ggbiplot’. HOMER (Heinz et al., 2010) was used for the motif analysis.

### CRISPR/CAS9 Gene Editing

YAP1 knock-outs were performed by CRISPR/CAS9 genome editing using the Alt-R CRISPR-CAS9 system (Integrated DNA Technologies, IDT) and Lonza 4D-Nucleofector (Lonza), following previously described protocol (Richardson et al., 2016). Guide sequences for YAP1 were designed using Deskgen (deskgen.com), and the corresponding Alt-R CRISPR-Cas9 crRNAs (crRNA) were ordered from IDT. The crRNA was hybridized with Alt-R CRISPR-Cas9 tracrRNA (tracrRNA, IDT) by mixing 120 pmol of crRNA with 120 pmol of tracrRNA in 5 µl of CAS9 buffer (20 mM HEPES (pH 7.5), 150 mM KCl, 1 mM MgCl2, 10% glycerol and 1 mM TCEP), incubating the mixture at 95° C. for 5 minutes and then letting the mixture cool to room temperature on benchtop (5-10 minutes). 100 pmol of Alt-R® S.p. Cas9 Nuclease V3 (IDT) in 5 µl of CAS9 buffer was slowly added to the crRNA:tracrRNA duplex and the subsequent solution was incubated for 20 minutes in room temperature to allow ribonucleoprotein complex (RNP) formation. The RNP complex was then added to 20 µl of cell suspension containing 300 000 cells suspended in Nucleofector SE Cell line solution (Lonza, cat. V4XC-1032), mixed and 20 µl of the cell/RNP mix was pipetted into one well of a Nucleocuvette Strip (Lonza, cat. V4XC-1032). The reaction mixtures were nucleofected using cell line -specific programs (see below) in the 4D-Nucleofector, and finally transferred to 6-well plates. After 72 hours, the nucleofected cells were single-cell cloned, and loss of YAP protein expression was analyzed from the single-cell clones by western blotting. The guide sequence 5′- TAATAGGCCAGTACTGATGC-3′ (SEQ ID NO: 12) was used to create PC-9, HCC4006, and DFCI243 YAP1 KOs. H3122 and EBC-1 YAP1 KOs were created using two guides with sequences 5′-TAATAGGCCAGTACTGATGC-3′ (SEQ ID NO: 12) and 5′-GAATGAGCTCGAACATGCTG-3′ (SEQ ID NO: 13) simultaneously to ensure high knockout efficiency. The nucleofected H3122 and EBC-1 cells were not single-cell cloned, and bulk populations were used in the experiments. The nucleofection conditions were optimized using the Cell Line Optimization 4D-Nucleofector X Kit (Lonza, cat. V4XC-9064) following the kit protocol. The optimized programs used were: EN-138 for PC-9 and EBC-1 cells; CA-137 for H3122 cells; CM-137 for HCC4006 and DFCI243. All cell lines were nucleofected in SE Cell line solution.

To tag BMF gene with a N-terminal HA-tag in the endogenous locus, PC-9 cells were nucleofected as above in the presence of 150 pmol of single-stranded donor oligonucleotide 5′GCTGAGGGGGCAGTCCAGTAGGCTCTGGGCAAACAGGTCAGCAGAGAGCAAG CTCCCGGGTTGGGTCACCGGCTCCCCATCCTCTGGTTGGAACACATCATCCTCCA GCTCCTCCACACACTGAGATGGCTCAGCGTAATCTGGTACGTCGTATGGGTACAT CTCTCCTGTGAGGGGGCAACGCAGGCATCTGGGCTGCT-3′ (SEQ ID NO: 14) (Ultramer®, IDT). Single-cell clones were screened for donor integration by PCR using primers 5′- AGAAGGGAAGGGGAGTCCTT-3′ (SEQ ID NO: 15) and 5′-CGTAATCTGGTACGTCGT ATGGGTA-3′ (SEQ ID NO: 16), and positive clones were verified by Sanger sequencing.

### Monitoring Caspase-3/7 Activity

Cells were plated into 96-well plates at 3000 cells/well in 100 µl of growth medium. The next day, drugs were added onto cells in 50 µl containing CellEvent™ Caspase-3/7 Green ReadyProbes™ Reagent (Molecular Probes) as per manufacturer’s instructions (n=5-6 wells / condition). The wells were subsequently scanned every 2 hours using the Incucyte ZOOM live cell analysis system (Essen Bioscience) typically for a total of 72 hours. The acquired fluorescent signal for activated caspase-3/7 was normalized with well confluency at each timepoint (=normalized apoptosis). Peak apoptosis was determined as the highest normalized caspase-3/7 activity value during the assay.

### Determining YAP Activity and Apoptosis in PC-9 YAP/Hippo Reporter Cells

3000 cells / well were plated into 96-well plates and treated the next day with the indicated drugs (n=5-6 wells / condition). YAP activity -induced mCherry expression was quantified using the Incucyte ZOOM live-cell analysis system. The mCherry signal was normalized to well confluency at each time point. For simultaneous detection of YAP activity and apoptosis, the cells were plated as above, and treated in the presence of CellEvent™ Caspase-3/7 Green ReadyProbes™ Reagent (Molecular Probes) as per manufacturer’s instructions. The mCherry signal as well as the green fluorescence signal was quantified every 2 hours using Incucyte ZOOM.

To determine the odds ratio for YAP^{high} cells undergoing apoptosis in response to osimertinib/trametinib treatment, the number of YAP^{high} cells (cells with higher mCherry signal than untreated cells), apoptotic cells (positive for green fluorescence) and apoptotic YAP^{high} cells (YAP^{high} cells positive for green fluorescence) was determined using the Incucyte ZOOM software. The analyses were done at a single timepoint corresponding to the peak in apoptosis in response osimertinib/trametinib treatment (72-80 hours after the start of treatment, depending on the experiment), and 5-6 wells with 3 images / well were analyzed. From the same images, the total number of cells per image was manually determined. Using these metrics, a contingency table was built for the average number of YAP^{high} caspase-3/7 positive, YAP^{high} caspase-3/7 negative, YAP^{low}, caspase-3/7 positive, and YAP^{low}, caspase-3/7 negative cells. The odds ratio was computed in GraphPad Prism 7.04 software, and two-sided Fisher’s exact test was used to analyze statistical significance.

The proportion of YAP^{high} dormant cells (FIG. 3I) was determined manually from the Incucyte images after 10-day treatment. 5-6 wells with 3 images per well were analyzed.

### Viral Transductions

For stable expression of YAP1 or YAP1 mutants, PC-9 YAP1 KO and HCC4006 YAP1 KO cells were transduced with lentivirus according to previously described standard protocol (Bahcall et al., 2016). Transduced cells were selected with 2 µg/ml puromycin. PC-9 YAP/Hippo reporter cells were created by lentiviral transduction of TBS-mCherry YAP/Hippo reporter construct (Mohseni et al., 2014). The subsequent cell pool was flow sorted for EGFP expression to select transduced cells.

### Single-Cell RNA Sequencing

For in vitro samples: PC-9 cells were plated onto T75 flasks at 1.5 × 10⁶. Cells were treated either with DMSO or with 100 nM osimertinib and 30 nM trametinib for three weeks. After treatment, the cells were washed with PBS, trypsinized, and loaded onto a 10X Chromium instrument (10X Genomics) per the manufacturer’s instructions. For in vivo samples: Fresh tumor specimens were pooled and minced in a 15 ml conical tube with media (DMEM + 10% FBS), penicillin-streptomycin (Fisher Scientific), 100 U/mL collagenase type IV (Life Technologies) and 2.5 mg/mL DNAse I (Sigma Aldrich), then incubated for 45 min at 37° C. Single cell suspensions were isolated by straining through a 40 µm filters. Cells were incubated with Zombie Green™ Fixable Viability Kit (BioLegend), blocked with Human TruStain FcX™ (BioLegend), and stained with human anti-EpCAM (clone 9C4). Viable EpCAM+ tumor cells were isolated via FACS Melody instrument (BD Biosciences) according to gating schema (FIG. 13A). Cells were loaded onto a 10X Chromium instrument (10X Genomics) per the manufacturer’s instructions.

Single-cell RNA libraries were generated using the Single Cell 3′ Reagent Kit (10X Genomics) per user guide. Quality control of the completed libraries was performed using Bioanalyzer High Sensitivity DNA Kit (Agilent) and then sequenced using the Illumina NextSeq 500 platform by Novogene. The single-cell RNA-Seq data were processed with CellRanger software package (v.3.0.2). Briefly, the bcl files were converted to fastq files, which were aligned to human transcriptome (build GRCh38). After initial filtering with default parameters, the feature matrix generated by Cell Ranger was used to perform downstream analysis using R toolkit Seurat (v.3.0) (Butler et al., 2018). At this step, cells with mitochondria percentages greater than 20 or expressing less than 200 genes were filtered out. Raw counts were normalized using LogNormalize approach with scaling factor set to 10,000. Clustering was performed using Uniform Manifold Approximation and Projection method (UMAP) (Becht et al., 2019).

To characterize cell subpopulations in the samples, the gene signature enrichment analysis was performed for “YAP signature”, “HALLMARK EMT signature” (HALLMARK_EPITHELIAL_MESENCHYMAL_TRANSITI

NSITION, MSigDB, (software.broadinstitute.org/gsea/msigdb/index.jsp), and “FRIDMAN SENESCENCE UP” (MSigDB) signatures. The YAP signature was curated from gene sets obtained from multiple studies (Cordenonsi et al., 2011; Dupont et al., 2011; Wang et al., 2018; Zhang et al., 2009, 2008a). The YAP signature was filtered to include only those genes that were associated with strong YAP binding upon osimertinib/trametinib treatment based on the ChIP-Seq data (peaks with ChIP/input enrichment fold-change greater than 10). Using the single-cell RNA-Seq data, the enrichment scores for a given gene signature for each cell in a sample with R package AUCell was calculated (Aibar et al., 2017).

### Immunohistochemistry

Five-micron paraffin sections were stained on a Leica BondRXⓇ autostainer, according to the manufacturers’ instructions, with primary antibodies against F480 (Cell Signaling, cat # 70076S, 1:500), CD4 (Cell Signaling; cat # 25229; 1:100), CD8a (Cell Signaling; cat # 98941S; 1:400), YAP (Cell Signaling; Cat # 14074, 1:200) and TTF1 (Abcam, AB 133638, 1:50). Prior to antibody incubation, the sections were heat-retrieved with ER1 buffer (pH 6; Leica AR9961) for 20 minutes (YAP), ER2 (pH 9; Leica AR9640) for 20 minutes (CD4, CD8a, F480) or ER2 for 60 mins (TTF1) (Leica, AR9640) at 100° and then treated for 5 minutes with hydrogen peroxide. Sections were incubated with primary antibodies for 30 minutes (CD4, CD8a, F480, YAP) or 60 minutes (TTF1), followed by Leica anti-rabbit HRP-conjugated polymer, and then developed with DAB, counterstained with hematoxylin (Leica DAB KIT, Cat # DS9800) and mounted with permount.

The IHC stainings were quantified using the QuPath software (0.2.0-m4) (qupath.github.io). The Positive Cell Detection -analysis was used with default settings to detect and quantify cells staining positive for pERK, CD4, CD8, TTF1, or the nuclear staining of YAP. Five individual, randomly selected fields per tumor were quantified. Quantified values from each individual field from all tumors are shown in the graphs to represent the heterogeneity in the tumor samples.

### Detection of Activated BAX

For the analysis of BAX activation, cells (15 × 10⁴ cells / cm²) growing on 10 cm plates were treated for 24 hours with DMSO or with the combination of 100 nM osimertinib and 30 nM trametinib, and lysed in CHAPS lysis buffer (50 mM Tris-HCl, 1 % CHAPS, 150 mM NaCl, 5 mM EDTA) supplemented with protease and phosphatase inhibitors. 1000 µg of total protein was used for immunoprecipitation with 1 µg of conformation-specific BAX antibody (clone 6A7; MA5-14003, Invitrogen) and 20 µl of Protein A/G PLUS-Agarose beads (Santa Cruz Biotechnology). The lysates were incubated with the antibody and beads overnight at +4° C., after which the beads were washed four times with 1000 µl of CHAPS buffer, resuspended to 50 µl SDS Sample Buffer (Boston Bioproducts), and incubated at +95° C. for 5 minutes. Activated BAX was detected by immunoblotting using an antibody detecting total BAX (Cell Signaling #5023). For control, total BAX levels were also determined from total cell lysates.

### Detection of Mitochondrial Cytochrome C Release

Cells (15 × 10⁴ cells/cm²) growing on 15 cm plates were treated with the combination of 100 nM osimertinib and 30 nM trametinib for 24 hours, and fractionated into mitochondrial and cytosolic fractions using Cell Fractionation Kit - Standard (ab109719, Abcam) according to the manufacturer’s instructions. Cytochrome c and ATP synthase subunit alpha (mitochondrial marker) was detected from the fractions by immunoblotting using ApoTrack™ Cytochrome c Apoptosis WB Antibody Cocktail (ab110415, Abcam). MEK ½ was used as the cytosolic marker (Cell Signaling #9122).

### Gene Knock-Down by siRNA

Cells were plated on 6-well plates at 15 × 10⁴ cells/cm². The next day, the cells were transfected with 10 nM siRNAs using DharmaFECT 1 (Dharmacon) according to manufacturer’s protocol. Forty-eight hours later, the cells were trypsinized and plated into experiments. The following Dharmacon SMARTpool ON-TARGET siRNA pools were used in the analyses: BMF (L-004393-00-0005), SNAI1 (L-010847-01-0005), SNAI2 (L-017386-00-0005), TWIST1 (L-006434-00-0005), TWIST2 (L-012862-02-0005), ZEB1 (L-006564-01-0005), ZEB2 (L-006914-02-0005), YAP1 (L-012200-00-0005) as well as ON-TARGETplus Non-Targeting Pool (D-001810-10-05). Gene knock-down was controlled by western blotting or QPCR 72 h after transfection.

### Co-Immunoprecipitation

PC-9 and HCC4006 cells treated for 48h with either DMSO or with the combination of 100 nM osimertinib and 30nM trametinib were lysed in IP buffer (1% Triton-X100, 50 mM Tris, 150 mM NaCl, pH 7.4) supplemented with cOmplete Mini, EDTA-free Protease inhibitor cocktail (Roche) and PhoSTOP phosphatase inhibitor cocktail (Roche). 1500 µg of total protein was used for immunoprecipitation with Cell Signaling antibodies recognizing endogenous YAP (#14074), TEAD (pan-TEAD, (#13295), or SLUG (#9585) and 20 µl of Protein A/G PLUS-Agarose beads (Santa Cruz Biotechnology; sc-2003). Immunoprecipitations were carried out overnight at +4° C., followed by four washes with 1 ml IP buffer. After washes, the beads were re-suspended in SDS Sample Buffer (Boston Bioproducts), and boiled for 5 min. Co-immunoprecipitated proteins were analyzed by western blotting.

### YAP/TEAD Interaction Assay

293T cells were plated in 24-well plates and transfected with N-GLuc-YAP and C-GLuc-TEAD using TransIT-293 (Mirus Bio). The pCMV-Red Firefly Luc Vector (Life Technologies) was used as an internal control. Cells were treated with indicated concentrations of compounds or vehicle control (DMSO) in duplicates. Luciferase activity was measured by Dual-Luciferase Assay (Promega, Madison, WI) according to the manufacturer’s manual.

### Statistical Analyses

When comparing two groups, statistical significance was calculated by two-tailed unpaired t-test with Welch’s correction. One-way ANOVA with Dunnett’s multiple comparisons test was used when comparing three or more groups. Fisher’s exact test (two-sided) was used to analyze statistical significance for the odds ratio for YAP^{high} cells undergoing apoptosis in response to osimertinib/trametinib treatment. All statistical analyses and graphs were generated using GraphPad Prism 7.04 software.

### REFERENCES

1. Alexander, W.M., Ficarro, S.B., Adelmant, G., and Marto, J.A. (2017). multiplierz v2.0: A Python-based ecosystem for shared access and analysis of native mass spectrometry data. Proteomics 17, 15-16.

2. Bahcall, M., Sim, T., Paweletz, C.P., Patel, J.D., Alden, R.S., Kuang, Y., Sacher, A.G., Kim, N.D., Lydon, C.A., Awad, M.M., et al. (2016). Acquired METD1228Vmutation and resistance to MET inhibition in lung cancer. Cancer Discov. 6, 1334-1341.

3. Beyer, T.A., Weiss, A., Khomchuk, Y., Huang, K., Ogunjimi, A.A., Varelas, X., and Wrana, J.L. (2013). Switch enhancers interpret TGF-β and hippo signaling to control cell fate in human embryonic stem cells. Cell Rep. 5, 1611-1624.

4. Bhang, H.C., Ruddy, D.A., Krishnamurthy Radhakrishna, V., Caushi, J.X., Zhao, R., Hims, M.M., Singh, A.P., Kao, I., Rakiec, D., Shaw, P., et al. (2015). Studying clonal dynamics in response to cancer therapy using high-complexity barcoding. Nat. Med. 21, 440-448.

5. Bhola, P.D., and Letai, A. (2016). Mitochondria-Judges and Executioners of Cell Death Sentences. Mol. Cell 61, 695-704.

6. Blanke, C.D., Rankin, C., Demetri, G.D., Ryan, C.W., Von Mehren, M., Benjamin, R.S., Raymond, A.K., Bramwell, V.H.C., Baker, L.H., Maki, R.G., et al. (2008). Phase III randomized, intergroup trial assessing imatinib mesylate at two dose levels in patients with unresectable or metastatic gastrointestinal stromal tumors expressing the kit receptor tyrosine kinase: S0033. J. Clin. Oncol. 26, 626-632.

7. Buenrostro, J., Wu, B., Chang, H., and Greenleaf, W. (2015). ATAC-seq: A Method for Assaying Chromatin Accessibility Genome-Wide. Curr. Protoc. Mol. Biol. 109, 21.29.1-21.29.9.

8. Bum-Erdene, K., Zhou, D., Gonzalez-Gutierrez, G., Ghozayel, M.K., Si, Y., Xu, D., Shannon, H.E., Bailey, B.J., Corson, T.W., Pollok, K.E., et al. (2019). Small-Molecule Covalent Modification of Conserved Cysteine Leads to Allosteric Inhibition of the TEAD ▪ Yap Protein-Protein Interaction. Cell Chem. Biol. 26, 378-389.e13.

9. Byers, L.A., Diao, L., Wang, J., Saintigny, P., Girard, L., Peyton, M., Shen, L., Fan, Y., Giri, U., Tumula, P.K., et al. (2013). An epithelial-mesenchymal transition gene signature predicts resistance to EGFR and PI3K inhibitors and identifies Axl as a therapeutic target for overcoming EGFR inhibitor resistance. Clin. Cancer Res. 19, 279-290.

10. Campisi, J., and D’Adda Di Fagagna, F. (2007). Cellular senescence: When bad things happen to good cells. Nat. Rev. Mol. Cell Biol. 8, 729-740.

11. De Cesco, S., Kurian, J., Dufresne, C., Mittermaier, A.K., and Moitessier, N. (2017). Covalent inhibitors design and discovery. Eur. J. Med. Chem. 138, 96-114.

12. Chaib, I., Karachaliou, N., Pilotto, S., Codony Servat, J., Cai, X., Li, X., Drozdowskyj, A., Servat, C.C., Yang, J., Hu, C., et al. (2017). Co-activation of STAT3 and YES-Associated Protein 1 (YAP1) Pathway in EGFR-Mutant NSCLC. JNCI J. Natl. Cancer Inst. 109, 1-12.

13. Chan, P., Han, X., Zheng, B., Deran, M., Yu, J., Jarugumilli, G.K., Deng, H., Pan, D., Luo, X., and Wu, X. (2016). Autopalmitoylation of TEAD proteins regulates transcriptional output of the Hippo pathway. Nat. Chem. Biol. 12, 282-289.

14. Coppé, J.-P., Patil, C.K., Rodier, F., Sun, Y., Muñoz, D.P., Goldstein, J., Nelson, P.S., Desprez, P.-Y., and Campisi, J. (2008). Senescence-Associated Secretory Phenotypes Reveal Cell-Nonautonomous Functions of Oncogenic RAS and the p53 Tumor Suppressor. PLoS Biol. 6, e301.

15. Coppé, J.-P., Desprez, P.-Y., Krtolica, A., and Campisi, J. (2010). The Senescence-Associated Secretory Phenotype: The Dark Side of Tumor Suppression. Annu. Rev. Pathol. Mech. Dis. 5, 99-118.

16. Corces, M.R., Trevino, A.E., Hamilton, E.G., Greenside, P.G., Sinnott-Armstrong, N.A., Vesuna, S., Satpathy, A.T., Rubin, A.J., Montine, K.S., Wu, B., et al. (2017). An improved ATAC-seq protocol reduces background and enables interrogation of frozen tissues. Nat. Methods 14, 959-962.

17. Cornwell, M.I., Vangala, M., Taing, L., Herbert, Z., Köster, J., Li, B., Sun, H., Li, T., Zhang, J., Qiu, X., et al. (2018). VIPER: Visualization Pipeline for RNA-seq, a Snakemake workflow for efficient and complete RNA-seq analysis. BMC Bioinformatics 19, 1-14.

18. Cortot, A.B., and Jänne, P.A. (2014). Molecular mechanisms of resistance in epidermal growth factor receptor-mutant lung adenocarcinomas. Eur. Respir. Rev. 23, 356-366.

19. Costa, D.B., Halmos, B., Kumar, A., Schumer, S.T., Huberman, M.S., Boggon, T.J., Tenen, D.G., and Kobayashi, S. (2007). BIM mediates EGFR tyrosine kinase inhibitor-induced apoptosis in lung cancers with oncogenic EGFR mutations. PLoS Med. 4, 1669-1680.

20. Cragg, M.S., Kuroda, J., Puthalakath, H., Huang, D.C.S., and Strasser, A. (2007). Gefitinib-induced killing of NSCLC cell lines expressing mutant EGFR requires BIM and can be enhanced by BH3 mimetics. PLoS Med. 4, 1681-1690.

21. Debacq-Chainiaux, F., Erusalimsky, J.D., Campisi, J., and Toussaint, O. (2009). Protocols to detect senescence-associated beta-galactosidase (SA-βgal) activity, a biomarker of senescent cells in culture and in vivo. Nat. Protoc. 4, 1798-1806.

22. Drilon, A., Laetsch, T.W., Kummar, S., DuBois, S.G., Lassen, U.N., Demetri, G.D., Nathenson, M., Doebele, R.C., Farago, A.F., Pappo, A.S., et al. (2018). Efficacy of Larotrectinib in TRK Fusion-Positive Cancers in Adults and Children. N. Engl. J. Med. 378, 731-739.

23. Engelman, J. a, Zejnullahu, K., Mitsudomi, T., Song, Y., Hyland, C., Park, J.O., Lindeman, N., Gale, C.-M., Zhao, X., Christensen, J., et al. (2007). MET amplification leads to gefitinib resistance in lung cancer by activating ERBB3 signaling. Science 316, 1039-1043.

24. Ercan, D., Xu, C., Yanagita, M., Monast, C.S., Pratilas, C.A., Montero, J., Butaney, M., Shimamura, T., Sholl, L., Ivanova, E. V, et al. (2012). Reactivation of ERK signaling causes resistance to EGFR kinase inhibitors. Cancer Discov. 2, 934-947.

25. Ewald, J.A., Desotelle, J.A., Wilding, G., and Jarrard, D.F. (2010). Therapy-induced senescence in cancer. J. Natl. Cancer Inst. 102, 1536-1546.

26. Feldman, D., Tsai, F., Garrity, A.J., O’Rourke, R., Brenan, L., Ho, P., Gonzalez, E., Konermann, S., Johannessen, C.M., Beroukhim, R., et al. (2019). CloneRetriever: retrieval of rare clones from heterogeneous cell populations 2. BioRxiv https://doi.org/10.1101/762708.

27. Ficarro, S., Alexander, W., and Marto, J. (2017). mzStudio: A Dynamic Digital Canvas for User-Driven Interrogation of Mass Spectrometry Data. Proteomes 5, 20.

28. Ficarro, S.B., Browne, C.M., Card, J.D., Alexander, W.M., Zhang, T., Park, E., McNally, R., Dhe-Paganon, S., Seo, H.S., Lamberto, I., et al. (2016). Leveraging gas-phase fragmentation pathways for improved identification and selective detection of targets modified by covalent probes. Anal. Chem. 88, 12248-12254.

29. Fridman, A.L., and Tainsky, M.A. (2008). Critical pathways in cellular senescence and immortalization revealed by gene expression profiling. Oncogene 27, 5975-5987.

30. Gong, Y., Somwar, R., Politi, K., Balak, M., Chmielecki, J., Jiang, X., and Pao, W. (2007). Induction of BIM is essential for apoptosis triggered by EGFR kinase inhibitors in mutant EGFR-dependent lung adenocarcinomas. PLoS Med. 4, 1655-1668.

31. Guler, G.D., Tindell, C.A., Pitti, R., Wilson, C., Nichols, K., KaiWai Cheung, T., Kim, H.J., Wongchenko, M., Yan, Y., Haley, B., et al. (2017). Repression of Stress-Induced LINE-1 Expression Protects Cancer Cell Subpopulations from Lethal Drug Exposure. Cancer Cell 32, 221-237.e13.

32. Haferkamp, S., Borst, A., Adam, C., Becker, T.M., Motschenbacher, S., Windhövel, S., Hufnagel, A.L., Houben, R., and Meierjohann, S. (2013). Vemurafenib induces senescence features in melanoma cells. J. Invest. Dermatol. 133, 1601-1609.

33. Hata, A.N., Niederst, M.J., Archibald, H.L., Gomez-Caraballo, M., Siddiqui, F.M., Mulvey, H.E., Maruvka, Y.E., Ji, F., Bhang, H.C., Krishnamurthy Radhakrishna, V., et al. (2016). Tumor cells can follow distinct evolutionary paths to become resistant to epidermal growth factor receptor inhibition. Nat. Med. 22, 262-269.

34. Heinz, S., Benner, C., Spann, N., Bertolino, E., Lin, Y.C., Laslo, P., Cheng, J.X., Murre, C., Singh, H., and Glass, C.K. (2010). Simple combinations of lineage-determining transcription factors prime cis-regulatory elements required for macrophage and B cell identities. Mol. Cell 38, 576-589.

35. Hsu, P.-C., You, B., Yang, Y.-L., Zhang, W.-Q., Wang, Y.-C., Xu, Z., Dai, Y., Liu, S., Yang, C.-T., Li, H., et al. (2016). YAP promotes erlotinib resistance in human non-small cell lung cancer cells. Oncotarget 5.

36. Jamal-Hanjani, M., Wilson, G.A., McGranahan, N., Birkbak, N.J., Watkins, T.B.K., Veeriah, S., Shafi, S., Johnson, D.H., Mitter, R., Rosenthal, R., et al. (2017). Tracking the Evolution of Non-Small-Cell Lung Cancer. N. Engl. J. Med. 376, 2109-2121.

37. Kapoor, A., Yao, W., Ying, H., Hua, S., Liewen, A., Wang, Q., Zhong, Y., Wu, C.J., Sadanandam, A., Hu, B., et al. (2014). Yap1 activation enables bypass of oncogenic KRAS addiction in pancreatic cancer. Cell 158, 185-197.

38. Kim, M., Kim, T., Johnson, R.L., and Lim, D.S. (2015). Transcriptional co-repressor function of the hippo pathway transducers YAP and TAZ. Cell Rep. 11, 270-282.

39. Ku, G., Tan, I.B., Yau, T., Boku, N., Laohavinij, S., Cheng, A.-L., Kang, Y.-K., and de Lima Lopes, G. (2012). Management of colon cancer: resource-stratified guidelines from the Asian Oncology Summit 2012. Lancet. Oncol. 13, e470-81.

40. Kuwana, T., Bouchier-Hayes, L., Chipuk, J.E., Bonzon, C., Sullivan, B.A., Green, D.R., and Newmeyer, D.D. (2005). BH3 domains of BH3-only proteins differentially regulate Bax-mediated mitochondrial membrane permeabilization both directly and indirectly. Mol. Cell 17, 525-535.

41. Lehmann, W., Mossmann, D., Kleemann, J., Mock, K., Meisinger, C., Brummer, T., Herr, R., Brabletz, S., Stemmler, M.P., and Brabletz, T. (2016). ZEB1 turns into a transcriptional activator by interacting with YAP1 in aggressive cancer types. Nat. Commun. 7, 1-15.

42. Lei, Q.-Y., Zhang, H., Zhao, B., Zha, Z.-Y., Bai, F., Pei, X.-H., Zhao, S., Xiong, Y., and Guan, K.-L. (2008). TAZ Promotes Cell Proliferation and Epithelial-Mesenchymal Transition and Is Inhibited by the Hippo Pathway. Mol. Cell. Biol. 28, 2426-2436.

43. Ley, R., Ewings, K.E., Hadfield, K., and Cook, S.J. (2005). Regulatory phosphorylation of Bim: Sorting out the ERK from the JNK. Cell Death Differ. 12, 1008-1014.

44. Li, H., and Durbin, R. (2010). Fast and accurate long-read alignment with Burrows-Wheeler transform. Bioinformatics 26, 589-595.

45. Liberzon, A., Birger, C., Thorvaldsdottir, H., Ghandi, M., Mesirov, J.P., and Tamayo, P. (2015). The Molecular Signatures Database Hallmark Gene Set Collection. Cell Syst. 1, 417-425.

46. Lin, L., Sabnis, A.J., Chan, E., Olivas, V., Cade, L., Pazarentzos, E., Asthana, S., Neel, D., Yan, J.J., Lu, X., et al. (2015). The Hippo effector YAP promotes resistance to RAF- and MEK-targeted cancer therapies. Nat. Genet. 47, 250-256.

47. Liu, Q., Sabnis, Y., Zhao, Z., Zhang, T., Buhrlage, S.J., Jones, L.H., and Gray, N.S. (2013). Developing irreversible inhibitors of the protein kinase cysteinome. Chem. Biol. 20, 146-159.

48. Mohseni, M., Sun, J., Lau, A., Curtis, S., Goldsmith, J., Fox, V.L., Wei, C., Frazier, M., Samson, O., Wong, K.K., et al. (2014). A genetic screen identifies an LKB1-MARK signalling axis controlling the Hippo-YAP pathway. Nat. Cell Biol. 16, 108-117.

49. Mok, T.S., Wu, Y., Thongprasert, S., Yang, C., Saijo, N., Sunpaweravong, P., Han, B., Margono, B., Ichinose, Y., Nishiwaki, Y., et al. (2009). Gefitinib or Carboplatin-Paclitaxel in Pulmonary Adenocarcinoma. N. Engl. J. Med. 361, 947-957.

50. Mok, T.S., Wu, Y.-L., Ahn, M.-J., Garassino, M.C., Kim, H.R., Ramalingam, S.S., Shepherd, F.A., He, Y., Akamatsu, H., Theelen, W.S.M.E., et al. (2017). Osimertinib or Platinum-Pemetrexed in EGFR T790M-Positive Lung Cancer. N. Engl. J. Med. 376, 629-640.

51. Nardella, C., Clohessy, J.G., Alimonti, A., and Pandolfi, P.P. (2011). Pro-senescence therapy for cancer treatment. Nat. Rev. Cancer 11, 503-511.

52. Narita, M., Nun, S., Heard, E., Narita, M., Lin, A.W., Hearn, S.A., Spector, D.L., Hannon, G.J., Lowe, S.W., Brook, S., et al. (2003). Rb-Mediated Heterochromatin Formation and Silencing of E2F Target Genes during Cellular Senescence State University of New York at Stony Brook. Cell 113, 703-716.

53. Noland, C.L., Gierke, S., Schnier, P.D., Murray, J., Sandoval, W.N., Sagolla, M., Dey, A., Hannoush, R.N., Fairbrother, W.J., and Cunningham, C.N. (2016). Palmitoylation of TEAD Transcription Factors Is Required for Their Stability and Function in Hippo Pathway Signaling. Structure 24, 179-186.

54. Peters, S., Camidge, D.R., Shaw, A.T., Gadgeel, S., Ahn, J.S., Kim, D.-W., Ou, S.-H.I., Pérol, M., Dziadziuszko, R., Rosell, R., et al. (2017). Alectinib versus Crizotinib in Untreated ALK -Positive Non-Small-Cell Lung Cancer. N. Engl. J. Med. 377, 829-838.

55. Piccolo, S., Dupont, S., and Cordenonsi, M. (2014). The biology of YAP/TAZ: hippo signaling and beyond. Physiol. Rev. 94, 1287-1312.

56. Pobbati, A. V., Han, X., Hung, A.W., Weiguang, S., Huda, N., Chen, G.Y., Kang, C.B., Chia, C.S.B., Luo, X., Hong, W., et al. (2015). Targeting the Central Pocket in Human Transcription Factor TEAD as a Potential Cancer Therapeutic Strategy. Structure 23, 2076-2086.

57. Qin, Q., Mei, S., Wu, Q., Sun, H., Li, L., Taing, L., Chen, S., Li, F., Liu, T., Zang, C., et al. (2016). ChiLin: A comprehensive ChIP-seq and DNase-seq quality control and analysis pipeline. BMC Bioinformatics 17, 1-13.

58. Ramalingam, S., Saka, H., Ahn, M.-J., Yu, H., Horn, L., Hida, T., Cantarini, M., Verheijen, R., Wessen, J., Oxnard, G., et al. (2019). Osimertinib plus selumetinib for patients with EGFR-mutant (EGFRm) NSCLC following disease progression on an EGFR-TKI: results from the Phase Ib TATTON study. In AACR Annual Meeting 2019, (Atlanta (GA): AACR), p.

59. Ramirez, F., Ryan, D.P., Grüning, B., Bhardwaj, V., Kilpert, F., Richter, A.S., Heyne, S., Dündar, F., and Manke, T. (2016). deepTools2: a next generation web server for deep-sequencing data analysis. Nucleic Acids Res. 44, W160-W165.

60. Richardson, C.D., Ray, G.J., DeWitt, M.A., Curie, G.L., and Corn, J.E. (2016). Enhancing homology-directed genome editing by catalytically active and inactive CRISPR-Cas9 using asymmetric donor DNA. Nat. Biotechnol. 34, 339-344.

61. Rosell, R., Carcereny, E., Gervais, R., Vergnenegre, A., Massuti, B., Felip, E., Palmero, R., Garcia-Gomez, R., Pallares, C., Sanchez, J.M., et al. (2012). Erlotinib versus standard chemotherapy as first-line treatment for European patients with advanced EGFR mutation-positive non-small-cell lung cancer (EURTAC): a multicentre, open-label, randomised phase 3 trial. Lancet Oncol. 13, 239-246.

62. Rosenbluh, J., Nijhawan, D., Cox, A.G., Li, X., Neal, J.T., Schafer, E.J., Zack, T.I., Wang, X., Tsherniak, A., Schinzel, A.C., et al. (2012). β-Catenin-driven cancers require a YAP1 transcriptional complex for survival and tumorigenesis. Cell 151, 1457-1473.

63. Sequist, L. V, Waltman, B.A., Dias-Santagata, D., Digumarthy, S., Turke, A.B., Fidias, P., Bergethon, K., Shaw, A.T., Gettinger, S., Cosper, A.K., et al. (2011). Genotypic and histological evolution of lung cancers acquiring resistance to EGFR inhibitors. Sci. Transl. Med. 3, 75ra26.

64. Shao, Y., and Aplin, A.E. (2010). Akt3-mediated resistance to apoptosis in B-RAF-targeted melanoma cells. Cancer Res. 70, 6670-6681.

65. Shao, Y., and Aplin, A.E. (2012). ERK2 phosphorylation of serine 77 regulates Bmf pro-apoptotic activity. Cell Death Dis. 3, e253-10.

66. Shao, D.D., Xue, W., Krall, E.B., Bhutkar, A., Piccioni, F., Wang, X., Schinzel, A.C., Sood, S., Rosenbluh, J., Kim, J.W., et al. (2014). KRAS and YAP1 converge to regulate EMT and tumor survival. Cell 158, 171-184.

67. Sharma, S. V., Lee, D.Y., Li, B., Quinlan, M.P., Takahashi, F., Maheswaran, S., McDermott, U., Azizian, N., Zou, L., Fischbach, M.A., et al. (2010). A Chromatin-Mediated Reversible Drug-Tolerant State in Cancer Cell Subpopulations. Cell 141, 69-80.

68. Shibue, T., and Weinberg, R.A. (2017). EMT, CSCs, and drug resistance: The mechanistic link and clinical implications. Nat. Rev. Clin. Oncol. 14, 611-629.

69. Soria, J.-C., Ohe, Y., Vansteenkiste, J., Reungwetwattana, T., Chewaskulyong, B., Lee, K.H., Dechaphunkul, A., Imamura, F., Nogami, N., Kurata, T., et al. (2018). Osimertinib in Untreated EGFR -Mutated Advanced Non-Small-Cell Lung Cancer. N. Engl. J. Med. 378, 113-125.

70. Sudol, M. (2012). YAP1 oncogene and its eight isoforms. Oncogene 32, 3922.

71. Tang, Y., Feinberg, T., Keller, E.T., Li, X.Y., and Weiss, S.J. (2016). Snail/Slug binding interactions with YAP/TAZ control skeletal stem cell self-renewal and differentiation. Nat. Cell Biol. 18, 917-929.

72. Thress, K.S., Jacobs, V., Angell, H.K., Yang, J.C.H., Sequist, L. V., Blackhall, F., Su, W.C., Schuler, M., Wolf, J., Gold, K.A., et al. (2017). Modulation of Biomarker Expression by Osimertinib: Results of the Paired Tumor Biopsy Cohorts of the AURA Phase I Trial. J. Thorac. Oncol. 12, 1588-1594.

73. Tricker, E.M., Xu, C., Uddin, S., Capelletti, M., Ercan, D., Ogino, A., Pratilas, C.A., Rosen, N., Gray, N.S., Wong, K., et al. (2015). Combined EGFR/MEK Inhibition Prevents the Emergence of Resistance in EGFR-Mutant Lung Cancer. Cancer Discov. 5, 960-971.

74. Wang, W., Li, N., Li, X., Tran, M.K., Han, X., and Chen, J. (2015). Tankyrase Inhibitors Target YAP by Stabilizing Angiomotin Family Proteins. Cell Rep. 13, 524-532.

75. Wu, C.-H., van Riggelen, J., Yetil, A., Fan, A.C., Bachireddy, P., and Felsher, D.W. (2007). Cellular senescence is an important mechanism of tumor regression upon c-Myc inactivation. Proc. Natl. Acad. Sci. 104, 13028-13033.

76. Zaidi, S.K., Sullivan, A.J., Medina, R., Ito, Y., van Wijnen, A.J., Stein, J.L., Lian, J.B., and Stein, G.S. (2004). Tyrosine phosphorylation controls Runx2-mediated subnuclear targeting of YAP to repress transcription. EMBO J. 23, 790-799.

77. Zanconato, F., Cordenonsi, M., and Piccolo, S. (2016). YAP/TAZ at the Roots of Cancer. Cancer Cell 29, 783-803.

78. Zhang, Z., and Marshall, A.G. (1998). A universal algorithm for fast and automated charge state deconvolution of electrospray mass-to-charge ratio spectra. J. Am. Soc. Mass Spectrom. 9, 225-233.

79. Zhang, H., Liu, C.Y., Zha, Z.Y., Zhao, B., Yao, J., Zhao, S., Xiong, Y., Lei, Q.Y., and Guan, K.L. (2009). TEAD transcription factors mediate the function of TAZ in cell growth and epithelial-mesenchymal transition. J. Biol. Chem. 284, 13355-13362.

80. Zhang, Y., Liu, T., Meyer, C.A., Eeckhoute, J., Johnson, D.S., Bernstein, B.E., Nussbaum, C., Myers, R.M., Brown, M., Li, W., et al. (2008). Model-based analysis of ChIP-Seq (MACS). Genome Biol. 9.

81. Zhang, Z., Lee, J.C., Lin, L., Olivas, V., Au, V., Laframboise, T., Abdel-Rahman, M., Wang, X., Levine, A.D., Rho, J.K., et al. (2012). Activation of the AXL kinase causes resistance to EGFR-targeted therapy in lung cancer. Nat. Genet. 44, 852-860.

82. Zhao, B., Ye, X., Yu, J., Li, L., Li, W., Li, S., Yu, J., Lin, J.D., Wang, C.-Y., Chinnaiyan, A.M., et al. (2008). TEAD mediates YAP-dependent gene induction and growth control. Genes Dev. 22, 1962-1971.

83. Zhou, W., Ercan, D., Chen, L., Yun, C.-H., Li, D., Capelletti, M., Cortot, A.B., Chirieac, L., Iacob, R.E., Padera, R., et al. (2009). Novel mutant-selective EGFR kinase inhibitors against EGFR T790M. Nature 462, 1070-1074.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I-A): 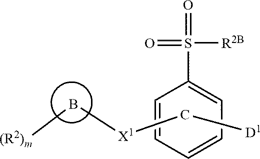
or a pharmaceutically acceptable salt, co-crystal, tautomer, stereoisomer, solvate, hydrate, polymorph, isotopically enriched derivative, or prodrug thereof, wherein:
Ring B is cyclohexyl or phenyl;
R² is halogen, optionally substituted acyl, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, —OR^{c1}, —NO₂, —N(R^{c2})₂, —SR^{c1}, —CN, or —SCN,
wherein R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{c2} are taken together with their intervening atoms to form a substituted or unsubstituted heterocyclic or substituted or unsubstituted heteroaryl ring;
R^{2B} is -N(R^{c2})₂, -OR^{c1}, optionally substituted alkyl, optionally substituted alkenyl, optionally substituted alkynyl, optionally substituted carbocyclyl, optionally substituted heterocyclyl, optionally substituted aryl, or optionally substituted heteroaryl;
X¹ is —O—, -O(alkylene)-, alkylene, —S—, —SCH₂—, —N(R^{da})—, or —N(R^{da})CH₂—;
R^{da} is hydrogen, optionally substituted C₁₋₆ alkyl, optionally substituted acyl, or a nitrogen protecting group;
m is 0, 1, 2, 3, 4, 5, 6, 7, 8, 9, or 10; and
D¹ is a warhead of any one of Formulae (i-1) to (i-23), (i-26) to (i-31), (i-34) to (i-40), (i-42), or (i-43): 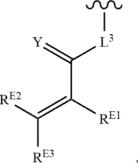 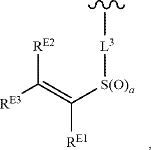 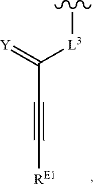 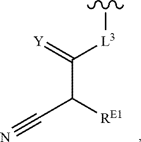 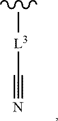 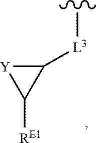 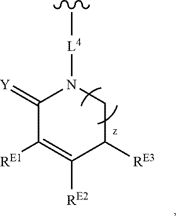 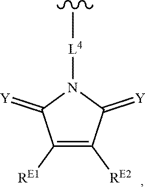 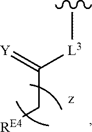 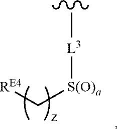 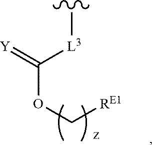 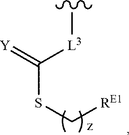 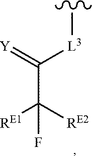 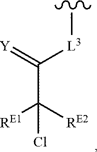 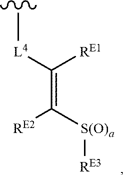 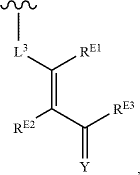 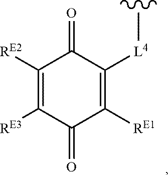 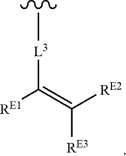 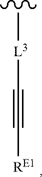 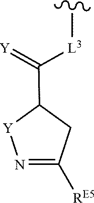 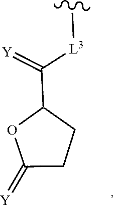 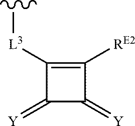 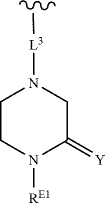 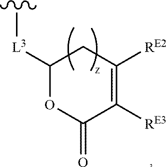 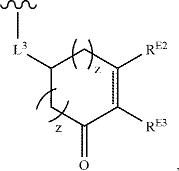 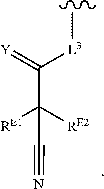 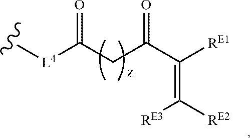 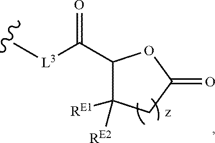 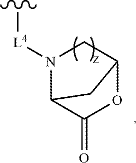 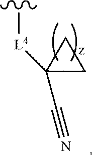 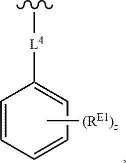 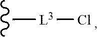 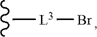 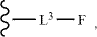 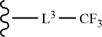 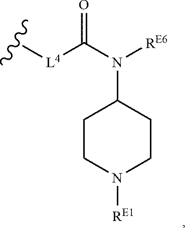 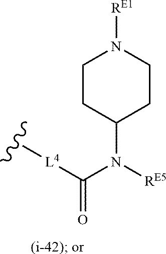 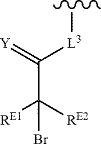
wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}——SC(═O)—, —C(═O)S—, —OC(═O)—, —C(═O)O—, —NR^{L3a}C(═S)—, —C(═S)NR^{L3a}—, trans-CR^{L3b}=CR^{L3b}-, cis-CR^{L3b}=CR^{L3b}-, —C═C—, —S(═O)—, —S(═O)O—, —OS(═O)—, —S(═O)NRL3a—, —NR^{L3a}S(═O)_{—}, —S(═O)₂—, —S(═O)₂0—, —OS(═O)₂—, —S(═O)₂NR^{L3a}—, or —NR^{L3a}S(—O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits.

2. A compound of Formula (I-B): 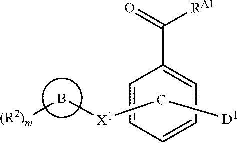
or a pharmaceutically acceptable salt, co-crystal, tautomer, stereoisomer, solvate, hydrate, polymorph, isotopically enriched derivative, or prodrug thereof, wherein:
R^{A1} is —O(R^{a2}) or —N(R^{a3})₂;
R^{a2} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group; and
each instance of R^{a3} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —SO₂(R^{a4}), or a nitrogen protecting group, or optionally two instances of R^{a3} are taken together with their intervening atoms to form a substituted or unsubstituted heterocyclic or substituted or unsubstituted heteroaryl ring; and
R^{a4} is optionally substituted alkyl, optionally substituted alkenyl, optionally substituted alkynyl, optionally substituted carbocyclyl, optionally substituted heterocyclyl, optionally substituted aryl, or optionally substituted heteroaryl;
Ring B is cyclohexyl or phenyl;
R² is halogen, optionally substituted acyl, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, —OR^{c1}, —NO₂, —N(R^{c2})₂, —SR^{c1}, —CN, or —SCN,
wherein R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{c2} are taken together with their intervening atoms to form a substituted or unsubstituted heterocyclic or substituted or unsubstituted heteroaryl ring;
X¹ is —O—, -O(alkylene)-, alkylene, —S—, —SCH₂—, -N(R^{da})-, or -N(R^{da})CH₂-;
R^{da} is hydrogen, optionally substituted C₁₋₆ alkyl, or a nitrogen protecting group;
m is 0, 1, 2, 3, 4, 5, 6, 7, 8, 9, or 10; and
D¹ is a warhead of any one of Formulae (i-1) to (i-23), (i-26) to (i-31), (i-34) to (i-40), (i-42), or (i-43): 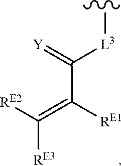 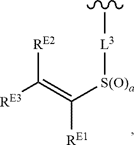 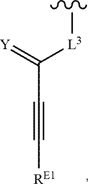 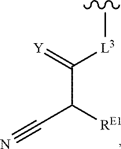 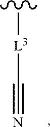 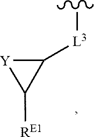 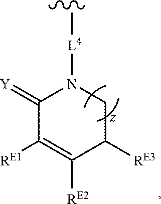 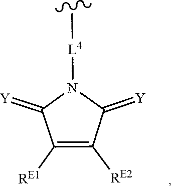 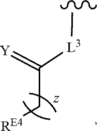 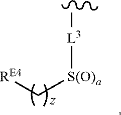 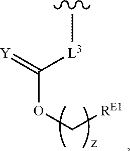 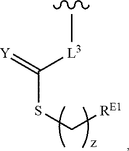 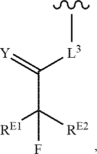 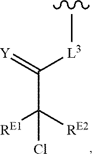 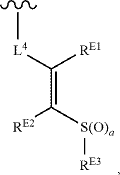 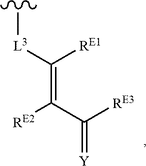 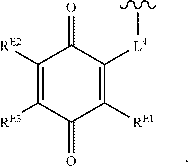 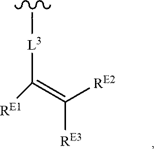 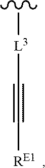 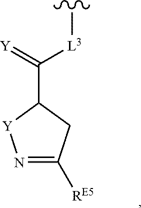 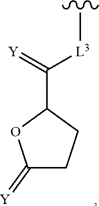 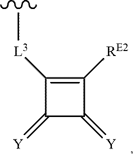 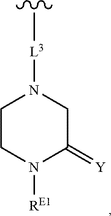 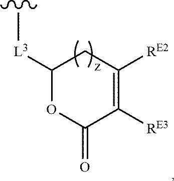 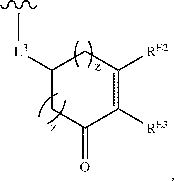 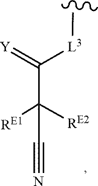 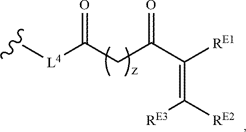 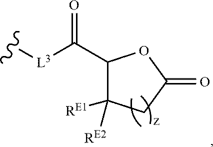 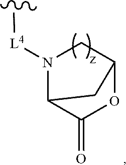 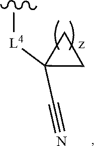 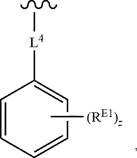 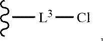 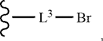 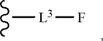 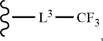 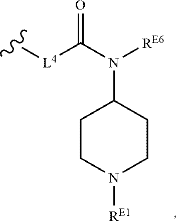 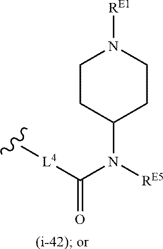 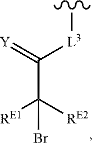
wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, -NR^{L3a}C(═O)—, -C(═O)NR^{L3a}——SC(═O)—, —C(═O)S—, —OC(═O)—, —C(═O)O—, —NR^{L3a}C(═S)—, —C(═S)NR^{L3a}—, trans-CR^{L3b}=CR^{L3b}-, cis-CR^{L3b}=CR^{L3b}-, —C═C—, —S(═O)—, —S(═O)O—, —OS(═O)—, —S(═O)NR^{L3a}—, —NR^{L3a}S(═O)—, —S(═O)₂—, —S(═O)₂0—, —OS(═O)₂—, —S(═O)₂NR^{L3a}—, or —NR^{L3a}S(—O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or -SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits;
provided that the compound is not of formula: 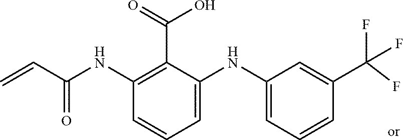 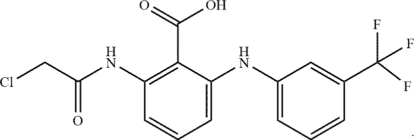
.

3. The compound of claim 1, wherein the compound is of formula: 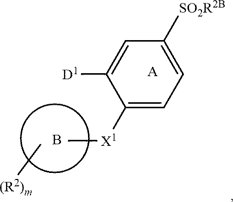
or a pharmaceutically acceptable salt, solvate, hydrate, polymorph, co-crystal, tautomer, stereoisomer, isotopically labeled derivative, or prodrug thereof.

4. The compound of any one of claims 1-3, wherein Ring B is cyclohexyl.

5. The compound of any one of claims 1-3, wherein Ring B is phenyl.

6. The compound of any one of claims 1-5, wherein m is 0.

7. The compound of any one of claims 1-5, wherein m is 1.

8. The compound of any one of claims 1-7, wherein Ring B is of formula: 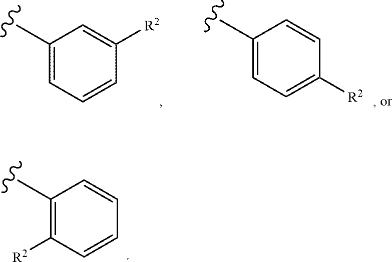
.

9. The compound of any one of claims 1-8, wherein Ring B is of formula: 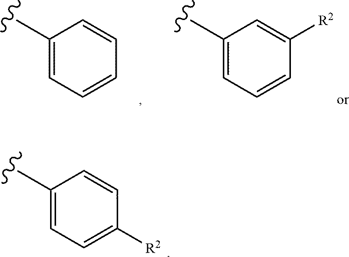
.

10. The compound of any one of claims 1-9, wherein at least one instance of R² is optionally substituted alkyl.

11. The compound of claim 10, wherein at least one instance of R² is optionally substituted C₁₋₆ alkyl.

12. The compound of claim 10, wherein at least one instance of R² is —CF₃.

13. The compound of any one of claims 1-4, 6, 7, or 10-12, wherein the moiety 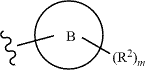
is of formula: 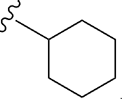
.

14. The compound of any one of claims 1-3, 5, or 7-12, wherein the moiety 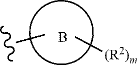
is of formula: 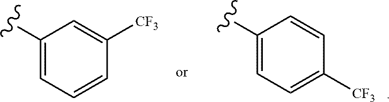
.

15. The compound of any one of claims 1 or 3-14, wherein R^{2B} is —N(R^{c2})₂, and each instance of R^{c2} is independently hydrogen, optionally substituted alkyl, or optionally substituted carbocyclyl.

16. The compound of claim 15, wherein R^{2B} is —N(R^{c2})₂, and at least one instance of R^{c2} is hydrogen.

17. The compound of claim 15 or 16, wherein R^{2B} is -NH(R^{c2}), wherein R^{c2} is optionally substituted C₁₋₆ alkyl or optionally substituted C₃₋₁₀ carbocyclyl.

18. The compound of any one of claims 1 or 3-16, wherein R^{2B} is —NHMe or 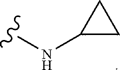
.

19. The compound of compound of any one of claims 1 or 3-16, wherein R^{2B} is optionally substituted alkyl.

20. The compound of compound of claim 19, wherein R^{2B} is optionally substituted methyl.

21. The compound of claim 2, wherein R^{A1} is —OR^{a2}, and R^{a2} is hydrogen or optionally substituted alkyl.

22. The compound of claim 2 or 21, wherein R^{A1} is —OH.

23. The compound of claim 2, wherein R^{A1} is —N(R^{a3})₂, wherein at least one instance of R^{a3} is hydrogen, optionally substituted C₁₋₆ alkyl, optionally substituted C₃₋₁₀ carbocyclyl, or —SO₂(R^{a4}), and R^{a4} is optionally substituted alkyl, optionally substituted alkenyl, optionally substituted alkynyl, or optionally substituted carbocyclyl.

24. The compound of claim 2 or 23, wherein R^{A1} is —NH(R^{a3}).

25. The compound of any one of claims 2, 23, or 24, wherein R^{A1} is 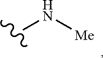 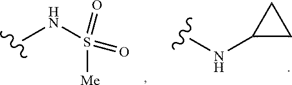
, or —NMe₂.

26. The compound of any one of claims 1-25, wherein X¹ is —O—.

27. The compound of any one of claims 1-25, wherein X¹ is —O(CR^{d})_{1—6}—, and R^{d} is hydrogen, halogen, optionally substituted acyl, optionally substituted alkyl, optionally substituted alkenyl, optionally substituted alkynyl, optionally substituted carbocyclyl, —OR^{c1}, —NO₂, -N(R^{c2})₂, -SR^{c1}, —CN, or —SCN.

28. The compound of claim 27, wherein X¹ is of formula: 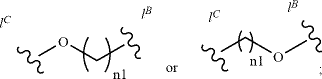
l^{c} indicates the point of attachment to the moiety of formula: 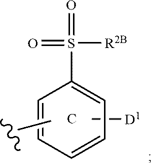
and l^{B} indicates the point of attachment to Ring B; and
n1 is 1, 2, 3, 4, 5, or 6.

29. The compound of claim 27, wherein X¹ is of formula: 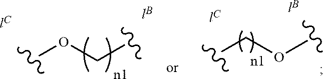
l^{C} indicates the point of attachment to the moiety of formula: 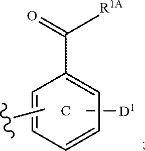
and l^{B} indicates the point of attachment to Ring B; and
n1 is 1, 2, 3, 4, 5, or 6.

30. The compound of any one of claims 27-29, wherein X¹ is of formula: 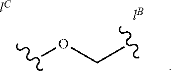
.

31. The compound of any one of claims 1-25, wherein X¹ is —N(R^{da})—, and R^{da} is hydrogen or optionally substituted C₁₋₆ alkyl.

32. The compound of claim 30, wherein X¹ is —NH—.

33. The compound of any one of claims 1-32, wherein D¹ is of formula: 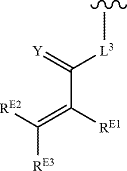
.

34. The compound of any one of claims 1-32, wherein D¹ is of formula 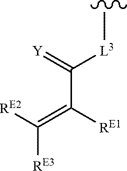
wherein:
L³ is a bond or optionally substituted C₁₋₄ alkyl, and optionally wherein 1 to 2 carbon units of the C₁₋₄ alkyl are replaced with —C═O—, -NR^{L3a}-, or —NR^{L3a}C(—O)—;
R^{L3a} is hydrogen, substituted or unsubstituted C₁₋₆ alkyl, or a nitrogen protecting group; Y is O; and
each of R^{E1}, R^{E2}, and R^{E3} is independently hydrogen, halogen, optionally substituted alkyl, optionally substituted alkenyl, or optionally substituted alkynyl.

35. The compound of any one of claims 1-34, wherein D¹ is of formula: 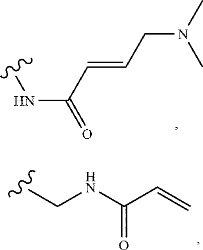 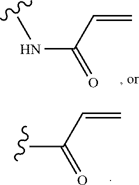
.

36. The compound of any one of claims 1-35, wherein D¹ is of formula: 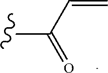
.

37. The compound of any one of claims 1-35, wherein D¹ is of formula: 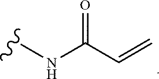
.

38. The compound of any one of claims 1, 3-20, 26-28, or 30-37, wherein the moiety of formula: 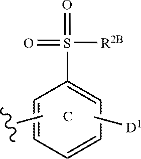
is of formula: 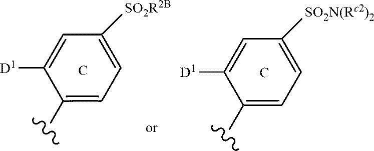
.

39. The compound of any one of claims 1, 3-20, 26-28, or 30-38, wherein the compound is of formula: 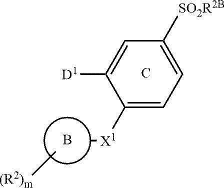
or a pharmaceutically acceptable salt, solvate, hydrate, polymorph, co-crystal, tautomer, stereoisomer, isotopically labeled derivative, or prodrug thereof.

40. The compound of any one of claims 1, 3-20, 26-28, or 30-39, wherein the compound is of formula: 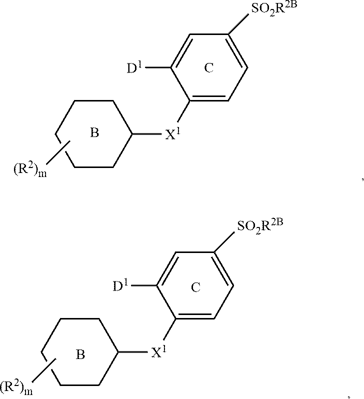
or a pharmaceutically acceptable salt, solvate, hydrate, polymorph, co-crystal, tautomer, stereoisomer, isotopically labeled derivative, or prodrug thereof.

41. The compound of any one of claims 1, 3-20, 26-28, or 30-40, wherein the compound is of formula: 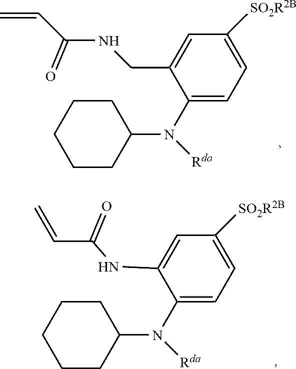 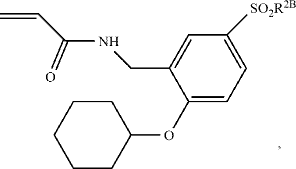 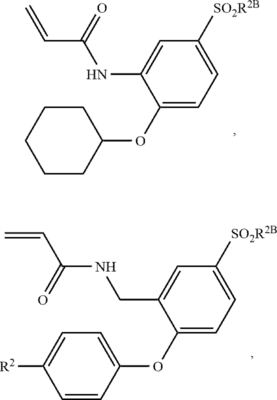 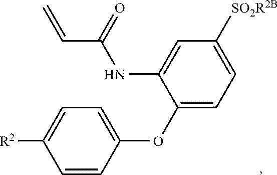 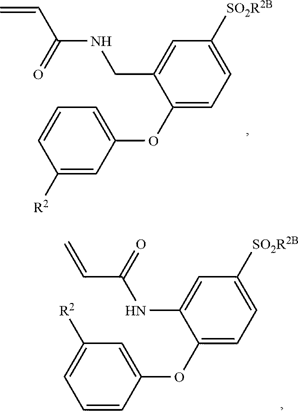 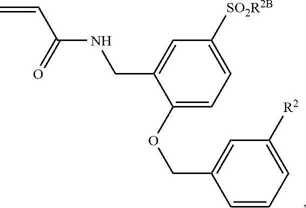 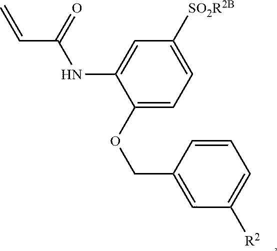
or a pharmaceutically acceptable salt, solvate, hydrate, polymorph, co-crystal, tautomer, stereoisomer, isotopically labeled derivative, or prodrug thereof.

42. The compound of claim 1, wherein the compound is of formula: 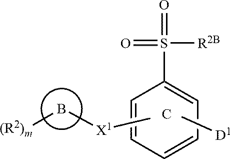
or a pharmaceutically acceptable salt thereof.

43. The compound of any one of claims 1, 3-20, 28, or 30-42, wherein the compound is of formula: 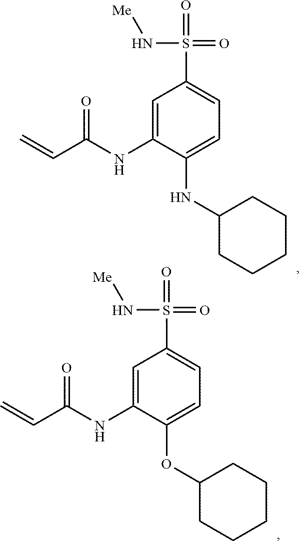 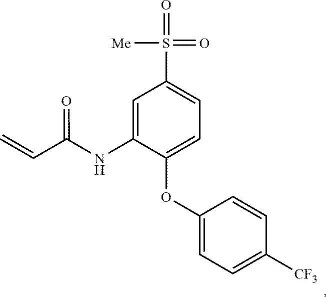 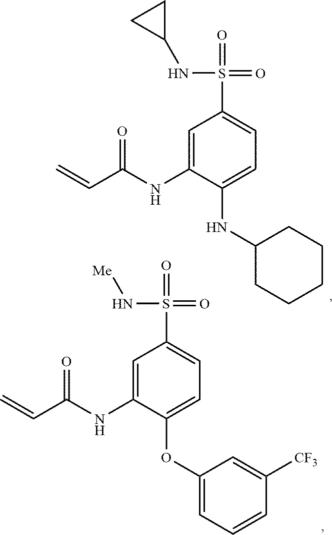 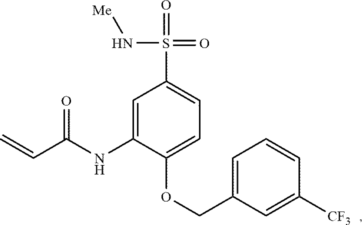
or a pharmaceutically acceptable salt, co-crystal, tautomer, stereoisomer, solvate, hydrate, polymorph, isotopically enriched derivative, or prodrug thereof.

44. The compound of any one of claims 2-14, 20-27, or 29-37, wherein the moiety of formula: 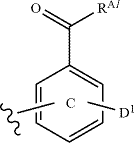
is of formula: 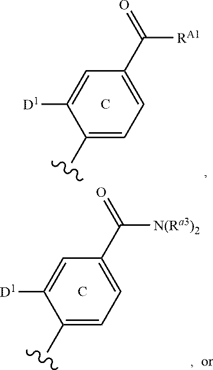 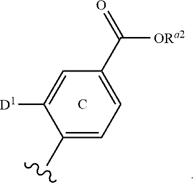
.

45. The compound of any one of claims 2-14, 20-27, 29-37, or 44, wherein the compound is of formula: 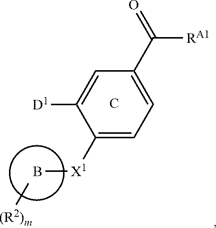
or a pharmaceutically acceptable salt, solvate, hydrate, polymorph, co-crystal, tautomer, stereoisomer, isotopically labeled derivative, or prodrug thereof.

46. The compound of any one of claims 2-14, 20-27, 29-37, or 42-45, wherein the compound is of formula: 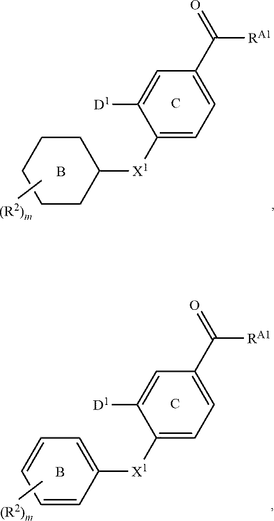
or a pharmaceutically acceptable salt, solvate, hydrate, polymorph, co-crystal, tautomer, stereoisomer, isotopically labeled derivative, or prodrug thereof.

47. The compound of any one of claims 2-14, 20-27, 29-37, or 42-46, wherein the compound is of formula: 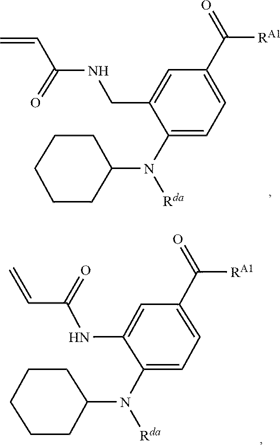 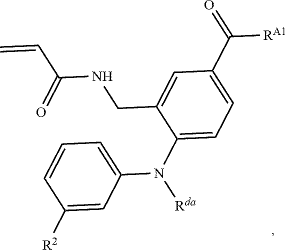 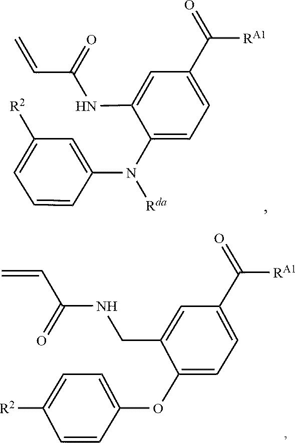 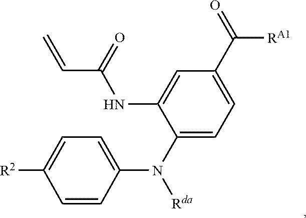 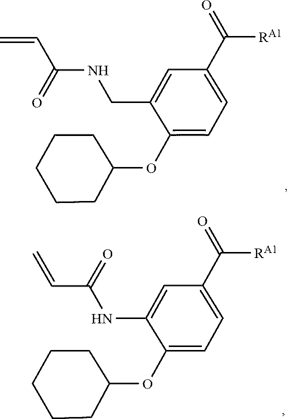 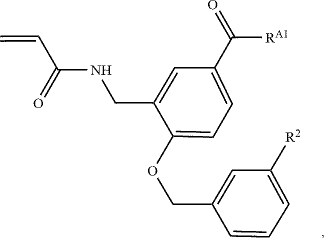 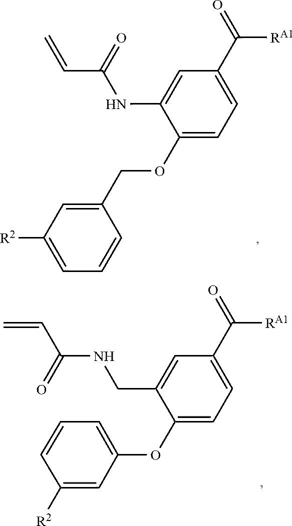 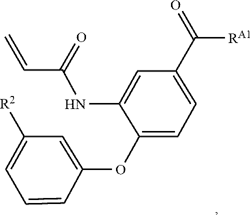
or a pharmaceutically acceptable salt, solvate, hydrate, polymorph, co-crystal, tautomer, stereoisomer, isotopically labeled derivative, or prodrug thereof.

48. The compound of claim 2, wherein the compound is of formula: 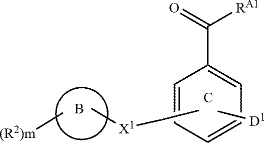
or a pharmaceutically acceptable salt thereof.

49. The compound of any one of claims 2-14, 20-27, 29-37, or 42-48, wherein the compound is of formula: 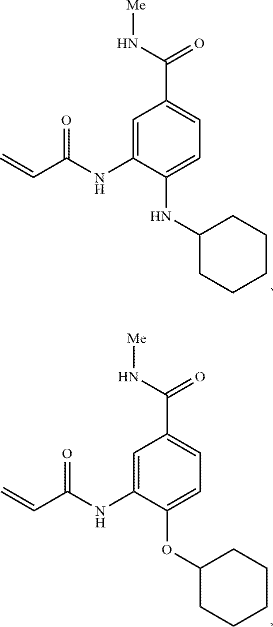 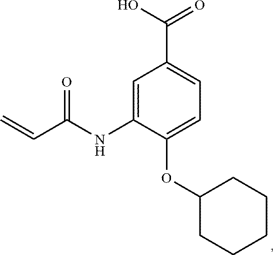 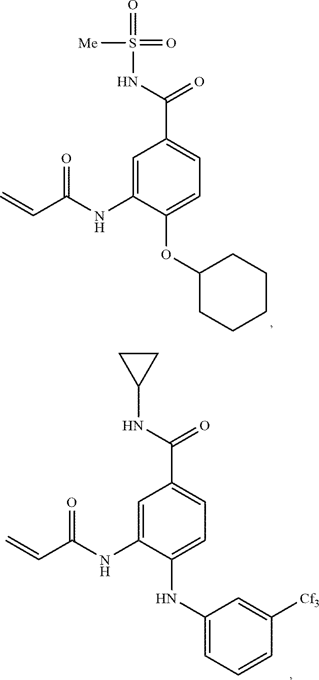 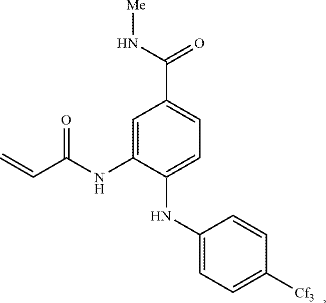 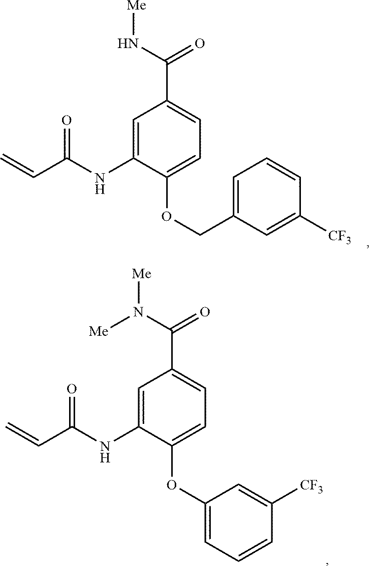
or a pharmaceutically acceptable salt, co-crystal, tautomer, stereoisomer, solvate, hydrate, polymorph, isotopically enriched derivative, or prodrug thereof.

50. The compound of any one of claims 1-49, wherein the compound is of formula: 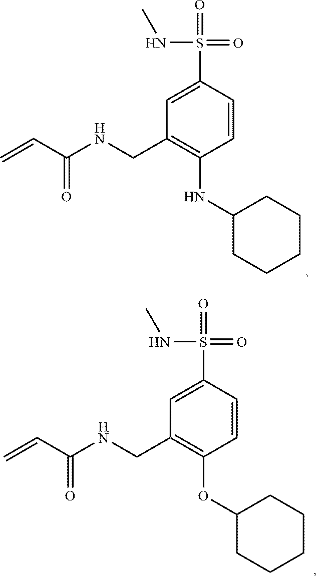 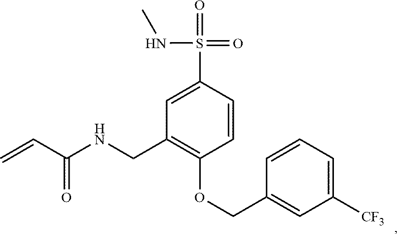 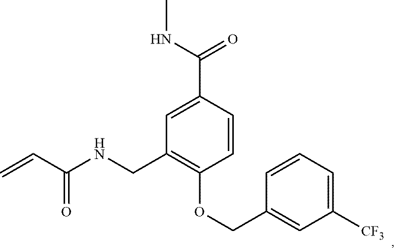
or a pharmaceutically acceptable salt, co-crystal, tautomer, stereoisomer, solvate, hydrate, polymorph, isotopically enriched derivative, or prodrug thereof.

51. A compound of Formula (II): 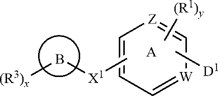
or a pharmaceutically acceptable salt, co-crystal, tautomer, stereoisomer, solvate, hydrate, polymorph, isotopically enriched derivative, or prodrug thereof, wherein:
Ring B is cyclohexyl or phenyl;
W is —C(R^{a})═ or —N═, as valency permits; and R^{a} is hydrogen, halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN;
Z is —C(R^{b})═ or —N═, as valency permits; and R^{b} is hydrogen, halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN;
provided at least one instance of W and Z is —C(R^{a})═ or —C(R^{b})═;
each instance of R¹ is independently halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN;
each instance of R³ is independently halogen, optionally substituted acyl, optionally substituted alkyl, optionally substituted alkenyl, optionally substituted alkynyl, optionally substituted carbocyclyl, —OR^{c1}, —NO₂, —N(R^{c2})₂, —SR^{c1}, —CN, or —SCN;
wherein R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{c2} are taken together with their intervening atoms to form a substituted or unsubstituted heterocyclic or substituted or unsubstituted heteroaryl ring;
X¹ is —O—, -O(alkylene)-, alkylene, —S—, —SCH₂—, -N(R^{da})—, or —N(R^{da})CH₂—;
R^{da} is hydrogen, optionally substituted C₁₋₆ alkyl, optionally substituted acyl, or a nitrogen protecting group;
x is 0, 1, 2, 3, 4, 5, 6, 7, 8, 9, or 10;
y is 0, 1, 2, 3, or 4;
D¹ is a warhead of any one of Formulae (i-1) to (i-23), (i-26) to (i-31), (i-34) to (i-40), (i-42), or (i-43): 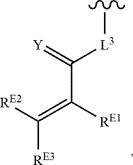 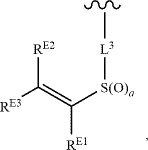 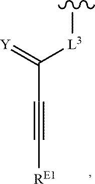 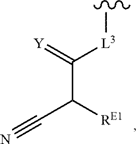 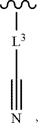 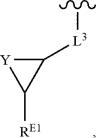 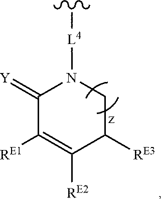 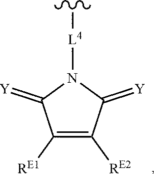 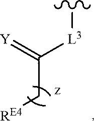 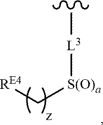 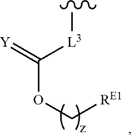 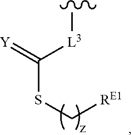 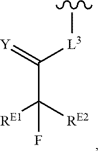 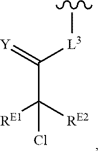 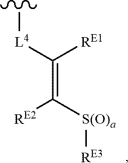 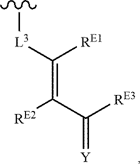 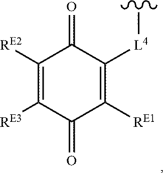 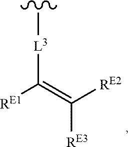 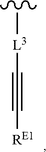 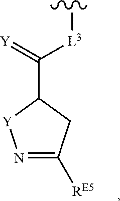 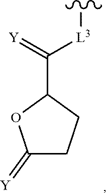 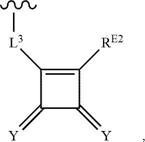 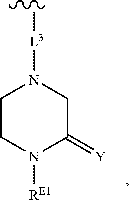 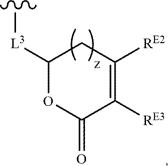 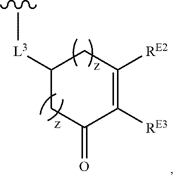 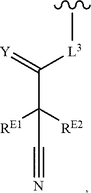 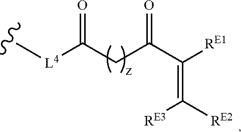 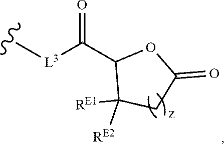 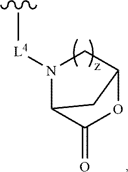 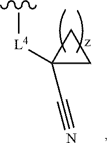 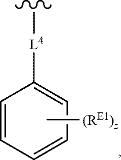 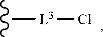 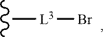 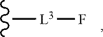 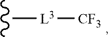 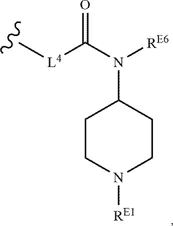 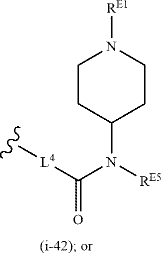 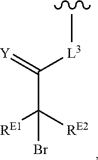
wherein:
L³ is a bond or an optionally substituted C₁₋₄ hydrocarbon chain, optionally wherein one or more carbon units of the hydrocarbon chain are independently replaced with —C═O—, —O—, —S—, —NR^{L3a}—, —NR^{L3a}C(═O)—, —C(═O)NR^{L3a}——SC(═O)—, —C(═O)S—, —OC(═O)—, —C(═O)O—, —NR^{L3a}C(═S)—, —C(═S)NR^{L3a}—, trans-CR^{L3b}=CR^{L3b}-, cis-CR^{L3b}=CR^{L3b}-, —C═C—, —S(═O)—, —S(═O)O—, —OS(═O)—, —S(═O)NR^{L3a}—, —NR ^{L3a}S(═O)—, —S(═O)₂—, —S(═O)20—, —OS(═O)₂—, —S(═O)₂NR^{L3a}—, or —NR^{L3a}S(—O)₂—, wherein R^{L3a} is hydrogen, substituted or unsubstituted C₁₋₆ alkyl, or a nitrogen protecting group, and wherein each occurrence of R^{L3b} is independently hydrogen, halogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or two R^{L3b} groups are joined to form an optionally substituted carbocyclic or optionally substituted heterocyclic ring;
L⁴ is a bond or an optionally substituted, branched or unbranched C₁₋₆ hydrocarbon chain;
each of R^{E1}, R^{E2}, and R^{E3} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —CH₂OR^{EE}, —CH₂N(R^{EE})₂, —CH₂SR^{EE}, —OR^{EE}, —N(R^{EE})₂, —Si(R^{EE})₃, or —SR^{EE}, wherein each instance of R^{EE} is independently hydrogen, optionally substituted alkyl, optionally substituted alkoxy, optionally substituted alkenyl, optionally substituted alkynyl, optionally substituted carbocyclyl, optionally substituted heterocyclyl, optionally substituted aryl, or optionally substituted heteroaryl, or two R^{EE} groups are joined to form an optionally substituted heterocyclic ring; or R^{E1} and R^{E3}, or R^{E2} and R^{E3}, or R^{E1} and R^{E2} are joined to form an optionally substituted carbocyclic or optionally substituted heterocyclic ring;
R^{E4} is a leaving group;
R^{E5} is halogen;
R^{E6} is hydrogen, substituted or unsubstituted C₁₋₆ alkyl, or a nitrogen protecting group;
each instance of Y is independently O, S, or NR^{E7}, wherein R^{E7} is hydrogen, substituted or unsubstituted C₁₋₆ alkyl, or a nitrogen protecting group;
a is 1 or 2; and
each instance of z is independently 0, 1, 2, 3, 4, 5, or 6, as valency permits;
provided that the compound is not of formula: 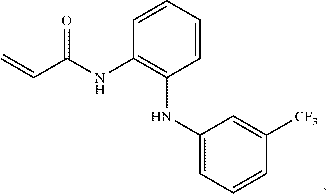 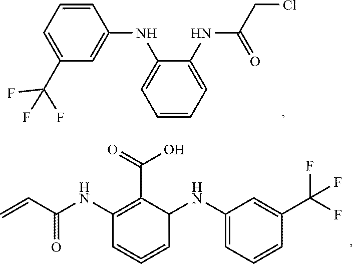 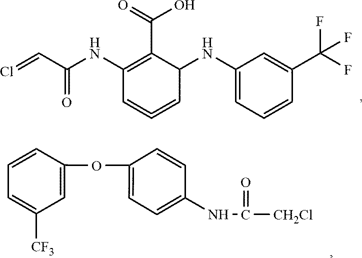 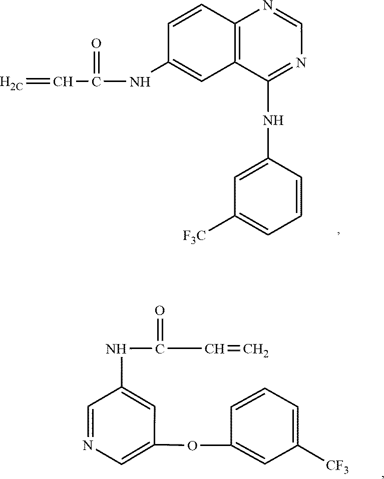 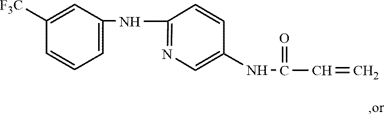 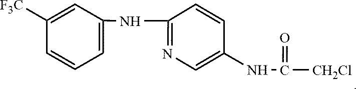
.

52. The compound of claim 51, wherein Z is —CH═.

53. The compound of claim 51 or 52, wherein W is —CH═.

54. The compound of claim 51 or 53, wherein Z is —N═.

55. The compound of claim 51, 52, or 54, wherein W is —N.

56. The compound of any one of claims 51-55, wherein Formula (II) is of the formula: 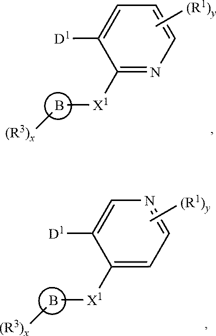 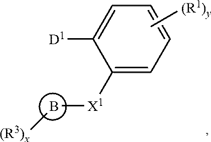
or a pharmaceutically acceptable salt, solvate, hydrate, polymorph, co-crystal, tautomer, stereoisomer, isotopically labeled derivative, or prodrug thereof.

57. The compound of any one of claims 51-55, wherein Formula (II) is of the formula: 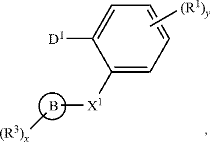
or a pharmaceutically acceptable salt, solvate, hydrate, polymorph, co-crystal, tautomer, stereoisomer, isotopically labeled derivative, or prodrug thereof.

58. The compound of any one of claims 51-57, wherein Ring B is cyclohexyl.

59. The compound of any one of claims 51-57, wherein Ring B is phenyl.

60. The compound of any one of claims 51-59, wherein x is 0.

61. The compound of any one of claims 51-59, wherein x is 1.

62. The compound of any one of claims 51-59, wherein x is 2.

63. The compound of any one of claims 51-62, wherein Ring B is of formula: 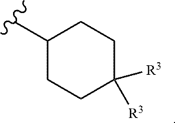
.

64. The compound of any one of claims 51-62, wherein Ring B is of formula: 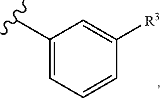 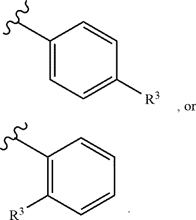
.

65. The compound of any one of claims 51-63, wherein Ring B is of formula: 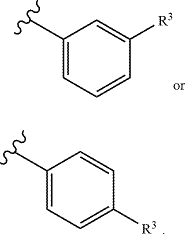
.

66. The compound of any one of claims 51-65, wherein at least one instance of R³ is halogen.

67. The compound of claim 51-66, wherein at least one instance of R³ is —F.

68. The compound of any one of claims 51-65, wherein at least one instance of R³ is optionally substituted alkyl.

69. The compound of any one of claims 51-65 or 68, wherein at least one instance of R³ is optionally substituted C₁₋₆ alkyl.

70. The compound of claim 51-65, 68, or 69, wherein at least one instance of R³ is —CF₃.

71. The compound of any one of claims 51-65, wherein at least one instance of R³ is optionally substituted carbocyclyl.

72. The compound of any one of claims 51-65 or 71, wherein at least one instance of R³ is optionally substituted C₃₋₁₄ carbocyclyl.

73. The compound of any one of claims 51-58, 60-63, 66, or 67, wherein the moiety 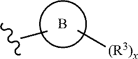
is of formula: 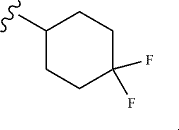
.

74. The compound of any one of claims 51-57, 59-62, 64, 65, or 68-73, wherein the moiety 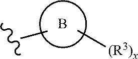
is of formula: 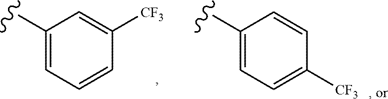 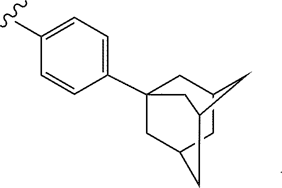
.

75. The compound of any one of claims 51-74, wherein y is 0.

76. The compound of any one of claims 51-74, wherein y is 1.

77. The compound of any one of claims 51-76, wherein at least one instance of R¹ is halogen.

78. The compound of any one of claims 51-77, wherein at least one instance of R¹ is —F.

79. The compound of any one of claims 51-78, wherein X¹ is —O—.

80. The compound of any one of claims 51-78, wherein X¹ is —O(CR^{d})_{1—6}—, and R^{d} is hydrogen, halogen, optionally substituted acyl, optionally substituted alkyl, optionally substituted alkenyl, optionally substituted alkynyl, optionally substituted carbocyclyl, —OR^{c1} —NO₂, —N(R^{c2})₂, —SR^{c1}, —CN, or —SCN.

81. The compound of any one of claims 51-78 or 80, wherein X¹ is of formula: 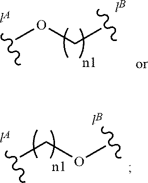
l^{A} indicates the point of attachment to the moiety of formula: 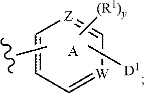
and l^{B} indicates the point of attachment to Ring B; and
n1 is 1, 2, 3, 4, 5, or 6.

82. The compound of any one of claims 51-78, 80, or 81, wherein X¹ is of formula: 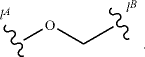
.

83. The compound of any one of claims 51-78, wherein X¹ is -N(R^{da})-, and R^{da} is hydrogen or optionally substituted C₁₋₆ alkyl.

84. The compound of any one of claims 51-78 or 83, wherein X¹ is —NH—.

85. The compound of any one of claims 51-84, wherein D¹ is of formula: 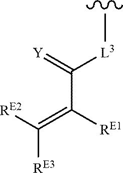
.

86. The compound of any one of claims 51-85, wherein D¹ is of formula: 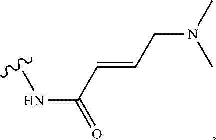 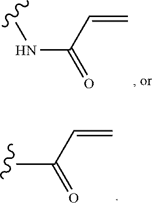
.

87. The compound of any one of claims 51-86, wherein D¹ is of formula: 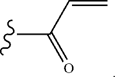
.

88. The compound of any one of claims 51-86, wherein D¹ is of formula: 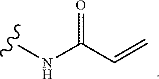
.

89. The compound of any one of claims 51-88, wherein the moiety of formula: 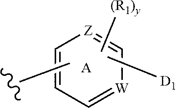
is of formula: 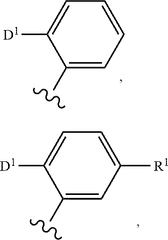 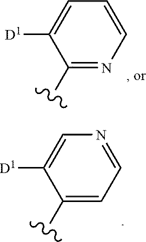
.

90. The compound of any one of claims 51-89, wherein the compound is of formula: 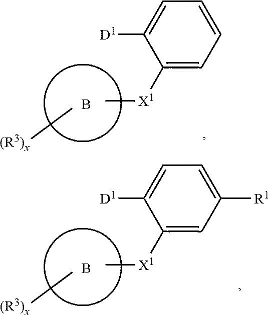 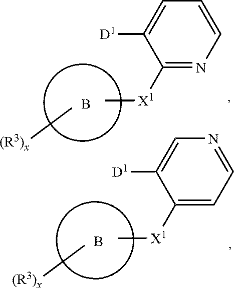
or a pharmaceutically acceptable salt, solvate, hydrate, polymorph, co-crystal, tautomer, stereoisomer, isotopically labeled derivative, or prodrug thereof.

91. The compound of any one of claims 51-90, wherein the compound is of formula: 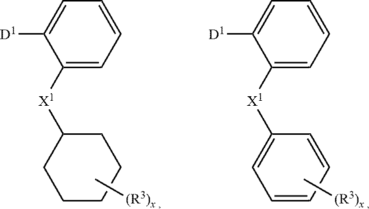 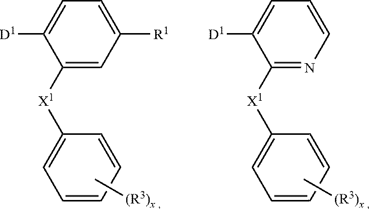 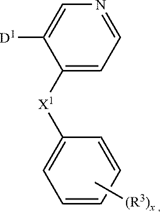
or a pharmaceutically acceptable salt, solvate, hydrate, polymorph, co-crystal, tautomer, stereoisomer, isotopically labeled derivative, or prodrug thereof.

92. The compound of any one of claims 51-91, wherein the compound is of formula: 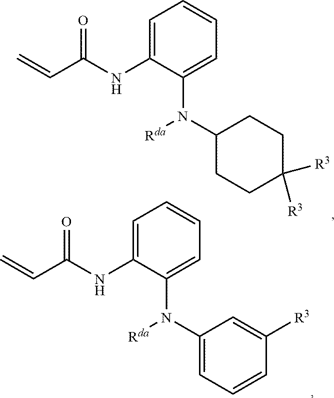 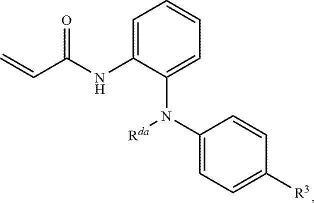 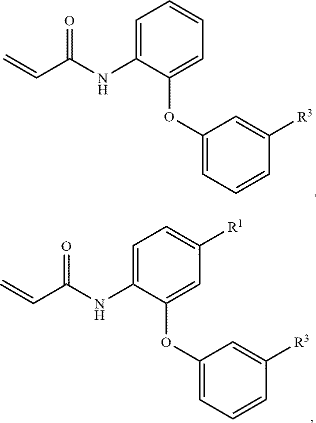 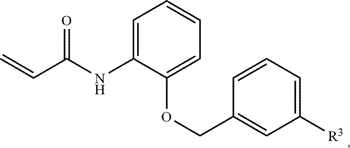 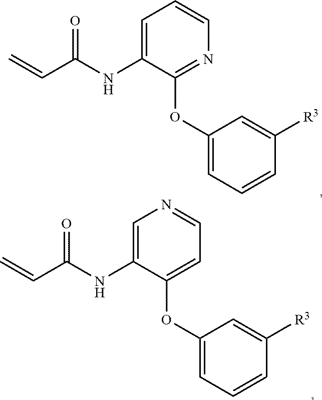
or a pharmaceutically acceptable salt, solvate, hydrate, polymorph, co-crystal, tautomer, stereoisomer, isotopically labeled derivative, or prodrug thereof.

93. The compound of any one of claims 51-92, wherein the compound is of formula: 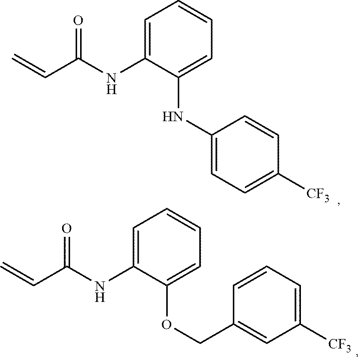 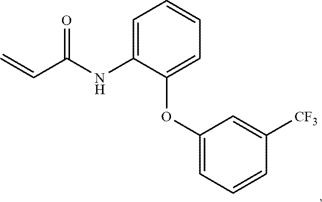 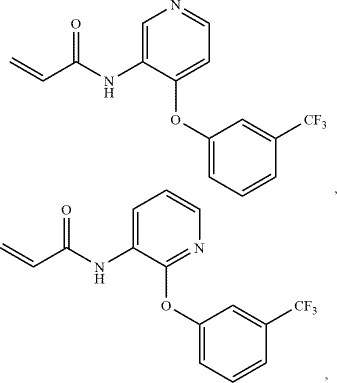 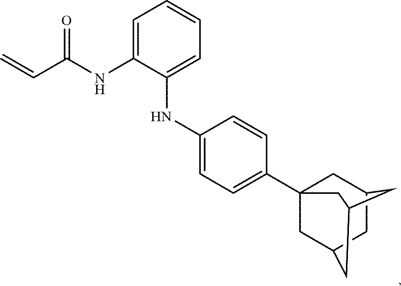 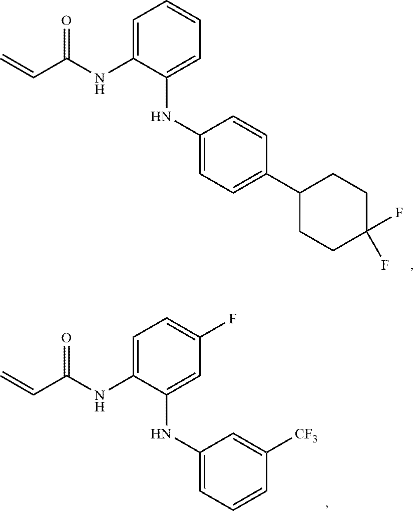
or a pharmaceutically acceptable salt, co-crystal, tautomer, stereoisomer, solvate, hydrate, polymorph, isotopically enriched derivative, or prodrug thereof.

94. A pharmaceutical composition comprising a compound of any one of claims 1-93, or a pharmaceutically acceptable salt, solvate, hydrate, polymorph, co-crystal, tautomer, stereoisomer, isotopically labeled derivative, or prodrug thereof, and optionally a pharmaceutically acceptable excipient.

95. The pharmaceutical composition of claim 94, wherein the pharmaceutical composition comprises a therapeutically effective amount of the compound for use in treating a proliferative disease in a subject in need thereof.

96. A method of treating a proliferative disease in a subject in need thereof, the method comprising administering to the subject a therapeutically effective amount of a compound of any one of claims 1-93, or a pharmaceutically acceptable salt, solvate, hydrate, polymorph, co-crystal, tautomer, stereoisomer, isotopically labeled derivative, or prodrug thereof, or a pharmaceutical composition of claim 94 or 95.

97. The method of claim 96, wherein the proliferative disease is cancer.

98. The method of claim 97, wherein the cancer is a sarcoma.

99. The method of claim 98, wherein the sarcoma is Kaposi’s sarcoma.

100. The method of claim 97, wherein the cancer is lung cancer.

101. The method of claim 100, wherein the lung cancer is non-small cell lung cancer.

102. The method of claim 100, wherein the lung cancer is mesothelioma.

103. The method of claim 97, wherein the cancer is a thyroid cancer.

104. The method of claim 97, wherein the cancer is breast cancer.

105. The method of claim 97, wherein the cancer is liver cancer.

106. The method of claim 97, wherein the cancer is prostate cancer.

107. The method of claim 97, wherein the cancer is pancreatic cancer.

108. The method of claim 97, wherein the cancer is colorectal cancer.

109. The method of claim 97, wherein the cancer is ovarian cancer.

110. The method of claim 97, wherein the cancer is skin cancer.

111. The method of claim 97, wherein the cancer is esophageal cancer.

112. The method of claim 97, wherein the cancer is a carcinoma.

113. The method of claim 97, wherein the cancer is fallopian tube carcinoma.

114. The method of claim 97, wherein the cancer is resistant to inhibitors of EGFR or MEK.

115. The method of claim 96, wherein the proliferative disease is an inflammatory disease.

116. The method of claim 115, wherein the inflammatory disease is fibrosis.

117. The method of claim 96, wherein the proliferative disease is an autoimmune disease.

118. The method of claim 117, wherein the autoimmune disease is sclerosis.

119. A method of inhibiting a transcription factor in a subject in need thereof, the method comprising:
administering to the subject a therapeutically effective amount of a compound of any one of claims 1-93, or a pharmaceutically acceptable salt, solvate, hydrate, polymorph, co-crystal, tautomer, stereoisomer, isotopically labeled derivative, or prodrug thereof, or a pharmaceutical composition of claim 94 or 95.

120. A method of inhibiting a transcription factor in a biological sample, the method comprising:
contacting the biological sample with an effective amount of a compound of any one of claims 1-93, or a pharmaceutically acceptable salt, solvate, hydrate, polymorph, co-crystal, tautomer, stereoisomer, isotopically labeled derivative, or prodrug thereof, or a pharmaceutical composition of claim 94 or 95.

121. A method of inhibiting the transcription of a gene in a subject in need thereof, the method comprising:
administering to the subject a therapeutically effective amount of a compound of any one of claims 1-93, or a pharmaceutically acceptable salt, solvate, hydrate, polymorph, co-crystal, tautomer, stereoisomer, isotopically labeled derivative, or prodrug thereof, or a pharmaceutical composition of claim 94 or 95.

122. A method of inhibiting the transcription of a gene in a biological sample, the method comprising:
contacting the biological sample with an effective amount of a compound of any one of claims 1-93, or a pharmaceutically acceptable salt, solvate, hydrate, polymorph, co-crystal, tautomer, stereoisomer, isotopically labeled derivative, or prodrug thereof, or a pharmaceutical composition of claim 94 or 95.

123. The method of claim 121 or 122, wherein the gene is controlled or regulated by a transcription factor.

124. The method of any one of claims 119, 120, or 123, wherein the transcription factor is TEAD1, TEAD2, TEAD3, or TEAD4.

125. The method of claim 124, wherein the transcription factor is TEAD1.

126. The method of claim 124, wherein the transcription factor is TEAD2.

127. The method of claim 126, wherein the compound is capable of covalently binding TEAD2.

128. The method of claim 126 or 127, wherein the compound is capable of covalently binding a cysteine residue of TEAD2.

129. The method of claim 124, wherein the transcription factor is TEAD4.

130. The method of any one of claims 119-122, wherein the compound is capable of binding the YAP/TAZ domain of a TEAD family transcription factor.

131. The method of claim 120 or 122, wherein the biological sample is a tissue or cell.

132. Use of a compound of any one of claims 1-93, or a pharmaceutically acceptable salt, solvate, hydrate, polymorph, co-crystal, tautomer, stereoisomer, isotopically labeled derivative, or prodrug thereof, or a pharmaceutical composition of claim 94 or 95, for treating a disease in a subject in need thereof.

133. A compound of any one of claims 1-93, or a pharmaceutically acceptable salt, solvate, hydrate, polymorph, co-crystal, tautomer, stereoisomer, isotopically la—led derivative, or prodrug thereof, or a pharmaceutical composition of claim 94 or 95, for use in treating a disease in a subject in need thereof.

134. A kit comprising: a compound of any one of claims 1-93, or a pharmaceutically acceptable salt, solvate, hydrate, polymorph, co-crystal, tautomer, stereoisomer, isotopically labeled derivative, or prodrug thereof, or a pharmaceutical composition of claim 94 or 95; and
instructions for administering to a subject or contacting a biological sample with the compound, or the pharmaceutically acceptable salt, solvate, hydrate, polymorph, co-crystal, tautomer, stereoisomer, isotopically labeled derivative, or prodrug thereof, or the pharmaceutical composition.
